(19) United States
(12) Patent Application Publication
(10) Pub. No.: US 2023/0322802 A1
(43) Pub. Date: Oct. 12, 2023

(54) LIGHT EMITTING DEVICE AND FUSED POLYCYCLIC COMPOUND FOR THE LIGHT EMITTING DEVICE

(71) Applicant: Samsung Display Co., Ltd., Yongin-si (KR)

(72) Inventors: Kyoung SUNWOO, Hwaseong-si (KR); TAEIL KIM, Hwaseong-si (KR); JUNHA PARK, Gwacheon-si (KR); MinJae SUNG, Hwaseong-si (KR); MUN-KI SIM, Seoul (KR); MINJUNG JUNG, Hongcheon-gun (KR)

(21) Appl. No.: 18/114,158

(22) Filed: Feb. 24, 2023

(30) Foreign Application Priority Data
Apr. 8, 2022 (KR) 10-2022-0044199

(51) Int. Cl. C07D 495/22; H10K 50/11; H10K 85/60

(52) U.S. Cl. CPC C07D 495/22; H10K 50/11; H10K 85/657; H10K 85/6572; C07B 2200/05

(57) ABSTRACT

A light emitting device includes a first electrode, a second electrode facing the first electrode, and an emission layer disposed between the first electrode and the second electrode, wherein the emission layer includes a first compound represented by Formula 1 below:

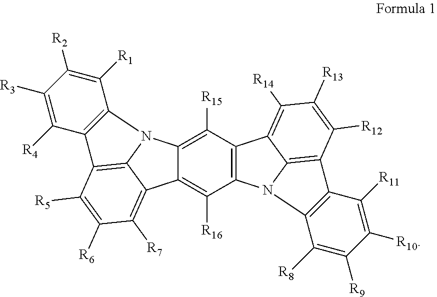

### CROSS-REFERENCE TO RELATED APPLICATION

This application claims priority to and the benefit of Korean Patent Application No. 10-2022-0044199, filed on Apr. 8, 2022, in the Korean Intellectual Property Office, the entire content of which is hereby incorporated by reference.

### BACKGROUND

### 1. Field

The present disclosure herein relates to a light emitting device and a fused polycyclic compound for the light emitting device.

### 2. Description of Related Art

Recently, the development of an organic electroluminescence display apparatus as an image display apparatus is being actively conducted. Unlike liquid crystal display apparatuses and/or the like, the organic electroluminescence display apparatus is a so-called self-luminescent display apparatus in which holes and electrons injected from a first electrode and a second electrode recombine in an emission layer, and thus a luminescent material including an organic compound in the emission layer emits light to implement (e.g., realize) display.

In the application of an organic electroluminescence device to a display apparatus, there is a demand for an organic electroluminescence device having a low driving voltage, a high luminous efficiency, and a long service life, and the development on materials, for an organic electroluminescence device, capable of stably attaining such characteristics is being continuously pursued.

In recent years, particularly in order to implement a highly efficient organic electroluminescence device, technologies pertaining to phosphorescence emission utilizing triplet state energy or delayed fluorescence utilizing triplet-triplet annihilation (TTA) in which singlet excitons are generated by collision of triplet excitons are being developed, and thermally activated delayed fluorescence (TADF) materials utilizing delayed fluorescence phenomenon are being developed.

### SUMMARY

Aspects according to embodiments of the present disclosure are directed toward a light emitting device in which luminous efficiency and a device service life are improved.

Aspects according to embodiments of the present disclosure are directed toward a fused polycyclic compound capable of improving luminous efficiency and a device service life of a light emitting device.

Additional aspects will be set forth in part in the description which follows and, in part, will be apparent from the description, or may be learned by practice of the presented embodiments of the disclosure.

According to an embodiment of the present disclosure, a light emitting device includes a first electrode, a second electrode facing the first electrode, and an emission layer between the first electrode and the second electrode, wherein the emission layer includes a first compound represented by Formula 1:

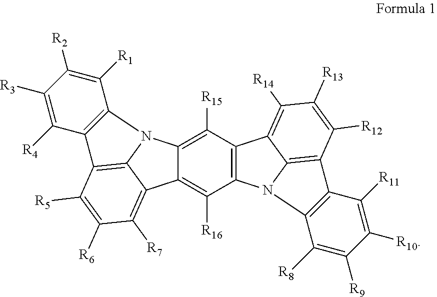

In Formula 1, R₁ to R₁₆ may each independently be a hydrogen atom, a deuterium atom, a halogen atom, a cyano group, a substituted or unsubstituted silyl group, a substituted or unsubstituted amine group, a substituted or unsubstituted oxy group, a substituted or unsubstituted thio group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and/or bonded to an adjacent group to form a ring, and at least one pair from among adjacent groups of R₁ to R₁₄ is a position to which a substituent represented by Formula 2 is fused (e.g., the at least one pair of adjacent groups selected from among R₁ to R₁₄ may be bonded to each other to form the substituent represented by Formula 2):

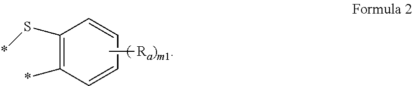

In Formula 2, —* is a position fused to any one adjacent pair selected from among R₁ to R₁₄ in Formula 1 (e.g., is a bonding site to a core of Formula 1), Rₐ is a hydrogen atom, a deuterium atom, a halogen atom, a cyano group, a substituted or unsubstituted silyl group, a substituted or unsubstituted amine group, a substituted or unsubstituted oxy group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and/or bonded to an adjacent group to form a ring, and m1 is an integer of 0 to 4.

In an embodiment, the first compound represented by Formula 1 may be represented by Formula 3-1 or Formula 3-2:

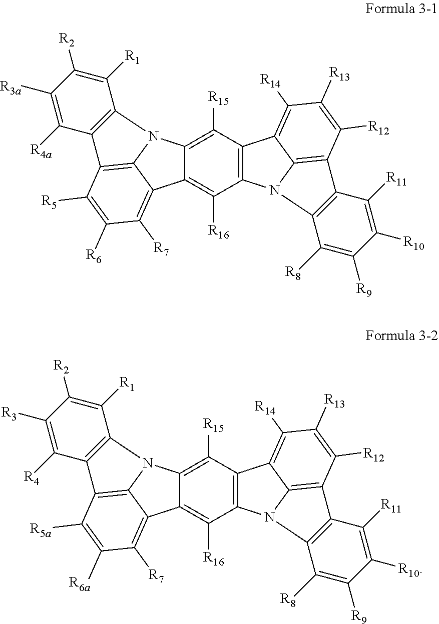

In Formula 3-1, each of R₃ₐ and R₄ₐ may be a position to which a substituent represented by Formula 2 is fused (e.g., R₃ₐ and R₄ₐ may be bonded to each other to form the substituent represented by Formula 2), and in Formula 3-2, each of R₅ₐ and R₆ₐ may be a position to which a substituent represented by Formula 2 is fused (e.g., R₅ₐ and R₆ₐ may be bonded to each other to form the substituent represented by Formula 2).

In Formula 3-1 and Formula 3-2, the same as defined in Formula 1 may be applied to R₁ to R₁₆.

In an embodiment, the first compound represented by Formula 1 may be represented by Formula 4-1 or Formula 4-2:

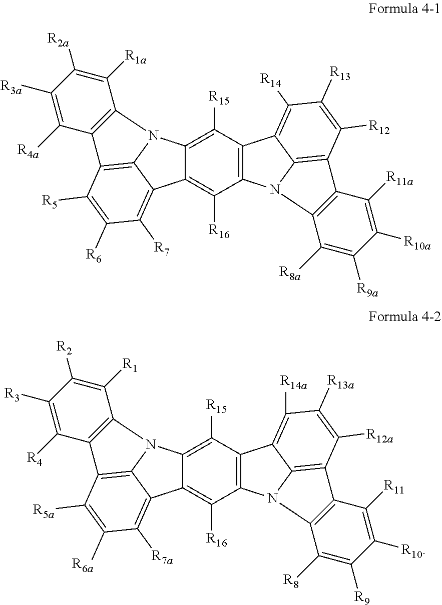

In Formula 4-1 and Formula 4-2, R₁ₐ to R₁₄ₐ may each independently be a hydrogen atom, a deuterium atom, a halogen atom, a cyano group, a substituted or unsubstituted silyl group, a substituted or unsubstituted amine group, a substituted or unsubstituted oxy group, a substituted or unsubstituted thio group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and/or bonded to an adjacent group to form a ring, and in Formula 4-1, one adjacent pair selected from among R₁ₐ to R₄ₐ and one adjacent pair selected from among R₈ₐ to R₁₁ₐ may each be positions to which a substituent represented by Formula 2 is fused (e.g., one pair of adjacent groups selected from among R₁ₐ to R₄ₐ may be bonded to each other to form the substituent represented by Formula 2, and one pair of adjacent groups selected from among R₈ₐ to R₁₁ₐ may be bonded to each other to form the substituent represented by Formula 2), and in Formula 4-2, one adjacent pair selected from among R₅ₐ to R₇ₐ and one adjacent pair selected from among R₁₂ₐ to R₁₄ₐ may each be positions to which a substituent represented by Formula 2 is fused (e.g., one pair of adjacent groups selected from among R₅ₐ to R₇ₐ may be bonded to each other to form the substituent represented by Formula 2, and one pair of adjacent groups selected from among R₁₂ₐ to R₁₄ₐ may be bonded to each other to form the substituent represented by Formula 2).

In Formula 4-1 and Formula 4-2, the same as defined in Formula 1 may be applied to R₁ to R₁₆.

In an embodiment, the first compound represented by Formula 1 may be represented by Formula 5-1 or Formula 5-2:

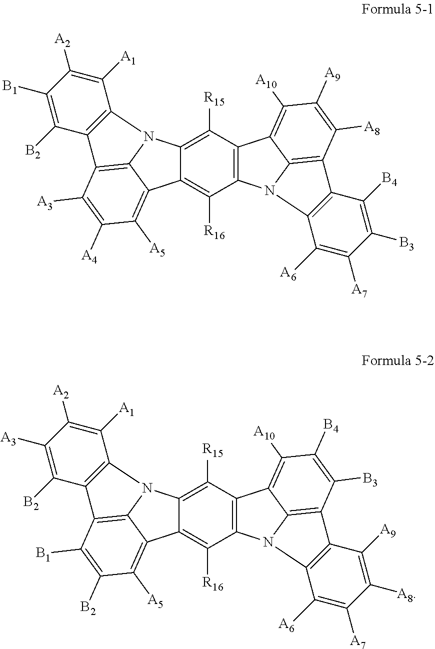

In Formula 5-1 and Formula 5-2, A₁ to A₁₀ may each independently be a hydrogen atom, a deuterium atom, a halogen atom, a cyano group, a substituted or unsubstituted silyl group, a substituted or unsubstituted amine group, a substituted or unsubstituted oxy group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and/or bonded to an adjacent group to form a ring, and B₁ and B₂, and B₃ and B₄ may each be positions to which a substituent represented by Formula 2 is fused (e.g., B₁ and B₂ may be bonded to each other to form the substituent represented by Formula 2, and B₃ and B₄ may be bonded to each other to form the substituent represented by Formula 2).

In Formula 5-1 and Formula 5-2, the same as defined in Formula 1 may be applied to R₁₅ and R₁₆.

In an embodiment, the first compound represented by Formula 1 may be represented by any one from among Formula 6-1 to Formula 6-4:

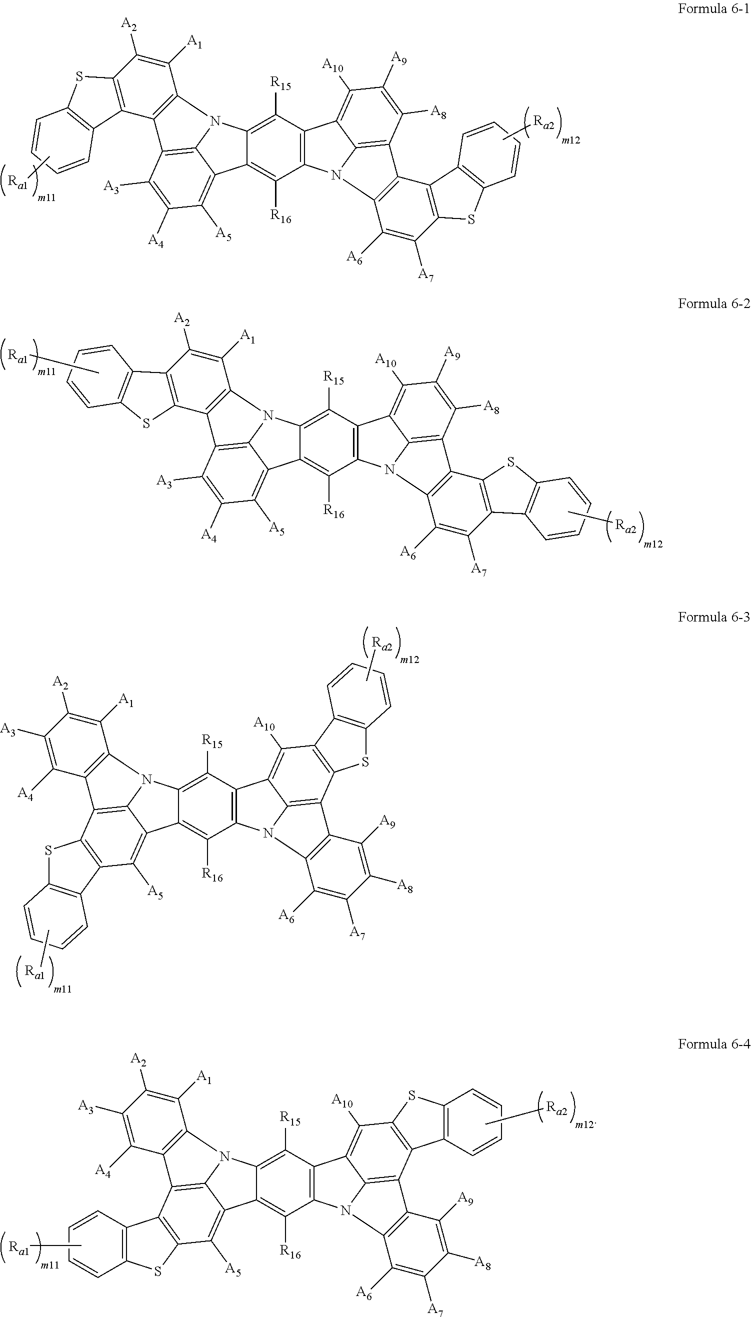

In Formula 6-1 to Formula 6-4, Rₐ₁ and Rₐ₂ may each independently be a hydrogen atom, a deuterium atom, a halogen atom, a cyano group, a substituted or unsubstituted silyl group, a substituted or unsubstituted amine group, a substituted or unsubstituted oxy group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and/or bonded to an adjacent group to form a ring, and m11 and m12 may each independently be an integer of 0 to 4.

In Formula 6-1 to Formula 6-4, the same as defined in Formula 1, Formula 5-1, and Formula 5-2 may be applied to R₁₅, R₁₆, and A₁ to A₁₀.

In an embodiment, the first compound represented by Formula 1 may be represented by any one from among Formula 7-1 to Formula 7-4:

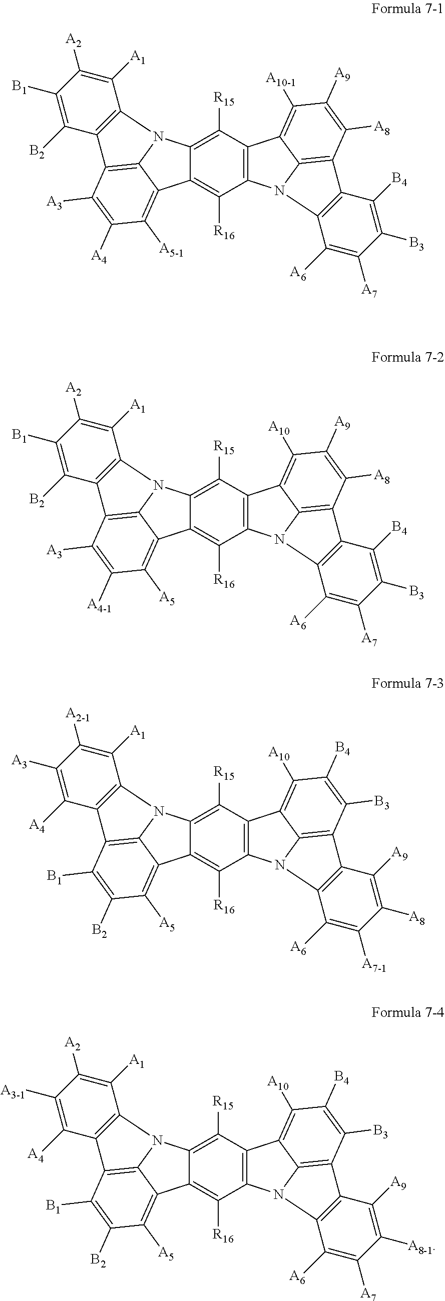

In Formula 7-1 to Formula 7-4, A2-1 to A₅₋₁ and A₇₋₁ to A₁₀₋₁ may each independently be a substituted or unsubstituted aryl group having 6 to 30 ring-forming carbon atoms, or a substituted or unsubstituted heteroaryl group having 2 to 30 ring-forming carbon atoms.

In Formula 7-1 to Formula 7-4, the same as defined in Formula 1, Formula 5-1, and Formula 5-2 may be applied to R₁₅, R₁₆, A₁ to A₁₀, and B₁ to B₄.

In an embodiment, A₂₋₁ to A₅₋₁ and A₇₋₁ to A₁₀₋₁ may each independently be represented by any one from among Formula 8-1 to Formula 8-4:

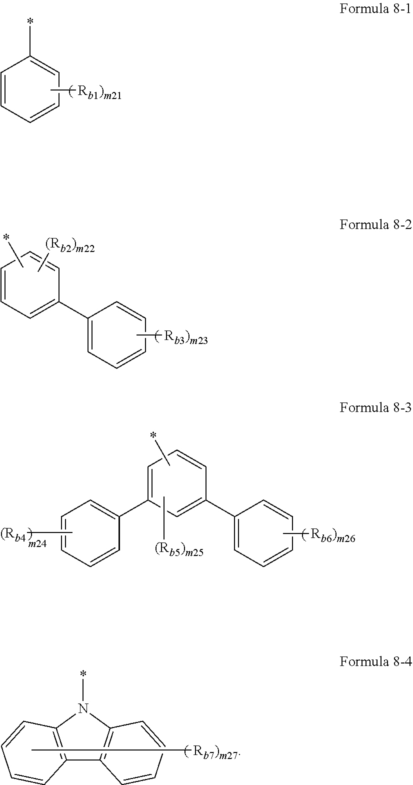

In Formula 8-1 to Formula 8-4, R_{b1} to R_{b7} may each independently be a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, m21, m23, m24, and m26 may each independently be an integer of 0 to 5, m22 may be an integer of 0 to 4, and m25 may be an integer of 0 to 3, and m27 may be an integer of 0 to 8.

In an embodiment, the first compound represented by Formula 1 may be represented by Formula 9:

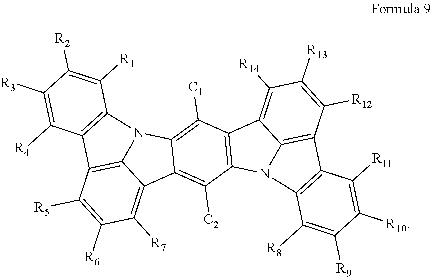

In Formula 9, C₁ and C₂ may each independently be a hydrogen atom or a deuterium atom.

In Formula 9, the same as defined in Formula 1 may be applied to R₁ to R₁₄.

In an embodiment, the emission layer may further include a second compound represented by Formula H-1:

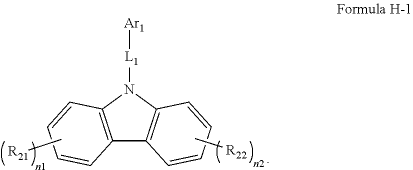

In Formula H-1, Li may be a direct linkage, a substituted or unsubstituted arylene group having 6 to 30 ring-forming carbon atoms, or a substituted or unsubstituted heteroarylene group having 2 to 30 ring-forming carbon atoms, An may be a substituted or unsubstituted aryl group having 6 to 30 ring-forming carbon atoms, or a substituted or unsubstituted heteroaryl group having 2 to 30 ring-forming carbon atoms, R₂₁ and R₂₂ may each independently be a hydrogen atom, a deuterium atom, a halogen atom, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, and/or bonded to an adjacent group to form a ring, and n1 and n2 may each independently be an integer of 0 to 4.

In an embodiment, the emission layer may further include a third compound represented by Formula H-2:

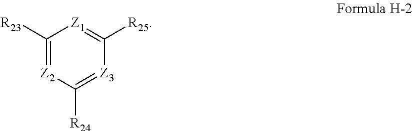

In Formula H-2, Z₁ to Z₃ may each independently be N or CR₂₆, at least one from among Z₁ to Z₃ may be N, and R₂₃ to R₂₆ may each independently be a hydrogen atom, a deuterium atom, a halogen atom, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, and/or bonded to an adjacent group to form a ring.

In an embodiment, the emission layer may further include a fourth compound represented by Formula D-1:

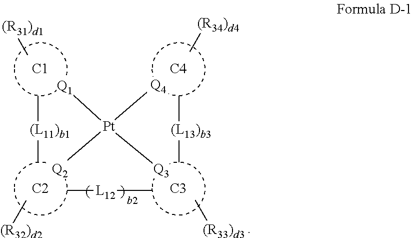

In Formula D-1, Q₁ to Q₄ may each independently be C or N, C1 to C4 may each independently be a substituted or unsubstituted hydrocarbon ring having 5 to 30 ring-forming carbon atoms, or a substituted or unsubstituted heterocycle having 2 to 30 ring-forming carbon atoms, L₁₁ to L₁₃ may each independently be a direct linkage,

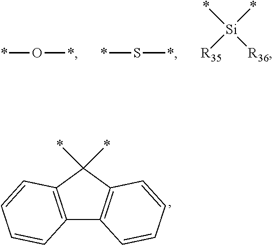

a substituted or unsubstituted divalent alkyl group having 1 to 20 carbon atoms, a substituted or unsubstituted arylene group having 6 to 30 ring-forming carbon atoms, or a substituted or unsubstituted heteroarylene group having 2 to 30 ring-forming carbon atoms, b1 to b3 may each independently be 0 or 1, R₃₁ to R₃₆ may each independently be a hydrogen atom, a deuterium atom, a halogen atom, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, and/or bonded to an adjacent group to form a ring, and d1 to d4 may each independently be an integer of 0 to 4.

### BRIEF DESCRIPTION OF THE DRAWINGS

The accompanying drawings are included to provide a further understanding of the present disclosure, and are incorporated in and constitute a part of this specification. The drawings illustrate example embodiments of the present disclosure and, together with the description, serve to explain principles of the present disclosure.

### IN THE DRAWINGS

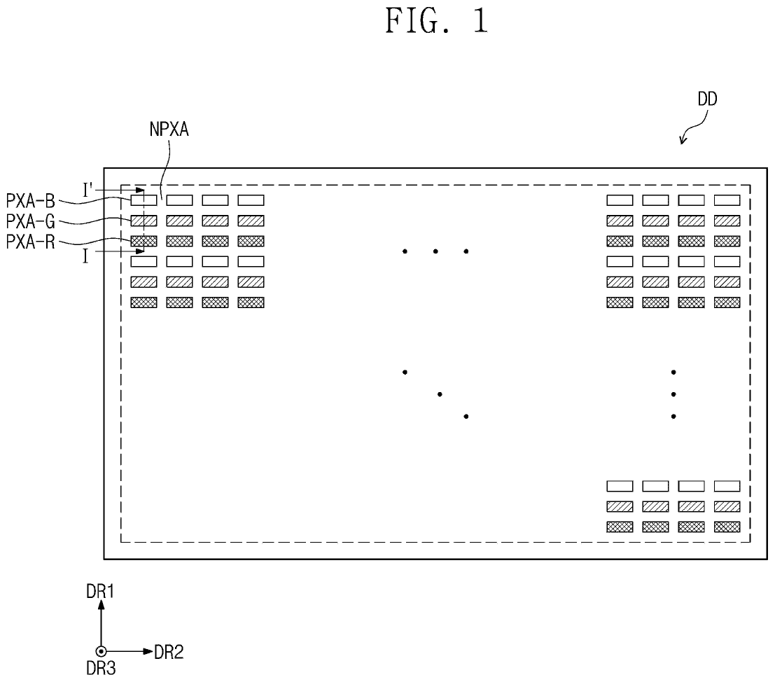

FIG. 1 is a plan view of a display apparatus according to an embodiment of the present disclosure;

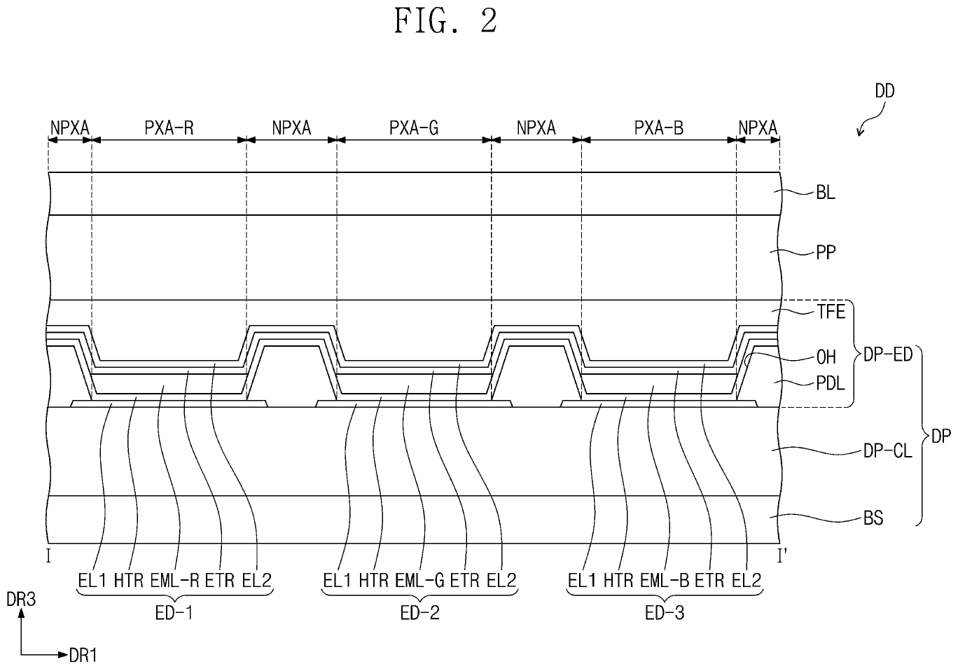

FIG. 2 is a cross-sectional view of a display apparatus according to an embodiment of the present disclosure;

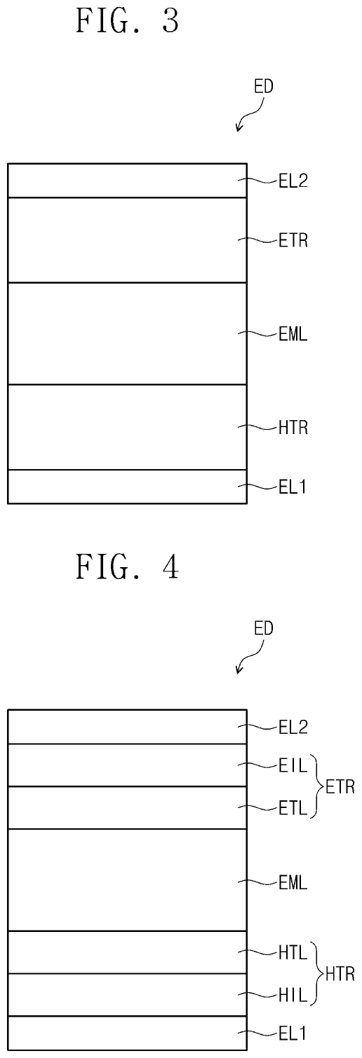

FIG. 3 is a cross-sectional view schematically illustrating a light emitting device according to an embodiment of the present disclosure;

FIG. 4 is a cross-sectional view schematically illustrating a light emitting device according to an embodiment of the present disclosure;

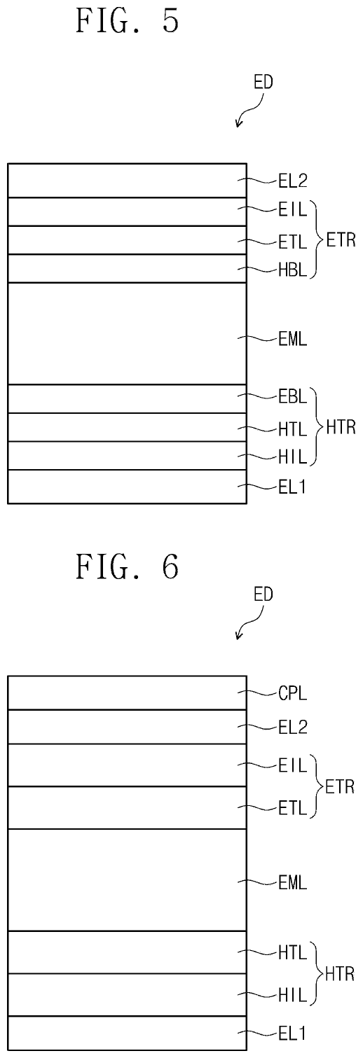

FIG. 5 is a cross-sectional view schematically illustrating a light emitting device according to an embodiment of the present disclosure;

FIG. 6 is a cross-sectional view schematically illustrating a light emitting device according to an embodiment of the present disclosure;

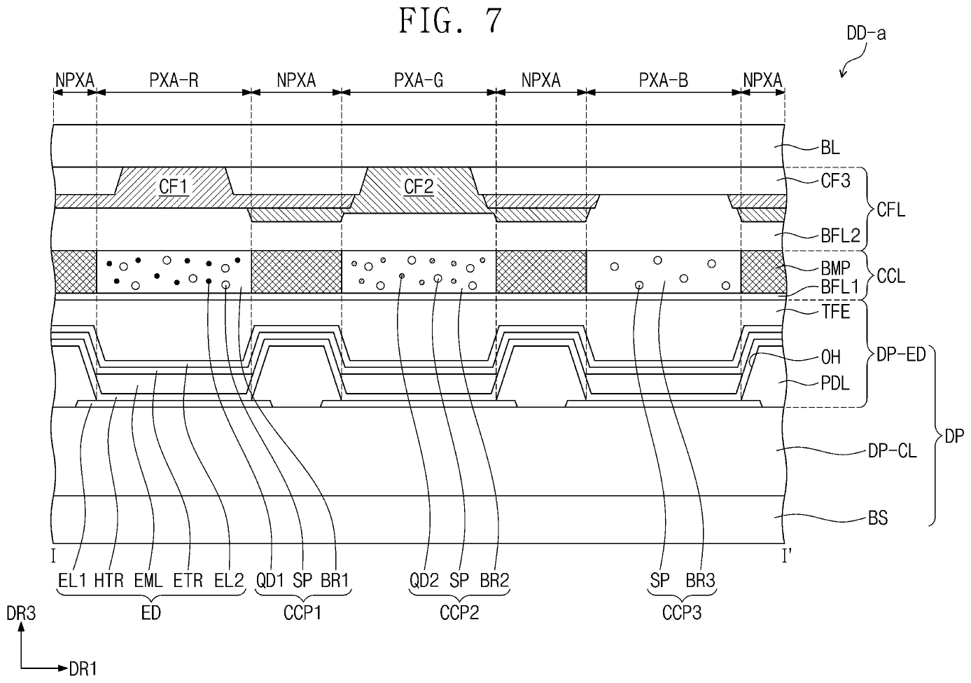

FIG. 7 is a cross-sectional view of a display apparatus according to an embodiment of the present disclosure;

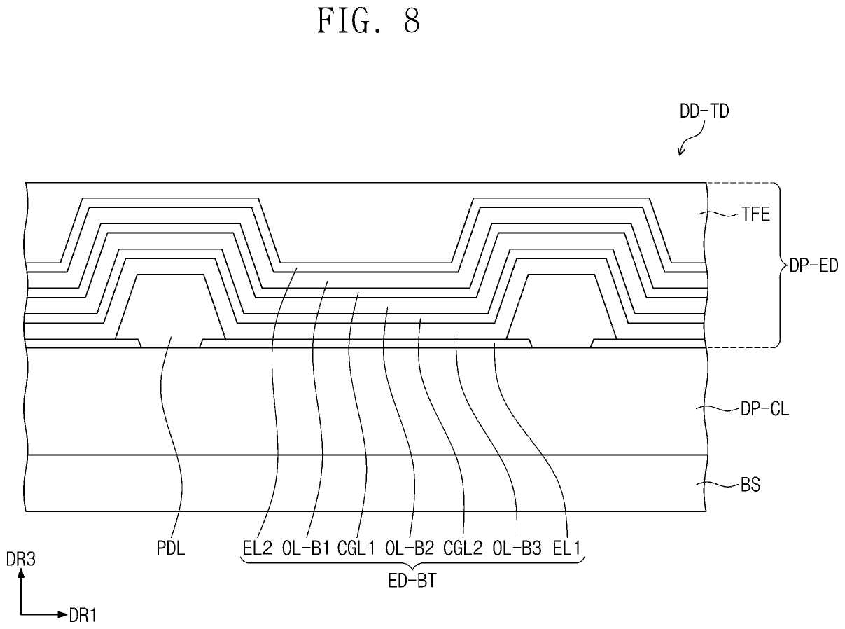

FIG. 8 is a cross-sectional view of a display apparatus according to an embodiment of the present disclosure;

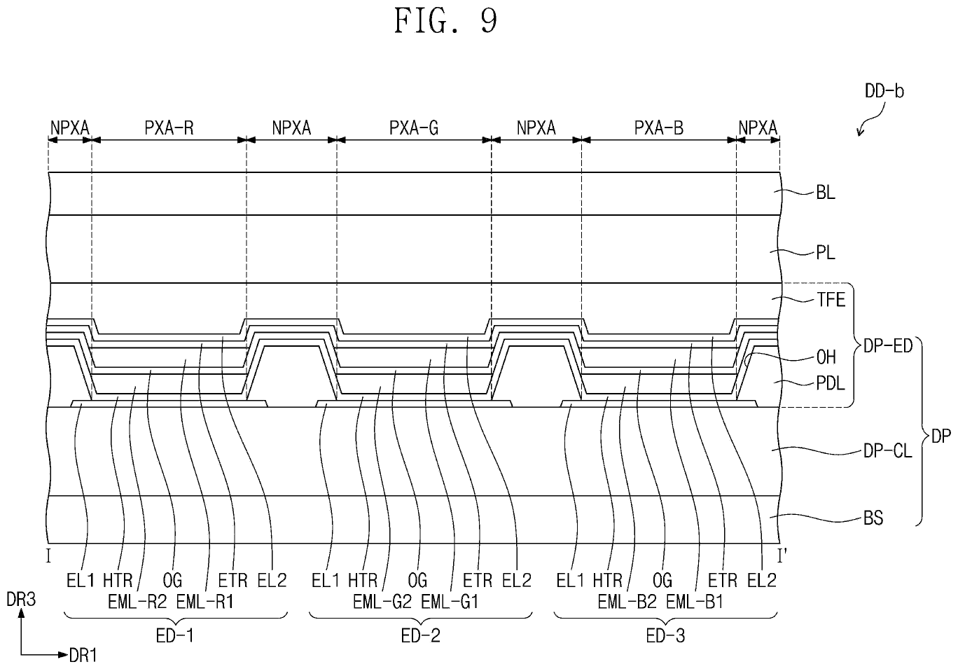

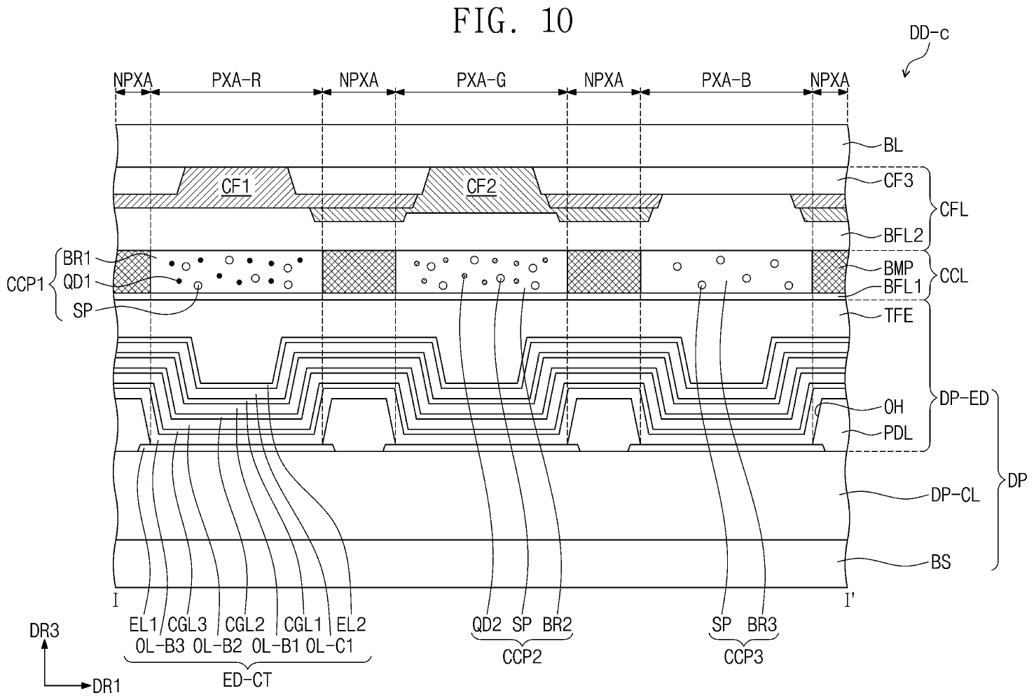

FIG. 9 is a cross-sectional view illustrating a display apparatus according to an embodiment of the present disclosure; and

FIG. 10 is a cross-sectional view illustrating a display apparatus according to an embodiment of the present disclosure.

### DETAILED DESCRIPTION

The present disclosure may be modified in many alternate forms, and thus specific embodiments will be explained in the drawings and described in more detail. It should be understood, however, that it is not intended to limit the present disclosure to the particular forms disclosed, but rather, is intended to cover all modifications, equivalents, and alternatives falling within the spirit and scope of the present disclosure.

When explaining each of the drawings, like reference numbers are used for referring to like elements. In the accompanying drawings, the dimensions of each structure may be exaggerated for clarity of the present disclosure. It will be understood that, although the terms “first,” “second,” etc. may be used herein to describe one or more suitable elements, these elements should not be limited by these terms. These terms are only utilized to distinguish one element from another. For example, a first element could be termed a second element, and, similarly, a second element could be termed a first element, without departing from the scope of example embodiments of the present disclosure. The terms of a singular form may include plural forms unless the context clearly indicates otherwise.

In the present application, it will be understood that the terms “include,” “have” and/or the like specify the presence of features, numbers, steps, operations, components, parts, or combinations thereof disclosed in the specification, but do not exclude the possibility of presence or addition of one or more other features, numbers, steps, operations, components, parts, or combinations thereof.

In the present application, when a layer, a film, a region, or a plate is referred to as being “above” or “on an upper portion of” another layer, film, region, or plate, it can be not only “directly on” the other layer, film, region, or plate, but intervening layers, films, regions, or plates may also be present. On the contrary, when a part such as a layer, a film, a region, or a plate is referred to as being “under” or “on a lower portion of” another part, it can be not only “directly under” the other part, but an intervening part may also be present. In addition, it will be understood that when a part is referred to as being “on” another part, it can be disposed above the other part, or disposed under the other part as well.

In the specification, the term “substituted or unsubstituted” may refer to a functional group that is substituted or unsubstituted with at least one substituent selected from the group consisting of a deuterium atom, a halogen atom, a cyano group, a nitro group, an amino group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, an alkoxy group, a hydrocarbon ring group, an aryl group, and a heterocyclic group. In addition, each of the substituents exemplified above may be substituted or unsubstituted. For example, a biphenyl group may be interpreted as an aryl group or a phenyl group substituted with a phenyl group.

In the specification, the phrase “bonded to an adjacent group to form a ring” may indicate that a group is bonded to an adjacent group to form a substituted or unsubstituted hydrocarbon ring, or a substituted or unsubstituted heterocycle. The hydrocarbon ring includes an aliphatic hydrocarbon ring and an aromatic hydrocarbon ring. The heterocycle includes an aliphatic heterocycle and an aromatic heterocycle.

The hydrocarbon ring and the heterocycle may be monocyclic or polycyclic. In addition, the rings formed by being bonded to each other may be connected to another ring to form a spiro structure.

In the specification, the term “adjacent group” may refer to a substituent substituted for an atom which is directly linked to an atom substituted with a corresponding substituent, another substituent substituted for an atom which is substituted with a corresponding substituent, or a substituent sterically positioned at the nearest position to a corresponding substituent. For example, two methyl groups in 1,2-dimethylbenzene may be interpreted as “adjacent groups” to each other and two ethyl groups in 1,1-diethylcyclopentane may be interpreted as “adjacent groups” to each other. In addition, two methyl groups in 4,5-dimethylphenanthrene may be interpreted as “adjacent groups” to each other.

In the specification, examples of the halogen atom may include a fluorine atom, a chlorine atom, a bromine atom, or an iodine atom.

In the specification, the alkyl group may be a linear, branched or cyclic alkyl group. The number of carbon atoms in the alkyl group may be 1 to 50, 1 to 30, 1 to 20, 1 to 10, or 1 to 6. Examples of the alkyl group may include a methyl group, an ethyl group, an n-propyl group, an isopropyl group, an n-butyl group, an s-butyl group, a t-butyl group, an i-butyl group, a 2-ethylbutyl group, a 3,3-dimethylbutyl group, an n-pentyl group, an i-pentyl group, a neopentyl group, a t-pentyl group, a cyclopentyl group, a 1-methylpentyl group, a 3-methylpentyl group, a 2-ethylpentyl group, a 4-methyl-2-pentyl group, an n-hexyl group, a 1-methylhexyl group, a 2-ethylhexyl group, a 2-butylhexyl group, a cyclohexyl group, a 4-methylcyclohexyl group, a 4-t-butylcyclohexyl group, an n-heptyl group, a 1-methylheptyl group, a 2,2-dimethylheptyl group, a 2-ethylheptyl group, a 2-butylheptyl group, an n-octyl group, a t-octyl group, a 2-ethyloctyl group, a 2-butyloctyl group, a 2-hexyloctyl group, a 3,7-dimethyloctyl group, a cyclooctyl group, an n-nonyl group, an n-decyl group, an adamantyl group, a 2-ethyldecyl group, a 2-butyldecyl group, a 2-hexyldecyl group, a 2-octyldecyl group, an n-undecyl group, an n-dodecyl group, a 2-ethyldodecyl group, a 2-butyldodecyl group, a 2-hexyldocecyl group, a 2-octyldodecyl group, an n-tridecyl group, an n-tetradecyl group, an n-pentadecyl group, an n-hexadecyl group, a 2-ethylhexadecyl group, a 2-butylhexadecyl group, a 2-hexylhexadecyl group, a 2-octylhexadecyl group, an n-heptadecyl group, an n-octadecyl group, an n-nonadecyl group, an n-eicosyl group, a 2-ethyleicosyl group, a 2-butyleicosyl group, a 2-hexyleicosyl group, a 2-octyleicosyl group, an n-heneicosyl group, an n-docosyl group, an n-tricosyl group, an n-tetracosyl group, an n-pentacosyl group, an n-hexacosyl group, an n-heptacosyl group, an n-octacosyl group, an n-nonacosyl group, an n-triacontyl group, etc., but the embodiment of the present disclosure is not limited thereto.

In the specification, the alkenyl group refers to a hydrocarbon group including at least one carbon-carbon double bond in the middle and/or at the terminal end of an alkyl group having 2 or more carbon atoms. The alkenyl group may be a linear chain or a branched chain. The number of carbon atoms in the alkenyl group is not specifically limited, but may be 2 to 30, 2 to 20, or 2 to 10. Examples of the alkenyl group may include a vinyl group, a 1-butenyl group, a 1-pentenyl group, a 1,3-butadienyl aryl group, a styrenyl group, a styryl vinyl group, etc., but the embodiment of the present disclosure is not limited thereto.

In the specification, the hydrocarbon ring group refers to any functional group or substituent derived from an aliphatic hydrocarbon ring. The hydrocarbon ring group may be a saturated hydrocarbon ring group having 5 to 20 ring-forming carbon atoms.

In the specification, an aryl group refers to any functional group or substituent derived from an aromatic hydrocarbon ring. The aryl group may be a monocyclic aryl group or a polycyclic aryl group. The number of ring-forming carbon atoms in the aryl group may be 6 to 30, 6 to 20, or 6 to 15. Examples of the aryl group may include a phenyl group, a naphthyl group, a fluorenyl group, an anthracenyl group, a phenanthryl group, a biphenyl group, a terphenyl group, a quaterphenyl group, a quinquephenyl group, a sexiphenyl group, a triphenylenyl group, a pyrenyl group, a benzofluoranthenyl group, a chrysenyl group, etc., but the embodiment of the present disclosure is not limited thereto.

The term “heterocyclic group” as used herein refers to any functional group or substituent derived from a ring containing at least one of B, O, N, P, Si, or Se as a ring-forming heteroatom. The heterocyclic group includes an aliphatic heterocyclic group and an aromatic heterocyclic group. The aromatic heterocyclic group may be a heteroaryl group. The aliphatic heterocycle and the aromatic heterocycle may be monocyclic or polycyclic.

In the present specification, when the heterocyclic group includes two or more heteroatoms, the two or more heteroatoms may be the same as or different from each other. The number of ring-forming carbon atoms in the heterocyclic group may be 2 to 30, 2 to 20, or 2 to 10.

In the present specification, examples of the heteroaryl group may include a thiophene group, a furan group, a pyrrole group, an imidazole group, a pyridine group, a bipyridine group, a pyrimidine group, a triazine group, a triazole group, an acridyl group, a pyridazine group, a pyrazinyl group, a quinoline group, a quinazoline group, a quinoxaline group, a phenoxazine group, a phthalazine group, a pyrido pyrimidine group, a pyrido pyrazine group, a pyrazino pyrazine group, an isoquinoline group, an indole group, a carbazole group, an N-arylcarbazole group, an N-heteroarylcarbazole group, an N-alkylcarbazole group, a benzoxazole group, a benzimidazole group, a benzothiazole group, a benzocarbazole group, a benzothiophene group, a dibenzothiophene group, a thienothiophene group, a benzofuran group, a phenanthroline group, a thiazole group, an isoxazole group, an oxazole group, an oxadiazole group, a thiadiazole group, a phenothiazine group, a dibenzosilole group, a dibenzofuran group, etc., but the embodiment of the present disclosure is not limited thereto.

In the specification, the above description of the aryl group may be applied to an arylene group except that the arylene group is a divalent group. The above description of the heteroaryl group may be applied to a heteroarylene group except that the heteroarylene group is a divalent group.

In the specification, the silyl group includes an alkylsilyl group and an arylsilyl group. Examples of the silyl group may include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, etc., but the embodiment of the present disclosure is not limited thereto.

In the specification, the thio group may include an alkylthio group and an arylthio group. The thio group may refer to that a sulfur atom is bonded to the alkyl group or the aryl group as defined above. Examples of the thio group may include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, etc., but the embodiment of the present disclosure is not limited thereto.

In the specification, an oxy group may refer to that an oxygen atom is bonded to the alkyl group or the aryl group as defined above. The oxy group may include an alkoxy group and an aryl oxy group. The alkoxy group may be a linear chain, a branched chain or a cyclic chain. The number of carbon atoms in the alkoxy group is not specifically limited, but may be, for example, 1 to 20 or 1 to 10. Examples of the oxy group may include a methoxy group, an ethoxy group, an n-propoxy group, an isopropoxy group, a butoxy group, a pentyloxy group, a hexyloxy group, an octyloxy group, a nonyloxy group, a decyloxy group, a benzyloxy group, etc., but the embodiment of the present disclosure is not limited thereto.

The boron group herein may refer to that a boron atom is bonded to the alkyl group or the aryl group as defined above. The boron group includes an alkyl boron group and an aryl boron group. Examples of the boron group may include a dimethylboron group, a trimethylboron group, a t-butyldimethylboron group, a diphenylboron group, a phenylboron group, etc., but the embodiment of the present disclosure is not limited thereto.

In the specification, the number of carbon atoms in an amine group is not specifically limited, but may be 1 to 30. The amine group may include an alkyl amine group and an aryl amine group. Examples of the amine group may include a methylamine group, a dimethylamine group, a phenylamine group, a diphenylamine group, a naphthylamine group, a 9-methyl-anthracenylamine group, a triphenylamine group, etc., but the embodiment of the present disclosure is not limited thereto.

In the specification, a direct linkage may refer to a single bond.

In some embodiments, “—*” herein refers to a position to be connected.

Hereinafter, embodiments of the present disclosure will be described with reference to the accompanying drawings.

FIG. 1 is a plan view illustrating an embodiment of a display apparatus DD. FIG. 2 is a cross-sectional view of the display apparatus DD of the embodiment. FIG. 2 is a cross-sectional view illustrating a part taken along the line I-I′ of FIG. 1.

The display apparatus DD may include a display panel DP and an optical layer PP disposed on the display panel DP. The display panel DP includes light emitting devices ED-1, ED-2, and ED-3. The display apparatus DD may include a plurality of light emitting devices ED-1, ED-2, and ED-3. The optical layer PP may be disposed on the display panel DP to control reflection of external light at the display panel DP. The optical layer PP may include, for example, a polarization layer and/or a color filter layer. In some embodiments, unlike the configuration illustrated in the drawing, the optical layer PP may not be provided in the display apparatus DD of an embodiment.

A base substrate BL may be disposed on the optical layer PP. The base substrate BL may be a member which provides a base surface on which the optical layer PP is disposed. The base substrate BL may be a glass substrate, a metal substrate, a plastic substrate, etc. However, the embodiment of the present disclosure is not limited thereto, and the base substrate BL may be an inorganic layer, an organic layer, or a composite material layer. In some embodiments, unlike the configuration illustrated, in an embodiment, the base substrate BL may not be provided.

The display apparatus DD according to an embodiment may further include a filling layer. The filling layer may be disposed between a display device layer DP-ED and the base substrate BL. The filling layer may be an organic material layer. The filling layer may include at least one of an acrylic-based resin, a silicone-based resin, or an epoxy-based resin.

The display panel DP may include a base layer BS, a circuit layer DP-CL provided on the base layer BS, and the display device layer DP-ED. The display device layer DP-ED may include a pixel defining film PDL, the light emitting devices ED-1, ED-2, and ED-3 disposed between portions of the pixel defining film PDL, and an encapsulation layer TFE disposed on the light emitting devices ED-1, ED-2, and ED-3.

The base layer BS may be a member which provides a base surface on which the display device layer DP-ED is disposed. The base layer BS may be a glass substrate, a metal substrate, a plastic substrate, etc. However, the embodiment is not limited thereto, and the base layer BS may be an inorganic layer, an organic layer, or a composite material layer.

In an embodiment, the circuit layer DP-CL is disposed on the base layer BS, and the circuit layer DP-CL may include a plurality of transistors. Each of the transistors may include a control electrode, an input electrode, and an output electrode. For example, the circuit layer DP-CL may include a switching transistor and a driving transistor for driving the light emitting devices ED-1, ED-2, and ED-3 of the display device layer DP-ED.

Each of the light emitting devices ED-1, ED-2, and ED-3 may have a structure of a light emitting device ED of an embodiment according to FIGS. 3 to 6, which will be described later. Each of the light emitting devices ED-1, ED-2, and ED-3 may include a first electrode EL1, a hole transport region HTR, emission layers EML-R, EML-G, and EML-B, an electron transport region ETR, and a second electrode EL2.

FIG. 2 illustrates an embodiment in which the emission layers EML-R, EML-G, and EML-B of the light emitting devices ED-1, ED-2, and ED-3 are disposed in openings OH defined in the pixel defining film PDL, and the hole transport region HTR, the electron transport region ETR, and the second electrode EL2 are provided as a common layer in the entire light emitting devices ED-1, ED-2, and ED-3. However, the embodiment of the present disclosure is not limited thereto, and unlike the configuration illustrated in FIG. 2, the hole transport region HTR and the electron transport region ETR in an embodiment may be provided by being patterned inside the openings OH defined in the pixel defining film PDL. For example, the hole transport region HTR, the emission layers EML-R, EML-G, and EML-B, and the electron transport region ETR of the light emitting devices ED-1, ED-2, and ED-3 in an embodiment may be provided by being patterned in an inkjet printing method.

The encapsulation layer TFE may cover the light emitting devices ED-1, ED-2 and ED-3. The encapsulation layer TFE may seal the display device layer DP-ED. The encapsulation layer TFE may be a thin film encapsulation layer. The encapsulation layer TFE may be formed by laminating one layer or a plurality of layers. The encapsulation layer TFE includes at least one insulation layer. The encapsulation layer TFE according to an embodiment may include at least one inorganic film (hereinafter, an encapsulation-inorganic film). The encapsulation layer TFE according to an embodiment may also include at least one organic film (hereinafter, an encapsulation-organic film) and at least one encapsulation-inorganic film.

The encapsulation-inorganic film protects the display device layer DP-ED from moisture/oxygen, and the encapsulation-organic film protects the display device layer DP-ED from foreign substances such as dust particles. The encapsulation-inorganic film may include silicon nitride, silicon oxynitride, silicon oxide, titanium oxide, aluminum oxide, and/or the like, but the embodiment of the present disclosure is not particularly limited thereto. The encapsulation-organic film may include an acrylic-based compound, an epoxy-based compound, and/or the like. The encapsulation-organic film may include a photopolymerizable organic material, but the embodiment of the present disclosure is not particularly limited thereto.

The encapsulation layer TFE may be disposed on the second electrode EL2 and may be disposed filling the opening OH.

Referring to FIGS. 1 and 2, the display apparatus DD may include a non-light emitting region NPXA and light emitting regions PXA-R, PXA-G, and PXA-B. The light emitting regions PXA-R, PXA-G, and PXA-B may be regions in which light generated by the respective light emitting devices ED-1, ED-2, and ED-3 is emitted. The light emitting regions PXA-R, PXA-G, and PXA-B may be spaced apart from each other on a plane (e.g., in a plan view).

Each of the light emitting regions PXA-R, PXA-G, and PXA-B may be a region divided by the pixel defining film PDL. The non-light emitting areas NPXA may be areas between the adjacent light emitting areas PXA-R, PXA-G, and PXA-B, which correspond to the pixel defining film PDL. In some embodiments, in the specification, the light emitting regions PXA-R, PXA-G, and PXA-B may respectively correspond to pixels. The pixel defining film PDL may divide the light emitting devices ED-1, ED-2, and ED-3. The emission layers EML-R, EML-G, and EML-B of the light emitting devices ED-1, ED-2, and ED-3 may be disposed in openings OH defined in the pixel defining film PDL and separated from each other.

The light emitting regions PXA-R, PXA-G, and PXA-B may be divided into a plurality of groups according to the color of light generated from the light emitting devices ED-1, ED-2, and ED-3. In the display apparatus DD of an embodiment illustrated in FIGS. 1 and 2, three light emitting regions PXA-R, PXA-G, and PXA-B, which emit red light, green light, and blue light, respectively, are illustrated as an example. For example, the display apparatus DD of an embodiment may include the red light emitting region PXA-R, the green light emitting region PXA-G, and the blue light emitting region PXA-B that are separated from each other.

In the display apparatus DD according to an embodiment, the plurality of light emitting devices ED-1, ED-2 and ED-3 may be to emit light (e.g., light beams) having wavelengths different from each other. For example, in an embodiment, the display apparatus DD may include a first light emitting device ED-1 that emits red light, a second light emitting device ED-2 that emits green light, and a third light emitting device ED-3 that emits blue light. For example, the red light emitting region PXA-R, the green light emitting region PXA-G, and the blue light emitting region PXA-B of the display apparatus DD may correspond to the first light emitting device ED-1, the second light emitting device ED-2, and the third light emitting device ED-3, respectively.

However, the embodiment of the present disclosure is not limited thereto, and the first to third light emitting devices ED-1, ED-2, and ED-3 may be to emit light (e.g., light beams) in substantially the same wavelength range or at least one light emitting device may be to emit light (e.g., a light beam) in a wavelength range different from the others. For example, the first to third light emitting devices ED-1, ED-2, and ED-3 may all emit blue light.

The light emitting regions PXA-R, PXA-G, and PXA-B in the display apparatus DD according to an embodiment may be arranged in a stripe form. Referring to FIG. 1, the plurality of red light emitting regions PXA-R may be arranged with each other along a second directional axis DR2, the plurality of green light emitting regions PXA-G may be arranged with each other along the second directional axis DR2, and the plurality of blue light emitting regions PXA-B may be arranged with each other along the second directional axis DR2. In some embodiments, the red light emitting region PXA-R, the green light emitting region PXA-G, and the blue light emitting region PXA-B may be alternately arranged in this stated order along a first directional axis DR1.

FIGS. 1 and 2 illustrate that all the light emitting regions PXA-R, PXA-G, and PXA-B have similar areas, but the embodiment of the present disclosure is not limited thereto. Thus, the light emitting regions PXA-R, PXA-G, and PXA-B may have different areas from each other according to the wavelength range of the emitted light. In this case, the areas of the light emitting regions PXA-R, PXA-G, and PXA-B may refer to areas when viewed on a plane defined by the first directional axis DR1 and the second directional axis DR2 (e.g., in a plan view).

In some embodiments, an arrangement form of the light emitting regions PXA-R, PXA-G, and PXA-B is not limited to the configuration illustrated in FIG. 1, and the order in which the red light emitting region PXA-R, the green light emitting region PXA-G, and the blue light emitting region PXA-B are arranged may be provided in one or more suitable combinations according to the characteristics of display quality required in the display apparatus DD. For example, the arrangement form of the light emitting regions PXA-R, PXA-G, and PXA-B may be a pentile (PENTILE®) arrangement form or a diamond (Diamond Pixel™) arrangement form. PENTILE® and Diamond Pixel™ are trademarks of Samsung Display Co., Ltd.

In some embodiments, the areas of the light emitting regions PXA-R, PXA-G, and PXA-B may be different from each other. For example, in an embodiment, the area of the green light emitting region PXA-G may be smaller than that of the blue light emitting region PXA-B, but the embodiment of the present disclosure is not limited thereto.

Hereinafter, FIGS. 3 to 6 are cross-sectional views schematically illustrating light emitting devices according to embodiments. Each of the light emitting devices ED according to embodiments may include a first electrode EL1, a hole transport region HTR, an emission layer EML, an electron transport region ETR, and a second electrode EL2 that are sequentially stacked.

Compared with FIG. 3, FIG. 4 illustrates a cross-sectional view of a light emitting device ED of an embodiment, in which a hole transport region HTR includes a hole injection layer HIL and a hole transport layer HTL, and an electron transport region ETR includes an electron injection layer EIL and an electron transport layer ETL. In addition, compared with FIG. 3, FIG. 5 illustrates a cross-sectional view of a light emitting device ED of an embodiment, in which a hole transport region HTR includes a hole injection layer HIL, a hole transport layer HTL, and an electron blocking layer EBL, and an electron transport region ETR includes an electron injection layer EIL, an electron transport layer ETL, and a hole blocking layer HBL. Compared with FIG. 4, FIG. 6 illustrates a cross-sectional view of a light emitting device ED of an embodiment including a capping layer CPL disposed on a second electrode EL2.

The first electrode EL1 has conductivity (e.g., is a conductor). The first electrode EL1 may be formed of a metal material, a metal alloy, or a conductive compound. The first electrode EL1 may be an anode or a cathode. However, the embodiment of the present disclosure is not limited thereto. In some embodiments, the first electrode EL1 may be a pixel electrode. The first electrode EL1 may be a transmissive electrode, a transflective electrode, or a reflective electrode. The first electrode EL1 may include at least one selected from among Ag, Mg, Cu, Al, Pt, Pd, Au, Ni, Nd, Ir, Cr, Li, Ca, LiF, Mo, Ti, W, In, Sn, and Zn, a compound of two or more selected therefrom, a mixture of two or more selected therefrom, and/or an oxide thereof.

When the first electrode EL1 is the transmissive electrode, the first electrode EL1 may include a transparent metal oxide such as indium tin oxide (ITO), indium zinc oxide (IZO), zinc oxide (ZnO), and/or indium tin zinc oxide (ITZO). When the first electrode EL1 is the transflective electrode or the reflective electrode, the first electrode EL1 may include Ag, Mg, Cu, Al, Pt, Pd, Au, Ni, Nd, Ir, Cr, Li, Ca, LiF/Ca (a stacked structure of LiF and Ca), LiF/Al (a stacked structure of LiF and Al), Mo, Ti, W, and/or a compound and/or mixture thereof (e.g., a mixture of Ag and Mg). In some embodiments, the first electrode EL1 may have a multilayer structure including a reflective film or a transflective film formed of the above-described materials, and a transparent conductive film formed of ITO, IZO, ZnO, ITZO, etc. For example, the first electrode EL1 may have a three-layer structure of ITO/Ag/ITO, but the embodiment of the present disclosure is not limited thereto. In addition, the embodiment of the present disclosure is not limited thereto, and the first electrode EL1 may include one or more of the above-described metal materials, combinations of at least two metal materials selected from among the above-described metal materials, oxides of the above-described metal materials, and/or the like. The thickness of the first electrode EL1 may be from about 700 Å to about 10,000 Å. For example, the thickness of the first electrode EL1 may be from about 1,000 Å to about 3,000 Å.

The hole transport region HTR is provided on the first electrode EL1. The hole transport region HTR may include at least one of a hole injection layer HIL, a hole transport layer HTL, a buffer layer or an emission-auxiliary layer, or an electron blocking layer EBL. The thickness of the hole transport region HTR may be, for example, from about 50 Å to about 15,000 Å.

The hole transport region HTR may have a single layer formed of a single material, a single layer formed of a plurality of different materials, or a multilayer structure including a plurality of layers formed of a plurality of different materials.

For example, the hole transport region HTR may have a single layer structure of the hole injection layer HIL or the hole transport layer HTL, or may have a single layer structure formed of a hole injection material and a hole transport material. In some embodiments, the hole transport region HTR may have a single layer structure formed of a plurality of different materials, or a structure in which a hole injection layer HIL/a hole transport layer HTL, a hole injection layer HIL/a hole transport layer HTL/a buffer layer, a hole injection layer HIL/a buffer layer, a hole transport layer HTL/a buffer layer, or a hole injection layer HIL/a hole transport layer HTL/a electron blocking layer EBL, that are stacked in the respective stated order from the first electrode EL1, but the embodiment of the present disclosure is not limited thereto.

The hole transport region HTR may be formed utilizing one or more suitable methods such as a vacuum deposition method, a spin coating method, a cast method, a Langmuir-Blodgett (LB) method, an inkjet printing method, a laser printing method, and/or a laser induced thermal imaging (LITI) method.

The hole transport region HTR may include a compound represented by Formula H-20:

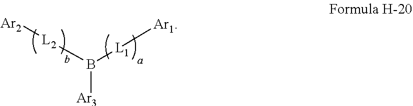

In Formula H-20, L₁ and L₂ may each independently be a direct linkage, a substituted or unsubstituted arylene group having 6 to 30 ring-forming carbon atoms, or a substituted or unsubstituted heteroarylene group having 2 to 30 ring-forming carbon atoms. a and b may each independently be an integer of 0 to 10. In some embodiments, when a or b is an integer of 2 or greater, a plurality of Li's and L₂'s may each independently be a substituted or unsubstituted arylene group having 6 to 30 ring-forming carbon atoms, or a substituted or unsubstituted heteroarylene group having 2 to 30 ring-forming carbon atoms.

In Formula H-20, Aᵣ₁ and Ar₂ may each independently be a substituted or unsubstituted aryl group having 6 to 30 ring-forming carbon atoms, or a substituted or unsubstituted heteroaryl group having 2 to 30 ring-forming carbon atoms. In some embodiments, in Formula H-20, Ar₃ may be a substituted or unsubstituted aryl group having 6 to 30 ring-forming carbon atoms, or a substituted or unsubstituted heteroaryl group having 2 to 30 ring-forming carbon atoms.

The compound represented by Formula H-20 may be a monoamine compound (e.g., a compound including a single amine group). In some embodiments, the compound represented by Formula H-20 may be a diamine compound in which at least one from among An to Ar₃ includes the amine group as a substituent. In some embodiments, the compound represented by Formula H-20 may be a carbazole-based compound including a substituted or unsubstituted carbazole group in at least one of Ar₁ or Ar₂, or a fluorene-based compound including a substituted or unsubstituted fluorene group in at least one of Ar₁ or Ar₂.

The compound represented by Formula H-20 may be represented by any one from among the compounds of Compound Group H. However, the compounds listed in Compound Group H are examples, and the compounds represented by Formula H-20 are not limited to those represented by Compound Group H:

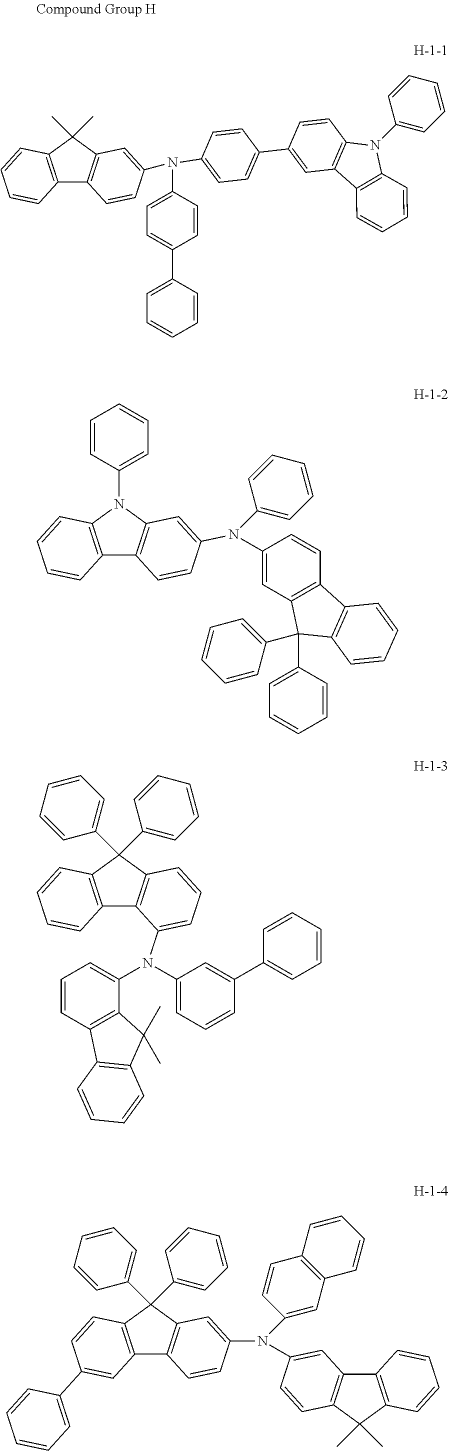

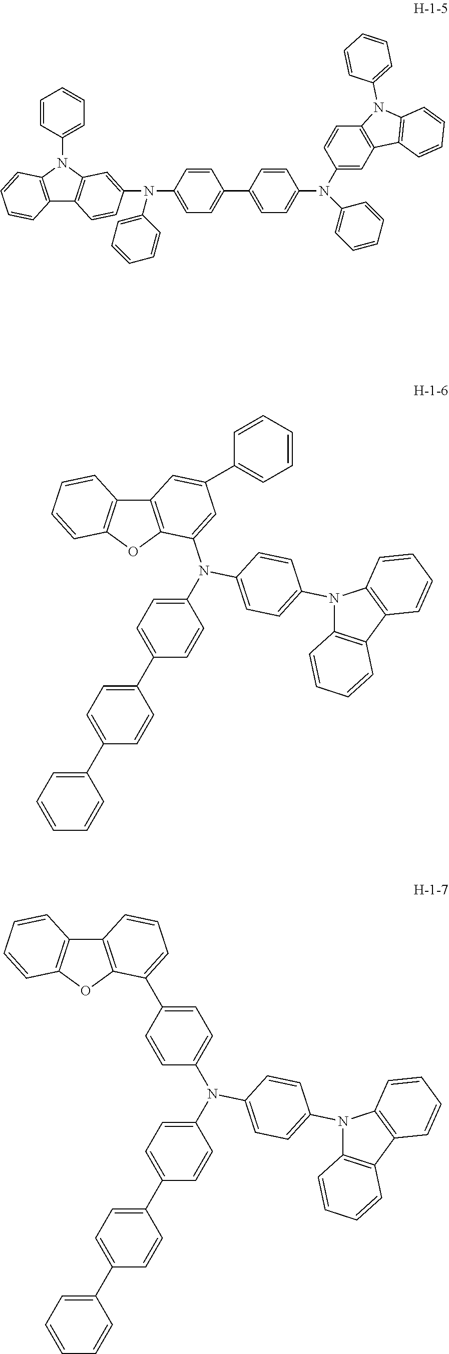 [SMILES: c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1 ; c1ccc(-c2ccc(-c3ccc(N(c4ccc(-c5cccc6c5oc5ccccc56)cc4)c4ccc(-n5c6ccccc6c6ccccc65)cc4)cc3)cc2)cc1]

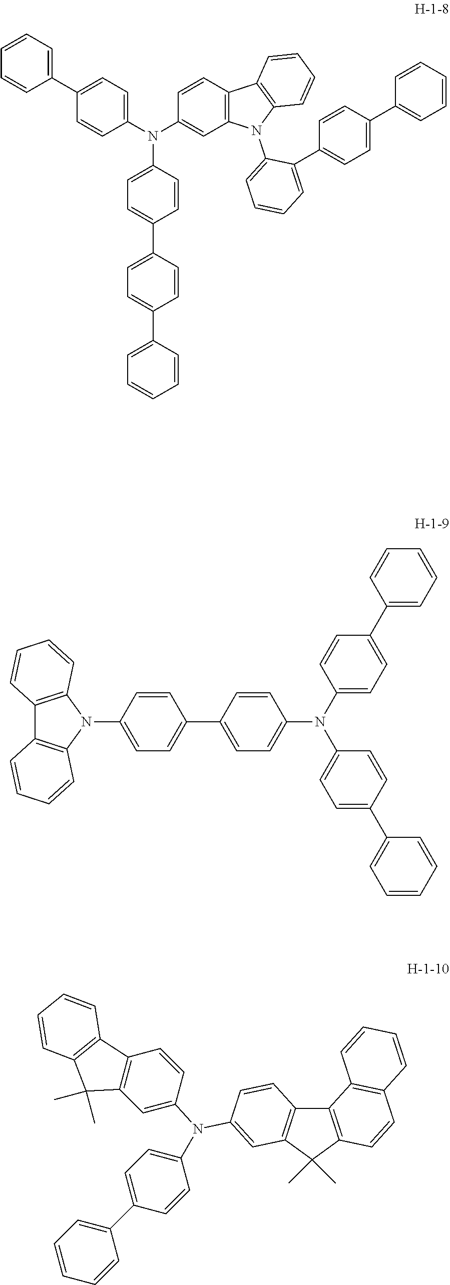 [SMILES: c1ccc(-c2ccc(-c3ccc(N(c4ccc(-c5ccccc5)cc4)c4ccc5c6ccccc6n(-c6ccccc6-c6ccc(-c7ccccc7)cc6)c5c4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2ccccc2-c2ccc(N(c3ccc(-c4ccccc4)cc3)c3ccc4c(c3)C(C)(C)c3ccc5ccccc5c3-4)cc21]

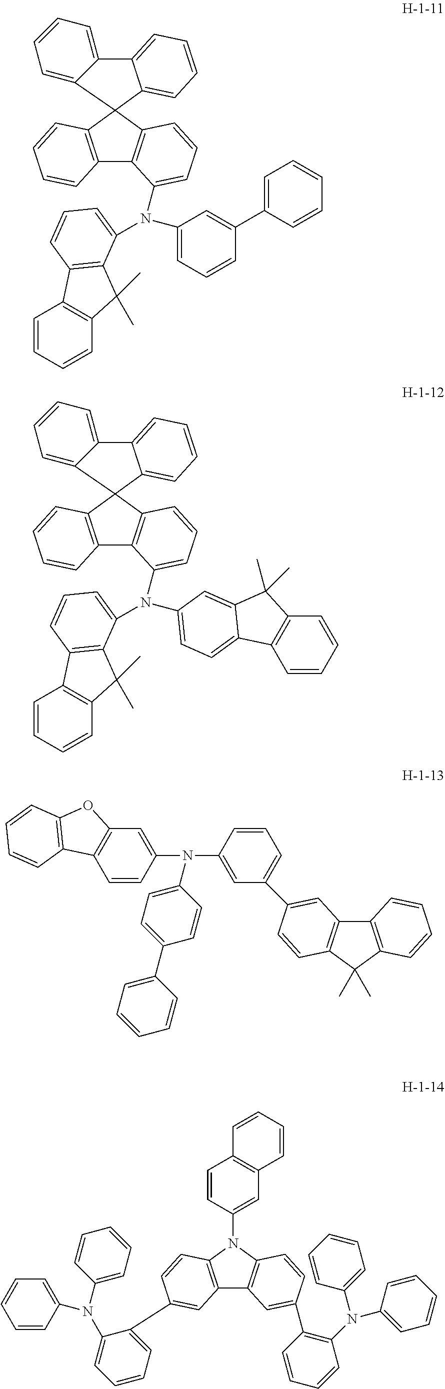

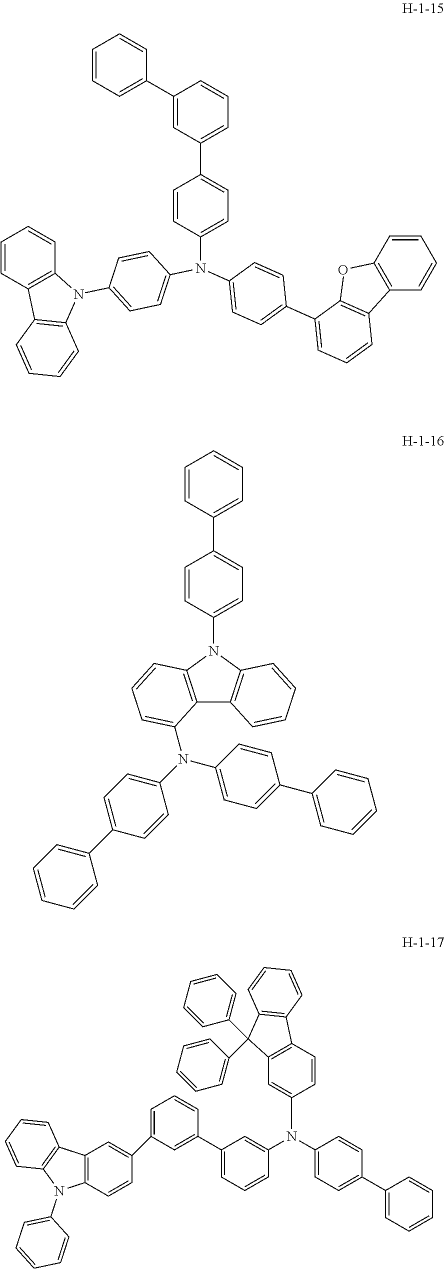 [SMILES: c1ccc(-c2cccc(-c3ccc(N(c4ccc(-c5cccc6c5oc5ccccc56)cc4)c4ccc(-n5c6ccccc6c6ccccc65)cc4)cc3)c2)cc1 ; c1ccc(-c2ccc(N(c3ccc(-c4ccccc4)cc3)c3cccc4c3c3ccccc3n4-c3ccc(-c4ccccc4)cc3)cc2)cc1 ; c1ccc(-c2ccc(N(c3cccc(-c4cccc(-c5ccc6c(c5)c5ccccc5n6-c5ccccc5)c4)c3)c3ccc4c(c3)C(c3ccccc3)(c3ccccc3)c3ccccc3-4)cc2)cc1]

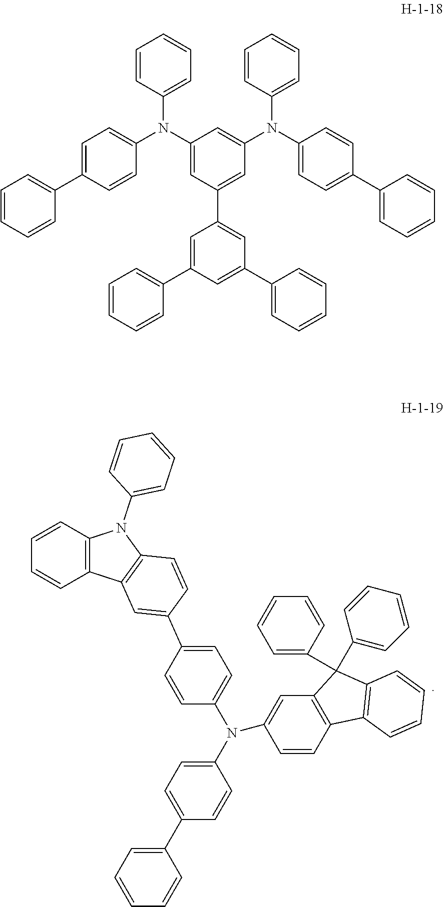 [SMILES: c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

The hole transport region HTR may include a phthalocyanine compound such as copper phthalocyanine;
N¹,N¹′-([1,1′-biphenyl]-4,4′-diyl)bis(N¹-phenyl-N⁴,N⁴-di-m-tolylbenzene-1,4-diamine) (DNTPD), 4,4′,4″-[tris(3-methylphenyl)phenylamino] triphenylamine (m-MTDATA),
4,4′,4″-tris(N,N-diphenylamino)triphenylamine (TDATA),
4,4′,4″-tris[N(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS),
N,N′-di(naphthalene-I-yl)-N,N′-diphenyl-benzidine (NPB), triphenylamine-containing polyetherketone (TPAPEK), 4-isopropyl-4′-methyldiphenyliodonium [tetrakis(pentafluorophenyl)borate], dipyrazino[2,3-f:
2′,3′-h]quinoxaline-2,3,6,7,10,11-hexacarbonitrile (HATCN), etc.

The hole transport region HTR may include a carbazole-based derivative such as N-phenyl carbazole and/or polyvinyl carbazole, a fluorene-based derivative, a triphenylamine-based derivative such as
N,N′-bis(3-methylphenyl)-N,N′-diphenyl-[1,1-biphenyl]-4,4′-diamine (TPD) and/or
4,4′,4″-tris(N-carbazolyl)triphenylamine (TCTA),
N,N′-di(naphthalene-I-yl)-N,N′-diphenyl-benzidine (NPB), 4,4′-cyclohexylidene bis[N,N-bis(4-methylphenyl]benzenamine] (TAPC),
4,4′-bis[N,N′-(3-tolyl)amino]-3,3′-dimethylbiphenyl (HMTPD),
1,3-bis(N-carbazolyl)benzene (mCP), etc.

In some embodiments, the hole transport region HTR may include
9-(4-tert-butylphenyl)-3,6-bis(triphenylsilyl)-9H-carbazole (CzSi),
9-phenyl-9H-3,9′-bicarbazole (CCP), 1,3-bis(1,8-dimethyl-9H-carbazol-9-yl)benzene (mDCP), etc.

The hole transport region HTR may include the above-described compounds of the hole transport region HTR in at least one of a hole injection layer HIL, a hole transport layer HTL, or an electron blocking layer EBL.

The thickness of the hole transport region HTR may be from about 100 Å to about 10,000 Å, for example, from about 100 Å to about 5,000 Å. When the hole transport region HTR includes the hole injection layer HIL, the hole injection layer HIL may have, for example, a thickness of about 30 Å to about 1,000 Å. When the hole transport region HTR includes the hole transport layer HTL, the hole transport layer HTL may have a thickness of about 250 Å to about 1,000 Å. For example, when the hole transport region HTR includes the electron blocking layer EBL, the electron blocking layer EBL may have a thickness of about 10 Å to about 1,000 Å. When the thicknesses of the hole transport region HTR, the hole injection layer HIL, the hole transport layer HTL and the electron blocking layer EBL satisfy the above-described ranges, satisfactory hole transport properties may be achieved without a substantial increase in driving voltage.

The hole transport region HTR may further include a charge generating material to increase conductivity in addition to the above-described materials. The charge generating material may be dispersed uniformly or non-uniformly in the hole transport region HTR. The charge generating material may be, for example, a p-dopant. The p-dopant may include at least one of a halogenated metal compound, a quinone derivative, a metal oxide, or a cyano group-containing compound, but the embodiment of the present disclosure is not limited thereto. For example, the p-dopant may include a metal halide compound such as CuI and/or RbI, a quinone derivative such as tetracyanoquinodimethane (TCNQ) and/or
2,3,5,6-tetrafluoro-7,7′8,8-tetracyanoquinodimethane (F4-TCNQ), a metal oxide such as tungsten oxide and/or molybdenum oxide, a cyano group-containing compound such as dipyrazino[2,3-f: 2′,3′-h] quinoxaline-2,3,6,7,10,11-hexacarbonitrile (HATCN) and/or
4-[[2,3-bis[cyano-(4-cyano-2,3,5,6-tetrafluorophenyl)methylidene]cyclopropylidene]-cya nomethyl]-2,3,5,6-tetrafluorobenzonitrile (NDP9), etc., but the embodiment of the present disclosure is not limited thereto.

As described, the hole transport region HTR may further include at least one of the buffer layer or the electron blocking layer EBL in addition to the hole injection layer HIL and the hole transport layer HTL. The buffer layer may compensate for a resonance distance according to the wavelength of light emitted from the emission layer EML and may thus increase light emission efficiency. A material that may be contained in the hole transport region HTR may be utilized as a material to be contained in the buffer layer. The electron blocking layer EBL is a layer that serves to prevent or reduce the electron injection from the electron transport region ETR to the hole transport region HTR.

The emission layer EML is provided on the hole transport region HTR. The emission layer EML may have a thickness of, for example, about 100 Å to about 1,000 Å or about 100 Å to about 300 Å. The emission layer EML may have a single layer formed of a single material, a single layer formed of a plurality of different materials, or a multilayer structure having a plurality of layers formed of a plurality of different materials.

The emission layer EML in the light emitting device ED according to an embodiment may include a fused polycyclic compound of an embodiment. In an embodiment, the emission layer EML may include the fused polycyclic compound of an embodiment as a dopant. The fused polycyclic compound of an embodiment may be a dopant material of the emission layer EML. In the specification, the fused polycyclic compound of an embodiment, which will be described later, may be referred to as a first compound.

The fused polycyclic compound of an embodiment may include an indolocarbazole moiety, and have a structure in which a first carbazole group is fused at one benzene ring from among the benzene rings constituting the indolocarbazole moiety. In the specification, the term “indolocarbazole” may refer to an aromatic heterocycle forming a structure in which three benzene rings are fused around one nitrogen atom. For example, the indolocarbazole may have a structure in which first to third benzene rings are fused around a first nitrogen atom, and the first benzene ring and the third benzene ring may form a symmetric structure around the second benzene ring.

The fused polycyclic compound of an embodiment may have a structure in which the first carbazole group is fused at the first benzene ring of the indolocarbazole moiety. The first carbazole group may include a second nitrogen atom, and include a structure in which a fourth and fifth benzene rings are fused around the second nitrogen atom. The first carbazole group may be fused to the indolocarbazole moiety via the second nitrogen atom and a carbon atom at the ortho-position with respect to a carbon atom linked to the second nitrogen atom. For example, the second nitrogen atom and the carbon atom at position 1, or the second nitrogen atom and the carbon atom at position 8 of the first carbazole group may be linked to the first benzene ring of the indolocarbazole group. In this case, the first nitrogen atom of the indolocarbazole group and the second nitrogen atom of the first carbazole group may be linked at the para-position with respect to the first benzene ring. In the specification, a structure in which the first carbazole group is fused to the indolocarbazole moiety may be referred to as the “fused ring core.”

The numbering of carbon atoms of the first carbazole group is as shown in Formula C:

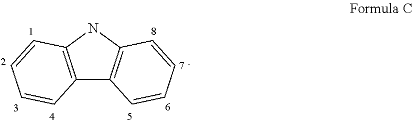 [SMILES: c1ccc2c(c1)[nH]c1ccccc12]

The fused polycyclic compound of an embodiment may include a structure in which a sixth benzene ring is fused to a fused ring core via a sulfur atom. For example, the sixth benzene ring may be fused to at least one from among (e.g., one selected from among) the second and third benzene rings of the indolocarbazole moiety of the fused ring core and (e.g., one selected from among) the fourth and fifth benzene rings of the first carbazole group, and in this case, the sixth benzene ring may be fused via a sulfur atom. For example, the fused polycyclic compound of an embodiment may have a structure in which the sixth benzene ring is fused to at least one from among the second to fifth benzene rings constituting the fused ring core via a sulfur atom.

The fused polycyclic compound of an embodiment may be represented by Formula 1:

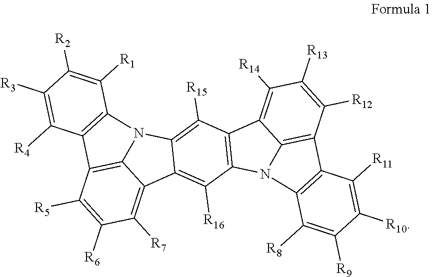

The fused polycyclic compound represented by Formula 1 of an embodiment may include a structure in which the first carbazole group is fused to the indolocarbazole moiety. The indolocarbazole moiety may be fused at the carbon atom at position 5 and the carbon atom at position 6 with the first carbazole group, or may be fused at the carbon atom at position 9 and the carbon atom at position 10 with the first carbazole group. In this case, the nitrogen atom of the indolocarbazole moiety and the nitrogen atom of the first carbazole group may be bonded at the para-position. For example, the nitrogen atom of the first carbazole group may be linked at the carbon atom at position 5 or the carbon atom at position 10 of the indolocarbazole moiety. The carbon number of the indolocarbazole moiety is as shown in Formula C1:

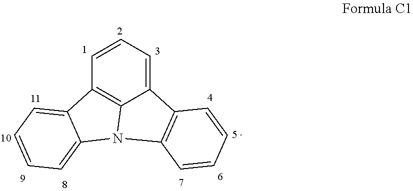 [SMILES: c1ccc2c(c1)c1cccc3c4ccccc4n2c13]

In Formula 1, R₁ to R₁₆ may each independently be a hydrogen atom, a deuterium atom, a halogen atom, a cyano group, a substituted or unsubstituted silyl group, a substituted or unsubstituted amine group, a substituted or unsubstituted oxy group, a substituted or unsubstituted thio group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. In some embodiments, each of R₁ to R₁₆ may be bonded to an adjacent group to form a ring. For example, R₁ to R₁₆ may each independently be a hydrogen atom, a deuterium atom, a substituted or unsubstituted phenyl group, a substituted or unsubstituted biphenyl group, a substituted or unsubstituted terphenyl group, or a substituted or unsubstituted carbazole group.

In Formula 1, at least one pair from among adjacent pairs of R₁ to R₁₄ is positions to which a substituent represented by Formula 2 is fused. For example, the at least one pair of adjacent groups selected from among R₁ to R₁₄ are bonded to each other to form the substituent represented by Formula 2.

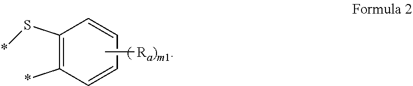

In Formula 2, —* is a position which is fused to any adjacent one pair from among R₁ to R₁₄ in Formula 1 above (e.g., a bonding site to a core of Formula 1).

In Formula 2, Rₐ is a hydrogen atom, a deuterium atom, a halogen atom, a cyano group, a substituted or unsubstituted silyl group, a substituted or unsubstituted amine group, a substituted or unsubstituted oxy group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. In some embodiments, Rₐ may be bonded to an adjacent group to form a ring.

In Formula 2, m1 is an integer of 0 to 4. In Formula 2, when m1 is 0, the fused polycyclic compound of an embodiment may not be substituted with Rₐ. In Formula 2, the case where m1 is 4 and Rₐ's are all hydrogen atoms may be the same as the case where m1 is 0 in Formula 2. When m1 is an integer of 2 or more, a plurality of Rₐ's may all be the same, or at least one from among the plurality of Rₐ's may be different from the others.

In an embodiment, the fused polycyclic compound represented by Formula 1 may be represented by Formula 3-1 or Formula 3-2:

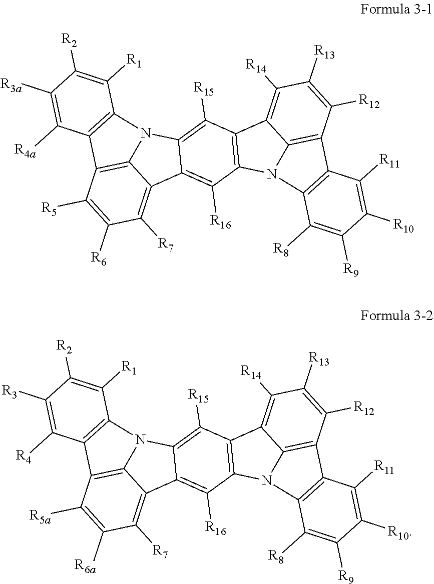

Formula 3-1 and Formula 3-2 each represent an embodiment that the fused position of the substituent represented by Formula 2 is specified in Formula 1.

In Formula 3-1, R₃ₐ and R₄ₐ are positions to which the substituent represented by Formula 2 is fused (e.g., R₃ₐ and R₄ₐ are bonded to each other to form the substituent represented by Formula 2). In Formula 3-1, the substituent represented by Formula 2 may be fused to the positions of R₃ₐ and R₄ₐ. In Formula 3-1, at least one pair from among adjacent pairs of R₁, R₂, and R₅ to R₁₄ may (e.g., also) be positions to which the substituent represented by Formula 2 is fused. However, the embodiment of the present disclosure is not limited thereto, and at the positions other than R₃ₐ and R₄ₐ, the substituent represented by Formula 2 may not be further fused.

In Formula 3-2, R₅ₐ and R₆ₐ are positions to which the substituent represented by Formula 2 is fused (e.g., R₅ₐ and R₆ₐ are bonded to each other to form the substituent represented by Formula 2). In Formula 3-2, the substituent represented by Formula 2 may be fused to the positions of R₅ₐ and R₆ₐ. In Formula 3-2, at least one pair from among adjacent pairs of R₁ to R₄ and R₇ to R₁₄ may (e.g., also) be positions to which the substituent represented by Formula 2 is fused. However, the embodiment of the present disclosure is not limited thereto, and at the positions other than R₅ₐ and R₆ₐ, the substituent represented by Formula 2 may not be further fused.

In Formula 3-1 and Formula 3-2, the same as described in Formula 1 may be applied to R₁ to R₁₆.

In an embodiment, the fused polycyclic compound represented by Formula 1 may be represented by Formula 4-1 or Formula 4-2:

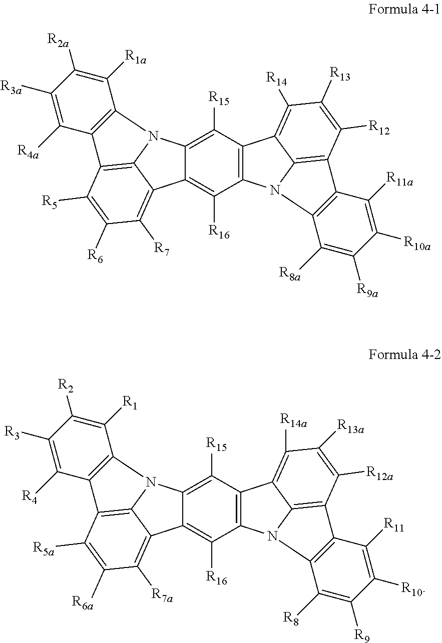

Formula 4-1 and Formula 4-2 each represent an embodiment that the fused position of the substituent represented by Formula 2 is specified in Formula 1.

In Formula 4-1 and Formula 4-2, R₁ₐ to R₁₄ₐ may each independently be a hydrogen atom, a deuterium atom, a halogen atom, a cyano group, a substituted or unsubstituted silyl group, a substituted or unsubstituted amine group, a substituted or unsubstituted oxy group, a substituted or unsubstituted thio group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. In some embodiments, each of R₁ₐ to R₁₄ₐ may be bonded to an adjacent group to form a ring. For example, R₁ₐ to R₁₄ₐ may each independently be a hydrogen atom, a deuterium atom, a substituted or unsubstituted phenyl group, a substituted or unsubstituted biphenyl group, a substituted or unsubstituted terphenyl group, or a substituted or unsubstituted carbazole group.

In Formula 4-1, one adjacent pair selected from among R₁ₐ to R₄ₐ and one adjacent pair selected from among R₈ₐ to R₁₁ₐ may each be positions to which the substituent represented by Formula 2 is fused (e.g., one pair of adjacent groups selected from among R₁ₐ to R₄ₐ are bonded to each other to form the substituent represented by Formula 2, and one pair of adjacent groups selected from among R₈ₐ to R₁₁ₐ are bonded to each other to form the substituent represented by Formula 2).

In Formula 4-2, one adjacent pair selected from among R₅ₐ to R₇ₐ and one adjacent pair selected from among R₁₂ₐ to R₁₄ₐ may each be positions to which the substituent represented by Formula 2 is fused.

In Formula 4-1 and Formula 4-2, the same as described in Formula 1 may be applied to R₁ to R₁₆.

In an embodiment, the fused polycyclic compound represented by Formula 1 may be represented by Formula 5-1 or Formula 5-2:

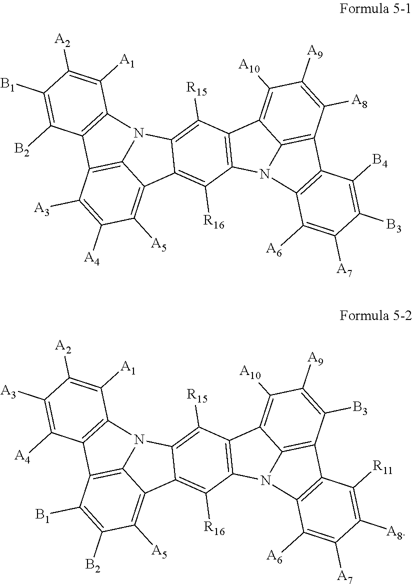

Formula 5-1 and Formula 5-2 each represent an embodiment that the fused number and position of substituents represented by Formula 2 are specified in Formula 1.

In Formula 5-1 and Formula 5-2, A₁ to A₁₀ may each independently be a hydrogen atom, a deuterium atom, a halogen atom, a cyano group, a substituted or unsubstituted silyl group, a substituted or unsubstituted amine group, a substituted or unsubstituted oxy group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. In some embodiments, each of A₁ to A₁₀ may be bonded to an adjacent group to form a ring. For example, A₁ to A₁₀ may each independently be a hydrogen atom, a deuterium atom, a substituted or unsubstituted phenyl group, a substituted or unsubstituted biphenyl group, a substituted or unsubstituted terphenyl group, or a substituted or unsubstituted carbazole group.

In Formula 5-1 and Formula 5-2, B₁ and B₂, and B₃ and B₄ may each be positions to which the substituent represented by Formula 2 is fused (e.g., B₁ and B₂ are bonded to each other to form the substituent represented by Formula 2, and B₃ and B₄ are bonded to each other to form the substituent represented by Formula 2). The fused polycyclic compounds represented by Formula 5-1 and Formula 5-2 may include two substituents represented by Formula 2, one of the two substituents represented by Formula 2 may be fused at B₁ and B₂ positions, and the other one may be fused at B₃ and B₄ positions.

In Formula 5-1 and Formula 5-2, the same as described in Formula 1 may be applied to R₁₅ and R₁₆.

In an embodiment, the fused polycyclic compound represented by Formula 1 may be represented by any one from among Formula 6-1 to Formula 6-4:

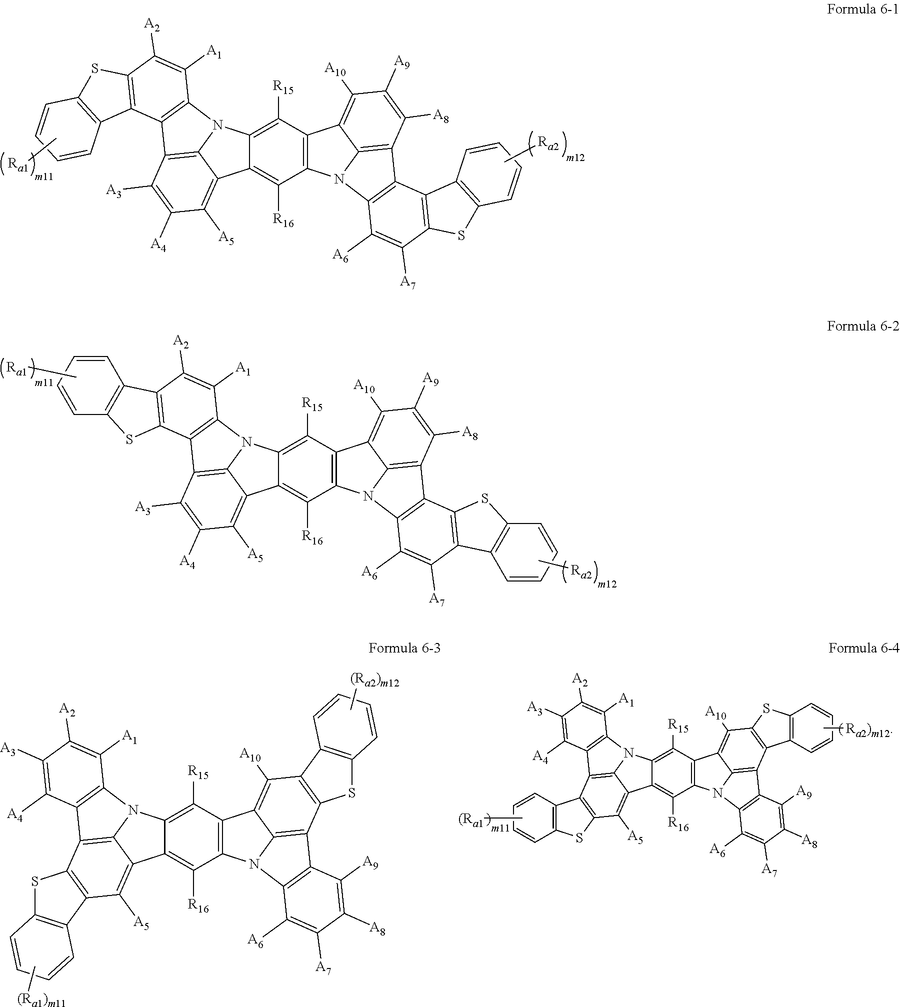

Formula 6-1 to Formula 6-4 each represent an embodiment that substituents represented by Formula 2 are fused in Formula 1.

In Formula 6-1 to Formula 6-4, Rₐ₁ and Rₐ₂ may each independently be a hydrogen atom, a deuterium atom, a halogen atom, a cyano group, a substituted or unsubstituted silyl group, a substituted or unsubstituted amine group, a substituted or unsubstituted oxy group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. In some embodiments, each of Rₐ₁ and Rₐ₂ may be bonded to an adjacent group to form a ring. For example, Rₐ₁ and Rₐ₂ may each independently be a hydrogen atom or a deuterium atom.

In Formula 6-1 to Formula 6-4, m11 and m12 may each independently be an integer of 0 to 4. When m11 and/or m12 is 0, the fused polycyclic compound of an embodiment may not be substituted with Rₐ₁ and/or Rₐ₂. The case where m11 and/or m12 is 4 and Rₐ₁'s and Rₐ₂'s each are hydrogen atoms may be the same as the case where m11 and/or m12 is 0. When m11 and/or m12 is an integer of 2 or more, a plurality of Rₐ₁'s and/or Rₐ₂'s may each be the same or at least one from among the plurality of Rₐ₁'s and Rₐ₂'s may be different from the others.

In Formula 6-1 to Formula 6-4, the same as described in Formula 1, Formula 5-1, and Formula 5-2 may be applied to R₁₅, R₁₆, and A₁ to A₁₀.

In an embodiment, the fused polycyclic compound represented by Formula 1 may be represented by any one from among Formula 7-1 to Formula 7-4:

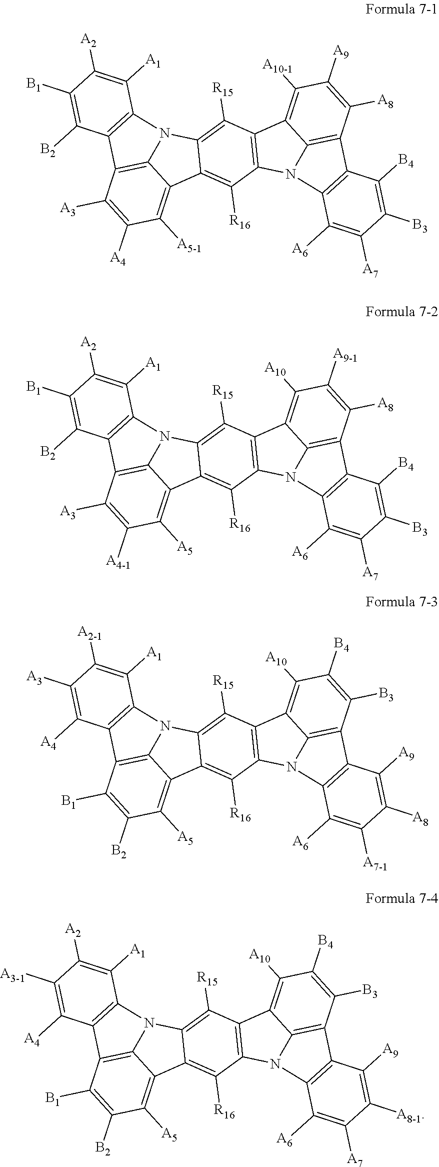

Formula 7-1 to Formula 7-4 each represent an embodiment that the types (kinds) of substituents represented by A₂ to A₅ and A₇ to A₁₀ are specified in Formula 5-1 and Formula 5-2.

In Formula 7-1 to Formula 7-4, A₂-1 to A₅₋₁ and A₇₋₁ to A₁₀₋₁ may each independently be a substituted or unsubstituted aryl group having 6 to 30 ring-forming carbon atoms, or a substituted or unsubstituted heteroaryl group having 2 to 30 ring-forming carbon atoms. For example, A₂₋₁ to A₅₋₁ and A₇-1 to A₁₀₋₁ may each independently be a substituted or unsubstituted phenyl group, a substituted or unsubstituted biphenyl group, a substituted or unsubstituted terphenyl group, or a substituted or unsubstituted carbazole group.

In Formula 7-1 to Formula 7-4, the same as described in Formula 1, Formula 5-1, and Formula 5-2 may be applied to R₁₅, R₁₆, A₁ to A₁₀, and B₁ to B₄.

In an embodiment, A₂₋₁ to A₅₋₁ and A₇₋₁ to A₁₀₋₁ in Formula 7-1 to Formula 7-4 may each independently be represented by any one from among Formula 8-1 to Formula 8-4:

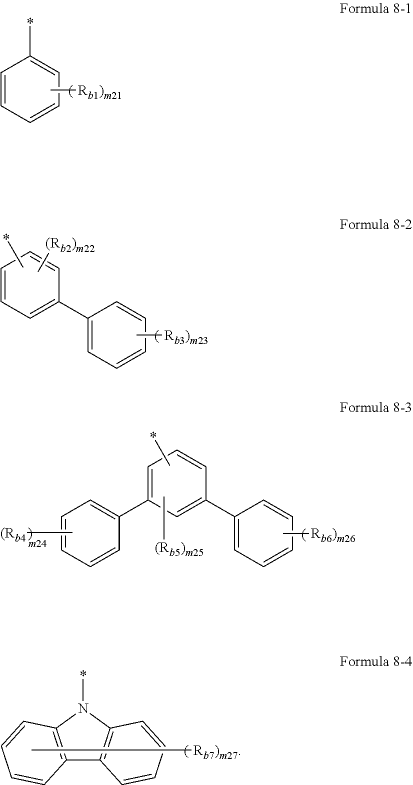

In Formula 8-1 to Formula 8-4, R_{b1} to R_{b7} may each independently be a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. For example, R_{b1} to R_{b7} may each independently be a hydrogen atom, a deuterium atom, a substituted or unsubstituted t-butyl group, a substituted or unsubstituted phenyl group, or a substituted or unsubstituted carbazole group.

In Formula 8-1 and Formula 8-3, m21, m23, m24, and m26 may each independently be an integer of 0 to 5. When m21, m23, m24, and/or m26 is 0, the fused polycyclic compound of an embodiment may not be substituted with R_{b1}, R_{b3}, R_{b4}, and/or R_{b6}. The case where m21, m23, m24, and/or m26 is 5 and R_{b1}'s, R_{b3}'s, R_{b4}'s, and/or R_{b6}'s are each hydrogen atoms may be the same as the case where m21, m23, m24, and/or m26 is 0. When m21, m23, m24, and/or m26 is an integer of 2 or more, a plurality of R_{b1}'s, R_{b3}'s, R_{b4}'s and/or R_{b6}'s each may be the same or at least one from among the plurality of R_{b1}'s, R_{b3}'s, R_{b4}'s and/or R_{b6}'s may be different from the others.

In Formula 8-2, m22 is an integer of 0 to 4. In Formula 8-2, when m22 is 0, the fused polycyclic compound of an embodiment may not be substituted with R_{b2}. In Formula 8-2, the case where m22 is 4 and R_{b2}'s are all hydrogen atoms may be the same as the case where m22 is 0 in Formula 8-2. When m22 is an integer of 2 or more, a plurality of R_{b2}'s may be all the same or at least one from among the plurality of R_{b2}'s may be different from the others.

In Formula 8-3, m25 is an integer of 0 to 3. In Formula 8-3, when m25 is 0, the fused polycyclic compound of an embodiment may not be substituted with R_{b5}. In Formula 8-3, the case where m25 is 3 and R_{b5}'s are all hydrogen atoms may be the same as the case where m25 is 0 in Formula 8-3. When m25 is an integer of 2 or more, a plurality of R_{b5}'s may be all the same or at least one from among the plurality of R_{b5}'s may be different from the others.

In Formula 8-4, m27 is an integer of 0 to 8. In Formula 8-4, when m27 is 0, the fused polycyclic compound of an embodiment may not be substituted with R_{b7}. In Formula 8-4, the case where m27 is 8 and R_{b7}'s are all hydrogen atoms may be the same as the case where m27 is 0 in Formula 8-4. When m27 is an integer of 2 or more, a plurality of R_{b7}'s may all be the same, or at least one of the plurality of R_{b7}'s may be different from the others.

In an embodiment, the fused polycyclic compound represented by Formula 1 may be represented by Formula 9:

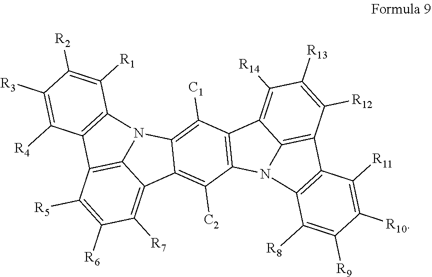

Formula 9 represents an embodiment that the types (kinds) of substituents represented by R₁₅ and R₁₆ are specified in Formula 1.

In Formula 9, C1 and C₂ may each independently be a hydrogen atom or a deuterium atom.

In Formula 9, the same as described in Formula 1 may be applied to R₁ to R₁₄.

The fused polycyclic compound of an embodiment may be any one from among the compounds represented by Compound Group 1. The light emitting device ED of an embodiment may include at least one fused polycyclic compound from among the compounds represented by Compound Group 1 in the emission layer EML.

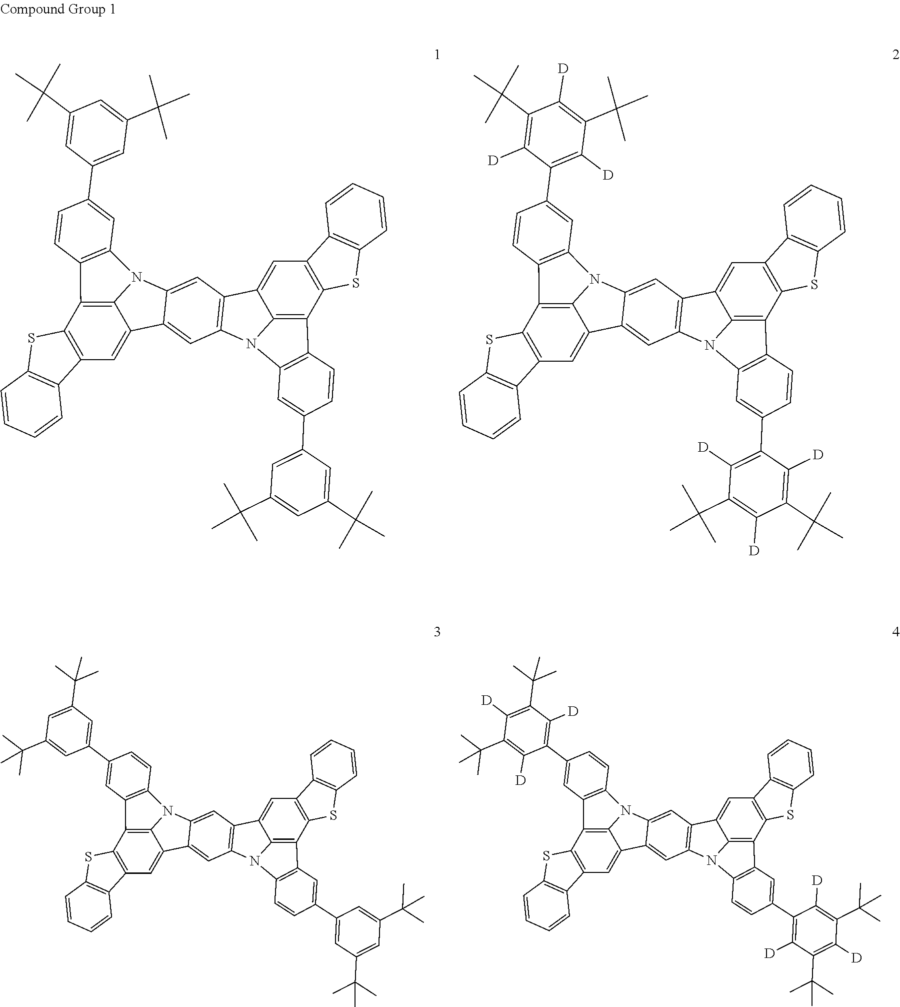

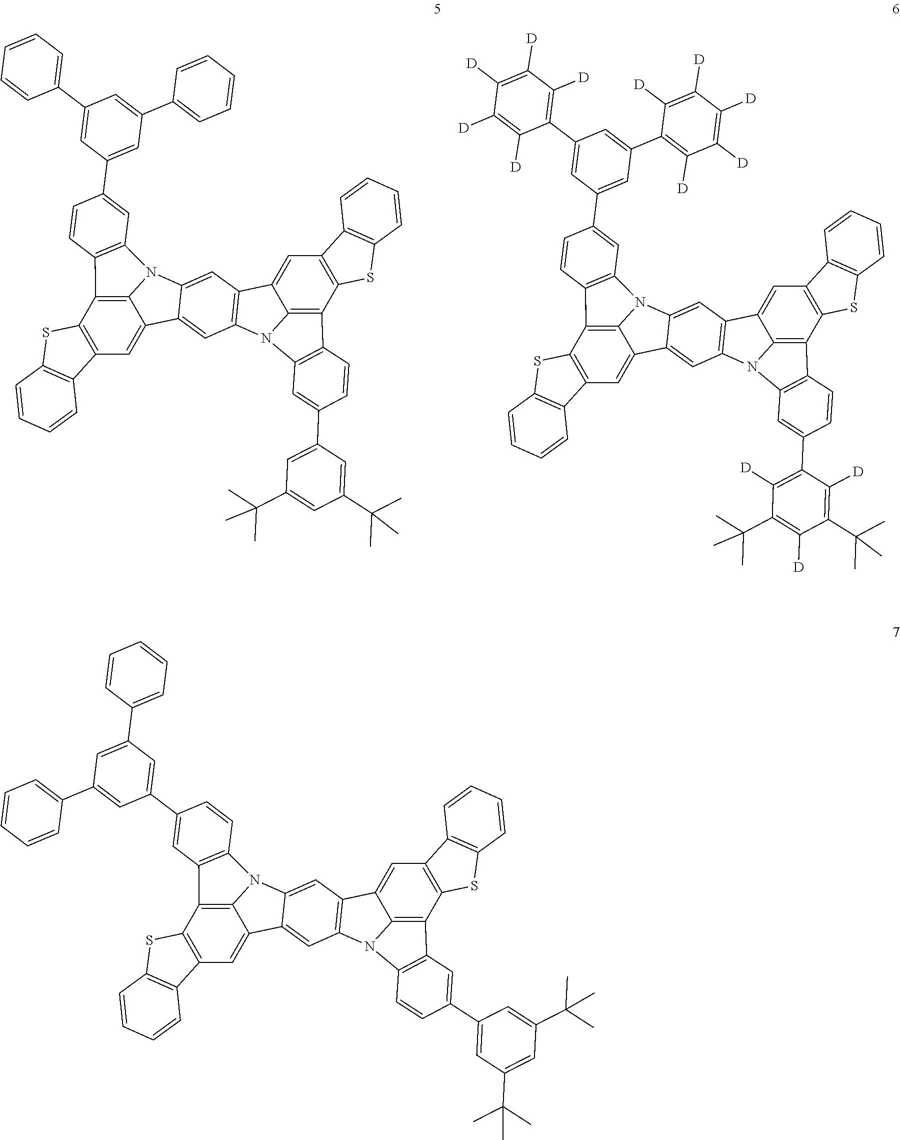 [SMILES: CC(C)(C)c1cc(-c2ccc3c4c5sc6ccccc6c5cc5c6cc7c(cc6n(c3c2)c54)c2cc3c4ccccc4sc3c3c4ccc(-c5cc(-c6ccccc6)cc(-c6ccccc6)c5)cc4n7c23)cc(C(C)(C)C)c1 ; [2H]c1c([2H])c([2H])c(-c2cc(-c3ccc4c5c6sc7ccccc7c6cc6c7cc8c(cc7n(c4c3)c65)c3cc4c5ccccc5sc4c4c5ccc(-c6c([2H])c(C(C)(C)C)c([2H])c(C(C)(C)C)c6[2H])cc5n8c34)cc(-c3c([2H])c([2H])c([2H])c([2H])c3[2H])c2)c([2H])c1[2H] ; CC(C)(C)c1cc(-c2ccc3c(c2)c2c4sc5ccccc5c4cc4c5cc6c(cc5n3c42)c2cc3c4ccccc4sc3c3c4cc(-c5cc(-c7ccccc7)cc(-c7ccccc7)c5)ccc4n6c23)cc(C(C)(C)C)c1]

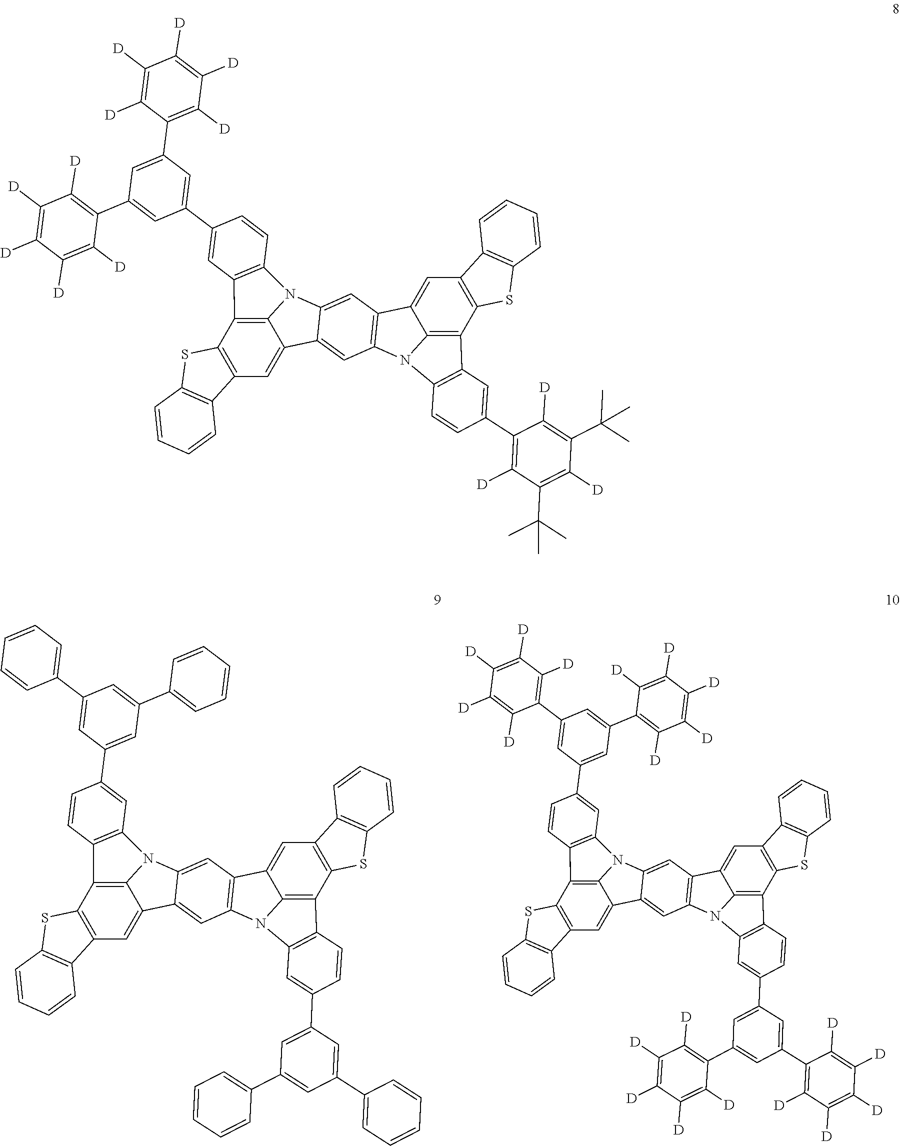

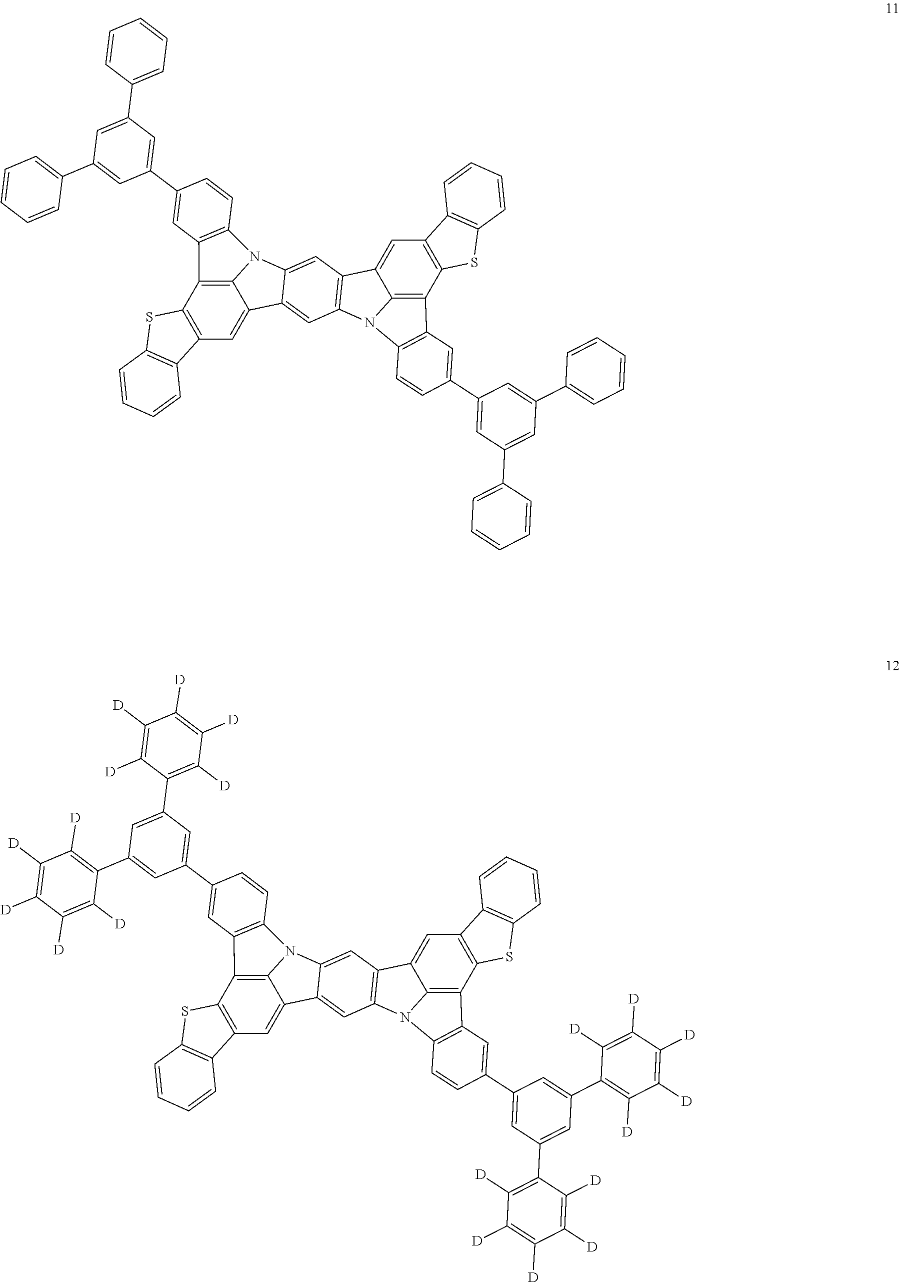 [SMILES: c1ccc(-c2cc(-c3ccccc3)cc(-c3ccc4c(c3)c3c5sc6ccccc6c5cc5c6cc7c(cc6n4c53)c3cc4c5ccccc5sc4c4c5cc(-c6cc(-c8ccccc8)cc(-c8ccccc8)c6)ccc5n7c34)c2)cc1 ; [2H]c1c([2H])c([2H])c(-c2cc(-c3ccc4c(c3)c3c5sc6ccccc6c5cc5c6cc7c(cc6n4c53)c3cc4c5ccccc5sc4c4c5cc(-c6cc(-c8c([2H])c([2H])c([2H])c([2H])c8[2H])cc(-c8c([2H])c([2H])c([2H])c([2H])c8[2H])c6)ccc5n7c34)cc(-c3c([2H])c([2H])c([2H])c([2H])c3[2H])c2)c([2H])c1[2H]]

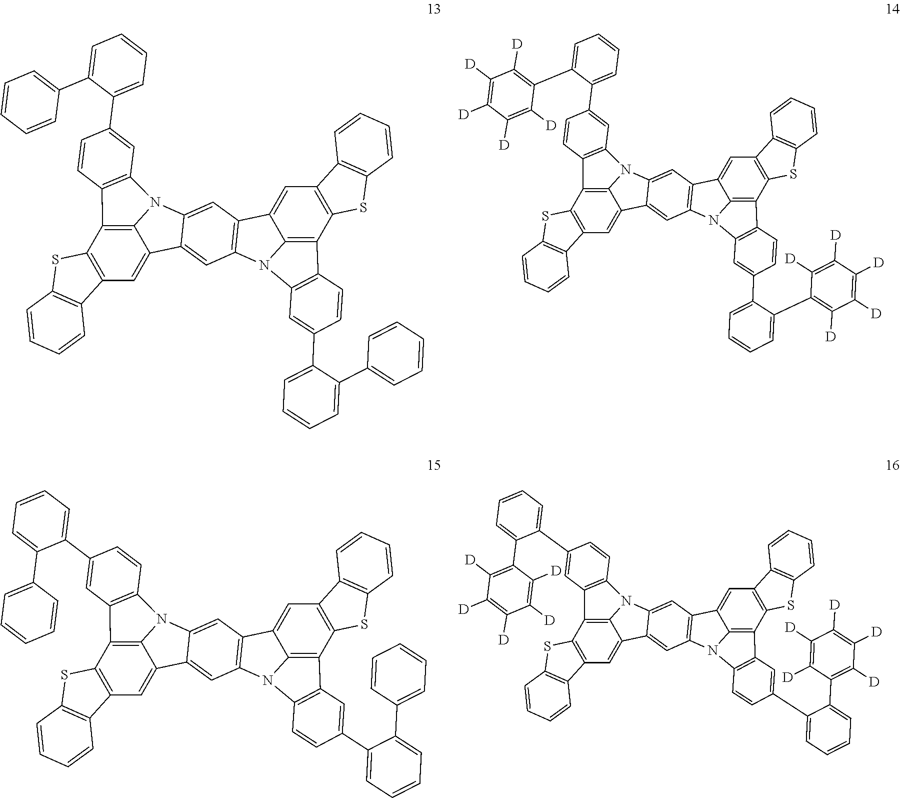

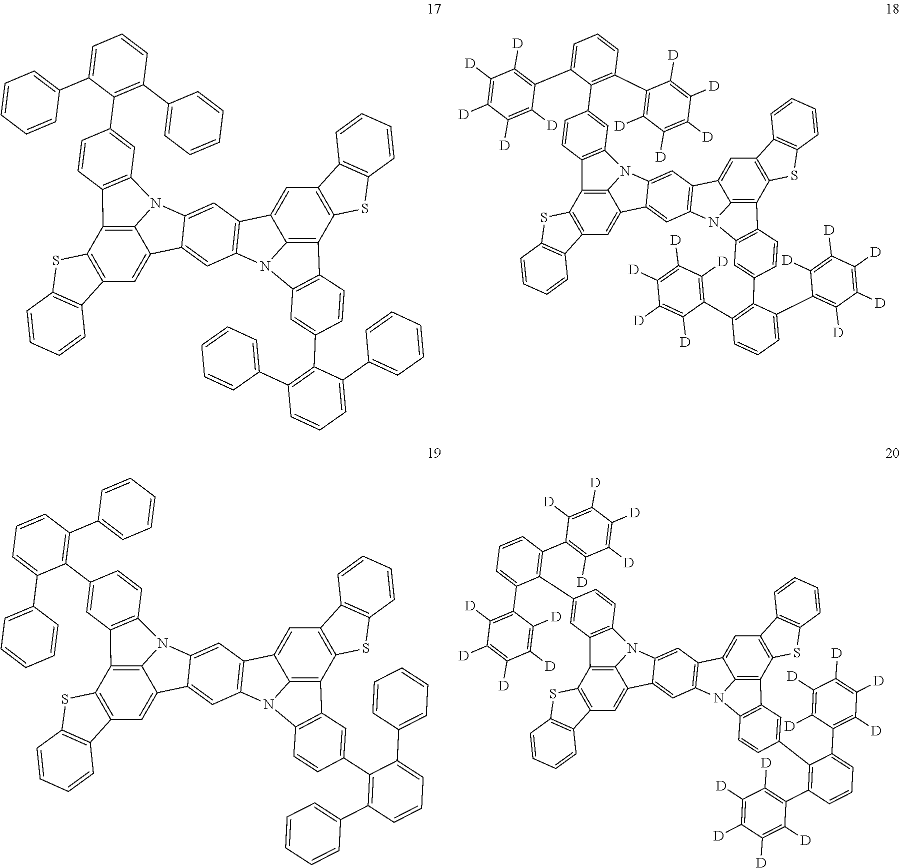

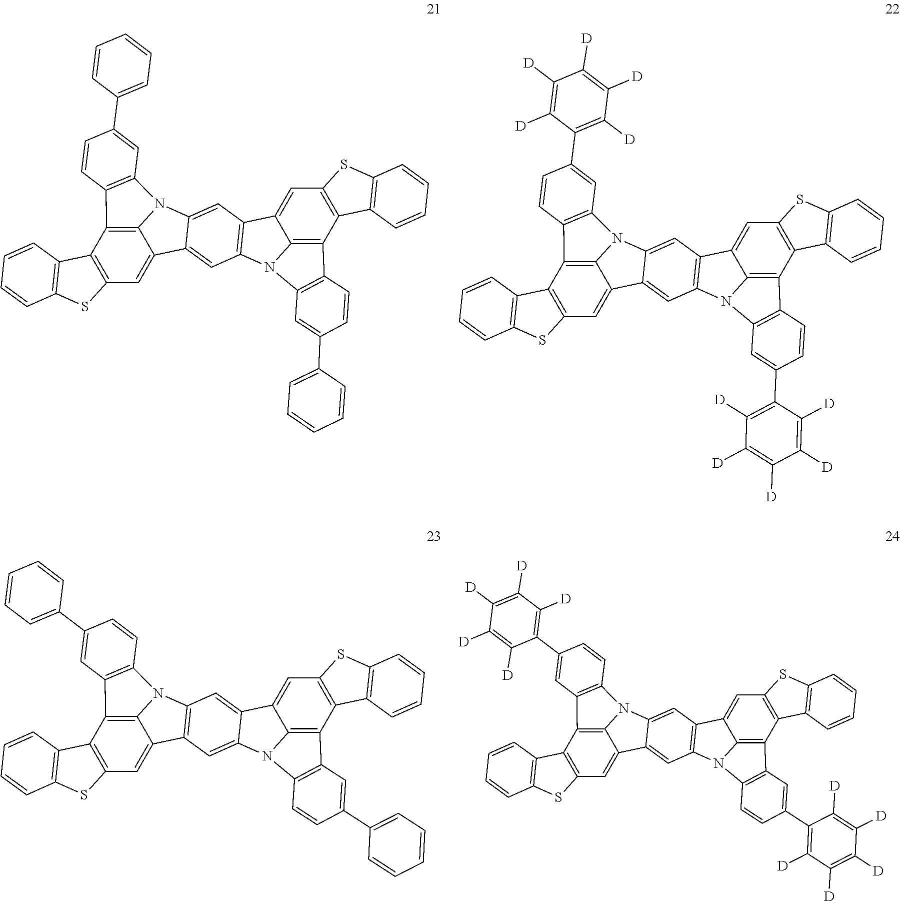

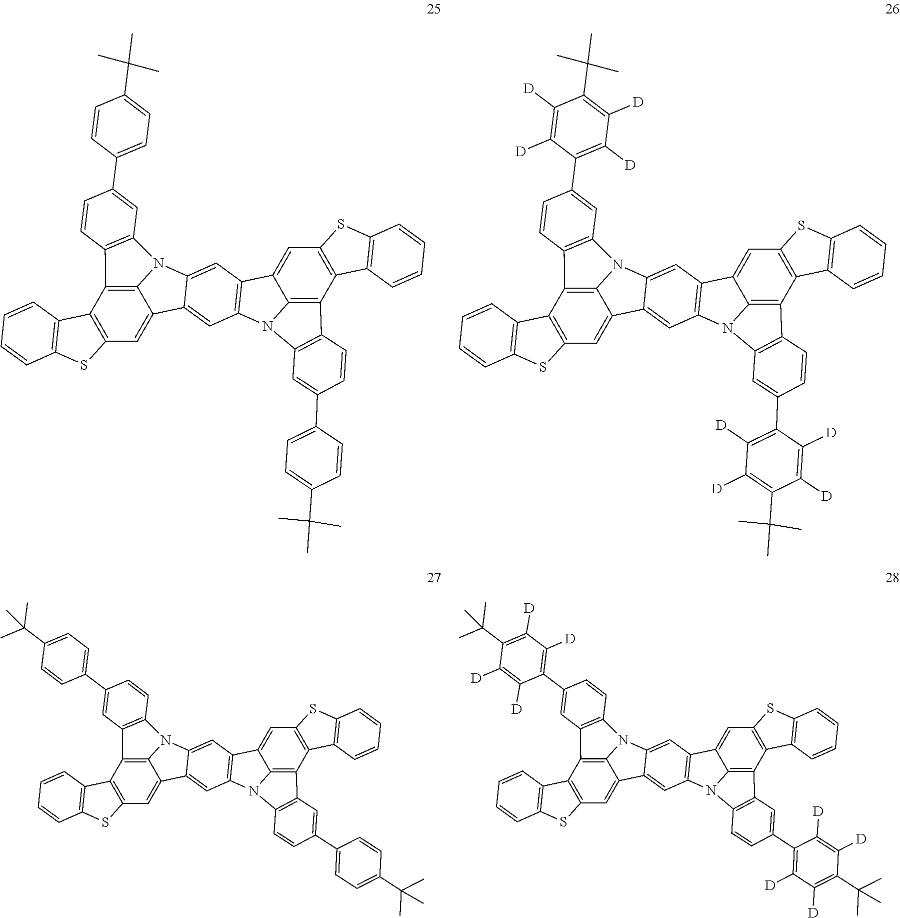

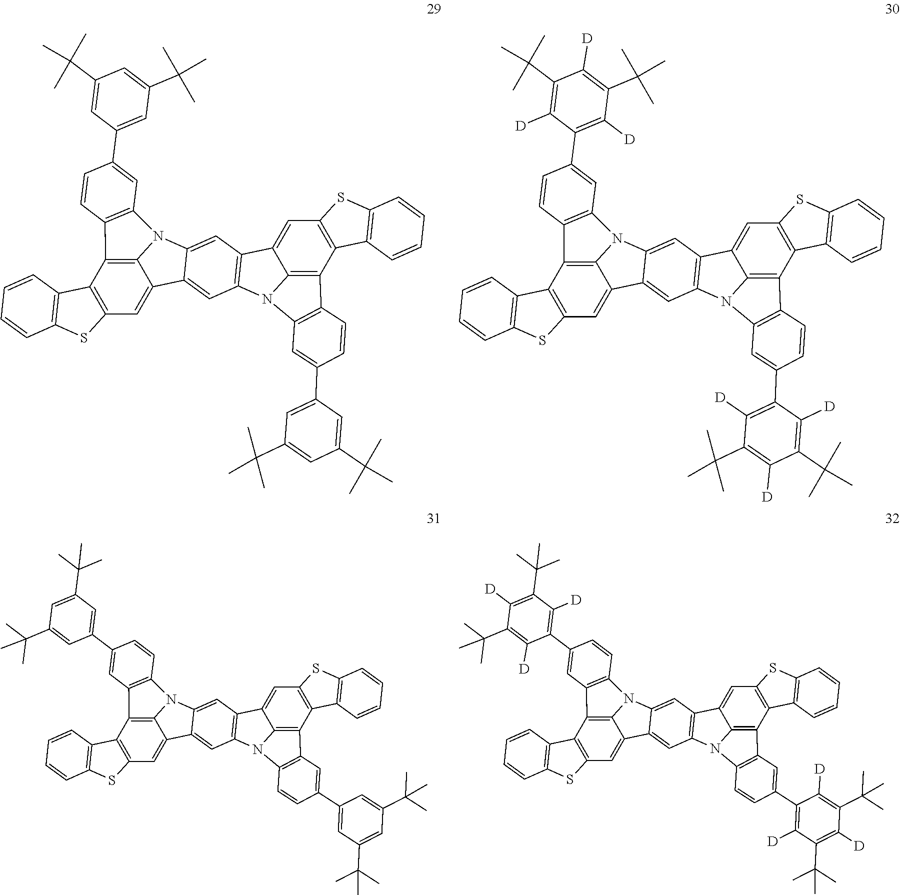

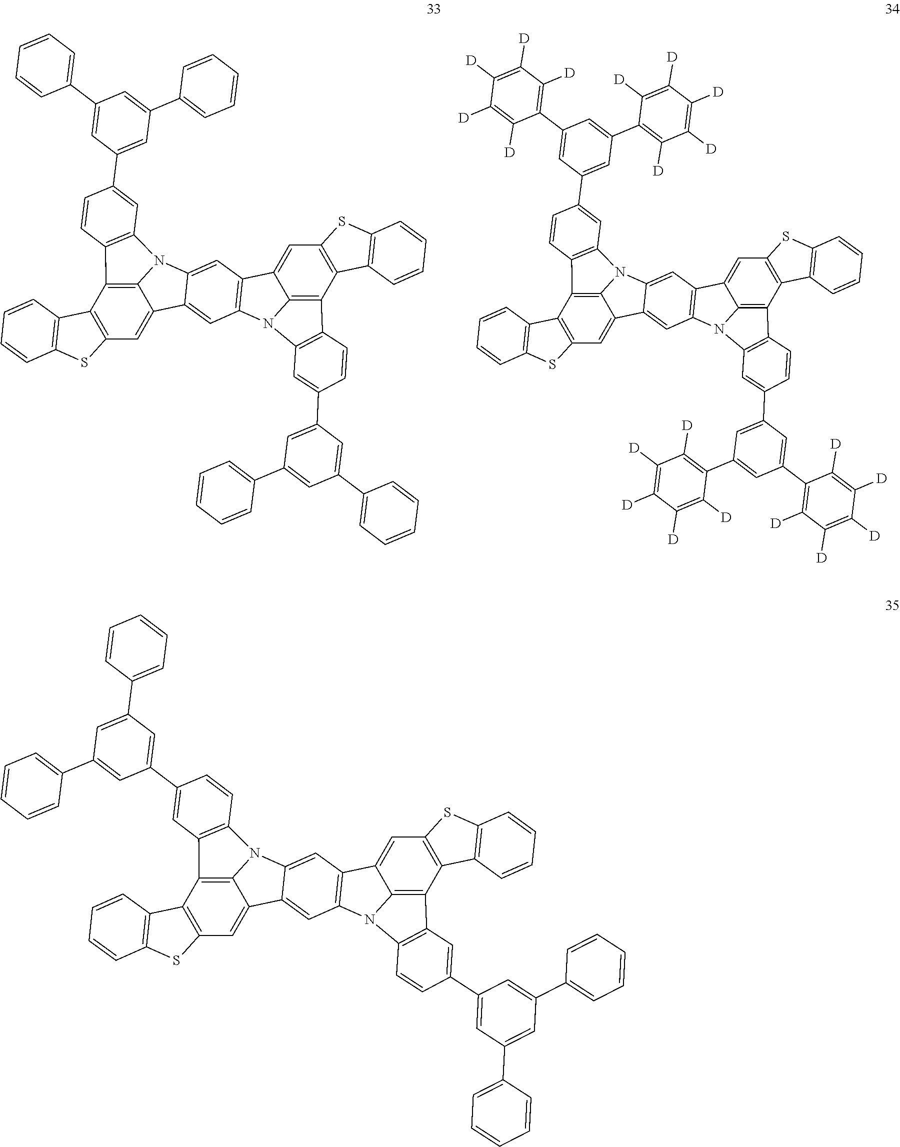

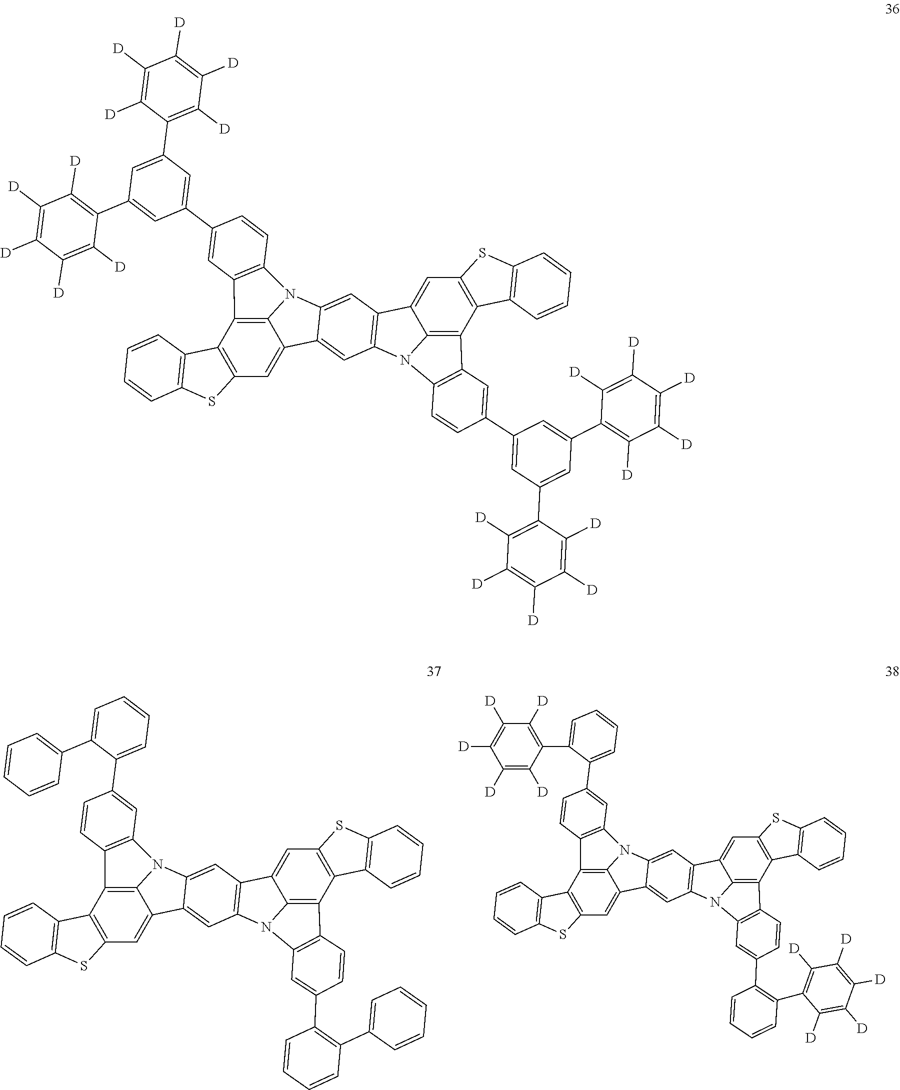

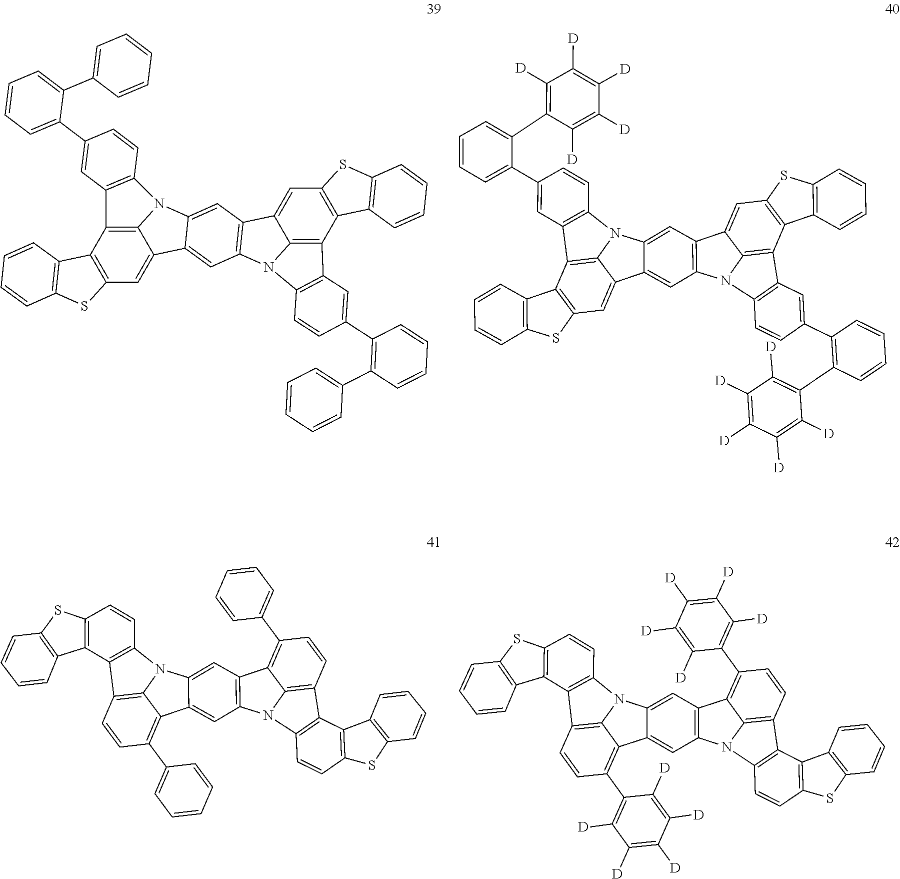

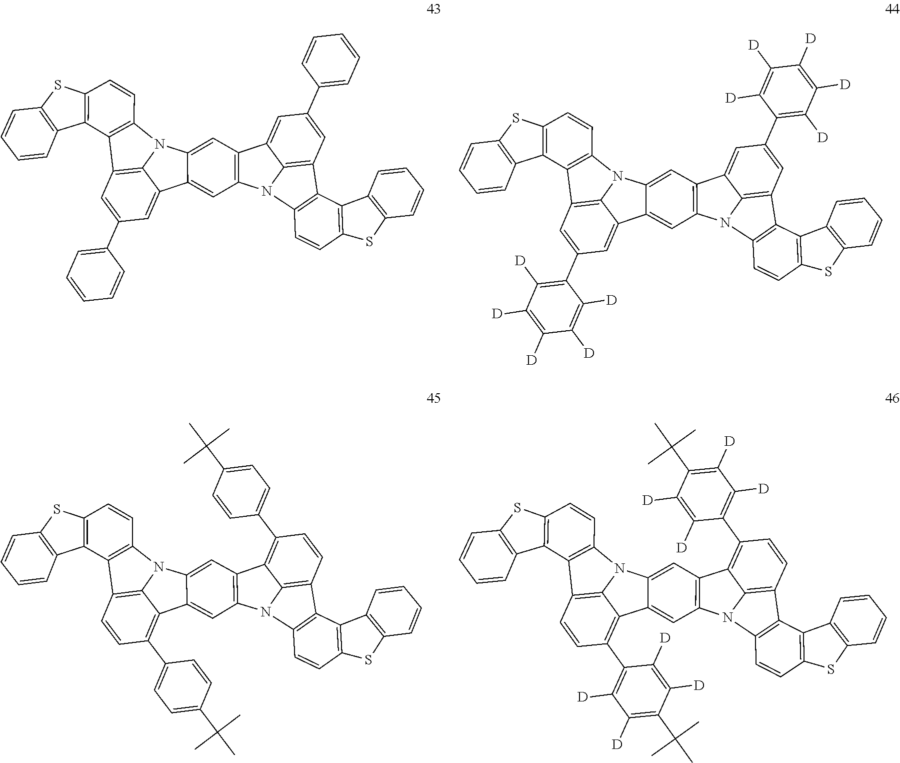

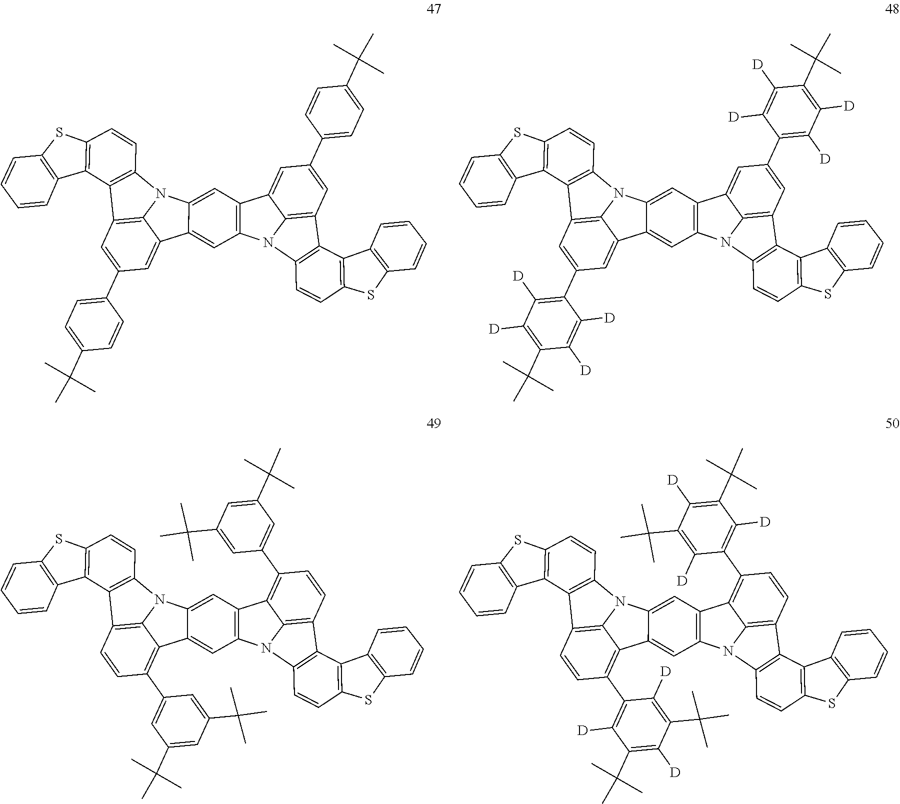

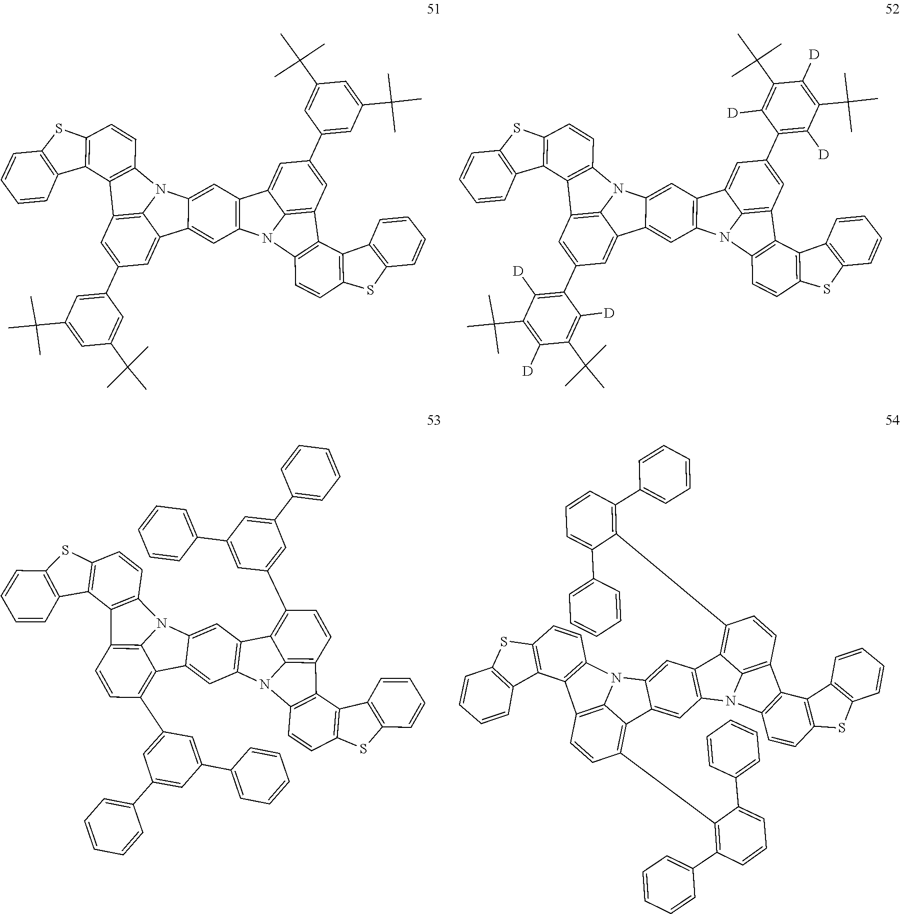

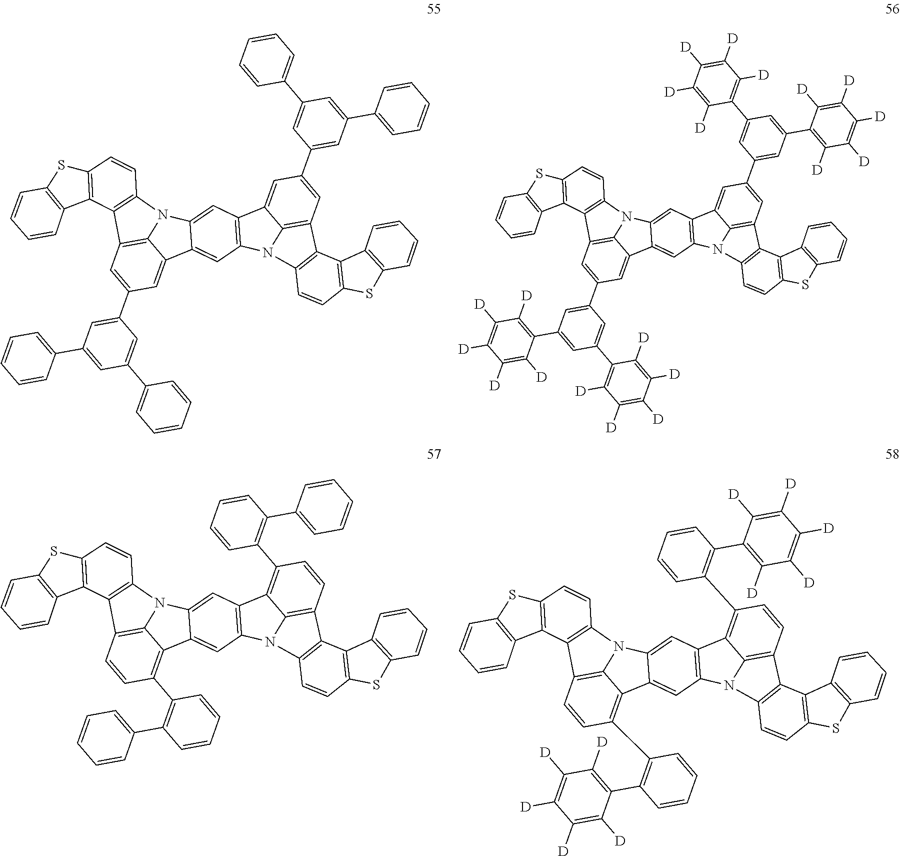

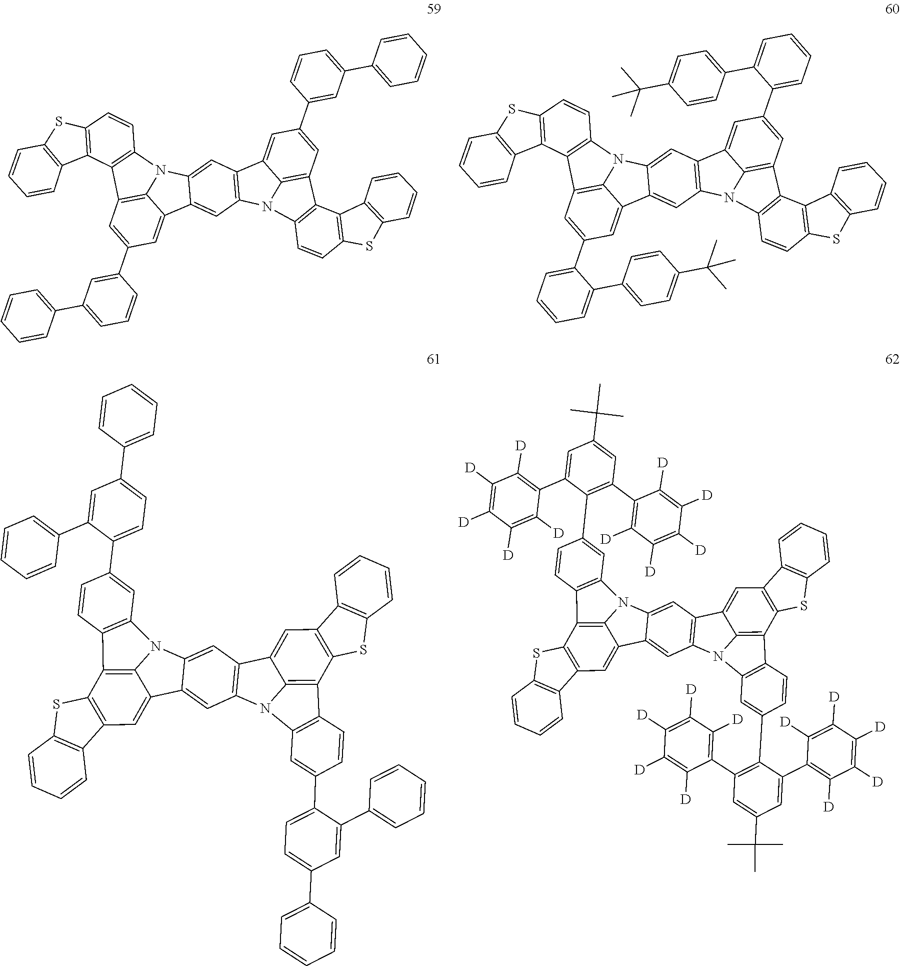

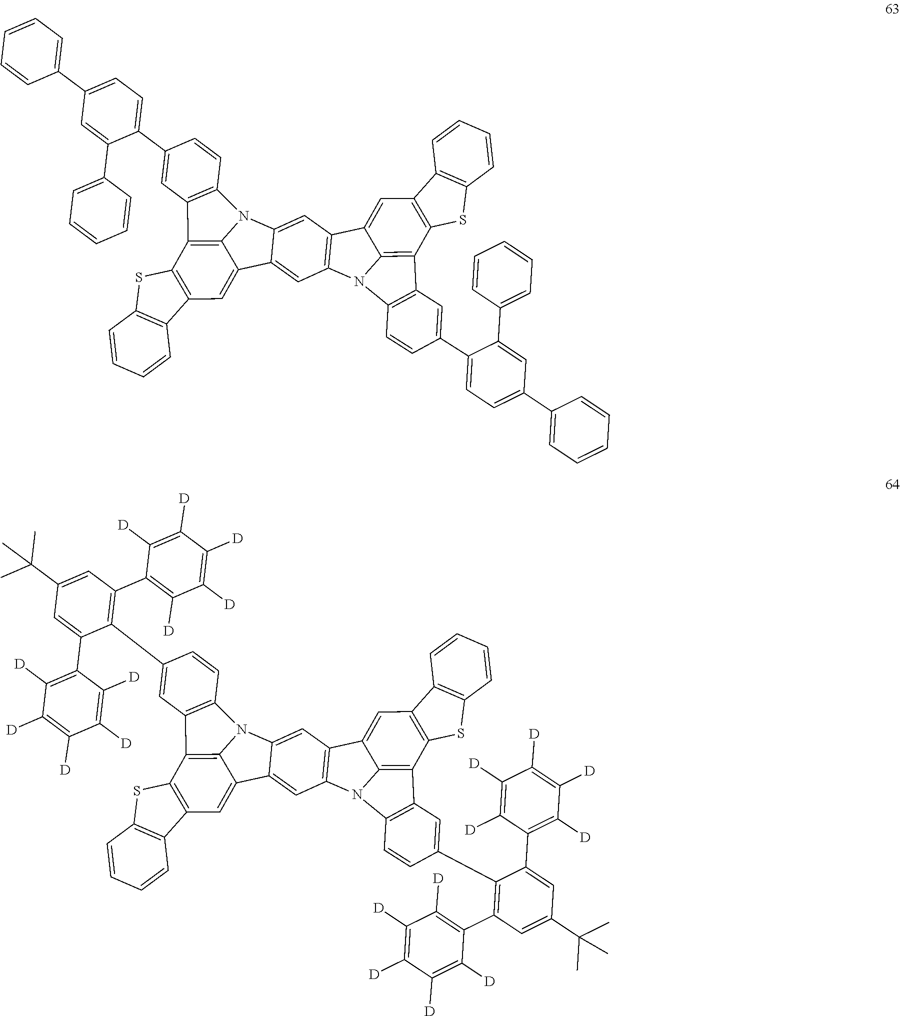 [SMILES: c1ccc(-c2ccc(-c3ccc4c(c3)c3c5sc6ccccc6c5cc5c6cc7c(cc6n4c53)c3cc4c5ccccc5sc4c4c5cc(-c6ccc(-c8ccccc8)cc6-c6ccccc6)ccc5n7c34)c(-c3ccccc3)c2)cc1 ; [2H]c1c([2H])c([2H])c(-c2cc(C(C)(C)C)cc(-c3c([2H])c([2H])c([2H])c([2H])c3[2H])c2-c2ccc3c(c2)c2c4sc5ccccc5c4cc4c5cc6c(cc5n3c42)c2cc3c4ccccc4sc3c3c4cc(-c5c(-c7c([2H])c([2H])c([2H])c([2H])c7[2H])cc(C(C)(C)C)cc5-c5c([2H])c([2H])c([2H])c([2H])c5[2H])ccc4n6c23)c([2H])c1[2H]]

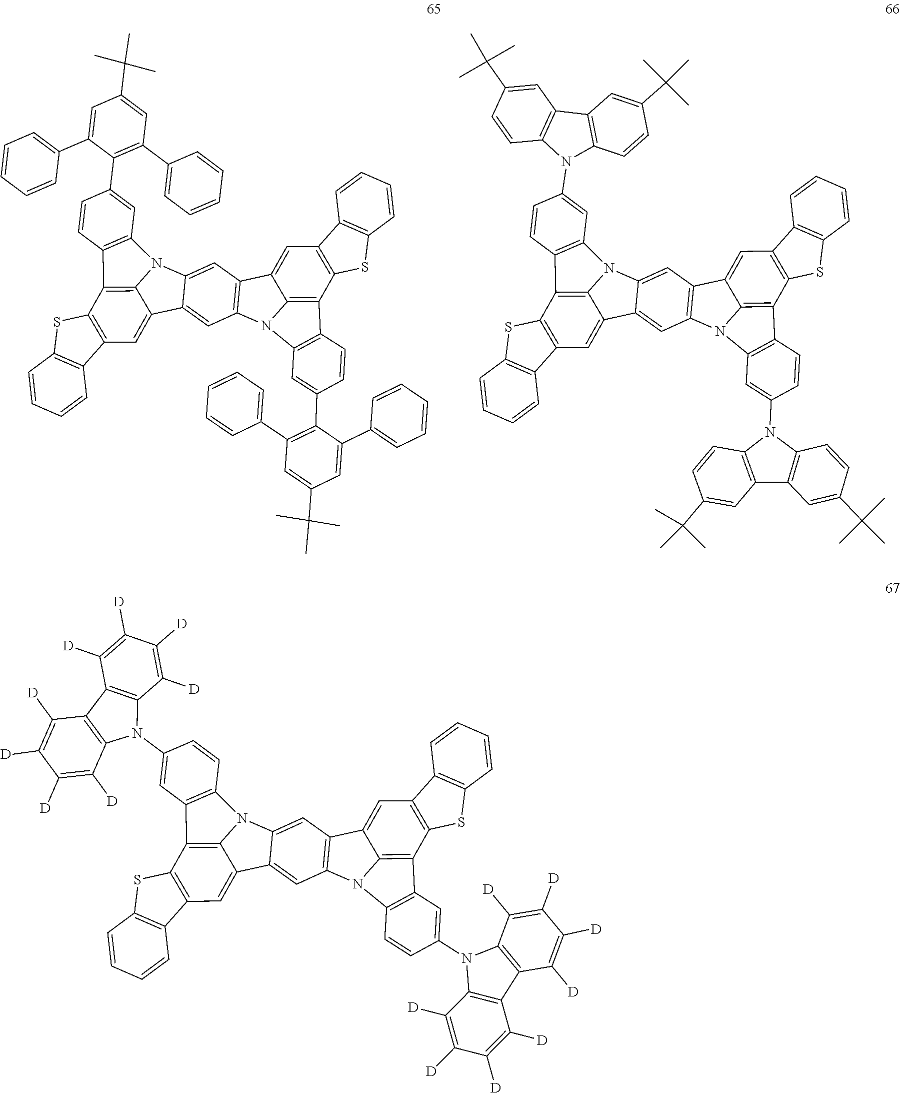

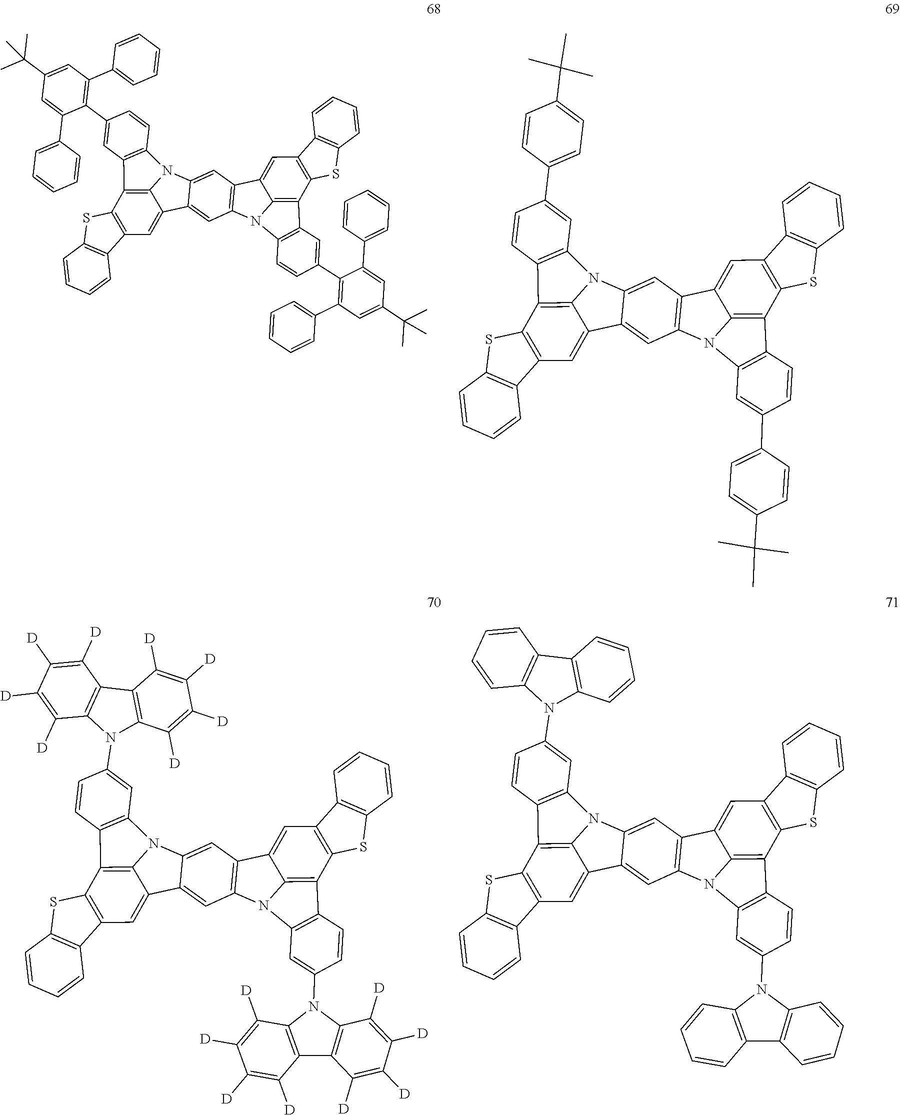

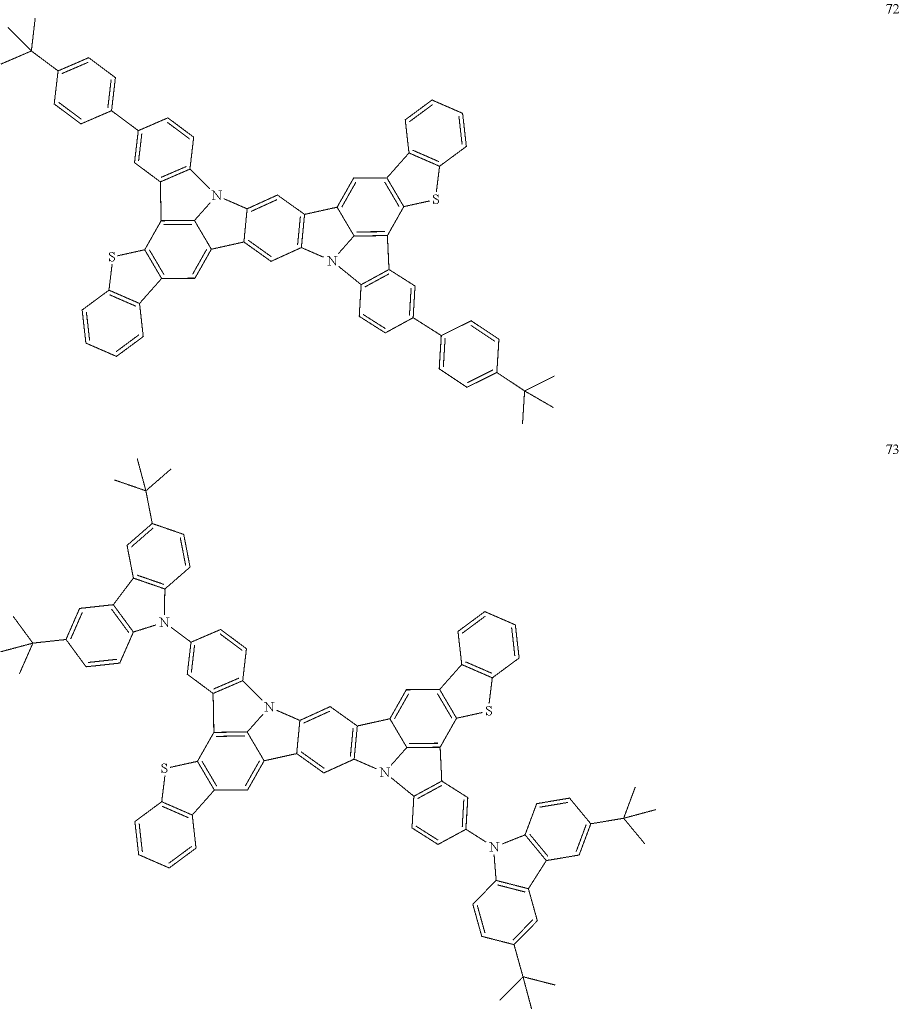

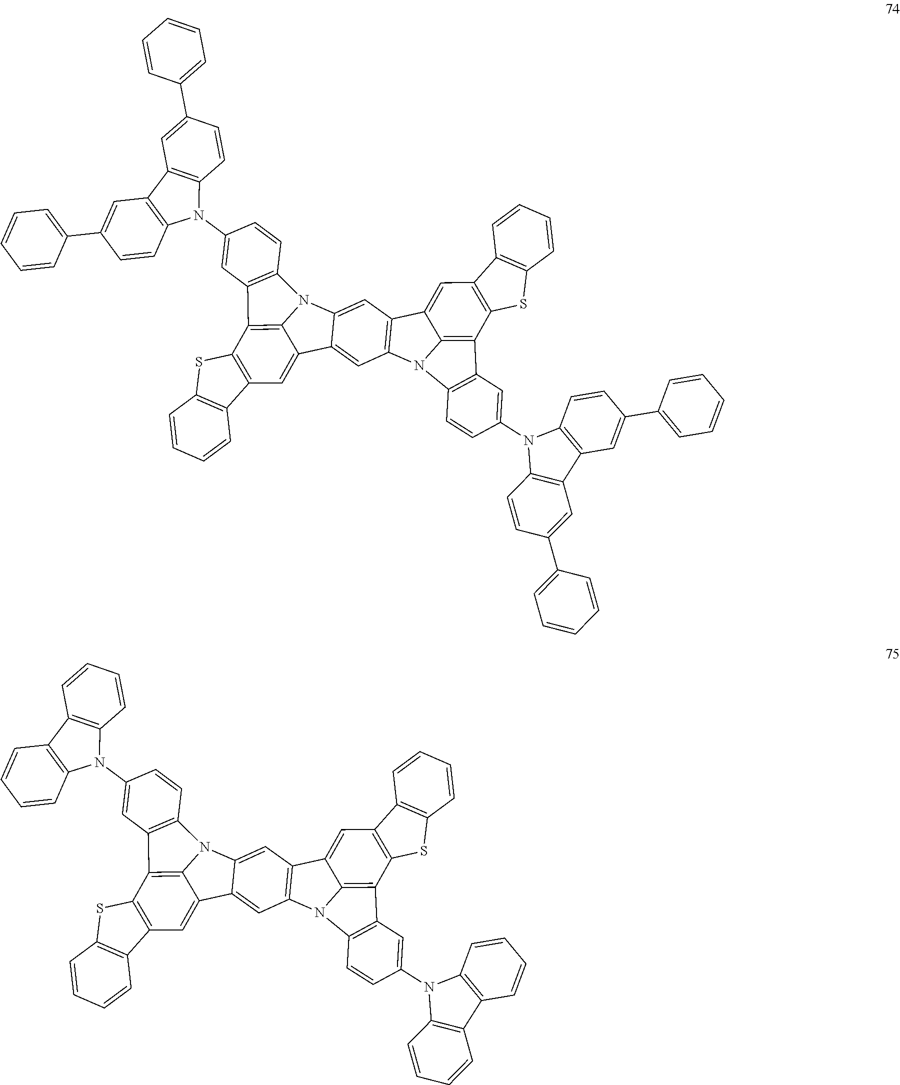 [SMILES: c1ccc(-c2ccc3c(c2)c2cc(-c4ccccc4)ccc2n3-c2ccc3c(c2)c2c4sc5ccccc5c4cc4c5cc6c(cc5n3c42)c2cc3c4ccccc4sc3c3c4cc(-n5c7ccc(-c8ccccc8)cc7c7cc(-c8ccccc8)ccc75)ccc4n6c23)cc1 ; c1ccc2c(c1)sc1c2cc2c3cc4c(cc3n3c5ccc(-n6c7ccccc7c7ccccc76)cc5c1c23)c1cc2c3ccccc3sc2c2c3cc(-n5c6ccccc6c6ccccc65)ccc3n4c12]

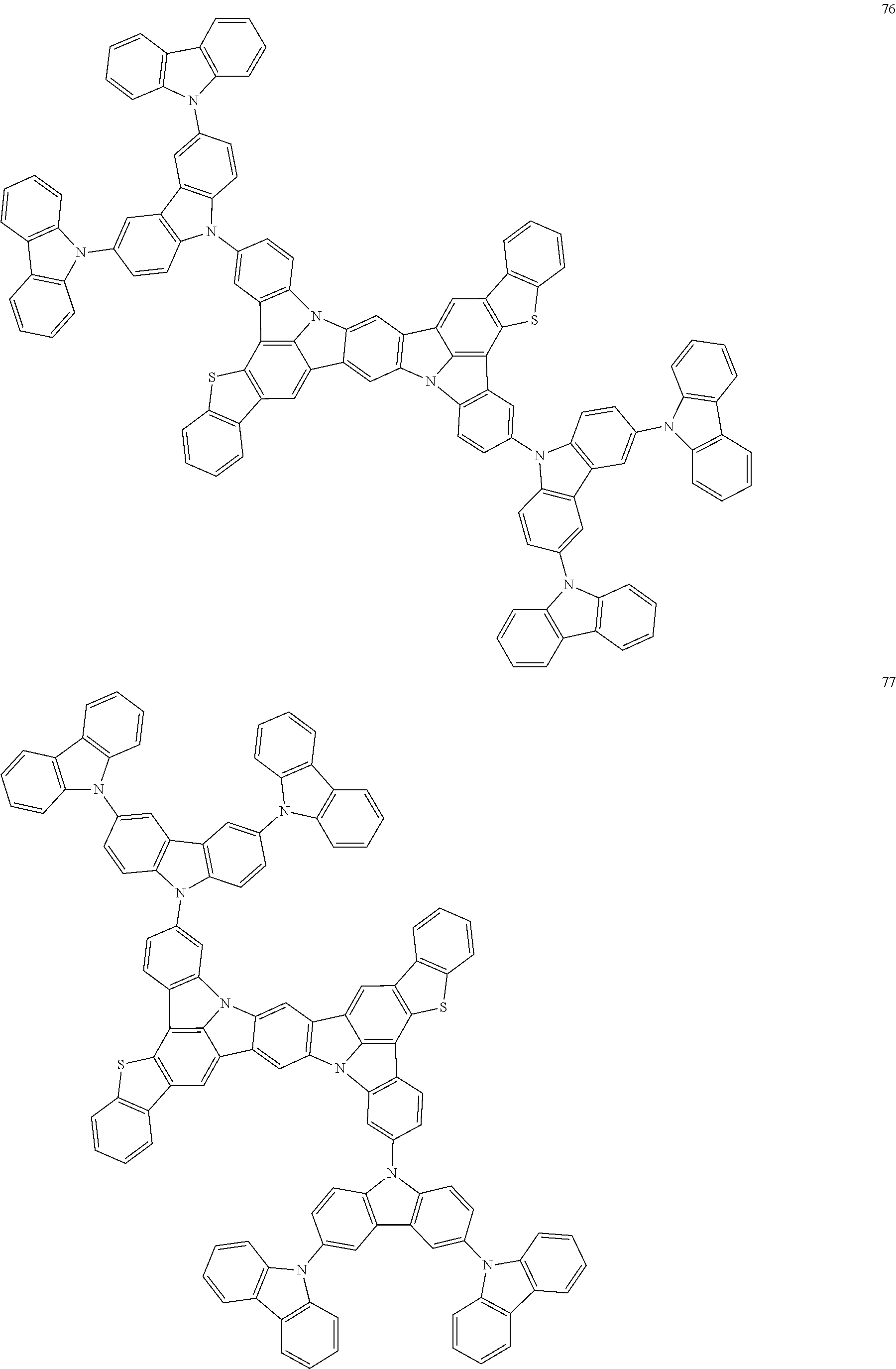 [SMILES: c1ccc2c(c1)sc1c2cc2c3cc4c(cc3n3c5ccc(-n6c7ccc(-n8c9ccccc9c9ccccc98)cc7c7cc(-n8c9ccccc9c9ccccc98)ccc76)cc5c1c23)c1cc2c3ccccc3sc2c2c3cc(-n5c6ccc(-n7c8ccccc8c8ccccc87)cc6c6cc(-n7c8ccccc8c8ccccc87)ccc65)ccc3n4c12 ; c1ccc2c(c1)sc1c2cc2c3cc4c(cc3n3c5cc(-n6c7ccc(-n8c9ccccc9c9ccccc98)cc7c7cc(-n8c9ccccc9c9ccccc98)ccc76)ccc5c1c23)c1cc2c3ccccc3sc2c2c3ccc(-n5c6ccc(-n7c8ccccc8c8ccccc87)cc6c6cc(-n7c8ccccc8c8ccccc87)ccc65)cc3n4c12]

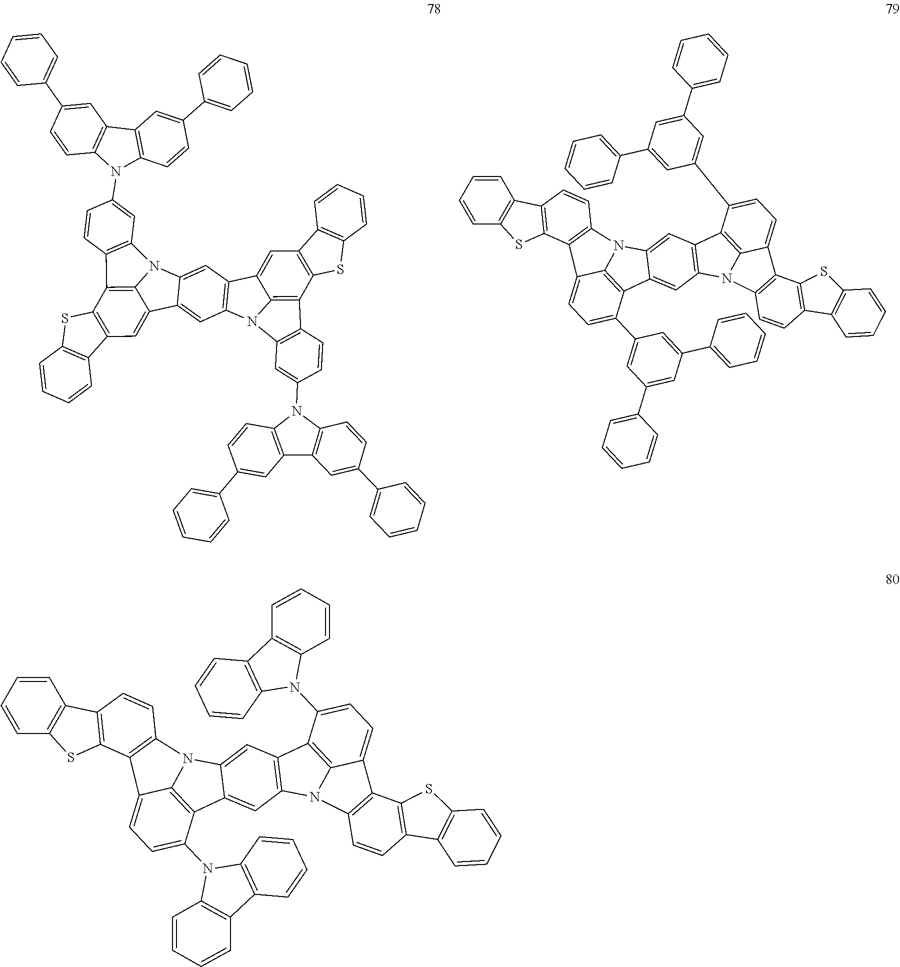 [SMILES: c1ccc(-c2ccc3c(c2)c2cc(-c4ccccc4)ccc2n3-c2ccc3c4c5sc6ccccc6c5cc5c6cc7c(cc6n(c3c2)c54)c2cc3c4ccccc4sc3c3c4ccc(-n5c6ccc(-c8ccccc8)cc6c6cc(-c8ccccc8)ccc65)cc4n7c23)cc1 ; c1ccc(-c2cc(-c3ccccc3)cc(-c3ccc4c5c6sc7ccccc7c6ccc5n5c6cc7c8c(-c9cc(-c%10ccccc%10)cc(-c%10ccccc%10)c9)ccc9c%10c%11sc%12ccccc%12c%11ccc%10n(c7cc6c3c45)c98)c2)cc1 ; c1ccc2c(c1)sc1c2ccc2c1c1ccc(-n3c4ccccc4c4ccccc43)c3c4cc5c(cc4n2c13)c1c(-n2c3ccccc3c3ccccc32)ccc2c3c4sc6ccccc6c4ccc3n5c21]

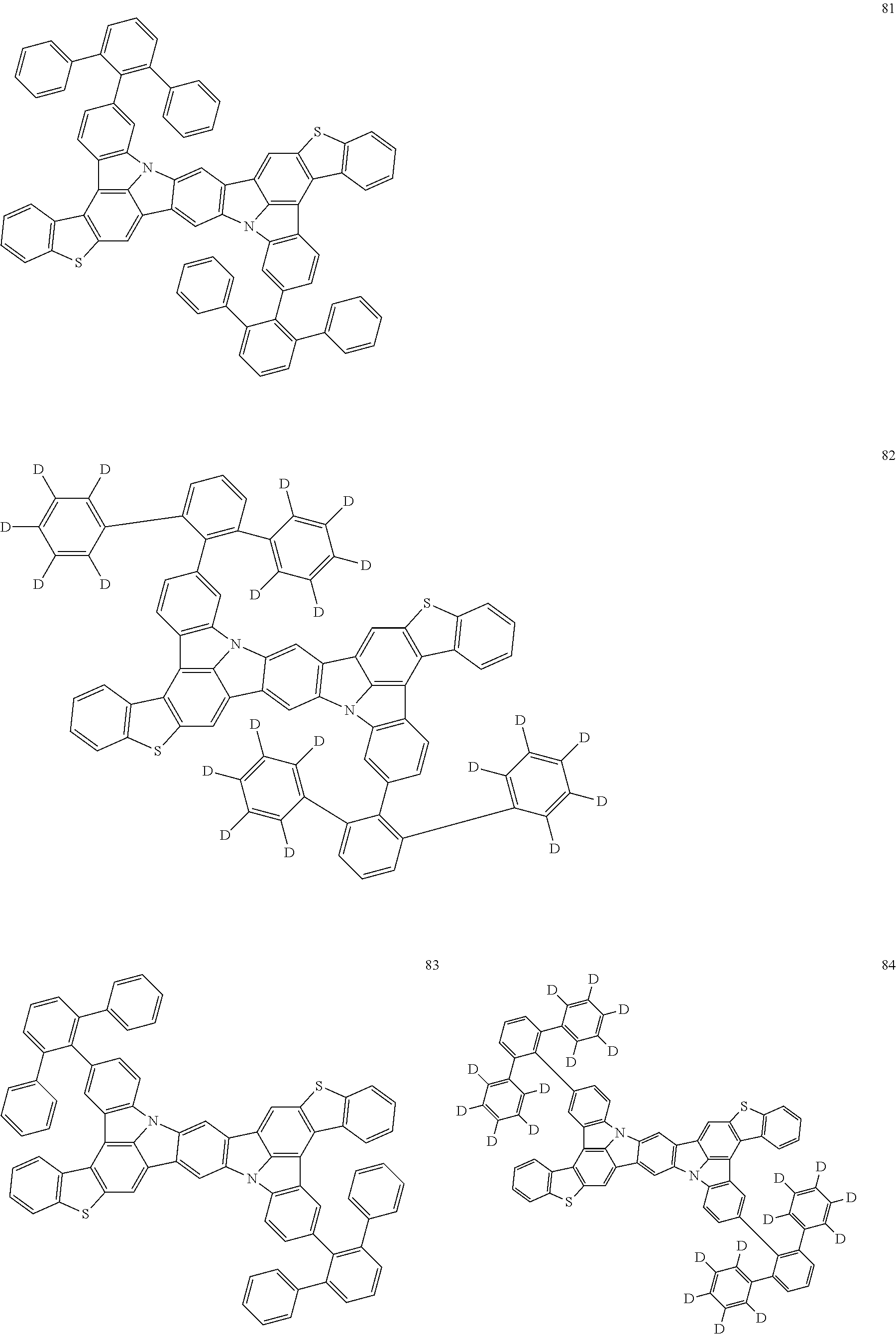

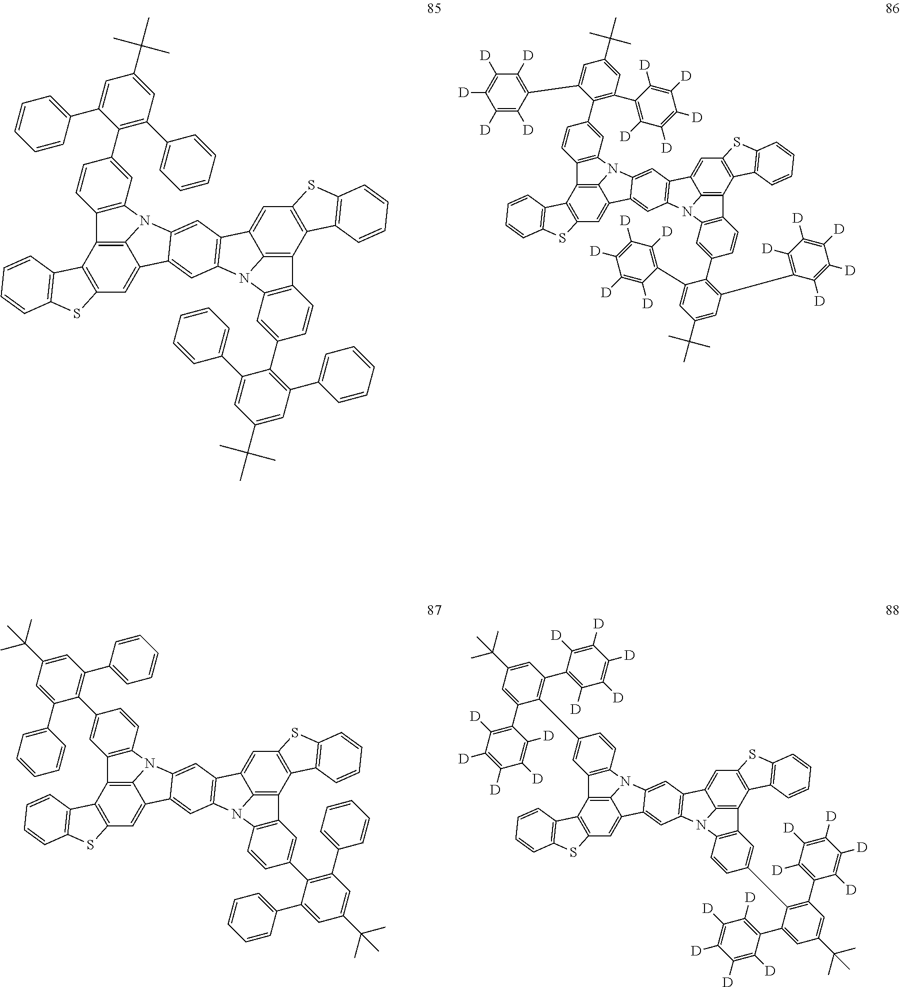

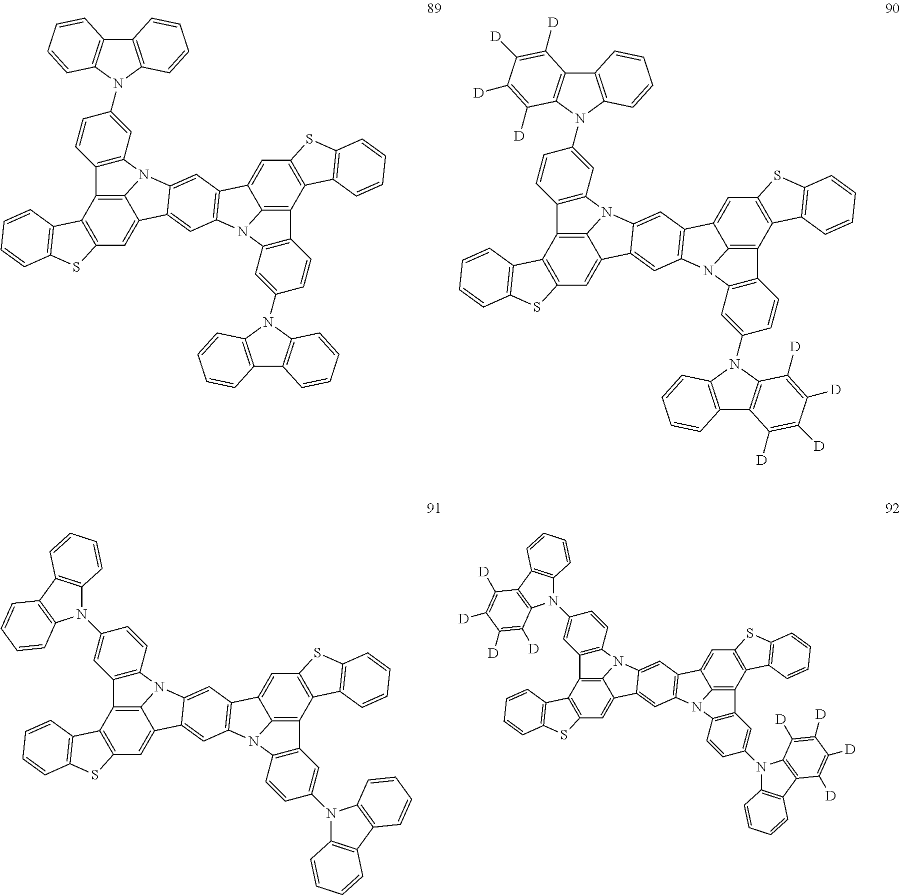

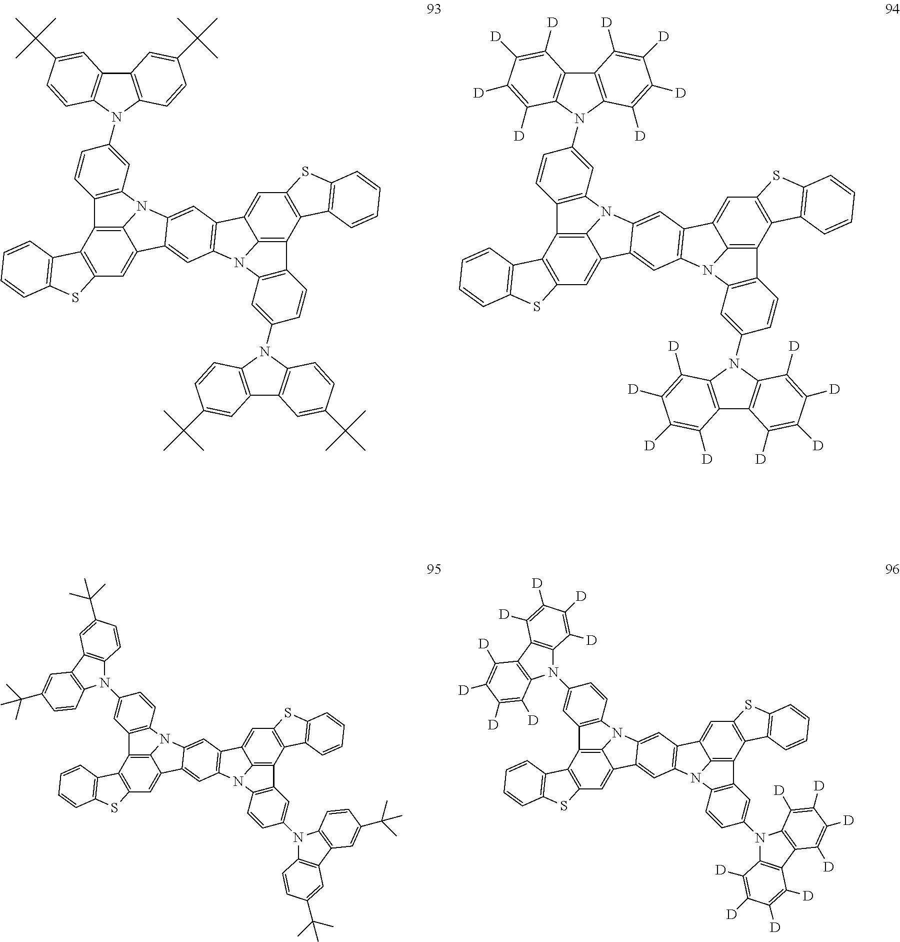

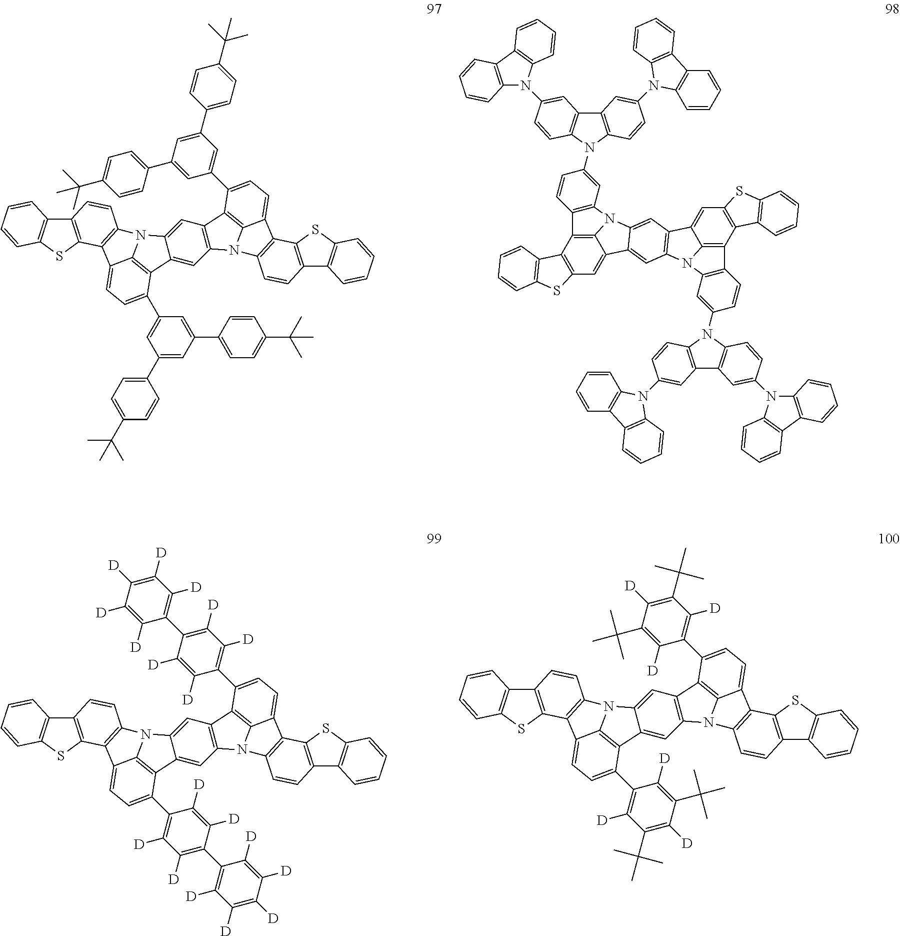

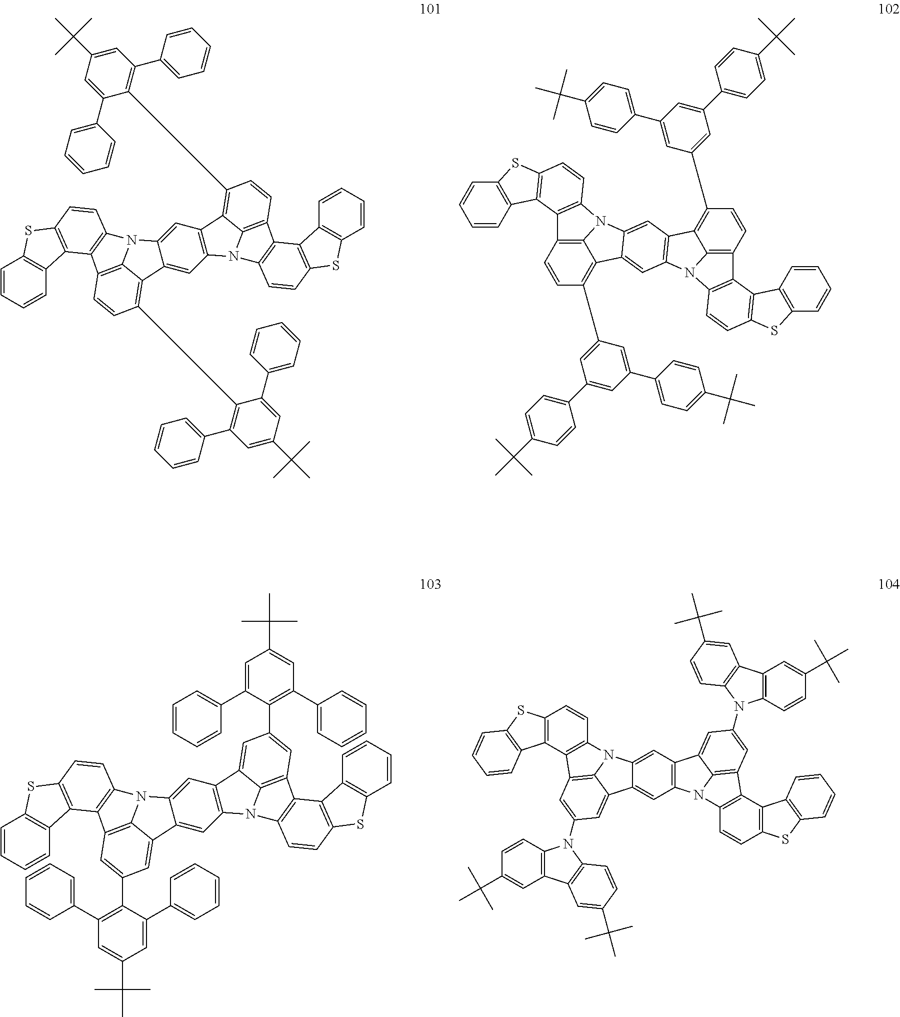

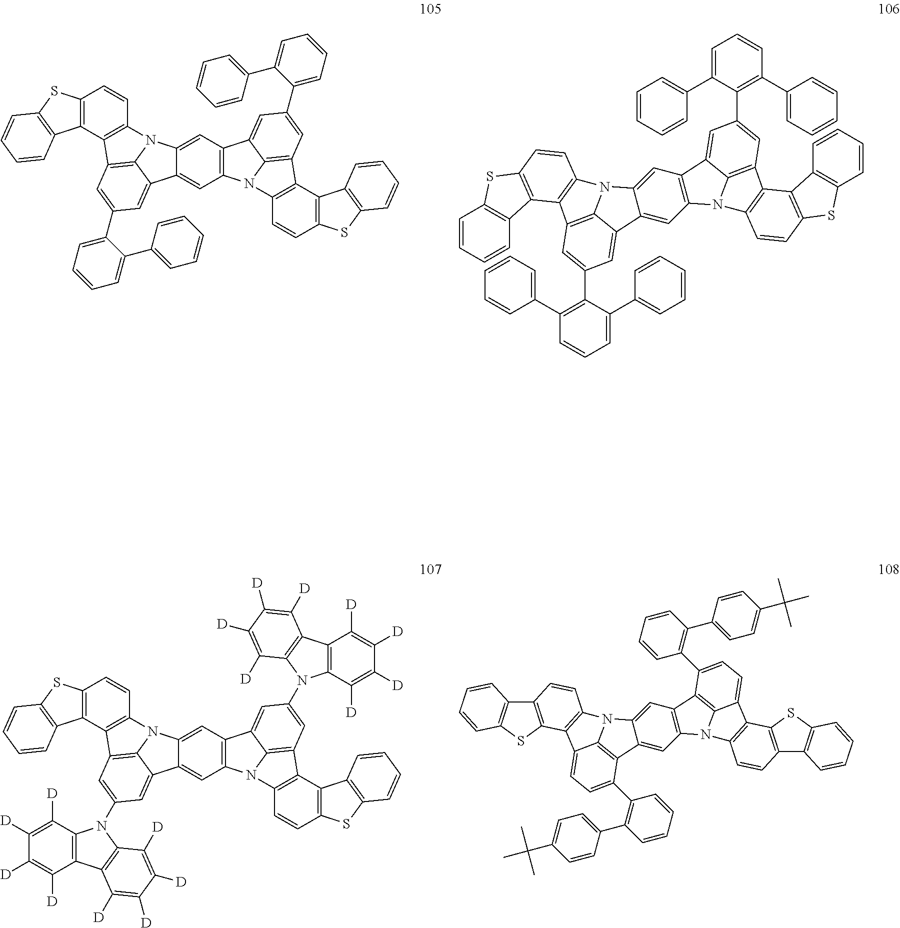

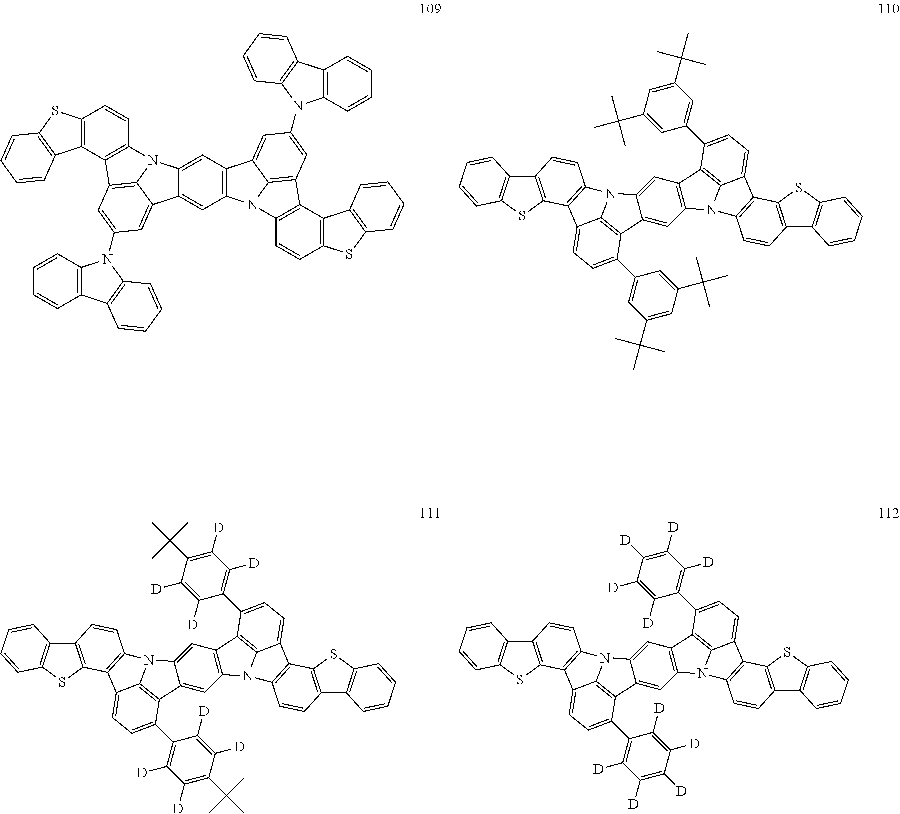

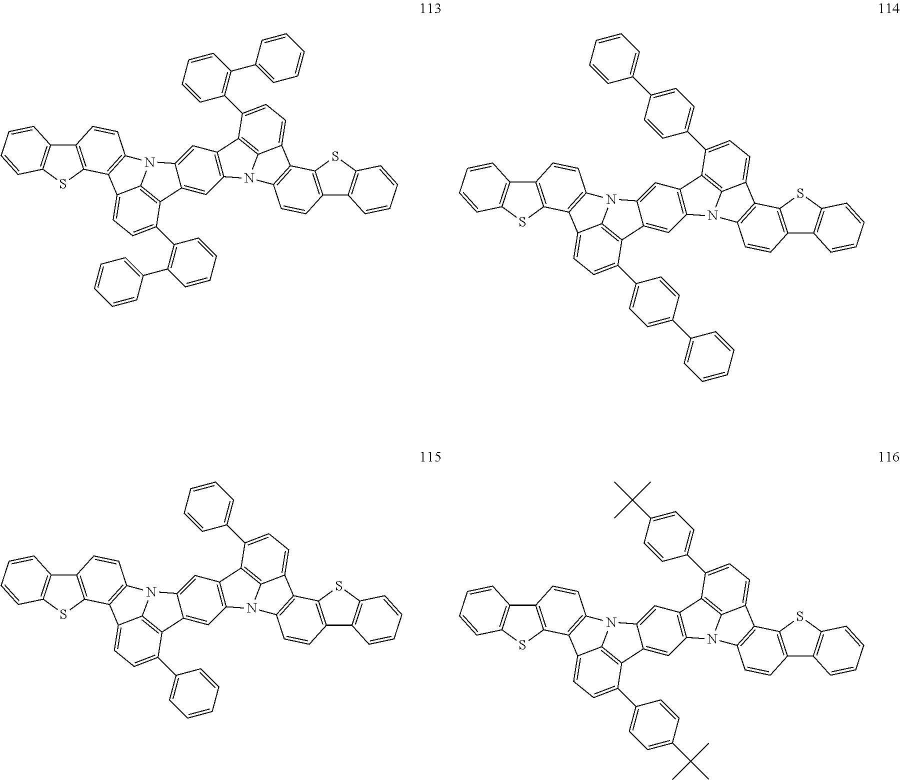 [SMILES: c1ccc(-c2ccccc2-c2ccc3c4c5sc6ccccc6c5ccc4n4c5cc6c7c(-c8ccccc8-c8ccccc8)ccc8c9c%10sc%11ccccc%11c%10ccc9n(c6cc5c2c34)c87)cc1 ; c1ccc(-c2ccc(-c3ccc4c5c6sc7ccccc7c6ccc5n5c6cc7c8c(-c9ccc(-c%10ccccc%10)cc9)ccc9c%10c%11sc%12ccccc%12c%11ccc%10n(c7cc6c3c45)c98)cc2)cc1 ; c1ccc(-c2ccc3c4c5sc6ccccc6c5ccc4n4c5cc6c7c(-c8ccccc8)ccc8c9c%10sc%11ccccc%11c%10ccc9n(c6cc5c2c34)c87)cc1 ; CC(C)(C)c1ccc(-c2ccc3c4c5sc6ccccc6c5ccc4n4c5cc6c7c(-c8ccc(C(C)(C)C)cc8)ccc8c9c%10sc%11ccccc%11c%10ccc9n(c6cc5c2c34)c87)cc1]

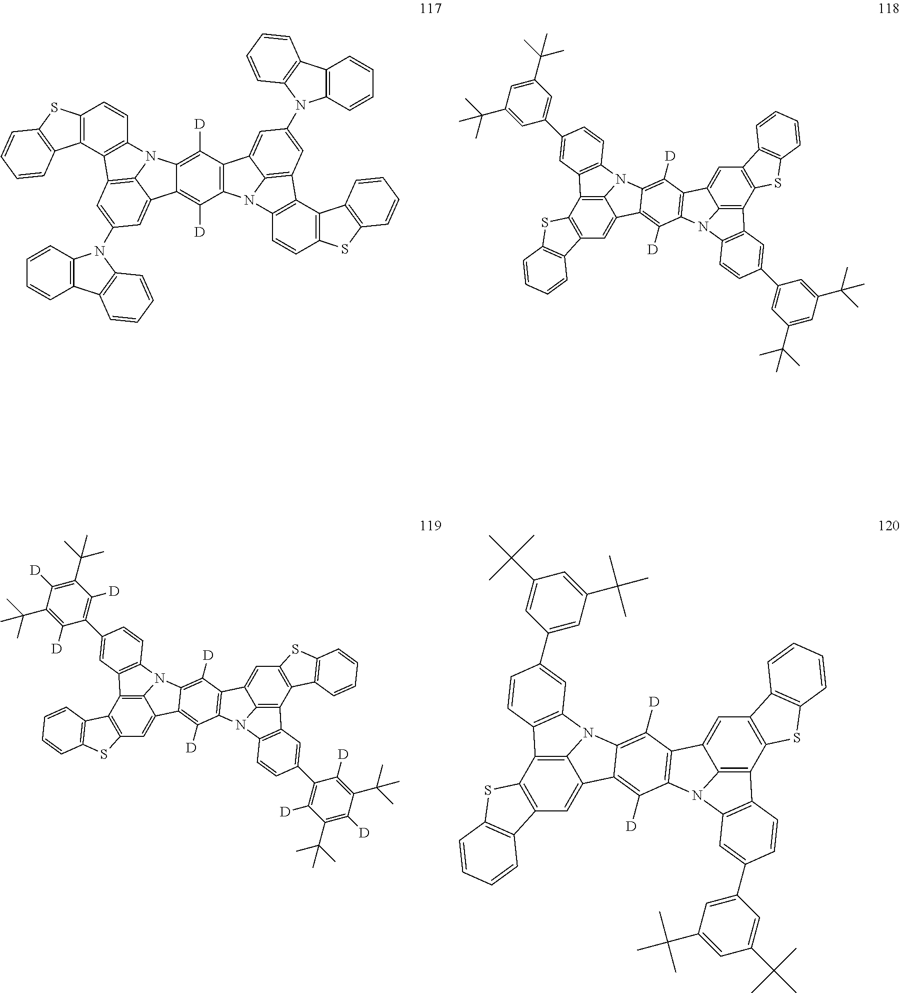

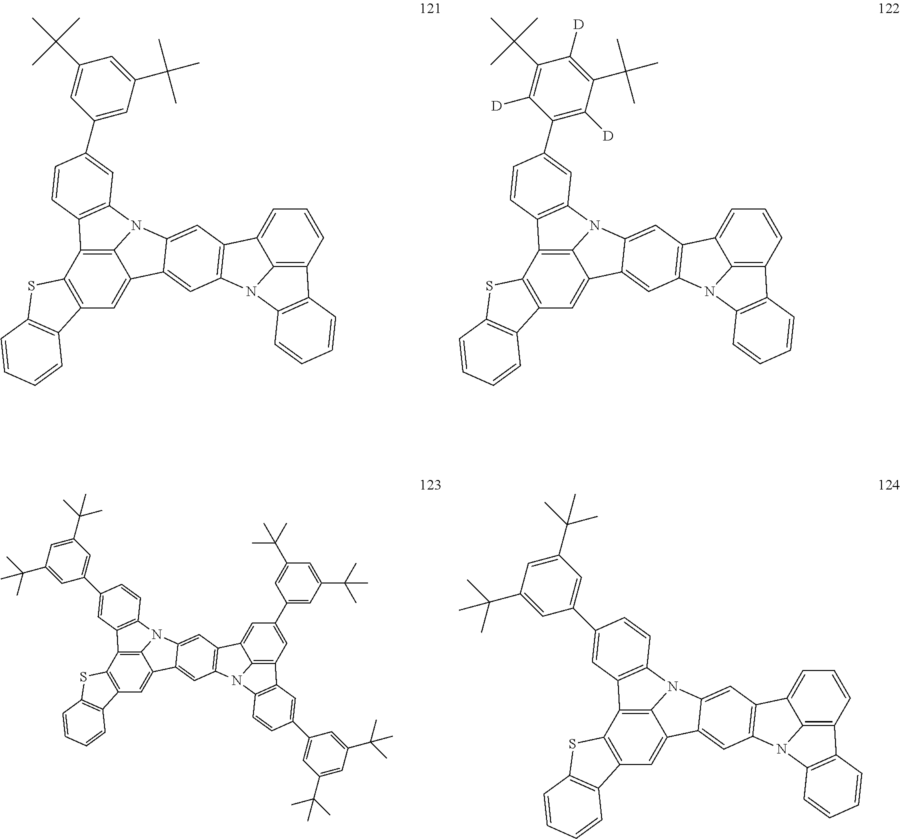 [SMILES: CC(C)(C)c1cc(-c2ccc3c4c5sc6ccccc6c5cc5c6cc7c(cc6n(c3c2)c54)c2cccc3c4ccccc4n7c32)cc(C(C)(C)C)c1 ; [2H]c1c(-c2ccc3c4c5sc6ccccc6c5cc5c6cc7c(cc6n(c3c2)c54)c2cccc3c4ccccc4n7c32)c([2H])c(C(C)(C)C)c([2H])c1C(C)(C)C ; CC(C)(C)c1cc(-c2ccc3c(c2)c2cc(-c4cc(C(C)(C)C)cc(C(C)(C)C)c4)cc4c5cc6c(cc5n3c24)c2cc3c4ccccc4sc3c3c4cc(-c5cc(C(C)(C)C)cc(C(C)(C)C)c5)ccc4n6c23)cc(C(C)(C)C)c1 ; CC(C)(C)c1cc(-c2ccc3c(c2)c2c4sc5ccccc5c4cc4c5cc6c(cc5n3c42)c2cccc3c4ccccc4n6c32)cc(C(C)(C)C)c1]

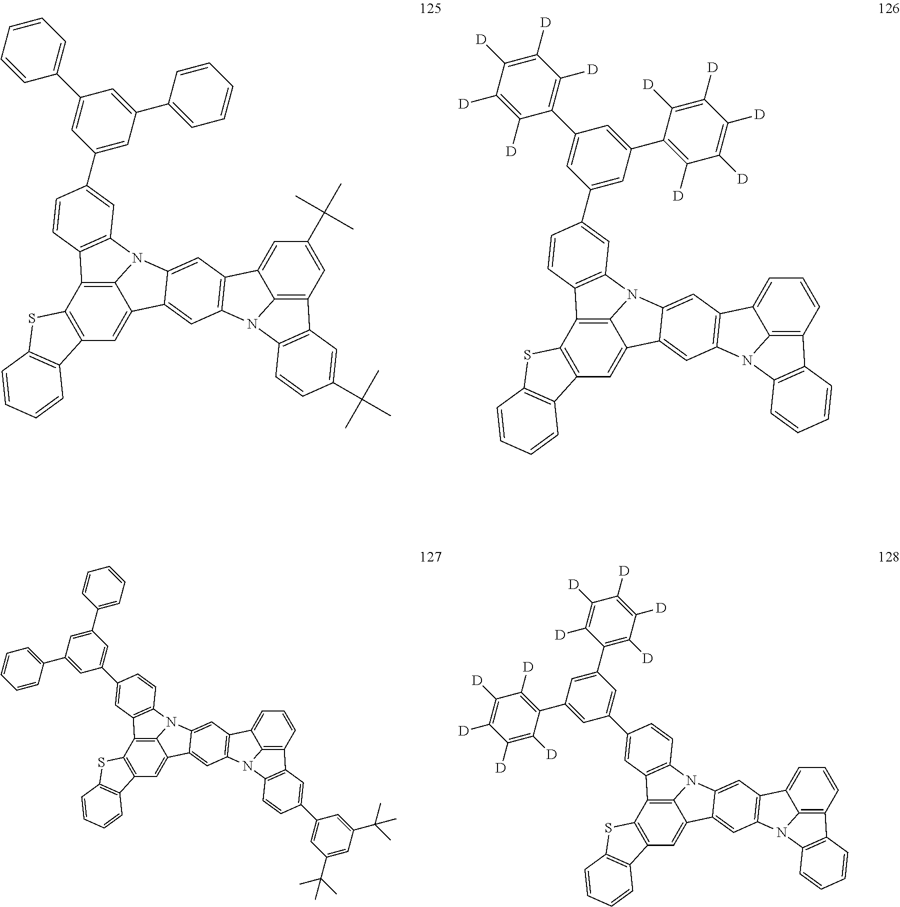

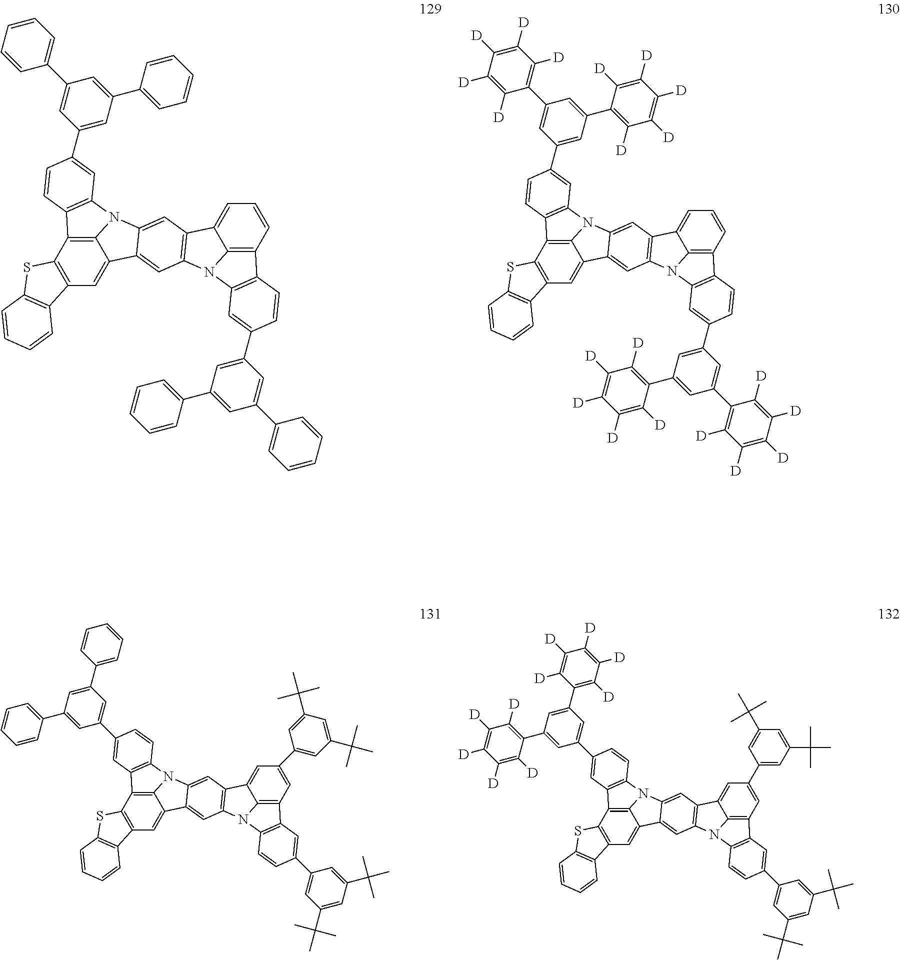

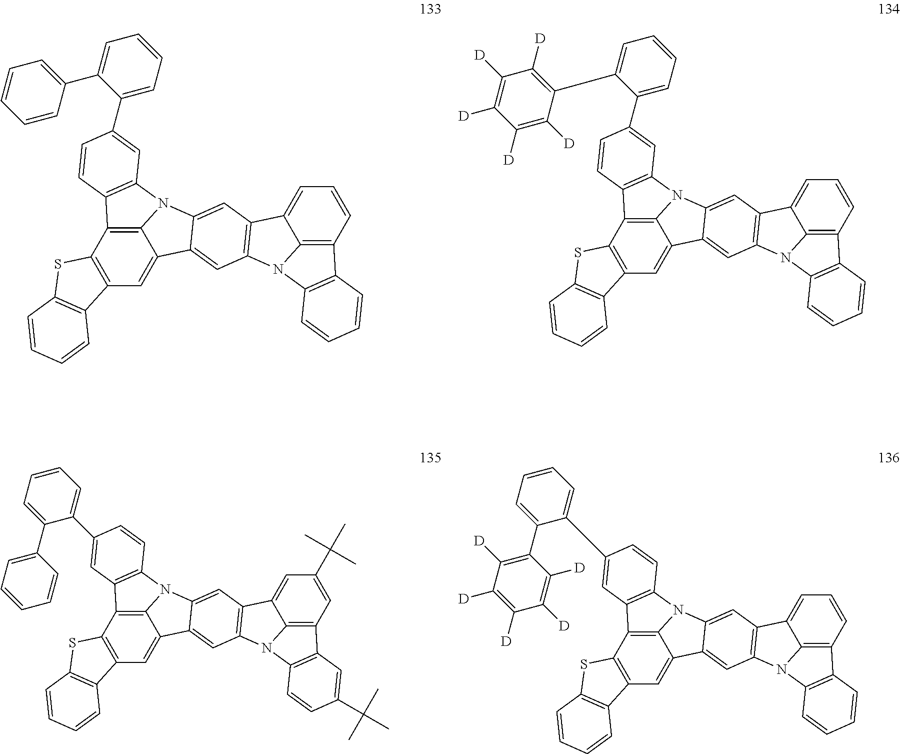 [SMILES: c1ccc(-c2ccccc2-c2ccc3c4c5sc6ccccc6c5cc5c6cc7c(cc6n(c3c2)c54)c2cccc3c4ccccc4n7c32)cc1 ; [2H]c1c([2H])c([2H])c(-c2ccccc2-c2ccc3c4c5sc6ccccc6c5cc5c6cc7c(cc6n(c3c2)c54)c2cccc3c4ccccc4n7c32)c([2H])c1[2H] ; CC(C)(C)c1ccc2c(c1)c1cc(C(C)(C)C)cc3c4cc5c(cc4n2c13)c1cc2c3ccccc3sc2c2c3cc(-c4ccccc4-c4ccccc4)ccc3n5c12 ; [2H]c1c([2H])c([2H])c(-c2ccccc2-c2ccc3c(c2)c2c4sc5ccccc5c4cc4c5cc6c(cc5n3c42)c2cccc3c4ccccc4n6c32)c([2H])c1[2H]]

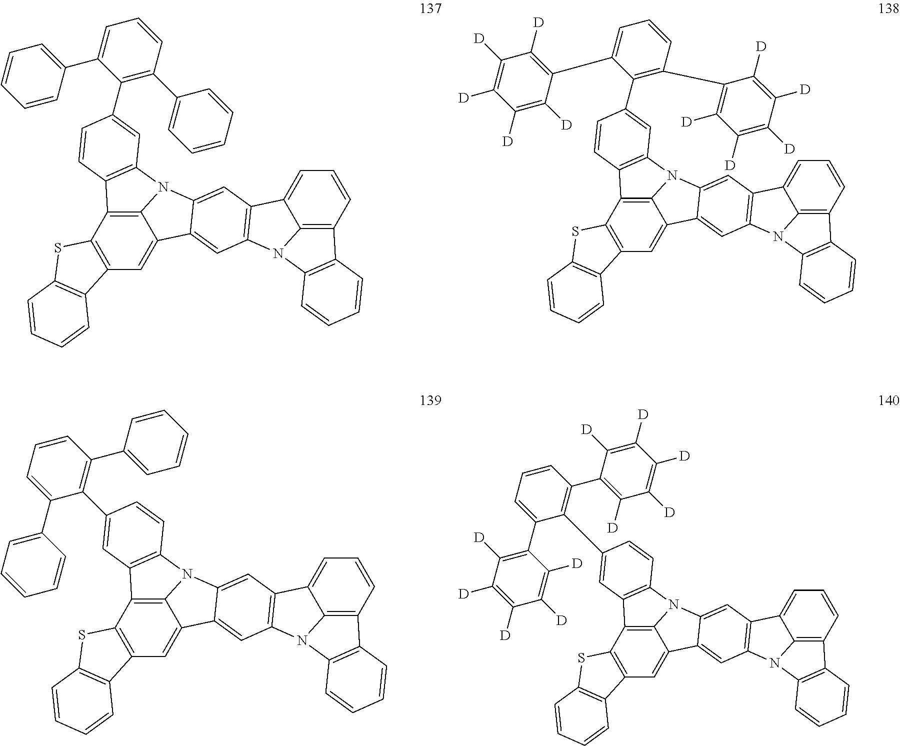

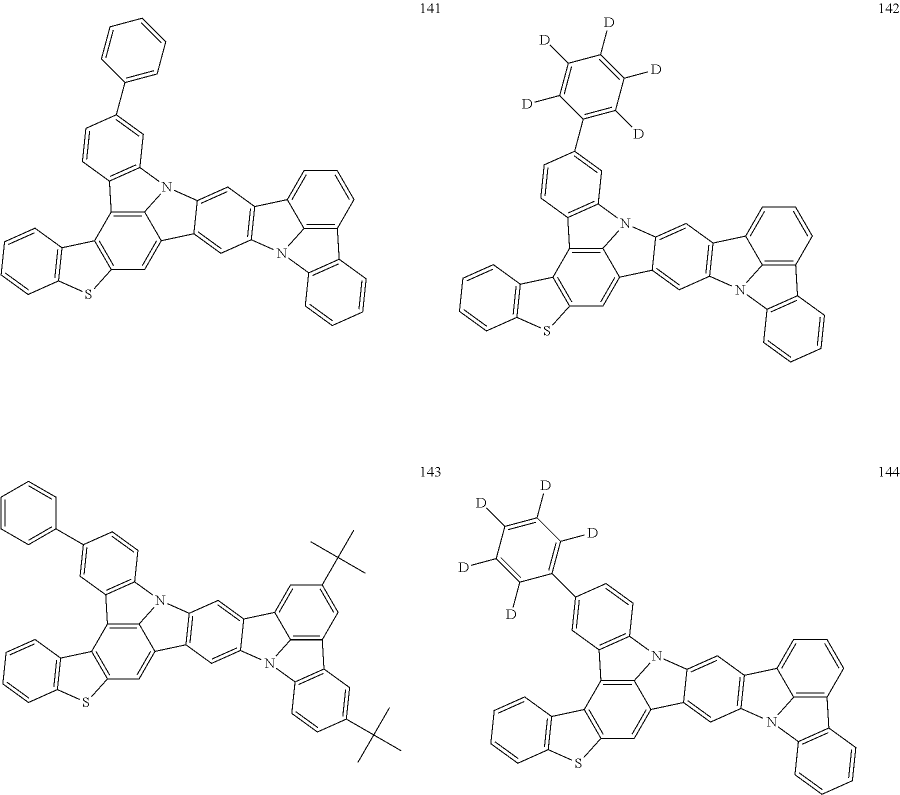 [SMILES: c1ccc(-c2ccc3c4c5c(cc6c7cc8c(cc7n(c3c2)c64)c2cccc3c4ccccc4n8c32)sc2ccccc25)cc1 ; [2H]c1c([2H])c([2H])c(-c2ccc3c4c5c(cc6c7cc8c(cc7n(c3c2)c64)c2cccc3c4ccccc4n8c32)sc2ccccc25)c([2H])c1[2H] ; CC(C)(C)c1ccc2c(c1)c1cc(C(C)(C)C)cc3c4cc5c(cc4n2c13)c1cc2sc3ccccc3c2c2c3cc(-c4ccccc4)ccc3n5c12 ; [2H]c1c([2H])c([2H])c(-c2ccc3c(c2)c2c4c(cc5c6cc7c(cc6n3c52)c2cccc3c5ccccc5n7c32)sc2ccccc24)c([2H])c1[2H]]

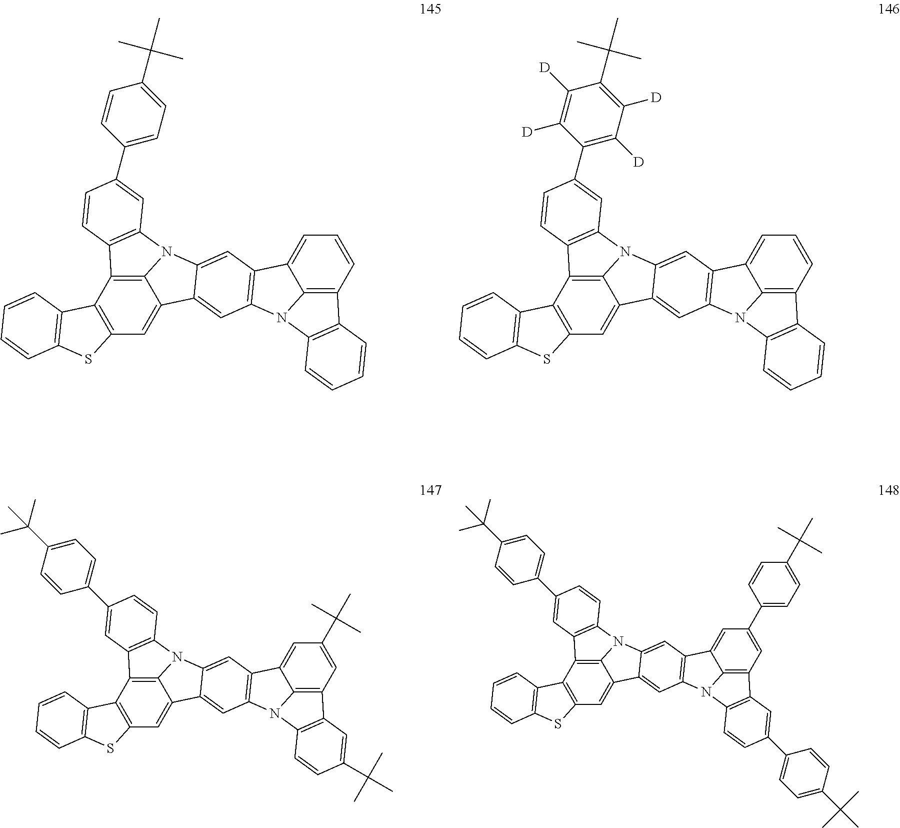 [SMILES: CC(C)(C)c1ccc(-c2ccc3c4c5c(cc6c7cc8c(cc7n(c3c2)c64)c2cccc3c4ccccc4n8c32)sc2ccccc25)cc1 ; [2H]c1c([2H])c(C(C)(C)C)c([2H])c([2H])c1-c1ccc2c3c4c(cc5c6cc7c(cc6n(c2c1)c53)c1cccc2c3ccccc3n7c21)sc1ccccc14 ; CC(C)(C)c1ccc(-c2ccc3c(c2)c2c4c(cc5c6cc7c(cc6n3c52)c2cc(C(C)(C)C)cc3c5cc(C(C)(C)C)ccc5n7c32)sc2ccccc24)cc1 ; CC(C)(C)c1ccc(-c2ccc3c(c2)c2cc(-c4ccc(C(C)(C)C)cc4)cc4c5cc6c(cc5n3c24)c2cc3sc4ccccc4c3c3c4cc(-c5ccc(C(C)(C)C)cc5)ccc4n6c23)cc1]

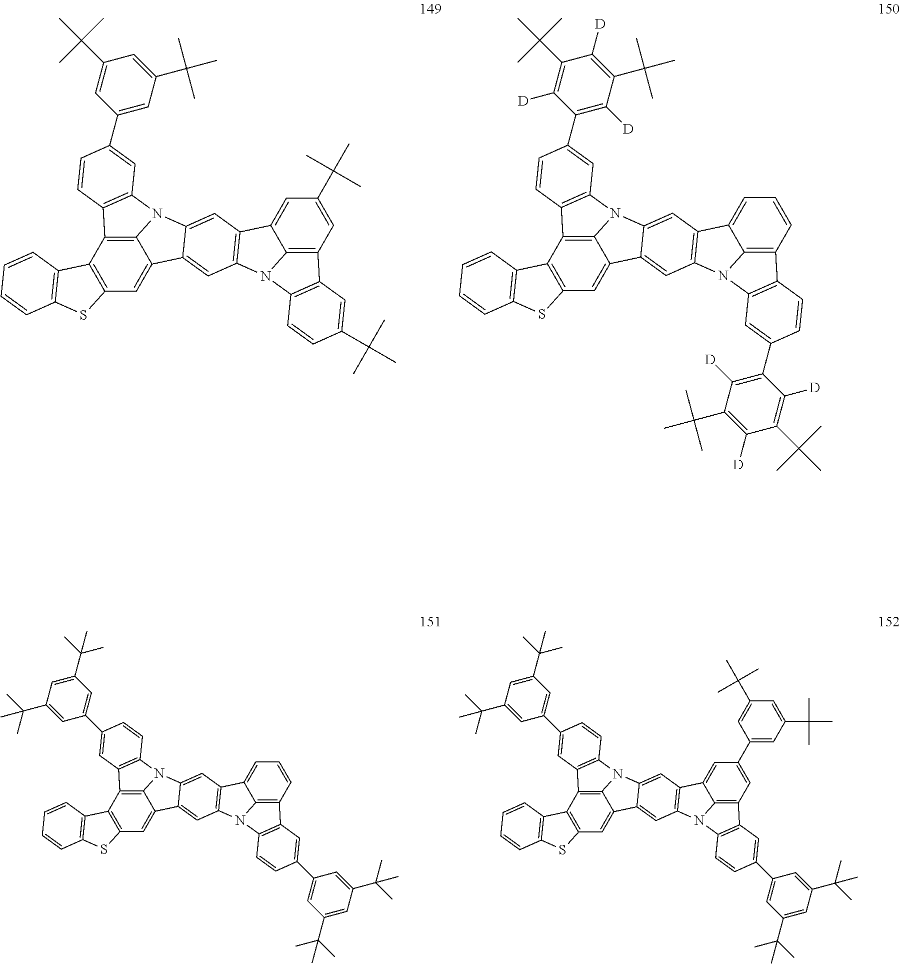

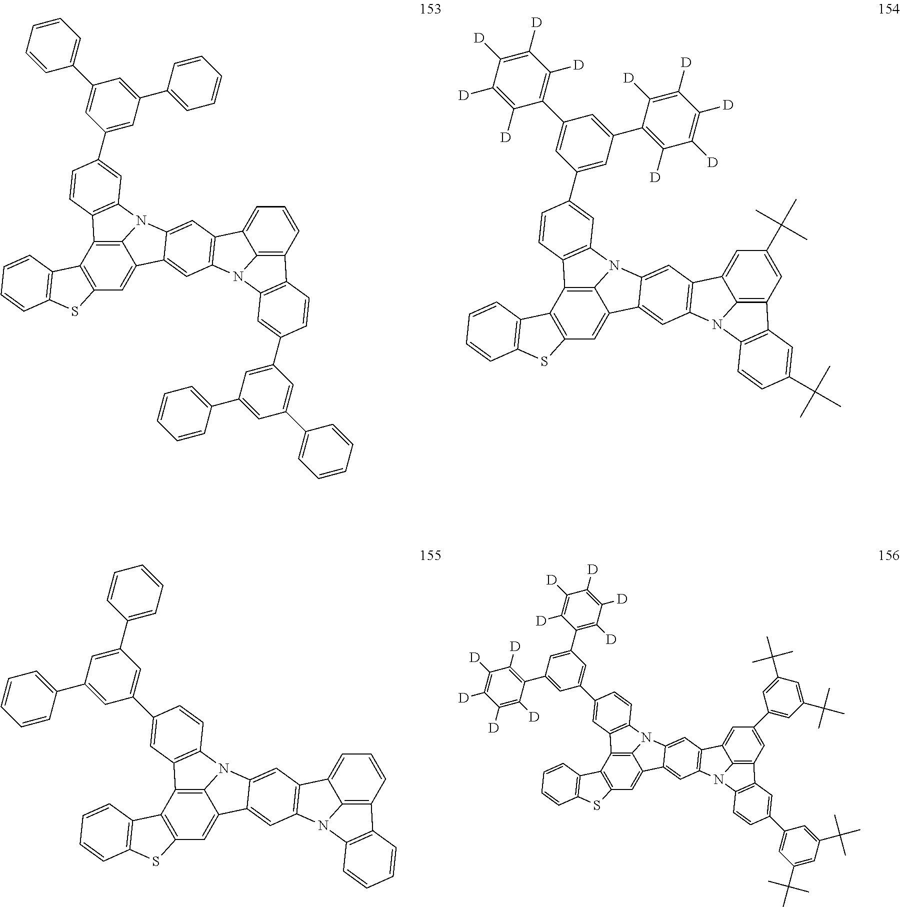

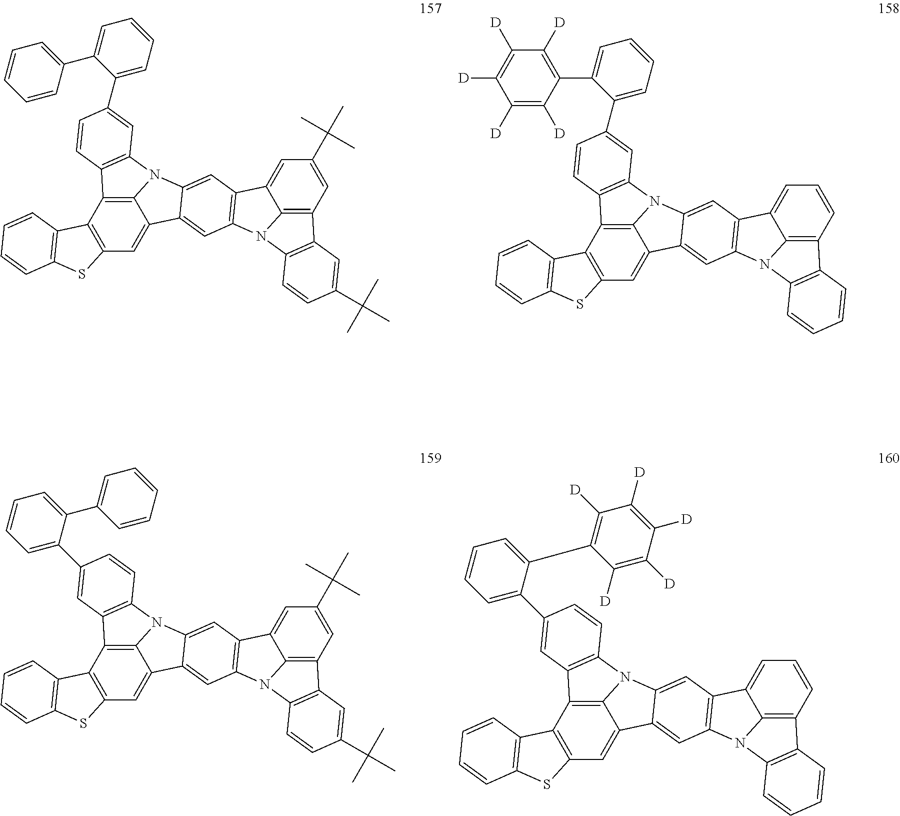 [SMILES: CC(C)(C)c1ccc2c(c1)c1cc(C(C)(C)C)cc3c4cc5c(cc4n2c13)c1cc2sc3ccccc3c2c2c3ccc(-c4ccccc4-c4ccccc4)cc3n5c12 ; [2H]c1c([2H])c([2H])c(-c2ccccc2-c2ccc3c4c5c(cc6c7cc8c(cc7n(c3c2)c64)c2cccc3c4ccccc4n8c32)sc2ccccc25)c([2H])c1[2H] ; CC(C)(C)c1ccc2c(c1)c1cc(C(C)(C)C)cc3c4cc5c(cc4n2c13)c1cc2sc3ccccc3c2c2c3cc(-c4ccccc4-c4ccccc4)ccc3n5c12 ; [2H]c1c([2H])c([2H])c(-c2ccccc2-c2ccc3c(c2)c2c4c(cc5c6cc7c(cc6n3c52)c2cccc3c5ccccc5n7c32)sc2ccccc24)c([2H])c1[2H]]

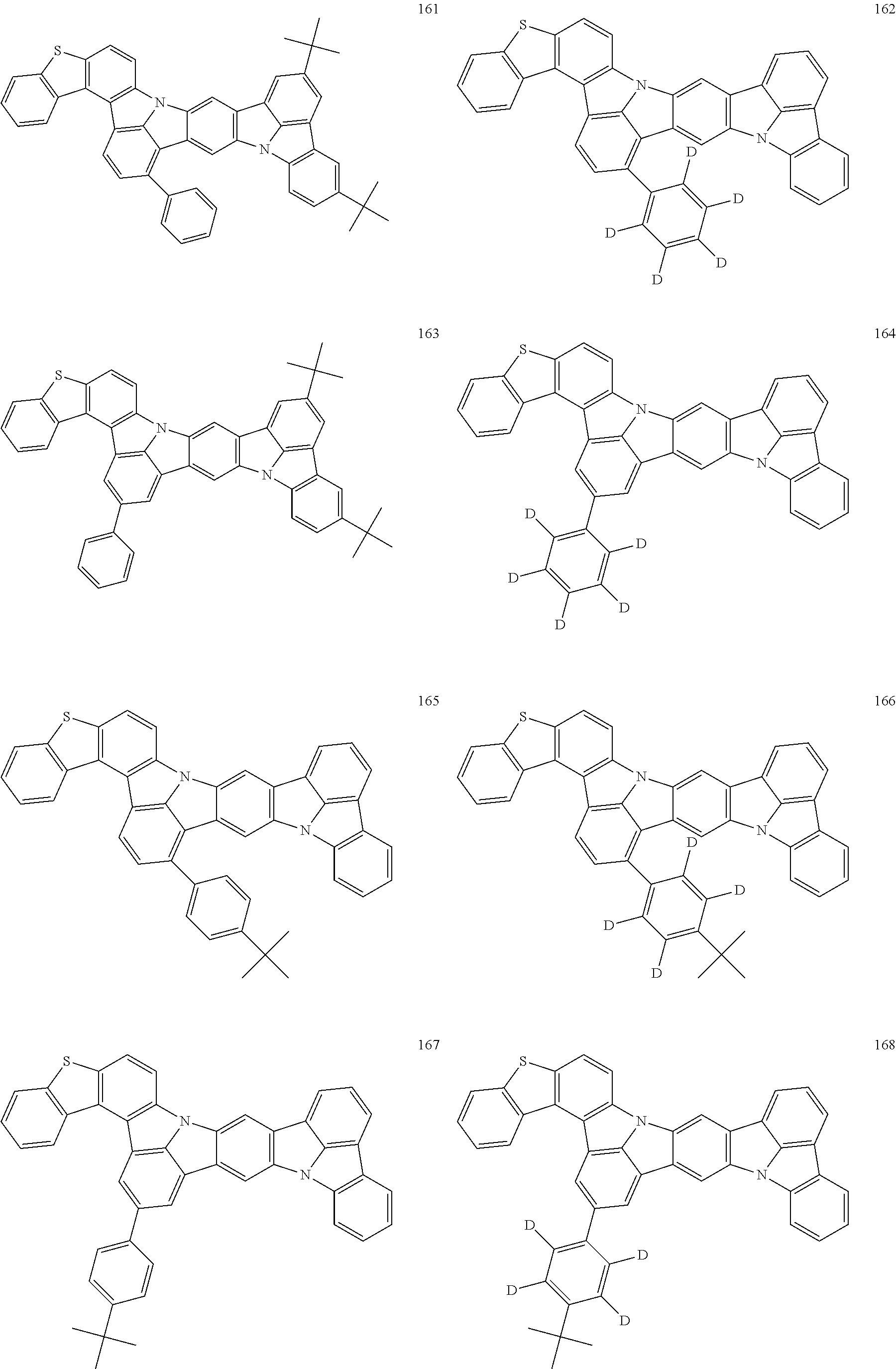

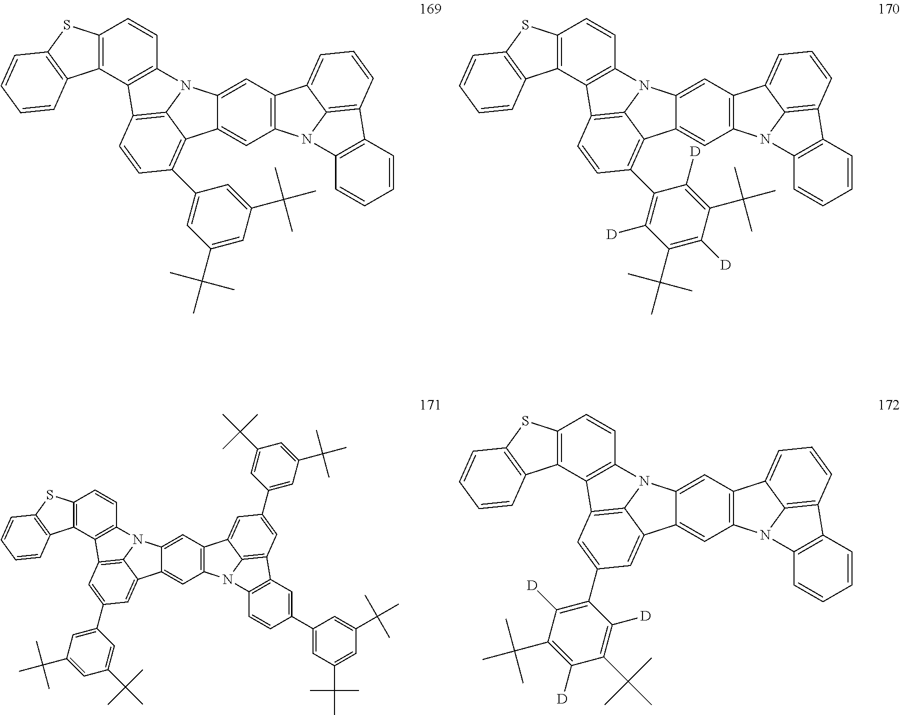 [SMILES: CC(C)(C)c1cc(-c2ccc3c4c5c(ccc4n4c6cc7c8cccc9c%10ccccc%10n(c7cc6c2c34)c98)sc2ccccc25)cc(C(C)(C)C)c1 ; [2H]c1c(-c2ccc3c4c5c(ccc4n4c6cc7c8cccc9c%10ccccc%10n(c7cc6c2c34)c98)sc2ccccc25)c([2H])c(C(C)(C)C)c([2H])c1C(C)(C)C ; CC(C)(C)c1cc(-c2ccc3c(c2)c2cc(-c4cc(C(C)(C)C)cc(C(C)(C)C)c4)cc4c5cc6c(cc5n3c24)c2cc(-c3cc(C(C)(C)C)cc(C(C)(C)C)c3)cc3c4c5c(ccc4n6c23)sc2ccccc25)cc(C(C)(C)C)c1 ; [2H]c1c(-c2cc3c4cc5c(cc4n4c6ccc7sc8ccccc8c7c6c(c2)c34)c2cccc3c4ccccc4n5c32)c([2H])c(C(C)(C)C)c([2H])c1C(C)(C)C]

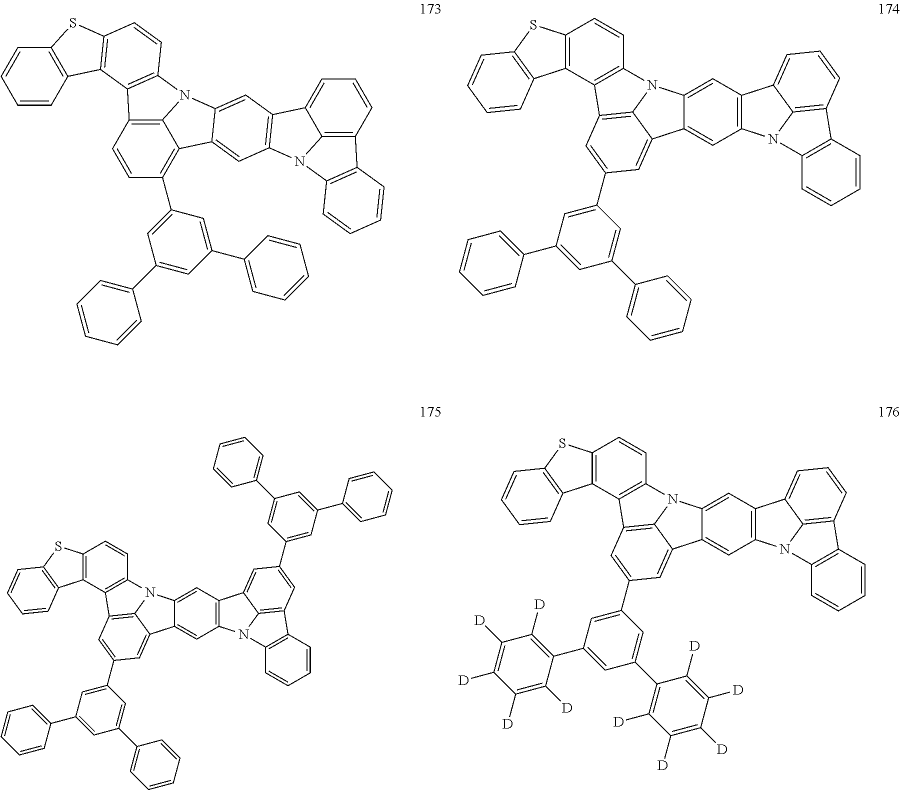

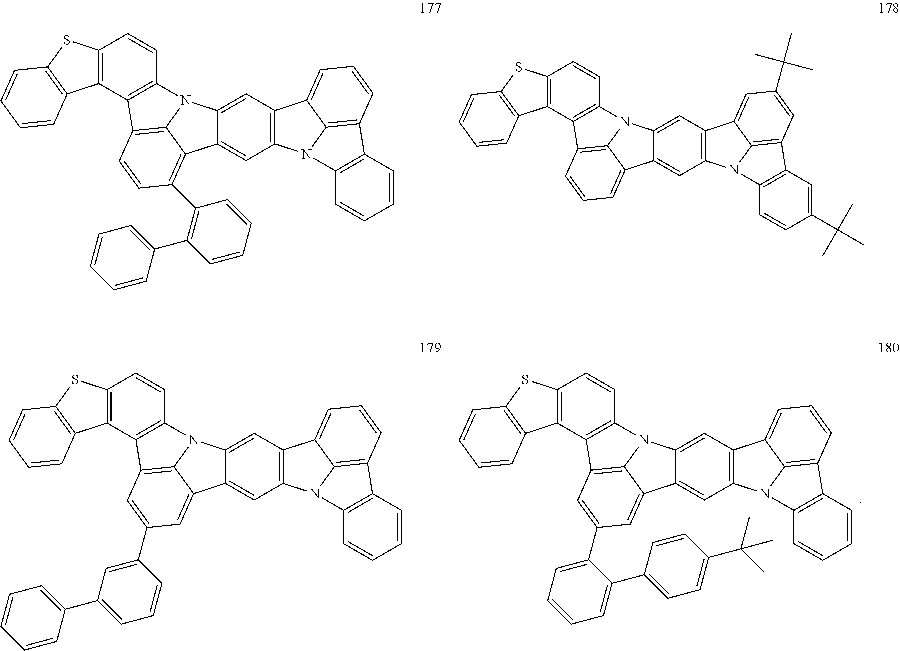 [SMILES: c1ccc(-c2ccccc2-c2ccc3c4c5c(ccc4n4c6cc7c8cccc9c%10ccccc%10n(c7cc6c2c34)c98)sc2ccccc25)cc1 ; CC(C)(C)c1ccc2c(c1)c1cc(C(C)(C)C)cc3c4cc5c(cc4n2c13)c1cccc2c3c4c(ccc3n5c12)sc1ccccc14 ; c1ccc(-c2cccc(-c3cc4c5cc6c(cc5n5c7ccc8sc9ccccc9c8c7c(c3)c45)c3cccc4c5ccccc5n6c43)c2)cc1 ; CC(C)(C)c1ccc(-c2ccccc2-c2cc3c4cc5c(cc4n4c6ccc7sc8ccccc8c7c6c(c2)c34)c2cccc3c4ccccc4n5c32)cc1]

In the embodiment compounds presented in Compound Group 1, “D” may refer to a deuterium atom.

The fused polycyclic compound represented by Formula 1 according to an embodiment includes a fused ring core in which a carbazole group is fused to an indolocarbazole moiety and has a structure in which at least one benzene ring is fused via a sulfur atom to the fused ring core, and thus high luminous efficiency and long service life may be achieved.

The fused polycyclic compound of an embodiment may have a structure in which a first carbazole group is fused to a first benzene ring from among first to third benzene rings constituting the indolocarbazole moiety. In this case, the nitrogen atom of the indolocarbazole group and the nitrogen atom of the first carbazole group may be linked at the para-position with respect to the first benzene ring. Accordingly, the fused polycyclic compound of an embodiment may be utilized as a delayed fluorescence emitting material by readily separating a highest occupied molecular orbital (HOMO) state and a lowest unoccupied molecular orbital (LUMO) states within one molecule by exhibiting multiple resonance in a broad planar skeleton. The fused polycyclic compound of an embodiment may have a full width of half maximum (FWHM) with a narrow spectrum due to the multiple resonance effect, and thus may provide a deep blue emission color with high color purity. In some embodiments, the fused polycyclic compound of an embodiment may have an improvement in device service life characteristics due to increased material stability as compared with a related art polycyclic compound containing a boron atom. The boron atom may have electron deficiency characteristics by an empty p-orbital, thereby form a bond with other nucleophiles, and thus be changed from a trigonal planar structure into a tetrahedral structure, which may cause deterioration of the device. According to the present disclosure, the fused polycyclic compound represented by Formula 1 does not contain a boron atom, thereby has high material stability, and thus may suppress or reduce a decrease in service life due to the deterioration phenomenon.

The fused polycyclic compound of an embodiment may include a structure in which at least one structure of Formula 2 containing one sulfur atom and one benzene ring is fused at a central fused ring core. The fused polycyclic compound of an embodiment includes a structure in which at least one substituent represented by Formula 2 is fused at the fused ring core, thereby triplet excitons may be harvested rapidly from singlet excitons through reverse intersystem crossing mechanism, and thus when the fused polycyclic compound is utilized as a delayed fluorescence emitting material, the luminous efficiency of the light emitting device may be improved. In the case of the polycyclic compound in which a carbazole group is fused to an indolocarbazole moiety, the difference (ΔEst) between a lowest triplet exciton energy level (T1 level) and a lowest singlet exciton energy level (S1 level) is higher than that of a boron-based polycyclic compound, and thus there may be a disadvantage in terms of the reverse intersystem crossing. According to the present disclosure, at least one substituent represented by Formula 2 containing a sulfur atom is fused at the fused ring core, thereby the fused polycyclic compound of an embodiment may have an improvement in spin-orbit coupling (SOC), and thus the speed of the reverse intersystem crossing is increased so that the luminous efficiency may be increased. For example, the fused polycyclic compound of an embodiment may have further improvement in the luminous efficiency due to the amplified speed of the reverse intersystem crossing even though the difference between the lowest triplet exciton energy level and the lowest singlet exciton energy level is somewhat large.

In some embodiments, the fused polycyclic compound of an embodiment may be included in the emission layer EML. The fused polycyclic compound of an embodiment may be included as a dopant material in the emission layer EML. The fused polycyclic compound of an embodiment may be a thermally activated delayed fluorescence material. The fused polycyclic compound of an embodiment may be utilized as a thermally activated delayed fluorescence dopant. For example, in the light emitting device ED of an embodiment, the emission layer EML may include, as a thermally activated delayed fluorescence dopant, at least one from among the fused polycyclic compounds represented by Compound Group 1 as described above. However, a usage of the fused polycyclic compound of an embodiment is not limited thereto.

In an embodiment, the emission layer EML may include a plurality of compounds. The emission layer EML of an embodiment may include the fused polycyclic compound represented by Formula 1, i.e., the first compound, and at least one of the second compound represented by Formula H-1, the third compound represented by Formula H-2, or the fourth compound represented by Formula D-1:

In an embodiment, the emission layer EML may include the second compound represented by Formula H-1. In an embodiment, the second compound may be utilized as a hole transporting host material of the emission layer EML.

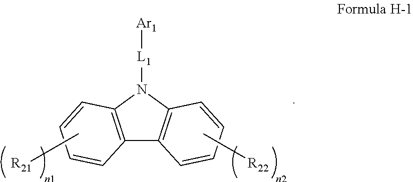

In Formula H-1, L₁ may be a direct linkage, a substituted or unsubstituted arylene group having 6 to 30 ring-forming carbon atoms, or a substituted or unsubstituted heteroarylene group having 2 to 30 ring-forming carbon atoms. For example, L₁ may be a direct linkage, a substituted or unsubstituted phenylene group, a substituted or unsubstituted divalent biphenyl group, a substituted or unsubstituted divalent carbazole group, etc., but the embodiment of the present disclosure is not limited thereto.

In Formula H-1, An may be a substituted or unsubstituted aryl group having 6 to 30 ring-forming carbon atoms, or a substituted or unsubstituted heteroaryl group having 2 to 30 ring-forming carbon atoms. For example, An may be a substituted or unsubstituted carbazole group, a substituted or unsubstituted dibenzofuran group, a substituted or unsubstituted dibenzothiophene group, a substituted or unsubstituted biphenyl group, etc., but the embodiment of the present disclosure is not limited thereto.

In Formula H-1, R₂₁ and R₂₂ may each independently be a hydrogen atom, a deuterium atom, a halogen atom, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group having 1 to 20 carbon atoms, a substituted or unsubstituted alkenyl group having 2 to 20 carbon atoms, a substituted or unsubstituted aryl group having 6 to 60 ring-forming carbon atoms, or a substituted or unsubstituted heteroaryl group having 2 to 60 ring-forming carbon atoms. In some embodiments, each of R₂₁ and R₂₂ may be bonded to an adjacent group to form a ring. For example, R₂₁ and R₂₂ may each independently be a hydrogen atom or a deuterium atom.

In Formula H-1, n1 and n2 may each independently be an integer of 0 to 4. When n1 and/or n2 is 0, the fused polycyclic compound of an embodiment may not be substituted with R₂₁ and/or R₂₂. The case where n1 and/or n2 is 4 and R₂₁'s and/or R₂₂'s are each hydrogen atoms may be the same as the case where n1 and/or n2 is 0. When n1 and/or n2 is an integer of 2 or more, a plurality of R₂₁'s and/or R₂₂'s may each be the same or at least one from among the plurality of R₂₁'s and/or R₂₂'s may be different from the others.

In an embodiment, the second compound represented by Formula 2 may be represented by any one from among the compounds represented by Compound Group 2. The emission layer EML may include at least one from among the compounds represented by Compound Group 2 as a hole transporting host material.

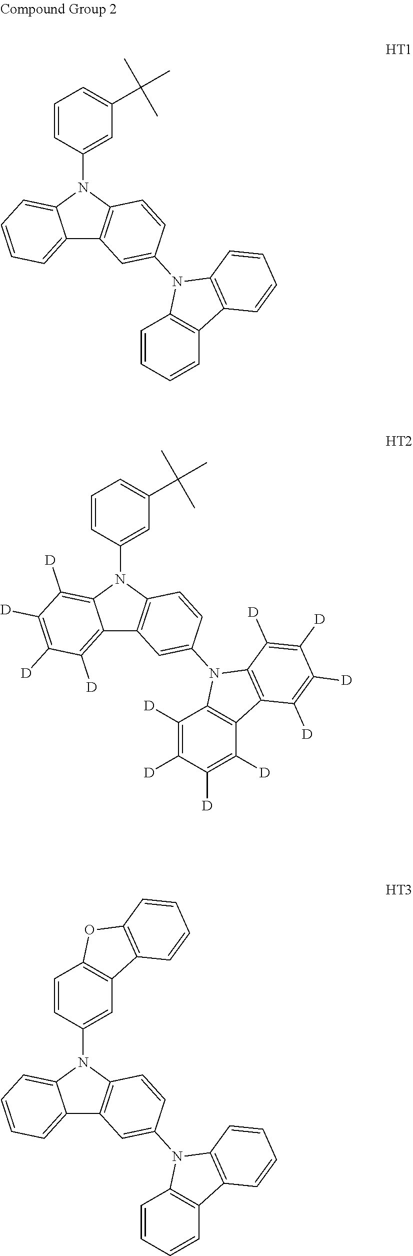 [SMILES: CC(C)(C)c1cccc(-n2c3ccccc3c3cc(-n4c5ccccc5c5ccccc54)ccc32)c1 ; [2H]c1c([2H])c([2H])c2c(c1[2H])c1cc(-n3c4c([2H])c([2H])c([2H])c([2H])c4c4c([2H])c([2H])c([2H])c([2H])c43)ccc1n2-c1cccc(C(C)(C)C)c1 ; c1ccc2c(c1)oc1ccc(-n3c4ccccc4c4cc(-n5c6ccccc6c6ccccc65)ccc43)cc12]

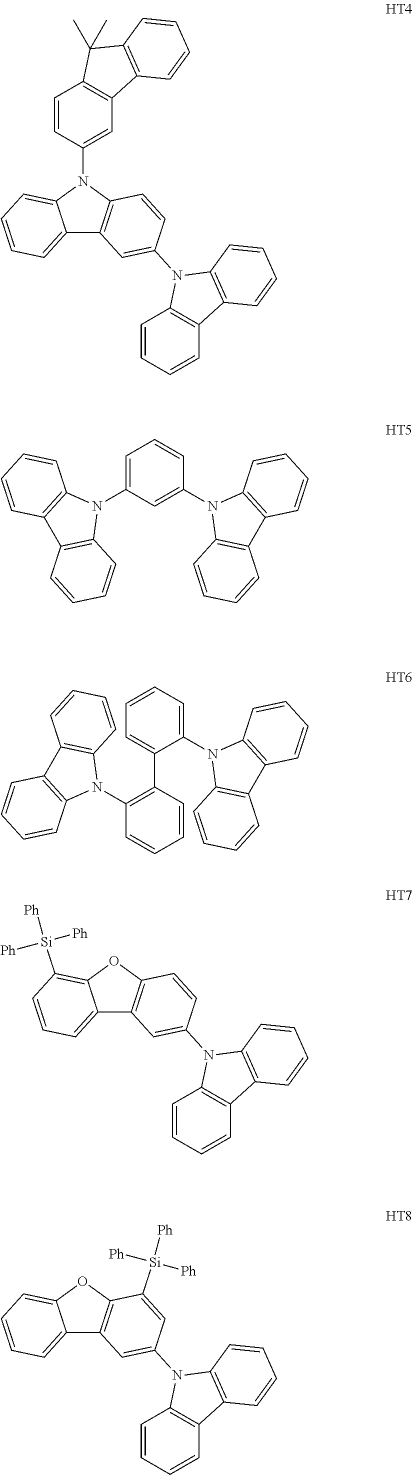 [SMILES: CC1(C)c2ccccc2-c2cc(-n3c4ccccc4c4cc(-n5c6ccccc6c6ccccc65)ccc43)ccc21 ; c1cc(-n2c3ccccc3c3ccccc32)cc(-n2c3ccccc3c3ccccc32)c1 ; c1ccc(-n2c3ccccc3c3ccccc32)c(-c2ccccc2-n2c3ccccc3c3ccccc32)c1 ; c1ccc([Si](c2ccccc2)(c2ccccc2)c2cccc3c2oc2ccc(-n4c5ccccc5c5ccccc54)cc23)cc1 ; c1ccc([Si](c2ccccc2)(c2ccccc2)c2cc(-n3c4ccccc4c4ccccc43)cc3c2oc2ccccc23)cc1]

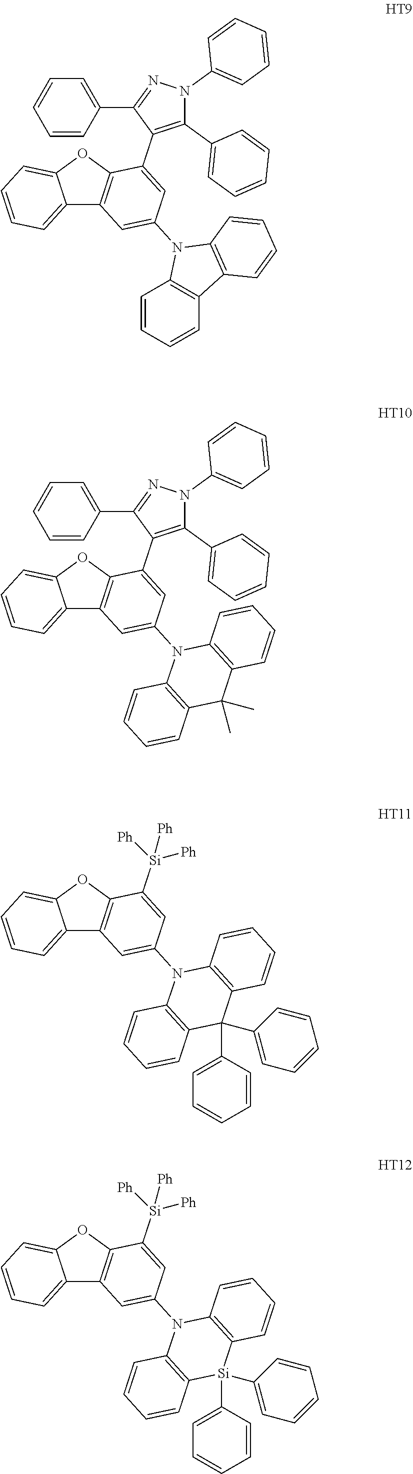 [SMILES: c1ccc(-c2nn(-c3ccccc3)c(-c3ccccc3)c2-c2cc(-n3c4ccccc4c4ccccc43)cc3c2oc2ccccc23)cc1 ; CC1(C)c2ccccc2N(c2cc(-c3c(-c4ccccc4)nn(-c4ccccc4)c3-c3ccccc3)c3oc4ccccc4c3c2)c2ccccc21 ; c1ccc(C2(c3ccccc3)c3ccccc3N(c3cc([Si](c4ccccc4)(c4ccccc4)c4ccccc4)c4oc5ccccc5c4c3)c3ccccc32)cc1 ; c1ccc([Si]2(c3ccccc3)c3ccccc3N(c3cc([Si](c4ccccc4)(c4ccccc4)c4ccccc4)c4oc5ccccc5c4c3)c3ccccc32)cc1]

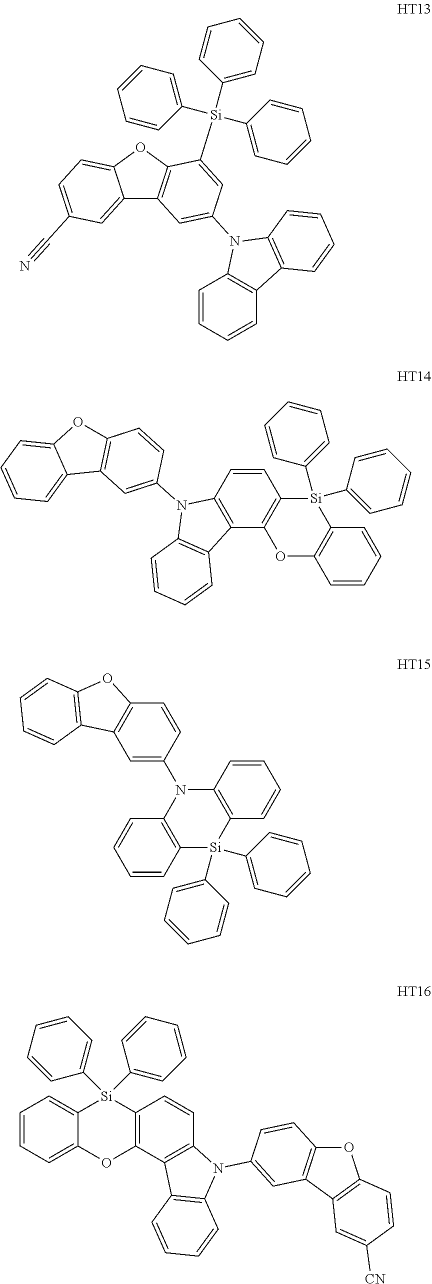 [SMILES: N#Cc1ccc2oc3c([Si](c4ccccc4)(c4ccccc4)c4ccccc4)cc(-n4c5ccccc5c5ccccc54)cc3c2c1 ; c1ccc([Si]2(c3ccccc3)c3ccccc3Oc3c2ccc2c3c3ccccc3n2-c2ccc3oc4ccccc4c3c2)cc1 ; c1ccc([Si]2(c3ccccc3)c3ccccc3N(c3ccc4oc5ccccc5c4c3)c3ccccc32)cc1 ; N#Cc1ccc2oc3ccc(-n4c5ccccc5c5c6c(ccc54)[Si](c4ccccc4)(c4ccccc4)c4ccccc4O6)cc3c2c1]

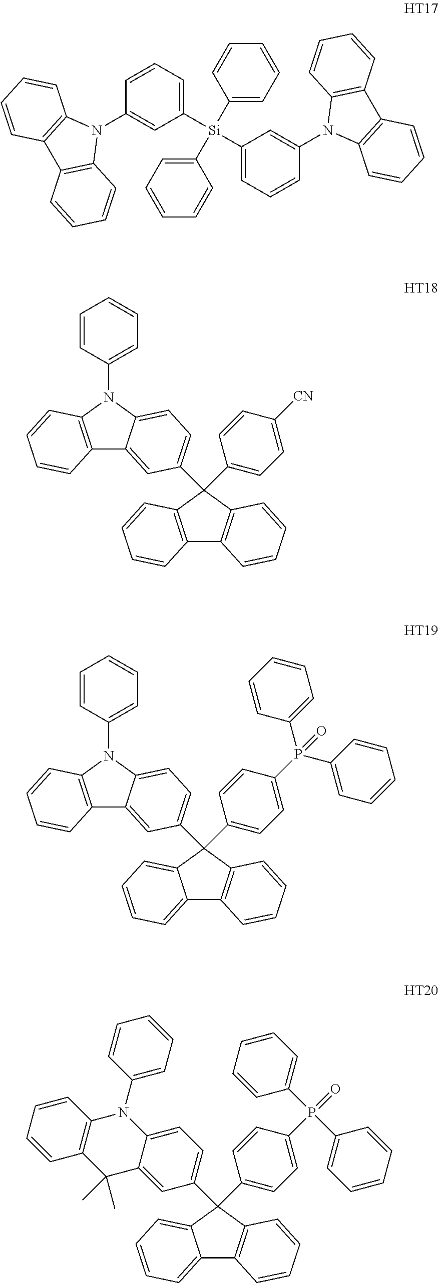 [SMILES: c1ccc([Si](c2ccccc2)(c2cccc(-n3c4ccccc4c4ccccc43)c2)c2cccc(-n3c4ccccc4c4ccccc43)c2)cc1 ; N#Cc1ccc(C2(c3ccc4c(c3)c3ccccc3n4-c3ccccc3)c3ccccc3-c3ccccc32)cc1 ; O=P(c1ccccc1)(c1ccccc1)c1ccc(C2(c3ccc4c(c3)c3ccccc3n4-c3ccccc3)c3ccccc3-c3ccccc32)cc1 ; CC1(C)c2ccccc2N(c2ccccc2)c2ccc(C3(c4ccc(P(=O)(c5ccccc5)c5ccccc5)cc4)c4ccccc4-c4ccccc43)cc21]

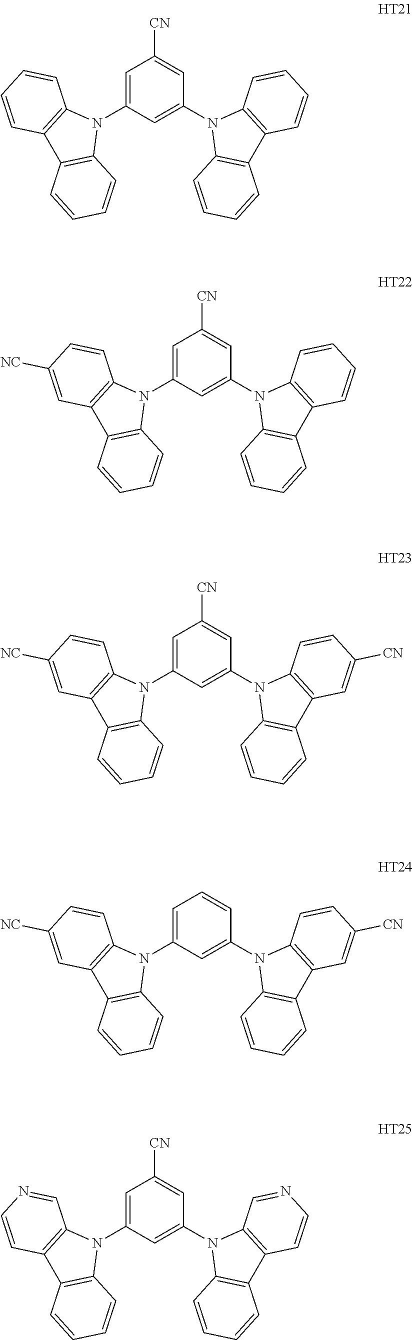 [SMILES: N#Cc1cc(-n2c3ccccc3c3ccccc32)cc(-n2c3ccccc3c3ccccc32)c1 ; N#Cc1cc(-n2c3ccccc3c3ccccc32)cc(-n2c3ccccc3c3cc(C#N)ccc32)c1 ; N#Cc1cc(-n2c3ccccc3c3cc(C#N)ccc32)cc(-n2c3ccccc3c3cc(C#N)ccc32)c1 ; N#Cc1ccc2c(c1)c1ccccc1n2-c1cccc(-n2c3ccccc3c3cc(C#N)ccc32)c1 ; N#Cc1cc(-n2c3ccccc3c3ccncc32)cc(-n2c3ccccc3c3ccncc32)c1]

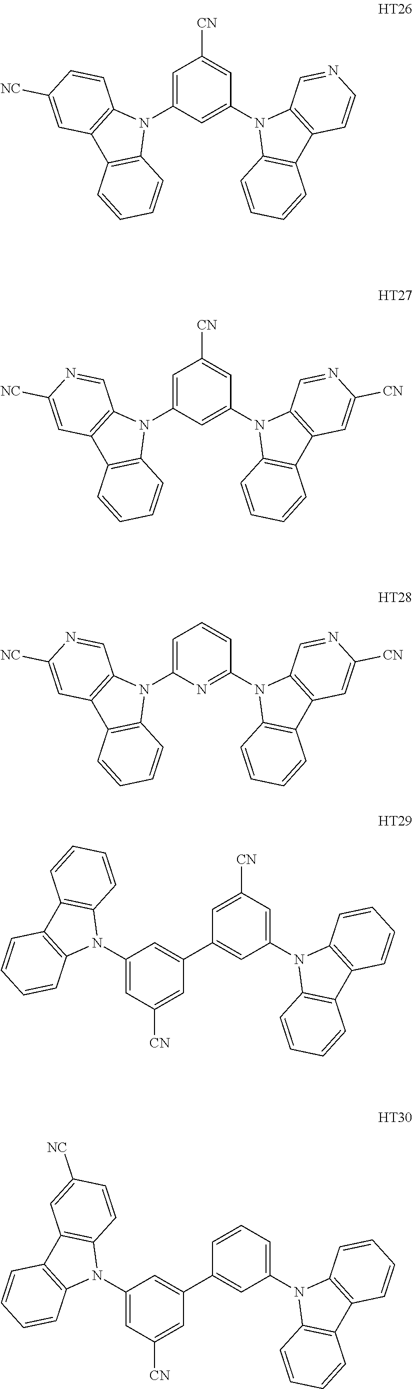 [SMILES: N#Cc1cc(-n2c3ccccc3c3cc(C#N)ccc32)cc(-n2c3ccccc3c3ccncc32)c1 ; N#Cc1cc(-n2c3ccccc3c3cc(C#N)ncc32)cc(-n2c3ccccc3c3cc(C#N)ncc32)c1 ; N#Cc1cc2c3ccccc3n(-c3cccc(-n4c5ccccc5c5cc(C#N)ncc54)n3)c2cn1 ; N#Cc1cc(-c2cc(C#N)cc(-n3c4ccccc4c4ccccc43)c2)cc(-n2c3ccccc3c3ccccc32)c1 ; N#Cc1cc(-c2cccc(-n3c4ccccc4c4ccccc43)c2)cc(-n2c3ccccc3c3cc(C#N)ccc32)c1]

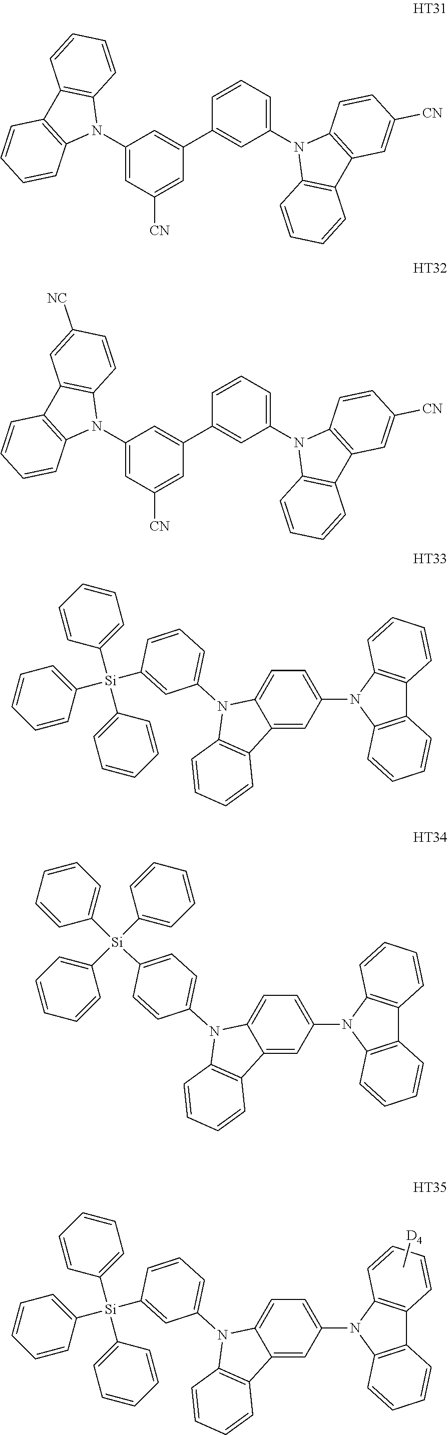

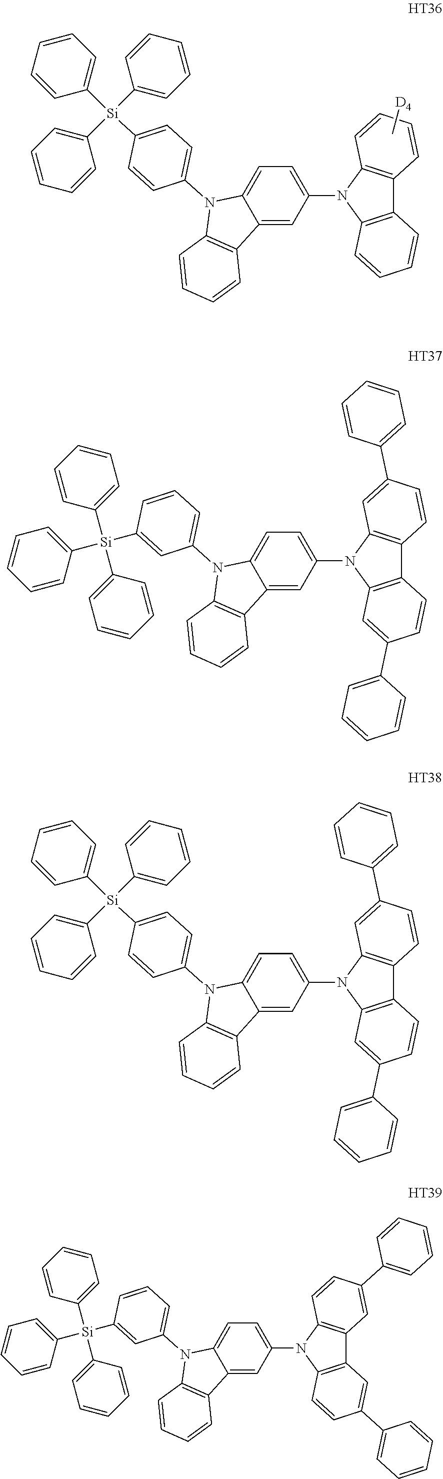

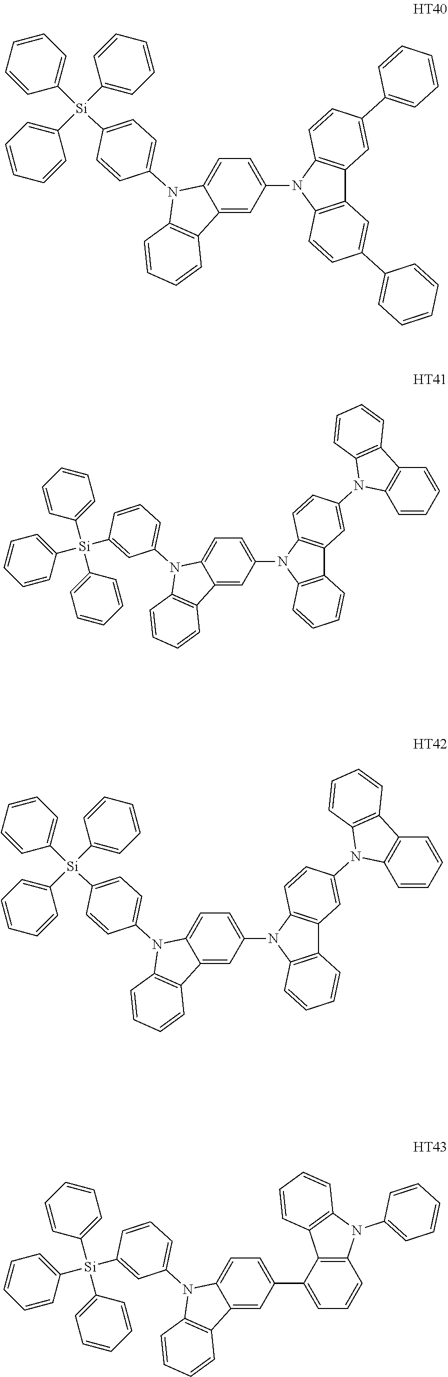 [SMILES: c1ccc(-c2ccc3c(c2)c2cc(-c4ccccc4)ccc2n3-c2ccc3c(c2)c2ccccc2n3-c2ccc([Si](c3ccccc3)(c3ccccc3)c3ccccc3)cc2)cc1 ; c1ccc([Si](c2ccccc2)(c2ccccc2)c2cccc(-n3c4ccccc4c4cc(-n5c6ccccc6c6cc(-n7c8ccccc8c8ccccc87)ccc65)ccc43)c2)cc1 ; c1ccc([Si](c2ccccc2)(c2ccccc2)c2ccc(-n3c4ccccc4c4cc(-n5c6ccccc6c6cc(-n7c8ccccc8c8ccccc87)ccc65)ccc43)cc2)cc1 ; c1ccc(-n2c3ccccc3c3c(-c4ccc5c(c4)c4ccccc4n5-c4cccc([Si](c5ccccc5)(c5ccccc5)c5ccccc5)c4)cccc32)cc1]

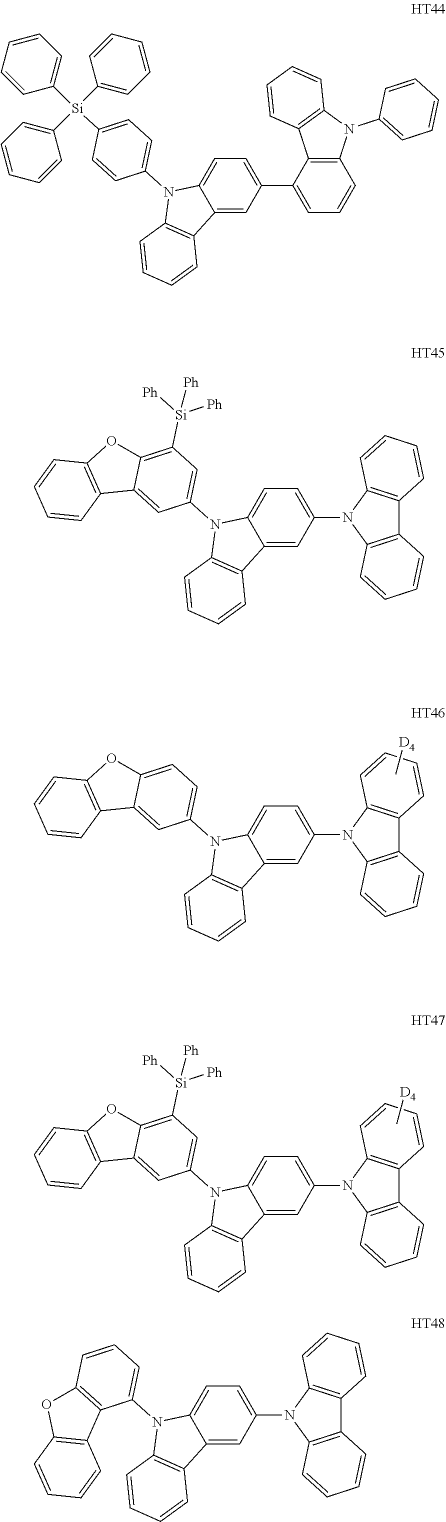

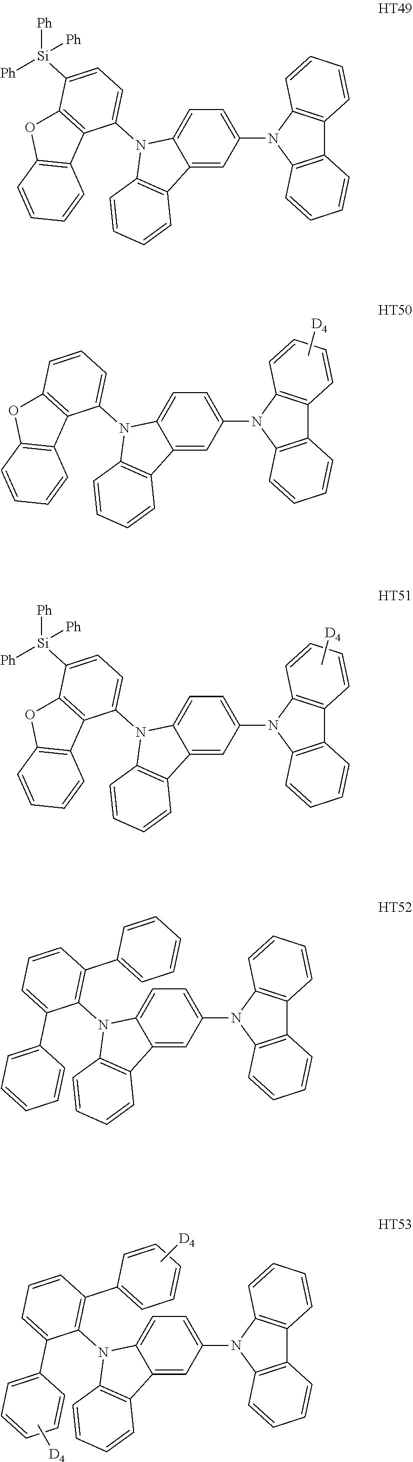

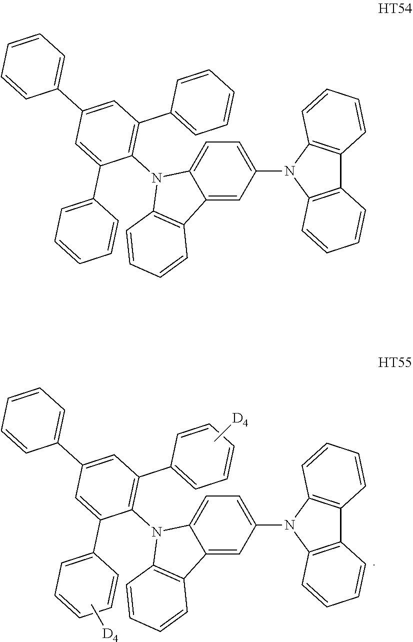

In embodiment compounds presented in Compound Group 2, “D” may refer to a deuterium atom, and “Ph” may refer to a substituted or unsubstituted phenyl group. For example, in example compounds presented in Compound Group 2, “Ph” may refer to an unsubstituted phenyl group.

In an embodiment, the emission layer EML may include the third compound represented by Formula H-2. For example, the third compound may be utilized as an electron transport host material of the emission layer EML.

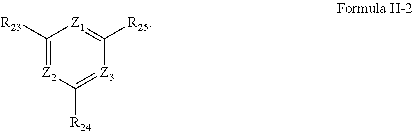

In Formula H-2, Z₁ to Z₃ may each independently be N or CR₂₆, and at least one from among Z₁ to Z₃ may be N.

In Formula H-2, R₂₃ to R₂₆ may each independently be a hydrogen atom, a deuterium atom, a halogen atom, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group having 1 to 20 carbon atoms, a substituted or unsubstituted alkenyl group having 2 to 20 carbon atoms, a substituted or unsubstituted aryl group having 6 to 60 ring-forming carbon atoms, or a substituted or unsubstituted heteroaryl group having 2 to 60 ring-forming carbon atoms. In some embodiments, each of R₂₃ to R₂₆ may be bonded to an adjacent group to form a ring. For example, R₂₃ to R₂₆ may each independently be a substituted or unsubstituted phenyl group, a substituted or unsubstituted carbazole group, etc., but the embodiment of the present disclosure is not limited thereto.

In an embodiment, the third compound represented by Formula 3 may be represented by any one from among the compounds represented by Compound Group 3. The emission layer EML may include at least one from among the compounds represented by Compound Group 3 as an electron transporting host material.

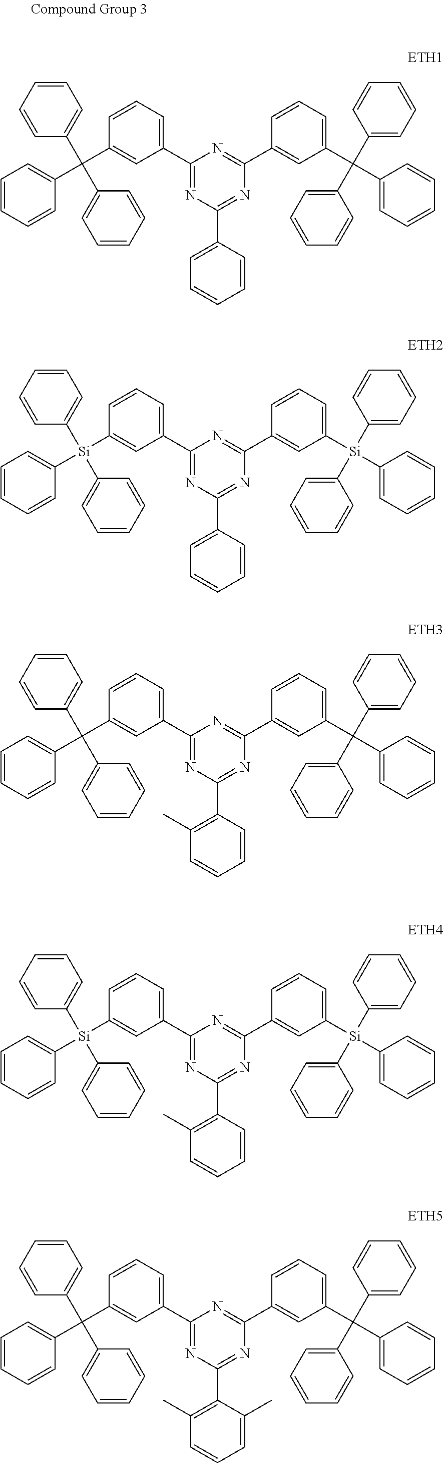

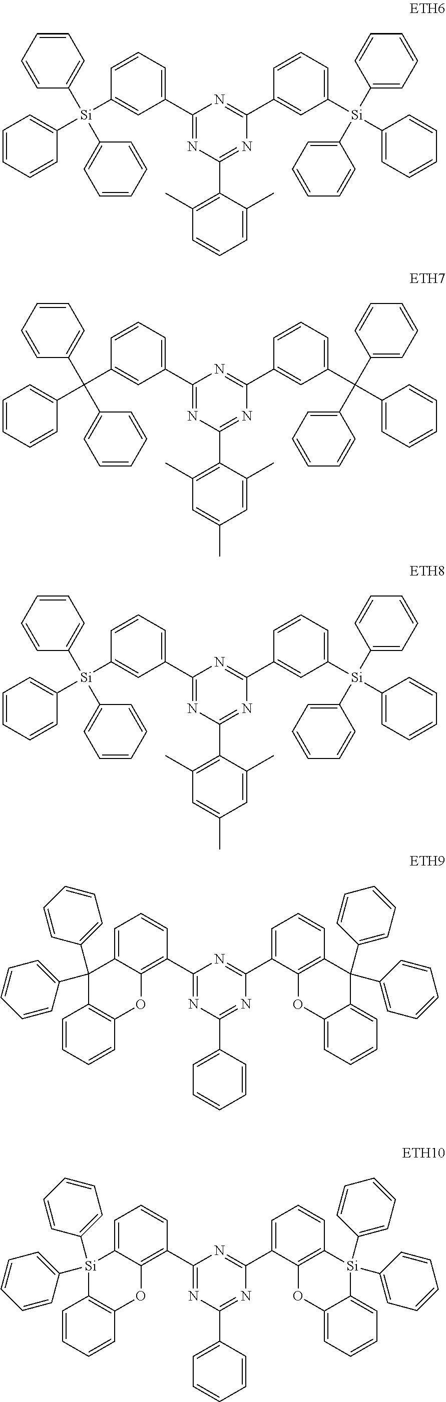

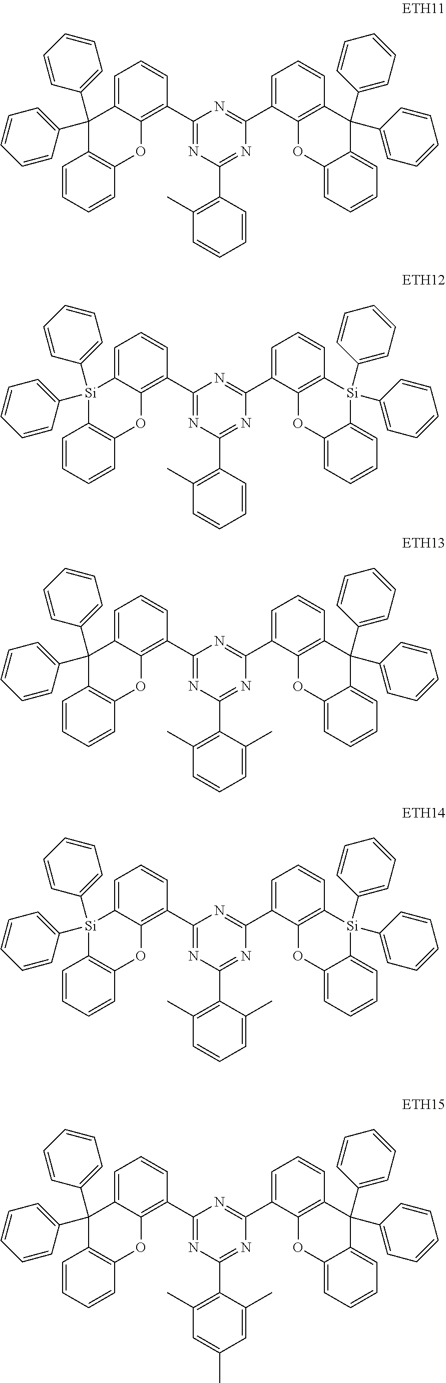

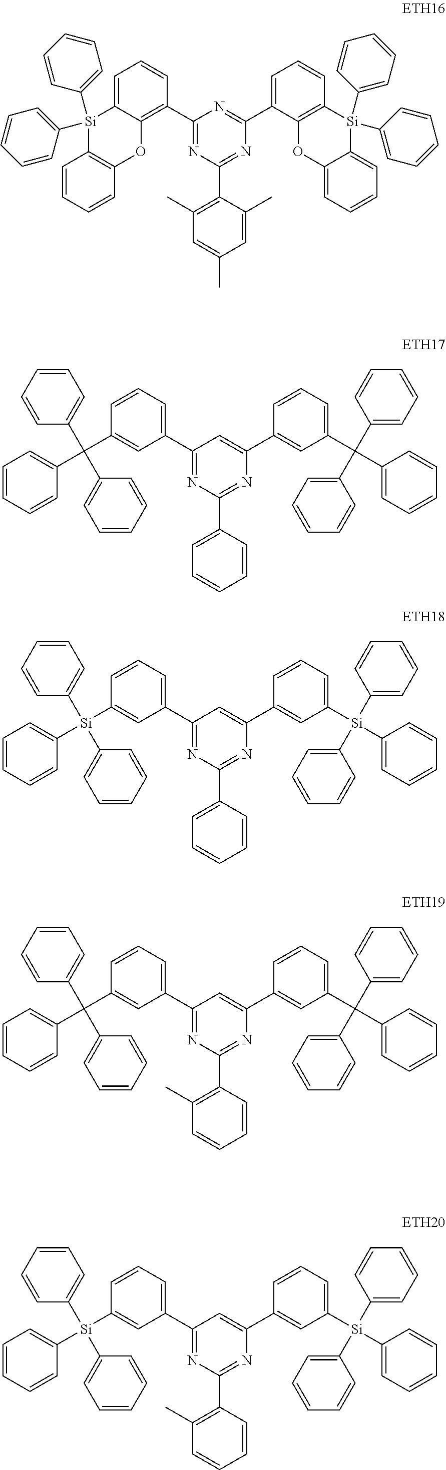

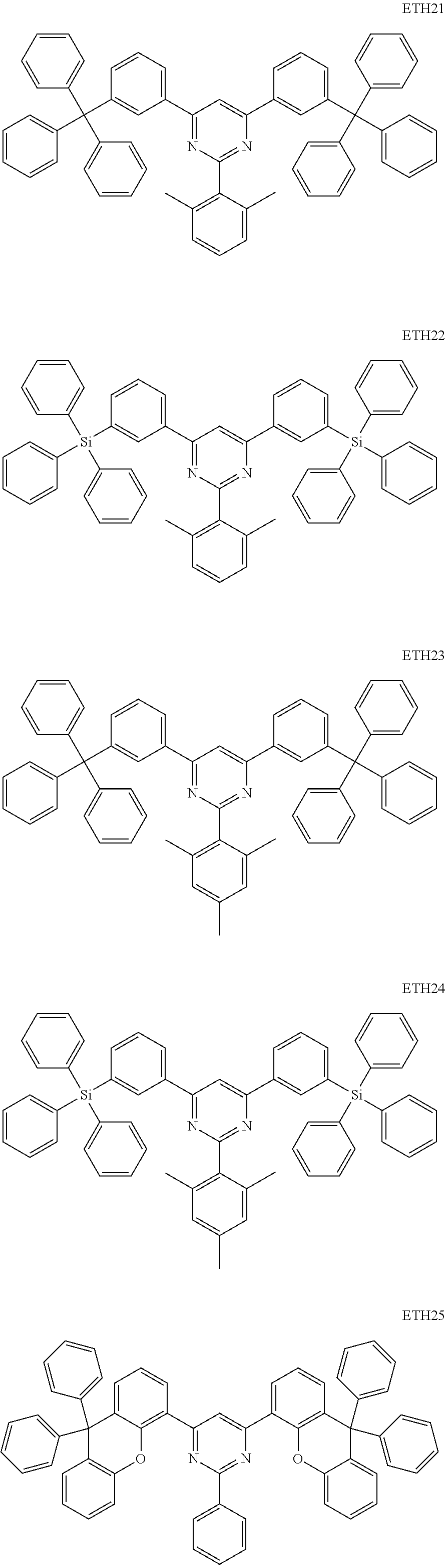

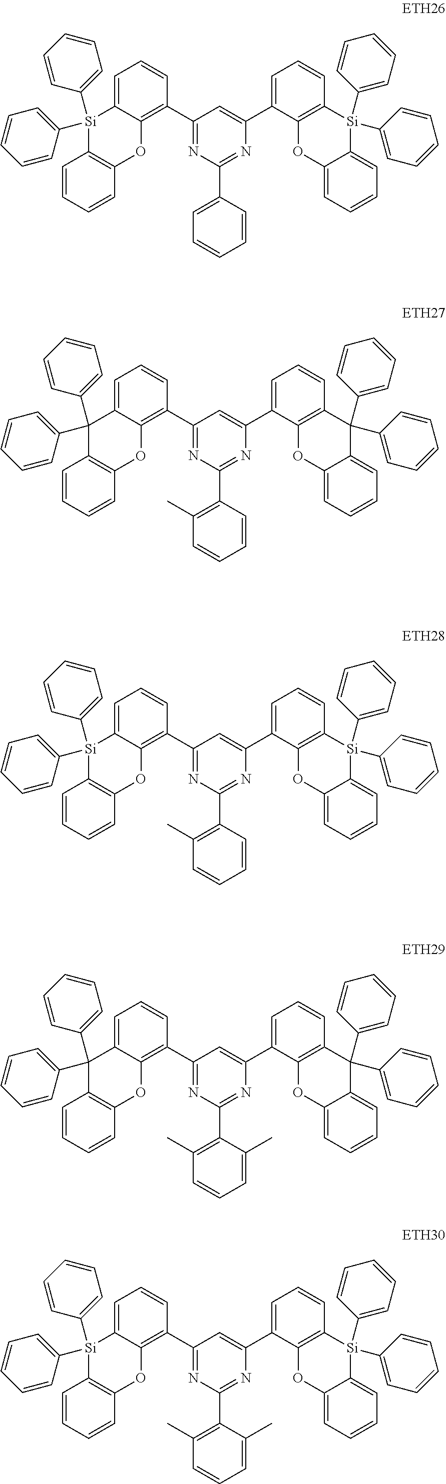

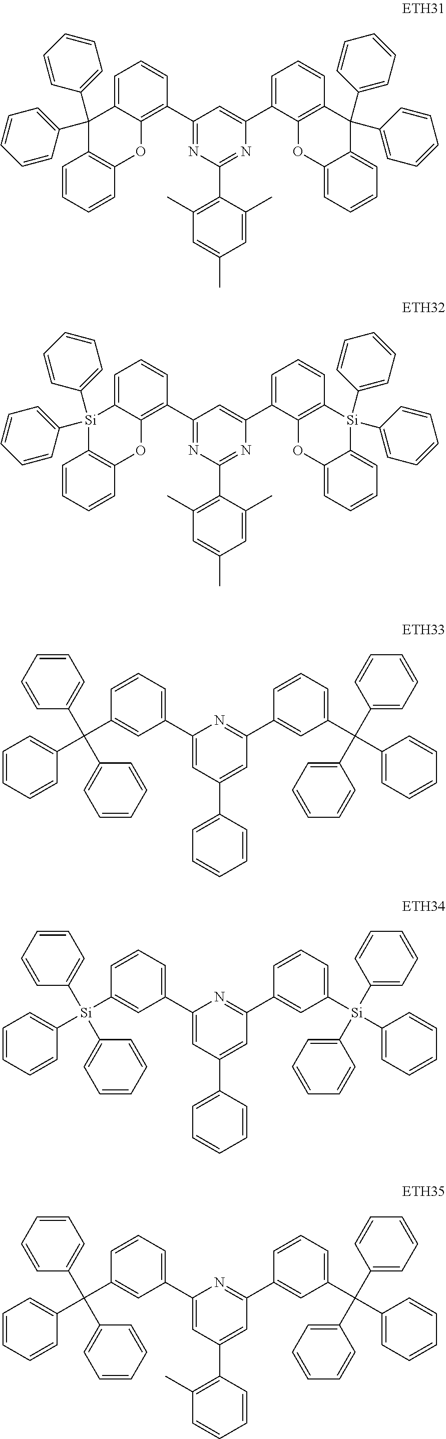

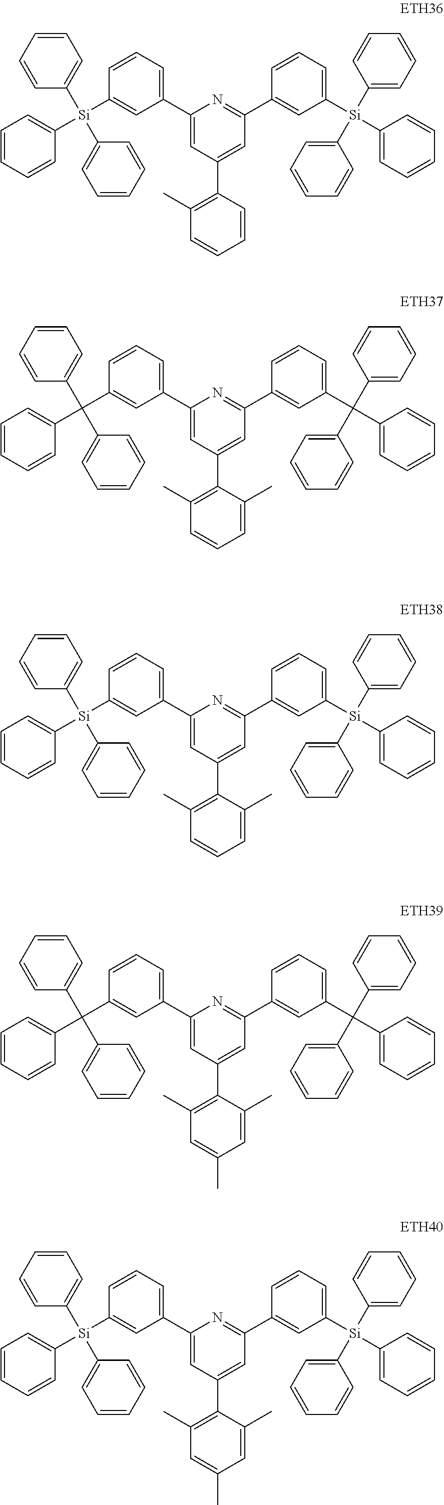

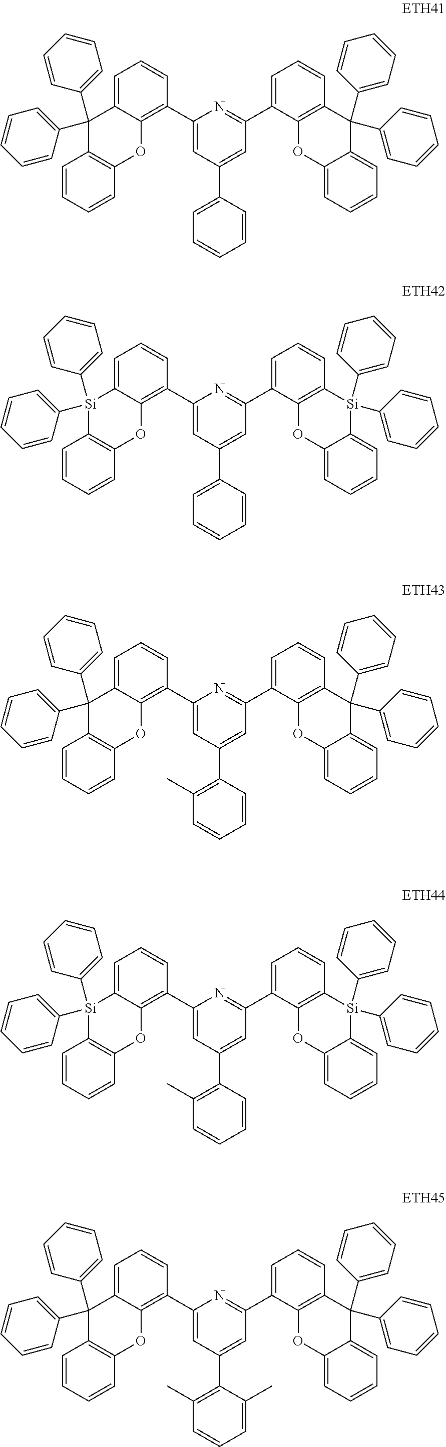

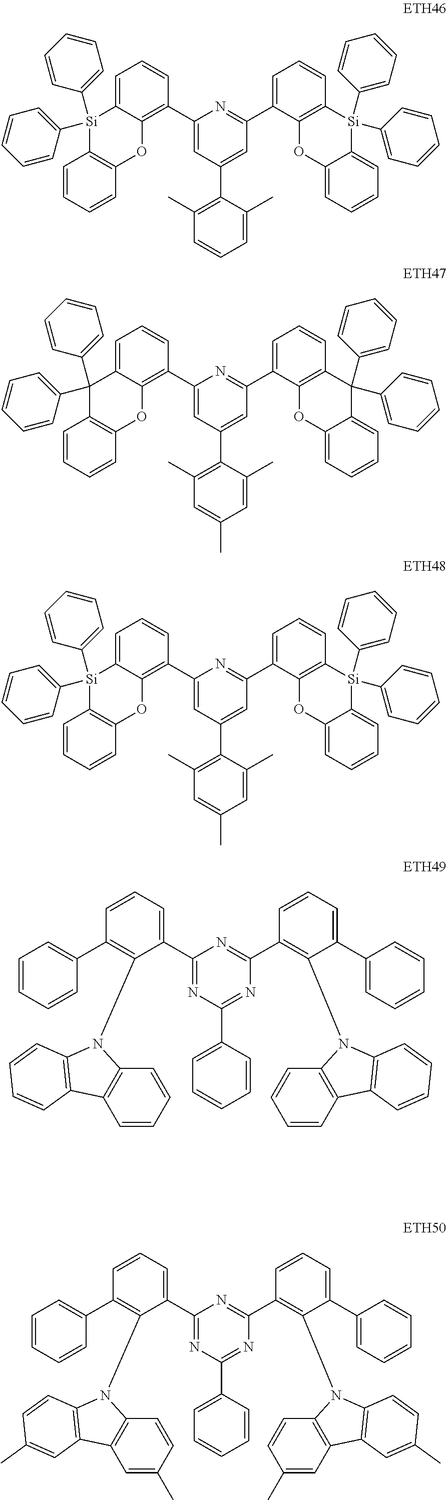

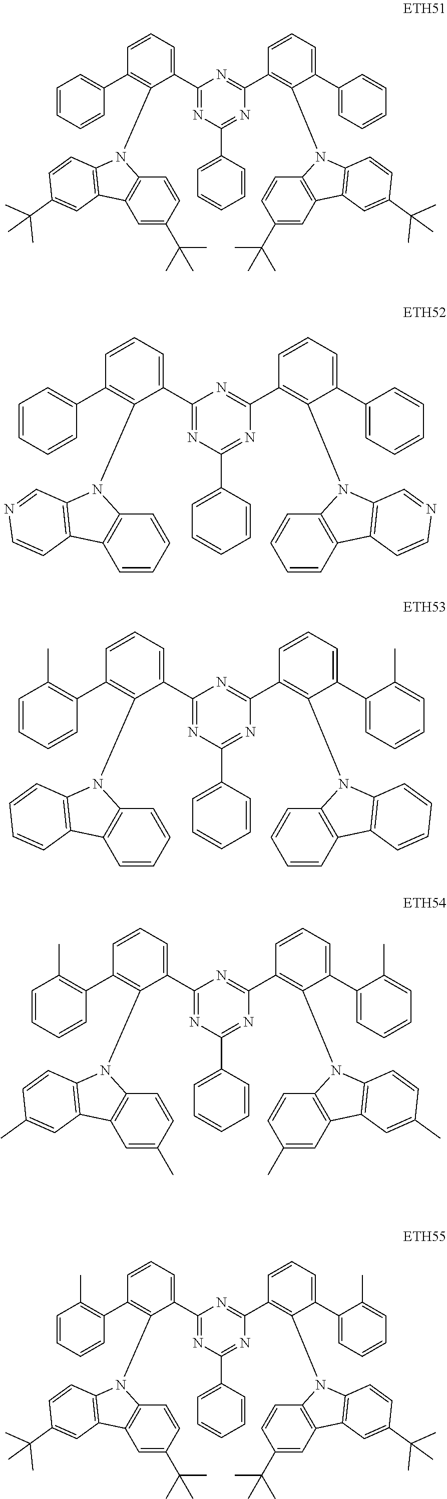

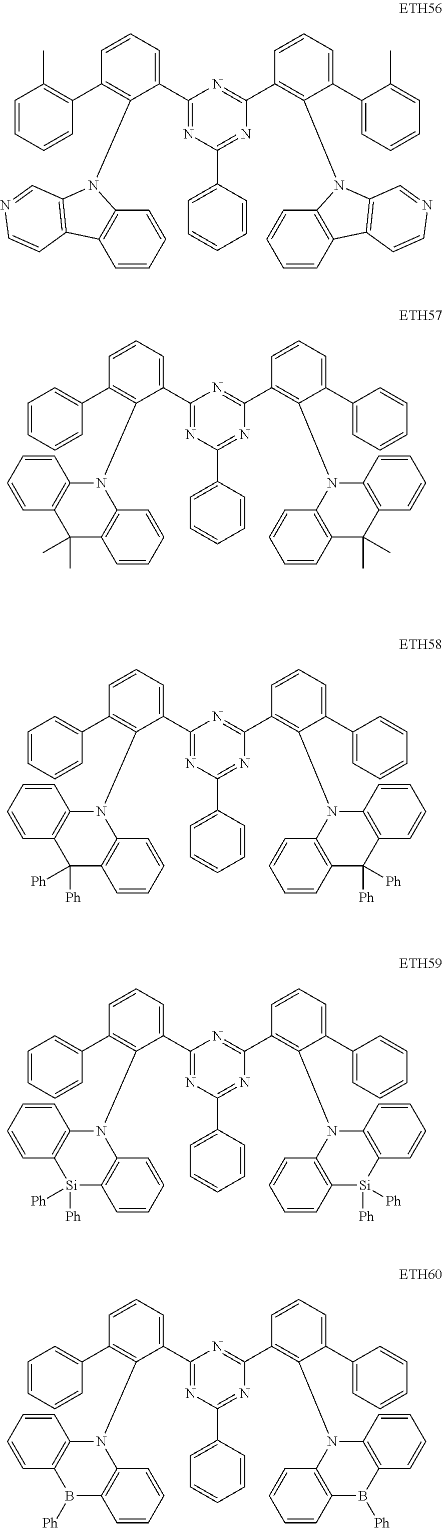

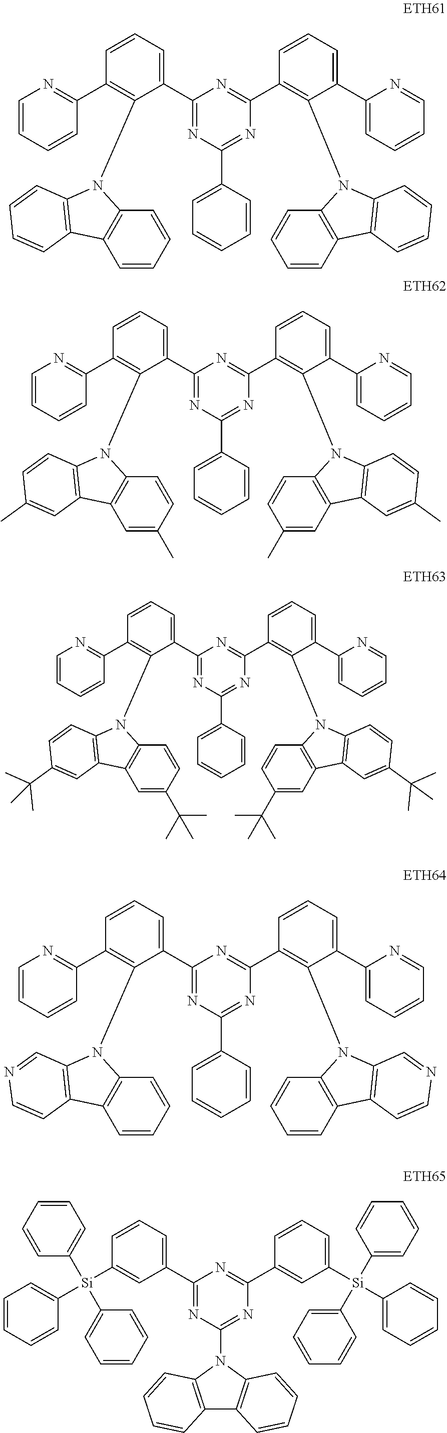

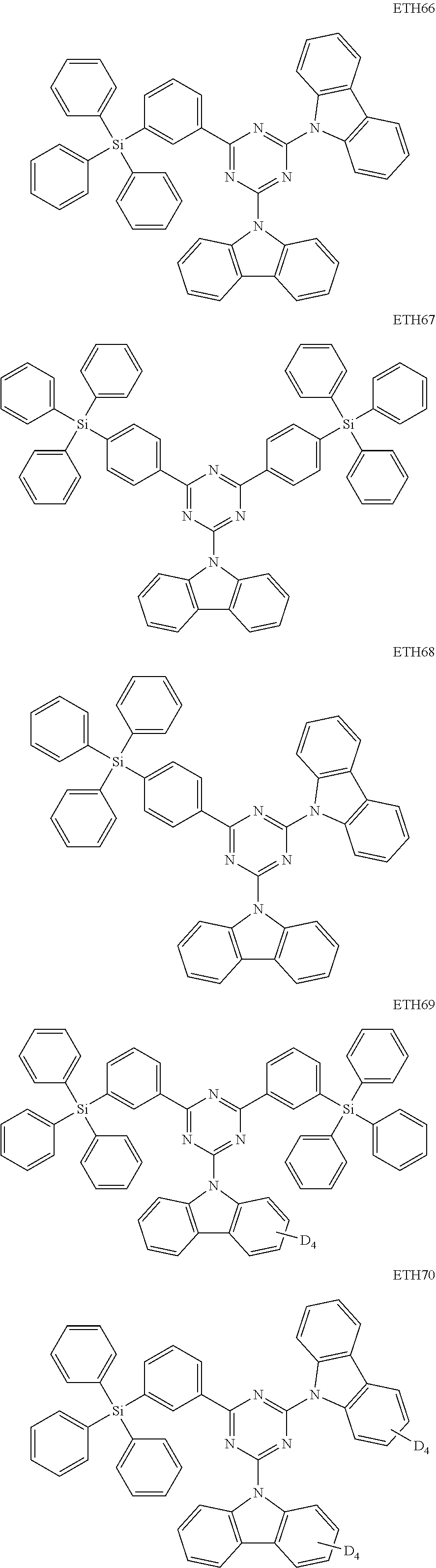

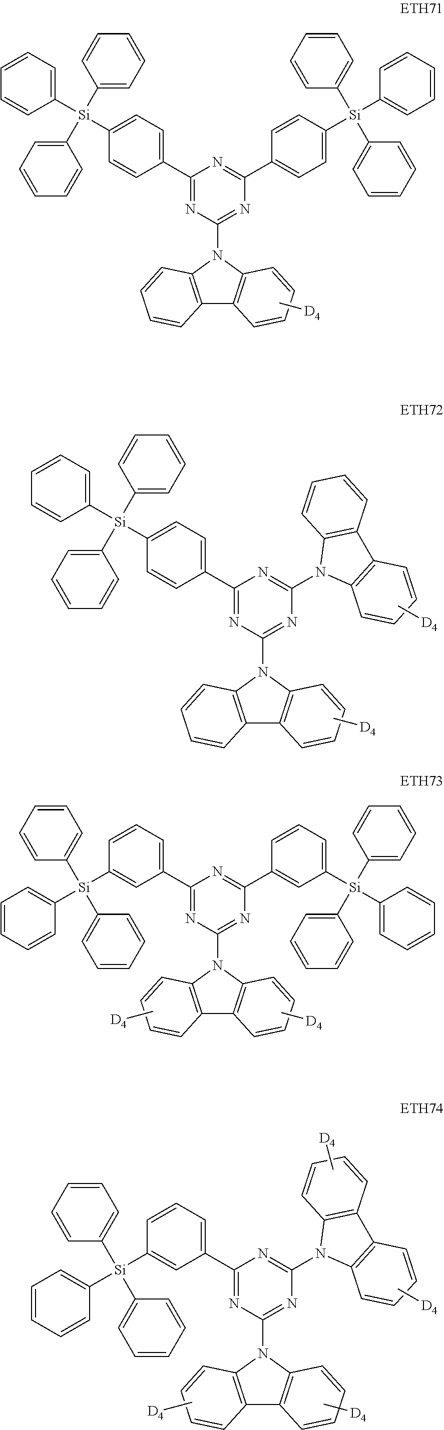

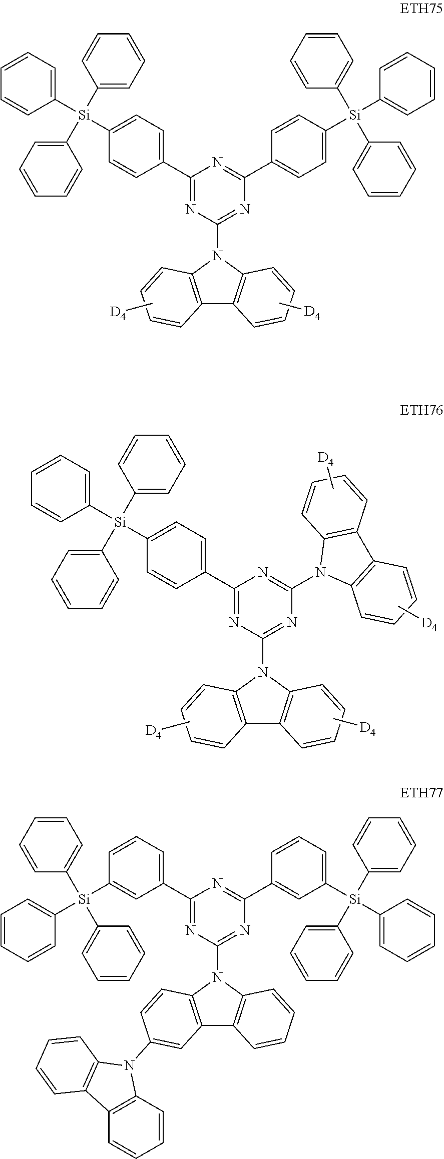

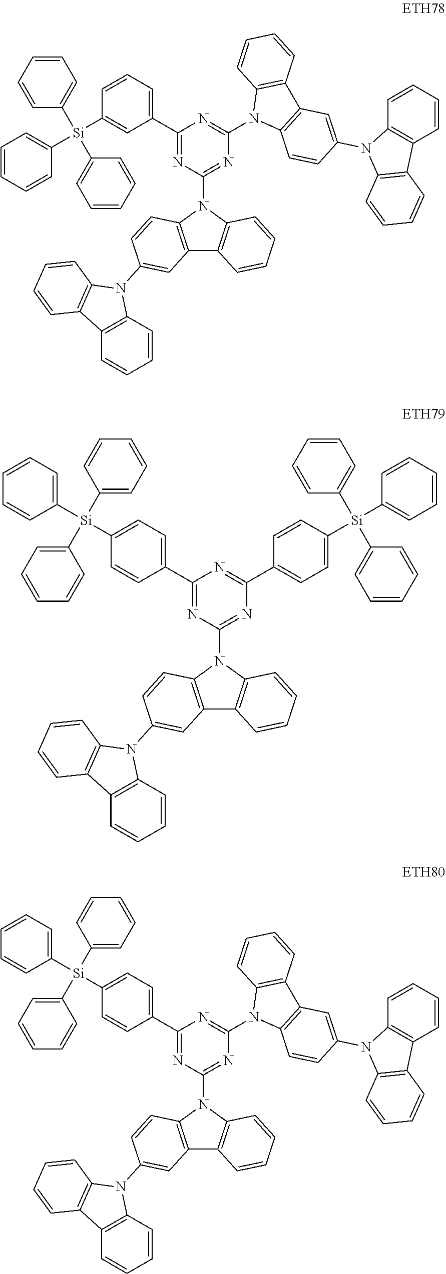 [SMILES: c1ccc([Si](c2ccccc2)(c2ccccc2)c2cccc(-c3nc(-n4c5ccccc5c5cc(-n6c7ccccc7c7ccccc76)ccc54)nc(-n4c5ccccc5c5cc(-n6c7ccccc7c7ccccc76)ccc54)n3)c2)cc1 ; c1ccc([Si](c2ccccc2)(c2ccccc2)c2ccc(-c3nc(-c4ccc([Si](c5ccccc5)(c5ccccc5)c5ccccc5)cc4)nc(-n4c5ccccc5c5cc(-n6c7ccccc7c7ccccc76)ccc54)n3)cc2)cc1 ; c1ccc([Si](c2ccccc2)(c2ccccc2)c2ccc(-c3nc(-n4c5ccccc5c5cc(-n6c7ccccc7c7ccccc76)ccc54)nc(-n4c5ccccc5c5cc(-n6c7ccccc7c7ccccc76)ccc54)n3)cc2)cc1]

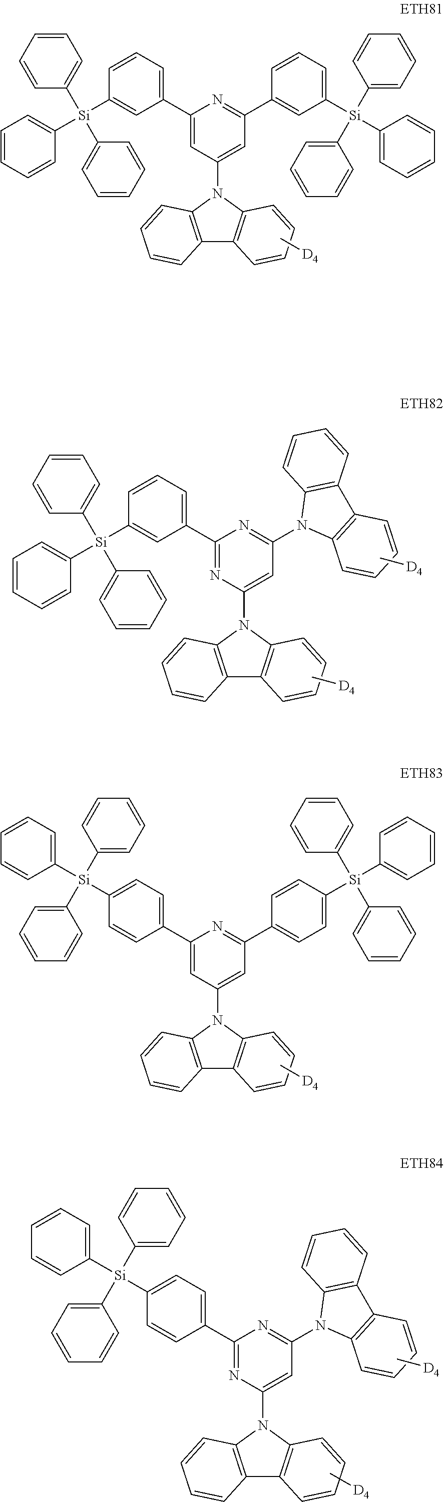

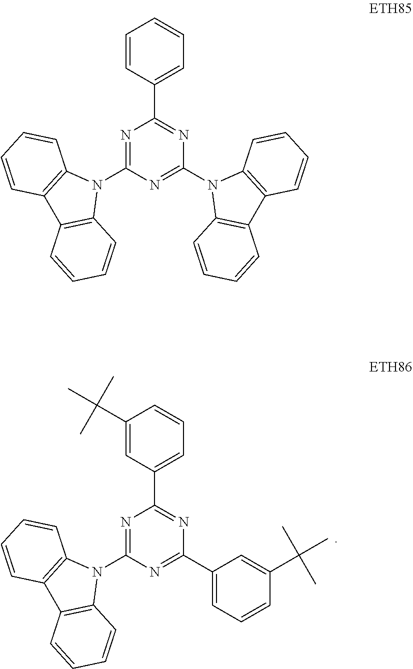 [SMILES: c1ccc(-c2nc(-n3c4ccccc4c4ccccc43)nc(-n3c4ccccc4c4ccccc43)n2)cc1 ; CC(C)(C)c1cccc(-c2nc(-c3cccc(C(C)(C)C)c3)nc(-n3c4ccccc4c4ccccc43)n2)c1]

In embodiment compounds presented in Compound Group 3, “D” may refer to a deuterium atom, and “Ph” may refer to an unsubstituted phenyl group.

The emission layer EML may include the second compound and the third compound, and the second compound and the third compound may form an exciplex. In the emission layer EML, an exciplex may be formed by the hole transport host and the electron transport host. In this case, a triplet energy of the exciplex formed by the hole transporting host and the electron transporting host may correspond to the difference between a lowest unoccupied molecular orbital (LUMO) energy level of the electron transporting host and a highest occupied molecular orbital (HOMO) energy level of the hole transporting host.

For example, the absolute value of the triplet energy (T1) of the exciplex formed by the hole transporting host and the electron transporting host may be about 2.4 eV to about 3.0 eV. In some embodiments, the triplet energy of the exciplex may be a value smaller than an energy gap of each host material. The exciplex may have a triplet energy of about 3.0 eV or less that is an energy gap between the hole transporting host and the electron transporting host.

In an embodiment, the emission layer EML may include a fourth compound in addition to the first compound to the third compound. The fourth compound may be utilized as a phosphorescent sensitizer of the emission layer EML. The energy may be transferred from the fourth compound to the first compound, thereby emitting light.

For example, the emission layer EML may include, as the fourth compound, an organometallic complex containing platinum (Pt) as a central metal atom and ligands linked to the central metal atom. The emission layer EML in the light emitting device ED of an embodiment may include, as the fourth compound, a compound represented by Formula D-1:

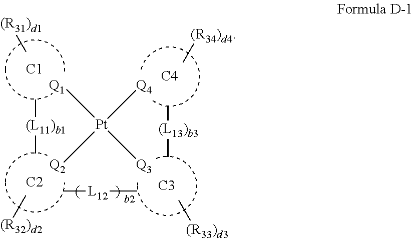

In Formula D-1, Q₁ to Q₄ may each independently be C or N.

In Formula D-1, C1 to C4 may each independently be a substituted or unsubstituted hydrocarbon ring having 5 to 30 ring-forming carbon atoms, or a substituted or unsubstituted heterocycle having 2 to 30 ring-forming carbon atoms.

In Formula D-1, Li to L₁₃ may each independently be a direct linkage,

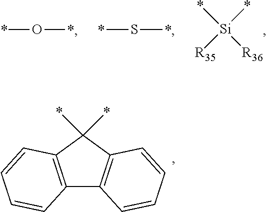

a substituted or unsubstituted divalent alkyl group having 1 to 20 carbon atoms, a substituted or unsubstituted arylene group having 6 to 30 ring-forming carbon atoms, or a substituted or unsubstituted heteroarylene group having 2 to 30 ring-forming carbon atoms. In L₂1 to L₂₃, “—*” refers to a part linked to C1 to C4.

In Formula D-1, b1 to b3 may each independently be 0 or 1. When b1 is 0, C1 and C2 may not be linked to each other. When b2 is 0, C2 and C3 may not be linked to each other. When b3 is 0, C3 and C4 may not be linked to each other.

In Formula D-1, R₃₁ to R₃₆ may each independently be a hydrogen atom, a deuterium atom, a halogen atom, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group having 1 to 20 carbon atoms, a substituted or unsubstituted alkenyl group having 2 to 20 carbon atoms, a substituted or unsubstituted aryl group having 6 to 60 ring-forming carbon atoms, or a substituted or unsubstituted heteroaryl group having 2 to 60 ring-forming carbon atoms. In some embodiments, each of R₃₁ to R₃₆ may be bonded to an adjacent group to form a ring. R₃₁ to R₃₆ may each independently be a substituted or unsubstituted methyl group, or a substituted or unsubstituted t-butyl group.

In Formula D-1, d1 to d4 may each independently be an integer of 0 to 4. In Formula D-1, when any or each of d1 to d4 is 0, the fused polycyclic compound of an embodiment may not be substituted with any or each of R₃₁ to R₃₄. The case where any or each of d1 to d4 is 4 and any or each of R₃₁'s to R₃₄′ are hydrogen atoms may be the same as the case where any or each of d1 to d4 is 0. When any or each of d1 to d4 is an integer of 2 or more, a plurality of R₃₁'s to R₃₄'s may each be the same or at least one from among the plurality of R₃₁'s to R₃₄'s may be different from the others.

In Formula D-1, C1 to C4 may each independently be a substituted or unsubstituted hydrocarbon ring or a substituted or unsubstituted heterocycle represented by any one from among C-1 to C-4:

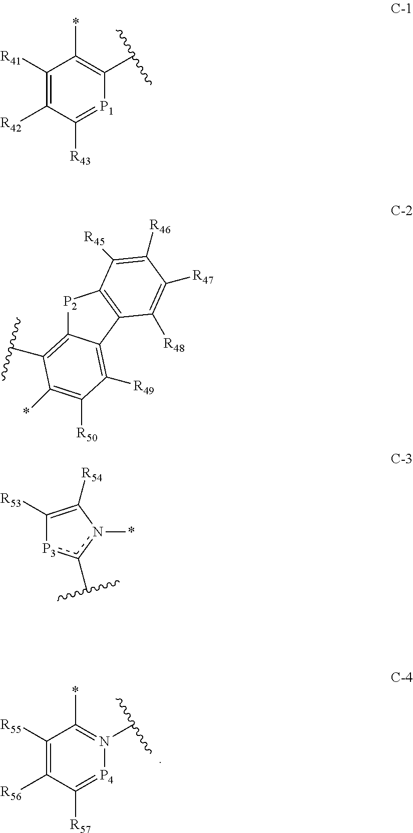

In C-1 to C-4, P₁ may be C—* or CR₄₄, P₂ may be N—* or NR₅₁, P₃ may be N—* or NR₅₂, and P₄ may be C—* or CR₅₈. R₄₁ to R₅₈ may each independently be a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and/or bonded to an adjacent group to form a ring.

In some embodiments, in C-1 to C-4,

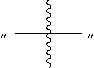

corresponds to a part linked to Pt that is a central metal atom, and “—*” corresponds to a part linked to a neighboring cyclic group (C1 to C4) or a linker (L₁₁ to L₁₃).

The emission layer EML of an embodiment may include the first compound, which is a fused polycyclic compound, and at least one of the second to fourth compounds. For example, the emission layer EML may include the first compound, the second compound, and the third compound. In the emission layer EML, the second compound and the third compound may form an exciplex, and the energy may be transferred from the exciplex to the first compound, thereby emitting light.

In some embodiments, the emission layer EML may include the first compound, the second compound, the third compound, and the fourth compound. In the emission layer EML, the second compound and the third compound may form an exciplex, and the energy may be transferred from the exciplex to the fourth compound and the first compound, thereby emitting light. In an embodiment, the fourth compound may be a sensitizer. The fourth compound included in the emission layer EML in the light emitting device ED of an embodiment may serve as a sensitizer to deliver energy from the host to the first compound that is a light emitting dopant. For example, the fourth compound serving as an auxiliary dopant accelerates energy delivery to the first compound that is a light emitting dopant, thereby increasing the emission ratio of the first compound. Therefore, the emission layer EML of an embodiment may improve luminous efficiency. In some embodiments, when the energy delivery to the first compound is increased, an exciton formed in the emission layer EML is not accumulated inside the emission layer EML and emits light rapidly, and thus deterioration of the element may be reduced. Therefore, the service life of the light emitting device ED of an embodiment may increase.

The light emitting device ED of an embodiment may include all of the first compound, the second compound, the third compound, and the fourth compound, and the emission layer EML may include the combination of two host materials and two dopant materials. In the light emitting device ED of an embodiment, the emission layer EML may concurrently (e.g., simultaneously) include the second compound and the third compound, which are two different hosts, the first compound that emits a delayed fluorescence, and the fourth compound including an organometallic complex, thereby exhibiting excellent or suitable luminous efficiency characteristics.

In an embodiment, the fourth compound represented by Formula D-1 may represented at least one from among the compounds represented by Compound Group 4. The emission layer EML may include at least one from among the compounds represented by Compound Group 4 as a sensitizer material.

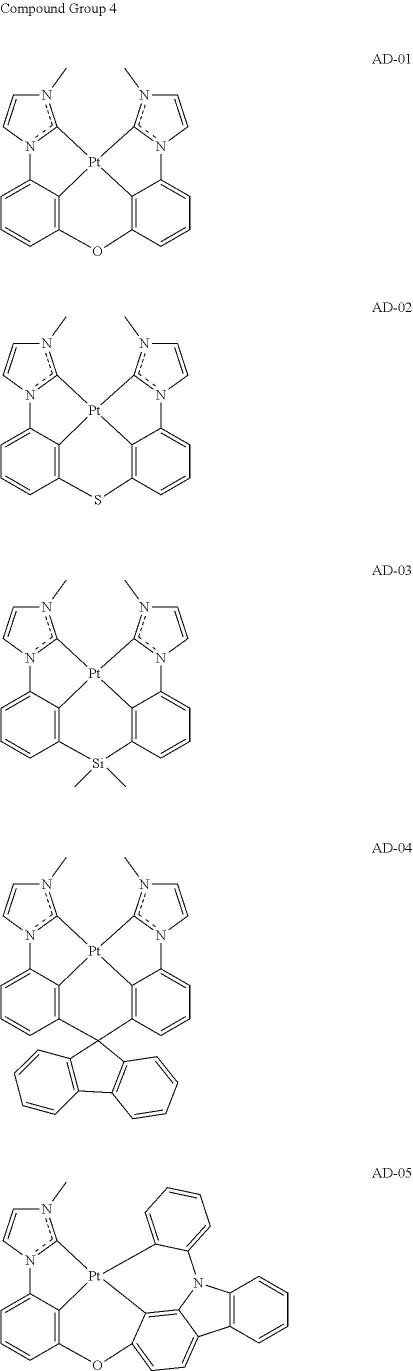

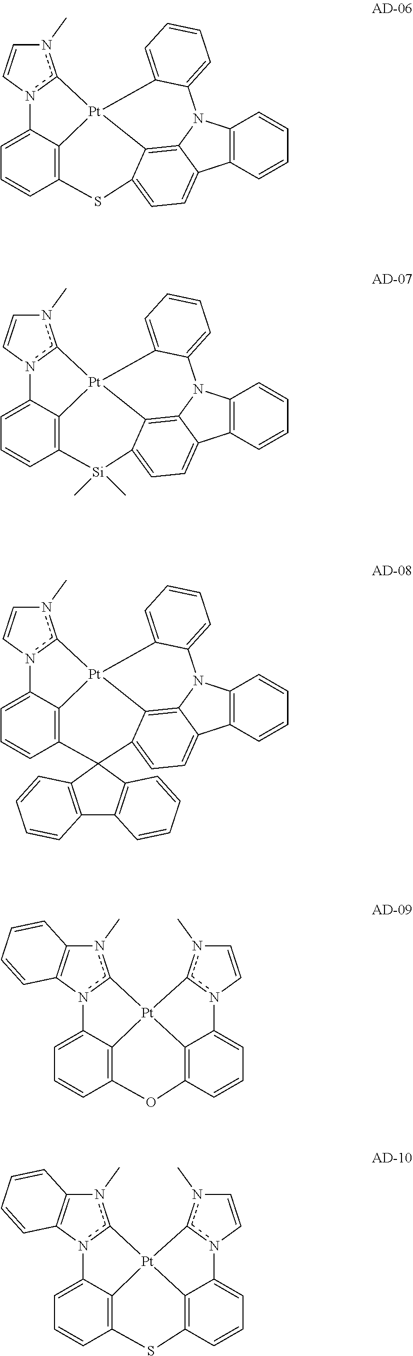

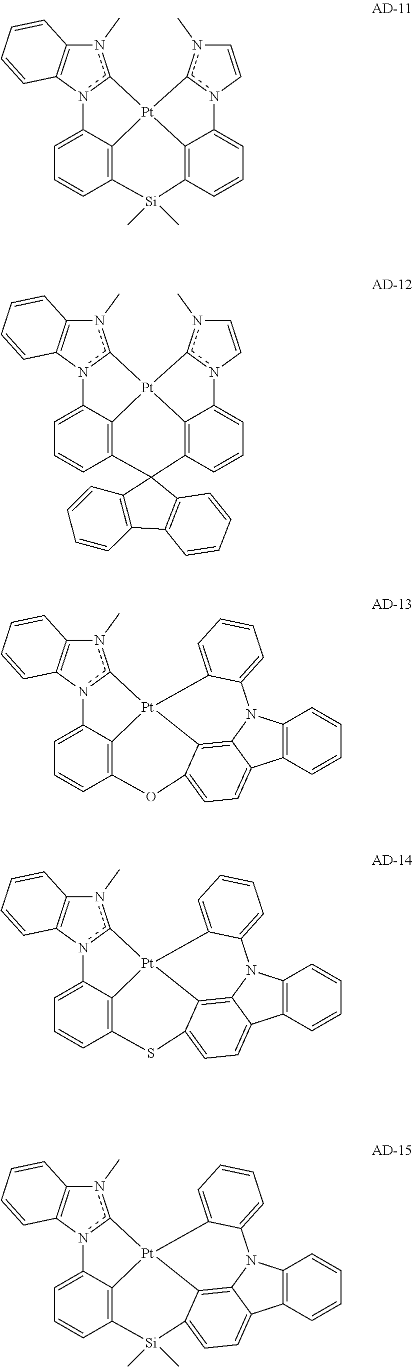

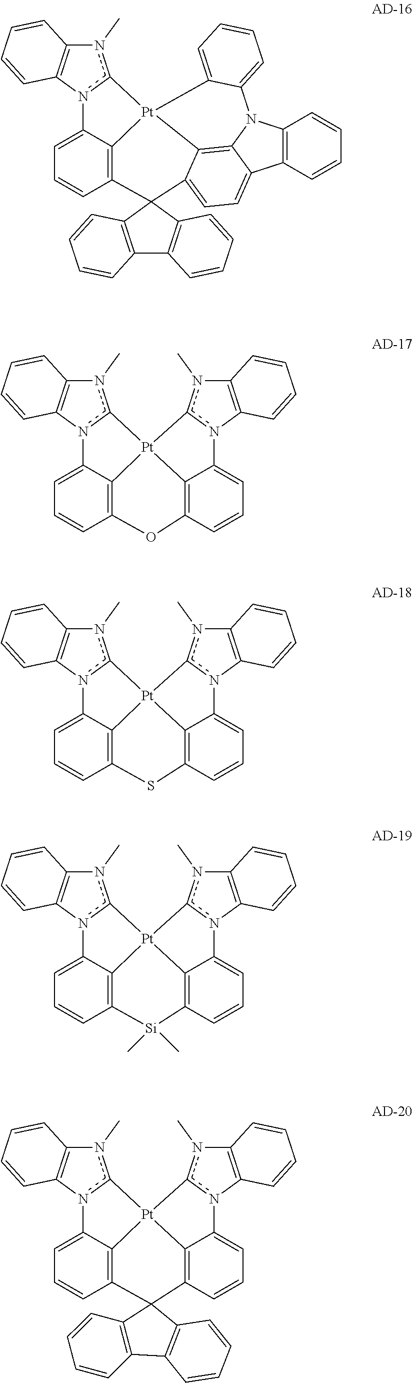

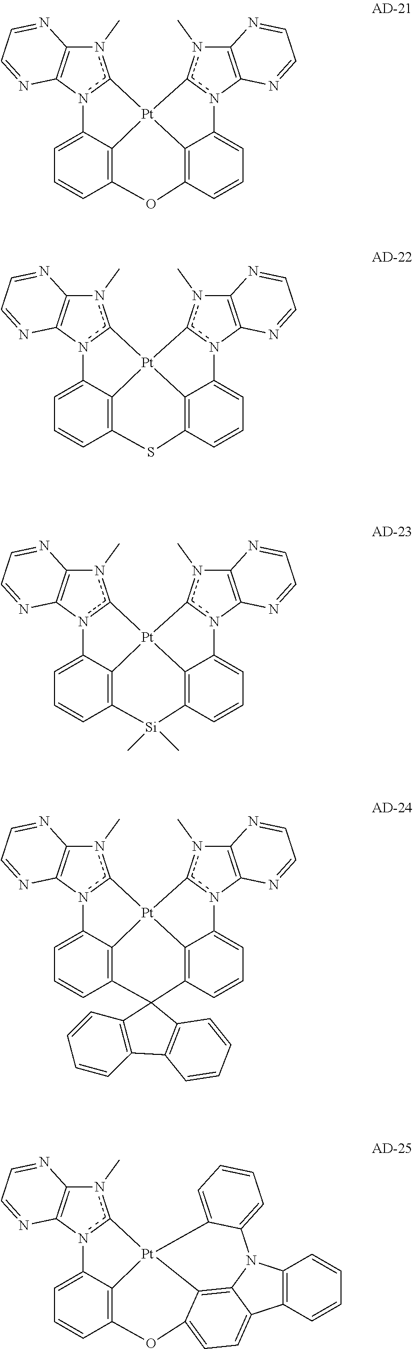

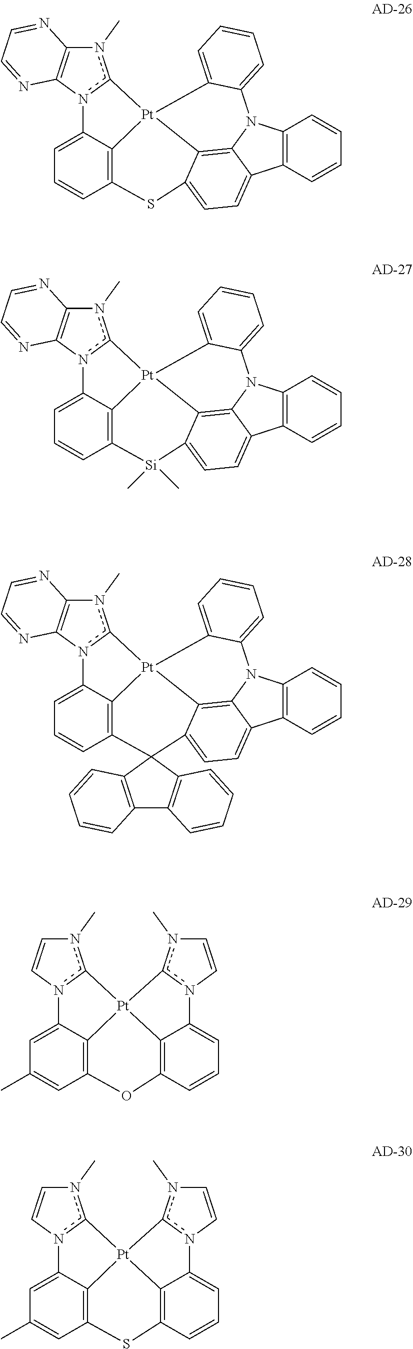

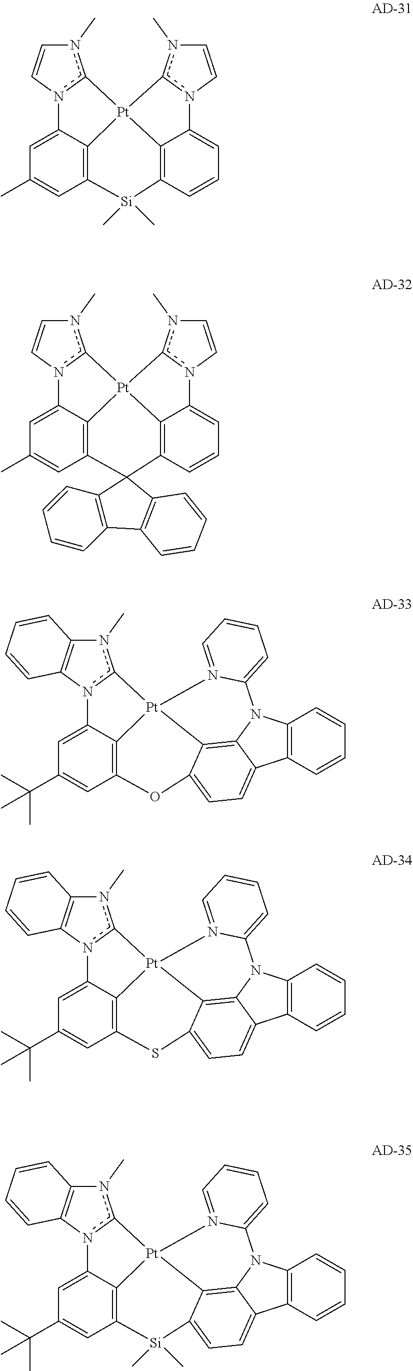

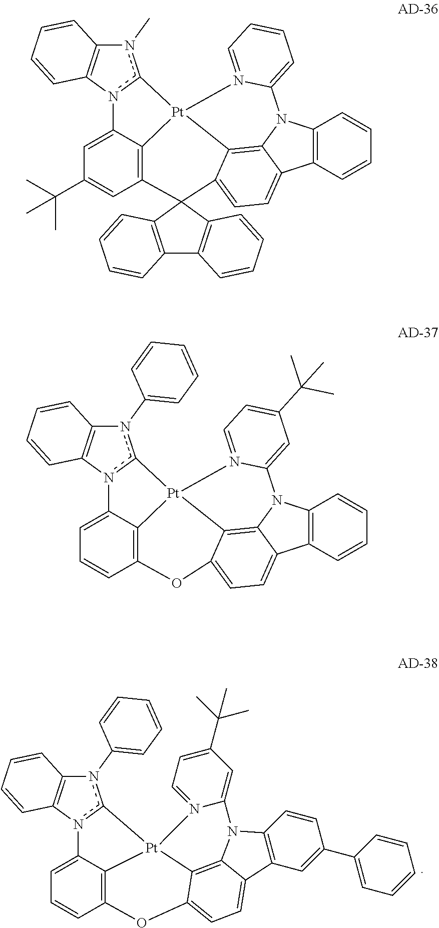

In some embodiments, the light emitting device ED of an embodiment may include a plurality of emission layers. The plurality of emission layers may be sequentially stacked and provided, and for example, the light emitting device ED including the plurality of emission layers may be to emit white light. The light emitting device including the plurality of emission layers may be a light emitting device having a tandem structure. When the light emitting device ED includes a plurality of emission layers, at least one emission layer EML may include the first compound represented by Formula 1 of an embodiment. In some embodiments, when the light emitting device ED includes the plurality of emission layers, at least one emission layer EML may include all of the first compound, the second compound, the third compound, and the fourth compound as described above.

When the emission layer EML in the light emitting device ED of an embodiment includes all of the first compound, the second compound, the third compound, and the fourth compound, with respect to the total weight of the first compound, the second compound, the third compound, and the fourth compound, the content (e.g., amount) of the first compound may be about 0.1 wt % to about 5 wt %. However, an embodiment of the present disclosure is not limited thereto. When the content (e.g., amount) of the first compound satisfy the above-described proportion, the energy transfer from the second compound and the third compound to the first compound may increase, and thus the luminous efficiency and device service life may increase.

The contents of the second compound and the third compound in the emission layer EML may be the rest (e.g., any remainder thereof) excluding the weights of the first compound and the fourth compound as described above. For example, the contents of the second compound and the third compound in the emission layer EML may be about 75 wt % to about 95 wt % with respect to the total weight of the first compound, the second compound, the third compound, and the fourth compound.

In the total weight of the second compound and the third compound, the weight ratio between the second compound and the third compound may be about 3:7 to about 7:3.

When the contents of the second compound and the third compound satisfy the above-described ratio, a charge balance characteristic in the emission layer EML may be improved, and thus the luminous efficiency and device service life may increase. When the contents of the second compound and the third compound deviate from the above-described ratio range, a charge balance in the emission layer EML may be broken, and thus the luminous efficiency may be reduced and the device may be easily deteriorated.

The content (e.g., amount) of the fourth compound in the emission layer EML may be about 4 wt % to about 20 wt % with respect to the total weight of the first compound, the second compound, the third compound, and the fourth compound. However, the embodiment of the present disclosure is not limited thereto. When the content (e.g., amount) of the fourth compound satisfies the above-described content (e.g., amount), the energy delivery from the host to the first compound which is a light emitting dopant may be increased, thereby a luminous ratio may be improved, and thus the luminous efficiency of the emission layer EML may be improved.

When the first compound, the second compound, the third compound, and the fourth compound included in the emission layer EML satisfy the above-described content (e.g., amount) ranges (e.g., ratio ranges), excellent or suitable luminous efficiency and long service life may be achieved.

In the light emitting device ED of an embodiment, the emission layer EML may include an anthracene derivative, a pyrene derivative, a fluoranthene derivative, a chrysene derivative, a dehydrobenzanthracene derivative, and/or a triphenylene derivative. For example, the emission layer EML may include the anthracene derivative and/or the pyrene derivative.

In each light emitting device ED of embodiments illustrated in FIGS. 3 to 6, the emission layer EML may further include a suitable host and dopant besides the above-described host and dopant, and for example the emission layer EML may include a compound represented by Formula E-1. The compound represented by Formula E-1 may be utilized as a fluorescent host material.

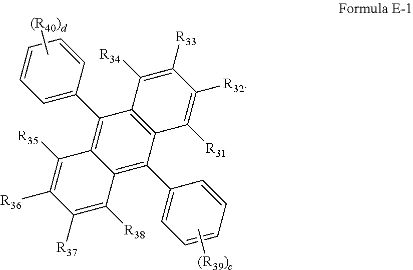

In Formula E-1, R₃₁ to R₄₀ may each independently be a hydrogen atom, a deuterium atom, a halogen atom, a substituted or unsubstituted silyl group, a substituted or unsubstituted thio group, a substituted or unsubstituted oxy group, a substituted or unsubstituted alkyl group having 1 to 10 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, and/or bonded to an adjacent group to form a ring. In some embodiments, R₃₁ to R₄₀ may be bonded to an adjacent group to form a saturated hydrocarbon ring or an unsaturated hydrocarbon ring, a saturated heterocycle, or an unsaturated heterocycle.

In Formula E-1, c and d may each independently be an integer of 0 to 5.

Formula E-1 may be represented by any one from among Compound E1 to Compound E19:

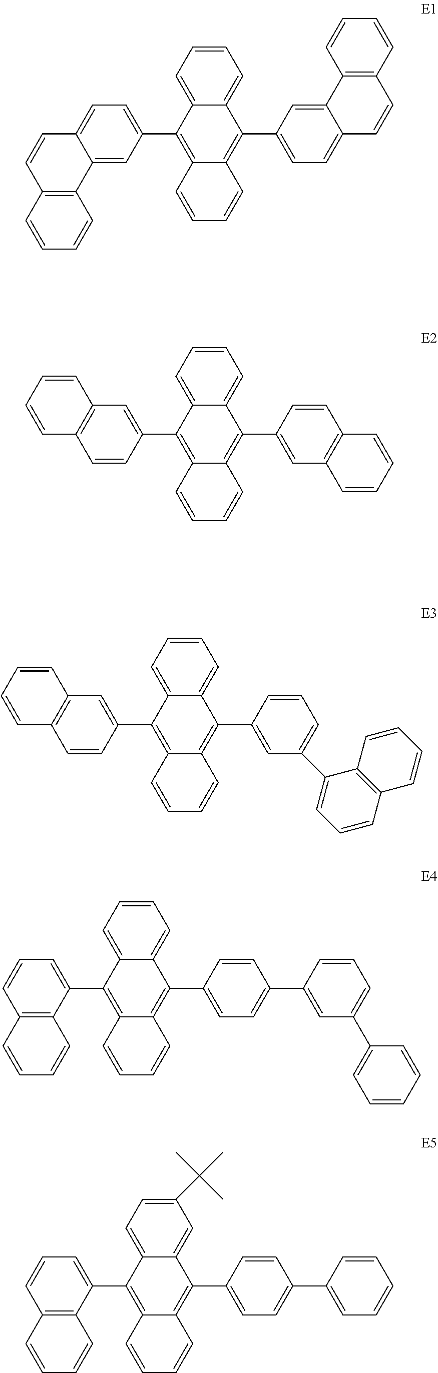

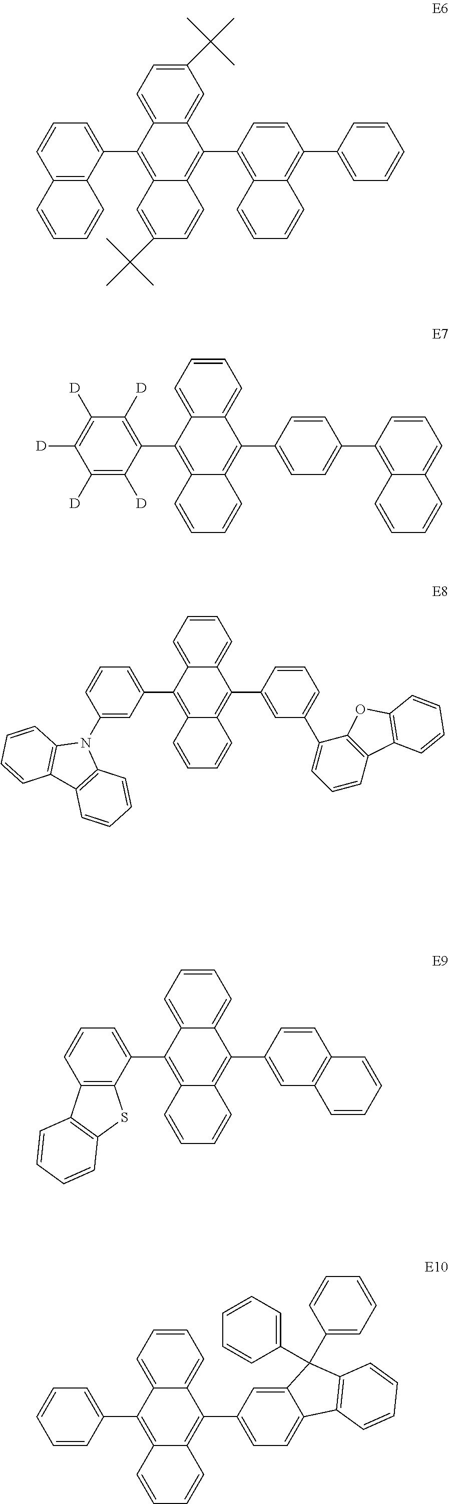

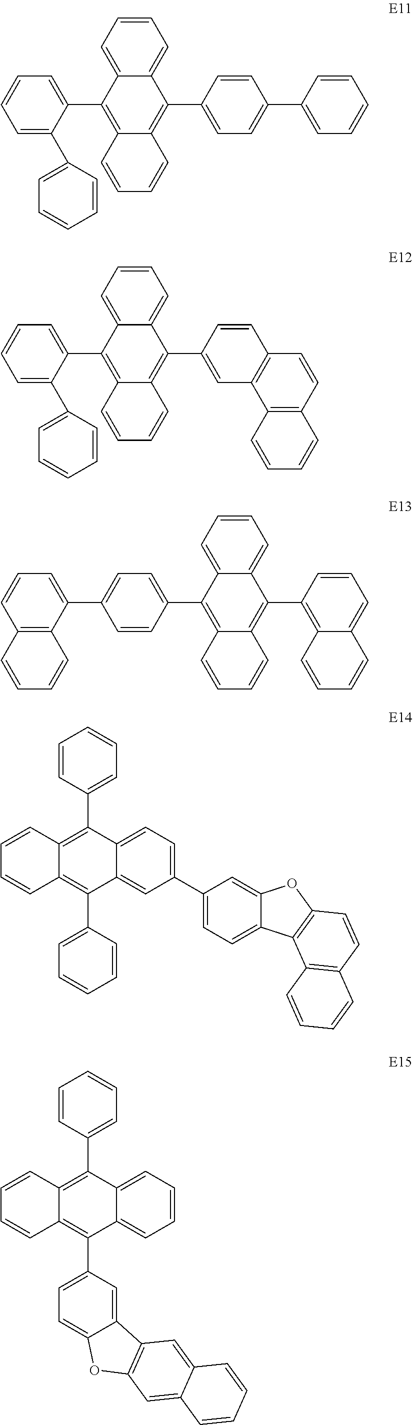

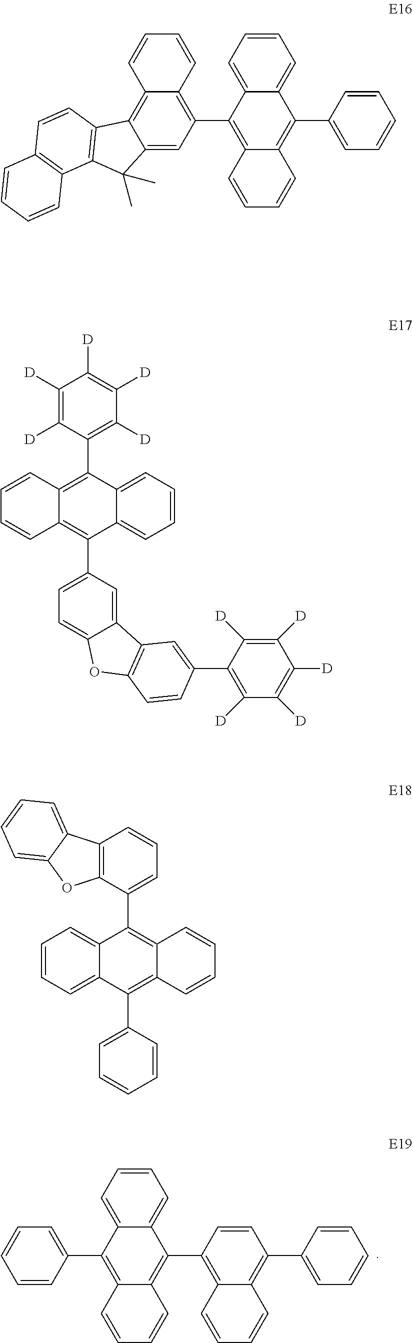

In an embodiment, the emission layer EML may include a compound represented by Formula E-2a or Formula E-2b. The compound represented by Formula E-2a or Formula E-2b may be utilized as a phosphorescent host material.

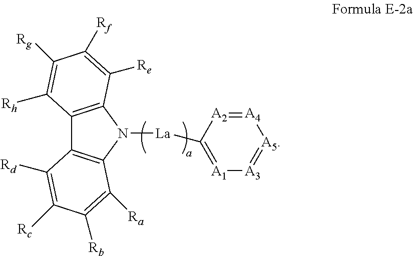

In Formula E-2a, a may be an integer of 0 to 10, and La may be a direct linkage, a substituted or unsubstituted arylene group having 6 to 30 ring-forming carbon atoms, or a substituted or unsubstituted heteroarylene group having 2 to 30 ring-forming carbon atoms. In some embodiments, when a is an integer of 2 or more, a plurality of La's may each independently be a substituted or unsubstituted arylene group having 6 to 30 ring-forming carbon atoms, or a substituted or unsubstituted heteroarylene group having 2 to 30 ring-forming carbon atoms.

In some embodiments, in Formula E-2a, A₁ to A₅ may each independently be N or CRᵢ. Rₐ to Rᵢ may each independently be a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, and/or bonded to an adjacent group to form a ring. In an embodiment, Rₐ to Rᵢ may be bonded to an adjacent group to form a hydrocarbon ring or a heterocycle containing N, O, S, etc. as a ring-forming atom.

In some embodiments, in Formula E-2a, two or three selected from among A₁ to A₅ may be N, and any remainder thereof may be CRᵢ.

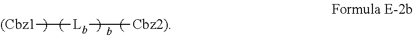

In Formula E-2b, Cbz1 and Cbz2 may each independently be an unsubstituted carbazole group, or a carbazole group substituted with an aryl group having 6 to 30 ring-forming carbon atoms. L_{b} is a direct linkage, a substituted or unsubstituted arylene group having 6 to 30 ring-forming carbon atoms, or a substituted or unsubstituted heteroarylene group having 2 to 30 ring-forming carbon atoms. In some embodiments, b is an integer of 0 to 10, and when b is an integer of 2 or more, a plurality of L_{b}'s may each independently be a substituted or unsubstituted arylene group having 6 to 30 ring-forming carbon atoms, or a substituted or unsubstituted heteroarylene group having 2 to 30 ring-forming carbon atoms.

The compound represented by Formula E-2a or Formula E-2b may be represented by any one from among the compounds of Compound Group E-2. However, the compounds listed in Compound Group E-2 are only examples, and the compound represented by Formula E-2a or Formula E-2b is not limited to those represented in Compound Group E-2.

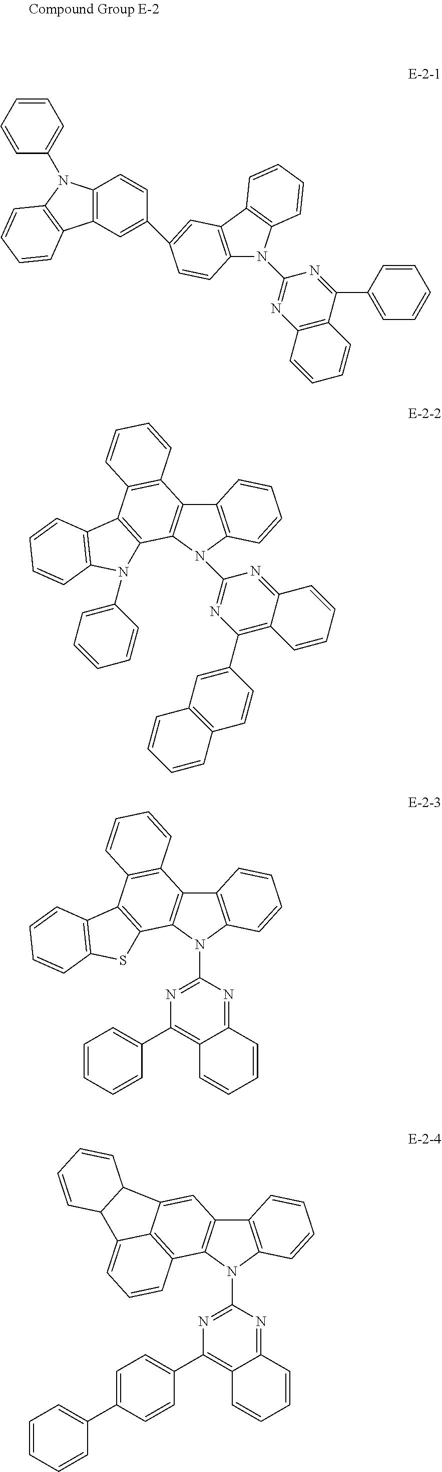

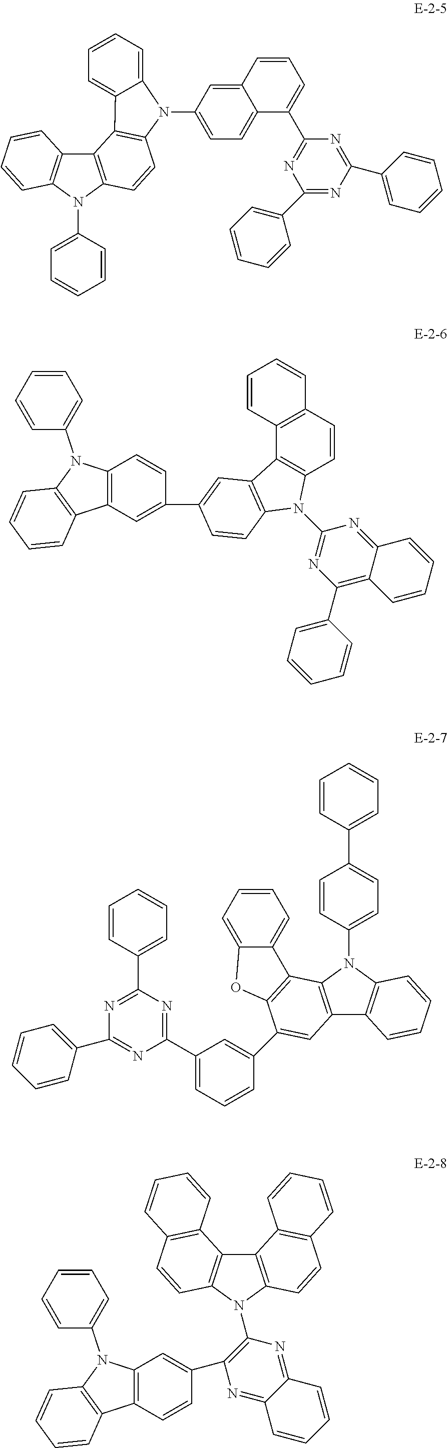 [SMILES: c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1 ; c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-n2c3ccccc3c3ccc(-c4nc5ccccc5nc4-n4c5ccc6ccccc6c5c5c6ccccc6ccc54)cc32)cc1]

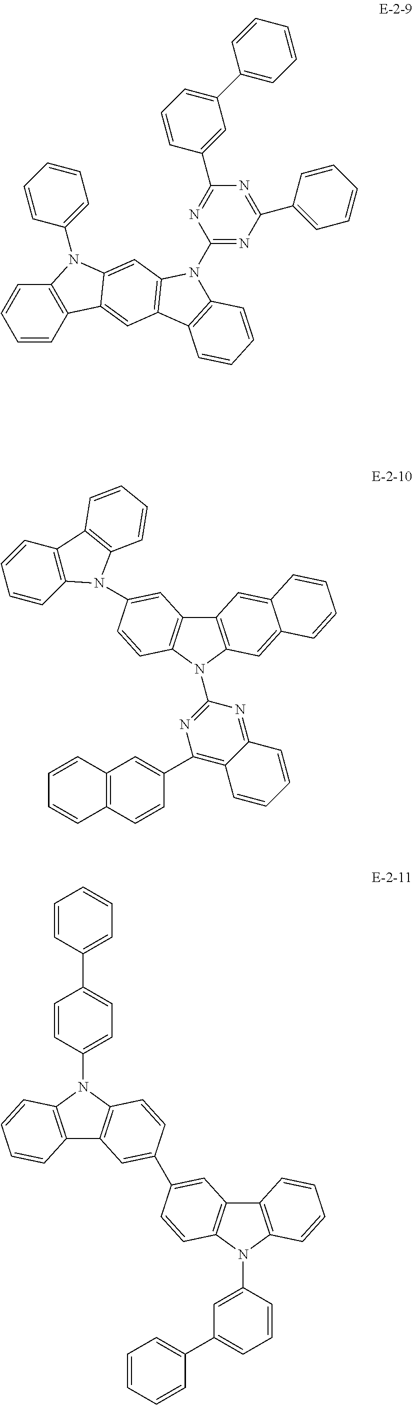 [SMILES: c1ccc(-c2cccc(-c3nc(-c4ccccc4)nc(-n4c5ccccc5c5cc6c7ccccc7n(-c7ccccc7)c6cc54)n3)c2)cc1 ; c1ccc2cc(-c3nc(-n4c5ccc(-n6c7ccccc7c7ccccc76)cc5c5cc6ccccc6cc54)nc4ccccc34)ccc2c1 ; c1ccc(-c2ccc(-n3c4ccccc4c4cc(-c5ccc6c(c5)c5ccccc5n6-c5cccc(-c6ccccc6)c5)ccc43)cc2)cc1]

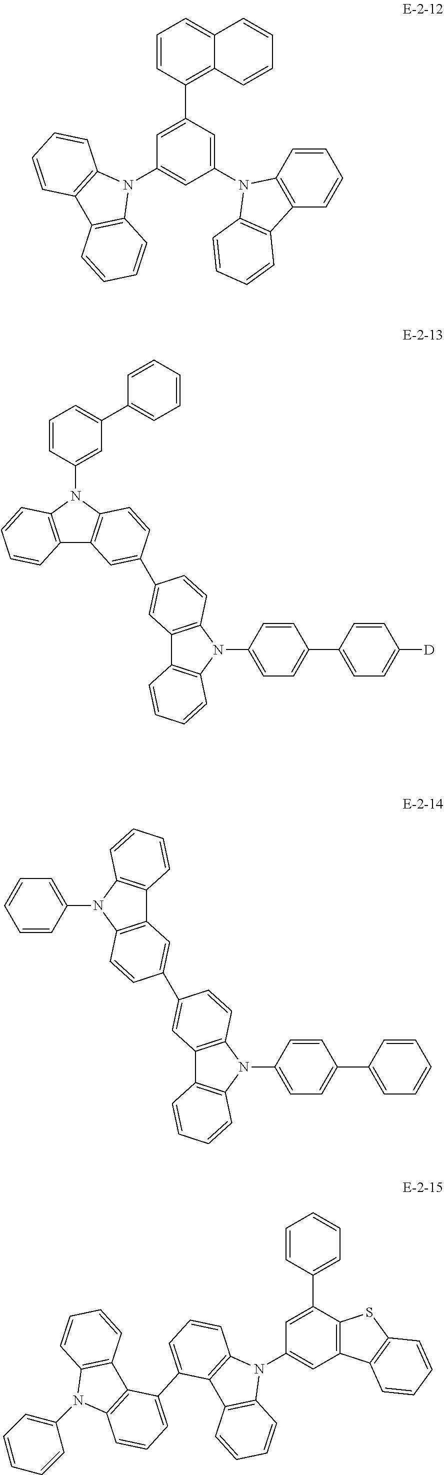 [SMILES: c1ccc2c(-c3cc(-n4c5ccccc5c5ccccc54)cc(-n4c5ccccc5c5ccccc54)c3)cccc2c1 ; [2H]c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1]

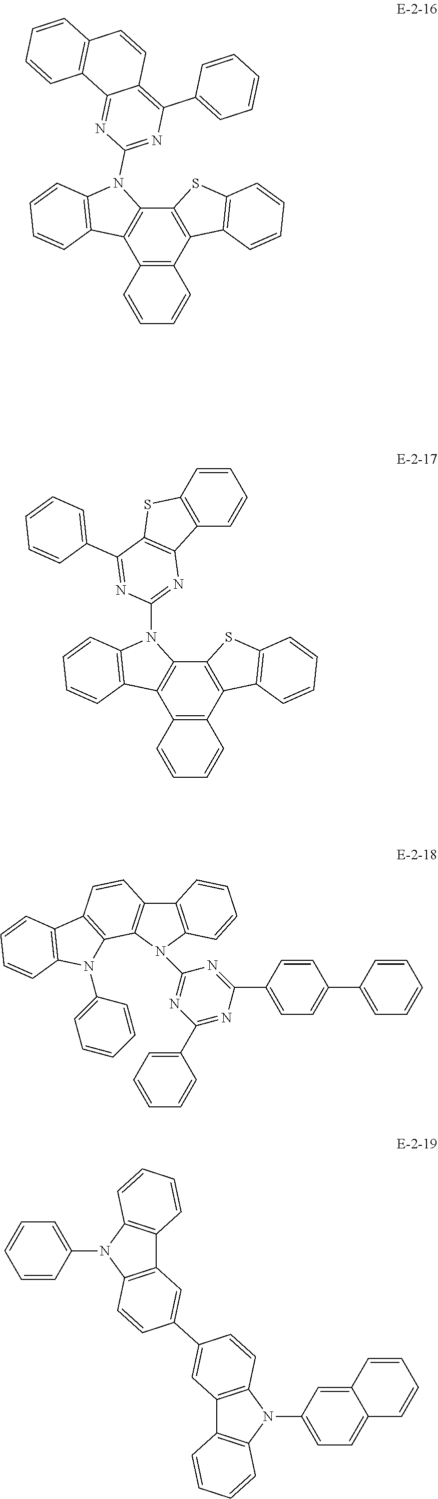 [SMILES: c1ccc(-c2nc(-n3c4ccccc4c4c5ccccc5c5c6ccccc6sc5c43)nc3c2ccc2ccccc23)cc1 ; c1ccc(-c2nc(-n3c4ccccc4c4c5ccccc5c5c6ccccc6sc5c43)nc3c2sc2ccccc23)cc1 ; c1ccc(-c2ccc(-c3nc(-c4ccccc4)nc(-n4c5ccccc5c5ccc6c7ccccc7n(-c7ccccc7)c6c54)n3)cc2)cc1 ; c1ccc(-n2c3ccccc3c3cc(-c4ccc5c(c4)c4ccccc4n5-c4ccc5ccccc5c4)ccc32)cc1]

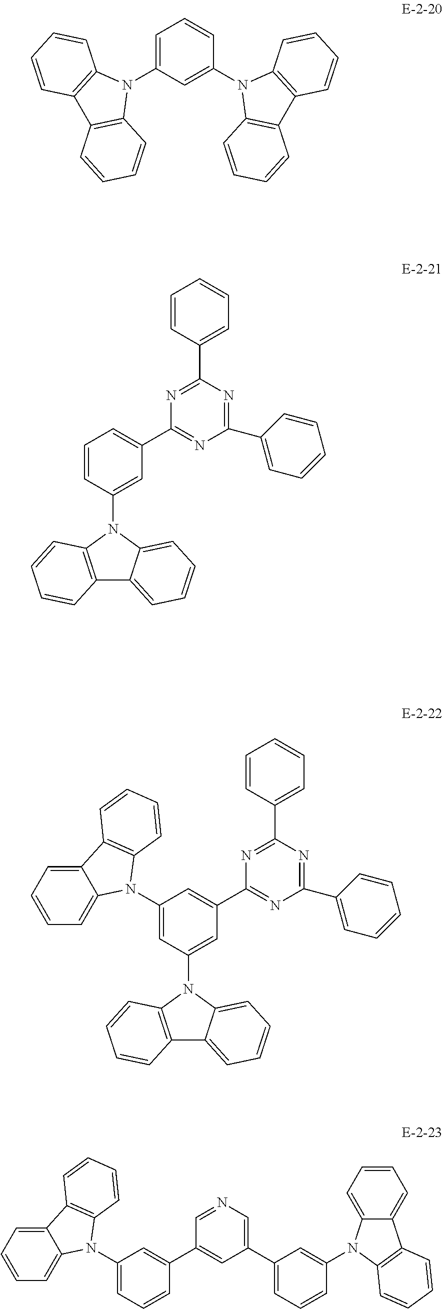 [SMILES: c1cc(-n2c3ccccc3c3ccccc32)cc(-n2c3ccccc3c3ccccc32)c1 ; c1ccc(-c2nc(-c3ccccc3)nc(-c3cccc(-n4c5ccccc5c5ccccc54)c3)n2)cc1 ; c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1]

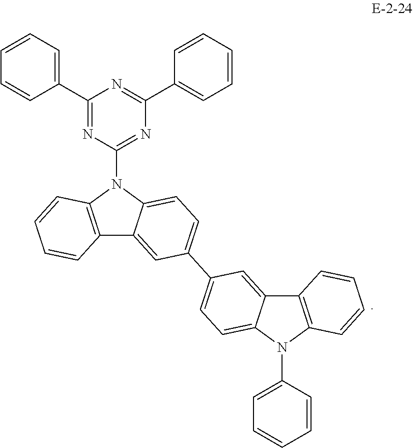 [SMILES: c1ccc(-c2nc(-c3ccccc3)nc(-n3c4ccccc4c4cc(-c5ccc6c(c5)c5ccccc5n6-c5ccccc5)ccc43)n2)cc1]

The emission layer EML may further include a general material suitable in the art as a host material. For example, the emission layer EML may include, as a host material, at least one of bis(4-(9H-carbazol-9-yl)phenyl)diphenylsilane (BCPDS),
(4-(1-(4-(diphenylamino)phenyl)cyclohexyl)phenyl)diphenyl-phosphine oxide (POPCPA), bis[2-(diphenylphosphino)phenyl]ether oxide (DPEPO),
4,4′-bis(N-carbazolyl)-1,1′-biphenyl (CBP), 1,3-bis(carbazol-9-yl)benzene (mCP),
2,8-bis(diphenylphosphoryl)dibenzo[b,d]furan (PPF),
4,4′,4″-tris(carbazol-9-yl)-triphenylamine (TCTA), or
1,3,5-tris(1-phenyl-1H-benzo[d]imidazole-2-yl)benzene (TPBi). However, the embodiment of the present disclosure is not limited thereto, for example,
tris(8-hydroxyquinolinato)aluminum (Alq₃), 9,10-di(naphthalene-2-yl)anthracene (ADN),
2-tert-butyl-9,10-di(naphth-2-yl)anthracene (TBADN), distyrylarylene (DSA),
4,4′-bis(9-carbazolyl)-2,2′-dimethyl-biphenyl (CDBP),
2-methyl-9,10-bis(naphthalen-2-yl)anthracene (MADN), hexaphenyl cyclotriphosphazene (CP1), 1,4-bis(triphenylsilyl)benzene (UGH2),
hexaphenylcyclotrisiloxane (DPSiO₃), octaphenylcyclotetra siloxane (DPSiO₄), etc. may be utilized as a host material.

The emission layer EML may include the compound represented by Formula M-a. The compound represented by Formula M-a may be utilized as a phosphorescent dopant material.

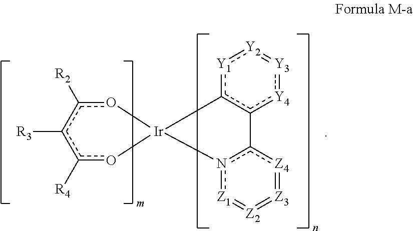

In Formula M-a, Y₁ to Y₄ and Z₁ to Z₄ may each independently be CR₁ or N, R₁ to R₄ may each independently be a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, and/or bonded to an adjacent group to form a ring. In Formula M-a, m is 0 or 1, and n is 2 or 3. In Formula M-a, when m is 0, n is 3, and when m is 1, n is 2.

The compound represented by Formula M-a may be utilized as a phosphorescent dopant.

The compound represented by Formula M-a may be represented by any one from among Compound M-a1 to Compound M-a25. However, Compounds M-a1 to M-a25 are examples, and the compound represented by Formula M-a is not limited to those represented by Compounds M-a1 to M-a25.

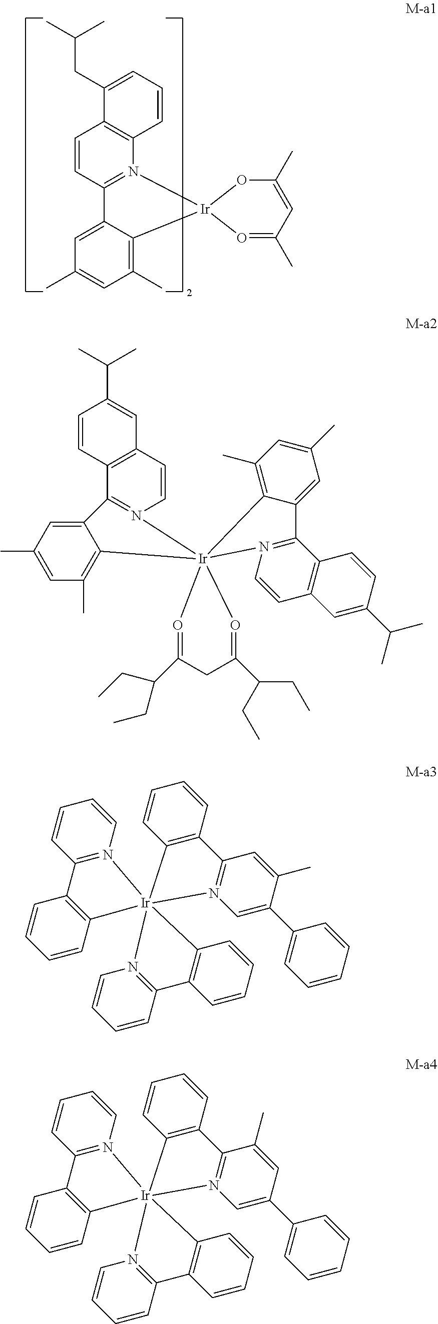

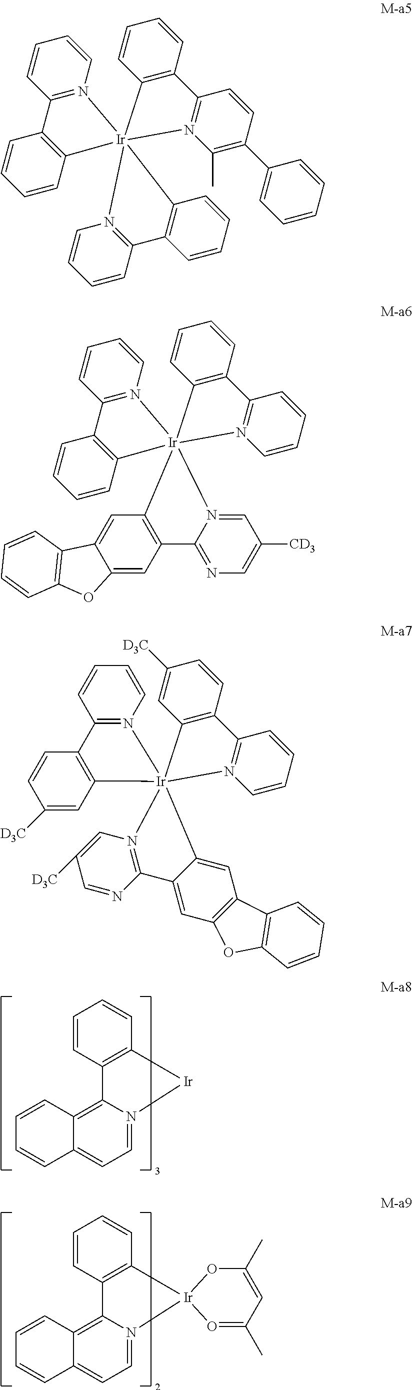

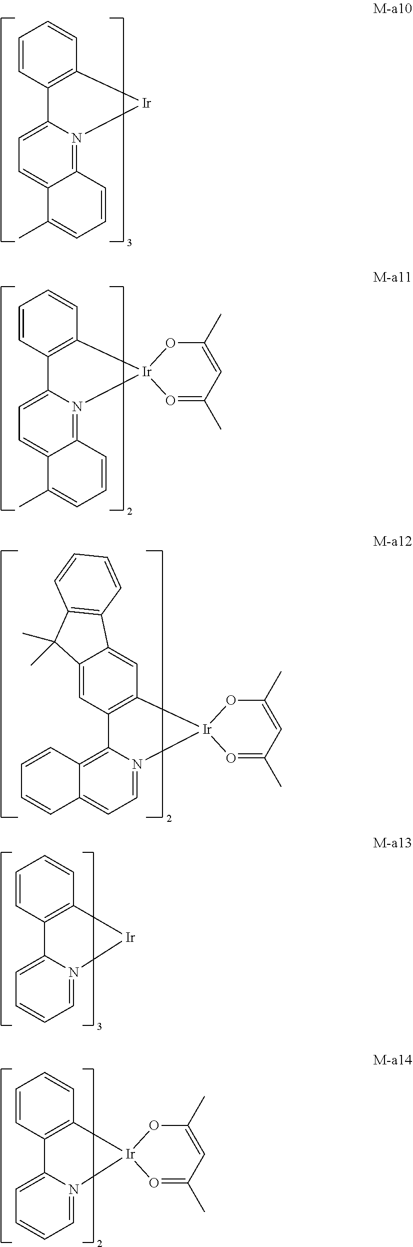

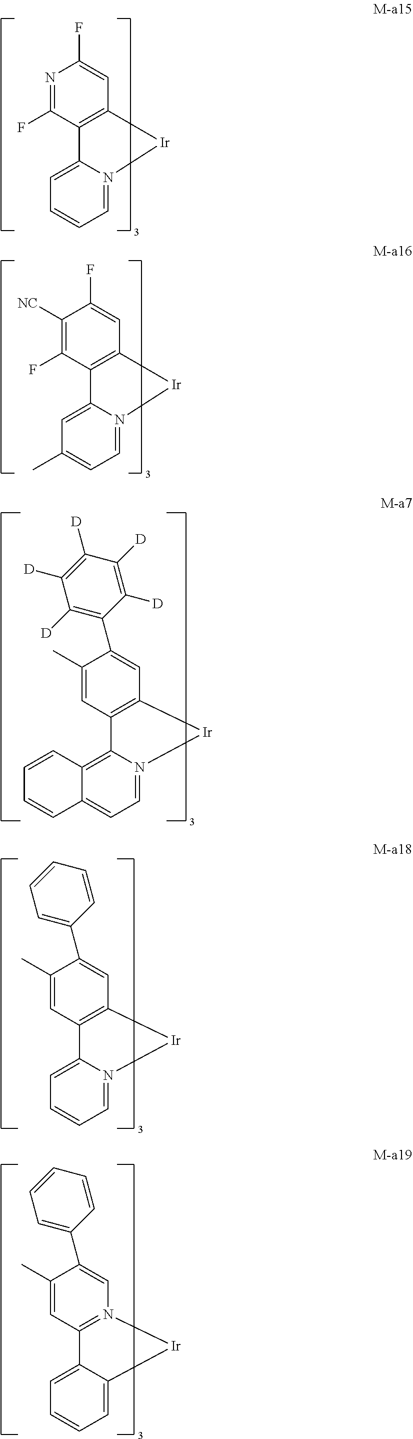

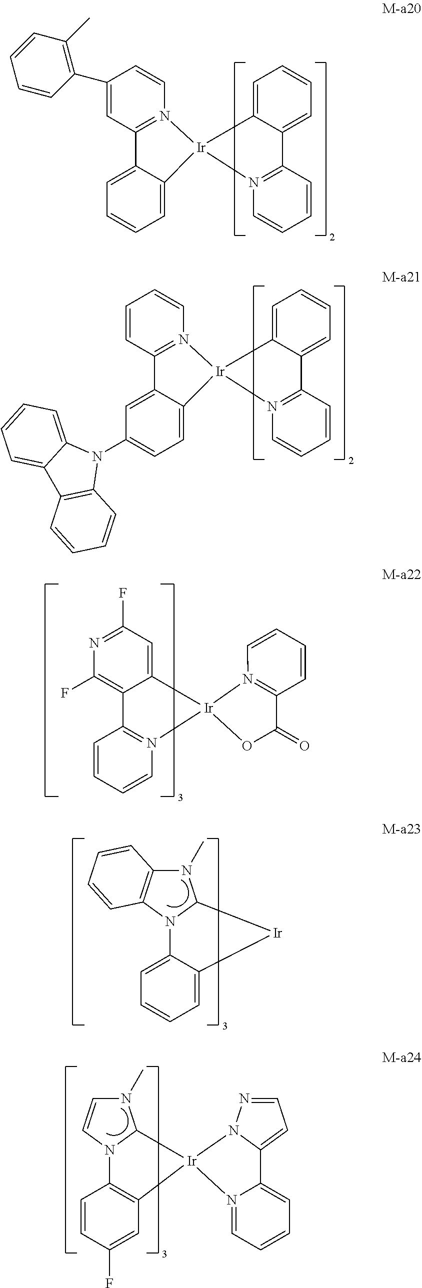

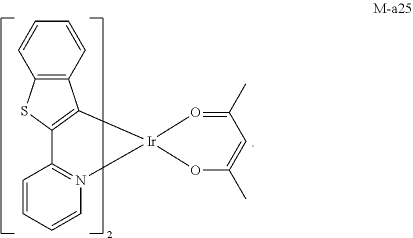

The emission layer EML may include a compound represented by any one from among Formula F-a to Formula F-c. The compound represented by Formula F-a or Formula F-c may be utilized as a fluorescence dopant material.

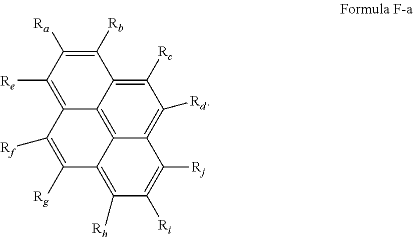

In Formula F-a, two selected from among Rₐ to Rⱼ may each independently be substituted with *—NAr₁Ar₂. Any remainder not substituted with *—NAr₁Ar₂ from among Rₐ to Rⱼ may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. In *—NAr₁Ar₂, Ar₁ and Ar₂ may each independently be a substituted or unsubstituted aryl group having 6 to 30 ring-forming carbon atoms, or a substituted or unsubstituted heteroaryl group having 2 to 30 ring-forming carbon atoms. For example, at least one of Ar₁ or Ar₂ may be a heteroaryl group containing O or S as a ring-forming atom.

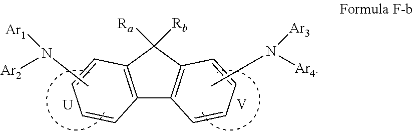

In Formula F-b, Rₐ and R_{b} may each independently be a hydrogen atom, a deuterium atom, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, and/or bonded to an adjacent group to form a ring. An to Ar₄ may each independently be a substituted or unsubstituted aryl group having 6 to 30 ring-forming carbon atoms, or a substituted or unsubstituted heteroaryl group having 2 to 30 ring-forming carbon atoms.

In Formula F-b, U and V may each independently be a substituted or unsubstituted hydrocarbon ring having 5 to 30 ring-forming carbon atoms, or a substituted or unsubstituted heterocycle having 2 to 30 ring-forming carbon atoms. At least one from among An to Ar₄ may be a heteroaryl group containing O or S as a ring-forming atom.

In Formula F-b, the number of rings represented by U and V may each independently be 0 or 1. For example, in Formula F-b, it refers to that when the number of U or V is 1, one ring indicated by U or V forms a fused ring at the designated part (e.g., a portion indicated by U or V), and when the number of U or V is 0, a ring indicated by U or V does not exist. For example, when the number of U is 0 and the number of V is 1, or when the number of U is 1 and the number of V is 0, the fused ring having a fluorene core in Formula F-b may be a cyclic compound having four rings. In some embodiments, when each number of U and V is 0, the fused ring in Formula F-b may be a cyclic compound having three rings. In some embodiments, when each number of U and V is 1, the fused ring having a fluorene core in Formula F-b may be a cyclic compound having five rings.

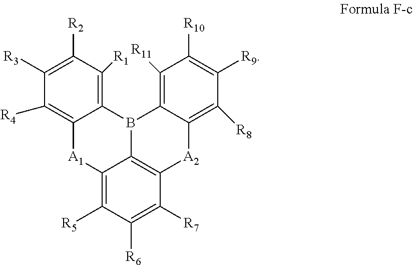

In Formula F-c, A₁ and A₂ may each independently be O, S, Se, or NRₘ, and Rₘ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. R₁ to R₁₁ may each independently be a hydrogen atom, a deuterium atom, a halogen atom, a cyano group, a substituted or unsubstituted amine group, a substituted or unsubstituted boryl group, a substituted or unsubstituted oxy group, a substituted or unsubstituted thio group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and/or bonded to an adjacent group to form a ring.

In Formula F-c, A₁ and A₂ may each independently be bonded to substituents of an adjacent ring to form a condensed ring. For example, when A₁ and A₂ may each independently be NRₘ, A₁ may be bonded to R₄ or R₅ to form a ring. In some embodiments, A₂ may be bonded to R₇ or R₈ to form a ring.

In an embodiment, the emission layer EML may further include, as a suitable dopant material, one or more styryl derivatives (e.g.,
1,4-bis[2-(3-N-ethylcarbazoryl)vinyl]benzene (BCzVB),
4-(di-p-tolylamino)-4′-[(di-p-tolylamino)styryl]stilbene (DPAVB), and
N-(4-((E)-2-(6-((E)-4-(diphenylamino)styryl)naphthalen-2-yl)vinyl)phenyl)-N-phenylbenz enamine (N-BDAVBi), 4,4′-bis[2-(4-(N,N-diphenylamino)phenyl)vinyl]biphenyl (DPAVBi), perylene and the derivatives thereof (e.g., 2,5,8,11-tetra-t-butylperylene (TBP)), pyrene and the derivatives thereof (e.g., 1,1-dipyrene, 1,4-dipyrenylbenzene, 1,4-bis(N,N-diphenylamino)pyrene), etc.

The emission layer EML may further include a suitable phosphorescence dopant material. For example, a metal complex containing iridium (Ir), platinum (Pt), osmium (Os), aurum (Au), titanium (Ti), zirconium (Zr), hafnium (Hf), europium (Eu), terbium (Tb), or thulium (Tm) may be utilized as a phosphorescent dopant. For example, iridium(III) bis(4,6-difluorophenylpyridinato-N,C2′)picolinate (Firpic), bis(2,4-difluorophenylpyridinato)-tetrakis(1-pyrazolyl)borate iridium(III) (Fir6), or platinum octaethyl porphyrin (PtOEP) may be utilized as a phosphorescent dopant. However, the embodiment of the present disclosure is not limited thereto.

The emission layer EML may include a quantum dot material. The core of the quantum dot may be selected from among a Group II-VI compound, a Group III-VI compound, a Group I-III-IV compound, a Group III-V compound, a Group III-II-V compound, a Group IV-VI compound, a Group IV element, a Group IV compound, and a combination thereof.

The Group II-VI compound may be selected from the group consisting of a binary compound selected from the group consisting of CdSe, CdTe, CdS, ZnS, ZnSe, ZnTe, ZnO, HgS, HgSe, HgTe, MgSe, MgS, and a mixture thereof, a ternary compound selected from the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and a mixture thereof, and a quaternary compound selected from the group consisting of HgZnTeS, CdZnSeS, CdZnSeTe, CdZnSTe, CdHgSeS, CdHgSeTe, CdHgSTe, HgZnSeS, HgZnSeTe, HgZnSTe, and a mixture thereof.

The Group III-VI compound may include a binary compound such as In₂S₃ and/or In₂Se₃, a ternary compound such as InGaS₃ and/or InGaSe₃, or any combination thereof.

The Group I-III-VI compound may be selected from a ternary compound selected from the group consisting of AgInS, AgInS₂, CulnS, CuInS₂, AgGaS₂, CuGaS₂ CuGaO₂, AgGaO₂, AgAIO₂, and a mixture thereof, and a quaternary compound such as AgInGaS₂ and/or CuInGaS₂.

The Group III-V compound may be selected from the group consisting of a binary compound selected from the group consisting of GaN, GaP, GaAs, GaSb, AlN, AIP, AlAs, AlSb, InN, InP, InAs, InSb, and a mixture thereof, a ternary compound selected from the group consisting of GaNP, GaNAs, GaNSb, GaPAs, GaPSb, AlNP, AlNAs, AlNSb, AlPAs, AlPSb, InGaP, InAlP, InNP, InNAs, InNSb, InPAs, InPSb, and a mixture thereof, and a quaternary compound selected from the group consisting of GaAlNP, GaAlNAs, GaAlNSb, GaAIPAs, GaAIPSb, GaInNP, GaInNAs, GaInNSb, GaInPAs, GaInPSb, InAlNP, InAlNAs, InAlNSb, InAlPAs, InAlPSb, and a mixture thereof. In some embodiments, the Group III-V compound may further include a Group II metal. For example, InZnP, etc. may be selected as a Group III-II-V compound.

The Group IV-VI compound may be selected from the group consisting of a binary compound selected from the group consisting of SnS, SnSe, SnTe, PbS, PbSe, PbTe, and a mixture thereof, a ternary compound selected from the group consisting of SnSeS, SnSeTe, SnSTe, PbSeS, PbSeTe, PbSTe, SnPbS, SnPbSe, SnPbTe, and a mixture thereof, and a quaternary compound selected from the group consisting of SnPbSSe, SnPbSeTe, SnPbSTe, and a mixture thereof. The Group IV element may be selected from the group consisting of Si, Ge, and a mixture thereof. The Group IV compound may be a binary compound selected from the group consisting of SiC, SiGe, and a mixture thereof.

In this case, the binary compound, the ternary compound, and/or the quaternary compound may be present in a particle with a substantially uniform concentration distribution, or may be present in substantially the same particle with a partially different concentration distribution. In some embodiments, the quantum dot may have a core/shell structure in which one quantum dot is around (e.g., surrounds) the other quantum dot. The core/shell structure may have a concentration gradient in which the concentration of elements present in the shell decreases toward the core.

In some embodiments, the quantum dot may have the above-described core/shell structure including a core containing nanocrystals and a shell around (e.g., surrounding) the core. The shell of the quantum dot may serve as a protection layer to prevent or reduce the chemical deformation of the core to maintain semiconductor properties, and/or a charging layer to impart electrophoresis properties to the quantum dot. The shell may be a single layer or a multilayer. An example of the shell of the quantum dot may include a metal or non-metal oxide, a semiconductor compound, or a combination thereof.

For example, the metal or non-metal oxide may be a binary compound such as SiO₂, Al₂O₃, TiO₂, ZnO, MnO, Mn₂O₃, Mn₃O₄, CuO, FeO, Fe₂O₃, Fe₃O₄, CoO, CO₃O₄, and/or NiO, or a ternary compound such as MgAl₂O₄, CoFe₂O₄, NiFe₂O₄, and/or CoMn₂O₄, but the embodiment of the present disclosure is not limited thereto.

Also, the semiconductor compound may be, for example, CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, etc., but the embodiment of the present disclosure is not limited thereto.

The quantum dot may have a full width of half maximum (FWHM) of a light emitting wavelength spectrum of about 45 nm or less, about 40 nm or less, or about 30 nm or less, and color purity or color reproducibility may be improved in these range. In some embodiments, light emitted through such a quantum dot is emitted in all directions, and thus a wide viewing angle may be improved.

In some embodiments, although the form of the quantum dot is not particularly limited as long as it is a form commonly utilized in the art, more specifically, the quantum dot in the form of one or more spherical, pyramidal, multi-arm, and/or cubic nanoparticles, nanotubes, nanowires, nanofibers, nanoplate particles, etc. may be utilized.

A quantum dot may control the color of emitted light according to the particle size thereof and thus the quantum dot may have one or more suitable light emission colors such as blue, red, and/or green.

In each light emitting device ED of embodiments illustrated in FIGS. 3 to 6, the electron transport region ETR is provided on the emission layer EML. The electron transport region ETR may include at least one of the hole blocking layer HBL, the electron transport layer ETL, or the electron injection layer EIL, but the embodiment of the present disclosure is not limited thereto.

The electron transport region ETR may have a single layer formed of a single material, a single layer formed of a plurality of different materials, or a multilayer structure including a plurality of layers formed of a plurality of different materials.

For example, the electron transport region ETR may have a single layer structure of the electron injection layer EIL or the electron transport layer ETL, and may have a single layer structure formed of an electron injection material and an electron transport material. In some embodiments, the electron transport region ETR may have a single layer structure formed of a plurality of different materials, or may have a structure in which an electron transport layer ETL/an electron injection layer EIL, or a hole blocking layer HBL/an electron transport layer ETL/an electron injection layer EIL, that are stacked in the respective stated order from the emission layer EML, but the embodiment of the present disclosure is not limited thereto. The electron transport region ETR may have a thickness, for example, from about 1,000 Å to about 1,500 Å.

The electron transport region ETR may be formed by utilizing one or more suitable methods such as a vacuum deposition method, a spin coating method, a cast method, a Langmuir-Blodgett (LB) method, an inkjet printing method, a laser printing method, and/or a laser induced thermal imaging (LITI) method.

The electron transport region ETR may include a compound represented by Formula ET-1:

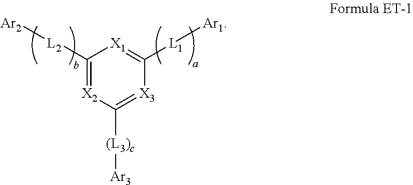

In Formula ET-1, at least one from among X₁ to X₃ may be N, and any remainder thereof may be CRₐ. Rₐ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. An to Ar₃ may each independently be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

In Formula ET-1, a to c may each independently be an integer of 0 to 10. In Formula ET-1, L₁ to L₃ may each independently be a direct linkage, a substituted or unsubstituted arylene group having 6 to 30 ring-forming carbon atoms, or a substituted or unsubstituted heteroarylene group having 2 to 30 ring-forming carbon atoms. In some embodiments, when a to c are an integer of 2 or more, L₁ to L₃ may each independently be a substituted or unsubstituted arylene group having 6 to 30 ring-forming carbon atoms, or a substituted or unsubstituted heteroarylene group having 2 to 30 ring-forming carbon atoms.

The electron transport region ETR may include an anthracene-based compound. However, the embodiment of the present disclosure is not limited thereto, and the electron transport region ETR may include, for example,
tris(8-hydroxyquinolinato)aluminum (Alq₃), 1,3,5-tri[(3-pyridyl)-phen-3-yl]benzene,
2,4,6-tris(3′-(pyridin-3-yl)biphenyl-3-yl)-1,3,5-triazine,
2-(4-(N-phenylbenzoimidazol-1-yl)phenyl)-9,10-dinaphthylanthracene,
1,3,5-tri(1-phenyl-1H-benzo[d]imidazol-2-yl)benzene (TPBi),
2,9-dimethyl-4,7-diphenyl-1,10-phenanthroline (BCP),
4,7-diphenyl-1,10-phenanthroline (Bphen),
3-(4-biphenylyl)-4-phenyl-5-tert-butylphenyl-1,2,4-triazole (TAZ),
4-(naphthalen-1-yl)-3,5-diphenyl-4H-1,2,4-triazole (NTAZ),
2-(4-biphenylyl)-5-(4-tert-butylphenyl)-1,3,4-oxadiazole (tBu-PBD),
bis(2-methyl-8-quinolinolato-N1,O8)-(1,1′-biphenyl-4-olato)aluminum (BAlq),
berylliumbis(benzoquinolin-10-olate (Bebq₂), 9,10-di(naphthalene-2-yl)anthracene
(ADN), 1,3-Bis[3,5-di(pyridin-3-yl)phenyl]benzene (BmPyPhB), or a mixture thereof.

The electron transport region ETR may include at least one from among Compound ET1 to Compound ET36:

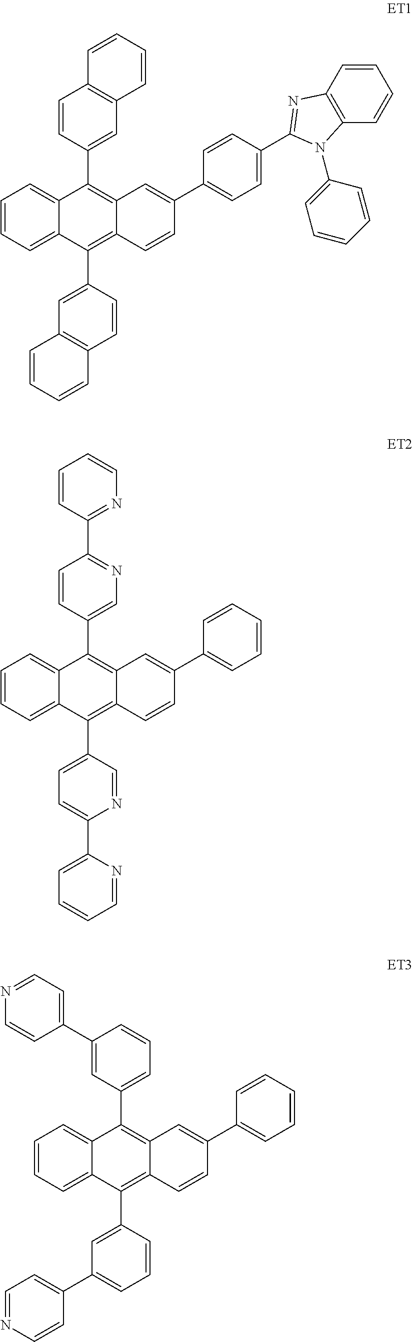 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

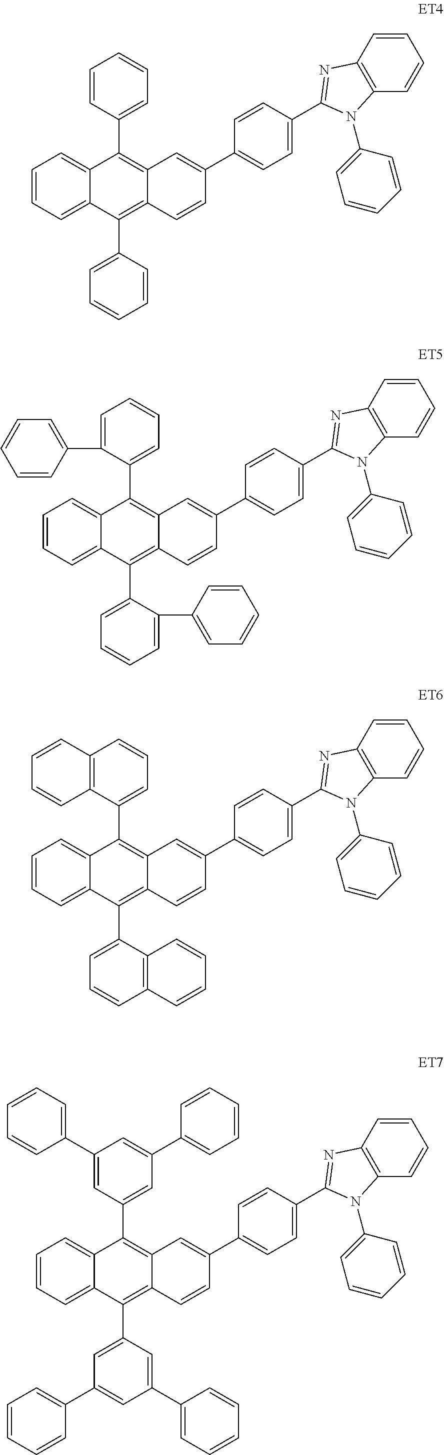 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1 ; c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1]

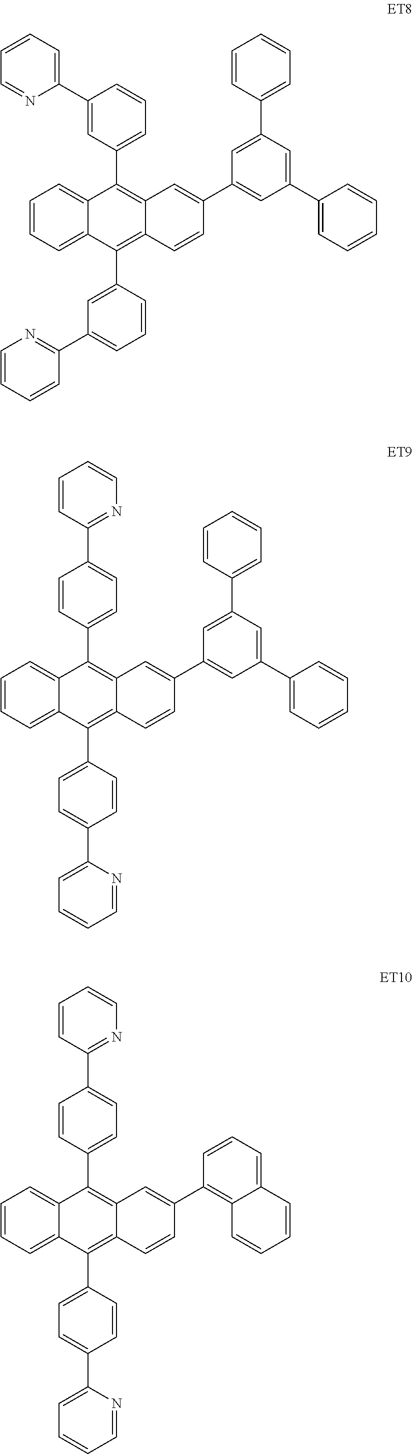 [SMILES: c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1 ; c1ccc(-c2ccc(-c3c4ccccc4c(-c4ccc(-c5ccccn5)cc4)c4cc(-c5cccc6ccccc56)ccc34)cc2)nc1]

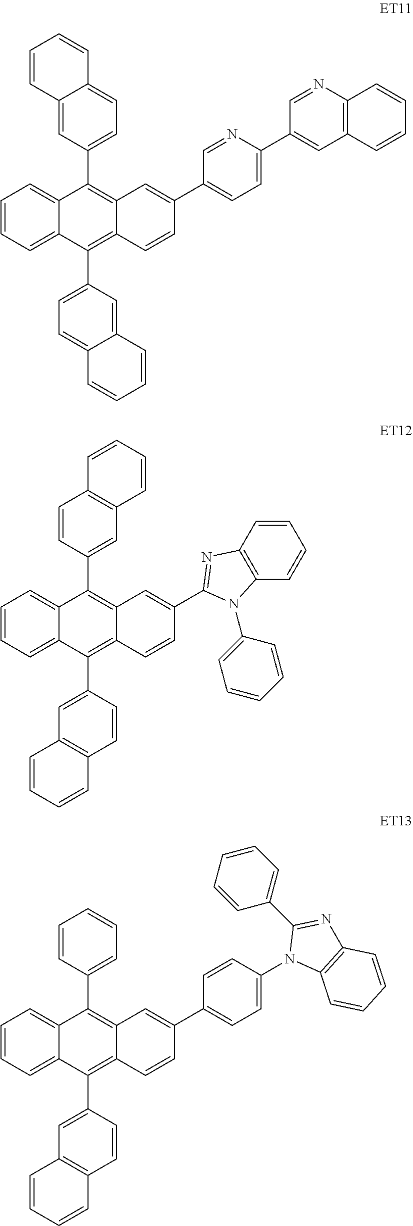

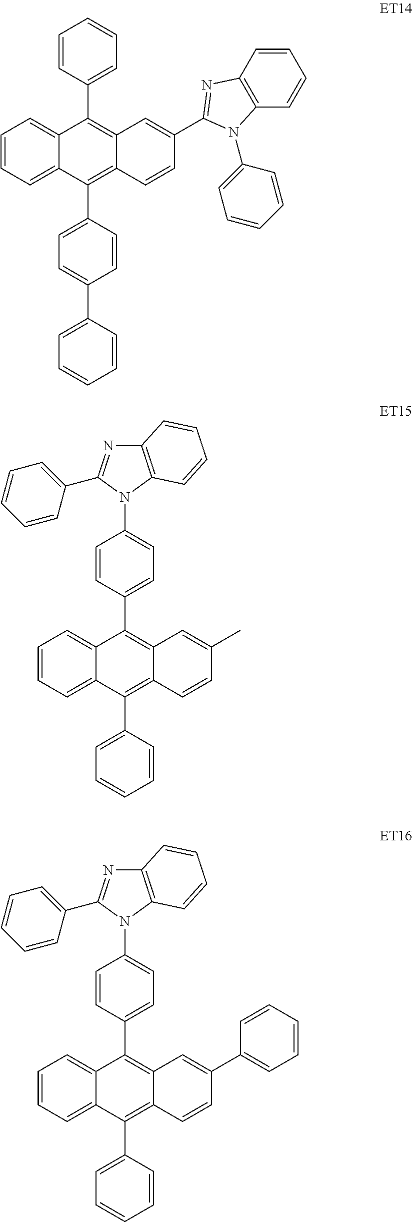 [SMILES: c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1 ; c1ccc(-c2ccc3c(-c4ccccc4)c4ccccc4c(-c4ccc(-n5c(-c6ccccc6)nc6ccccc65)cc4)c3c2)cc1]

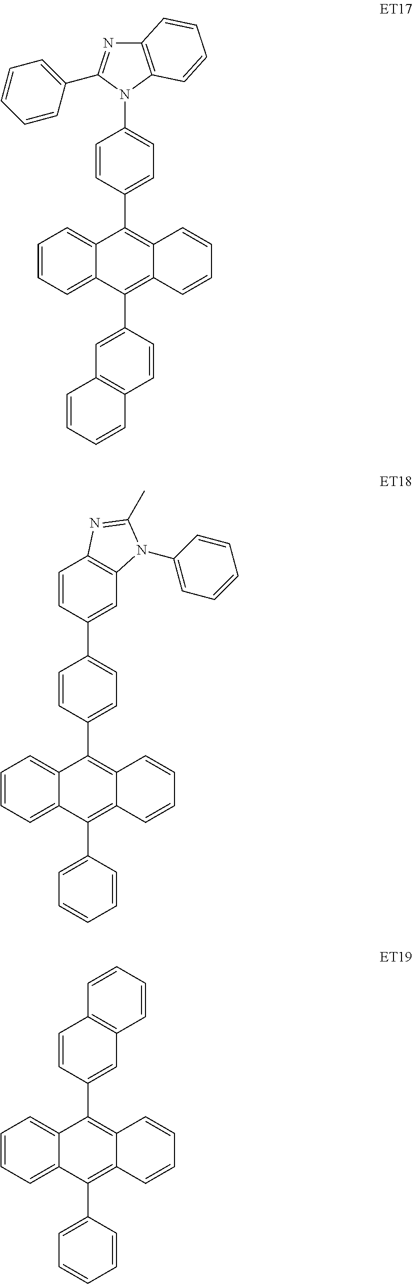

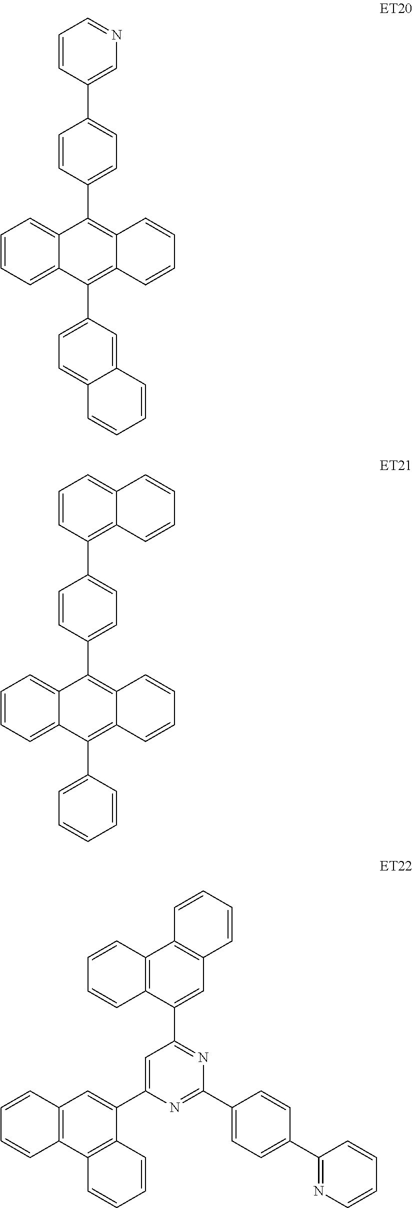

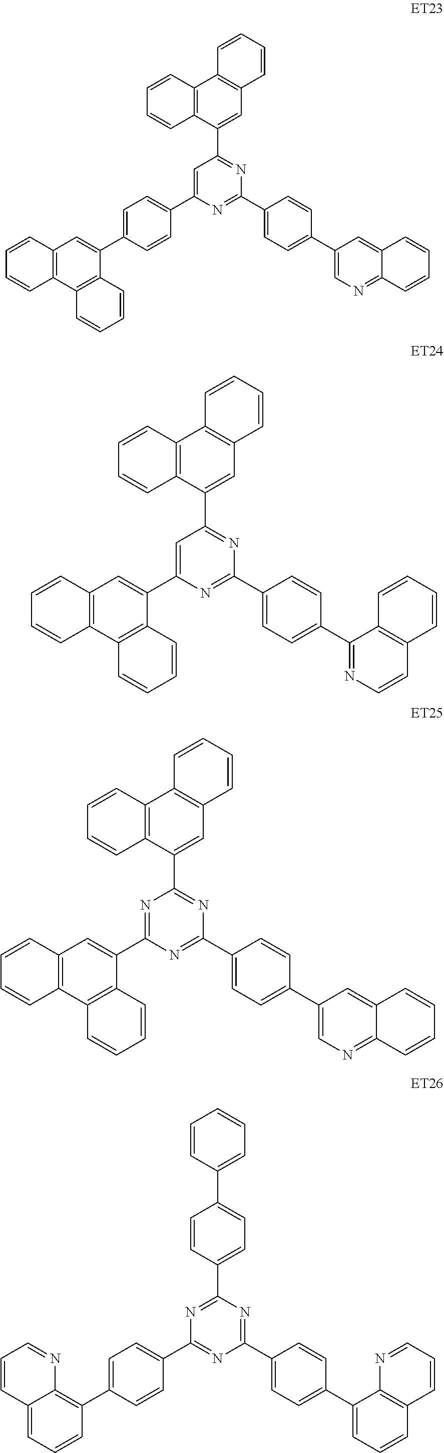 [SMILES: c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1 ; c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1]

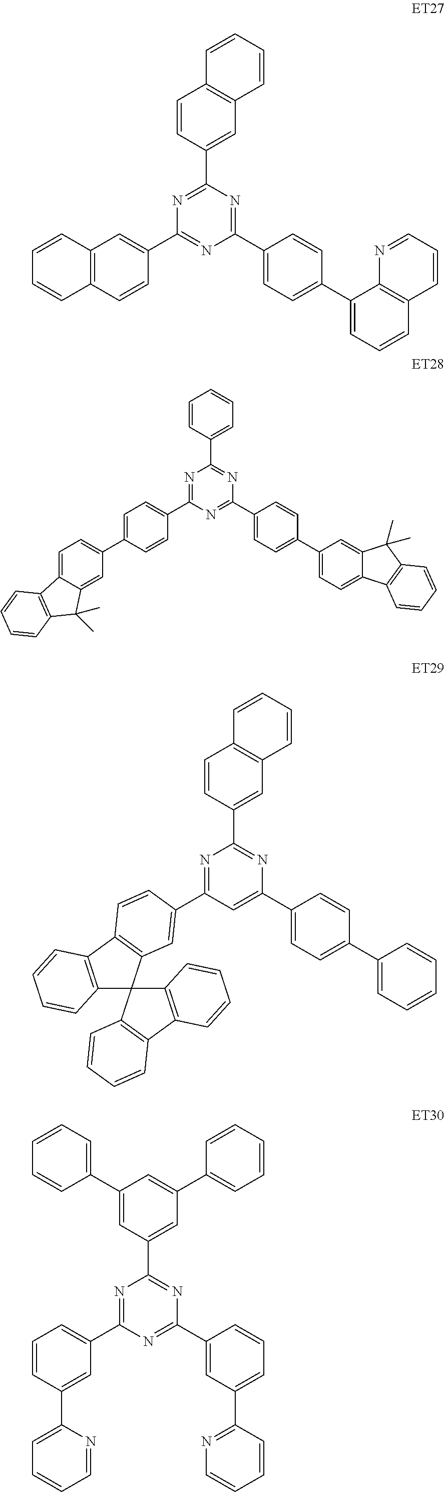 [SMILES: c1ccc2cc(-c3nc(-c4ccc(-c5cccc6cccnc56)cc4)nc(-c4ccc5ccccc5c4)n3)ccc2c1 ; CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1]

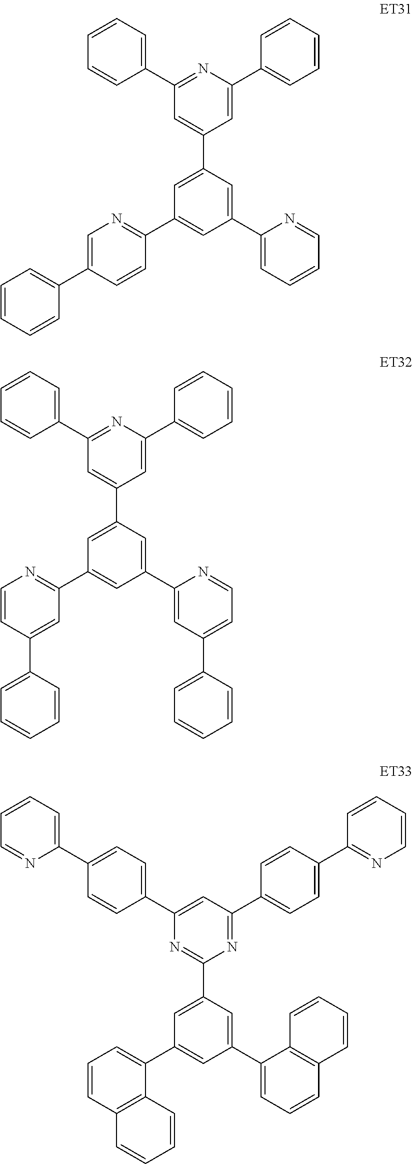

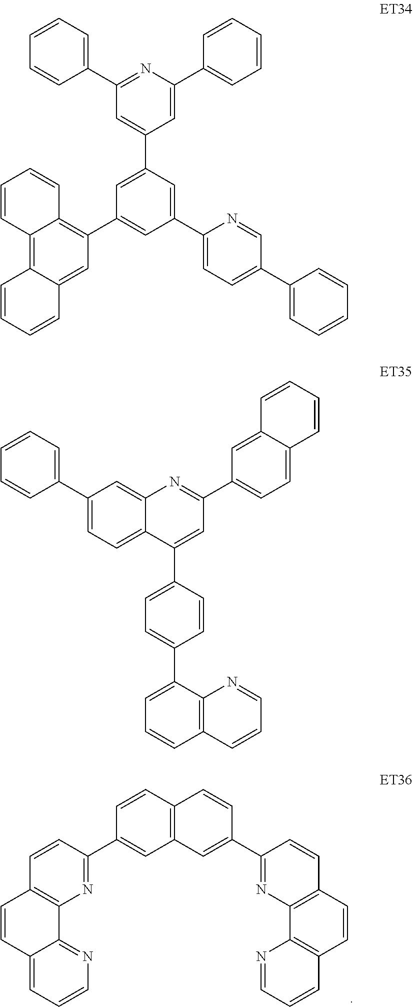 [SMILES: c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

In some embodiments, the electron transport regions ETR may include a metal halide such as LiF, NaCl, CsF, RbCl, RbI, CuI, and/or KI, a lanthanide metal such as Yb, and a co-deposited material of the metal halide and the lanthanide metal. For example, the electron transport region ETR may include KI:Yb, RbI:Yb, LiF:Yb, etc. as a co-deposited material. In some embodiments, the electron transport region ETR may be formed utilizing a metal oxide such as Li₂O and/or BaO, and/or 8-hydroxyl-lithium quinolate (Liq), etc., but the embodiment of the present disclosure is not limited thereto. The electron transport region ETR may also be formed of a mixture material of an electron transport material and an insulating organometallic salt. The organometallic salt may be a material having an energy band gap of about 4 eV or more. For example, the organometallic salt may include, a metal acetate, a metal benzoate, a metal acetoacetate, a metal acetylacetonate, and/or a metal stearate.

The electron transport region ETR may further include at least one of
2,9-dimethyl-4,7-diphenyl-1,10-phenanthroline (BCP),
diphenyl(4-(triphenylsilyl)phenyl)phosphine oxide (TSPO1), or
4,7-diphenyl-1,10-phenanthroline (Bphen) in addition to the above-described materials, but the embodiment of the present disclosure is not limited thereto.

The electron transport region ETR may include the above-described compounds of the electron transport region ETR in at least one of the electron injection layer EIL, the electron transport layer ETL, or the hole blocking layer HBL.

When the electron transport region ETR includes the electron transport layer ETL, the electron transport layer ETL may have a thickness of about 100 Ato about 1,000 Å, for example, about 150 Å to about 500 Å. When the thickness of the electron transport layer ETL satisfies the aforementioned range, satisfactory electron transport characteristics may be obtained without a substantial increase in driving voltage. When the electron transport region ETR includes the electron injection layer EIL, the electron injection layer EIL may have a thickness of about 1 Å to about 100 Å, for example, about 3 Å to about 90 Å. When the thickness of the electron injection layer EIL satisfies the above-described range, satisfactory electron injection characteristics may be obtained without a substantial increase in driving voltage.

The second electrode EL2 is provided on the electron transport region ETR. The second electrode EL2 may be a common electrode. The second electrode EL2 may be a cathode or an anode, but the embodiment of the present disclosure is not limited thereto. For example, when the first electrode EL1 is an anode, the second electrode EL2 may be a cathode, and when the first electrode EL1 is a cathode, the second electrode EL2 may be an anode.

The second electrode EL2 may be a transmissive electrode, a transflective electrode, or a reflective electrode. When the second electrode EL2 is the transmissive electrode, the second electrode EL2 may be formed of a transparent metal oxide, for example, indium tin oxide (ITO), indium zinc oxide (IZO), zinc oxide (ZnO), indium tin zinc oxide (ITZO), etc.

When the second electrode EL2 is the transflective electrode or the reflective electrode, the second electrode EL2 may include Ag, Mg, Cu, Al, Pt, Pd, Au, Ni, Nd, Ir, Cr, Li, Ca, LiF/Ca, LiF/Al, Mo, Ti, Yb, W, or a compound or mixture thereof (e.g., AgMg, AgYb, or MgAg). In some embodiments, the second electrode EL2 may have a multilayer structure including a reflective film or a transflective film formed of the above-described materials, and a transparent conductive film formed of ITO, IZO, ZnO, ITZO, etc. For example, the second electrode EL2 may include the above-described metal materials, combinations of at least two metal materials of the above-described metal materials, oxides of the above-described metal materials, and/or the like.

the second electrode EL2 may be connected with an auxiliary electrode. When the second electrode EL2 is connected with the auxiliary electrode, the resistance of the second electrode EL2 may decrease.

In some embodiments, a capping layer CPL may further be disposed on the second electrode EL2 of the light emitting device ED of an embodiment. The capping layer CPL may include a multilayer or a single layer.

In an embodiment, the capping layer CPL may be an organic layer or an inorganic layer. For example, when the capping layer CPL contains an inorganic material, the inorganic material may include an alkaline metal compound (for example, LiF), an alkaline earth metal compound (for example, MgF₂), SiON, SiNₓ, SiOy, etc.

For example, when the capping layer CPL contains an organic material, the organic material may include
2,2′-dimethyl-N,N′-di-[(1-naphthyl)-N,N′-diphenyl]-1,1′-biphenyl-4,4′-diamine(α-NPD), NPB, TPD, m-MTDATA, Alq₃, CuPc,
N4,N4,N4′,N4′-tetra(biphenyl-4-yl)biphenyl-4,4′-diamine (TPD15),
4,4′,4″-tris(carbazol-9-yl)triphenylamine (TCTA), etc., and/or may include an epoxy resin, and/or an acrylate such as a methacrylate. However, the embodiment of the present disclosure is not limited thereto, and the capping layer CPL may include at least one from among Compounds P1 to P5:

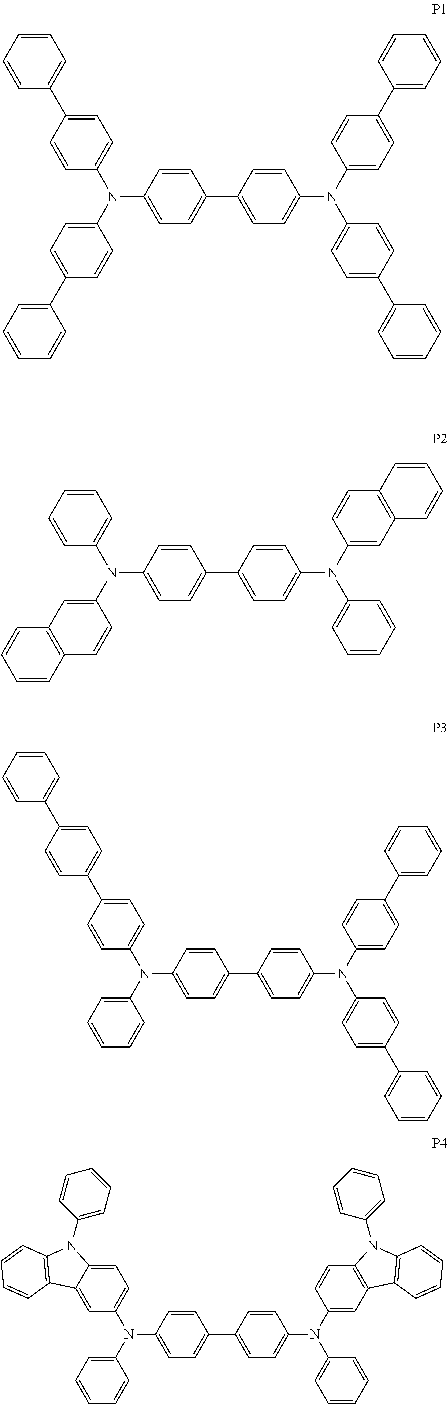 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1]

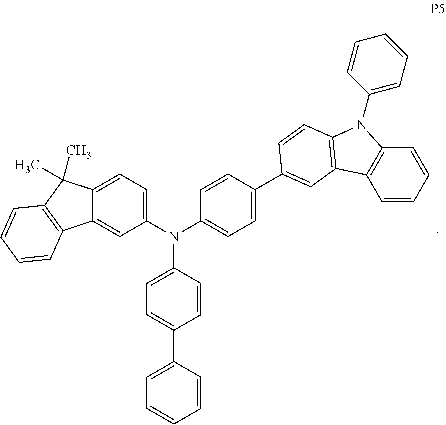 [SMILES: CC1(C)c2ccccc2-c2cc(N(c3ccc(-c4ccccc4)cc3)c3ccc(-c4ccc5c(c4)c4ccccc4n5-c4ccccc4)cc3)ccc21]

In some embodiments, the refractive index of the capping layer CPL may be about 1.6 or more. For example, the refractive index of the capping layer CPL may be 1.6 or more with respect to light in a wavelength range of about 550 nm to about 660 nm.

Each of FIGS. 7 and 8 is a cross-sectional view of a display apparatus according to an embodiment of the present disclosure. Hereinafter, in describing the display apparatuses of embodiments with reference to FIGS. 7 and 8, the duplicated features which have been described in FIGS. 1 to 6 are not described again, but their differences will be mainly described.

Referring to FIG. 7, the display apparatus DD according to an embodiment may include a display panel DP including a display apparatus layer DP-ED, a light control layer CCL disposed on the display panel DP, and a color filter layer CFL.

In an embodiment illustrated in FIG. 7, the display panel DP may include a base layer BS, a circuit layer DP-CL provided on the base layer BS, and the display device layer DP-ED, and the display device layer DP-ED may include a light emitting device ED.

The light emitting device ED may include a first electrode EL1, a hole transport region HTR disposed on the first electrode EL1, an emission layer EML disposed on the hole transport region HTR, an electron transport region ETR disposed on the emission layer EML, and a second electrode EL2 disposed on the electron transport region ETR. In some embodiments, the structures of the light emitting devices of FIGS. 3 to 6 as described may be equally applied to the structure of the light emitting device ED illustrated in FIG. 7.

The emission layer EML of the light emitting device ED included in the display apparatus DD-a according to an embodiment may include the above-described fused polycyclic compound of an embodiment.

Referring to FIG. 7, the emission layer EML may be disposed in an opening OH defined in a pixel defining film PDL. For example, the emission layer EML which is divided by the pixel defining film PDL and provided corresponding to each light emitting regions PXA-R, PXA-G, and PXA-B may be to emit light in substantially the same wavelength range. In the display apparatus DD of an embodiment, the emission layer EML may be to emit blue light. In some embodiments, unlike the configuration illustrated, in an embodiment, the emission layer EML may be provided as a common layer in the entire light emitting regions PXA-R, PXA-G, and PXA-B.

The light control layer CCL may be disposed on the display panel DP. The light control layer CCL may include a light conversion body. The light conversion body may be a quantum dot, a phosphor, and/or the like. The light conversion body may transform the wavelength of light provided and then emit that light. For example, the light control layer CCL may a layer containing the quantum dot or a layer containing the phosphor.

The light control layer CCL may include a plurality of light control parts CCP1, CCP2 and CCP3. The light control parts CCP1, CCP2, and CCP3 may be spaced apart from each other.

Referring to FIG. 7, divided patterns BMP may be disposed between the light control parts CCP1, CCP2 and CCP3 which are spaced apart from each other, but the embodiment of the present disclosure is not limited thereto. FIG. 7 illustrates that the divided patterns BMP do not overlap the light control parts CCP1, CCP2 and CCP3, but in an embodiment, at least a portion of the edges of the light control parts CCP1, CCP2 and CCP3 may overlap the divided patterns BMP.

The light control layer CCL may include a first light control part CCP1 containing a first quantum dot QD1 which converts a first color light provided from the light emitting device ED into a second color light, a second light control part CCP2 containing a second quantum dot QD2 which converts the first color light into a third color light, and a third light control part CCP3 which transmits the first color light.

In an embodiment, the first light control part CCP1 may provide red light as the second color light, and the second light control part CCP2 may provide green light as the third color light. The third light control part CCP3 may provide blue light by transmitting the blue light as the first color light provided from the light emitting device ED. For example, the first quantum dot QD1 may be a red quantum dot, and the second quantum dot QD2 may be a green quantum dot. The same as described may be applied with respect to the quantum dots QD1 and QD2.

In some embodiments, the light control layer CCL may further include a scatterer SP. The first light control part CCP1 may include the first quantum dot QD1 and the scatterer SP, the second light control part CCP2 may include the second quantum dot QD2 and the scatterer SP, and the third light control part CCP3 may not include (e.g., may exclude) any quantum dot but include the scatterer SP.

The scatterer SP may be inorganic particles. For example, the scatterer SP may include at least one of TiO₂, ZnO, Al₂O₃, SiO₂, or hollow sphere silica. The scatterer SP may include any one from among TiO₂, ZnO, Al₂O₃, SiO₂, and hollow sphere silica, or may be a mixture of at least two materials selected from among TiO₂, ZnO, Al₂O₃, SiO₂, and hollow sphere silica.

The first light control part CCP1, the second light control part CCP2, and the third light control part CCP3 each may include a corresponding one of the base resins BR1, BR2, and BR3 in which the quantum dots QD1 and QD2 and the scatterer SP are dispersed. In an embodiment, the first light control part CCP1 may include the first quantum dot QD1 and the scatterer SP dispersed in a first base resin BR1, the second light control part CCP2 may include the second quantum dot QD2 and the scatterer SP dispersed in a second base resin BR2, and the third light control part CCP3 may include the scatterer SP dispersed in a third base resin BR3. The base resins BR1, BR2, and BR3 are media in which the quantum dots QD1 and QD2 and the scatterer SP are dispersed, and may be formed of one or more suitable resin compositions, which may be generally referred to as a binder. For example, the base resins BR1, BR2, and BR3 may each be one or more acrylic-based resins, urethane-based resins, silicone-based resins, epoxy-based resins, etc. The base resins BR1, BR2, and BR3 may each be transparent resins. In an embodiment, the first base resin BR1, the second base resin BR2, and the third base resin BR3 may be the same as or different from each other.

The light control layer CCL may include a barrier layer BFL1. The barrier layer BFL1 may serve to prevent or reduce the penetration of moisture and/or oxygen (hereinafter, referred to as ‘moisture/oxygen’). The barrier layer BFL1 may be disposed on the light control parts CCP1, CCP2, and CCP3 to block or reduce the exposure of the light control parts CCP1, CCP2 and CCP3 to moisture/oxygen. In some embodiments, the barrier layer BFL1 may cover the light control parts CCP1, CCP2, and CCP3. In some embodiments, the barrier layer BFL2 may be provided between the light control parts CCP1, CCP2, and CCP3 and the color filter layer CFL.

The barrier layers BFL1 and BFL2 may include at least one inorganic layer. For example, the barrier layers BFL1 and BFL2 may include an inorganic material. For example, the barrier layers BFL1 and BFL2 may include a silicon nitride, an aluminum nitride, a zirconium nitride, a titanium nitride, a hafnium nitride, a tantalum nitride, a silicon oxide, an aluminum oxide, a titanium oxide, a tin oxide, a cerium oxide, a silicon oxynitride, and/or a metal thin film which secures a transmittance, etc. In some embodiments, the barrier layers BFL1 and BFL2 may further include an organic film. The barrier layers BFL1 and BFL2 may be formed of a single layer or a plurality of layers.

In the display apparatus DD-a of an embodiment, the color filter layer CFL may be disposed on the light control layer CCL. For example, the color filter layer CFL may be directly disposed on the light control layer CCL. In this case, the barrier layer BFL2 may not be provided.

The color filter layer CFL may include a light shielding part BM and color filters CF1, CF2, and CF3. The color filter layer CFL may include a first filter CF1 configured to transmit the second color light, a second filter CF2 configured to transmit the third color light, and a third filter CF3 configured to transmit the first color light. For example, the first filter CF1 may be a red filter, the second filter CF2 may be a green filter, and the third filter CF3 may be a blue filter. The filters CF1, CF2, and CF3 each may include a polymeric photosensitive resin and a pigment and/or dye. The first filter CF1 may include a red pigment and/or dye, the second filter CF2 may include a green pigment and/or dye, and the third filter CF3 may include a blue pigment and/or dye. In some embodiments, the embodiment of the present disclosure is not limited thereto, and the third filter CF3 may not include (e.g., may exclude) any pigment or dye. The third filter CF3 may include a polymeric photosensitive resin and may not include (e.g., may exclude) any pigment or dye. The third filter CF3 may be transparent. The third filter CF3 may be formed of a transparent photosensitive resin.

Furthermore, in an embodiment, the first filter CF1 and the second filter CF2 may each be a yellow filter. In an embodiment, the first filter CF1 and the second filter CF2 may not be separated but may be provided as one filter.

The light shielding part BM may be a black matrix. The light shielding part BM may include an organic light shielding material or an inorganic light shielding material containing a black pigment or dye. The light shielding part BM may prevent or reduce light leakage, and may separate boundaries between the adjacent filters CF1, CF2, and CF3. In some embodiments, the light shielding part BM may be formed of a blue filter.

The first to third filters CF1, CF2, and CF3 may be disposed corresponding to the red light emitting region PXA-R, the green light emitting region PXA-G, and the blue light emitting region PXA-B, respectively.

A base substrate BL may be disposed on the color filter layer CFL. The base substrate BL may be a member which provides a base surface in which the color filter layer CFL, the light control layer CCL, and/or the like are disposed. The base substrate BL may be a glass substrate, a metal substrate, a plastic substrate, etc. However, the embodiment of the present disclosure is not limited thereto, and the base substrate BL may be an inorganic layer, an organic layer, or a composite material layer. In some embodiments, unlike the configuration illustrated, in an embodiment, the base substrate BL may not be provided.

FIG. 8 is a cross-sectional view illustrating a portion of a display apparatus according to an embodiment. In the display apparatus DD-TD of an embodiment, the light emitting device ED-BT may include a plurality of light emitting structures OL-B1, OL-B2, and OL-B3. The light emitting device ED-BT may include a first electrode EL1 and a second electrode EL2 which face each other, and the plurality of light emitting structures OL-B1, OL-B2, and OL-B3 sequentially stacked in the thickness direction between the first electrode EL1 and the second electrode EL2. The light emitting structures OL-B1, OL-B2, and OL-B3 each may include an emission layer EML (FIG. 7) and a hole transport region HTR and an electron transport region ETR disposed with the emission layer EML (FIG. 7) located therebetween.

For example, the light emitting device ED-BT included in the display apparatus DD-TD of an embodiment may be a light emitting device having a tandem structure and including a plurality of emission layers.

In an embodiment illustrated in FIG. 8, all light (e.g., light beams) respectively emitted from the light emitting structures OL-B1, OL-B2, and OL-B3 may be blue light. However, the embodiment of the present disclosure is not limited thereto, and the light (e.g., light beams) respectively emitted from the light emitting structures OL-B1, OL-B2, and OL-B3 may have wavelength ranges different from each other. For example, the light emitting device ED-BT including the plurality of light emitting structures OL-B1, OL-B2, and OL-B3 which emit light (e.g., light beams) having wavelength ranges different from each other may be to emit white light.

Charge generation layers CGL1 and CGL2 may be respectively disposed between two of the neighboring light emitting structures OL-B1, OL-B2, and OL-B3. The charge generation layers CGL1 and CGL2 may include a p-type or kind charge generation layer (e.g., a P-charge generation layer) and/or an n-type or kind charge generation layer (e.g., an N-charge generation layer).

At least one from among the light emitting structures OL-B1, OL-B2, and OL-B3 included in the display apparatus DD-TD of an embodiment may contain the above-described fused polycyclic compound of an embodiment. For example, at least one from among the plurality of emission layers included in the light emitting device ED-BT may include the fused polycyclic compound of an embodiment.

FIG. 9 is a cross-sectional view illustrating a display apparatus according to an embodiment of the present disclosure; and FIG. 10 is a cross-sectional view illustrating a display apparatus according to an embodiment of the present disclosure.

Referring to FIG. 9, the display apparatus DD-b according to an embodiment may include light emitting devices ED-1, ED-2, and ED-3 in which two emission layers are stacked. Compared with the display apparatus DD of an embodiment illustrated in FIG. 2, an embodiment illustrated in FIG. 9 has a difference in that the first to third light emitting devices ED-1, ED-2, and ED-3 each include two emission layers stacked in the thickness direction. In each of the first to third light emitting devices ED-1, ED-2, and ED-3, the two emission layers may be to emit light in substantially the same wavelength region.

The first light emitting device ED-1 may include a first red emission layer EML-R1 and a second red emission layer EML-R2. The second light emitting device ED-2 may include a first green emission layer EML-G1 and a second green emission layer EML-G2. In some embodiments, the third light emitting device ED-3 may include a first blue emission layer EML-B1 and a second blue emission layer EML-B2. An emission auxiliary part OG may be disposed between the first red emission layer EML-R1 and the second red emission layer EML-R2, between the first green emission layer EML-G1 and the second green emission layer EML-G2, and between the first blue emission layer EML-B1 and the second blue emission layer EML-B2.

The emission auxiliary part OG may include a single layer or a multilayer. The emission auxiliary part OG may include a charge generation layer. More specifically, the emission auxiliary part OG may include an electron transport region, a charge generation layer, and a hole transport region that are sequentially stacked. The emission auxiliary part OG may be provided as a common layer in the whole of the first to third light emitting devices ED-1, ED-2, and ED-3. However, the embodiment of the present disclosure is not limited thereto, and the emission auxiliary part OG may be provided by being patterned within the openings OH defined in the pixel defining film PDL.

The first red emission layer EML-R1, the first green emission layer EML-G1, and the first blue emission layer EML-B1 may be disposed between the electron transport region ETR and the emission auxiliary part OG. The second red emission layer EML-R2, the second green emission layer EML-G2, and the second blue emission layer EML-B2 may be disposed between the emission auxiliary part OG and the hole transport region HTR.

For example, the first light emitting device ED-1 may include the first electrode EL1, the hole transport region HTR, the second red emission layer EML-R2, the emission auxiliary part OG, the first red emission layer EML-R1, the electron transport region ETR, and the second electrode EL2 that are sequentially stacked in the stated order. The second light emitting device ED-2 may include the first electrode EL1, the hole transport region HTR, the second green emission layer EML-G2, the emission auxiliary part OG, the first green emission layer EML-G1, the electron transport region ETR, and the second electrode EL2 that are sequentially stacked in the stated order. The third light emitting device ED-3 may include the first electrode EL1, the hole transport region HTR, the second blue emission layer EML-B2, the emission auxiliary part OG, the first blue emission layer EML-B1, the electron transport region ETR, and the second electrode EL2 that are sequentially stacked in the stated order.

In some embodiments, an optical auxiliary layer PL may be disposed on the display device layer DP-ED. The optical auxiliary layer PL may include a polarizing layer. The optical auxiliary layer PL may be disposed on the display panel DP and control reflection of external light at the display panel DP. Unlike the configuration illustrated, the optical auxiliary layer PL in the display apparatus according to an embodiment may not be provided.

At least one emission layer included in the display apparatus DD-b of an embodiment illustrated in FIG. 9 may include the above-described fused polycyclic compound of an embodiment. For example, in an embodiment, at least one of the first blue emission layer EML-B1 or the second blue emission layer EML-B2 may include the fused polycyclic compound of an embodiment.

Unlike FIGS. 8 and 9, FIG. 10 illustrates that a display apparatus DD-c includes four light emitting structures OL-B1, OL-B2, OL-B3, and OL-C1. A light emitting device ED-CT may include a first electrode EL1 and a second electrode EL2 which face each other, and first to fourth light emitting structures OL-B1, OL-B2, OL-B3, and OL-C1 that are sequentially stacked in the thickness direction between the first electrode EL1 and the second electrode EL2. Charge generation layers CGL1, CGL2, and CGL3 may be disposed between the first to fourth light emitting structures OL-B1, OL-B2, OL-B3, and OL-C1. Among the four light emitting structures, the first to third light emitting structures OL-B1, OL-B2, and OL-B3 may be to emit blue light, and the fourth light emitting structure OL-C1 may be to emit green light. However, the embodiment of the present disclosure is not limited thereto, and the first to fourth light emitting structures OL-B1, OL-B2, OL-B3, and OL-C1 may be to emit light (e.g., light beams) in different wavelength regions.

The charge generation layers CGL1, CGL2, and CGL3 disposed between adjacent light emitting structures OL-B1, OL-B2, OL-B3, and OL-C1 may include a p-type or kind charge generation layer and/or an n-type or kind charge generation layer.

At least one from among the light emitting structures OL-B1, OL-B2, OL-B3, and OL-C1 included in the display apparatus DD-c of an embodiment may contain the above-described fused polycyclic compound of an embodiment. For example, in an embodiment, at least one from among the first to third light emitting structures OL-B1, OL-B2, and OL-B3 may include the described-above fused polycyclic compound of an embodiment.

Hereinafter, with reference to Examples and Comparative Examples, a condensed polycyclic according to an embodiment of the present disclosure and a luminescence device of an embodiment of the present disclosure will be described in more detail. In some embodiments, Examples described are only illustrations to assist the understanding of the present disclosure, and the scope of the present disclosure is not limited thereto.

### EXAMPLES

### 1. Synthesis of Fused Polycyclic Compound

First, a synthetic method of the fused polycyclic compound according to the present embodiment will be described by illustrating synthetic methods of Compounds 1, 12, 29, 36, 41, 71, 79, and 121. In addition, the synthetic methods of the fused polycyclic compounds as described below are only examples, and the synthetic method of the fused polycyclic compound according to an embodiment of the present disclosure is not limited to the following examples.

### (1) Synthesis of Compound 1

Fused Polycyclic Compound 1 according to an example may be synthesized, for example, by the reaction below:

### Synthesis of Intermediate Compound 1-a

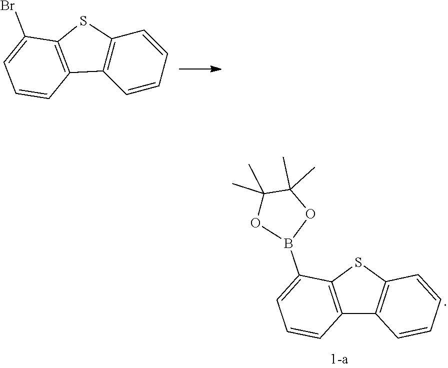 [SMILES: Brc1cccc2c1sc1ccccc12 ; CC1(C)OB(c2cccc3c2sc2ccccc23)OC1(C)C]

In an argon atmosphere, to a 2 L-flask, compound 4-bromodibenzo[b,d]thiophene (20 g, 76 mmol), bis(pinacolato)diboron (28.9 g, 114 mmol), potassium acetate (14.9 g, 152 mmol), and bis(triphenylphosphine)palladium(II) dichloride (2.7 g, 3.8 mmol) were added and dissolved in 700 mL of dioxane, and then the reaction solution was stirred at about 100° C. for about 12 hours. After the reaction solution was cooled, the solvent was removed under reduced pressure, and the reaction solution was extracted by adding water (500 mL) and ethyl acetate (500 mL) to collect organic layers, and the organic layers were dried over MgSO₄ and then filtered. In the filtrate, the solvent was removed under reduced pressure to obtain a solid. The solid thus obtained was filtered by silica gel pad utilizing CH₂C1₂ and hexane as the eluent, and recrystallized with ethanol to obtain Intermediate Compound 1-a (white solid, 18.9 g, 60.8 mmol, 80%). The obtained compound was identified as Intermediate Compound 1-a through ESI-LCMS.

ESI-LCMS: [M]⁺: C₁₈H₁₉BO₂S. 310.12

### Synthesis of Intermediate Compound 1-b

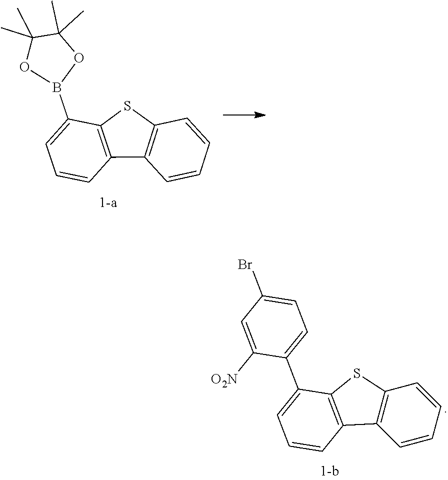 [SMILES: CC1(C)OB(c2cccc3c2sc2ccccc23)OC1(C)C ; O=[N+]([O-])c1cc(Br)ccc1-c1cccc2c1sc1ccccc12]

In an argon atmosphere, to a 1 L-flask, Intermediate Compound 1-a (19 g, 61 mmol), 1,4-dibromo-2-nitrobenzene (19 g, 67 mmol), Pd(PPh₃)₄ (3.5 g, 3.1 mmol), and potassium carbonate (25.3 g, 183 mmol) were added and dissolved in 250 mL of toluene, 50 mL of ethanol, and 100 mL of H₂O, and then the reaction solution was stirred at about 100° C. for about 12 hours. After being cooled, the reaction solution was extracted by adding water (500 mL) and ethyl acetate (300 mL) to collect organic layers, and the organic layers were dried over MgSO₄ and then filtered. In the filtrate, the solvent was removed under reduced pressure to obtain a solid. The solid thus obtained was purified and separated by silica gel column chromatography utilizing CH₂Cl₂ and hexane as the eluent to obtain Intermediate Compound 1-b (white solid, 17 g, 44 mmol, 72%). The obtained compound was identified as Intermediate Compound 1-b through ESI-LCMS.

ESI-LCMS: [M]⁺: C₁₈H₁₀BrNO₂S. 382.96

### Synthesis of Intermediate Compound 1-c

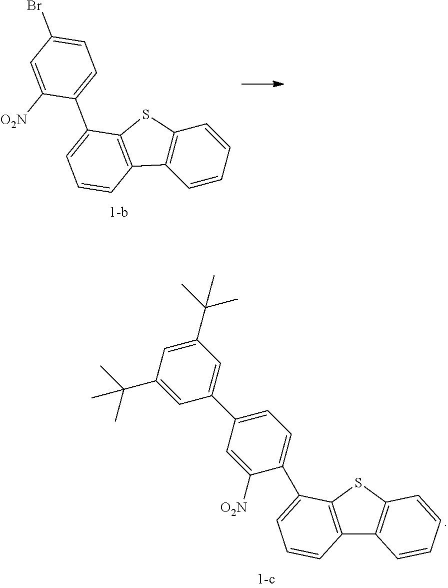 [SMILES: O=[N+]([O-])c1cc(Br)ccc1-c1cccc2c1sc1ccccc12 ; CC(C)(C)c1cc(-c2ccc(-c3cccc4c3sc3ccccc34)c([N+](=O)[O-])c2)cc(C(C)(C)C)c1]

In an argon atmosphere, to a 1 L-flask, Intermediate Compound 1-b (17 g, 44 mmol), (3,5-di-tert-butylphenyl)boronic acid (11 g, 48 mmol), Pd(PPh₃)₄ (2.5 g, 2.2 mmol), and potassium carbonate (18.2 g, 132 mmol) were added and dissolved in 200 mL of toluene, 40 mL of ethanol, and 80 mL of H₂O, and then the reaction solution was stirred at about 100° C. for about 12 hours. After being cooled, the reaction solution was extracted by adding water (300 mL) and ethyl acetate (200 mL) to collect organic layers, and the organic layers were dried over MgSO₄ and then filtered. In the filtrate, the solvent was removed under reduced pressure to obtain a solid. The solid thus obtained was purified and separated by silica gel column chromatography utilizing CH₂Cl₂ and hexane as the eluent to obtain Intermediate Compound 1-c (yellow solid, 15 g, 30 mmol, 68%). The obtained compound was identified as Intermediate Compound 1-c through ESI-LCMS.

ESI-LCMS: [M]⁺: C₃₂H₃₁NO₂S. 493.21

### Synthesis of Intermediate Compound 1-d

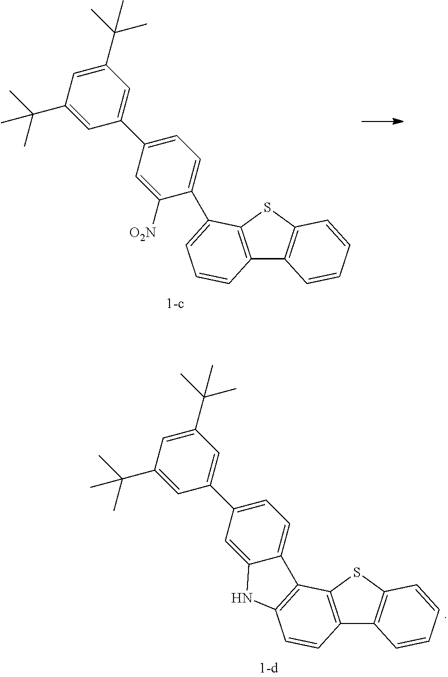 [SMILES: CC(C)(C)c1cc(-c2ccc(-c3cccc4c3sc3ccccc34)c([N+](=O)[O-])c2)cc(C(C)(C)C)c1 ; CC(C)(C)c1cc(-c2ccc3c(c2)[nH]c2ccc4c5ccccc5sc4c23)cc(C(C)(C)C)c1]

In an argon atmosphere, to a 500 mL-flask, Intermediate Compound 1-c (15 g, 30 mmol) and PPh₃ (17 g, 66 mmol) were added and dissolved in 125 mL of o-dichlorobenzene, and then the reaction solution was stirred at about 200° C. for about 12 hours. After the reaction solution was cooled, the solvent was removed under reduced pressure to obtain a solid. The solid thus obtained was filtered by silica gel pad utilizing CH₂Cl₂ and hexane as the eluent, and recrystallized with CH₂Cl₂ and hexane to obtain Intermediate Compound 1-d (white solid, 12 g, 26 mmol, 85%). The obtained compound was identified as Intermediate Compound 1-d through ESI-LCMS.

ESI-LCMS: [M]⁺: C₃₂H₃₁NS. 461.22.

### Synthesis of Intermediate Compound 1-e

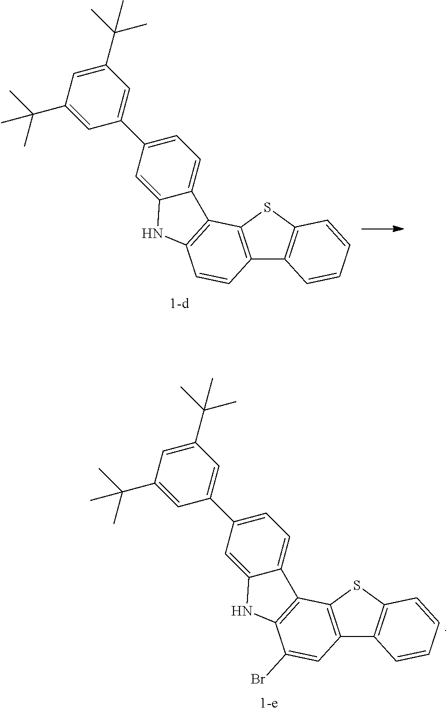 [SMILES: CC(C)(C)c1cc(-c2ccc3c(c2)[nH]c2ccc4c5ccccc5sc4c23)cc(C(C)(C)C)c1 ; CC(C)(C)c1cc(-c2ccc3c(c2)[nH]c2c(Br)cc4c5ccccc5sc4c23)cc(C(C)(C)C)c1]

In an argon atmosphere, to a dried 500 mL-flask, Intermediate Compound 1-d (12 g, 26 mmol), and Fe(OTf)₃ (0.26 g, 0.52 mmol) were added and dissolved in 250 mL of toluene, and then light was blocked. While the reaction solution was stirred, N-bromosuccinimide (5.1 g, 29 mmol) was added dropwise thereto, and then the resulting mixture was heated to 90° C. and then stirred for about 12 hours. After the reaction solution was cooled, the solvent was removed at under reduced pressure to obtain a solid. The solid thus obtained was filtered by silica gel pad utilizing CH₂Cl₂ and hexane as the eluent, and recrystallized with CH₂Cl₂ and hexane to obtain Intermediate Compound 1-e (white solid, 11 g, 21 mmol, 79%). The obtained compound was identified as Intermediate Compound 1-e through ESI-LCMS.

ESI-LCMS: [M]⁺: C₃₂H₃₀BrNS. 541.13.

### Synthesis of Intermediate Compound 1-f

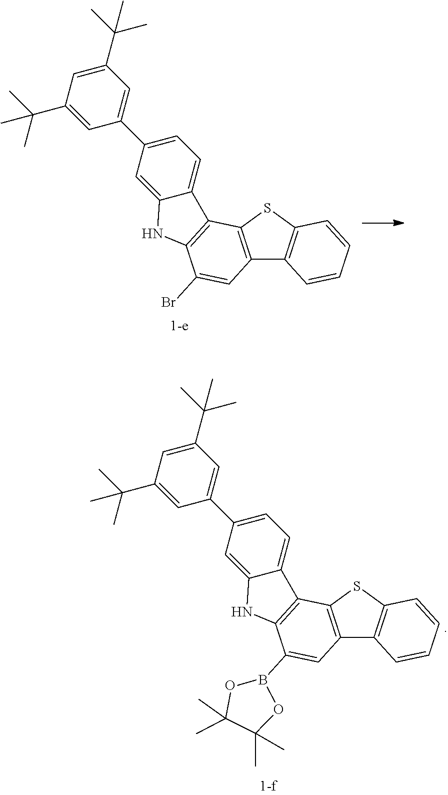 [SMILES: CC(C)(C)c1cc(-c2ccc3c(c2)[nH]c2c(Br)cc4c5ccccc5sc4c23)cc(C(C)(C)C)c1 ; CC(C)(C)c1cc(-c2ccc3c(c2)[nH]c2c(B4OC(C)(C)C(C)(C)O4)cc4c5ccccc5sc4c23)cc(C(C)(C)C)c1]

In an argon atmosphere, to a 500 mL-flask, Intermediate Compound 1-e (11 g, 21 mmol), bis(pinacolato)diboron (8.0 g, 32 mmol), potassium acetate (4.1 g, 42 mmol), and bis(triphenylphosphine)palladium(II) dichloride (0.74 g, 1.1 mmol) were added and dissolved in 200 mL of dioxane, and then the reaction solution was stirred at about 100° C. for about 12 hours. After the reaction solution was cooled, the solvent was removed under reduced pressure, and the reaction solution was extracted by adding water (300 mL) and ethyl acetate (300 mL) to collect organic layers, and the organic layers were dried over MgSO₄ and then filtered. In the filtrate, the solvent was removed under reduced pressure to obtain a solid. The solid thus obtained was filtered by silica gel pad utilizing CH₂Cl₂ and hexane as the eluent, and recrystallized with ethanol to obtain Intermediate Compound 1-f (white solid, 11 g, 18 mmol, 86%). The obtained compound was identified as Intermediate Compound 1-f through ESI-LCMS.

ESI-LCMS: [M]⁺: C₃₈H₄₂BNO₂S. 587.30

### Synthesis of Intermediate Compound 1-g

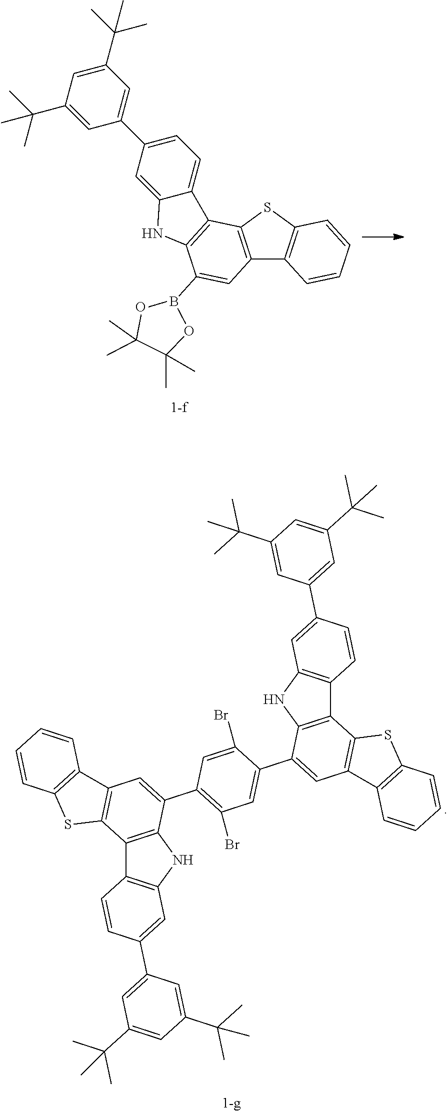 [SMILES: CC(C)(C)c1cc(-c2ccc3c(c2)[nH]c2c(B4OC(C)(C)C(C)(C)O4)cc4c5ccccc5sc4c23)cc(C(C)(C)C)c1 ; CC(C)(C)c1cc(-c2ccc3c(c2)[nH]c2c(-c4cc(Br)c(-c5cc6c7ccccc7sc6c6c5[nH]c5cc(-c7cc(C(C)(C)C)cc(C(C)(C)C)c7)ccc56)cc4Br)cc4c5ccccc5sc4c23)cc(C(C)(C)C)c1]

In an argon atmosphere, to a 500 mL-flask, Intermediate Compound 1-f (11 g, 18 mmol), 1,4-dibromo-2,5-diiodobenzene (3.5 g, 7.2 mmol), Pd(PPh₃)₄ (0.39 g, 0.34 mmol), and sodium carbonate (4.6 g, 34 mmol) were added and dissolved in 200 mL of toluene, 40 mL of DMSO, and 12 mL of H₂O, and then the reaction solution was stirred at about 90° C. for about 24 hours. After the reaction solution was cooled, toluene was removed under reduced pressure, and then water (100 mL) was added thereto to filter out a solid. Then, the solid was recrystallized by utilizing CH₂Cl₂ and methanol to obtain Intermediate Compound 1-g (white solid, 7.8 g, 6.8 mmol, 94%). The obtained compound was identified as Intermediate Compound 1-g through ESI-LCMS.

ESI-LCMS: [M]⁺: C₇₀H₆₂Br₂N₂S₂. 1152.27

### Synthesis of Compound 1

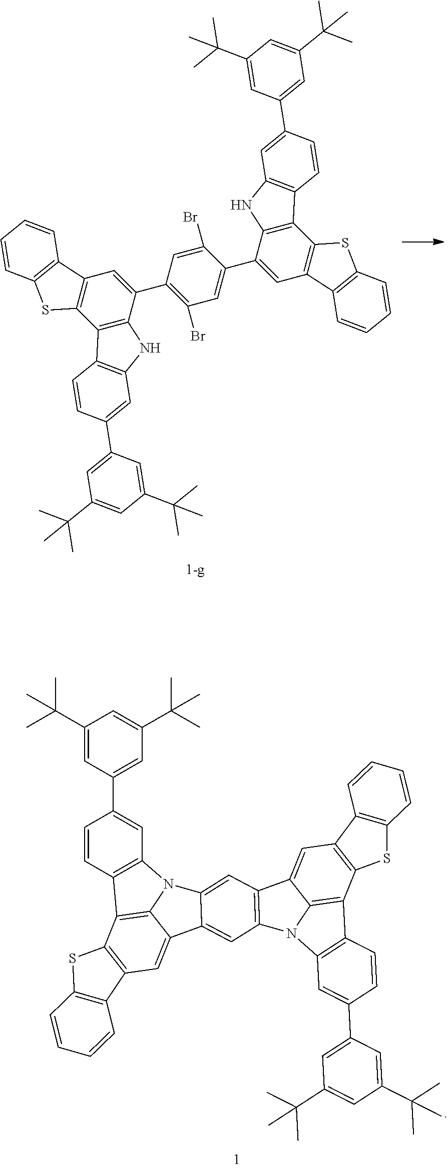 [SMILES: CC(C)(C)c1cc(-c2ccc3c(c2)[nH]c2c(-c4cc(Br)c(-c5cc6c7ccccc7sc6c6c5[nH]c5cc(-c7cc(C(C)(C)C)cc(C(C)(C)C)c7)ccc56)cc4Br)cc4c5ccccc5sc4c23)cc(C(C)(C)C)c1 ; CC(C)(C)c1cc(-c2ccc3c4c5sc6ccccc6c5cc5c6cc7c(cc6n(c3c2)c54)c2cc3c4ccccc4sc3c3c4ccc(-c5cc(C(C)(C)C)cc(C(C)(C)C)c5)cc4n7c23)cc(C(C)(C)C)c1]

In an argon atmosphere, to a 500 mL-flask, Intermediate Compound 1-g (7.8 g, 6.8 mmol), CuI (0.65 g, 3.4 mmol), potassium acetate (2.8 g, 20 mmol), and L-proline (0.39 g, 3.4 mmol) were added and dissolved in 125 mL of DMSO, and then the reaction solution was stirred at about 150° C. for about 6 hours. The reaction solution was cooled and then filtered to obtain a yellow solid, and then the yellow solid was washed with water and ethanol. The solid thus obtained was filtered by silica gel pad utilizing CH₂Cl₂ as the eluent, and recrystallized with CH₂Cl₂ and hexane to obtain Compound 1 (yellow solid, 2.8 g, 2.9 mmol, 42%). The obtained compound was identified as Compound 1 through ESI-LCMS.

ESI-LCMS: [M]*: C₇₀H₆₀N₂S₂. 992.42

### (2) Synthesis of Compound 12

Fused Polycyclic Compound 12 according to an example may be synthesized by, for example, the reaction below:

### Synthesis of Intermediate Compound 12-a

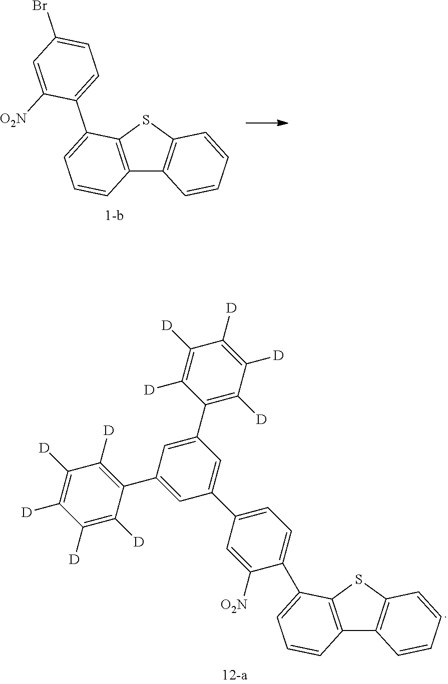 [SMILES: O=[N+]([O-])c1cc(Br)ccc1-c1cccc2c1sc1ccccc12 ; [2H]c1c([2H])c([2H])c(-c2cc(-c3ccc(-c4cccc5c4sc4ccccc45)c([N+](=O)[O-])c3)cc(-c3c([2H])c([2H])c([2H])c([2H])c3[2H])c2)c([2H])c1[2H]]

In an argon atmosphere, to a 1 L-flask, Intermediate Compound 1-b (20 g, 52 mmol), ([1,1′:3′,1″-terphenyl]-5′-yl-2,2″,3,3″,4,4″,5,5″,6,6″-d10)boronic acid (16.3 g, 57 mmol), Pd(PPh₃)₄ (3 g, 2.6 mmol), and potassium carbonate (21.6 g, 156 mmol) were added and dissolved in 250 mL of toluene, 50 mL of ethanol, and 100 mL of H₂O, and then the reaction solution was stirred at about 100° C. for about 12 hours. After cooling, the reaction solution was extracted by adding water (300 mL) and ethyl acetate (200 mL) to collect organic layers, and the organic layers were dried over MgSO₄ and then filtered. In the filtrate, the solvent was removed under reduced pressure to obtain a solid. The solid thus obtained was purified and separated by silica gel column chromatography utilizing CH₂Cl₂ and hexane as the eluent to obtain Intermediate Compound 12-a (yellow solid, 17.5 g, 32.2 mmol, 62%). The obtained compound was identified as Intermediate Compound 12-a through ESI-LCMS.

ESI-LCMS: [M]*: C₃₆H₁₃D₁₀NO₂S. 543.21

### Synthesis of Intermediate Compound 12-b

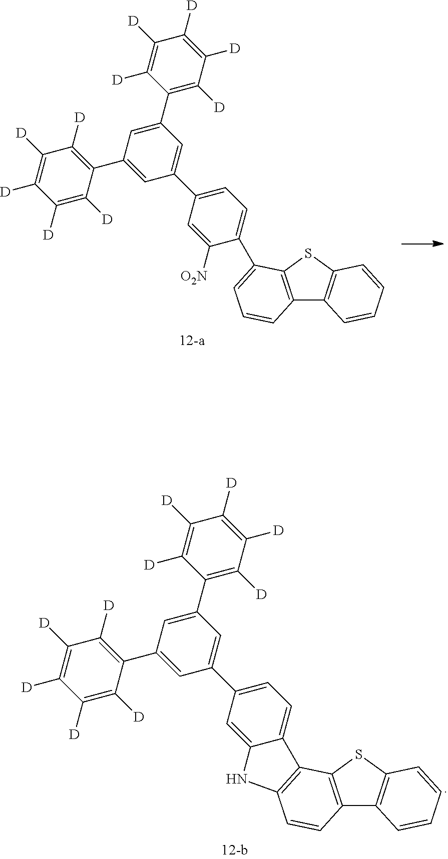 [SMILES: [2H]c1c([2H])c([2H])c(-c2cc(-c3ccc(-c4cccc5c4sc4ccccc45)c([N+](=O)[O-])c3)cc(-c3c([2H])c([2H])c([2H])c([2H])c3[2H])c2)c([2H])c1[2H] ; [2H]c1c([2H])c([2H])c(-c2cc(-c3ccc4c(c3)[nH]c3ccc5c6ccccc6sc5c34)cc(-c3c([2H])c([2H])c([2H])c([2H])c3[2H])c2)c([2H])c1[2H]]

In an argon atmosphere, to a 500 mL-flask, Intermediate Compound 12-a (17.5 g, 32.2 mmol) and PPh₃ (18.6 g, 71 mmol) were added and dissolved in 150 mL of o-dichlorobenzene, and then the reaction solution was stirred at about 200° C. for about 12 hours. After the reaction solution was cooled, the solvent was removed under reduced pressure to obtain a solid. The solid thus obtained was filtered by silica gel pad utilizing CH₂Cl₂ and hexane as the eluent, and recrystallized with CH₂Cl₂ and hexane to obtain Intermediate Compound 12-b (white solid, 13.5 g, 26.4 mmol, 82%). The obtained compound was identified as Intermediate Compound 12-b through ESI-LCMS.

ESI-LCMS: [M]⁺: C₃₆H₁₃D₁₀NS. 511.22.

### Synthesis of Intermediate Compound 12-c

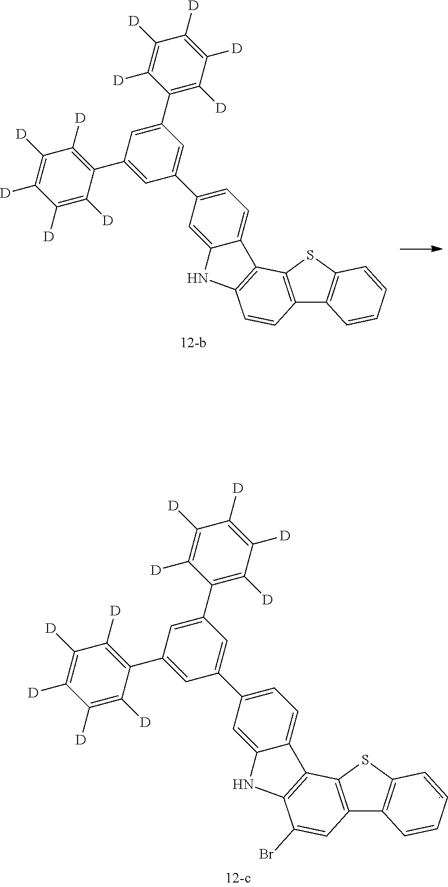 [SMILES: [2H]c1c([2H])c([2H])c(-c2cc(-c3ccc4c(c3)[nH]c3ccc5c6ccccc6sc5c34)cc(-c3c([2H])c([2H])c([2H])c([2H])c3[2H])c2)c([2H])c1[2H] ; [2H]c1c([2H])c([2H])c(-c2cc(-c3ccc4c(c3)[nH]c3c(Br)cc5c6ccccc6sc5c34)cc(-c3c([2H])c([2H])c([2H])c([2H])c3[2H])c2)c([2H])c1[2H]]

In an argon atmosphere, to a dried 500 mL-flask, Intermediate Compound 12-b (13.5 g, 26.4 mmol), and Fe(OTf)₃ (0.27 g, 0.53 mmol) were added and dissolved in 250 mL of toluene, and then light was blocked. While the reaction solution was stirred, N-bromosuccinimide (5.1 g, 29 mmol) was added dropwise thereto, and then the resulting mixture was heated to 90° C. and then stirred for about 12 hours. After the reaction solution was cooled, the solvent was removed at under reduced pressure to obtain a solid. The solid thus obtained was filtered by silica gel pad utilizing CH₂Cl₂ and hexane as the eluent, and recrystallized with CH₂Cl₂ and hexane to obtain Intermediate Compound 12-c (white solid, 13.3 g, 22.4 mmol, 85%). The obtained compound was identified as Intermediate Compound 12-c through ESI-LCMS.

ESI-LCMS: [M]⁺: C₃₆H₁₂D₁₀BrNS. 589.13.

### Synthesis of Intermediate Compound 12-d

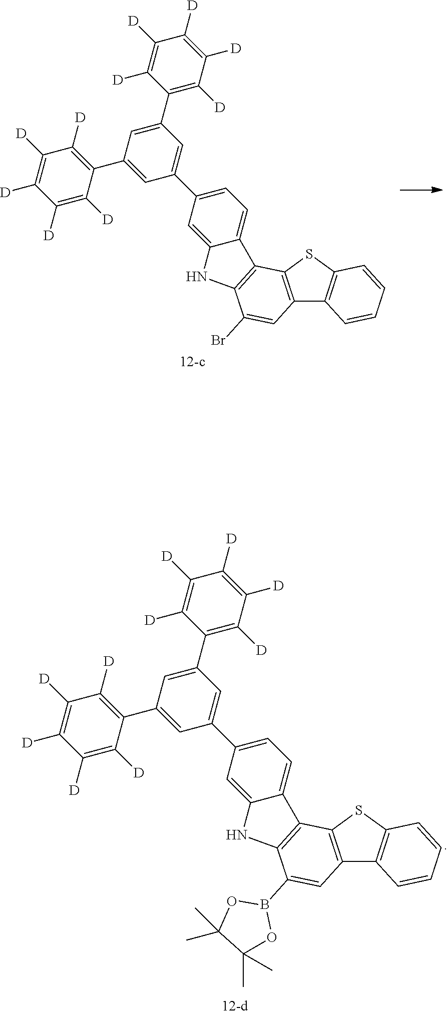 [SMILES: [2H]c1c([2H])c([2H])c(-c2cc(-c3ccc4c(c3)[nH]c3c(Br)cc5c6ccccc6sc5c34)cc(-c3c([2H])c([2H])c([2H])c([2H])c3[2H])c2)c([2H])c1[2H] ; [2H]c1c([2H])c([2H])c(-c2cc(-c3ccc4c(c3)[nH]c3c(B5OC(C)(C)C(C)(C)O5)cc5c6ccccc6sc5c34)cc(-c3c([2H])c([2H])c([2H])c([2H])c3[2H])c2)c([2H])c1[2H]]

In an argon atmosphere, to a 500 mL-flask, Intermediate Compound 12-c (13.3 g, 22.4 mmol), bis(pinacolato)diboron (7.4 g, 29 mmol), potassium acetate (4.4 g, 45 mmol), and bis(triphenylphosphine)palladium(II) dichloride (0.74 g, 1.1 mmol) were added and dissolved in 200 mL of dioxane, and then the reaction solution was stirred at about 100° C. for about 12 hours. After the reaction solution was cooled, the solvent was removed under reduced pressure, and the reaction solution was extracted by adding water (300 mL) and ethyl acetate (300 mL) to collect organic layers, and the organic layers were dried over MgSO₄ and then filtered. In the filtrate, the solvent was removed under reduced pressure to obtain a solid. The solid thus obtained was filtered by silica gel pad utilizing CH₂Cl₂ and hexane as the eluent, and recrystallized with ethanol to obtain Intermediate Compound 12-d (white solid, 11.4 g, 18 mmol, 80%). The obtained compound was identified as Intermediate Compound 12-d through ESI-LCMS.

ESI-LCMS: [M]⁺: C₄₂H₂₄D₁₀BNO₂S. 637.30

### Synthesis of Intermediate Compound 12-e

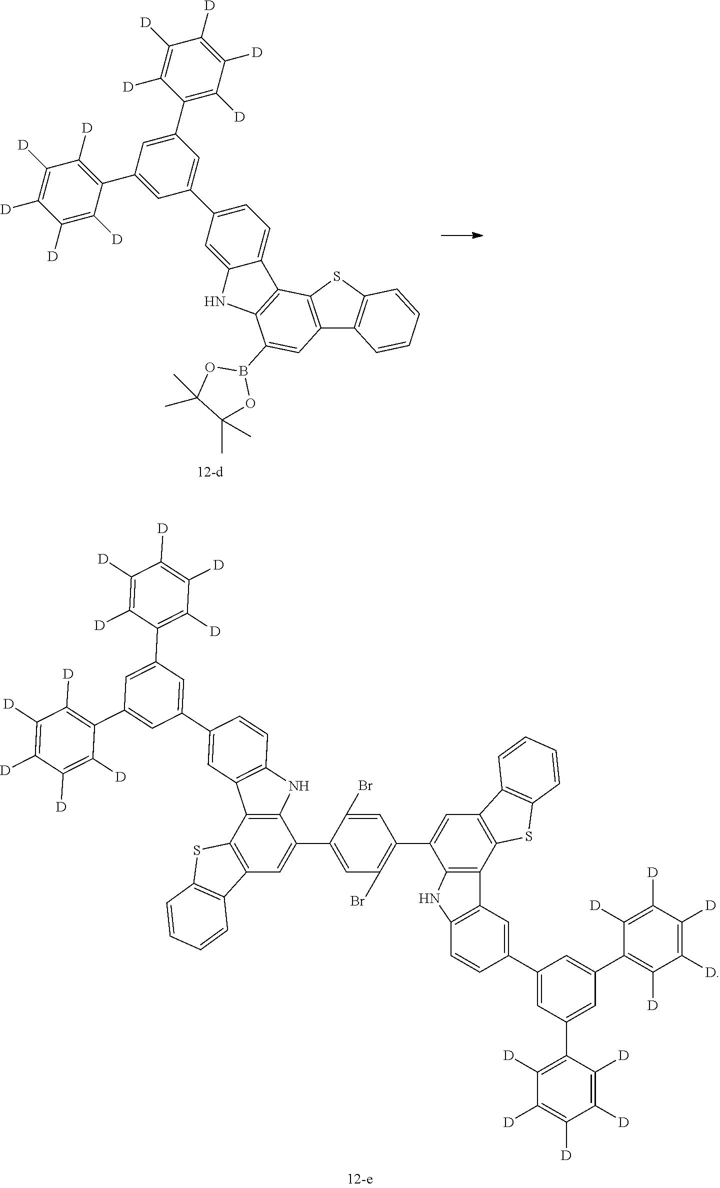

In an argon atmosphere, to a 500 mL-flask, Intermediate Compound 12-d (11.4 g, 18 mmol), 1,4-dibromo-2,5-diiodobenzene (3.5 g, 7.2 mmol), Pd(PPh₃)₄ (0.39 g, 0.34 mmol), and sodium carbonate (4.6 g, 34 mmol) were added and dissolved in 200 mL of toluene, 40 mL of DMSO, and 12 mL of H₂O, and then the reaction solution was stirred at about 90° C. for about 24 hours. After the reaction solution was cooled, toluene was removed under reduced pressure, and then water (100 mL) was added thereto to filter out a solid. Then, the solid was recrystallized by utilizing CH₂Cl₂ and methanol to obtain Intermediate Compound 12-e (white solid, 8.0 g, 6.4 mmol, 89%). The obtained compound was identified as Intermediate Compound 12-e through ESI-LCMS.

ESI-LCMS: [M]*: C₇₈H₂₆D₂₀Br₂N₂S₂. 1254.27

### Synthesis of Compound 12

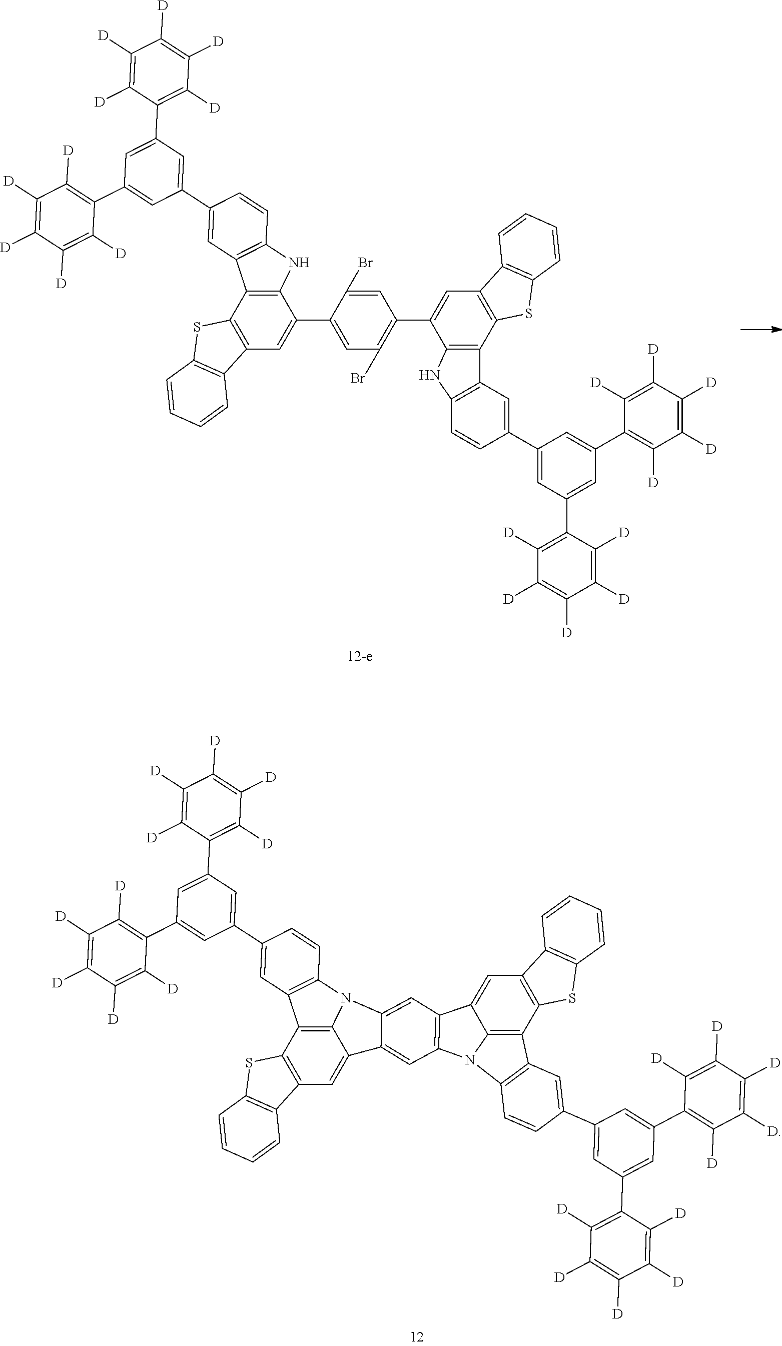

In an argon atmosphere, to a 500 mL-flask, Intermediate Compound 12-e (8.0 g, 6.4 mmol), CuI (0.61 g, 3.2 mmol), potassium acetate (2.7 g, 19.2 mmol), and L-proline (0.37 g, 3.2 mmol) were added and dissolved in 125 mL of DMSO, and then the reaction solution was stirred at about 150° C. for about 6 hours. The reaction solution was cooled and then filtered to obtain a yellow solid, and then the yellow solid was washed with water and ethanol. The solid thus obtained was filtered by silica gel pad utilizing CH₂Cl₂ as the eluent, and recrystallized with CH₂Cl₂ and hexane to obtain Compound 12 (yellow solid, 2.6 g, 2.4 mmol, 37%). The obtained compound was identified as Compound 12 through ESI-LCMS.

ESI-LCMS: [M]*: C₇₈H₂₄D₂₀N₂S₂. 1092.42

### (3) Synthesis of Compound 29

Fused Polycyclic Compound 29 according to an example may be synthesized by, for example, the reaction below:

### Synthesis of Intermediate Compound 29-a

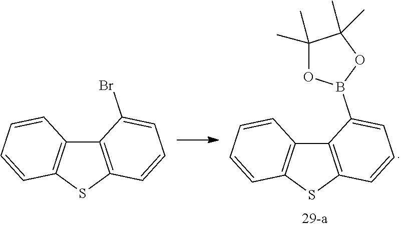 [SMILES: Brc1cccc2sc3ccccc3c12 ; CC1(C)OB(c2cccc3sc4ccccc4c23)OC1(C)C]

In an argon atmosphere, to a 2 L-flask, compound 1-bromodibenzo[b,d]thiophene (20 g, 76 mmol), bis(pinacolato)diboron (28.9 g, 114 mmol), potassium acetate (14.9 g, 152 mmol), and bis(triphenylphosphine)palladium(II) dichloride (2.7 g, 3.8 mmol) were added and dissolved in 700 mL of dioxane, and then the reaction solution was stirred at about 100° C. for about 12 hours. After the reaction solution was cooled, the solvent was removed under reduced pressure, and the reaction solution was extracted by adding water (500 mL) and ethyl acetate (500 mL) to collect organic layers, and the organic layers were dried over MgSO₄ and then filtered. In the filtrate, the solvent was removed under reduced pressure to obtain a solid. The solid thus obtained was filtered by silica gel pad utilizing CH₂Cl₂ and hexane as the eluent, and recrystallized with ethanol to obtain Intermediate Compound 29-a (white solid, 20.0 g, 64.6 mmol, 85%). The obtained compound was identified as Intermediate Compound 29-a through ESI-LCMS.

ESI-LCMS: [M]⁺: C₁₈H₁₉BO₂S. 310.12

### Synthesis of Intermediate Compound 29-b

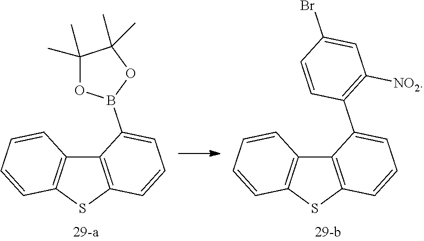 [SMILES: CC1(C)OB(c2cccc3sc4ccccc4c23)OC1(C)C ; O=[N+]([O-])c1cc(Br)ccc1-c1cccc2sc3ccccc3c12]

In an argon atmosphere, to a 1 L-flask, Intermediate Compound 29-a (20.0 g, 64.6 mmol), 1,4-dibromo-2-nitrobenzene (20.0 g, 71.1 mmol), Pd(PPh₃)₄ (3.73 g, 3.23 mmol), and potassium carbonate (26.8 g, 194 mmol) were added and dissolved in 250 mL of toluene, 50 mL of ethanol, and 100 mL of H₂O, and then the reaction solution was stirred at about 100° C. for about 12 hours. After being cooled, the reaction solution was extracted by adding water (500 mL) and ethyl acetate (300 mL) to collect organic layers, and the organic layers were dried over MgSO₄ and then filtered. In the filtrate, the solvent was removed under reduced pressure to obtain a solid. The solid thus obtained was purified and separated by silica gel column chromatography utilizing CH₂Cl₂ and hexane as the eluent to obtain Intermediate Compound 29-b (yellow solid, 17.4 g, 45.2 mmol, 70%). The obtained compound was identified as Intermediate Compound 29-b through ESI-LCMS.

ESI-LCMS: [M]⁺: C₁₈H₁₀BrNO₂S. 382.96

### Synthesis of Intermediate Compound 29-c

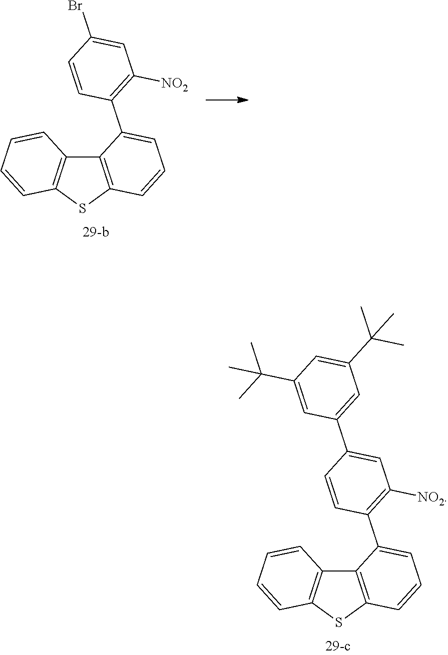 [SMILES: O=[N+]([O-])c1cc(Br)ccc1-c1cccc2sc3ccccc3c12 ; CC(C)(C)c1cc(-c2ccc(-c3cccc4sc5ccccc5c34)c([N+](=O)[O-])c2)cc(C(C)(C)C)c1]

In an argon atmosphere, to a 1 L-flask, Intermediate Compound 29-b (17.4 g, 45.2 mmol), (3,5-di-tert-butylphenyl)boronic acid (11.6 g, 49.7 mmol), Pd(PPh₃)₄ (2.61 g, 2.26 mmol), and potassium carbonate (18.7 g, 136 mmol) were added and dissolved in 200 mL of toluene, 40 mL of ethanol, and 80 mL of H₂O, and then the reaction solution was stirred at about 100° C. for about 12 hours. After being cooled, the reaction solution was extracted by adding water (300 mL) and ethyl acetate (200 mL) to collect organic layers, and the organic layers were dried over MgSO₄ and then filtered. In the filtrate, the solvent was removed under reduced pressure to obtain a solid. The solid thus obtained was purified and separated by silica gel column chromatography utilizing CH₂Cl₂ and hexane as the eluent to obtain Intermediate Compound 29-c (yellow solid, 17.2 g, 34.8 mmol, 77%). The obtained compound was identified as Intermediate Compound 29-c through ESI-LCMS.

ESI-LCMS: [M]⁺: C₃₂H₃₁NO₂S. 493.21

### Synthesis of Intermediate Compound 29-d

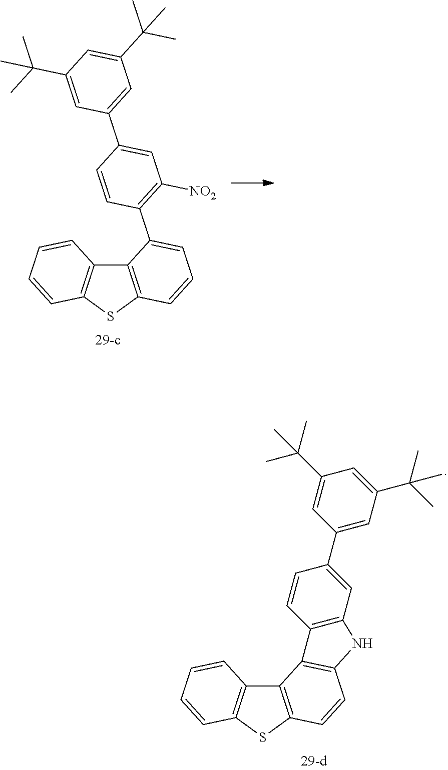 [SMILES: CC(C)(C)c1cc(-c2ccc(-c3cccc4sc5ccccc5c34)c([N+](=O)[O-])c2)cc(C(C)(C)C)c1 ; CC(C)(C)c1cc(-c2ccc3c(c2)[nH]c2ccc4sc5ccccc5c4c23)cc(C(C)(C)C)c1]

In an argon atmosphere, to a 500 mL-flask, Intermediate Compound 29-c (17.2 g, 34.8 mmol) and PPh₃ (20.1 g, 76.7 mmol) were added and dissolved in 125 mL of o-dichlorobenzene, and then the reaction solution was stirred at about 200° C. for about 12 hours. After the reaction solution was cooled, the solvent was removed under reduced pressure to obtain a solid. The solid thus obtained was filtered by silica gel pad utilizing CH₂Cl₂ and hexane as the eluent, and recrystallized with CH₂Cl₂ and hexane to obtain Intermediate Compound 29-d (white solid, 12.8 g, 27.8 mmol, 80%). The obtained compound was identified as Intermediate Compound 29-d through ESI-LCMS.

ESI-LCMS: [M]⁺: C₃₂H₃₁NS. 461.22.

### Synthesis of Intermediate Compound 29-e

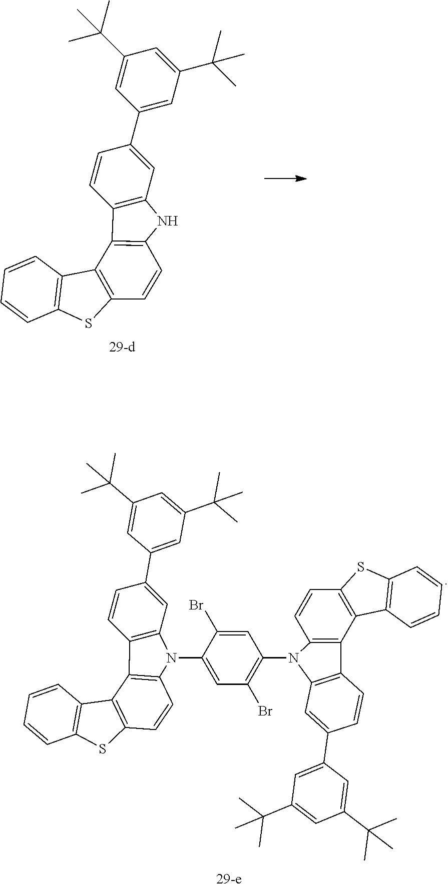 [SMILES: CC(C)(C)c1cc(-c2ccc3c(c2)[nH]c2ccc4sc5ccccc5c4c23)cc(C(C)(C)C)c1 ; CC(C)(C)c1cc(-c2ccc3c4c5c(ccc4n(-c4cc(Br)c(-n6c7cc(-c8cc(C(C)(C)C)cc(C(C)(C)C)c8)ccc7c7c8c(ccc76)sc6ccccc68)cc4Br)c3c2)sc2ccccc25)cc(C(C)(C)C)c1]

In an argon atmosphere, to a 500 mL-flask, Intermediate Compound 29-d (12.8 g, 27.8 mmol), 1,4-dibromo-2,5-difluorobenzene (3.0 g, 11.1 mmol), and K₃PO₄ (7.1 g, 33.3 mmol) were dissolved in 200 mL of DMF, and then the reaction solution was stirred at about 150° C. for about 12 hours. After the reaction solution was cooled, the solvent was removed under reduced pressure to obtain a solid. The solid thus obtained was dissolved in CH₂Cl₂ and subjected to celite pad filter, and the filtrate was filtered with silica gel pad. Then, the solid was recrystallized by utilizing CH₂Cl₂ and methanol to obtain Intermediate Compound 29-e (white solid, 10.5 g, 9.1 mmol, 82%). The obtained compound was identified as Intermediate Compound 29-e through ESI-LCMS.

ESI-LCMS: [M]⁺: C₇₀H₆₂Br₂N₂S₂. 1152.27

### Synthesis of Compound 29

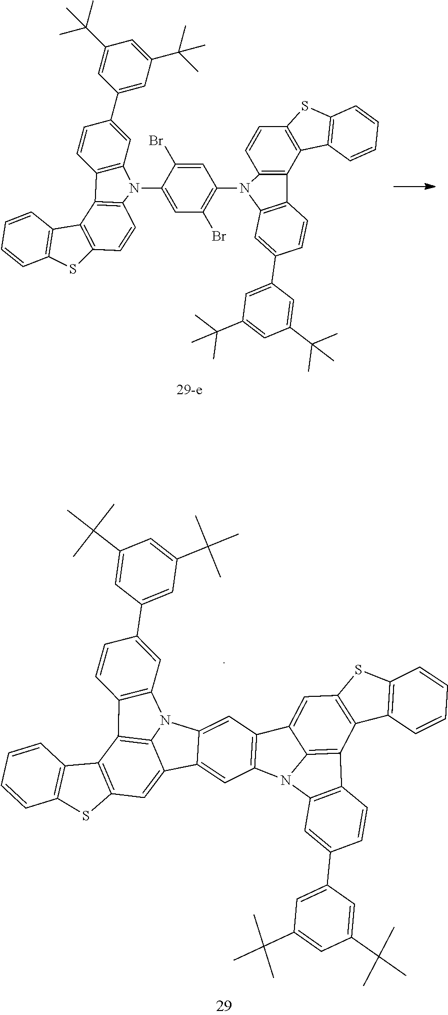 [SMILES: CC(C)(C)c1cc(-c2ccc3c4c5c(ccc4n(-c4cc(Br)c(-n6c7cc(-c8cc(C(C)(C)C)cc(C(C)(C)C)c8)ccc7c7c8c(ccc76)sc6ccccc68)cc4Br)c3c2)sc2ccccc25)cc(C(C)(C)C)c1 ; CC(C)(C)c1cc(-c2ccc3c4c5c(cc6c7cc8c(cc7n(c3c2)c64)c2cc3sc4ccccc4c3c3c4ccc(-c6cc(C(C)(C)C)cc(C(C)(C)C)c6)cc4n8c23)sc2ccccc25)cc(C(C)(C)C)c1]

In an argon atmosphere, to a 250 mL-flask, Intermediate Compound 29-e (10.5 g, 9.1 mmol), palladium(II) acetate (0.41 g, 1.82 mmol), potassium carbonate (12.6 g, 91 mmol), and triphenylphosphine (2.4 g, 9.1 mmol) were added and dissolved in 90 mL of DMAC, and then the reaction solution was stirred at about 180° C. for about 12 hours. The reaction solution was cooled and then filtered to obtain a yellow solid, and then the yellow solid was washed with water and ethanol. The solid thus obtained was subjected to flash column utilizing CH₂Cl₂ as the eluent, and recrystallized with CH₂Cl₂ and hexane to obtain Compound 29 (yellow solid, 1.45 g, 1.5 mmol, 16%). The obtained compound was identified as Compound 29 through ESI-LCMS.

ESI-LCMS: [M]*: C₇₀H₆₀N₂S₂. 992.42

### (4) Synthesis of Compound 36

Fused Polycyclic Compound 36 according to an example may be synthesized, for example, by the reaction below:

### Synthesis of Intermediate Compound 36-a

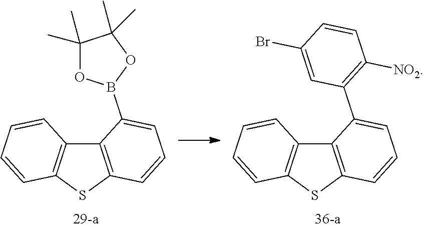 [SMILES: CC1(C)OB(c2cccc3sc4ccccc4c23)OC1(C)C ; O=[N+]([O-])c1ccc(Br)cc1-c1cccc2sc3ccccc3c12]

In an argon atmosphere, to a 1 L-flask, Intermediate Compound 29-a (20.0 g, 64.6 mmol), 2,4-dibromo-1-nitrobenzene (20.0 g, 71.1 mmol), Pd(PPh₃)₄ (3.73 g, 3.23 mmol), and potassium carbonate (26.8 g, 194 mmol) were added and dissolved in 250 mL of toluene, 50 mL of ethanol, and 100 mL of H₂O, and then the reaction solution was stirred at about 100° C. for about 12 hours. After being cooled, the reaction solution was extracted by adding water (500 mL) and ethyl acetate (300 mL) to collect organic layers, and the organic layers were dried over MgSO₄ and then filtered. In the filtrate, the solvent was removed under reduced pressure to obtain a solid. The solid thus obtained was purified and separated by silica gel column chromatography utilizing CH₂Cl₂ and hexane as the eluent to obtain Intermediate Compound 36-a (yellow solid, 19.4 g, 50.4 mmol, 78%). The obtained compound was identified as Intermediate Compound 36-a through ESI-LCMS.

ESI-LCMS: [M]⁺: C₁₈H₁₀BrNO₂S. 382.96

### Synthesis of Intermediate Compound 36-b

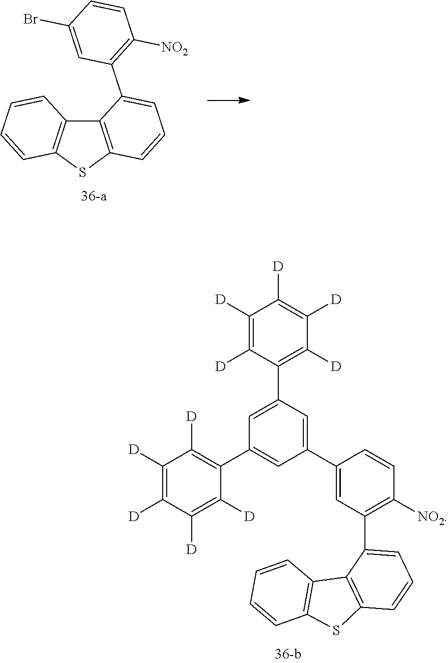 [SMILES: O=[N+]([O-])c1ccc(Br)cc1-c1cccc2sc3ccccc3c12 ; [2H]c1c([2H])c([2H])c(-c2cc(-c3ccc([N+](=O)[O-])c(-c4cccc5sc6ccccc6c45)c3)cc(-c3c([2H])c([2H])c([2H])c([2H])c3[2H])c2)c([2H])c1[2H]]

In an argon atmosphere, to a 1 L-flask, Intermediate Compound 36-a (19.4 g, 50.4 mmol), ([1,1′:3′,1″-terphenyl]-5′-yl-2,2″,3,3″,4,4″,5,5″,6,6″-d10)boronic acid (15.8 g, 55.4 mmol), Pd(PPh₃)₄ (2.91 g, 2.52 mmol), and potassium carbonate (20.9 g, 151 mmol) were added and dissolved in 300 mL of toluene, 60 mL of ethanol, and 120 mL of H₂O, and then the reaction solution was stirred at about 100° C. for about 12 hours. After being cooled, the reaction solution was extracted by adding water (300 mL) and ethyl acetate (200 mL) to collect organic layers, and the organic layers were dried over MgSO₄ and then filtered. In the filtrate, the solvent was removed under reduced pressure to obtain a solid. The solid thus obtained was purified and separated by silica gel column chromatography utilizing CH₂Cl₂ and hexane as the eluent to obtain Intermediate Compound 36-b (yellow solid, 19.7 g, 36.3 mmol, 72%). The obtained compound was identified as Intermediate Compound 36-b through ESI-LCMS.

ESI-LCMS: [M]⁺: C₃₆H₁₃D₁₀NO₂S. 543.21

### Synthesis of Intermediate Compound 36-c

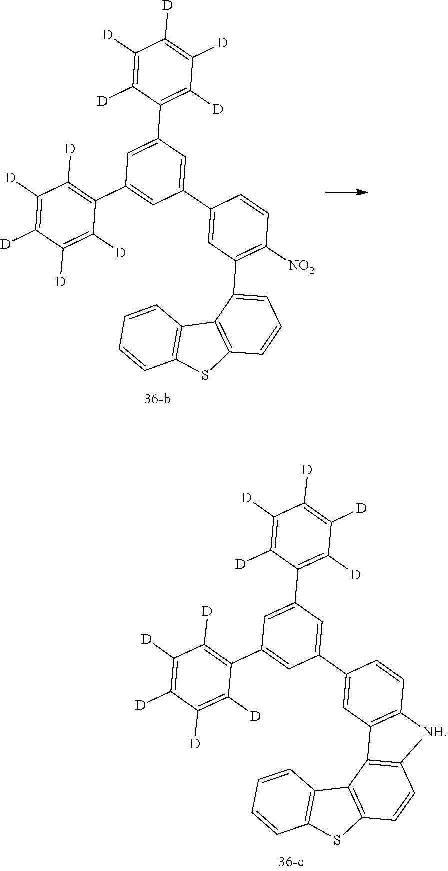 [SMILES: [2H]c1c([2H])c([2H])c(-c2cc(-c3ccc([N+](=O)[O-])c(-c4cccc5sc6ccccc6c45)c3)cc(-c3c([2H])c([2H])c([2H])c([2H])c3[2H])c2)c([2H])c1[2H] ; [2H]c1c([2H])c([2H])c(-c2cc(-c3ccc4[nH]c5ccc6sc7ccccc7c6c5c4c3)cc(-c3c([2H])c([2H])c([2H])c([2H])c3[2H])c2)c([2H])c1[2H]]

In an argon atmosphere, to a 500 mL-flask, Intermediate Compound 36-b (19.7 g, 36.3 mmol) and PPh₃ (21.0 g, 79.9 mmol) were added and dissolved in 125 mL of o-dichlorobenzene, and then the reaction solution was stirred at about 200° C. for about 12 hours. After the reaction solution was cooled, the solvent was removed under reduced pressure to obtain a solid. The solid thus obtained was filtered by silica gel pad utilizing CH₂Cl₂ and hexane as the eluent, and recrystallized with CH₂Cl₂ and hexane to obtain Intermediate Compound 36-c (white solid, 16.5 g, 32.3 mmol, 89%). The obtained compound was identified as Intermediate Compound 36-c through ESI-LCMS.

ESI-LCMS: [M]⁺: C₃₆H₁₃D₁₀NS. 511.22.

### Synthesis of Intermediate Compound 36-d

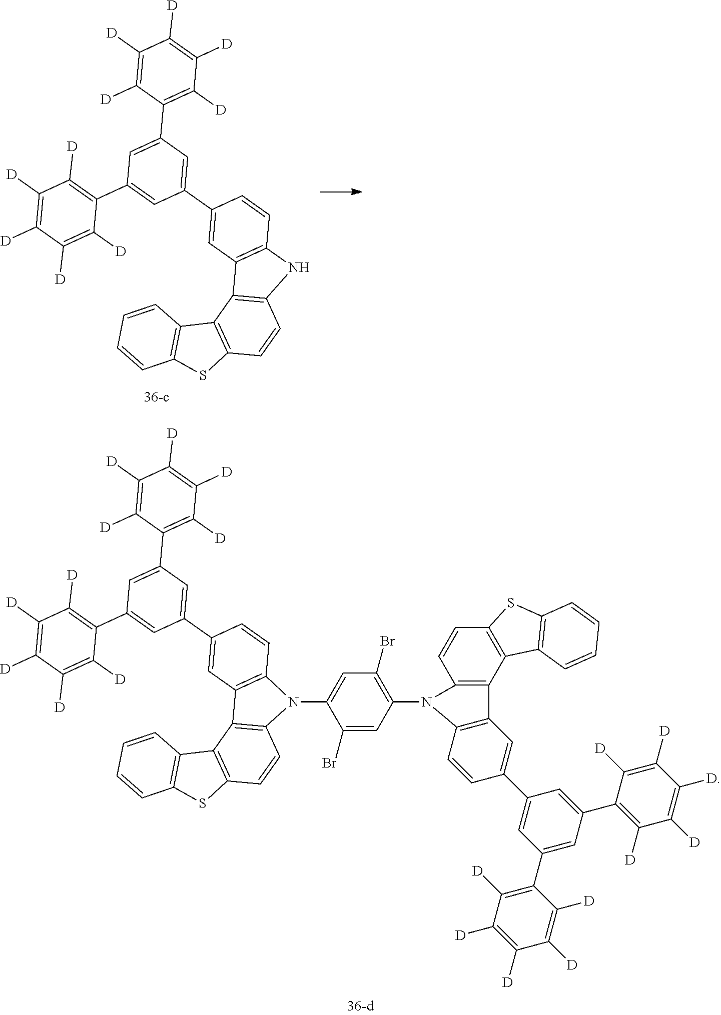

In an argon atmosphere, to a 500 mL-flask, Intermediate Compound 36-c (16.5 g, 32.3 mmol), 1,4-dibromo-2,5-difluorobenzene (3.5 g, 12.9 mmol), and K₃PO₄ (8.2 g, 38.7 mmol) were dissolved in 200 mL of DMF, and then the reaction solution was stirred at about 150° C. for about 12 hours. After the reaction solution was cooled, the solvent was removed under reduced pressure to obtain a solid. The solid thus obtained was dissolved in CH₂Cl₂ and subjected to celite pad filter, and the filtrate was filtered with silica gel pad. Then, the solid was recrystallized by utilizing CH₂Cl₂ and methanol to obtain Intermediate Compound 36-d (white solid, 13.0 g, 10.3 mmol, 80%). The obtained compound was identified as Intermediate Compound 36-d through ESI-LCMS.

ESI-LCMS: [M]⁺: C₇8H₂₆D₂₀Br₂N₂S₂. 1254.27

### Synthesis of Compound 36

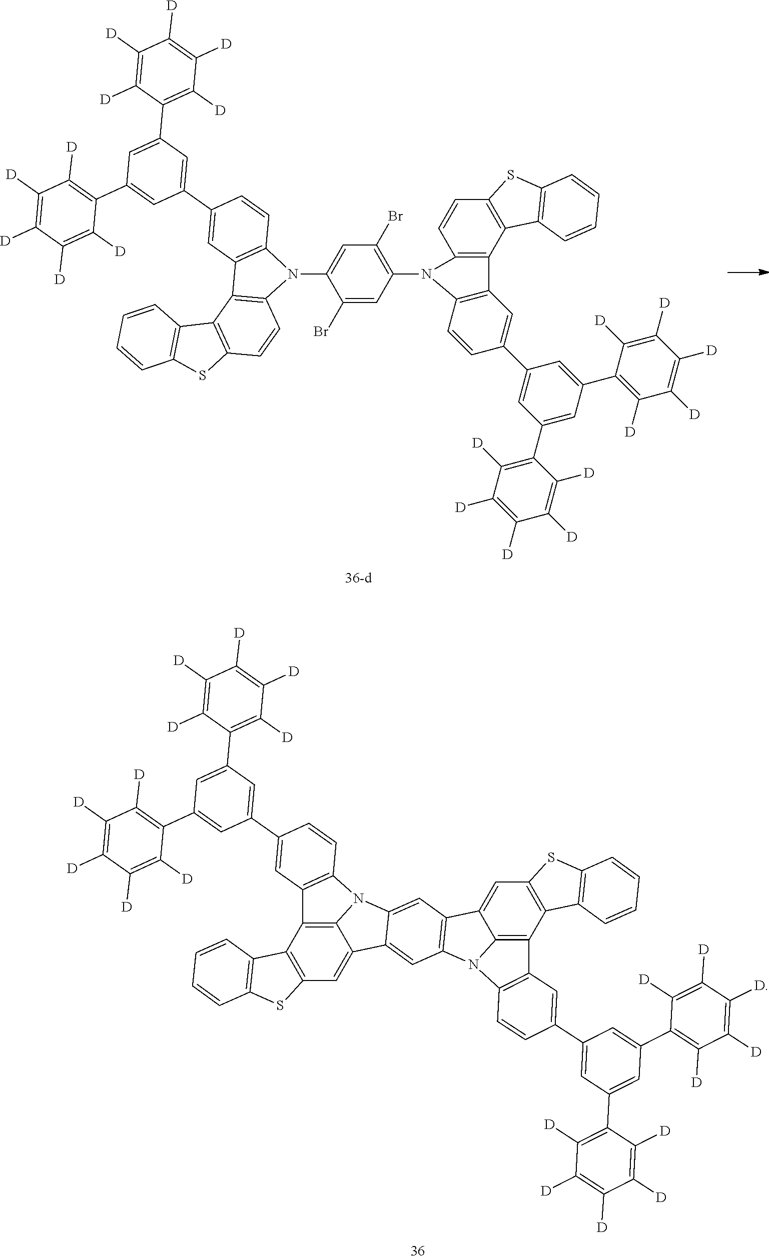

In an argon atmosphere, to a 250 mL-flask, Intermediate Compound 36-d (13.0 g, 10.3 mmol), palladium(II) acetate (0.46 g, 2.06 mmol), potassium carbonate (14.2 g, 103 mmol), and triphenylphosphine (2.7 g, 10.3 mmol) were added and dissolved in 100 mL of DMAC, and then the reaction solution was stirred at about 180° C. for about 12 hours. The reaction solution was cooled and then filtered to obtain a yellow solid, and then the yellow solid was washed with water and ethanol. The solid thus obtained was subjected to flash column utilizing CH₂Cl₂ as the eluent, and recrystallized with CH₂Cl₂ and hexane to obtain Compound 36 (yellow solid, 1.46 g, 1.3 mmol, 13%). The obtained compound was identified as Compound 36 through ESI-LCMS.

ESI-LCMS: [M]*: C₇₈H₂₄D₂₀N₂S₂. 1092.42

### (5) Synthesis of Compound 41

Fused Polycyclic Compound 41 according to an example may be synthesized, for example, by the reaction below:

### Synthesis of Intermediate Compound 41-a

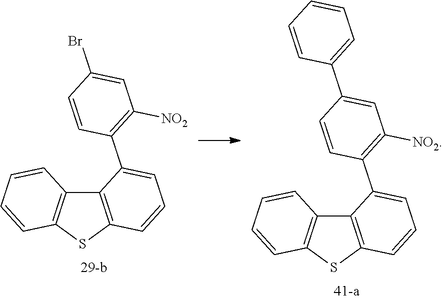 [SMILES: O=[N+]([O-])c1cc(Br)ccc1-c1cccc2sc3ccccc3c12 ; O=[N+]([O-])c1cc(-c2ccccc2)ccc1-c1cccc2sc3ccccc3c12]

In an argon atmosphere, to a 1 L-flask, Intermediate Compound 29-b (15.5 g, 40.3 mmol), phenylboronic acid (5.4 g, 44.3 mmol), Pd(PPh₃)₄ (2.33 g, 2.02 mmol), and potassium carbonate (16.7 g, 121 mmol) were added and dissolved in 250 mL of toluene, 50 mL of ethanol, and 100 mL of H₂O, and then the reaction solution was stirred at about 100° C. for about 12 hours. After being cooled, the reaction solution was extracted by adding water (300 mL) and ethyl acetate (200 mL) to collect organic layers, and the organic layers were dried over MgSO₄ and then filtered. In the filtrate, the solvent was removed under reduced pressure to obtain a solid. The solid thus obtained was purified and separated by silica gel column chromatography utilizing CH₂Cl₂ and hexane as the eluent to obtain Intermediate Compound 41-a (yellow solid, 12.0 g, 31.4 mmol, 78%). The obtained compound was identified as Intermediate Compound 41-a through ESI-LCMS.

ESI-LCMS: [M]⁺: C₂₄H₁₅NO₂S. 381.08

### Synthesis of Intermediate Compound 41-b

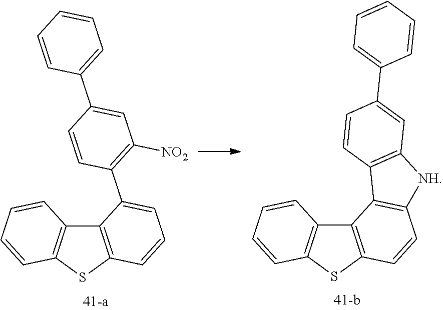 [SMILES: O=[N+]([O-])c1cc(-c2ccccc2)ccc1-c1cccc2sc3ccccc3c12 ; c1ccc(-c2ccc3c(c2)[nH]c2ccc4sc5ccccc5c4c23)cc1]

In an argon atmosphere, to a 500 mL-flask, Intermediate Compound 41-a (12.0 g, 31.4 mmol) and PPh₃ (18.1 g, 69.1 mmol) were added and dissolved in 125 mL of o-dichlorobenzene, and then the reaction solution was stirred at about 200° C. for about 12 hours. After the reaction solution was cooled, the solvent was removed under reduced pressure to obtain a solid. The solid thus obtained was filtered by silica gel pad utilizing CH₂Cl₂ and hexane as the eluent, and recrystallized with CH₂Cl₂ and hexane to obtain Intermediate Compound 41-b (white solid, 10.4 g, 29.8 mmol, 95%). The obtained compound was identified as Intermediate Compound 41-b through ESI-LCMS.

ESI-LCMS: [M]*: C₂₄H₁₅NS. 349.09.

### Synthesis of Intermediate Compound 41-c

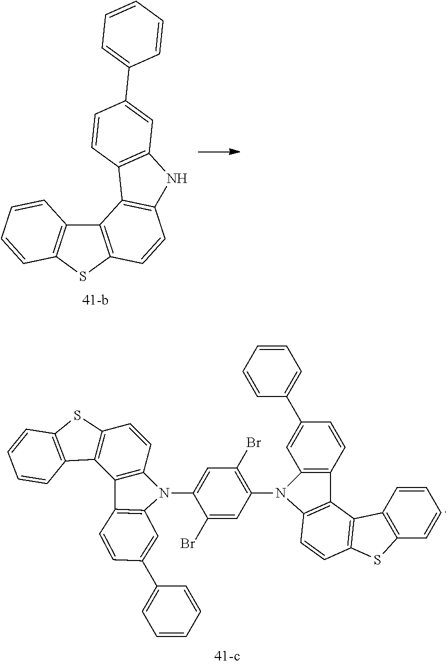 [SMILES: c1ccc(-c2ccc3c(c2)[nH]c2ccc4sc5ccccc5c4c23)cc1 ; Brc1cc(-n2c3cc(-c4ccccc4)ccc3c3c4c(ccc32)sc2ccccc24)c(Br)cc1-n1c2cc(-c3ccccc3)ccc2c2c3c(ccc21)sc1ccccc13]

In an argon atmosphere, to a 500 mL-flask, Intermediate Compound 41-b (10.4 g, 29.8 mmol), 1,4-dibromo-2,5-difluorobenzene (3.2 g, 11.9 mmol), and K₃PO₄ (7.6 g, 35.8 mmol) were dissolved in 200 mL of DMF, and then the reaction solution was stirred at about 150° C. for about 12 hours. After the reaction solution was cooled, the solvent was removed under reduced pressure to obtain a solid. The solid thus obtained was dissolved in CH₂Cl₂ and subjected to celite pad filter, and the filtrate was filtered with silica gel pad. Then, the solid was recrystallized by utilizing CH₂Cl₂ and methanol to obtain Intermediate Compound 41-c (white solid, 8.8 g, 9.4 mmol, 79%). The obtained compound was identified as Intermediate Compound 41-c through ESI-LCMS.

ESI-LCMS: [M]⁺: C₅₄H₃₀Br₂N₂S₂. 930.02

### Synthesis of Compound 41

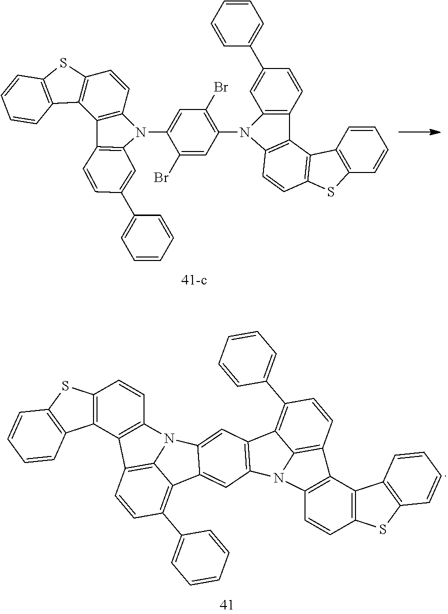 [SMILES: Brc1cc(-n2c3cc(-c4ccccc4)ccc3c3c4c(ccc32)sc2ccccc24)c(Br)cc1-n1c2cc(-c3ccccc3)ccc2c2c3c(ccc21)sc1ccccc13 ; c1ccc(-c2ccc3c4c5c(ccc4n4c6cc7c8c(-c9ccccc9)ccc9c%10c%11c(ccc%10n(c7cc6c2c34)c98)sc2ccccc2%11)sc2ccccc25)cc1]

In an argon atmosphere, to a 250 mL-flask, Intermediate Compound 41-c (8.8 g, 9.4 mmol), palladium(II) acetate (0.42 g, 1.9 mmol), potassium carbonate (13.0 g, 94 mmol), and triphenylphosphine (2.5 g, 9.4 mmol) were added and dissolved in 100 mL of DMAC, and then the reaction solution was stirred at about 180° C. for about 12 hours. The reaction solution was cooled and then filtered to obtain a yellow solid, and then the yellow solid was washed with water and ethanol. The solid thus obtained was subjected to flash column utilizing CH₂Cl₂ as the eluent, and recrystallized with CH₂Cl₂ and hexane to obtain Compound 41 (yellow solid, 1.4 g, 1.8 mmol, 19%). The obtained compound was identified as Compound 41 through ESI-LCMS.

ESI-LCMS: [M]*: C₅₄H₂₈N₂S₂. 768.17

### (6) Synthesis of Compound 71

Fused Polycyclic Compound 71 according to an example may be synthesized, for example, by the reaction below:

### Synthesis of Intermediate Compound 71-a

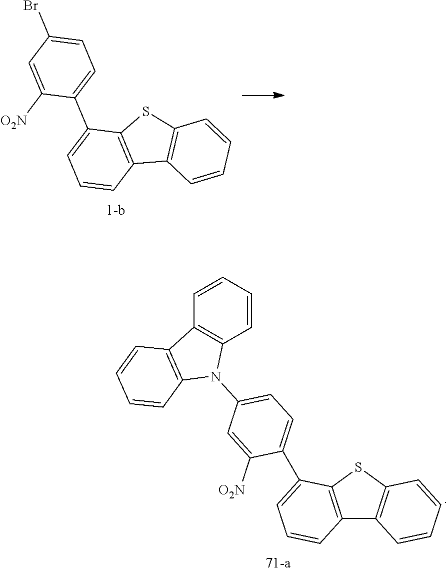 [SMILES: O=[N+]([O-])c1cc(Br)ccc1-c1cccc2c1sc1ccccc12 ; O=[N+]([O-])c1cc(-n2c3ccccc3c3ccccc32)ccc1-c1cccc2c1sc1ccccc12]

In an argon atmosphere, to a 2 L-flask, Intermediate Compound 1-b (20 g, 52 mmol), 9H-carbazole (9.6 g, 57.2 mmol), tris(dibenzylideneacetone)dipalladium(0) (2.4 g, 2.6 mmol), tris-tert-butyl phosphine solution 50% in toluene (2.4 mL, 5.2 mmol), and sodium tert-butoxide (10.0 g, 104 mmol) were dissolved in 500 mL of o-xylene, and then the reaction solution was stirred at about 150° C. for about 12 hours. After the reaction solution was cooled, the solvent was removed under reduced pressure, and the reaction solution was extracted by adding water (500 mL) and ethyl acetate (500 mL) to collect organic layers, and the organic layers were dried over MgSO₄ and then filtered. In the filtrate, the solvent was removed under reduced pressure to obtain a solid. The solid thus obtained was purified by silica gel chromatography utilizing CH₂Cl₂ and hexane as the eluent to obtain Intermediate Compound 71-a (white solid, 16.9 g, 35.9 mmol, 69%). The obtained compound was identified as Intermediate Compound 71-a through ESI-LCMS.

ESI-LCMS: [M]⁺: C₃₀H18N₂O₂S. 470.11

### Synthesis of Intermediate Compound 71-b

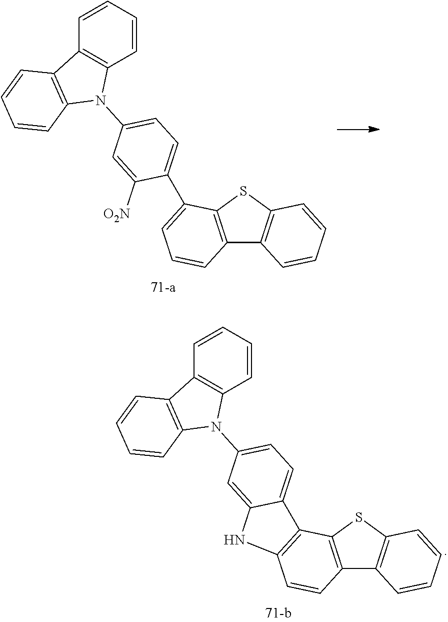 [SMILES: O=[N+]([O-])c1cc(-n2c3ccccc3c3ccccc32)ccc1-c1cccc2c1sc1ccccc12 ; c1ccc2c(c1)sc1c2ccc2[nH]c3cc(-n4c5ccccc5c5ccccc54)ccc3c21]

In an argon atmosphere, to a 500 mL-flask, Intermediate Compound 71-a (16.9 g, 35.9 mmol) and PPh₃ (20.7 g, 79 mmol) were added and dissolved in 125 mL of o-dichlorobenzene, and then the reaction solution was stirred at about 200° C. for about 12 hours. After the reaction solution was cooled, the solvent was removed under reduced pressure to obtain a solid. The solid thus obtained was filtered by silica gel pad utilizing CH₂Cl₂ and hexane as the eluent, and recrystallized with CH₂Cl₂ and hexane to obtain Intermediate Compound 71-b (white solid, 12.8 g, 29 mmol, 81%). The obtained compound was identified as Intermediate Compound 71-b through ESI-LCMS.

ESI-LCMS: [M]⁺: C₃₀H₁₈N₂S. 438.12.

### Synthesis of Intermediate Compound 71-c

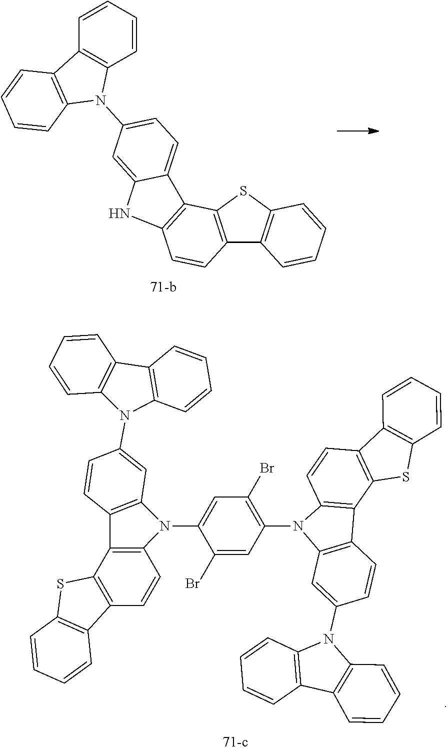 [SMILES: c1ccc2c(c1)sc1c2ccc2[nH]c3cc(-n4c5ccccc5c5ccccc54)ccc3c21 ; Brc1cc(-n2c3cc(-n4c5ccccc5c5ccccc54)ccc3c3c4sc5ccccc5c4ccc32)c(Br)cc1-n1c2cc(-n3c4ccccc4c4ccccc43)ccc2c2c3sc4ccccc4c3ccc21]

In an argon atmosphere, to a 500 mL-flask, Intermediate Compound 71-b (12.8 g, 29 mmol), 1,4-dibromo-2,5-difluorobenzene (3.2 g, 11.6 mmol), and K₃PO₄ (7.4 g, 34.8 mmol) were dissolved in 200 mL of DMF, and then the reaction solution was stirred at about 150° C. for about 12 hours. After the reaction solution was cooled, the solvent was removed under reduced pressure to obtain a solid. The solid thus obtained was dissolved in CH₂Cl₂ and subjected to celite pad filter, and the filtrate was filtered with silica gel pad. Then, the solid was recrystallized by utilizing CH₂Cl₂ and methanol to obtain Intermediate Compound 71-c (white solid, 8.4 g, 7.5 mmol, 65%). The obtained compound was identified as Intermediate Compound 71-c through ESI-LCMS.

ESI-LCMS: [M]⁺: C₆₆H₃₆Br₂N₄S₂. 1108.07

### Synthesis of Compound 71

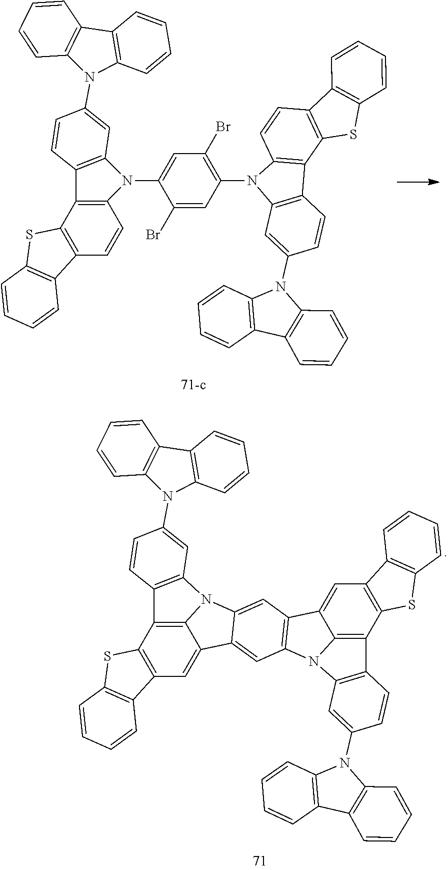 [SMILES: Brc1cc(-n2c3cc(-n4c5ccccc5c5ccccc54)ccc3c3c4sc5ccccc5c4ccc32)c(Br)cc1-n1c2cc(-n3c4ccccc4c4ccccc43)ccc2c2c3sc4ccccc4c3ccc21 ; c1ccc2c(c1)sc1c2cc2c3cc4c(cc3n3c5cc(-n6c7ccccc7c7ccccc76)ccc5c1c23)c1cc2c3ccccc3sc2c2c3ccc(-n5c6ccccc6c6ccccc65)cc3n4c12]

In an argon atmosphere, to a 250 mL-flask, Intermediate Compound 71-c (8.4 g, 7.5 mmol), palladium(II) acetate (0.34 g, 1.5 mmol), potassium carbonate (10.4 g, 75 mmol), and triphenylphosphine (2.0 g, 7.5 mmol) were added and dissolved in 75 mL of DMAC, and then the reaction solution was stirred at about 180° C. for about 12 hours. The reaction solution was cooled and then filtered to obtain a yellow solid, and then the yellow solid was washed with water and ethanol. The solid thus obtained was subjected to flash column utilizing CH₂Cl₂ as the eluent, and recrystallized with CH₂Cl₂ and hexane to obtain Compound 71 (yellow solid, 0.85 g, 0.9 mmol, 12%). The obtained compound was identified as Compound 71 through ESI-LCMS.

ESI-LCMS: [M]*: C₆₆H₃₄N₄S₂. 946.22

### (7) Synthesis of Compound 79

Fused Polycyclic Compound 79 according to an example may be synthesized by, for example, the reaction below:

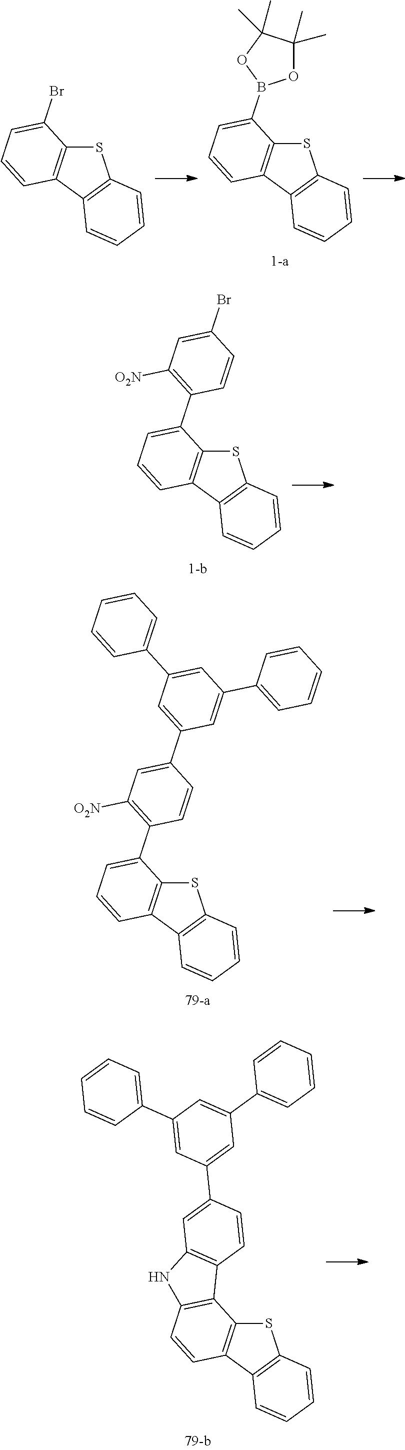 [SMILES: Brc1cccc2c1sc1ccccc12 ; CC1(C)OB(c2cccc3c2sc2ccccc23)OC1(C)C ; O=[N+]([O-])c1cc(Br)ccc1-c1cccc2c1sc1ccccc12 ; O=[N+]([O-])c1cc(-c2cc(-c3ccccc3)cc(-c3ccccc3)c2)ccc1-c1cccc2c1sc1ccccc12 ; c1ccc(-c2cc(-c3ccccc3)cc(-c3ccc4c(c3)[nH]c3ccc5c6ccccc6sc5c34)c2)cc1]

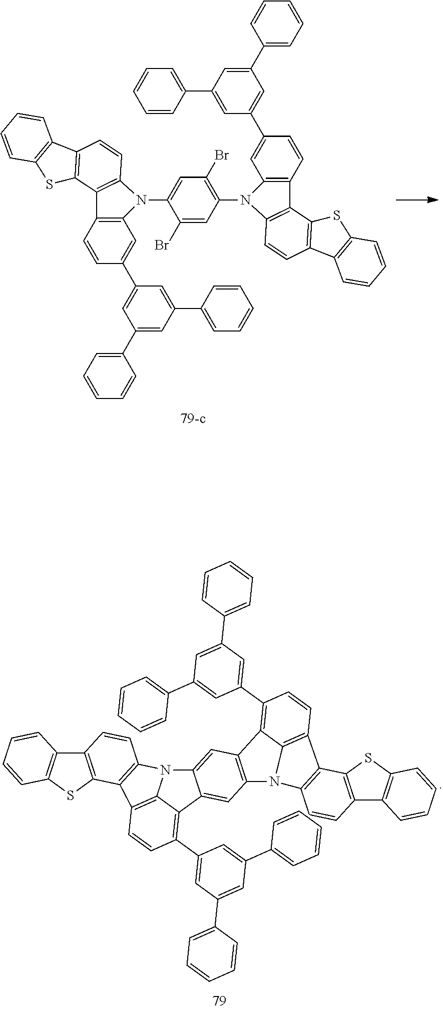 [SMILES: Brc1cc(-n2c3cc(-c4cc(-c5ccccc5)cc(-c5ccccc5)c4)ccc3c3c4sc5ccccc5c4ccc32)c(Br)cc1-n1c2cc(-c3cc(-c4ccccc4)cc(-c4ccccc4)c3)ccc2c2c3sc4ccccc4c3ccc21 ; c1ccc(-c2cc(-c3ccccc3)cc(-c3ccc4c5c6sc7ccccc7c6ccc5n5c6cc7c8c(-c9cc(-c%10ccccc%10)cc(-c%10ccccc%10)c9)ccc9c%10c%11sc%12ccccc%12c%11ccc%10n(c7cc6c3c45)c98)c2)cc1]

Compound 79 (yield: 10%) was obtained in substantially the same manner as in Synthesis of Compound 41 except for utilizing Intermediate Compound 1-b instead of Intermediate Compound 29-b in Synthesis of Compound 41 and utilizing 1,1′:3′,1″-terphenyl-5′-boronic acid instead of phenylboronic acid. The obtained compound was identified as Compound 79 through ESI-LCMS.

ESI-LCMS: [M]*: C₇₈H₄₄N₂S₂. 1072.29

### (8) Synthesis of Compound 121

Fused Polycyclic Compound 121 according to an example may be synthesized, for example, by the reaction below:

### Synthesis of Intermediate Compound 121-a

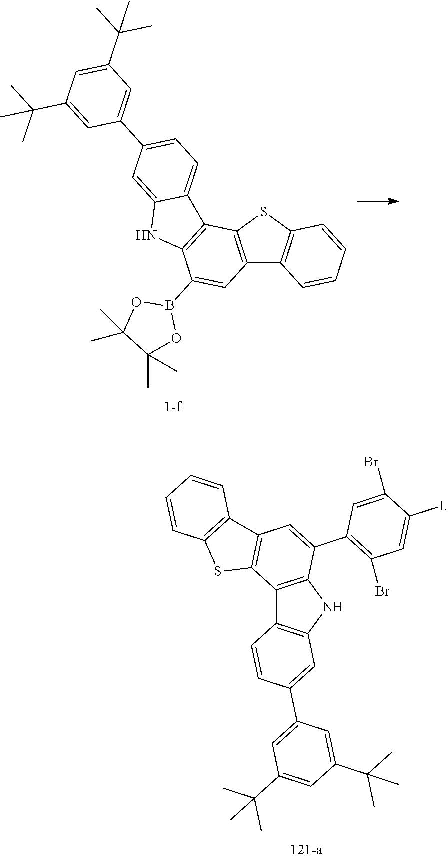 [SMILES: CC(C)(C)c1cc(-c2ccc3c(c2)[nH]c2c(B4OC(C)(C)C(C)(C)O4)cc4c5ccccc5sc4c23)cc(C(C)(C)C)c1 ; CC(C)(C)c1cc(-c2ccc3c(c2)[nH]c2c(-c4cc(Br)c(I)cc4Br)cc4c5ccccc5sc4c23)cc(C(C)(C)C)c1]

In an argon atmosphere, to a 500 mL-flask, Intermediate Compound 1-f (8 g, 14 mmol), 1,4-dibromo-2,5-diiodobenzene (13.7 g, 28 mmol), Pd(PPh₃)₄ (0.49 g, 0.42 mmol), and sodium carbonate (3.0 g, 28 mmol) were added and dissolved in 140 mL of toluene, 28 mL of DMSO, and 8.4 mL of H₂O, and then the reaction solution was stirred at about 90° C. for about 24 hours. After the reaction solution was cooled, toluene was removed under reduced pressure, and then water (100 mL) was added thereto to filter out a solid. Then, the solid was purified by silica gel column chromatography to obtain Intermediate Compound 121-a (white solid, 7.5 g, 9.1 mmol, 65%). The obtained compound was identified as Intermediate Compound 121-a through ESI-LCMS.

ESI-LCMS: [M]⁺: C₃₈H₃₂Br₂1NS. 820.96

### Synthesis of Intermediate Compound 121-b

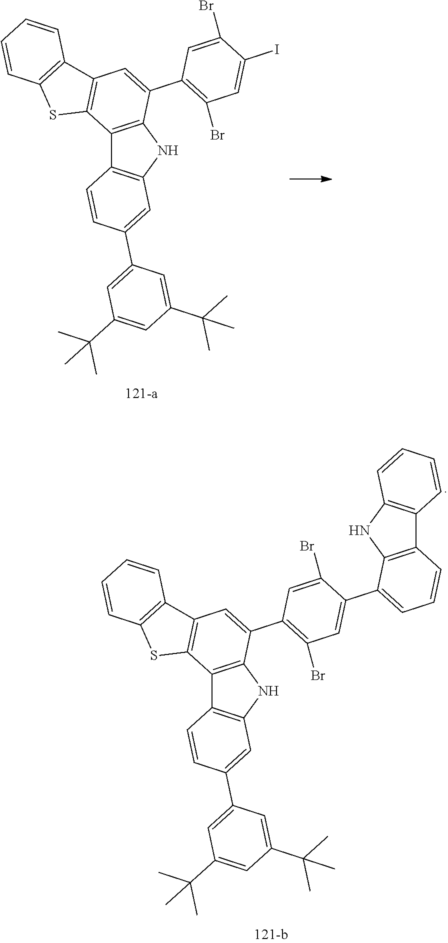 [SMILES: CC(C)(C)c1cc(-c2ccc3c(c2)[nH]c2c(-c4cc(Br)c(I)cc4Br)cc4c5ccccc5sc4c23)cc(C(C)(C)C)c1 ; CC(C)(C)c1cc(-c2ccc3c(c2)[nH]c2c(-c4cc(Br)c(-c5cccc6c5[nH]c5ccccc56)cc4Br)cc4c5ccccc5sc4c23)cc(C(C)(C)C)c1]

In an argon atmosphere, to a 250 mL-flask, Intermediate Compound 121-a (7.5 g, 9.1 mmol), 1-(4,4,5,5-tetramethyl-1,3,2-dioxaborolan-2-yl)-9H-carbazole (5.3 g, 18.2 mmol), Pd(PPh₃)₄ (0.53 g, 0.46 mmol), and sodium carbonate (4.8 g, 45.5 mmol) were added and dissolved in 100 mL of toluene, 20 mL of DMSO, and 6 mL of H₂O, and then the reaction solution was stirred at about 90° C. for about 24 hours. After the reaction solution was cooled, toluene was removed under reduced pressure, and then water (100 mL) was added thereto to filter out a solid. Then, the solid was purified by silica gel column chromatography to obtain Intermediate Compound 121-b (white solid, 5.7 g, 6.6 mmol, 72%). The obtained compound was identified as Intermediate Compound 121-b through ESI-LCMS.

### Synthesis of Compound 121

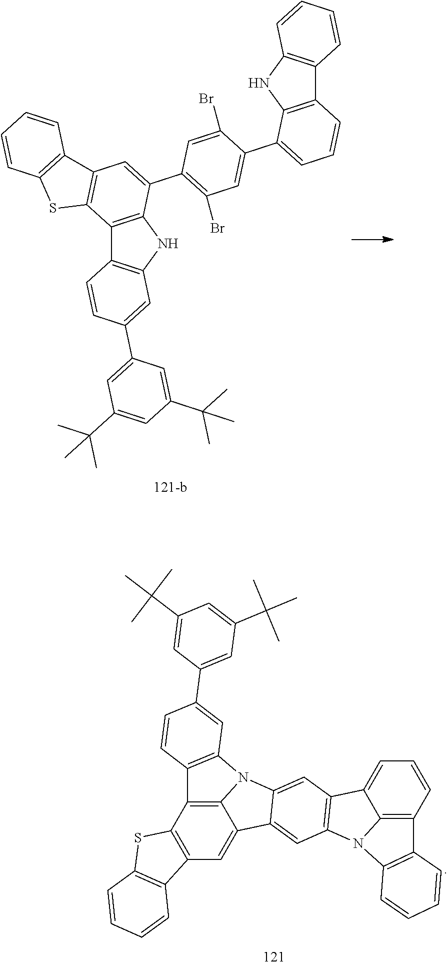 [SMILES: CC(C)(C)c1cc(-c2ccc3c(c2)[nH]c2c(-c4cc(Br)c(-c5cccc6c5[nH]c5ccccc56)cc4Br)cc4c5ccccc5sc4c23)cc(C(C)(C)C)c1 ; CC(C)(C)c1cc(-c2ccc3c4c5sc6ccccc6c5cc5c6cc7c(cc6n(c3c2)c54)c2cccc3c4ccccc4n7c32)cc(C(C)(C)C)c1]

In an argon atmosphere, to a 500 mL-flask, Intermediate Compound 121-b (5.7 g, 6.6 mmol), CuI (0.63 g, 3.3 mmol), potassium acetate (2.7 g, 19.8 mmol), and L-proline (0.38 g, 3.3 mmol) were added and dissolved in 125 mL of DMSO, and then the reaction solution was stirred at about 150° C. for about 6 hours. The reaction solution was cooled and then filtered to obtain a yellow solid, and then the yellow solid was washed with water and ethanol. The solid thus obtained was filtered by silica gel pad utilizing CH₂Cl₂ as the eluent, and recrystallized with CH₂Cl₂ and hexane to obtain Compound 121 (yellow solid, 2.4 g, 3.4 mmol, 51%). The obtained compound was identified as Compound 121 through ESI-LCMS.

ESI-LCMS: [M]⁺: C₅₀H₃₈N₂S. 698.28

### 2. Manufacture and Evaluation of Light Emitting Device Including Fused Polycyclic Compound

The light emitting device of an example including the fused polycyclic compound of an example in an emission layer was manufactured as follows. Fused polycyclic compounds of Compounds 1, 12, 29, 36, 41, 71, 79, and 121, which are Example Compounds as described above were utilized as dopant materials for the emission layers to manufacture the light emitting devices of Examples 1 to 8, respectively. Comparative Examples 1 to 5 correspond to the light emitting devices manufactured by utilizing Comparative Example Compounds C₁ to C₅ as dopant materials for the emission layers, respectively.

### Example Compounds

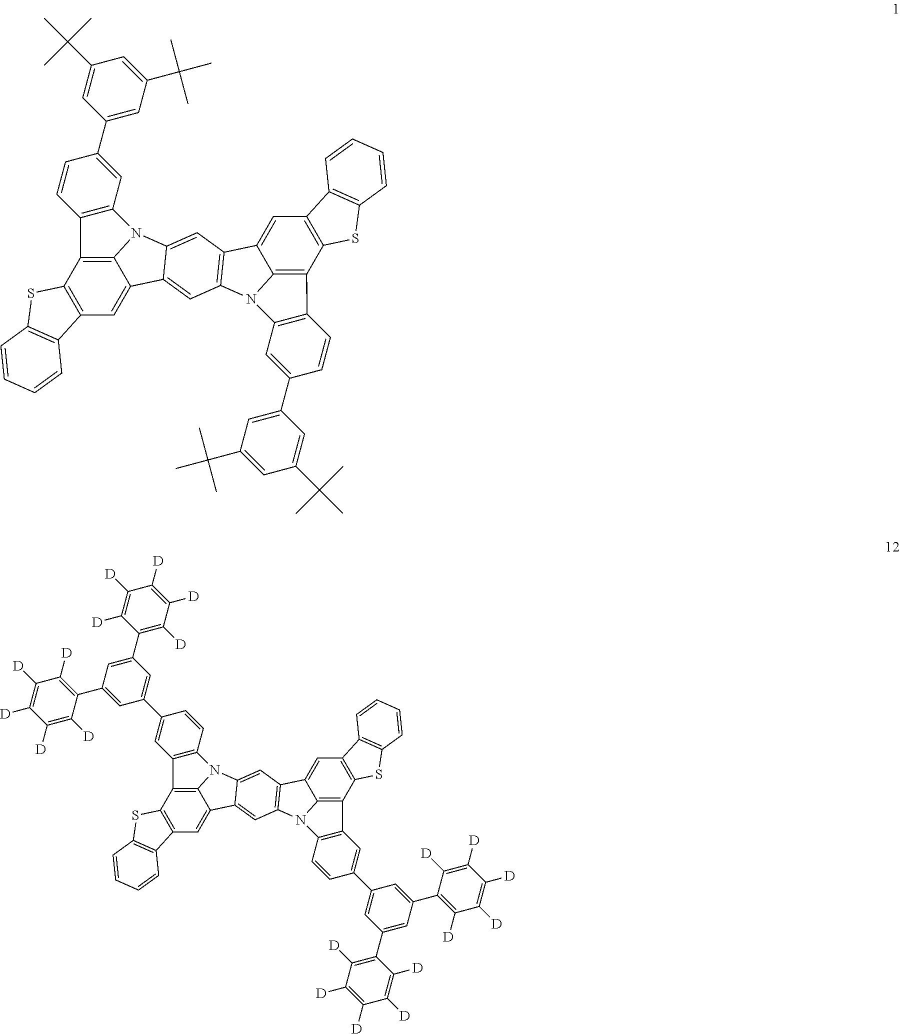 [SMILES: CC(C)(C)c1cc(-c2ccc3c4c5sc6ccccc6c5cc5c6cc7c(cc6n(c3c2)c54)c2cc3c4ccccc4sc3c3c4ccc(-c5cc(C(C)(C)C)cc(C(C)(C)C)c5)cc4n7c23)cc(C(C)(C)C)c1 ; [2H]c1c([2H])c([2H])c(-c2cc(-c3ccc4c(c3)c3c5sc6ccccc6c5cc5c6cc7c(cc6n4c53)c3cc4c5ccccc5sc4c4c5cc(-c6cc(-c8c([2H])c([2H])c([2H])c([2H])c8[2H])cc(-c8c([2H])c([2H])c([2H])c([2H])c8[2H])c6)ccc5n7c34)cc(-c3c([2H])c([2H])c([2H])c([2H])c3[2H])c2)c([2H])c1[2H]]

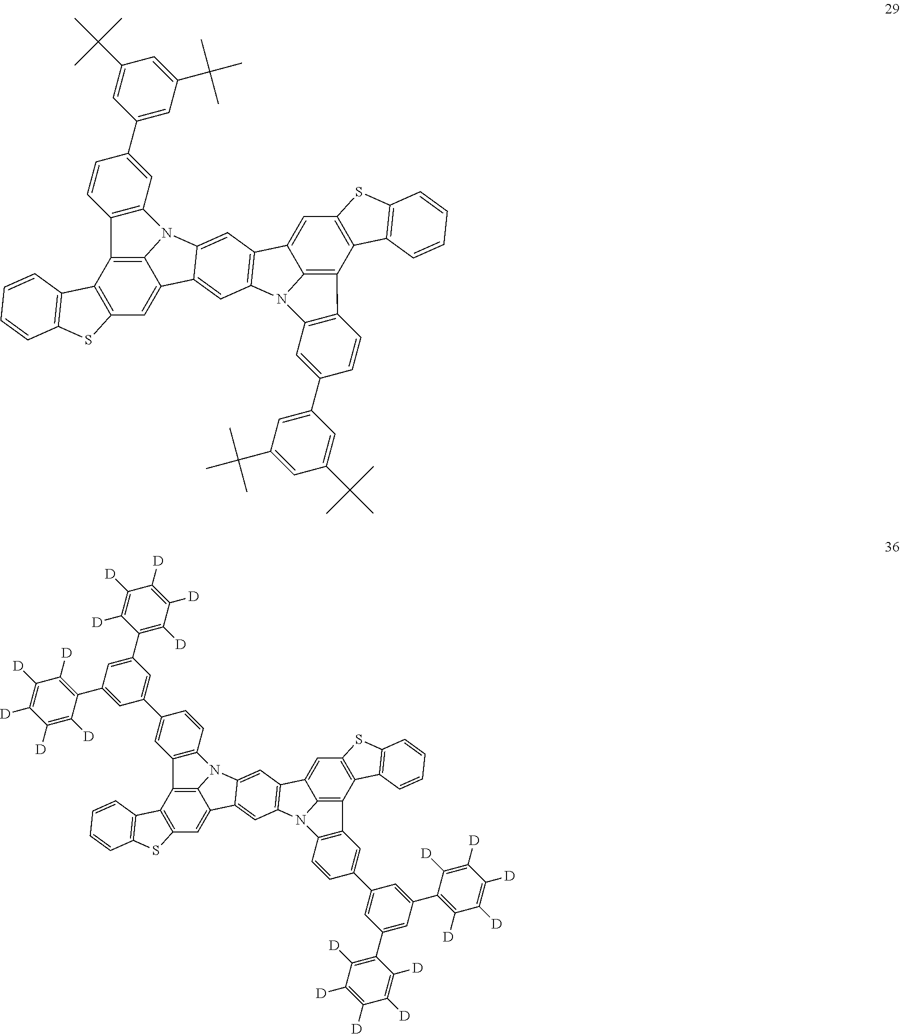 [SMILES: CC(C)(C)c1cc(-c2ccc3c4c5c(cc6c7cc8c(cc7n(c3c2)c64)c2cc3sc4ccccc4c3c3c4ccc(-c6cc(C(C)(C)C)cc(C(C)(C)C)c6)cc4n8c23)sc2ccccc25)cc(C(C)(C)C)c1 ; [2H]c1c([2H])c([2H])c(-c2cc(-c3ccc4c(c3)c3c5c(cc6c7cc8c(cc7n4c63)c3cc4sc6ccccc6c4c4c6cc(-c7cc(-c9c([2H])c([2H])c([2H])c([2H])c9[2H])cc(-c9c([2H])c([2H])c([2H])c([2H])c9[2H])c7)ccc6n8c34)sc3ccccc35)cc(-c3c([2H])c([2H])c([2H])c([2H])c3[2H])c2)c([2H])c1[2H]]

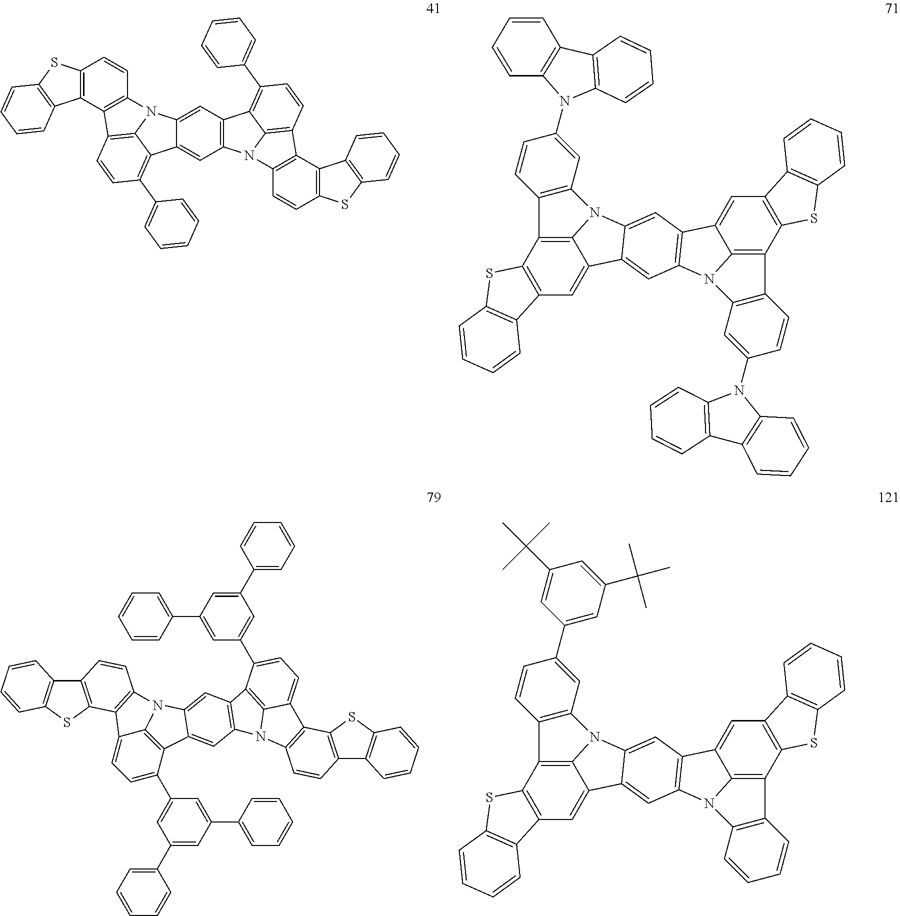

### Comparative Example Compounds

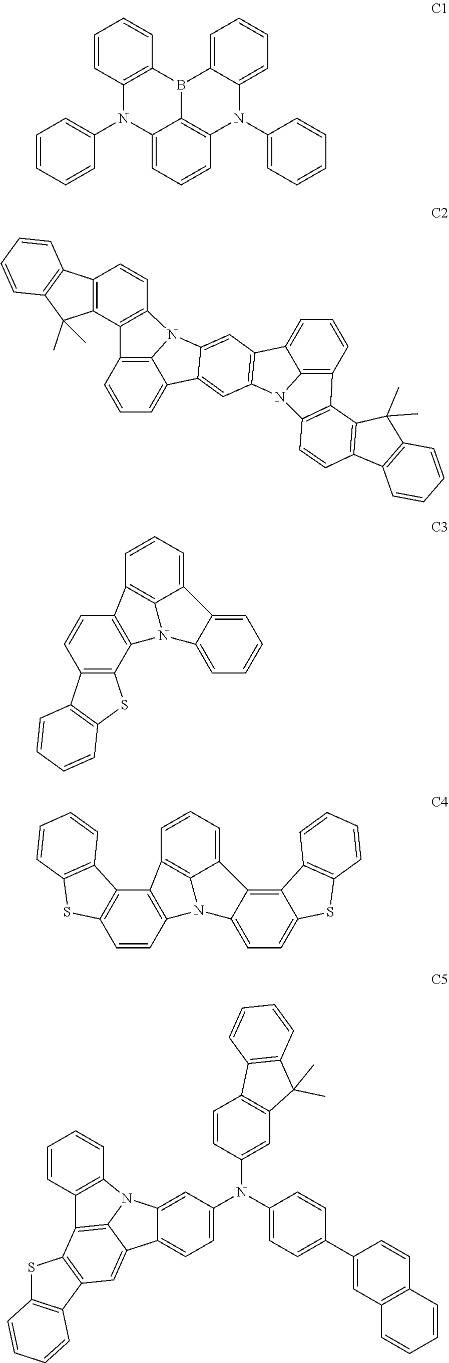

### Manufacture of Light Emitting Device

With respect to the light emitting devices of Examples and Comparative Examples, an ITO glass substrate was cut to a size of about 50 mm×50 mm×0.7 mm, washed by ultrasonic waves utilizing isopropyl alcohol and distilled water for about 5 minutes, respectively, and then irradiated with ultraviolet rays for about 30 minutes and cleansed by exposing to ozone, and then installed on a vacuum deposition apparatus. Then, NPD was utilized to form a hole injection layer having a thickness of about 300 Å, HT-1-19 was utilized to form a hole transport layer having a thickness of about 200 Å, and then CzSi was utilized to form an emission auxiliary layer having a thickness of about 100 Å. Then, a host compound in which the second compound and the third compound according to an embodiment were mixed in an amount (e.g., weight ratio) of about 1:1, the fourth compound, and Example Compound or Comparative Example Compound were co-deposited in a weight ratio of about 82:15:3 to form a 200 Å-thick emission layer, and TSPO1 was utilized to form a 200 Å-thick hole blocking layer. Next, TPBI, an electron transporting compound, was utilized to form a 300 Å-thick electron transport layer, and LiF was utilized to form a 10 Å-thick electron injection layer. A₁ was then utilized to form a 3,000 Å-thick second electrode to form a LiF/Al electrode. Each layer was formed by a vacuum deposition method. Meanwhile, HT33 from among the compounds in Compound Group 2 as described above was utilized as the second compound, and ETH66 from among the compounds in Compound Group 3 as described above was utilized as the third compound, and AD-37 from among the compounds in Compound Group 4 as described above was utilized as the fourth compound.

Compounds utilized for manufacturing the light emitting devices of Examples and Comparative Examples are disclosed below. The materials below were utilized to manufacture the elements by subjecting commercial products to sublimation purification.

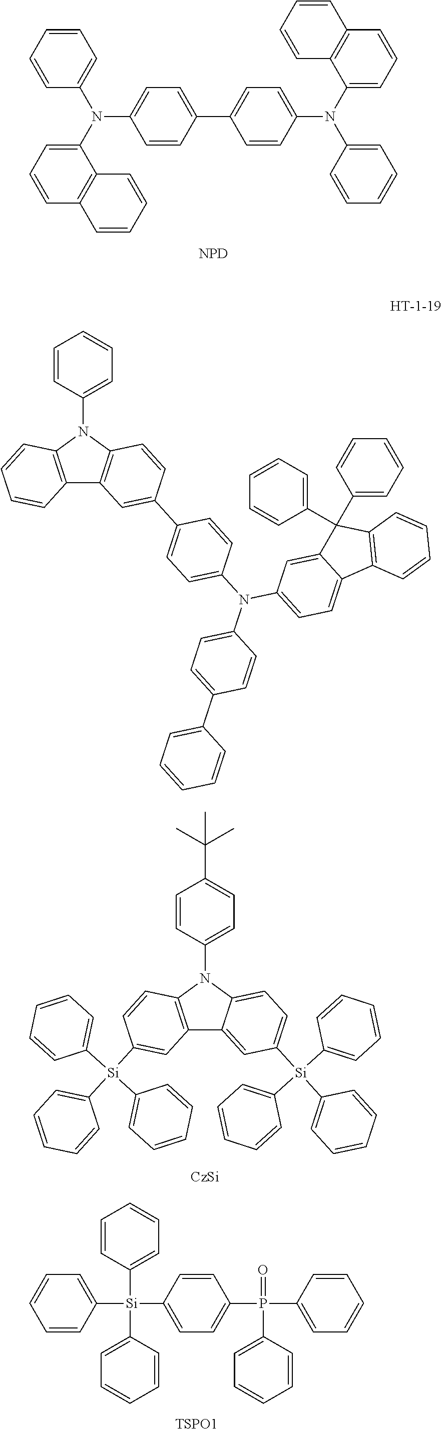

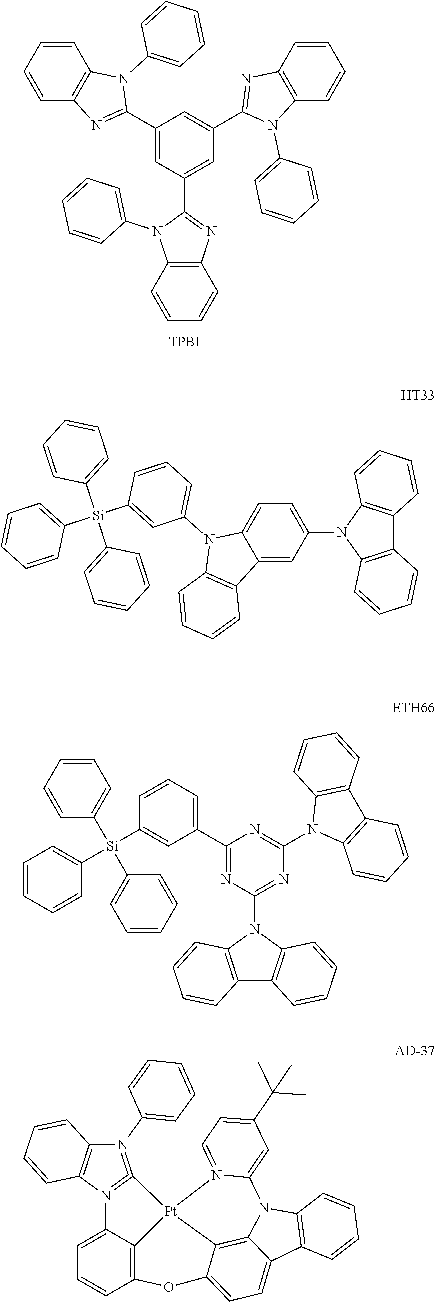

### Evaluation of Light Emitting Device Characteristics

Device efficiency and device service life of the light emitting devices manufactured with Example Compounds 1, 12, 29, 36, 41, 71, 79, and 121, and Comparative Example Compounds C₁ to C₅ as described above were evaluated. Evaluation results of the light emitting devices of Examples 1 to 8, and Comparative Examples 1 to 5 are listed in Table 1. In the characteristic evaluation results of Examples and Comparative Examples listed in Table 1, driving voltages and current densities were measured by utilizing V7000 OLED IVL Test System (Polaronix). To evaluate the characteristics of the light emitting devices manufactured in Examples 1 to 8 and Comparative Examples 1 to 5, driving voltages and efficiencies (cd/A) at a current density of 10 mA/cm² were measured, and the relative device service life was first measured as the time duration it took for the brightness of each device to deteriorate from an initial value to 50% of the initial brightness value when the device was continuously operated at a current density of 10 mA/cm², and then the time duration obtained for each device was compared to that of Comparative Example 1, and then the evaluation result was presented as a ratio between the two.

TABLE 1

| | Second | | | | | | |
|---|---|---|---|---|---|---|---|
| | compound/ | | | | | | Service |
| | Third | | | Driving | | Luminescence | life |
| | compound | Fourth | First | voltage | Efficiency | wavelength | ratio |
| | (5:5) | compound | compound | (V) | (cd/A) | (nm) | (T₉₅) |
| Example 1 | HT33/ETH66 | AD-37 | Example | 4.5 | 22.3 | 455 | 3.1 |
| | | | Compound 1 | | | | |
| Example 2 | HT33/ETH66 | AD-37 | Example | 4.4 | 24.3 | 456 | 3.6 |
| | | | Compound 12 | | | | |
| Example 3 | HT33/ETH66 | AD-37 | Example | 4.3 | 25.1 | 460 | 5.3 |
| | | | Compound 29 | | | | |
| Example 4 | HT33/ETH66 | AD-37 | Example | 4.3 | 23.9 | 460 | 5.5 |
| | | | Compound 36 | | | | |
| Example 5 | HT33/ETH66 | AD-37 | Example | 4.5 | 25.2 | 460 | 6.8 |
| | | | Compound 41 | | | | |
| Example 6 | HT33/ETH66 | AD-37 | Example | 4.3 | 24.0 | 457 | 5.0 |
| | | | Compound 71 | | | | |
| Example 7 | HT33/ETH66 | AD-37 | Example | 4.4 | 24.8 | 457 | 4.7 |
| | | | Compound 79 | | | | |
| Example 8 | HT33/ETH66 | AD-37 | Example | 4.3 | 21.9 | 455 | 3.0 |
| | | | Compound 121 | | | | |
| Comparative | HT33/ETH66 | AD-37 | Comparative | 4.8 | 19.2 | 455 | 1 |
| Example 1 | | | Example | | | | |
| | | | Compound C1 | | | | |
| Comparative | HT33/ETH66 | AD-37 | Comparative | 4.6 | 16.2 | 460 | 2.3 |
| Example 2 | | | Example | | | | |
| | | | Compound C2 | | | | |
| Comparative | HT33/ETH66 | AD-37 | Comparative | 4.9 | 17.5 | 450 | 1.6 |
| Example 3 | | | Example | | | | |
| | | | Compound C3 | | | | |
| Comparative | HT33/ETH66 | AD-37 | Comparative | 4.8 | 18.1 | 454 | 1.8 |
| Example 4 | | | Example | | | | |
| | | | Compound C4 | | | | |
| Comparative | HT33/ETH66 | AD-37 | Comparative | 4.7 | 17.8 | 455 | 2.0 |
| Example 5 | | | Example | | | | |
| | | | Compound C5 | | | | |

Referring to the results of Table 1, it may be confirmed that Examples of the light emitting devices in which the fused polycyclic compounds according to examples of the present disclosure are each utilized as a luminescent material exhibit lower driving voltages, and have improved luminous efficiency and service life characteristics as compared with Comparative Examples.

Example Compounds each include a fused ring core in which a carbazole group is fused to an indolocarbazole moiety and has a structure in which at least one benzene ring is fused via a sulfur atom to the fused ring core, and thus high luminous efficiency and long service life may be achieved.

The Example Compounds may each have a structure in which a first carbazole group is fused to a first benzene ring from among first to third benzene rings constituting the indolocarbazole moiety. In this case, the nitrogen atom of the indolocarbazole group and the nitrogen atom of the first carbazole group may be linked at the para-position with respect to the first benzene ring. Accordingly, Example Compounds may have improved delayed fluorescence characteristics by the multiple resonance effects, and may exhibit improved service life characteristics due to high material stability. In addition, Example Compounds each include a structure in which at least one substituent represented by Formula 2 containing a sulfur atom is fused at the fused ring core, thereby triplet excitons may be harvested rapidly from singlet excitons due to the amplified reverse intersystem crossing, and thus when Example Compounds are utilized as a delayed fluorescence emitting material, the luminous efficiency of the light emitting device may be improved. The organic electroluminescence device of an example includes the fused polycyclic compound of an example as a dopant of a thermally activated delayed fluorescence (TADF) light emitting device, and thus may achieve high device efficiency and long service life in a blue wavelength region, particularly, a deep blue wavelength region.

It may be confirmed that Comparative Example Compound C₁ included in Comparative Example 1 contains a boron atom as an atom constituting the fused ring, and thus when applied to the device, the driving voltage is higher and the luminous efficiency and device service life are reduced as compared with Examples.

When Example 7 and Comparative Example 2 including Example Compound 79 and Comparative Example Compound C2, respectively, having a similar structure are compared, it may be confirmed that Example 7 including Example Compound 79 in which a sulfur atom is introduced into a fused ring core in which a carbazole group is fused at an indolocarbazole moiety has significantly improved luminous efficiency and service life as compared with Comparative Example 2. On the other hand, it may be confirmed that Comparative Example Compound C2 included in Comparative Example 2 includes a fused ring core in which a carbazole is fused at an indolocarbazole moiety, but a sulfur atom is not introduced into the fused ring core, and thus when applied to the device, the luminous efficiency and device service life are reduced. In particular, when Comparative Example 1 and Comparative Example 2 are compared, the compound having a fused ring structure in which the carbazole group is fused at the indolocarbazole moiety like Comparative Example Compound C2 exhibits a relatively higher difference (ΔEst) between the lowest triplet exciton energy level (T1 level) and the lowest singlet exciton energy level (S₁ level) than Comparative Example Compound C1 which is a boron-based polycyclic compound, and thus when applied to the light emitting device, the luminous efficiency may be reduced. On the other hand, in the case of Example Compounds, at least one substituent represented by Formula 2 containing a sulfur atom is fused at the fused ring core, and thus may be expected to have an improvement in the luminous efficiency due to the amplified speed of the reverse intersystem crossing even though the difference between the lowest triplet exciton energy level and the lowest singlet exciton energy level is somewhat large.

It may be confirmed that Comparative Example Compounds C3 to C5 included in Comparative Examples 3 to 5 include a structure in which a benzene ring is fused at the indolocarbazole moiety with a sulfur atom located therebetween, but the carbazole is not fused at the indolocarbazole moiety, and thus, Comparative Example Compounds C3 to C5 have higher driving voltages than Examples, and when applied to the device, the luminous efficiencies and service lives are reduced.

The light emitting device of an embodiment may exhibit improved device characteristics with relatively higher efficiency and a longer service life as compared with the related art devices.

The fused polycyclic compound of an embodiment may be included in the emission layer of the light emitting device to contribute to higher efficiency and a longer service life of the light emitting device.

As used herein, the term “and/or” includes any and all combinations of one or more of the associated listed items. Throughout the disclosure, the expression “at least one of a, b or c”, “at least one selected from a, b, and c”, “at least one selected from the group consisting of a, b, and c”, “at least one from among a, b, and c”, etc., indicates only a, only b, only c, both (e.g., simultaneously) a and b, both (e.g., simultaneously) a and c, both (e.g., simultaneously) b and c, all of a, b, and c, or variations thereof.

The use of “may” when describing embodiments of the present disclosure refers to “one or more embodiments of the present disclosure”.

As used herein, the terms “substantially”, “about”, and similar terms are used as terms of approximation and not as terms of degree, and are intended to account for the inherent deviations in measured or calculated values that would be recognized by those of ordinary skill in the art. “About” or “approximately,” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations, or within ±30%, 20%, 10%, 5% of the stated value.

Any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

The display device, and/or any other relevant devices or components according to embodiments of the present invention described herein may be implemented utilizing any suitable hardware, firmware (e.g. an application-specific integrated circuit), software, or a combination of software, firmware, and hardware. For example, the various components of the device may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments of the present disclosure.

Although the present disclosure has been described with reference to a preferred embodiment of the present disclosure, it will be understood that the present disclosure should not be limited to these preferred embodiments but one or more suitable changes and modifications can be made by those skilled in the art without departing from the spirit and scope of the present disclosure.

Accordingly, the technical scope of the present disclosure is not intended to be limited to the contents set forth in the detailed description of the specification, but is intended to be defined by the appended claims, and equivalents thereof.

What is claimed is:

1. A light emitting device comprising:
a first electrode;
a second electrode facing the first electrode; and
an emission layer between the first electrode and the second electrode,
wherein the emission layer comprises a first compound represented by Formula 1: 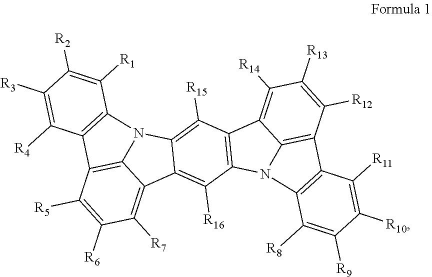
wherein, in Formula 1,
R₁ to R₁₆ are each independently a hydrogen atom, a deuterium atom, a halogen atom, a cyano group, a substituted or unsubstituted silyl group, a substituted or unsubstituted amine group, a substituted or unsubstituted oxy group, a substituted or unsubstituted thio group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and/or bonded to an adjacent group to form a ring,
at least one pair of adjacent groups selected from among R₁ to R₁₄ are bonded to each other to form a substituent represented by Formula 2: 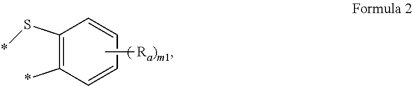
wherein, in Formula 2,
—* is a bonding site to a core of Formula 1,
Rₐ is a hydrogen atom, a deuterium atom, a halogen atom, a cyano group, a substituted or unsubstituted silyl group, a substituted or unsubstituted amine group, a substituted or unsubstituted oxy group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and/or bonded to an adjacent group to form a ring, and
m1 is an integer of 0 to 4.

2. The light emitting device of claim 1, wherein the first compound represented by Formula 1 is represented by Formula 3-1 or Formula 3-2: 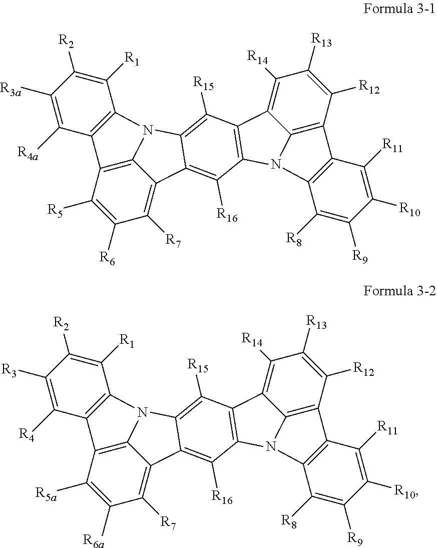
wherein, in Formula 3-1,
R₃ₐ and R₄ₐ are bonded to each other to form the substituent represented by Formula 2, and
wherein, in Formula 3-2,
R₅ₐ and R₆ₐ are bonded to each other to form the substituent represented by Formula 2, and
R₁ to R₁₆ are the same as defined in Formula 1.

3. The light emitting device of claim 1, wherein the first compound represented by Formula 1 is represented by Formula 4-1 or Formula 4-2: 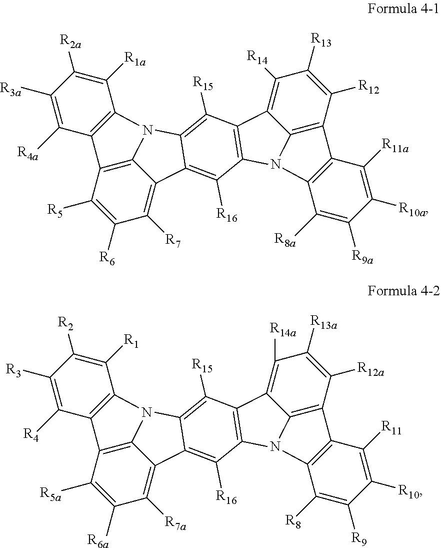
wherein, in Formula 4-1 and Formula 4-2,
R₁ₐ to R₁₄ₐ are each independently a hydrogen atom, a deuterium atom, a halogen atom, a cyano group, a substituted or unsubstituted silyl group, a substituted or unsubstituted amine group, a substituted or unsubstituted oxy group, a substituted or unsubstituted thio group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and/or bonded to an adjacent group to form a ring,
wherein, in Formula 4-1,
one pair of adjacent groups selected from among R₁ₐ to R₄ₐ are bonded to each other to form the substituent represented by Formula 2, and one pair of adjacent groups selected from among R₈ₐ to R₁₁ₐ are bonded to each other to form the substituent represented by Formula 2, and
wherein, in Formula 4-2,
one pair of adjacent groups selected from among R₅ₐ to R₇ₐ are bonded to each other to form the substituent represented by Formula 2, and one pair of adjacent groups selected from among R₁₂ₐ to R₁₄ₐ are bonded to each other to form the substituent represented by Formula 2, and
R₁ to R₁₆ are the same as defined in Formula 1.

4. The light emitting device of claim 1, wherein the first compound represented by Formula 1 is represented by Formula 5-1 or Formula 5-2: 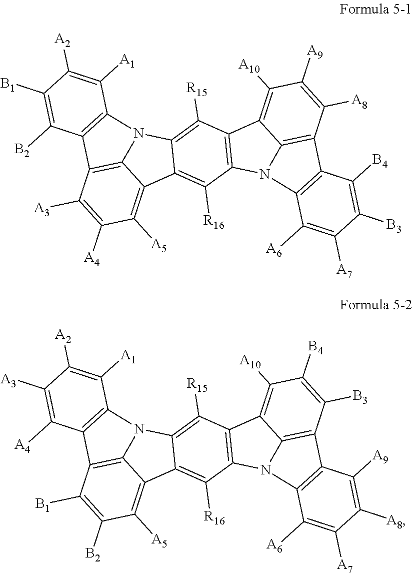
wherein, in Formula 5-1 and Formula 5-2,
A₁ to A₁₀ are each independently a hydrogen atom, a deuterium atom, a halogen atom, a cyano group, a substituted or unsubstituted silyl group, a substituted or unsubstituted amine group, a substituted or unsubstituted oxy group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and/or bonded to an adjacent group to form a ring,
B₁ and B₂ are bonded to each other to form the substituent represented by Formula 2, and B₃ and B₄ are bonded to each other to form the substituent represented by Formula 2, and
R₁₅ to R₁₆ are the same as defined in Formula 1.

5. The light emitting device of claim 4, wherein the first compound represented by Formula 1 is represented by any one from among Formula 6-1 to Formula 6-4: 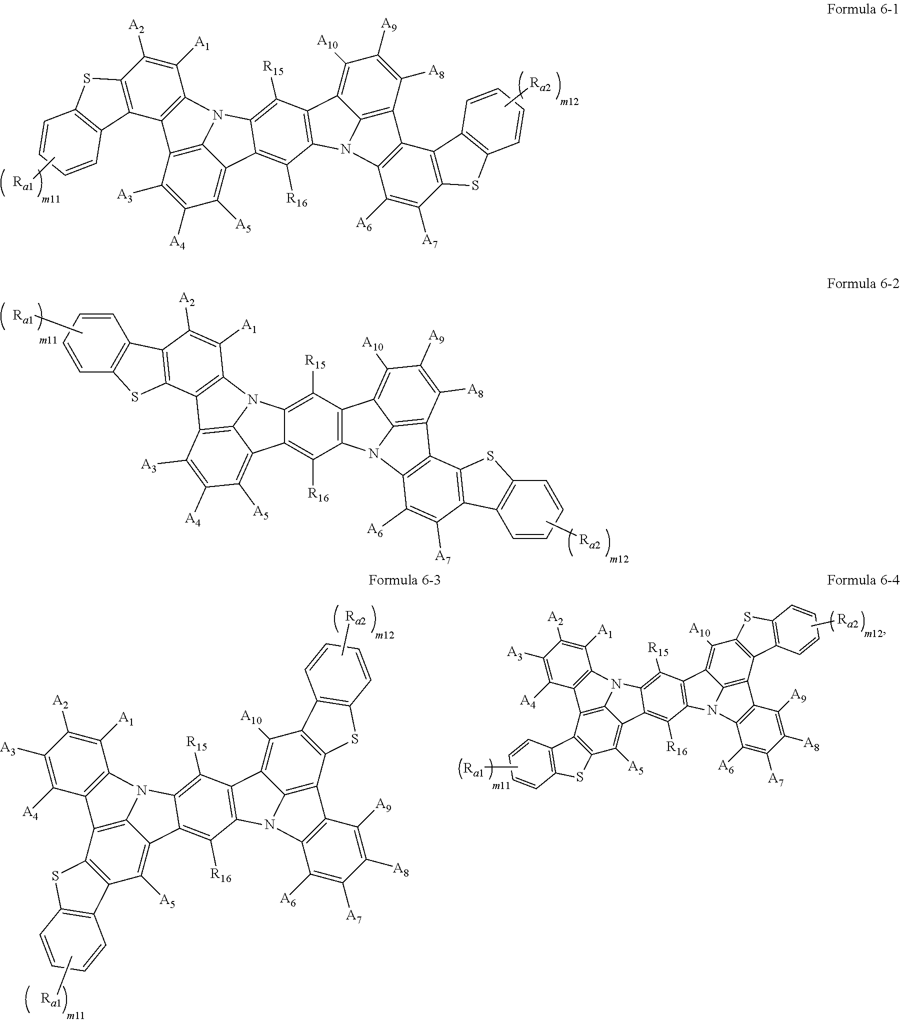
wherein, in Formula 6-1 to Formula 6-4,
Rₐ₁ and Rₐ₂ are each independently a hydrogen atom, a deuterium atom, a halogen atom, a cyano group, a substituted or unsubstituted silyl group, a substituted or unsubstituted amine group, a substituted or unsubstituted oxy group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and/or bonded to an adjacent group to form a ring,
m11 and m12 are each independently an integer of 0 to 4, and
R₁₅, R₁₆, and A₁ to A₁₀ are the same as respectively defined in Formula 1, Formula 5-1, and Formula 5-2.

6. The light emitting device of claim 4, wherein the first compound represented by Formula 1 is represented by any one from among Formula 7-1 to Formula 7-4: 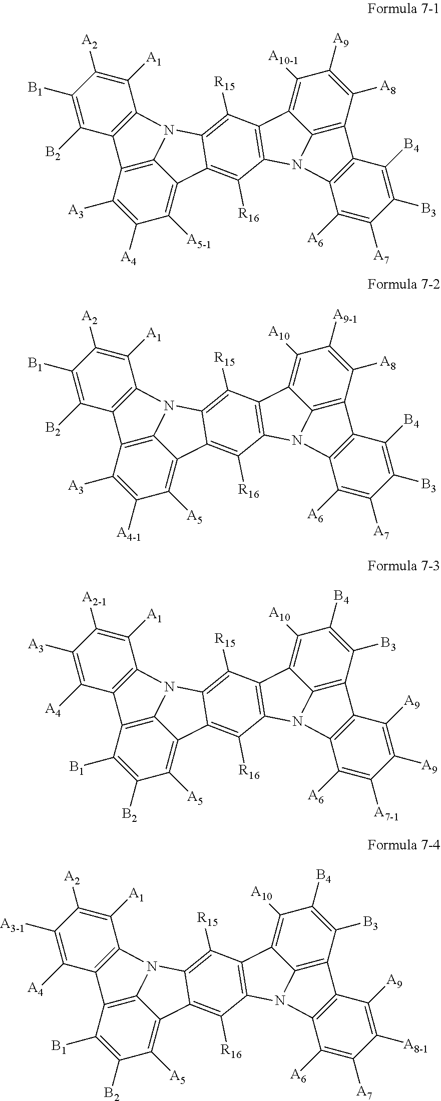
wherein, in Formula 7-1 to Formula 7-4,
A₂₋₁ to A₅₋₁ and A₇₋₁ to A₁₀₋₁ are each independently a substituted or unsubstituted aryl group having 6 to 30 ring-forming carbon atoms, or a substituted or unsubstituted heteroaryl group having 2 to 30 ring-forming carbon atoms, and
R₁₅, R₁₆, A₁ to A₁₀, and B₁ to B₄ are the same as respectively defined in Formula 1, Formula 5-1, and Formula 5-2.

7. The light emitting device of claim 6, wherein A₂₋₁ to A₅₋₁ and A₇₋₁ to A₁₀₋₁ are each independently represented by any one from among Formula 8-1 to Formula 8-4: 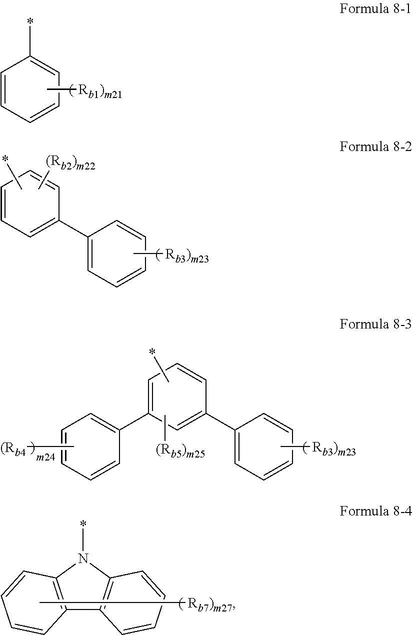
wherein, in Formula 8-1 to Formula 8-4,
R_{b1} to R_{b7} are each independently a hydrogen atom, a deuterium atom, a halogen atom, a substituted or unsubstituted alkyl group having 1 to 20 carbon atoms a substituted or unsubstituted aryl group having 6 to 30 ring-forming carbon atoms, or a substituted or unsubstituted heteroaryl group having 2 to 30 ring-forming carbon atoms,
m21, m23, m24, and m26 are each independently an integer of 0 to 5,
m22 is an integer of 0 to 4,
m25 is an integer of 0 to 3, and
m27 is an integer of 0 to 8.

8. The light emitting device of claim 1, wherein the first compound represented by Formula 1 is represented by Formula 9: 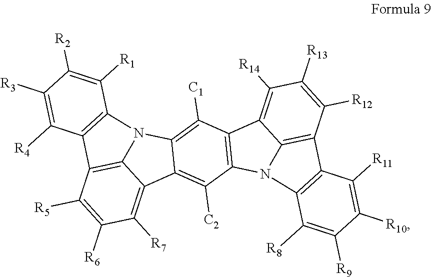
wherein, in Formula 9,
C1 and C2 are each independently a hydrogen atom or a deuterium atom, and
R₁ to R₁₄ are the same as defined in Formula 1.

9. The light emitting device of claim 1, wherein the first compound represented by Formula 1 comprises at least one from among compounds in Compound Group 1:

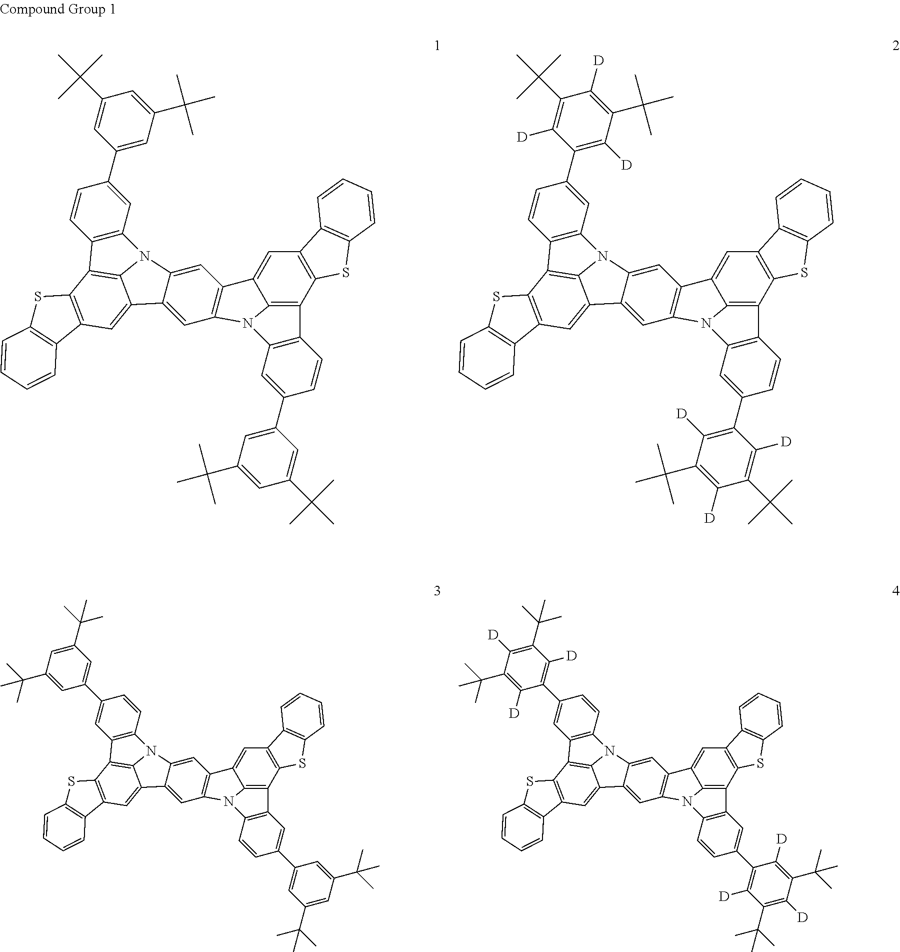

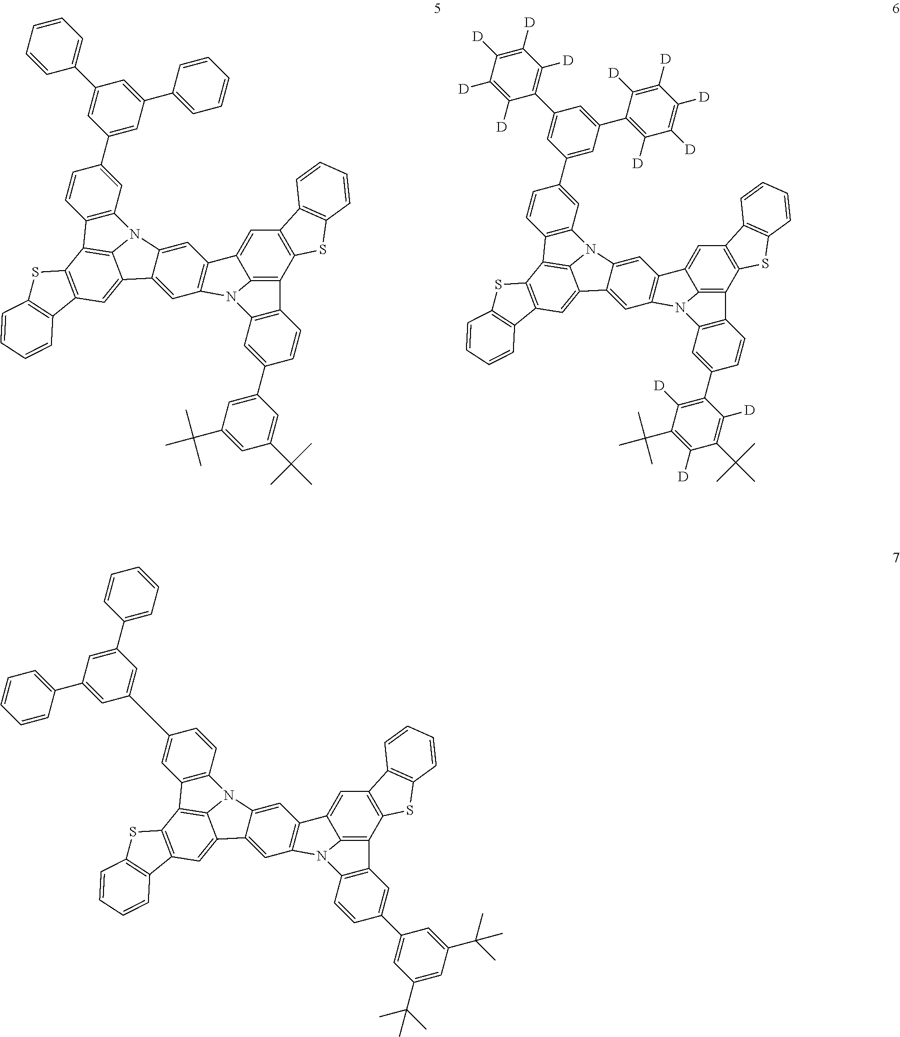 [SMILES: CC(C)(C)c1cc(-c2ccc3c4c5sc6ccccc6c5cc5c6cc7c(cc6n(c3c2)c54)c2cc3c4ccccc4sc3c3c4ccc(-c5cc(-c6ccccc6)cc(-c6ccccc6)c5)cc4n7c23)cc(C(C)(C)C)c1 ; [2H]c1c([2H])c([2H])c(-c2cc(-c3ccc4c5c6sc7ccccc7c6cc6c7cc8c(cc7n(c4c3)c65)c3cc4c5ccccc5sc4c4c5ccc(-c6c([2H])c(C(C)(C)C)c([2H])c(C(C)(C)C)c6[2H])cc5n8c34)cc(-c3c([2H])c([2H])c([2H])c([2H])c3[2H])c2)c([2H])c1[2H] ; CC(C)(C)c1cc(-c2ccc3c(c2)c2c4sc5ccccc5c4cc4c5cc6c(cc5n3c42)c2cc3c4ccccc4sc3c3c4cc(Cc5cc(-c7ccccc7)cc(-c7ccccc7)c5)ccc4n6c23)cc(C(C)(C)C)c1]

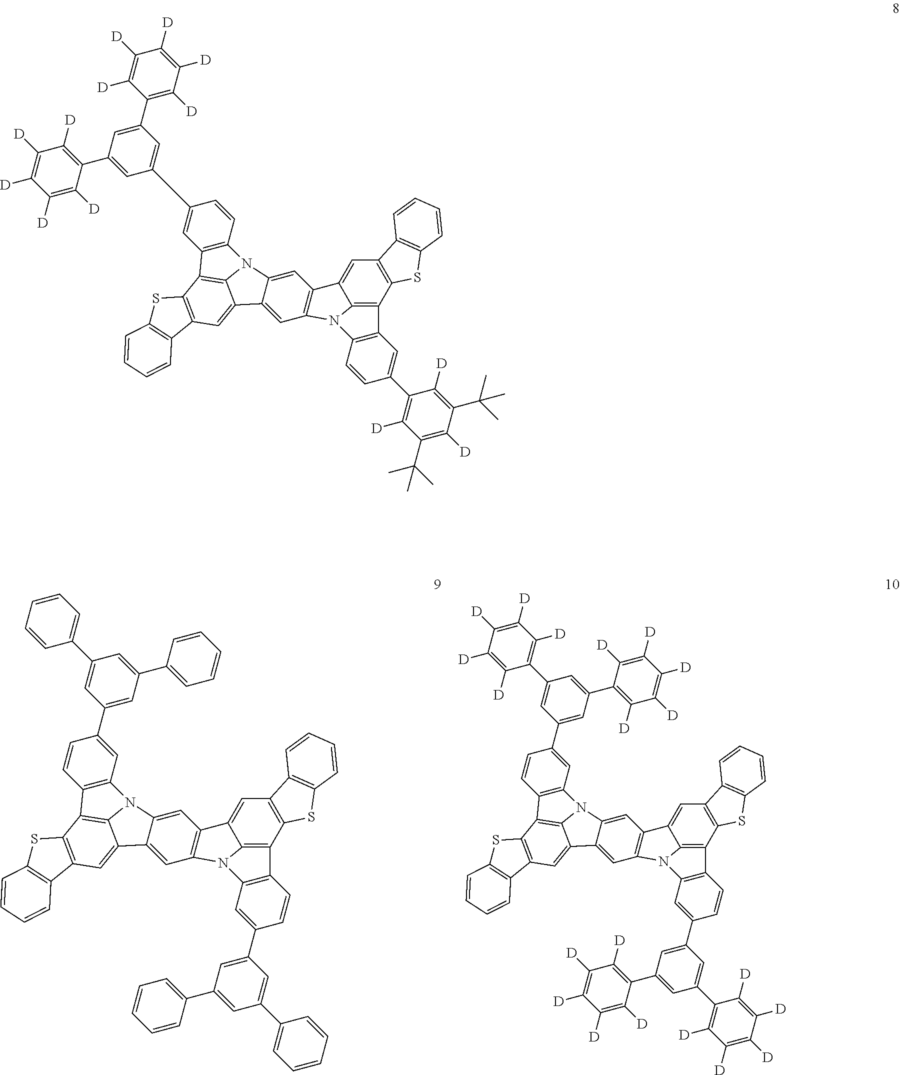

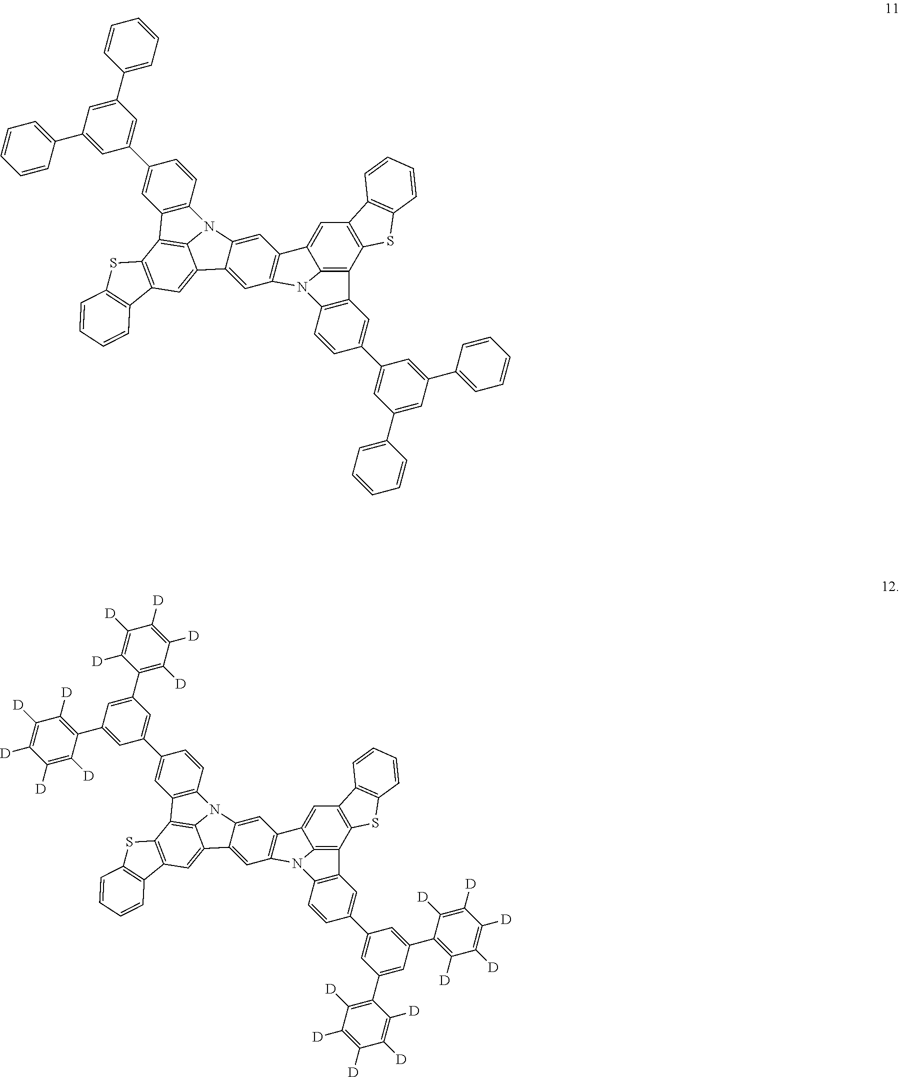 [SMILES: c1ccc(-c2cc(-c3ccccc3)cc(-c3ccc4c(c3)c3c5sc6ccccc6c5cc5c6cc7c(cc6n4c53)c3cc4c5ccccc5sc4c4c5cc(-c6cc(-c8ccccc8)cc(-c8ccccc8)c6)ccc5n7c34)c2)cc1 ; [2H]c1c([2H])c([2H])c(-c2cc(-c3ccc4c(c3)c3c5sc6ccccc6c5cc5c6cc7c(cc6n4c53)c3cc4c5ccccc5sc4c4c5cc(-c6cc(-c8c([2H])c([2H])c([2H])c([2H])c8[2H])cc(-c8c([2H])c([2H])c([2H])c([2H])c8[2H])c6)ccc5n7c34)cc(-c3c([2H])c([2H])c([2H])c([2H])c3[2H])c2)c([2H])c1[2H]]

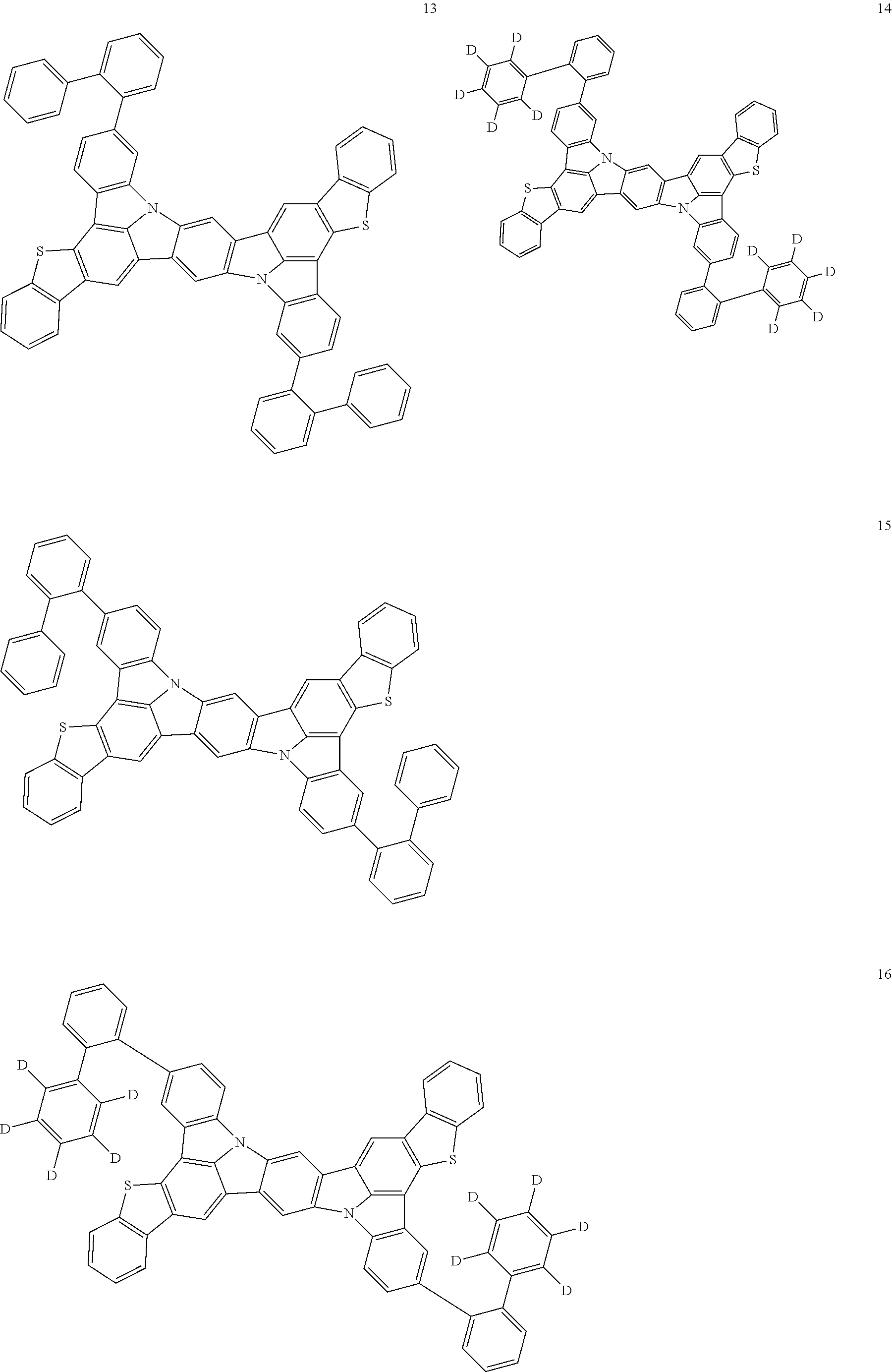

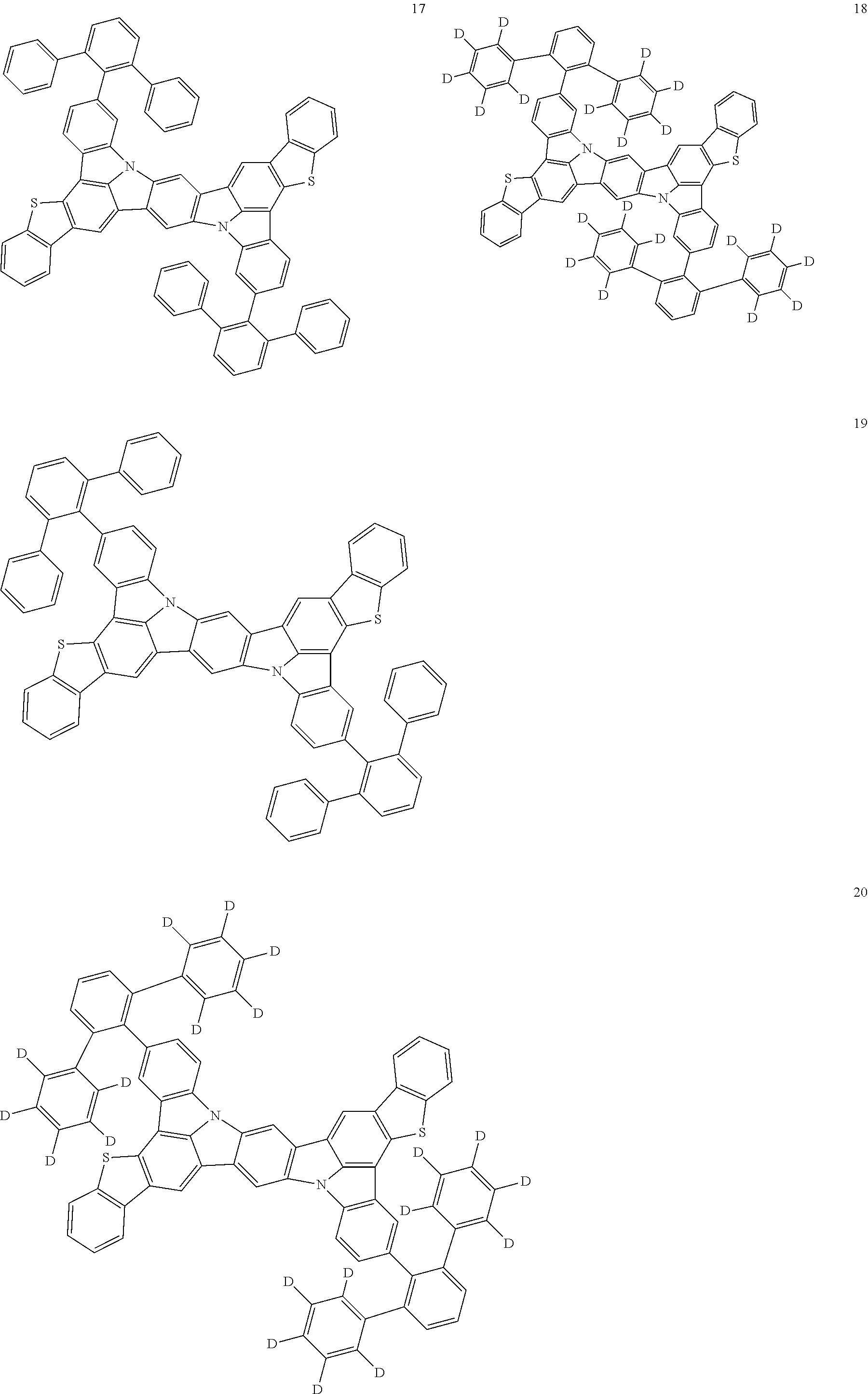

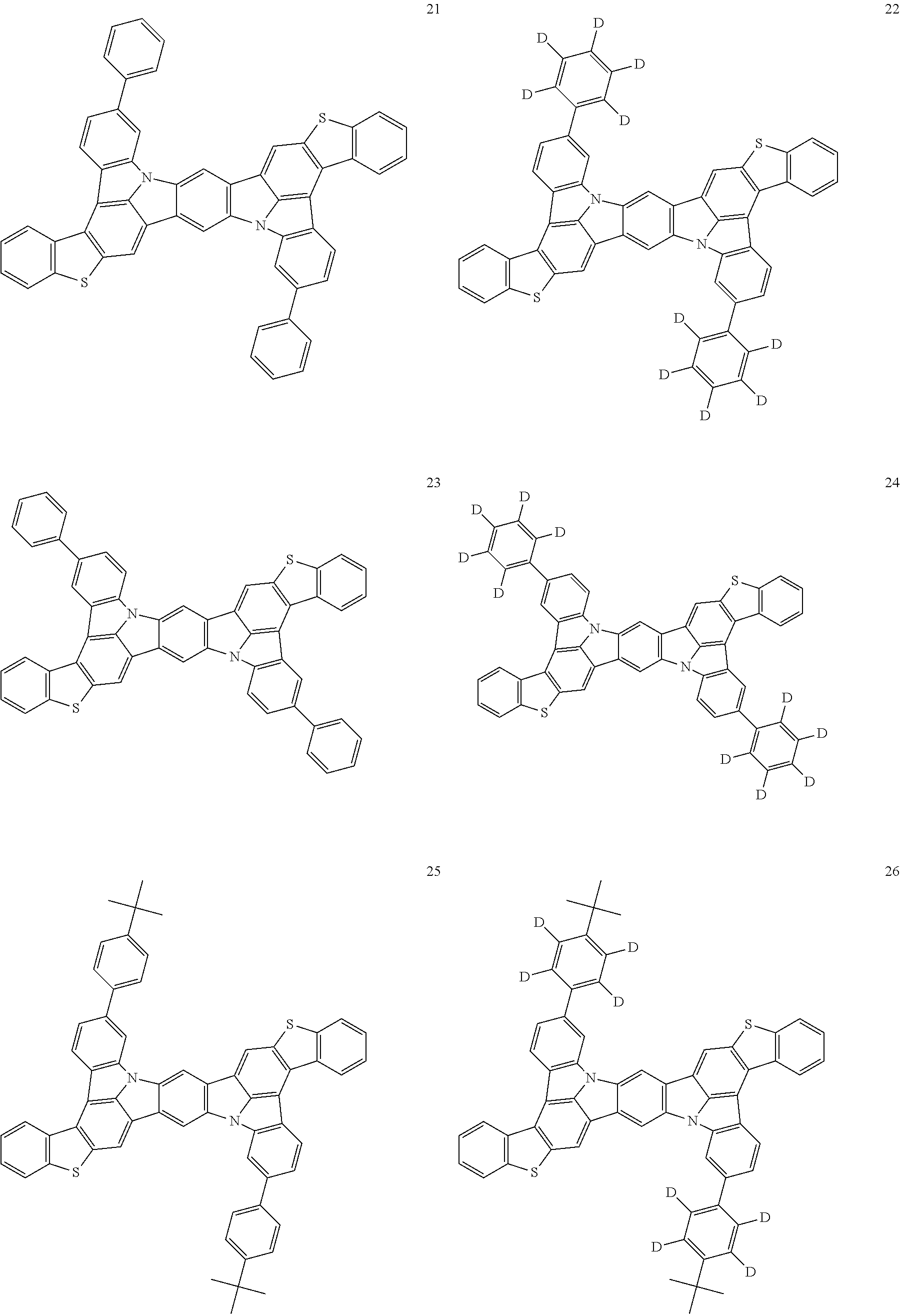

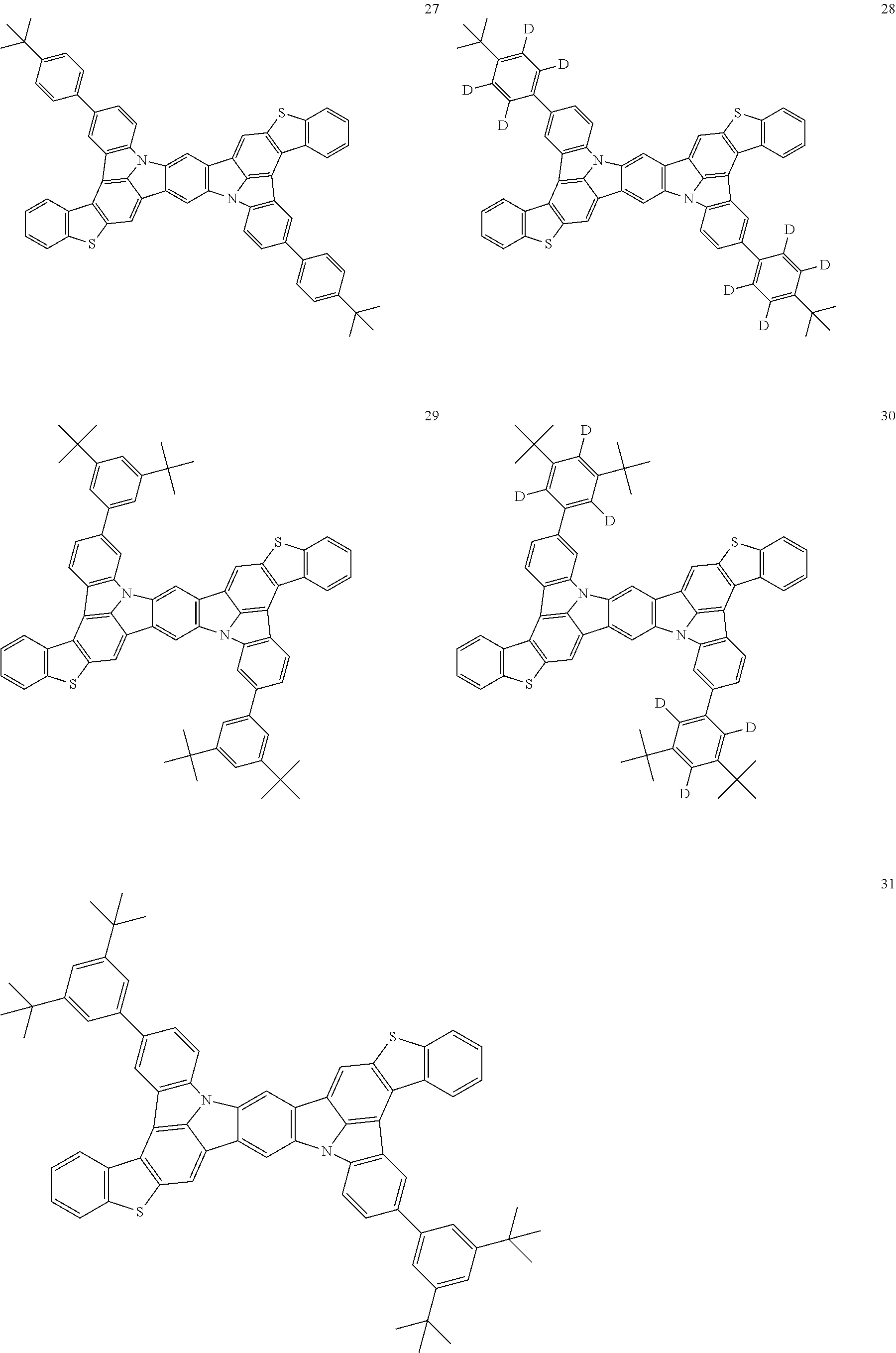

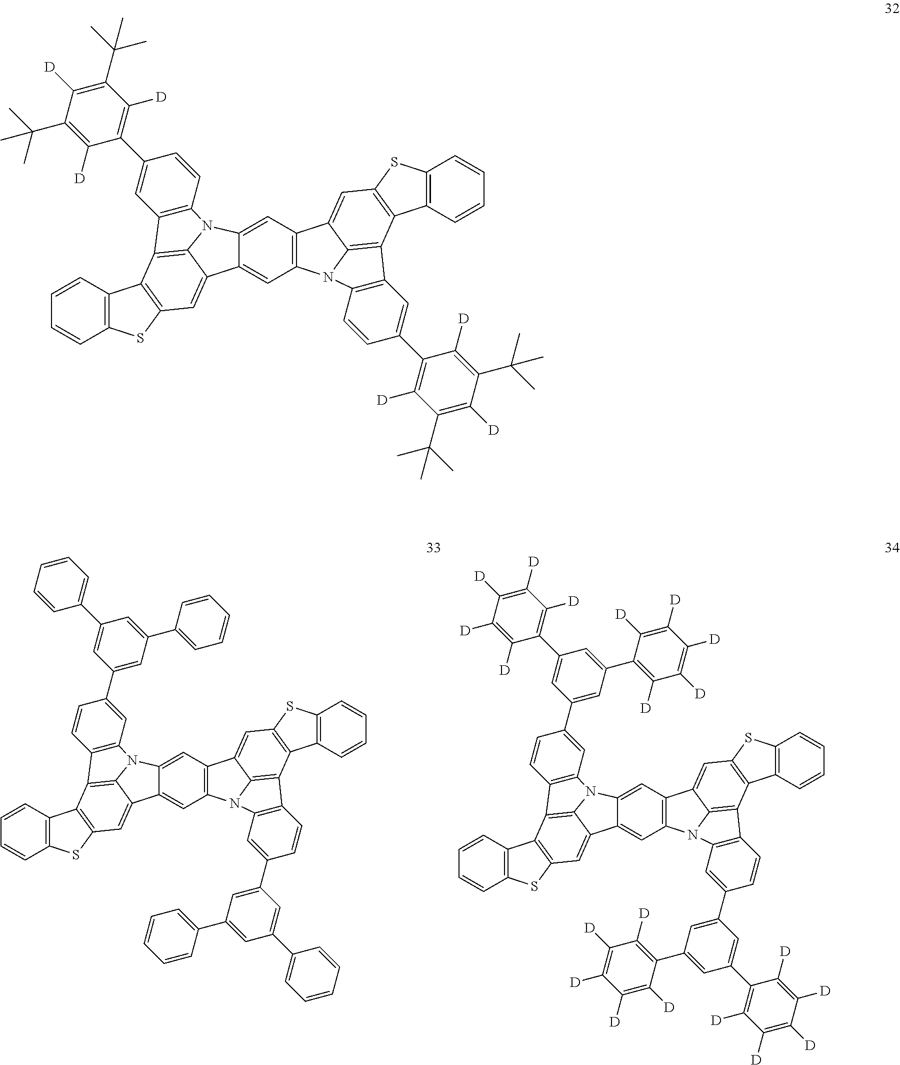

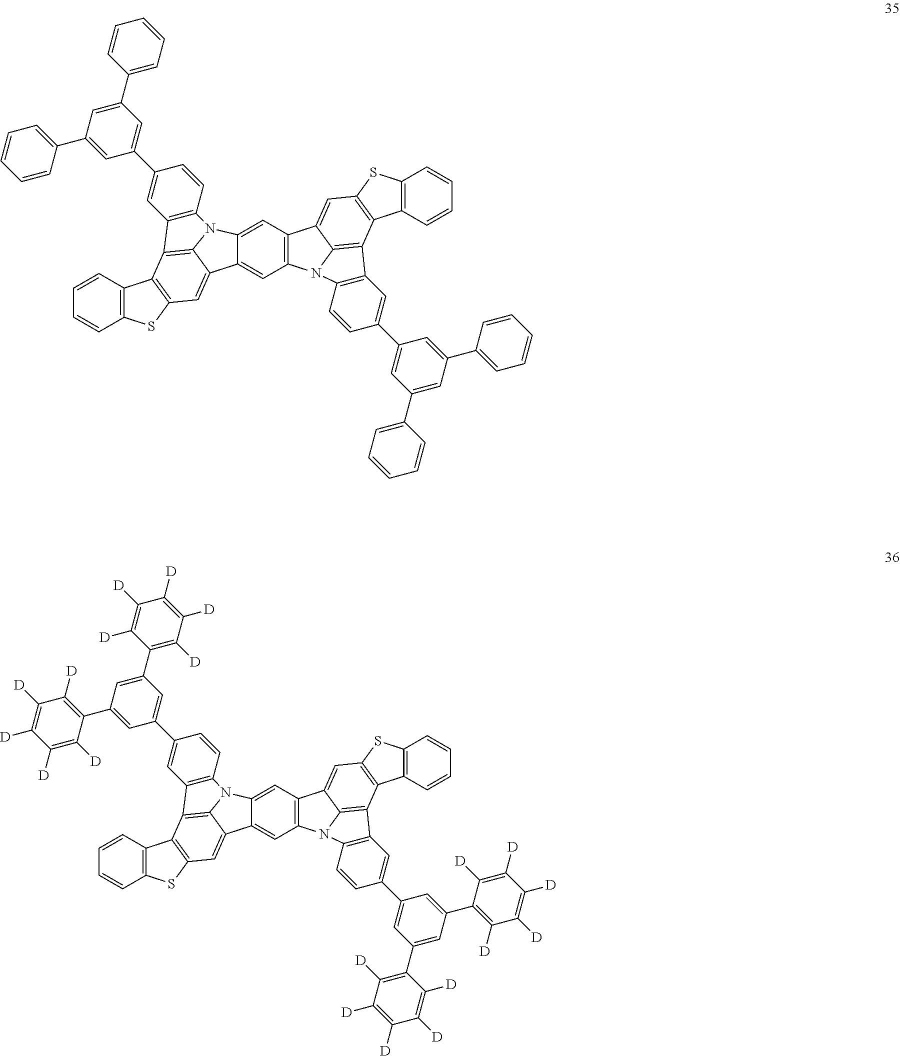 [SMILES: c1ccc(-c2cc(-c3ccccc3)cc(-c3ccc4c(c3)c3c5c(cc6c7cc8c(cc7n4c63)c3cc4sc6ccccc6c4c4c6cc(-c7cc(-c9ccccc9)cc(-c9ccccc9)c7)ccc6n8c34)sc3ccccc35)c2)cc1 ; [2H]c1c([2H])c([2H])c(-c2cc(-c3ccc4c(c3)c3c5c(cc6c7cc8c(cc7n4c63)c3cc4sc6ccccc6c4c4c6cc(-c7cc(-c9c([2H])c([2H])c([2H])c([2H])c9[2H])cc(-c9c([2H])c([2H])c([2H])c([2H])c9[2H])c7)ccc6n8c34)sc3ccccc35)cc(-c3c([2H])c([2H])c([2H])c([2H])c3[2H])c2)c([2H])c1[2H]]

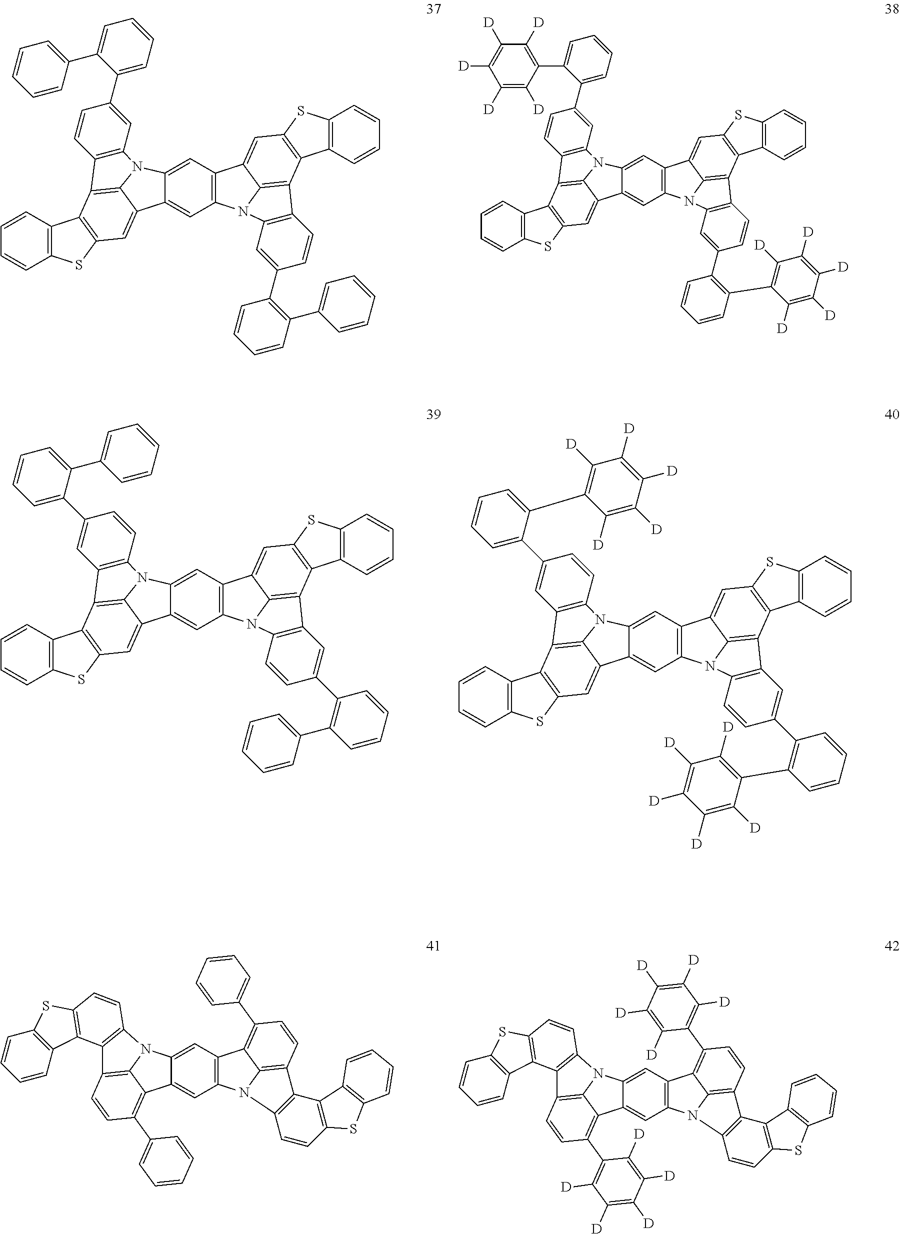

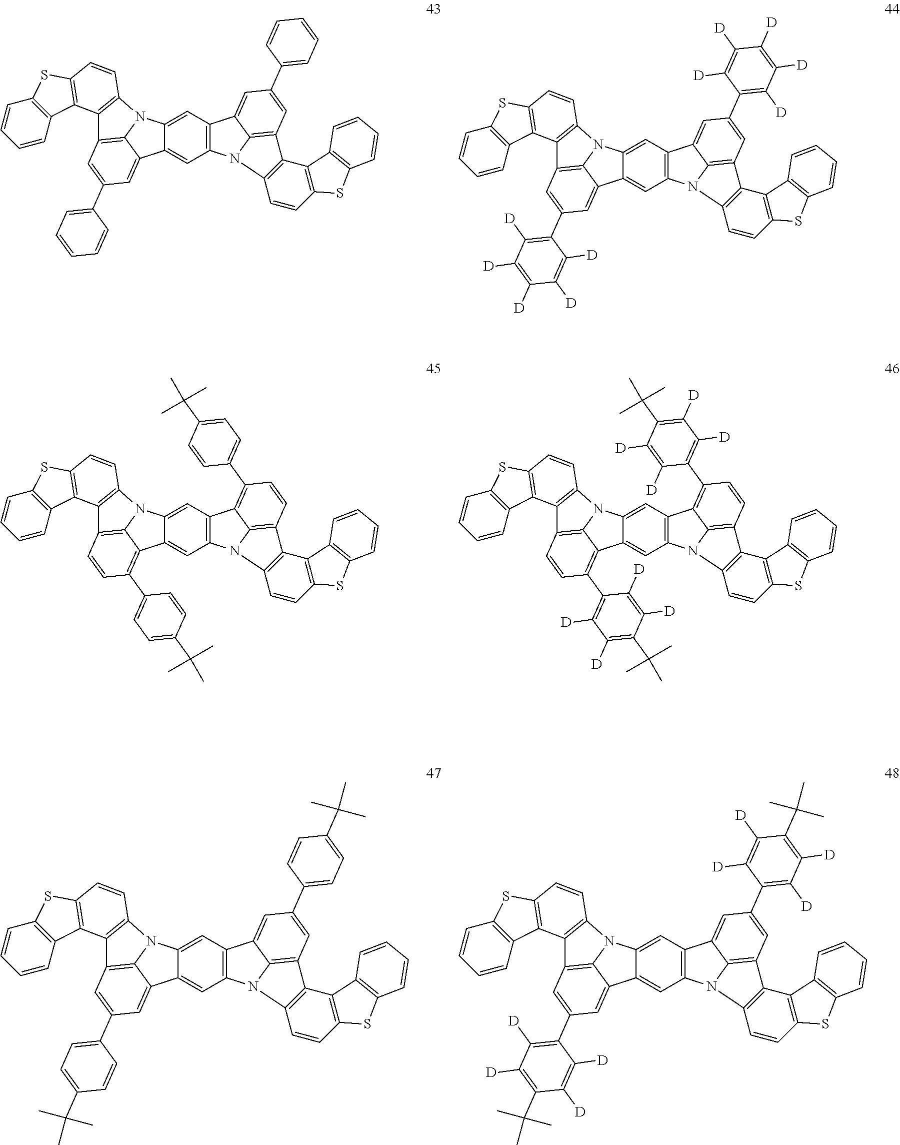

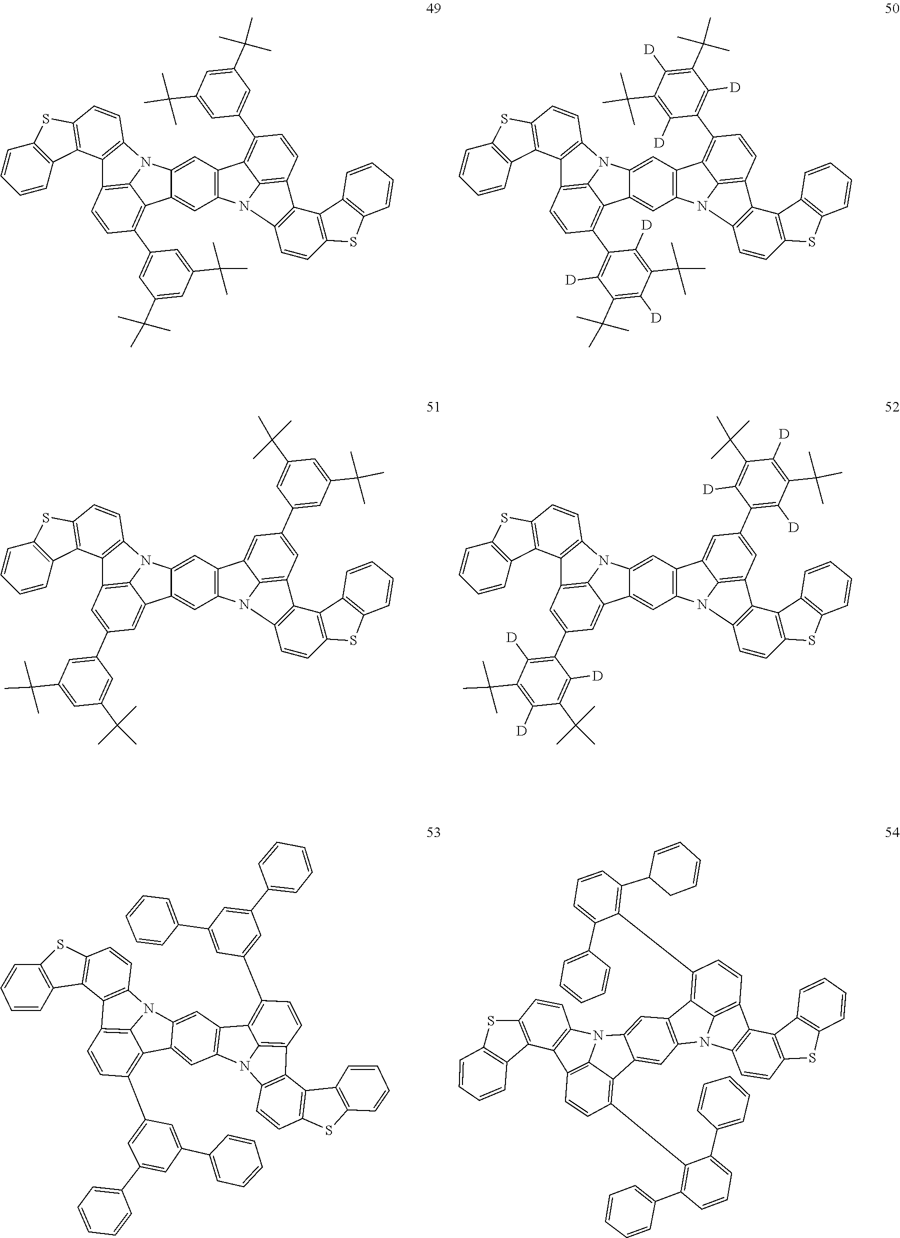

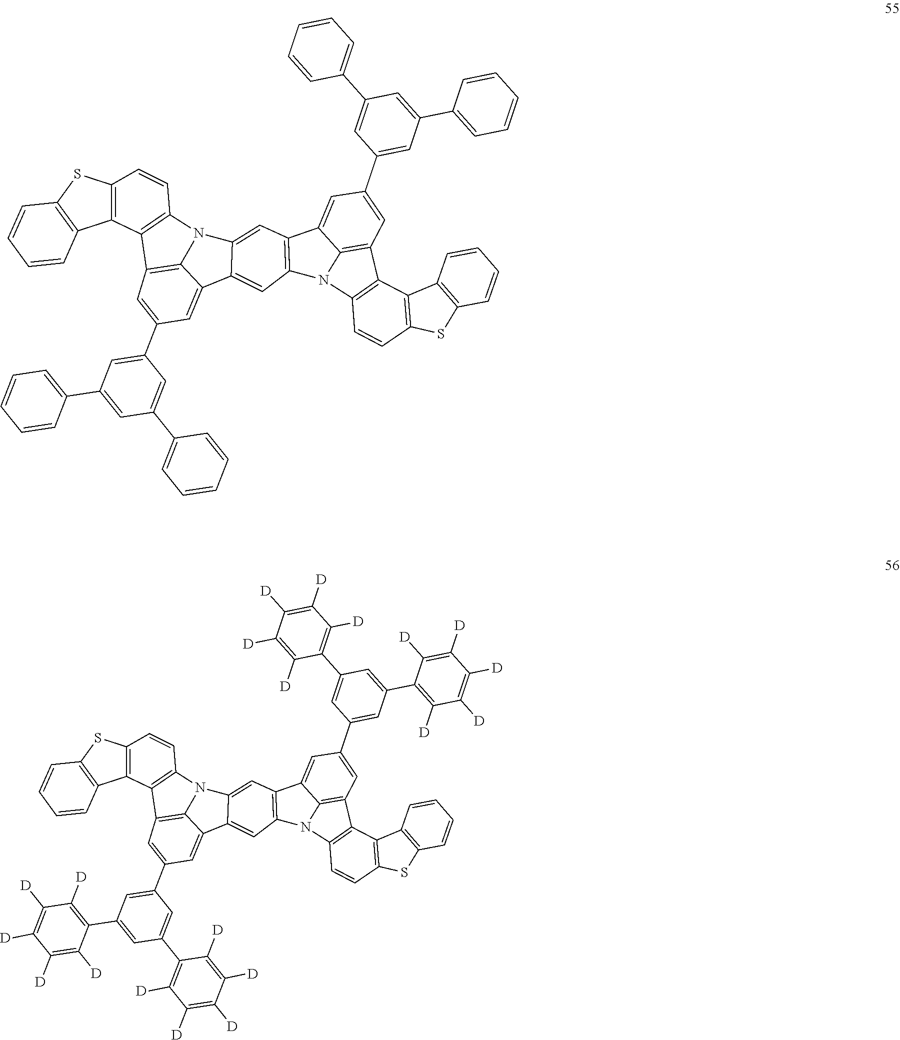 [SMILES: c1ccc(-c2cc(-c3ccccc3)cc(-c3cc4c5cc6c(cc5n5c7ccc8sc9ccccc9c8c7c(c3)c45)c3cc(-c4cc(-c5ccccc5)cc(-c5ccccc5)c4)cc4c5c7c(ccc5n6c34)sc3ccccc37)c2)cc1 ; [2H]c1c([2H])c([2H])c(-c2cc(-c3cc4c5cc6c(cc5n5c7ccc8sc9ccccc9c8c7c(c3)c45)c3cc(-c4cc(-c5c([2H])c([2H])c([2H])c([2H])c5[2H])cc(-c5c([2H])c([2H])c([2H])c([2H])c5[2H])c4)cc4c5c7c(ccc5n6c34)sc3ccccc37)cc(-c3c([2H])c([2H])c([2H])c([2H])c3[2H])c2)c([2H])c1[2H]]

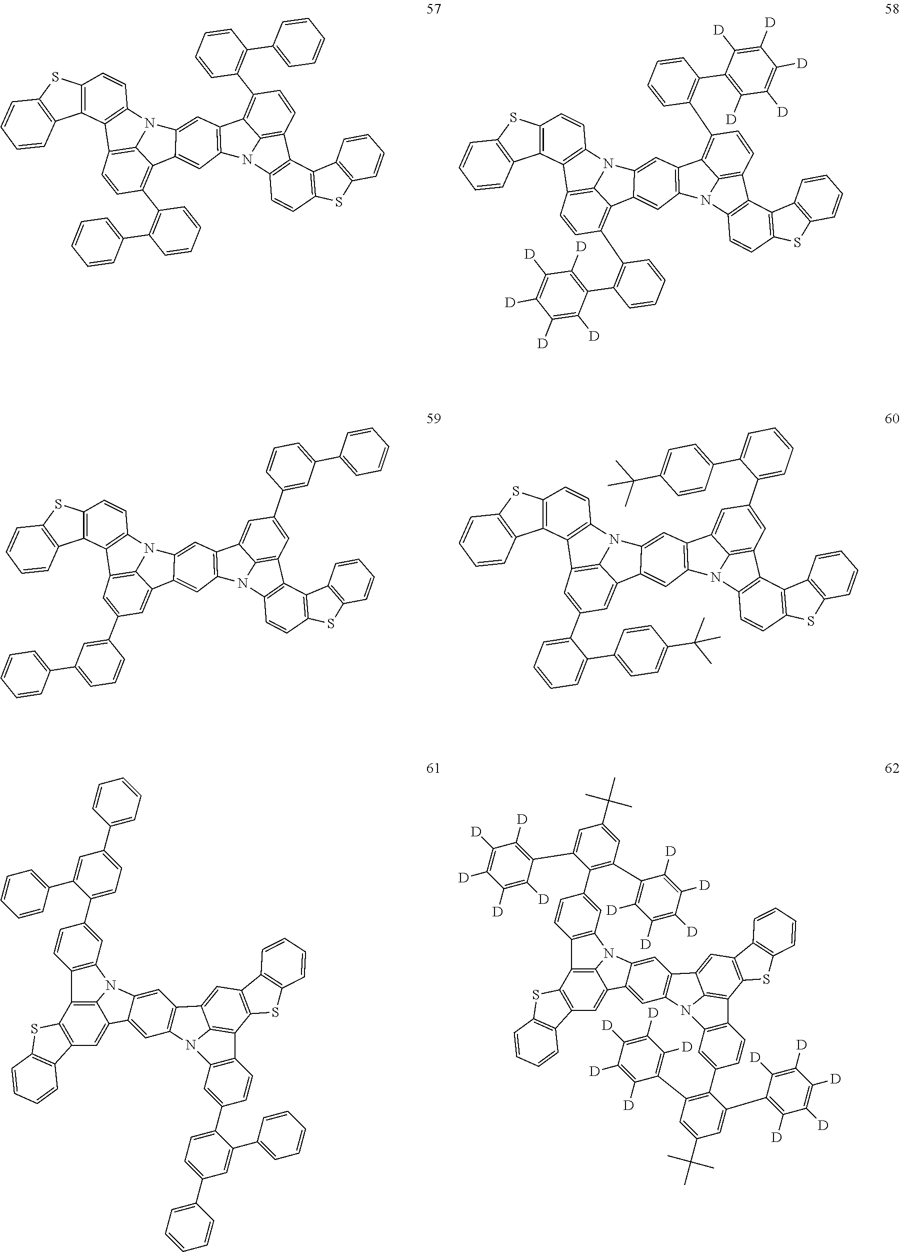

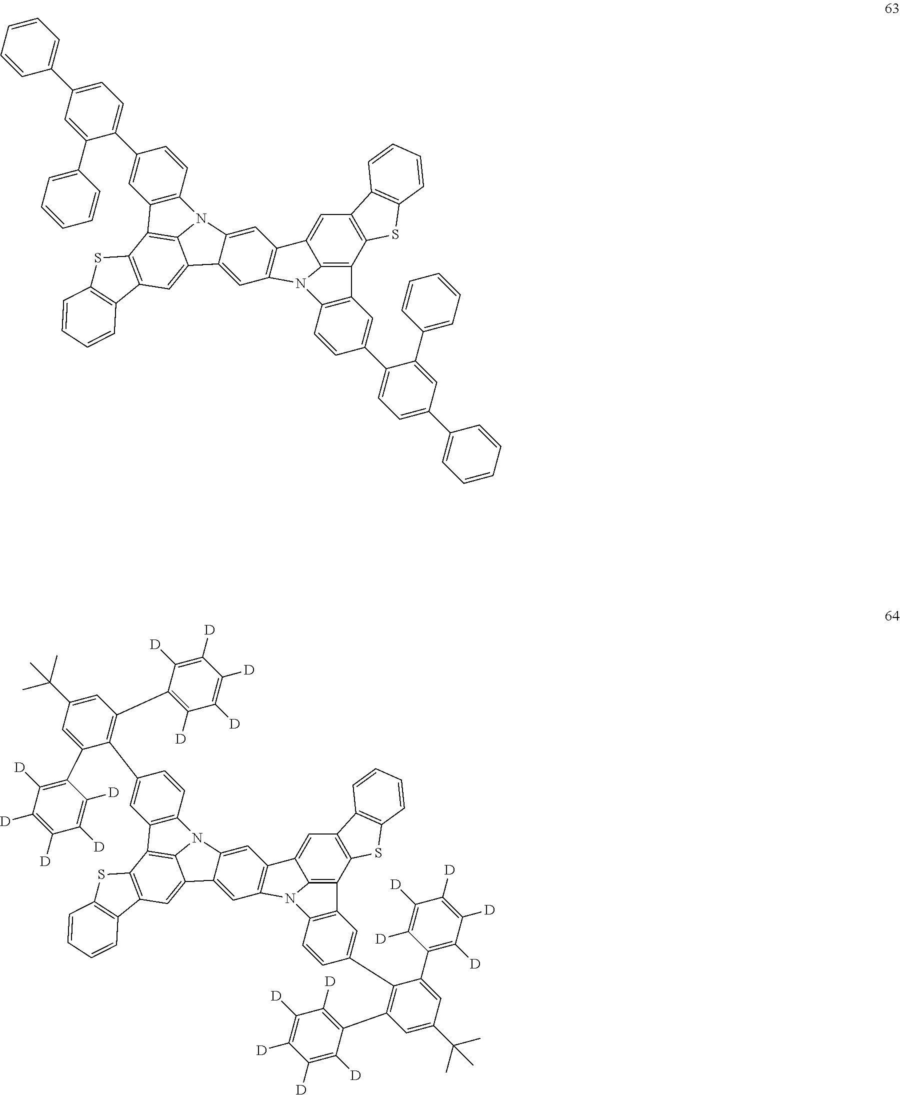 [SMILES: c1ccc(-c2ccc(-c3ccc4c(c3)c3c5sc6ccccc6c5cc5c6cc7c(cc6n4c53)c3cc4c5ccccc5sc4c4c5cc(-c6ccc(-c8ccccc8)cc6-c6ccccc6)ccc5n7c34)c(-c3ccccc3)c2)cc1 ; [2H]c1c([2H])c([2H])c(-c2cc(C(C)(C)C)cc(-c3c([2H])c([2H])c([2H])c([2H])c3[2H])c2-c2ccc3c(c2)c2c4sc5ccccc5c4cc4c5cc6c(cc5n3c42)c2cc3c4ccccc4sc3c3c4cc(-c5c(-c7c([2H])c([2H])c([2H])c([2H])c7[2H])cc(C(C)(C)C)cc5-c5c([2H])c([2H])c([2H])c([2H])c5[2H])ccc4n6c23)c([2H])c1[2H]]

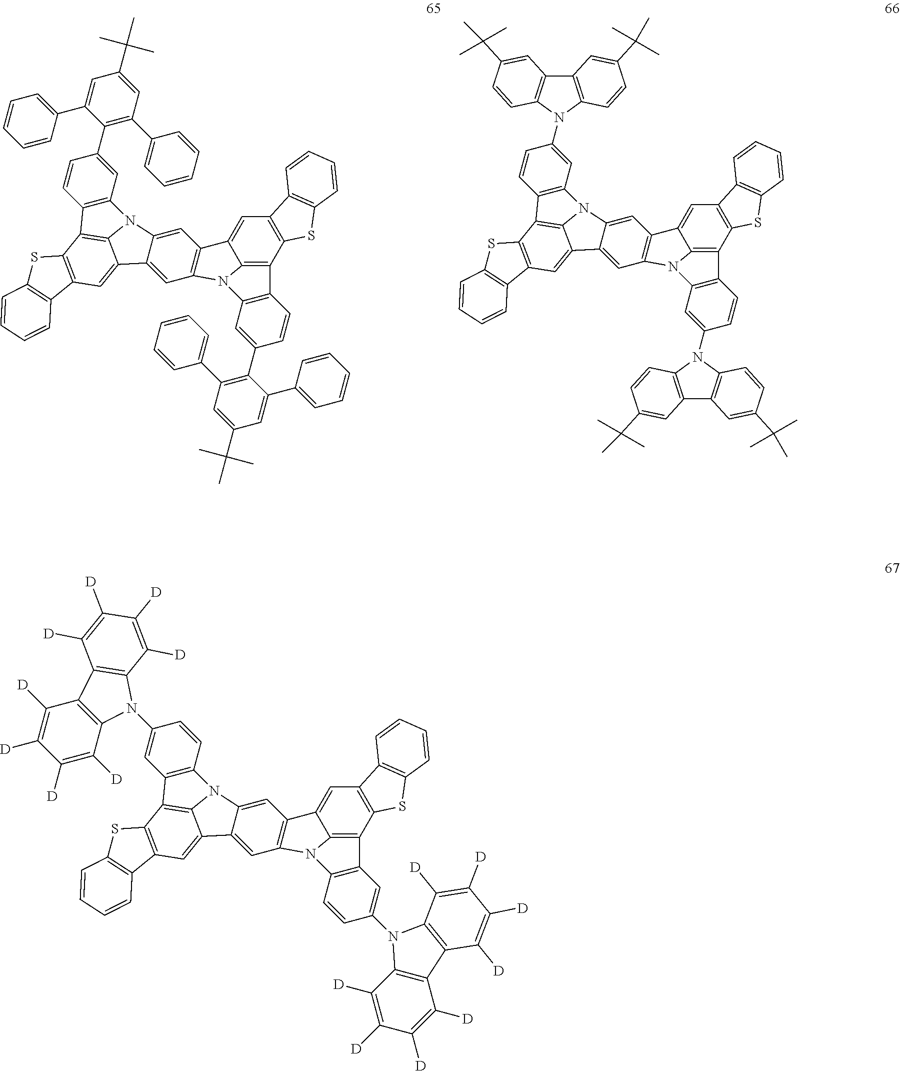

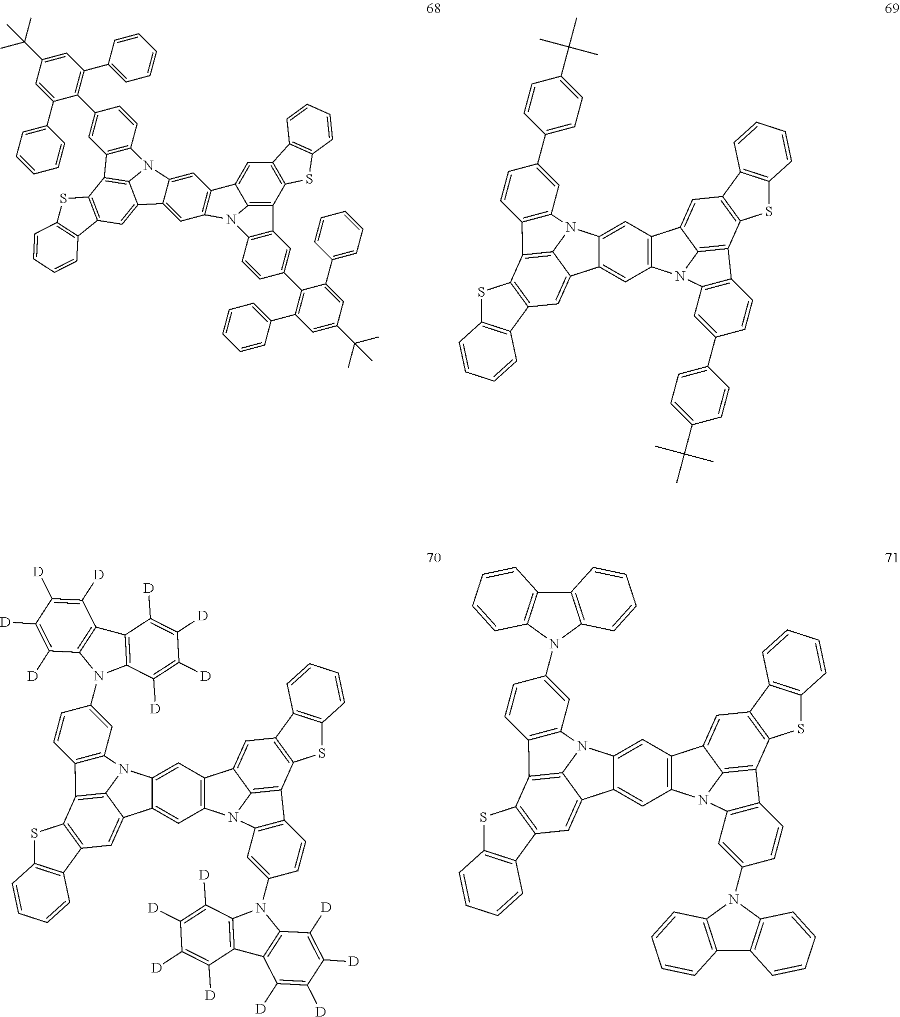

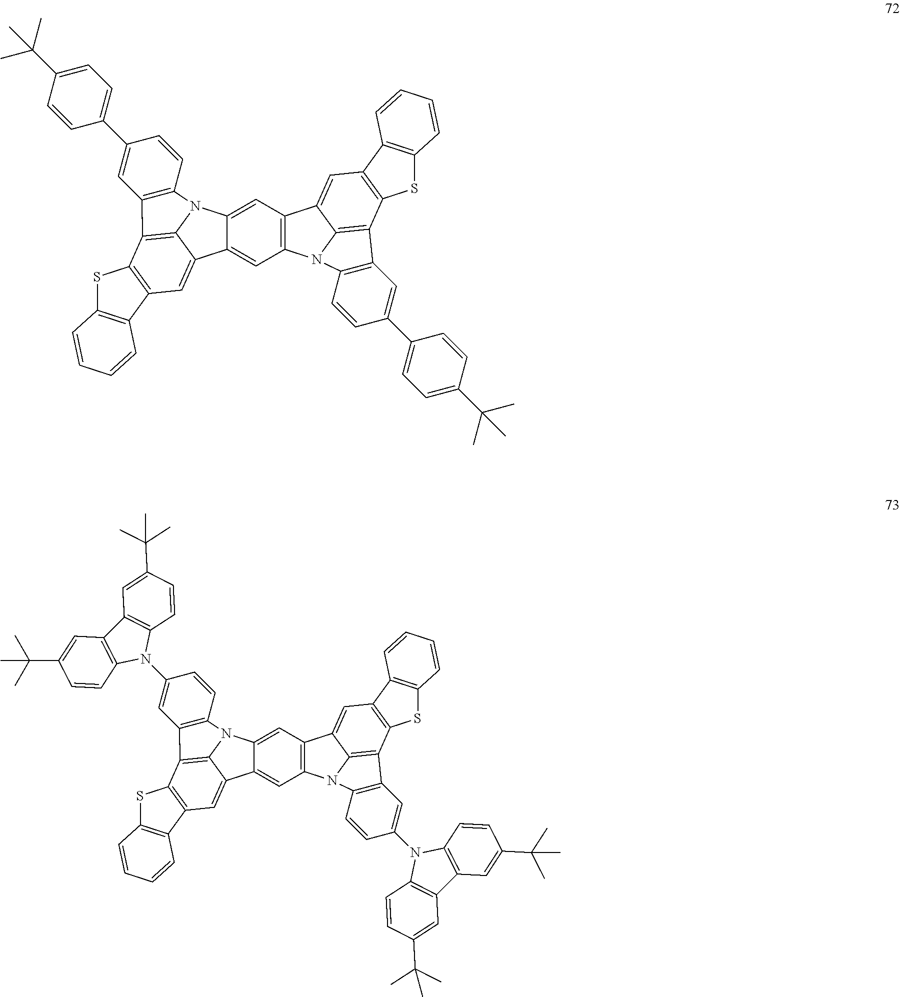

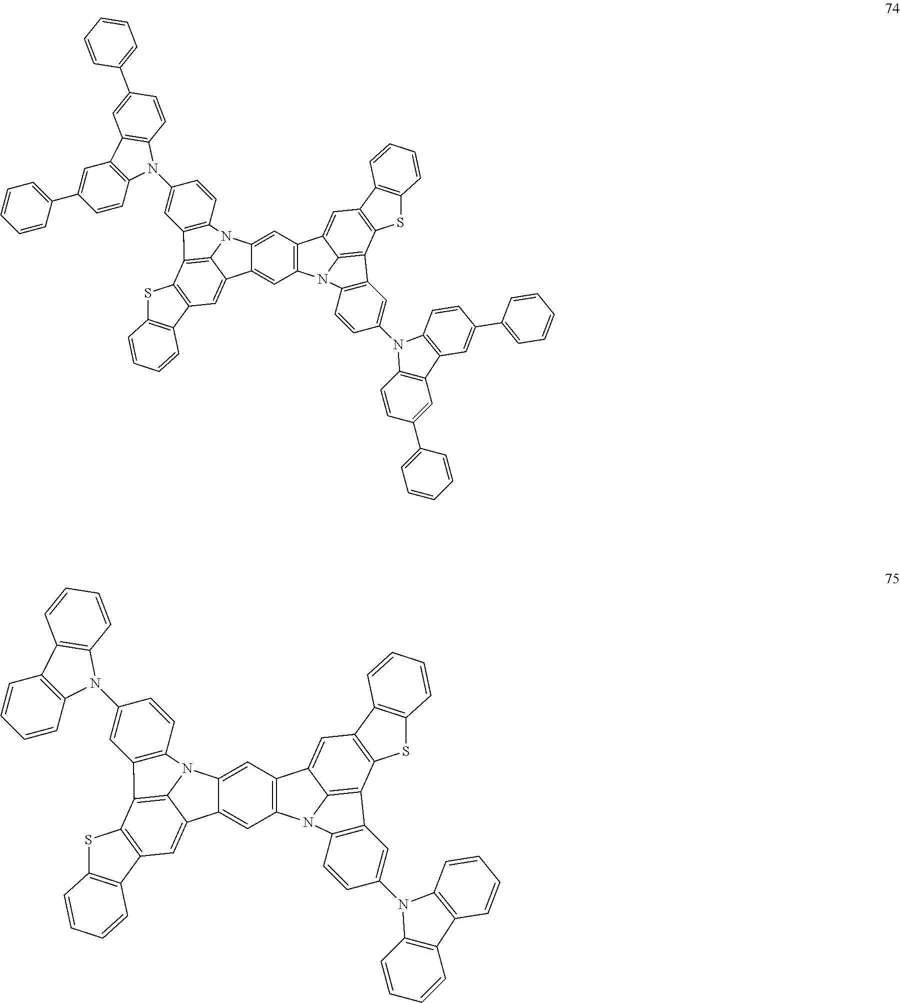 [SMILES: c1ccc(-c2ccc3c(c2)c2cc(-c4ccccc4)ccc2n3-c2ccc3c(c2)c2c4sc5ccccc5c4cc4c5cc6c(cc5n3c42)c2cc3c4ccccc4sc3c3c4cc(-n5c7ccc(-c8ccccc8)cc7c7cc(-c8ccccc8)ccc75)ccc4n6c23)cc1 ; c1ccc2c(c1)sc1c2cc2c3cc4c(cc3n3c5ccc(-n6c7ccccc7c7ccccc76)cc5c1c23)c1cc2c3ccccc3sc2c2c3cc(-n5c6ccccc6c6ccccc65)ccc3n4c12]

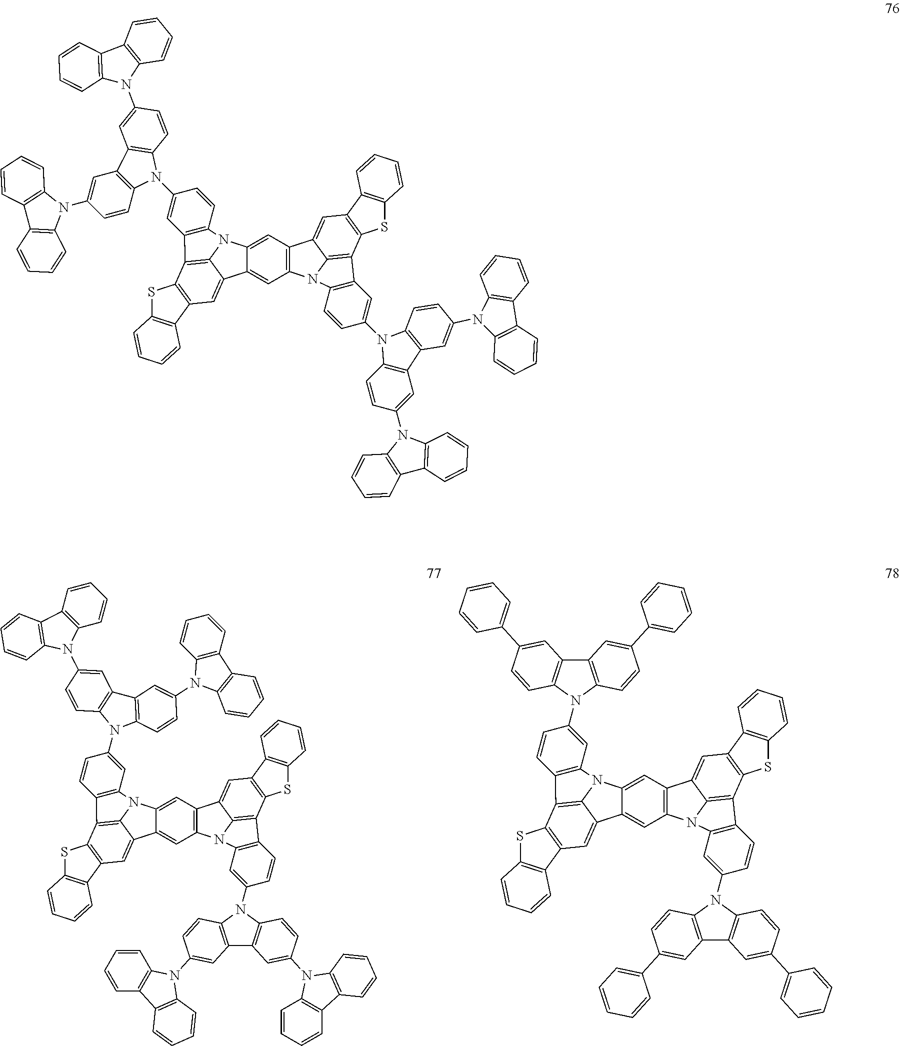

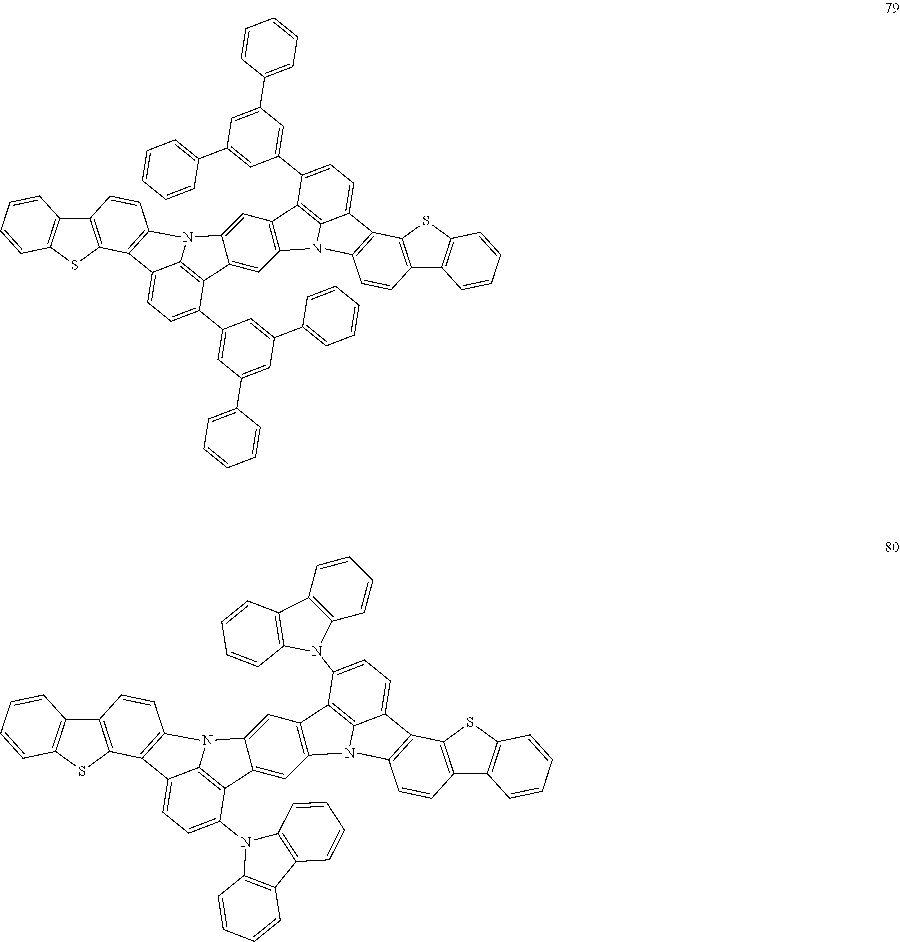 [SMILES: c1ccc(-c2cc(-c3ccccc3)cc(-c3ccc4c5c6sc7ccccc7c6ccc5n5c6cc7c8c(-c9cc(-c%10ccccc%10)cc(-c%10ccccc%10)c9)ccc9c%10c%11sc%12ccccc%12c%11ccc%10n(c7cc6c3c45)c98)c2)cc1 ; c1ccc2c(c1)sc1c2ccc2c1c1ccc(-n3c4ccccc4c4ccccc43)c3c4cc5c(cc4n2c13)c1c(-n2c3ccccc3c3ccccc32)ccc2c3c4sc6ccccc6c4ccc3n5c21]

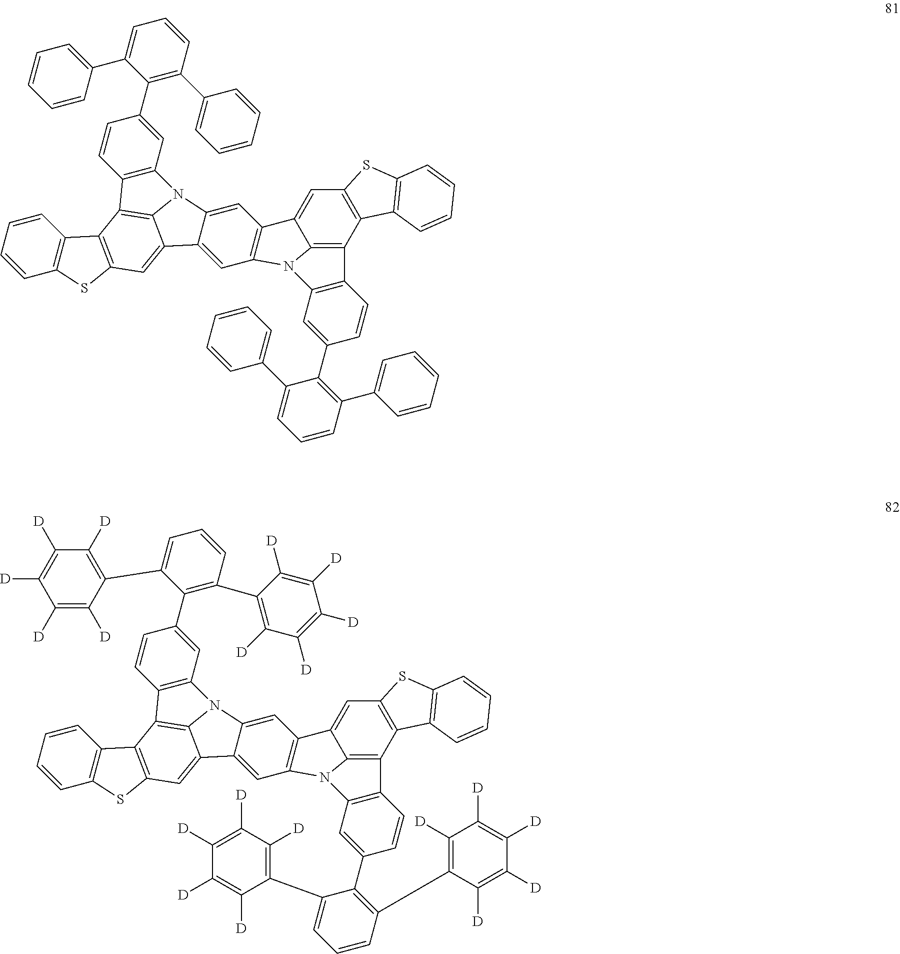 [SMILES: c1ccc(-c2cccc(-c3ccccc3)c2-c2ccc3c4c5c(cc6c7cc8c(cc7n(c3c2)c64)c2cc3sc4ccccc4c3c3c4ccc(-c6c(-c7ccccc7)cccc6-c6ccccc6)cc4n8c23)sc2ccccc25)cc1 ; [2H]c1c([2H])c([2H])c(-c2cccc(-c3c([2H])c([2H])c([2H])c([2H])c3[2H])c2-c2ccc3c4c5c(cc6c7cc8c(cc7n(c3c2)c64)c2cc3sc4ccccc4c3c3c4ccc(-c6c(-c7c([2H])c([2H])c([2H])c([2H])c7[2H])cccc6-c6c([2H])c([2H])c([2H])c([2H])c6[2H])cc4n8c23)sc2ccccc25)c([2H])c1[2H]]

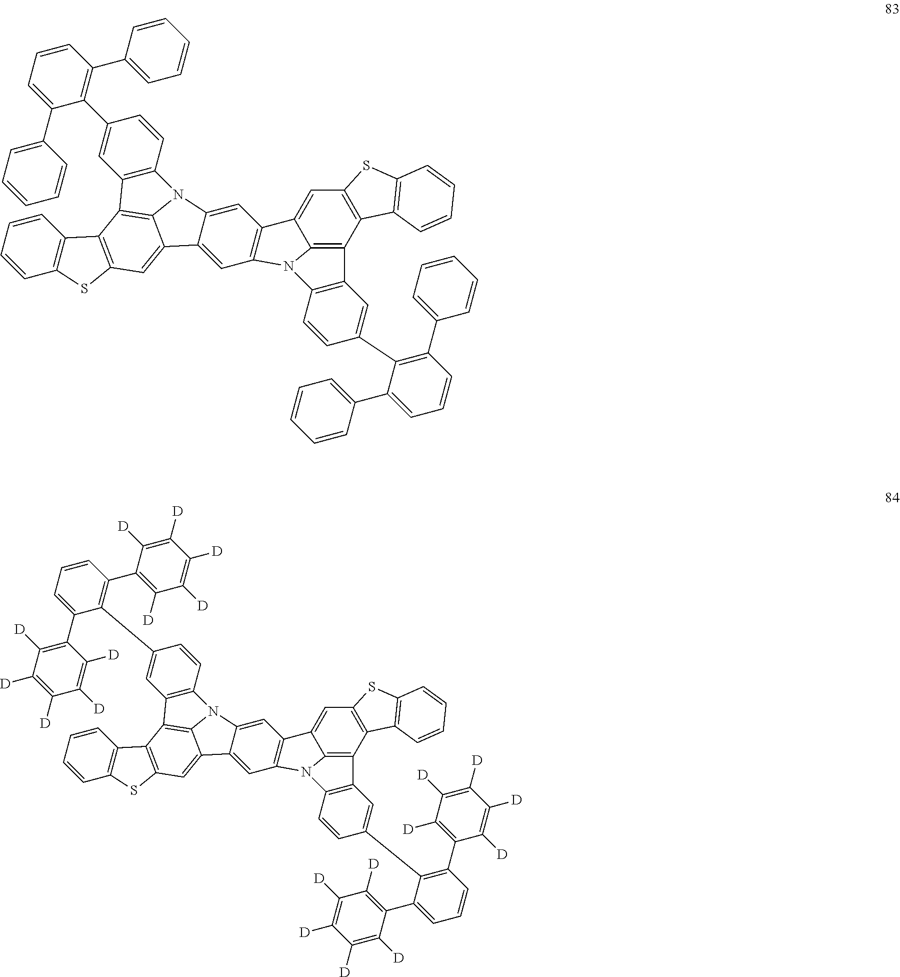 [SMILES: c1ccc(-c2cccc(-c3ccccc3)c2-c2ccc3c(c2)c2c4c(cc5c6cc7c(cc6n3c52)c2cc3sc5ccccc5c3c3c5cc(-c6c(-c8ccccc8)cccc6-c6ccccc6)ccc5n7c23)sc2ccccc24)cc1 ; [2H]c1c([2H])c([2H])c(-c2cccc(-c3c([2H])c([2H])c([2H])c([2H])c3[2H])c2-c2ccc3c(c2)c2c4c(cc5c6cc7c(cc6n3c52)c2cc3sc5ccccc5c3c3c5cc(-c6c(-c8c([2H])c([2H])c([2H])c([2H])c8[2H])cccc6-c6c([2H])c([2H])c([2H])c([2H])c6[2H])ccc5n7c23)sc2ccccc24)c([2H])c1[2H]]

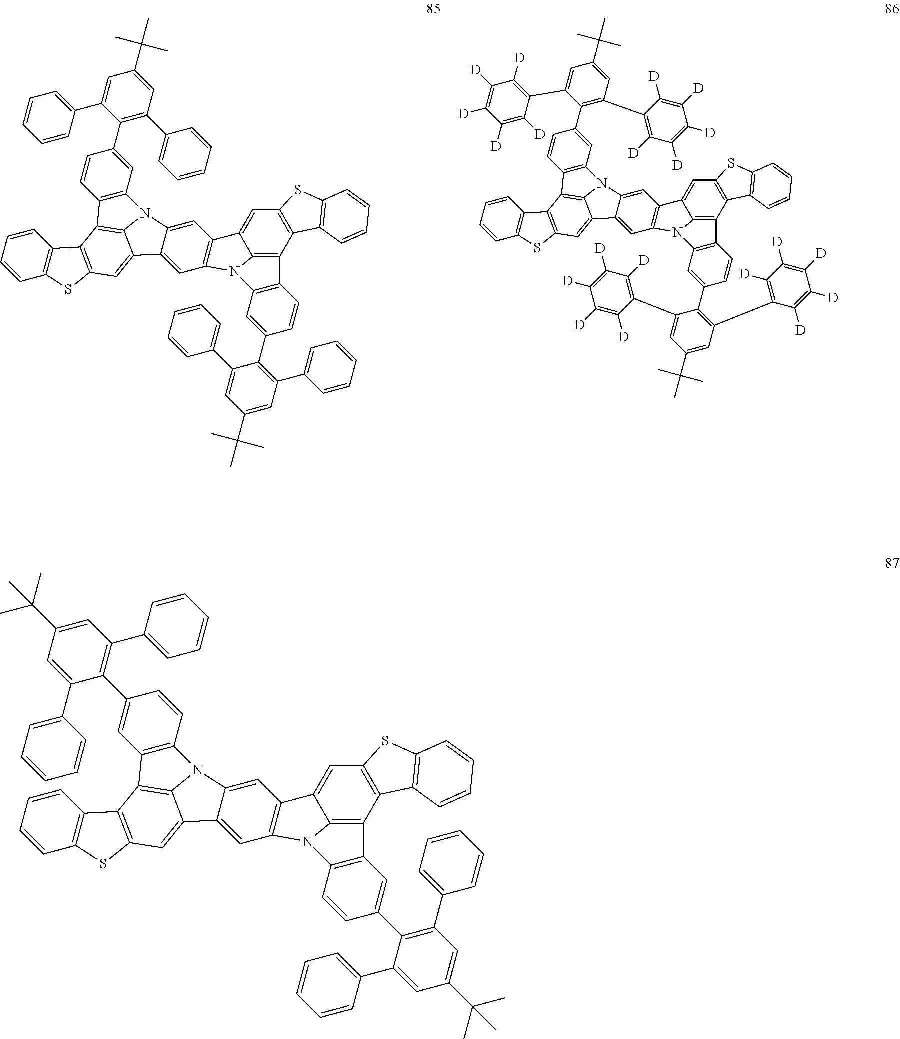

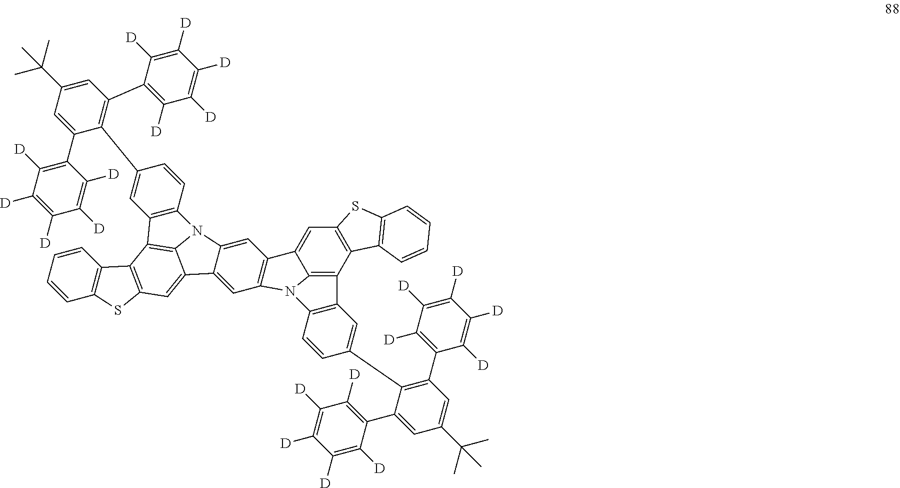 [SMILES: [2H]c1c([2H])c([2H])c(-c2cc(C(C)(C)C)cc(-c3c([2H])c([2H])c([2H])c([2H])c3[2H])c2-c2ccc3c(c2)c2c4c(cc5c6cc7c(cc6n3c52)c2cc3sc5ccccc5c3c3c5cc(-c6c(-c8c([2H])c([2H])c([2H])c([2H])c8[2H])cc(C(C)(C)C)cc6-c6c([2H])c([2H])c([2H])c([2H])c6[2H])ccc5n7c23)sc2ccccc24)c([2H])c1[2H]]

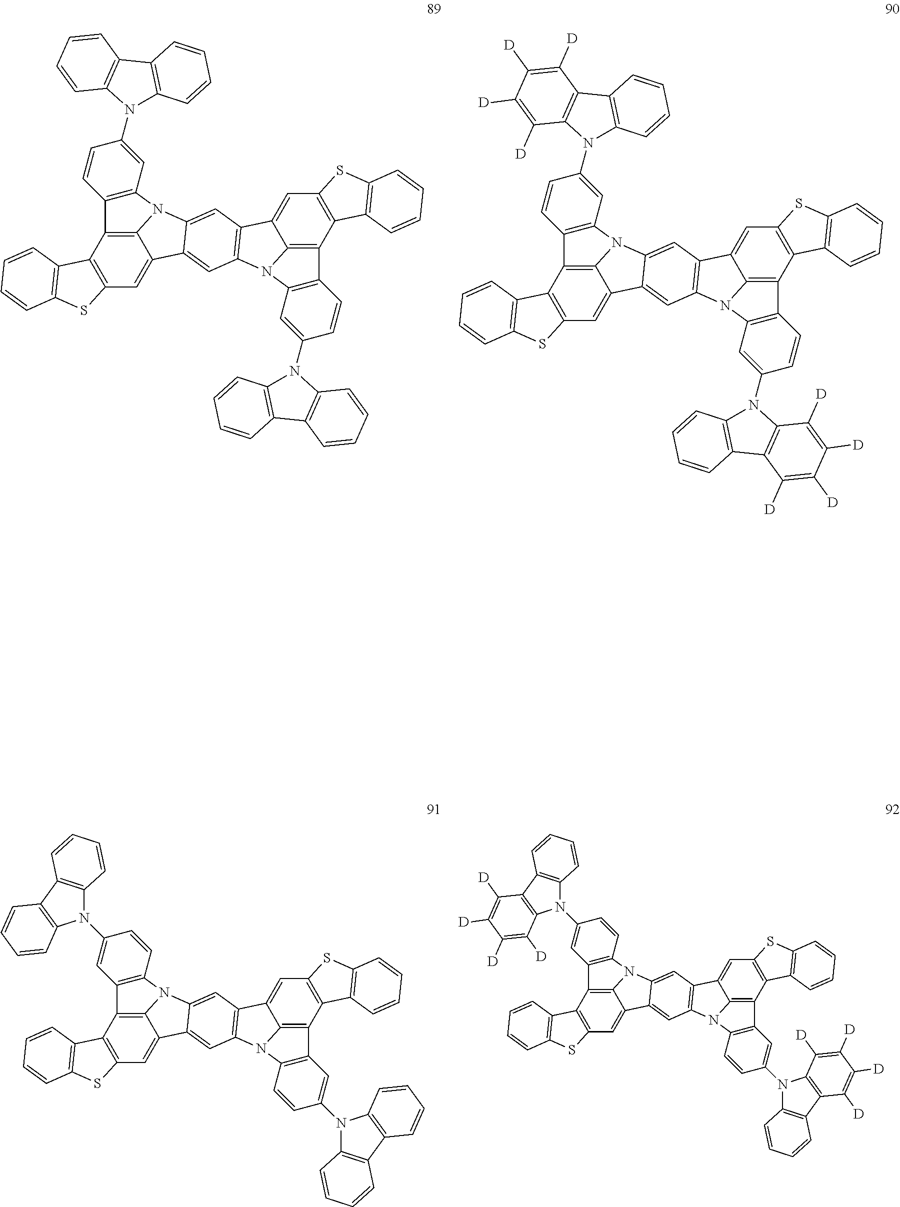

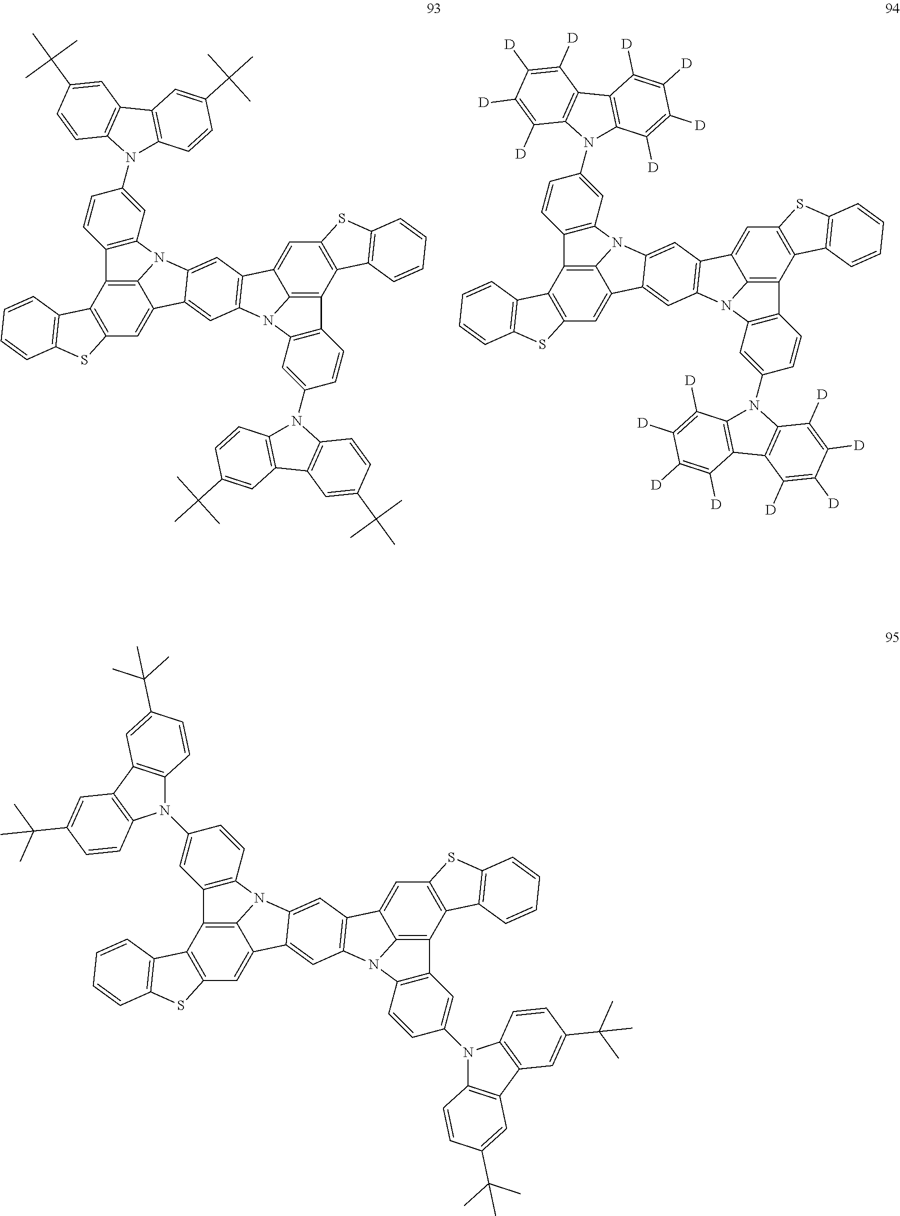

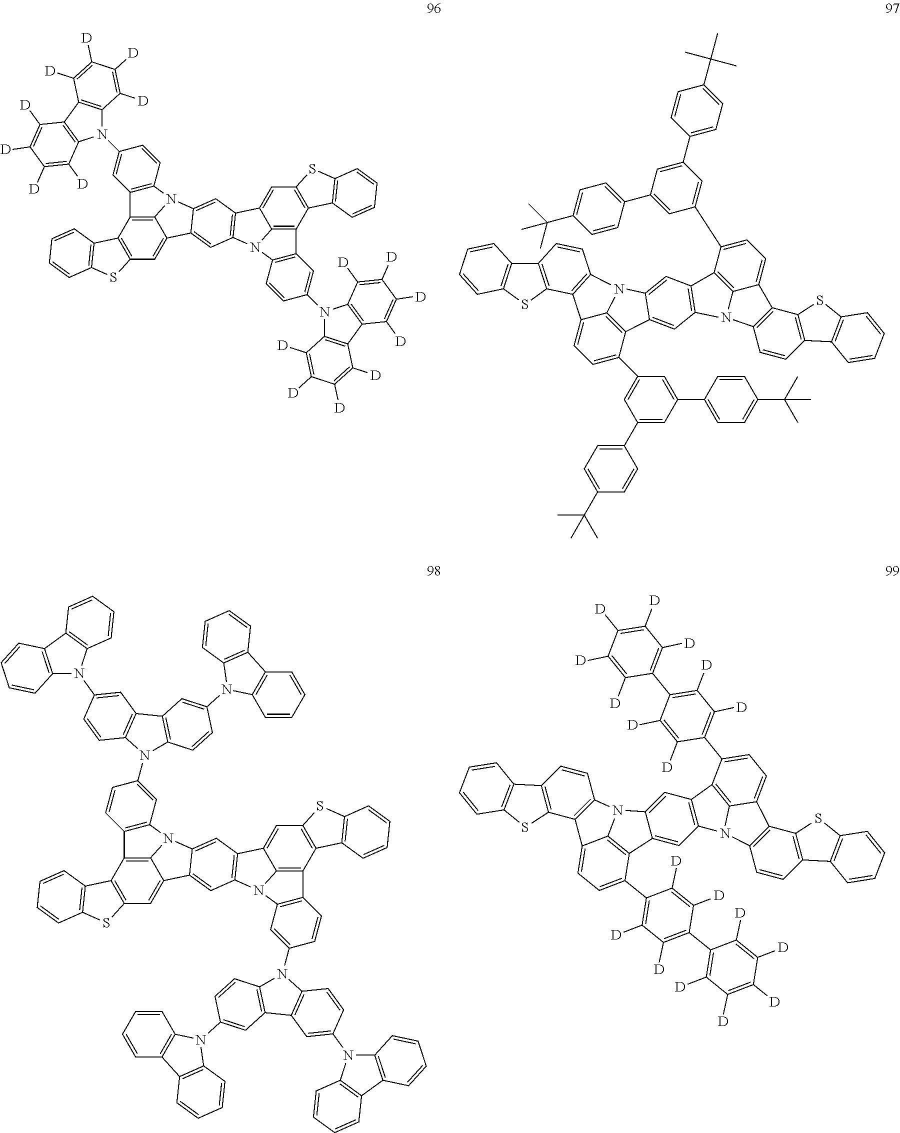

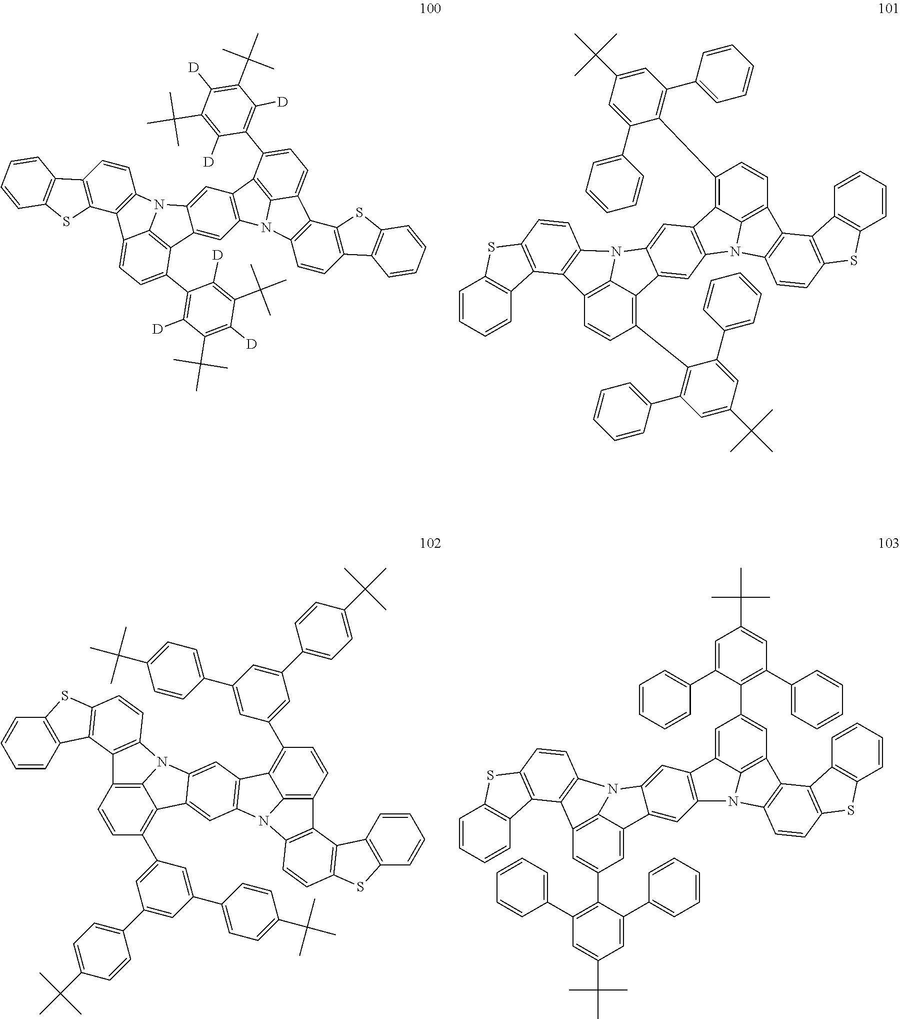

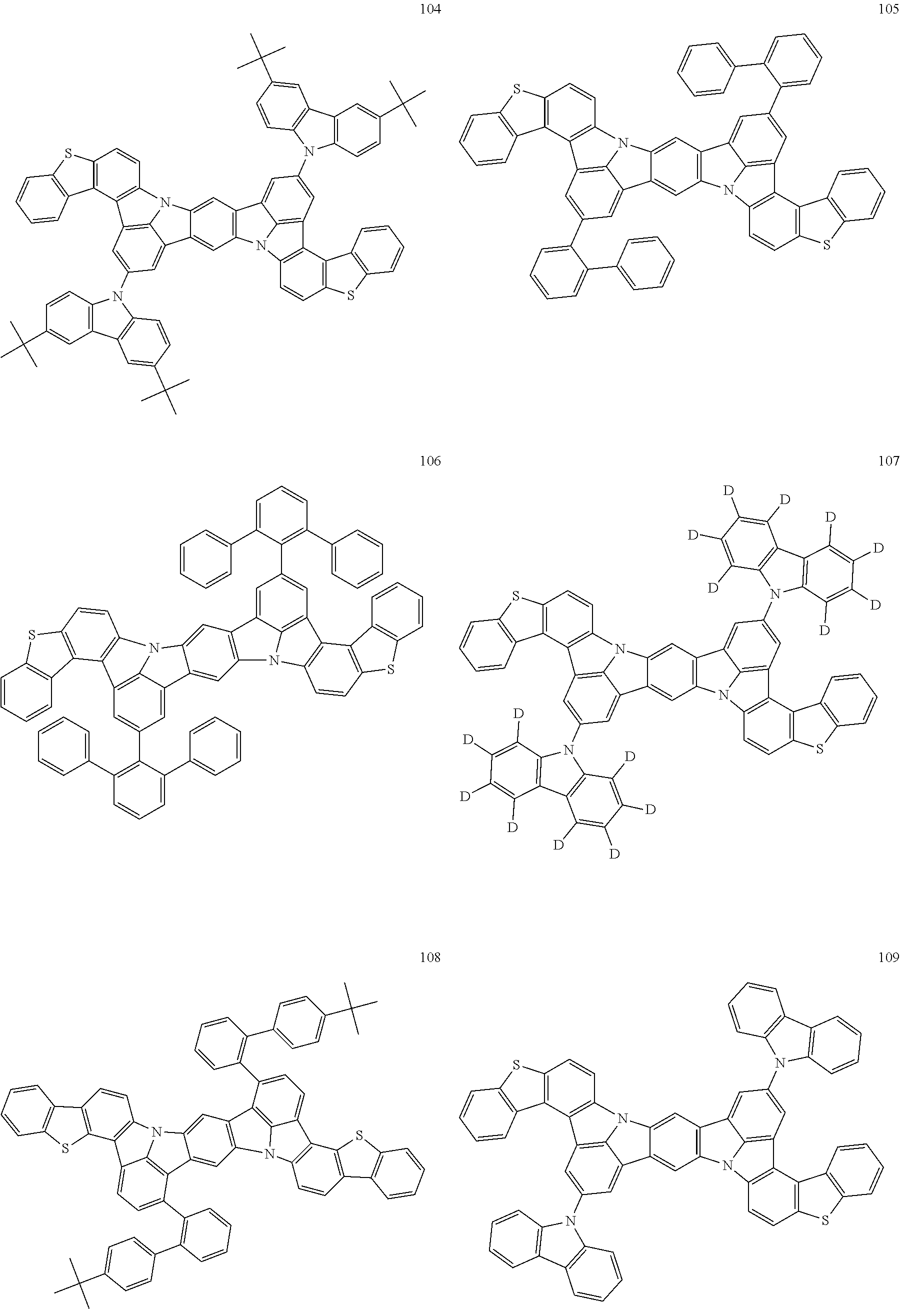

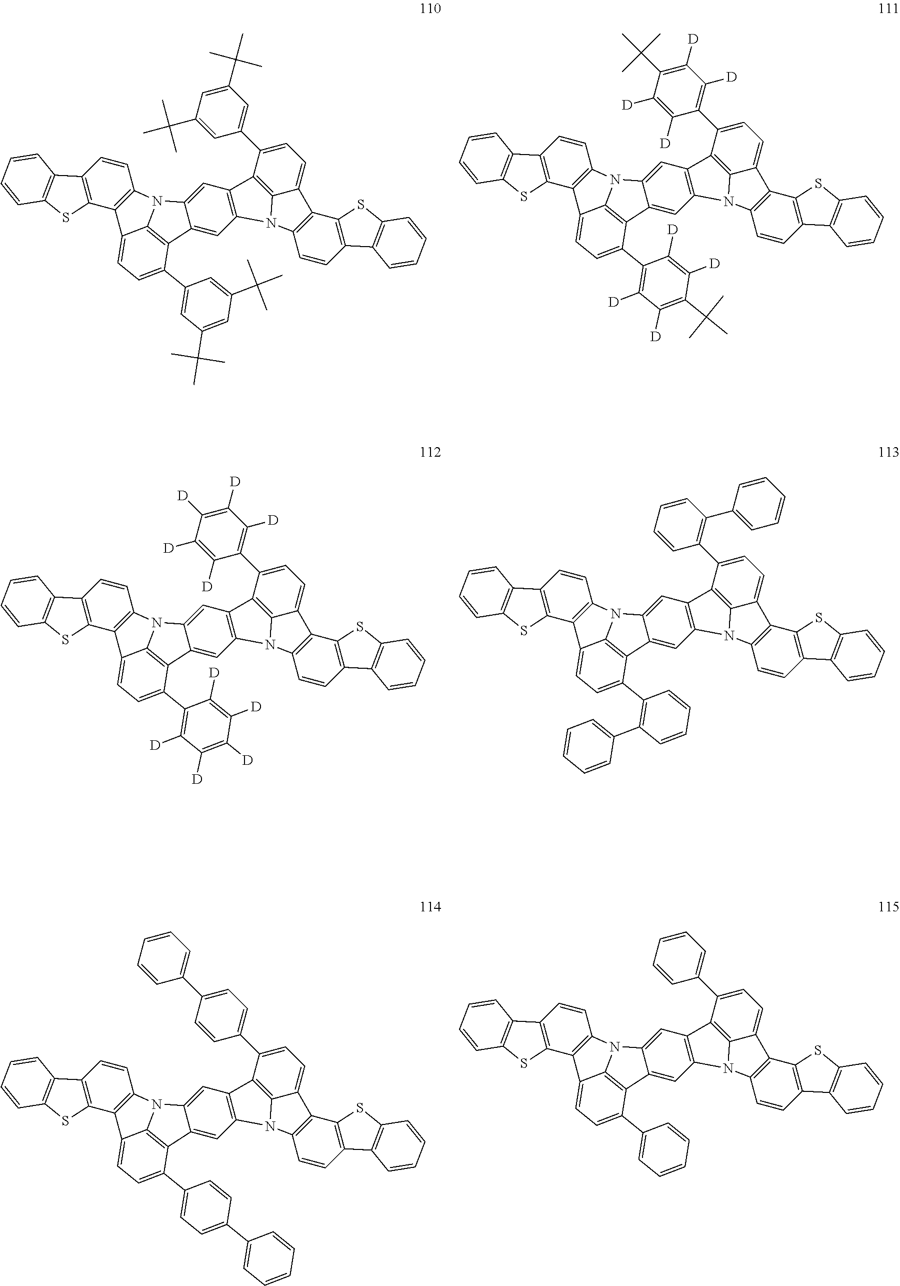

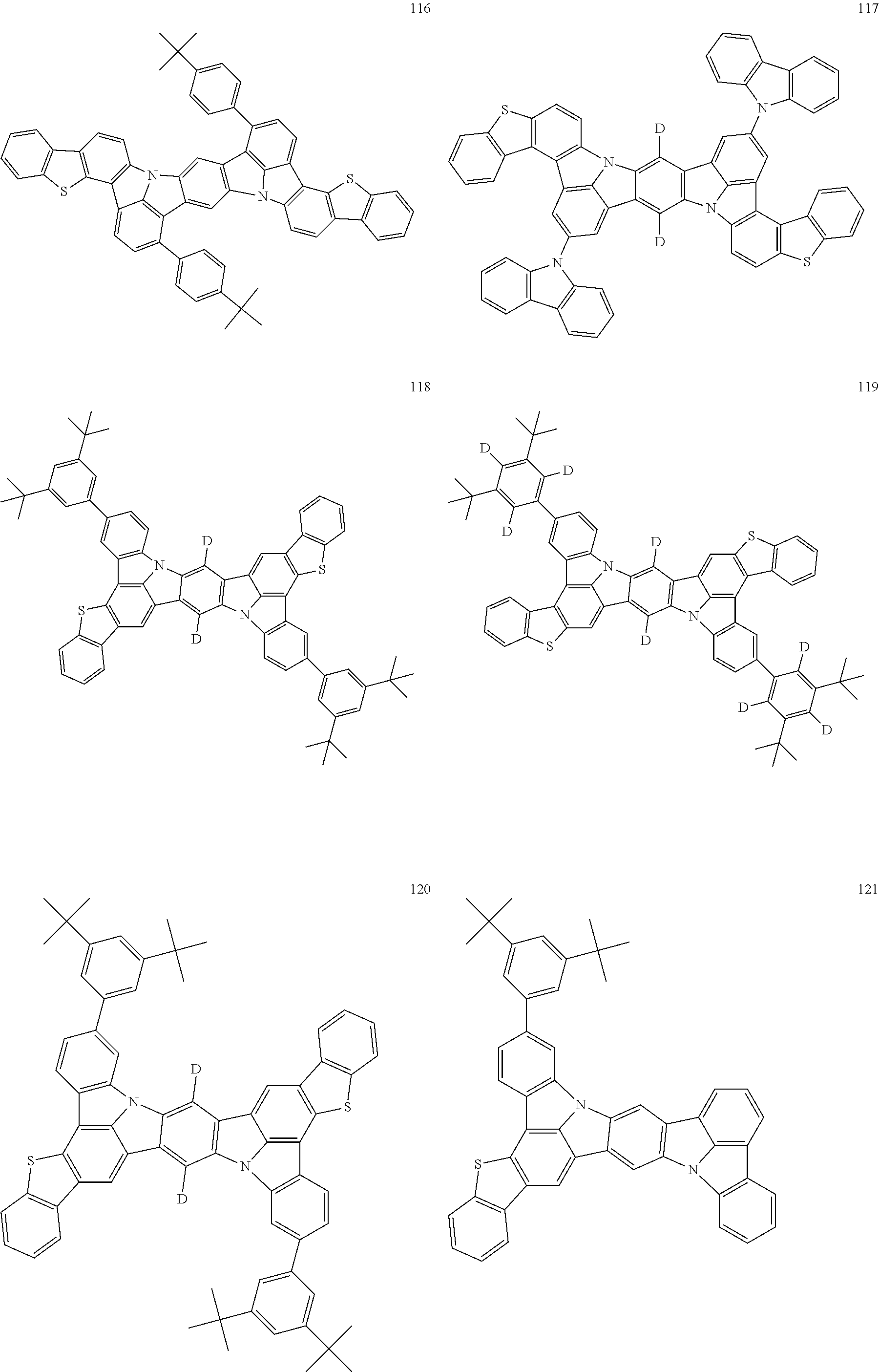

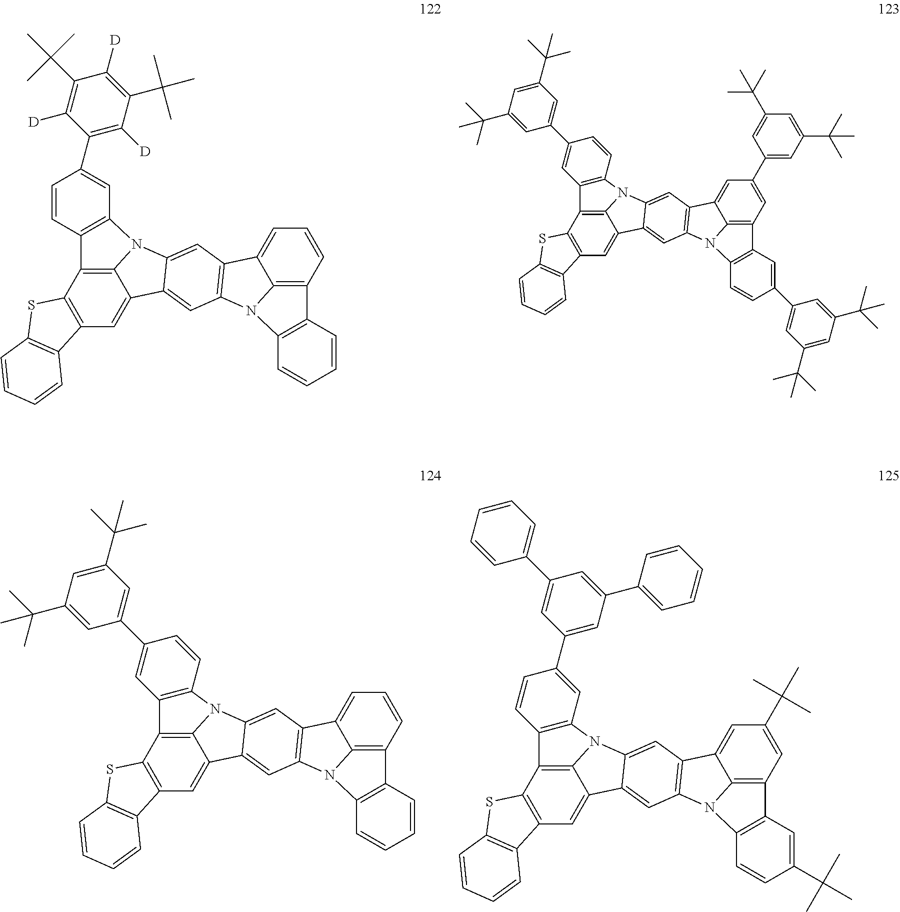 [SMILES: [2H]c1c(-c2ccc3c4c5sc6ccccc6c5cc5c6cc7c(cc6n(c3c2)c54)c2cccc3c4ccccc4n7c32)c([2H])c(C(C)(C)C)c([2H])c1C(C)(C)C ; CC(C)(C)c1cc(-c2ccc3c(c2)c2cc(-c4cc(C(C)(C)C)cc(C(C)(C)C)c4)cc4c5cc6c(cc5n3c24)c2cc3c4ccccc4sc3c3c4cc(-c5cc(C(C)(C)C)cc(C(C)(C)C)c5)ccc4n6c23)cc(C(C)(C)C)c1 ; CC(C)(C)c1cc(-c2ccc3c(c2)c2c4sc5ccccc5c4cc4c5cc6c(cc5n3c42)c2cccc3c4ccccc4n6c32)cc(C(C)(C)C)c1 ; CC(C)(C)c1ccc2c(c1)c1cc(C(C)(C)C)cc3c4cc5c(cc4n2c13)c1cc2c3ccccc3sc2c2c3ccc(-c4cc(-c6ccccc6)cc(-c6ccccc6)c4)cc3n5c12]

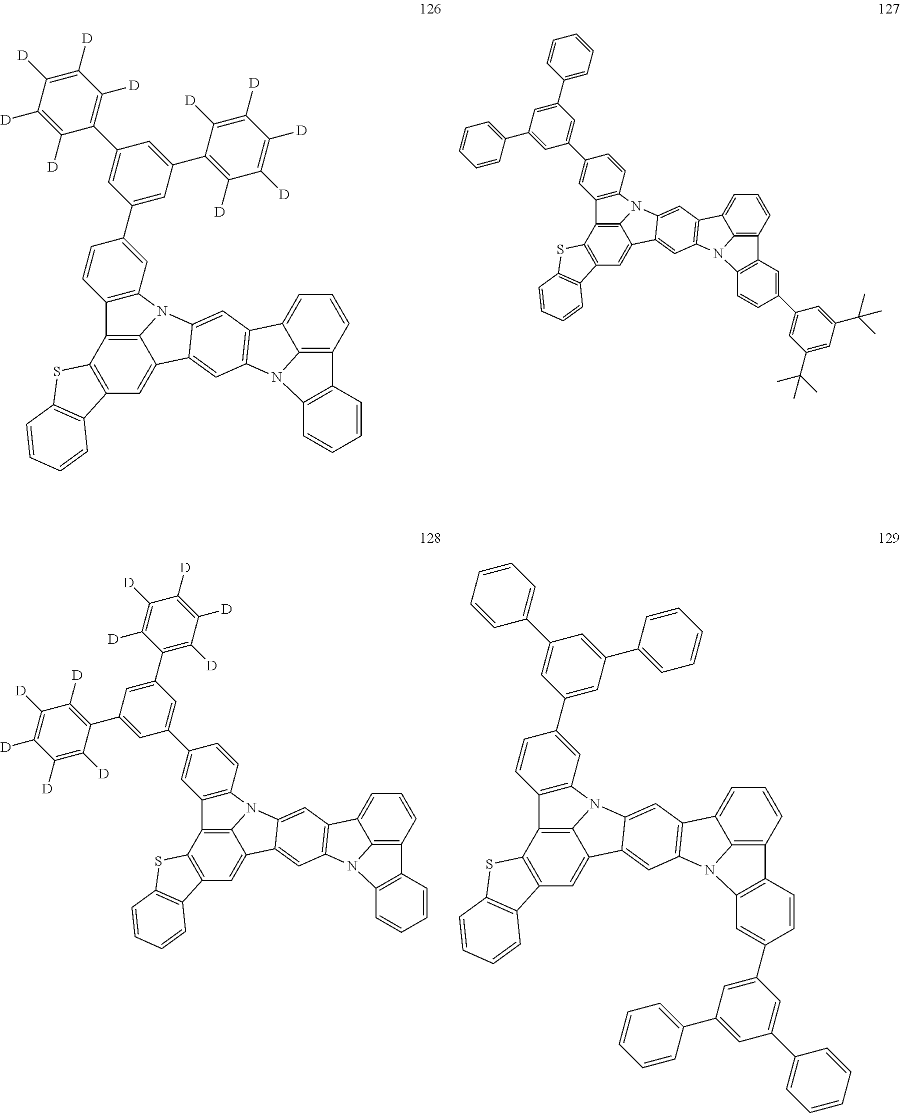

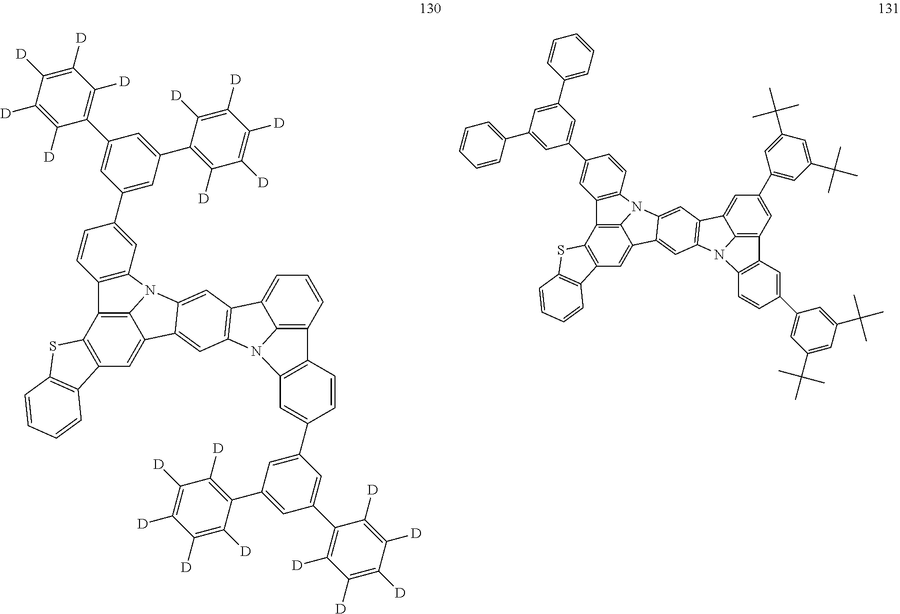 [SMILES: [2H]c1c([2H])c([2H])c(-c2cc(-c3ccc4c5cccc6c7cc8c(cc7n(c4c3)c56)c3cc4c5ccccc5sc4c4c5ccc(-c6cc(-c7c([2H])c([2H])c([2H])c([2H])c7[2H])cc(-c7c([2H])c([2H])c([2H])c([2H])c7[2H])c6)cc5n8c34)cc(-c3c([2H])c([2H])c([2H])c([2H])c3[2H])c2)c([2H])c1[2H] ; CC(C)(C)c1cc(-c2ccc3c(c2)c2cc(-c4cc(C(C)(C)C)cc(C(C)(C)C)c4)cc4c5cc6c(cc5n3c24)c2cc3c4ccccc4sc3c3c4cc(-c5cc(-c7ccccc7)cc(-c7ccccc7)c5)ccc4n6c23)cc(C(C)(C)C)c1]

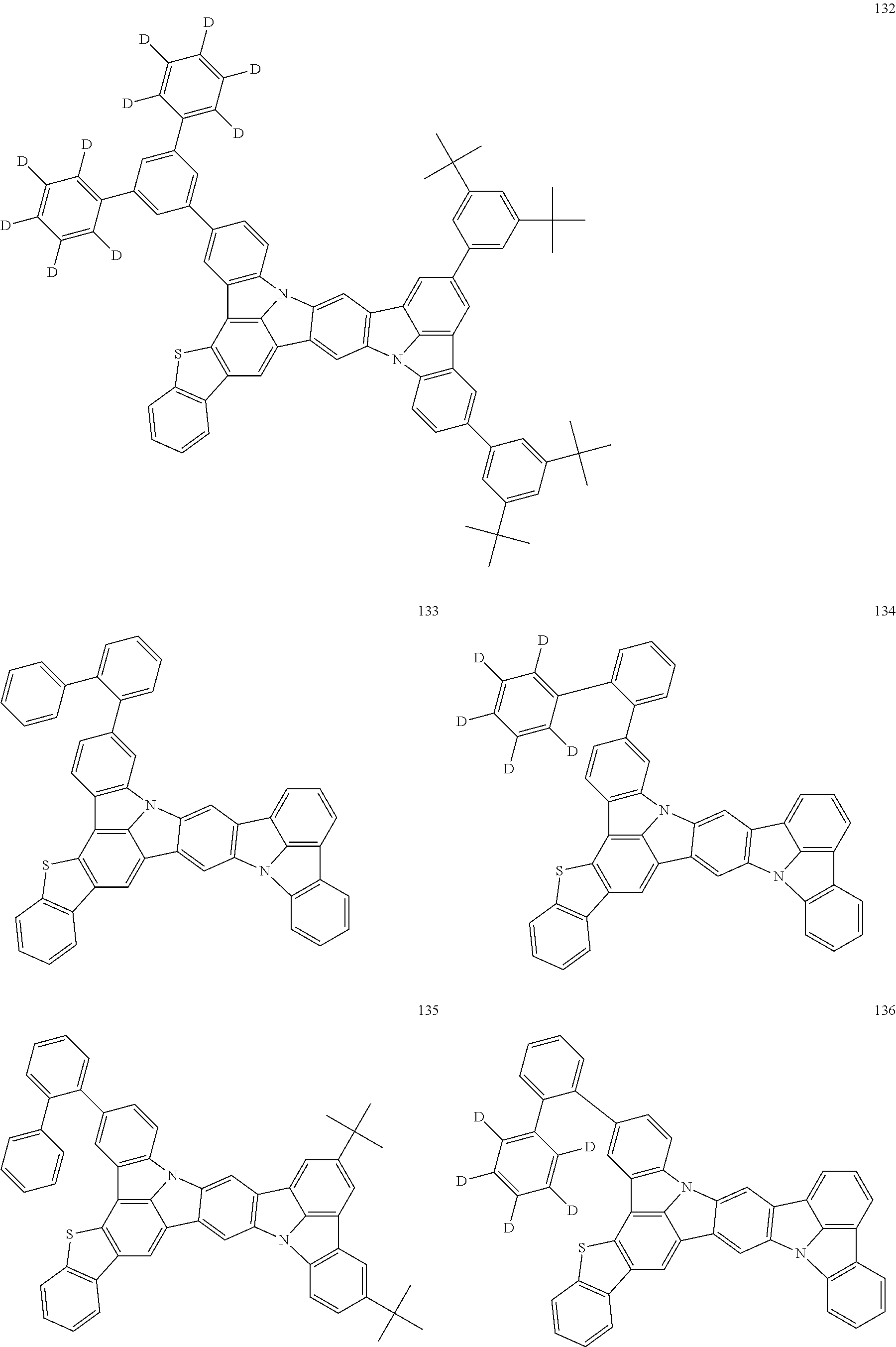

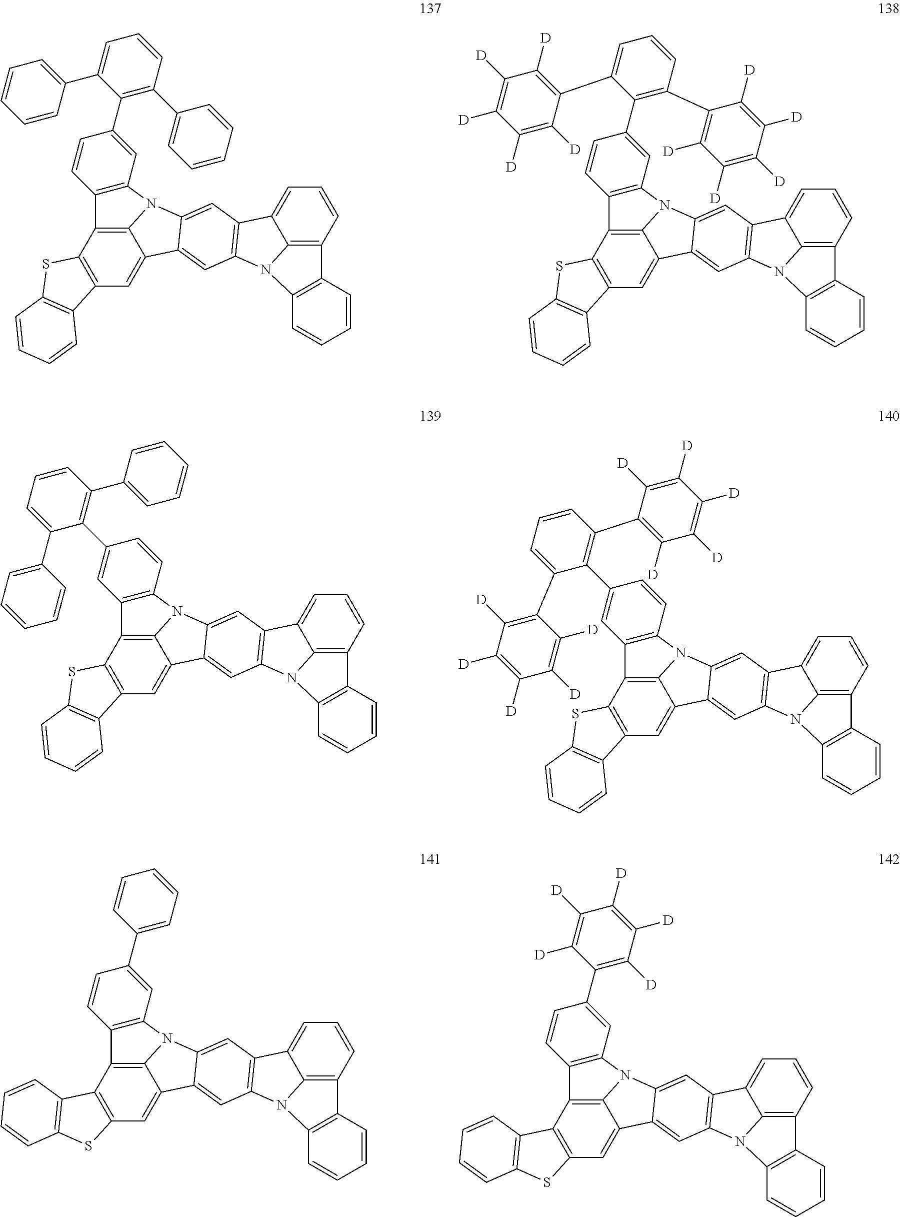

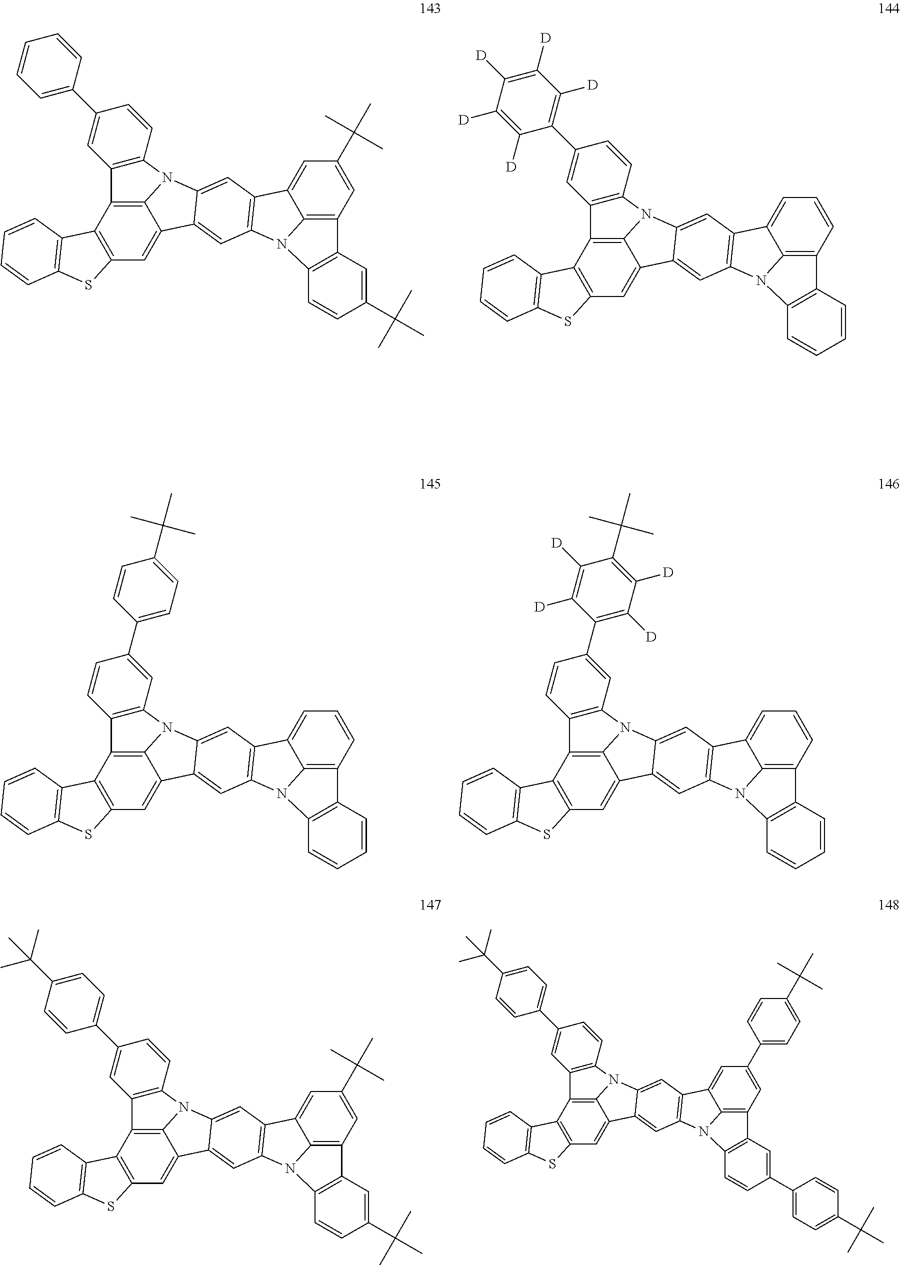

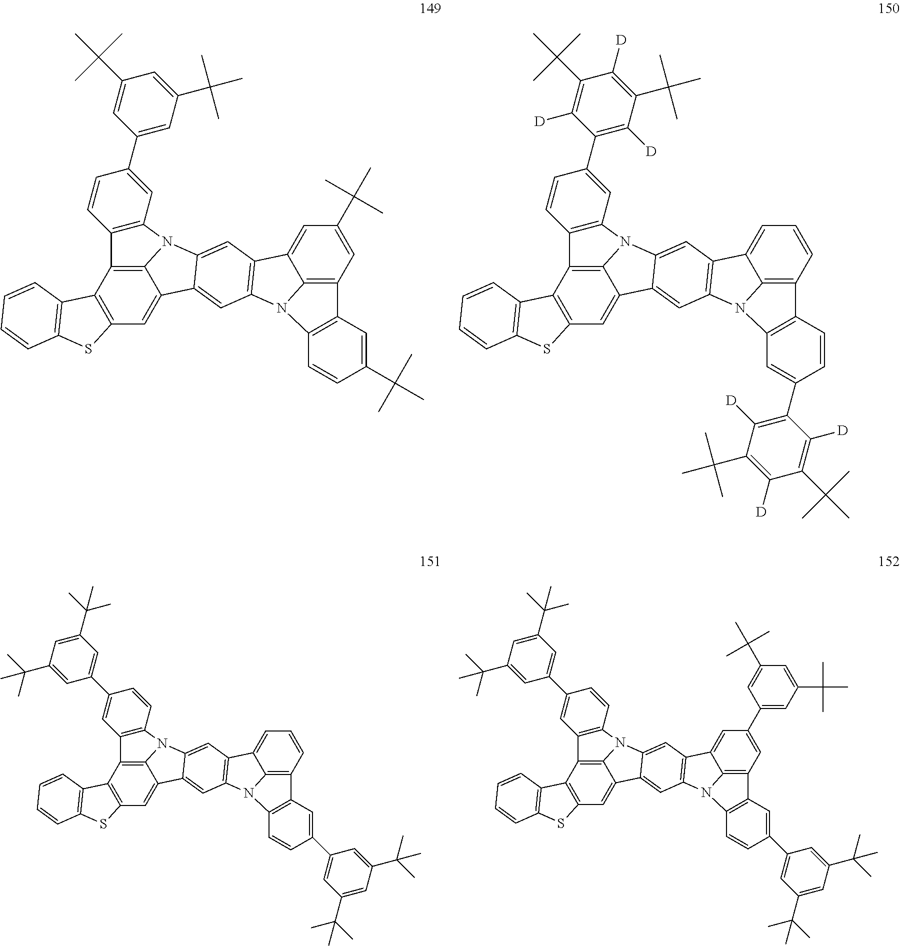

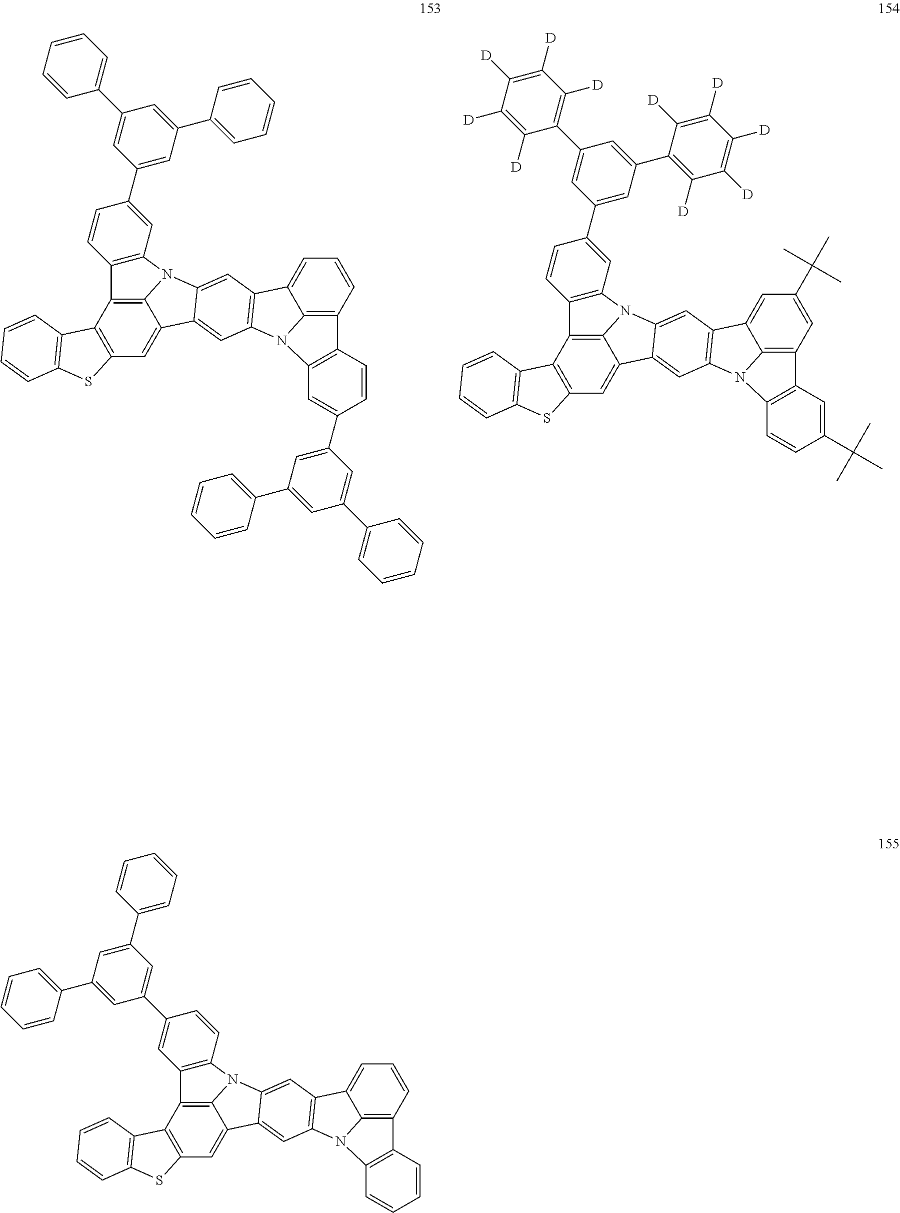 [SMILES: c1ccc(-c2cc(-c3ccccc3)cc(-c3ccc4c5cccc6c7cc8c(cc7n(c4c3)c56)c3cc4sc5ccccc5c4c4c5ccc(-c6cc(-c7ccccc7)cc(-c7ccccc7)c6)cc5n8c34)c2)cc1 ; [2H]c1c([2H])c([2H])c(-c2cc(-c3ccc4c5c6c(cc7c8cc9c(cc8n(c4c3)c75)c3cc(C(C)(C)C)cc4c5cc(C(C)(C)C)ccc5n9c43)sc3ccccc36)cc(-c3c([2H])c([2H])c([2H])c([2H])c3[2H])c2)c([2H])c1[2H] ; c1ccc(-c2cc(-c3ccccc3)cc(-c3ccc4c(c3)c3c5c(cc6c7cc8c(cc7n4c63)c3cccc4c6ccccc6n8c43)sc3ccccc35)c2)cc1]

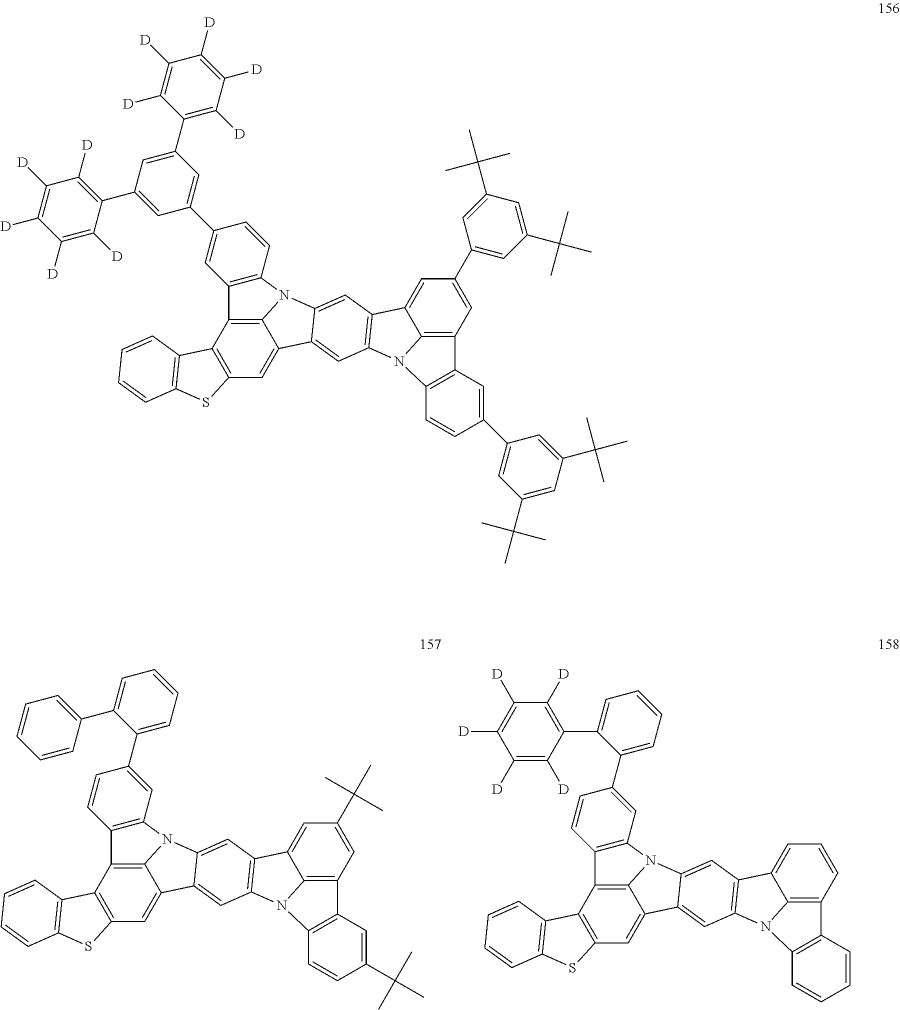 [SMILES: [2H]c1c([2H])c([2H])c(-c2cc(-c3ccc4c(c3)c3c5c(cc6c7cc8c(cc7n4c63)c3cc(-c4cc(C(C)(C)C)cc(C(C)(C)C)c4)cc4c6cc(-c7cc(C(C)(C)C)cc(C(C)(C)C)c7)ccc6n8c43)sc3ccccc35)cc(-c3c([2H])c([2H])c([2H])c([2H])c3[2H])c2)c([2H])c1[2H] ; CC(C)(C)c1ccc2c(c1)c1cc(C(C)(C)C)cc3c4cc5c(cc4n2c13)c1cc2sc3ccccc3c2c2c3ccc(-c4ccccc4-c4ccccc4)cc3n5c12 ; [2H]c1c([2H])c([2H])c(-c2ccccc2-c2ccc3c4c5c(cc6c7cc8c(cc7n(c3c2)c64)c2cccc3c4ccccc4n8c32)sc2ccccc25)c([2H])c1[2H]]

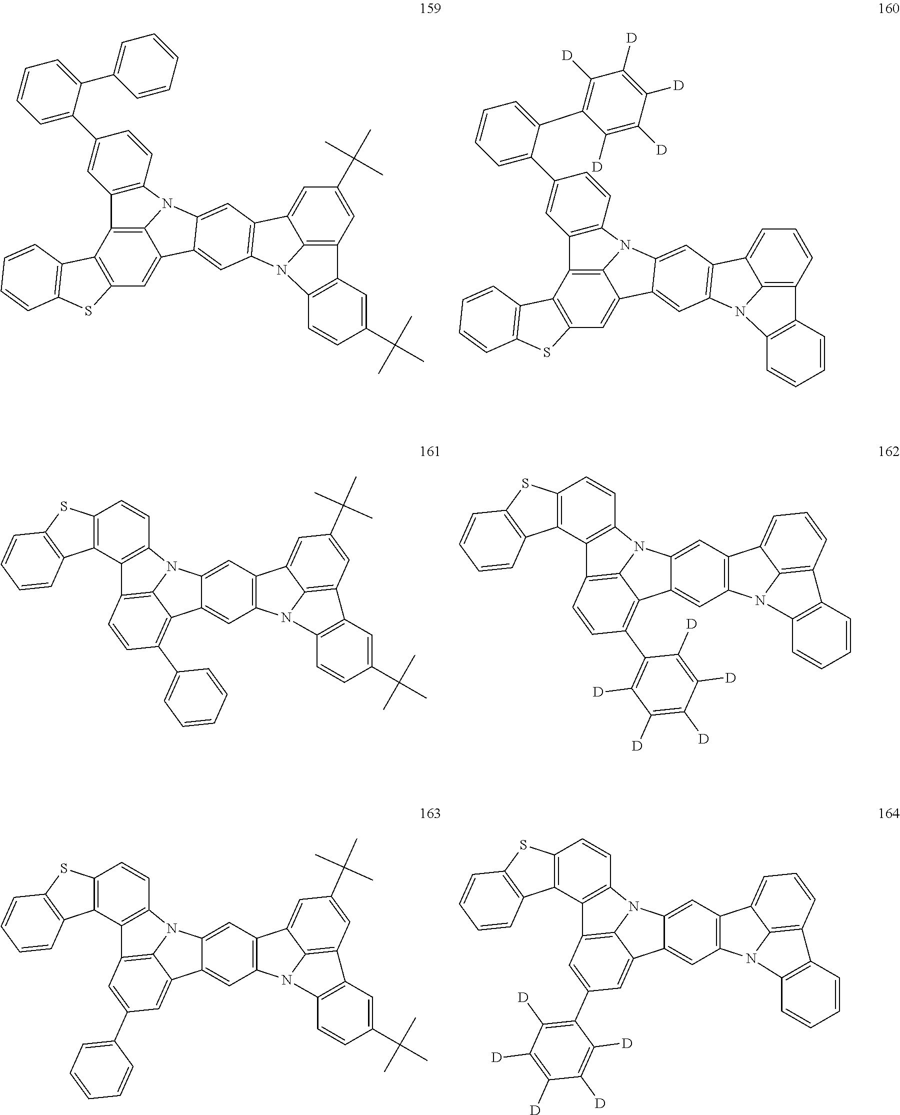

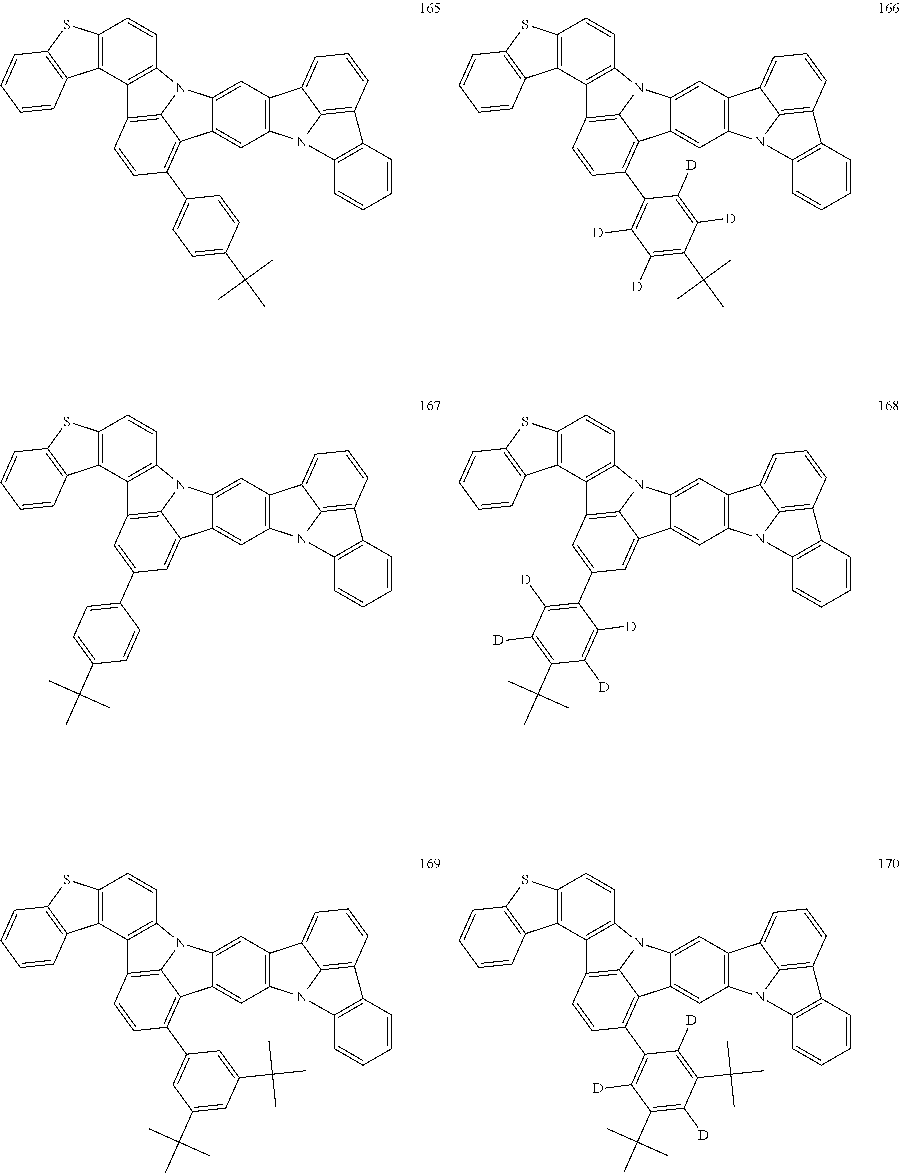

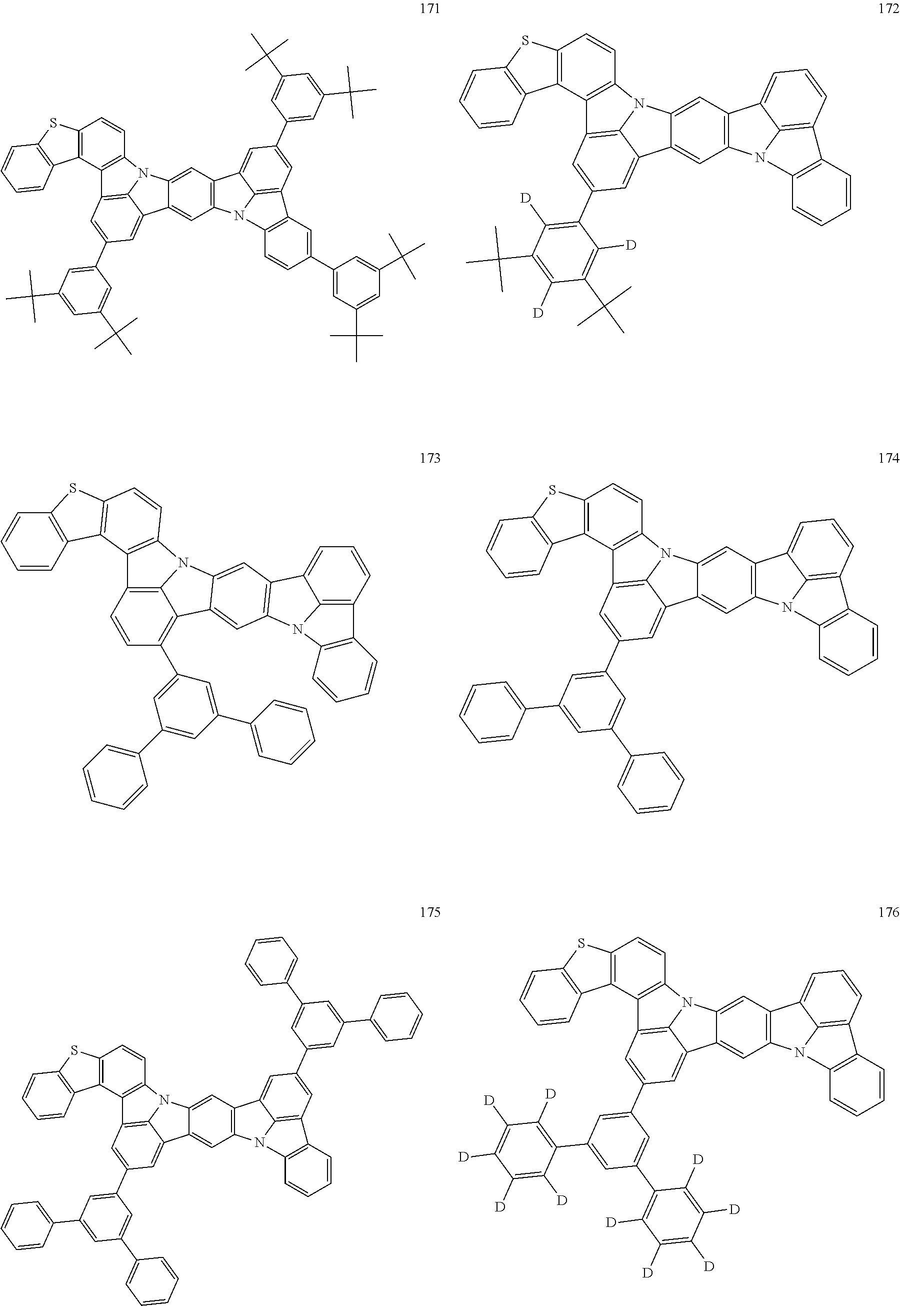

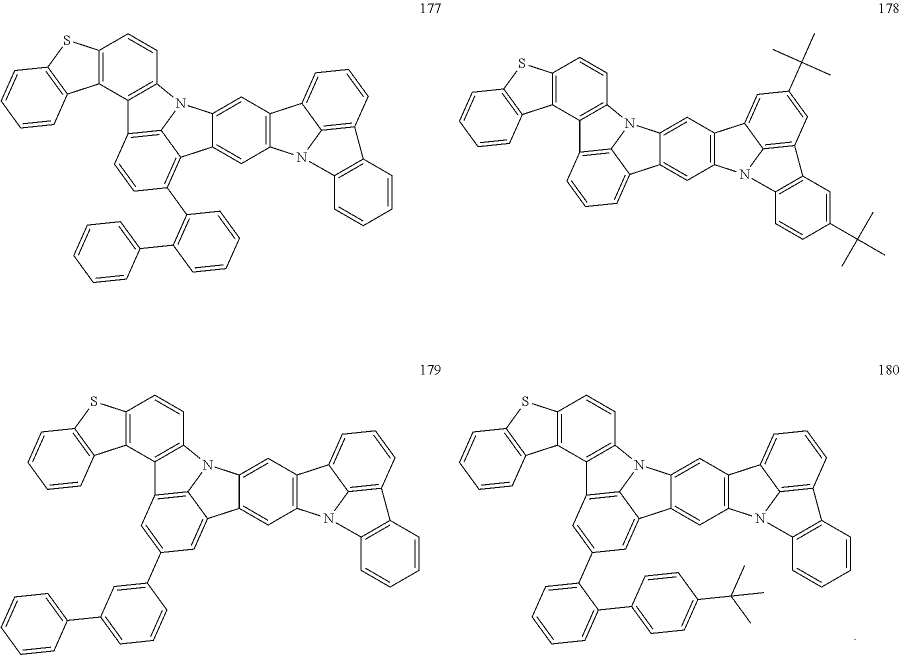 [SMILES: c1ccc(-c2ccccc2-c2ccc3c4c5c(ccc4n4c6cc7c8cccc9c%10ccccc%10n(c7cc6c2c34)c98)sc2ccccc25)cc1 ; CC(C)(C)c1ccc2c(c1)c1cc(C(C)(C)C)cc3c4cc5c(cc4n2c13)c1cccc2c3c4c(ccc3n5c12)sc1ccccc14 ; c1ccc(-c2cccc(-c3cc4c5cc6c(cc5n5c7ccc8sc9ccccc9c8c7c(c3)c45)c3cccc4c5ccccc5n6c43)c2)cc1 ; CC(C)(C)c1ccc(-c2ccccc2-c2cc3c4cc5c(cc4n4c6ccc7sc8ccccc8c7c6c(c2)c34)c2cccc3c4ccccc4n5c32)cc1]

10. The light emitting device of claim 1, wherein the emission layer further comprises a second compound represented by Formula H-1: 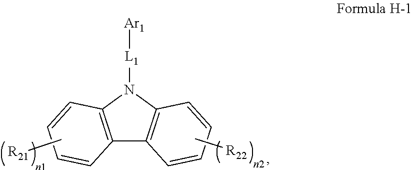
wherein, in Formula H-1,
L₁ is a direct linkage, a substituted or unsubstituted arylene group having 6 to 30 ring-forming carbon atoms, or a substituted or unsubstituted heteroarylene group having 2 to 30 ring-forming carbon atoms,
Ar₁ is a substituted or unsubstituted aryl group having 6 to 30 ring-forming carbon atoms, or a substituted or unsubstituted heteroaryl group having 2 to 30 ring-forming carbon atoms,
R₂₁ and R₂₂ are each independently a hydrogen atom, a deuterium atom, a halogen atom, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, and/or bonded to an adjacent group to form a ring, and
n1 and n2 are each independently an integer of 0 to 4.

11. The light emitting device of claim 1, wherein the emission layer further comprises a third compound represented by Formula H-2: 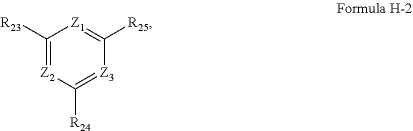
wherein, in Formula H-2,
Z₁ to Z₃ are each independently N or CR₂₆,
at least one from among Z₁ to Z₃ is N, and
R₂₃ to R₂₆ are each independently a hydrogen atom, a deuterium atom, a halogen atom, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, and/or bonded to an adjacent group to form a ring.

12. The light emitting device of claim 1, wherein the emission layer further comprises a fourth compound represented by Formula D-1: 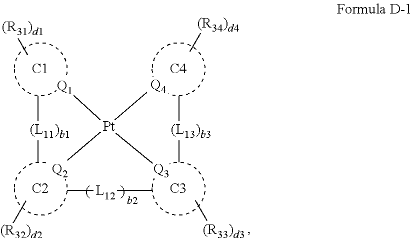
wherein, in Formula D-1,
Q₁ to Q₄ are each independently C or N,
C1 to C4 are each independently a substituted or unsubstituted hydrocarbon ring having 5 to 30 ring-forming carbon atoms, or a substituted or unsubstituted heterocycle having 2 to 30 ring-forming carbon atoms,
L₁₁ to L₁₃ are each independently a direct linkage, 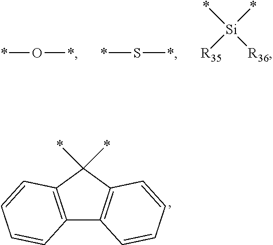
a substituted or unsubstituted divalent alkyl group having 1 to 20 carbon atoms, a substituted or unsubstituted arylene group having 6 to 30 ring-forming carbon atoms, or a substituted or unsubstituted heteroarylene group having 2 to 30 ring-forming carbon atoms,
b1 to b3 are each independently 0 or 1,
R₃₁ to R₃₆ are each independently a hydrogen atom, a deuterium atom, a halogen atom, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, and/or bonded to an adjacent group to form a ring, and
d1 to d4 are each independently an integer of 0 to 4.

13. A fused polycyclic compound represented by Formula 1: 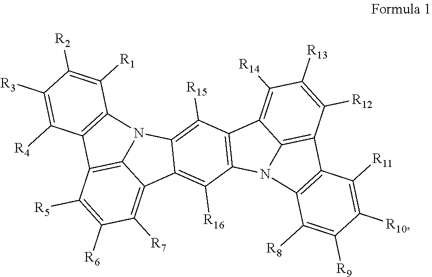
wherein, in Formula 1,
R₁ to R₁₆ are each independently a hydrogen atom, a deuterium atom, a halogen atom, a cyano group, a substituted or unsubstituted silyl group, a substituted or unsubstituted amine group, a substituted or unsubstituted oxy group, a substituted or unsubstituted thio group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and/or bonded to an adjacent group to form a ring,
at least one pair of adjacent groups selected from among R₁ to R₁₄ are bonded to each other to form a substituent represented by Formula 2: Formula 2 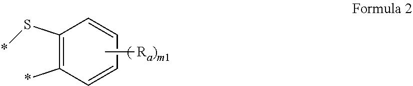
wherein, in Formula 2,
—* is a bonding site to a core of Formula 1,
Rₐ is a hydrogen atom, a deuterium atom, a halogen atom, a cyano group, a substituted or unsubstituted silyl group, a substituted or unsubstituted amine group, a substituted or unsubstituted oxy group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and/or bonded to an adjacent group to form a ring, and
m1 is an integer of 0 to 4.

14. The fused polycyclic compound of claim 13, wherein the fused polycyclic compound represented by Formula 1 is represented by Formula 3-1 or Formula 3-2: 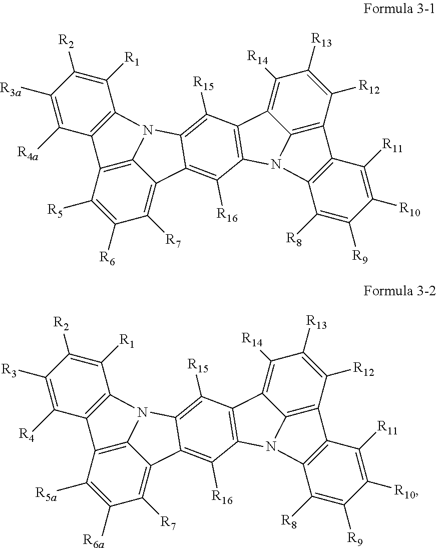
wherein, in Formula 3-1,
R₃ₐ and R₄ₐ are bonded to each other to form the substituent represented by Formula 2, and
wherein, in Formula 3-2,
R₅ₐ and R₄ₐ are bonded to each other to form the substituent represented by Formula 2, and
R₁ to R₁₆ are the same as defined in Formula 1.

15. The fused polycyclic compound of claim 13, wherein the fused polycyclic compound represented by Formula 1 is represented by Formula 4-1 or Formula 4-2: 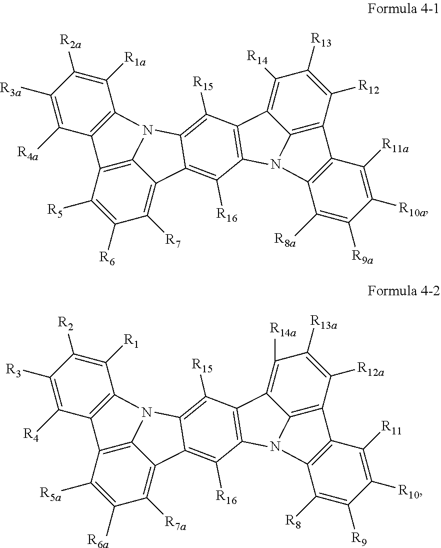
wherein, in Formula 4-1 and Formula 4-2,
R₁ₐ to R₁₄ₐ are each independently a hydrogen atom, a deuterium atom, a halogen atom, a cyano group, a substituted or unsubstituted silyl group, a substituted or unsubstituted amine group, a substituted or unsubstituted oxy group, a substituted or unsubstituted thio group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and/or bonded to an adjacent group to form a ring,
wherein, in Formula 4-1,
one pair of adjacent groups selected from among R₁ₐ to R₄ₐ are bonded to each other to form the substituent represented by Formula 2, and one pair of adjacent groups selected from among R₈ₐ to R₁₁ₐ are bonded to each other to form the substituent represented by Formula 2, and
wherein, in Formula 4-2,
one pair of adjacent groups selected from among R₅ₐ to R₇ₐ are bonded to each other to form the substituent represented by Formula 2, and one pair of adjacent groups selected from among R₁2a to R₁4R are bonded to each other to form the substituent represented by Formula 2, and
R₁ to R₁₆ are the same as defined in Formula 1.

16. The fused polycyclic compound of claim 13, wherein the fused polycyclic compound represented by Formula 1 is represented by Formula 5-1 or Formula 5-2: 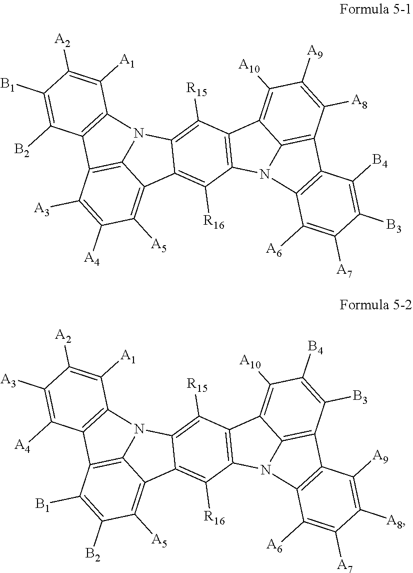
wherein, in Formula 5-1 and Formula 5-2,
A₁ to A₁₀ are each independently a hydrogen atom, a deuterium atom, a halogen atom, a cyano group, a substituted or unsubstituted silyl group, a substituted or unsubstituted amine group, a substituted or unsubstituted oxy group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and/or bonded to an adjacent group to form a ring,
B₁ and B₂ are bonded to each other to form the substituent represented by Formula 2, and B₃ and B₄ are bonded to each other to form the substituent represented by Formula 2, and
R₁₅ to R₁₆ are the same as defined in Formula 1.

17. The fused polycyclic compound of claim 16, wherein the fused polycyclic compound represented by Formula 1 is represented by any one from among Formula 6-1 to Formula 6-4: 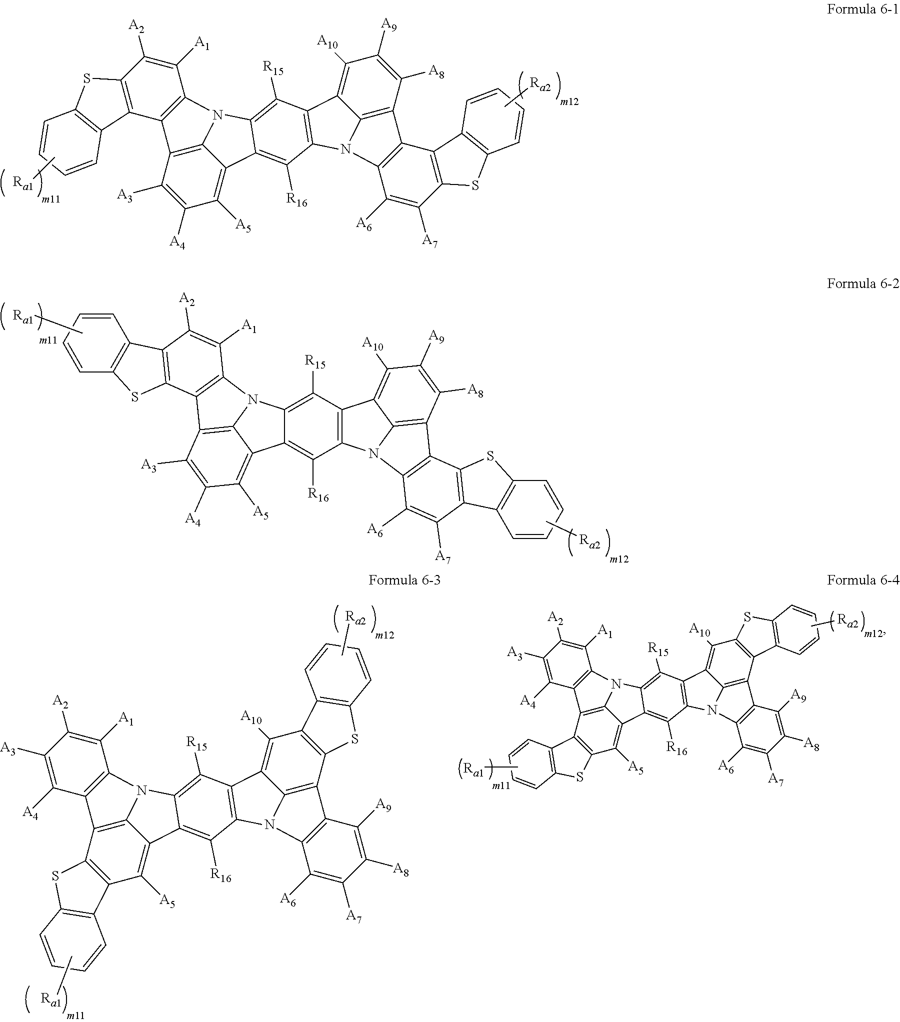
wherein, in Formula 6-1 to Formula 6-4,
Rₐ₁ and Rₐ2 are each independently a hydrogen atom, a deuterium atom, a halogen atom, a cyano group, a substituted or unsubstituted silyl group, a substituted or unsubstituted amine group, a substituted or unsubstituted oxy group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and/or bonded to an adjacent group to form a ring,
m11 and m12 are each independently an integer of 0 to 4, and
R₁₅, R₁₆, and A₁ to A₁₀ are the same as respectively defined in Formula 1, Formula 5-1, and Formula 5-2.

18. The fused polycyclic compound of claim 16, wherein the fused polycyclic compound represented by Formula 1 is represented by any one from among Formula 7-1 to Formula 7-4: 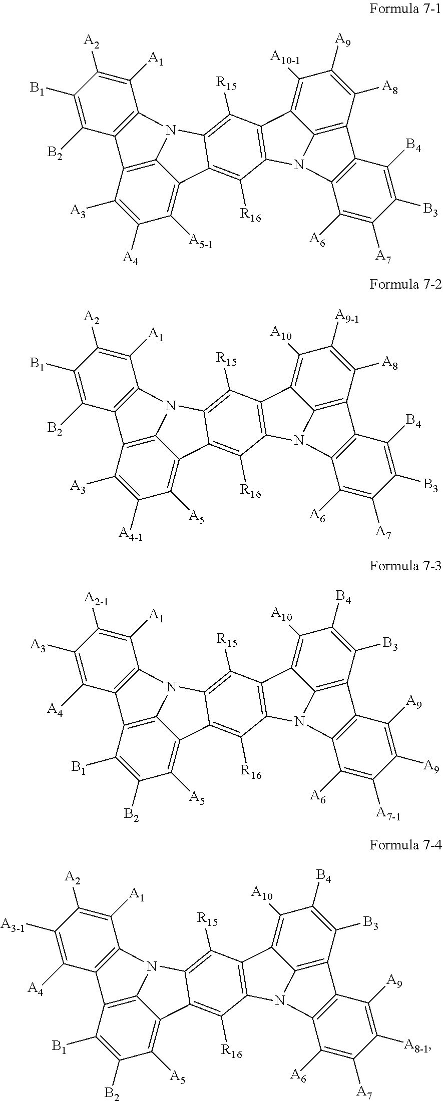
wherein, in Formula 7-1 to Formula 7-4,
A₂₋₁ to A₅₋₁ and A₇₋₁ to A₁₀₋₁ are each independently a substituted or unsubstituted aryl group having 6 to 30 ring-forming carbon atoms, or a substituted or unsubstituted heteroaryl group having 2 to 30 ring-forming carbon atoms, and
R₁₅, R₁₆, A₁ to A₁₀, and B₁ to B₄ are the same as respectively defined in Formula 1, Formula 5-1, and Formula 5-2.

19. The fused polycyclic compound of claim 18, wherein A₂₋₁ to A₅₋₁ and A₇₋₁ to A₁₀₋₁ are each independently represented by any one from among Formula 8-1 to Formula 8-4: 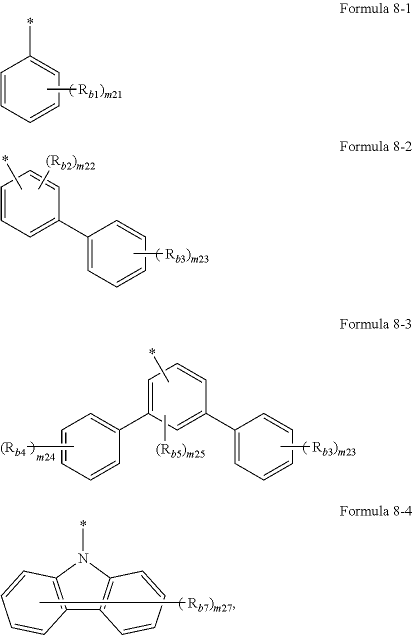
wherein, in Formula 8-1 to Formula 8-4,
R_{b1} to R_{b7} are each independently a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
m21, m23, m24, and m26 are each independently an integer of 0 to 5,
m22 is an integer of 0 to 4,
m25 is an integer of 0 to 3, and
m27 is an integer of 0 to 8.

20. The fused polycyclic compound of claim 13, wherein the fused polycyclic compound represented by Formula 1 comprises at least one from among compounds represented by Compound Group 1: 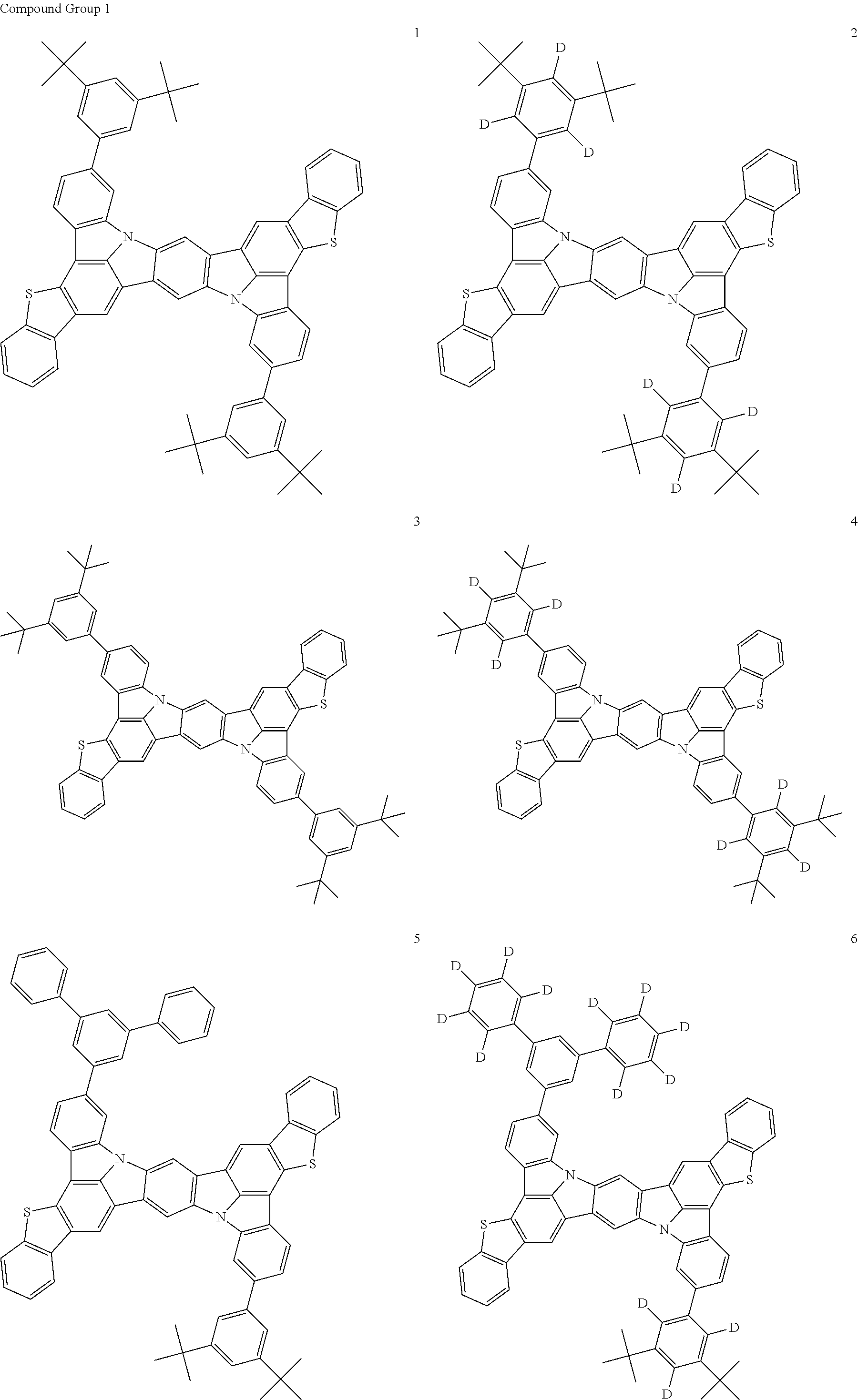 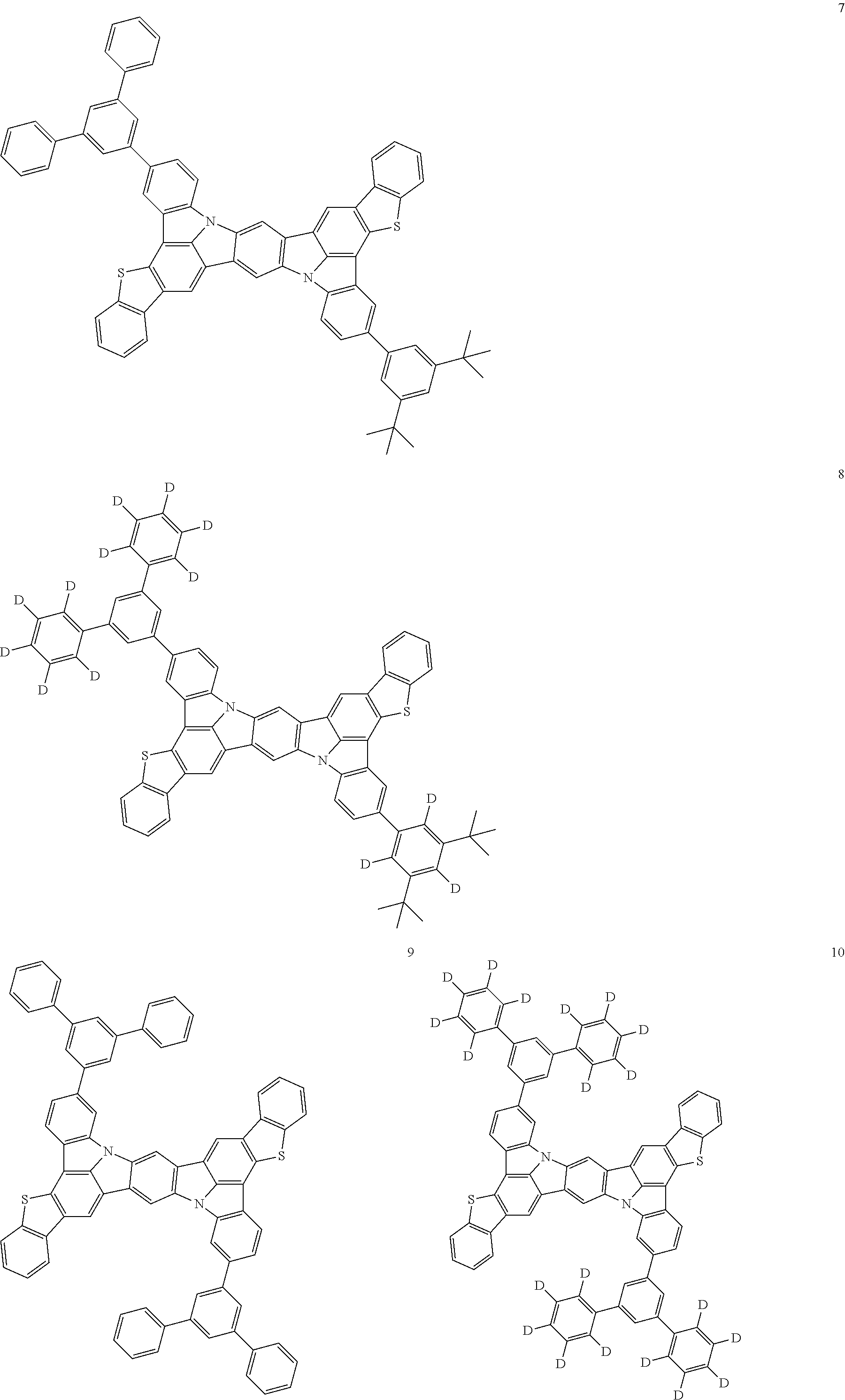 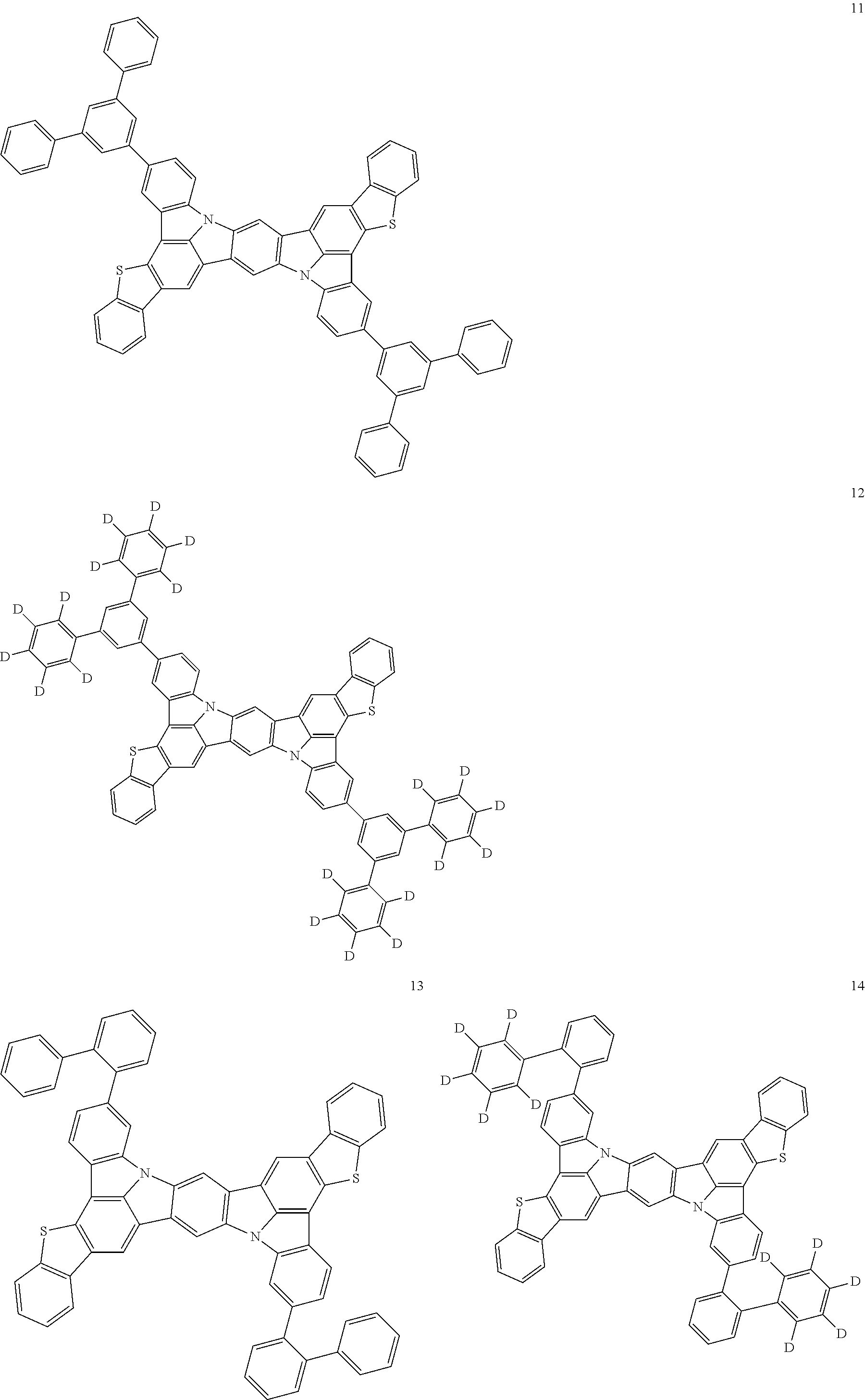 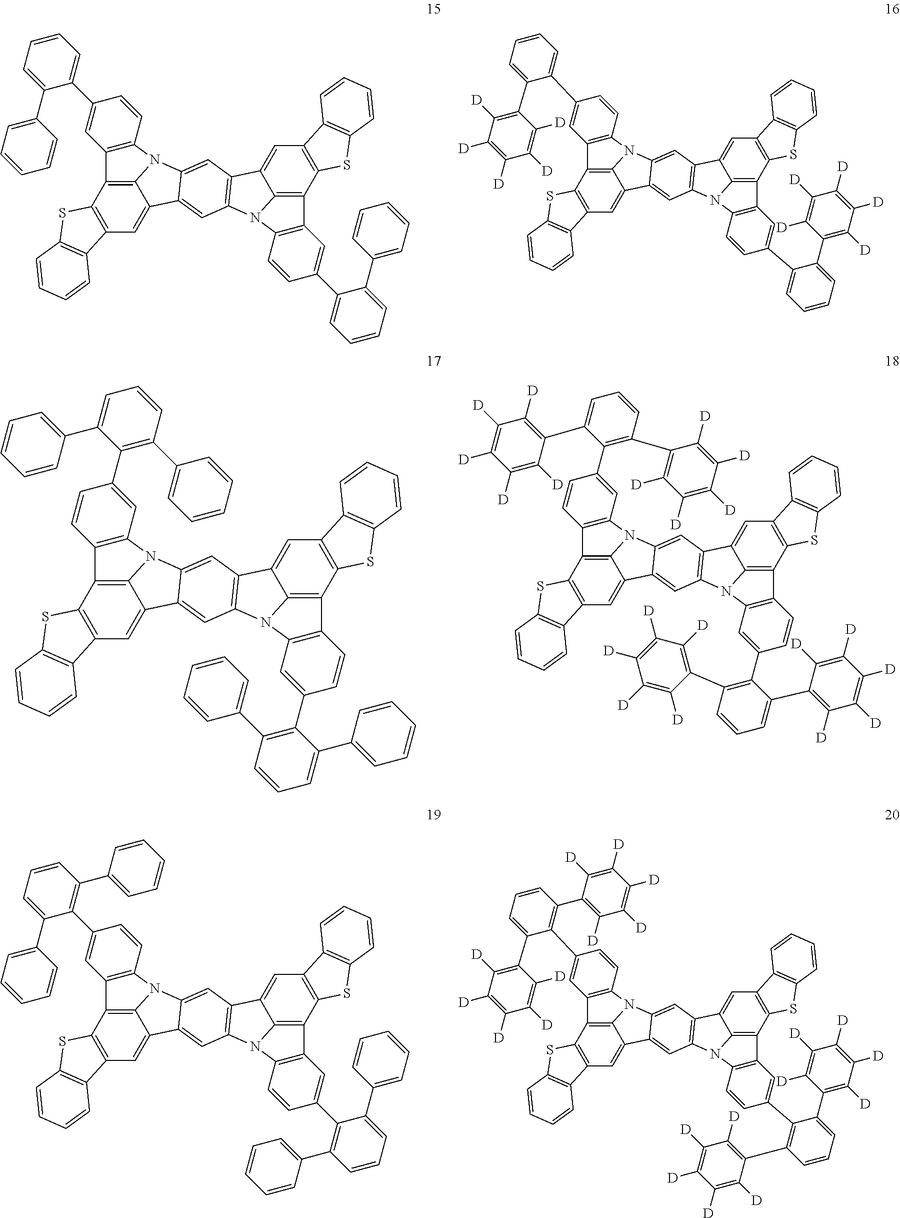 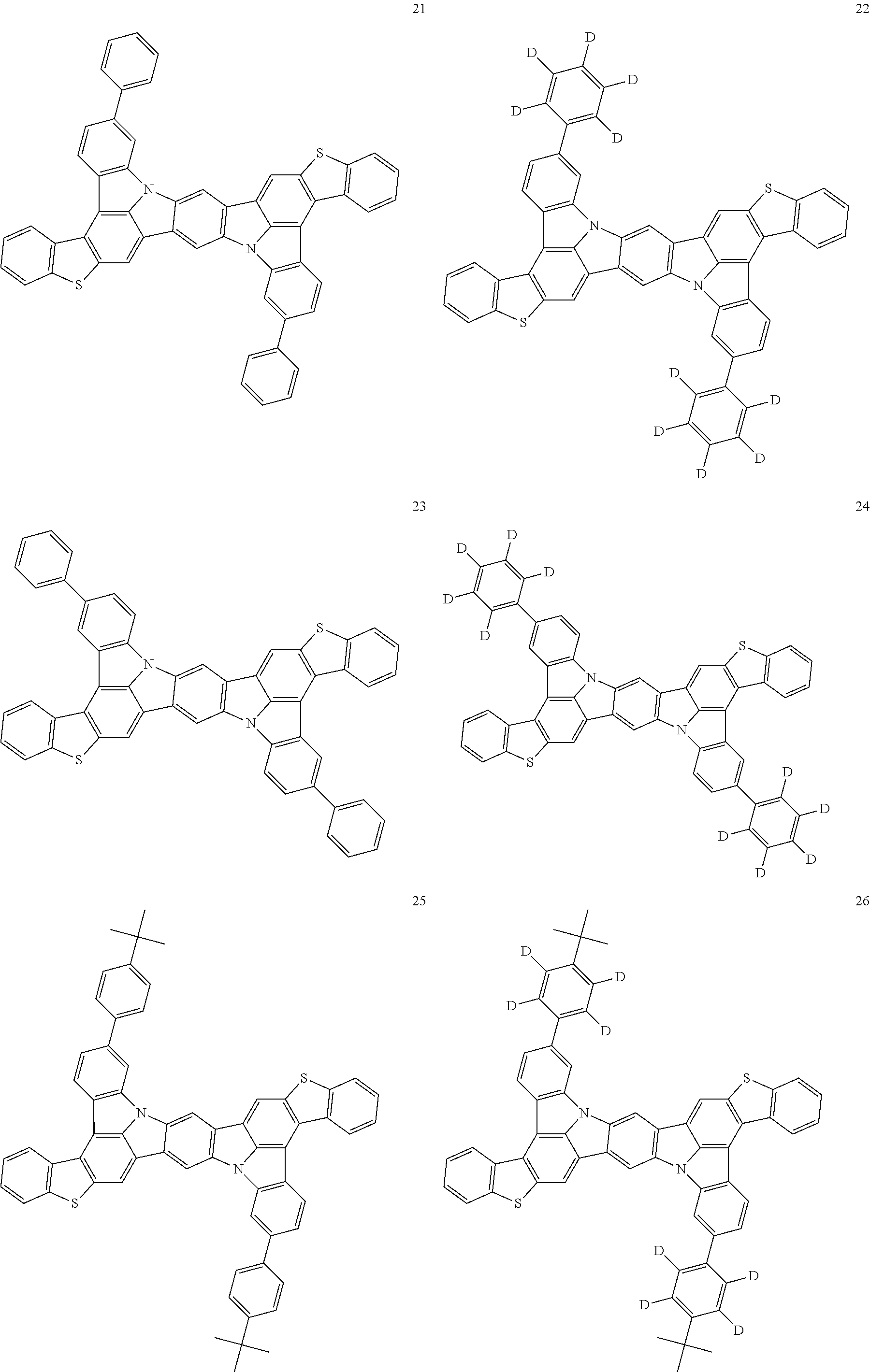 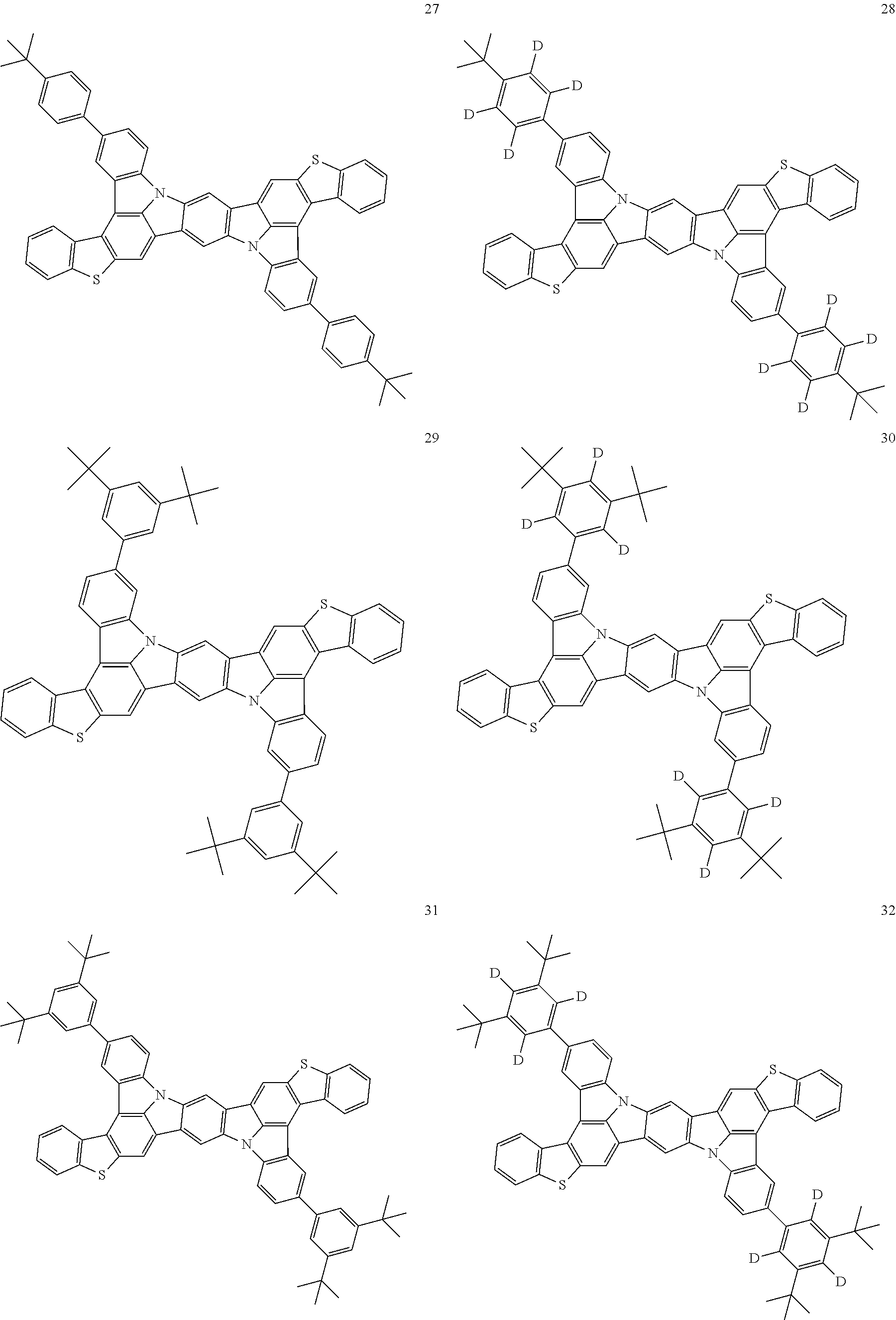 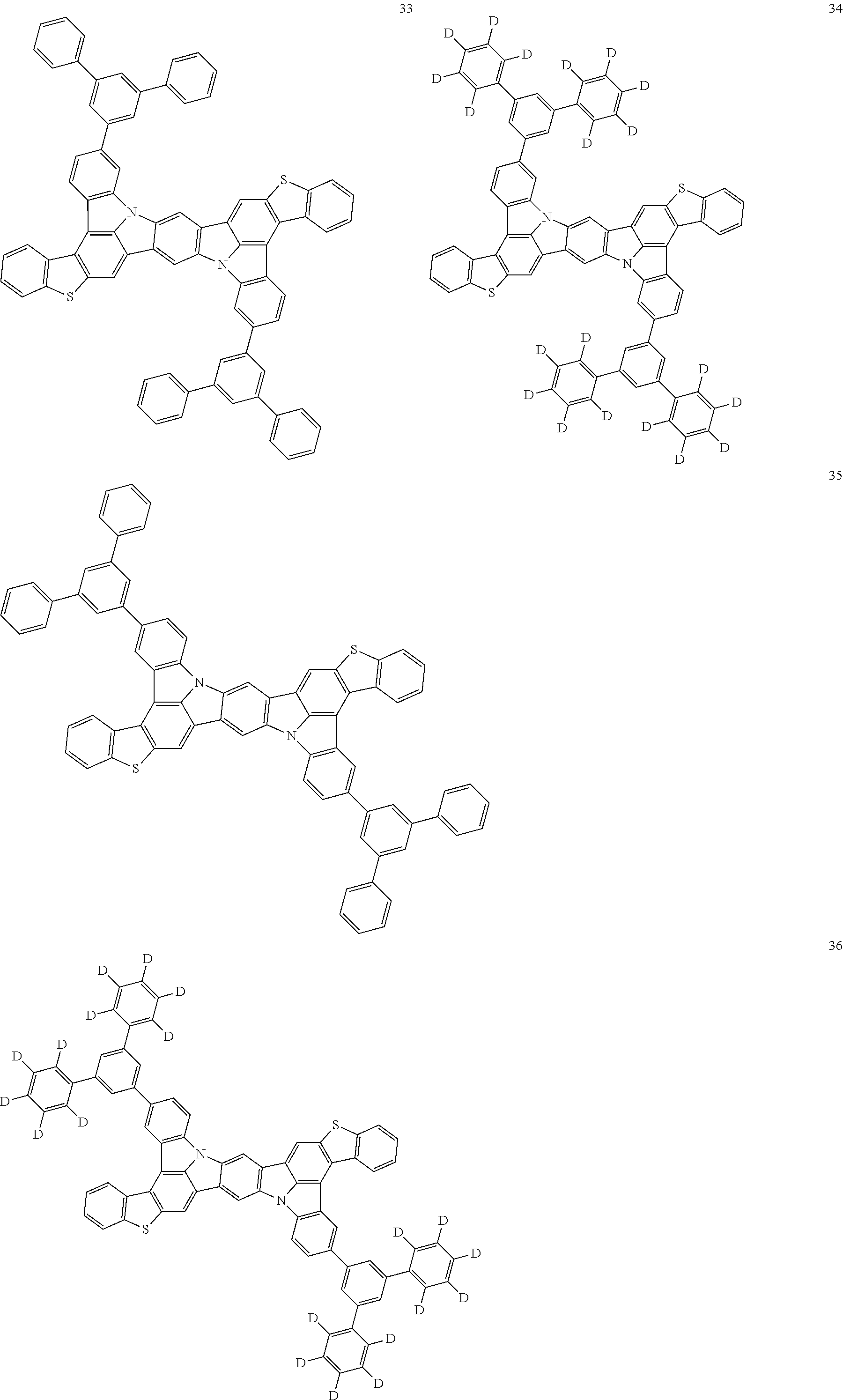 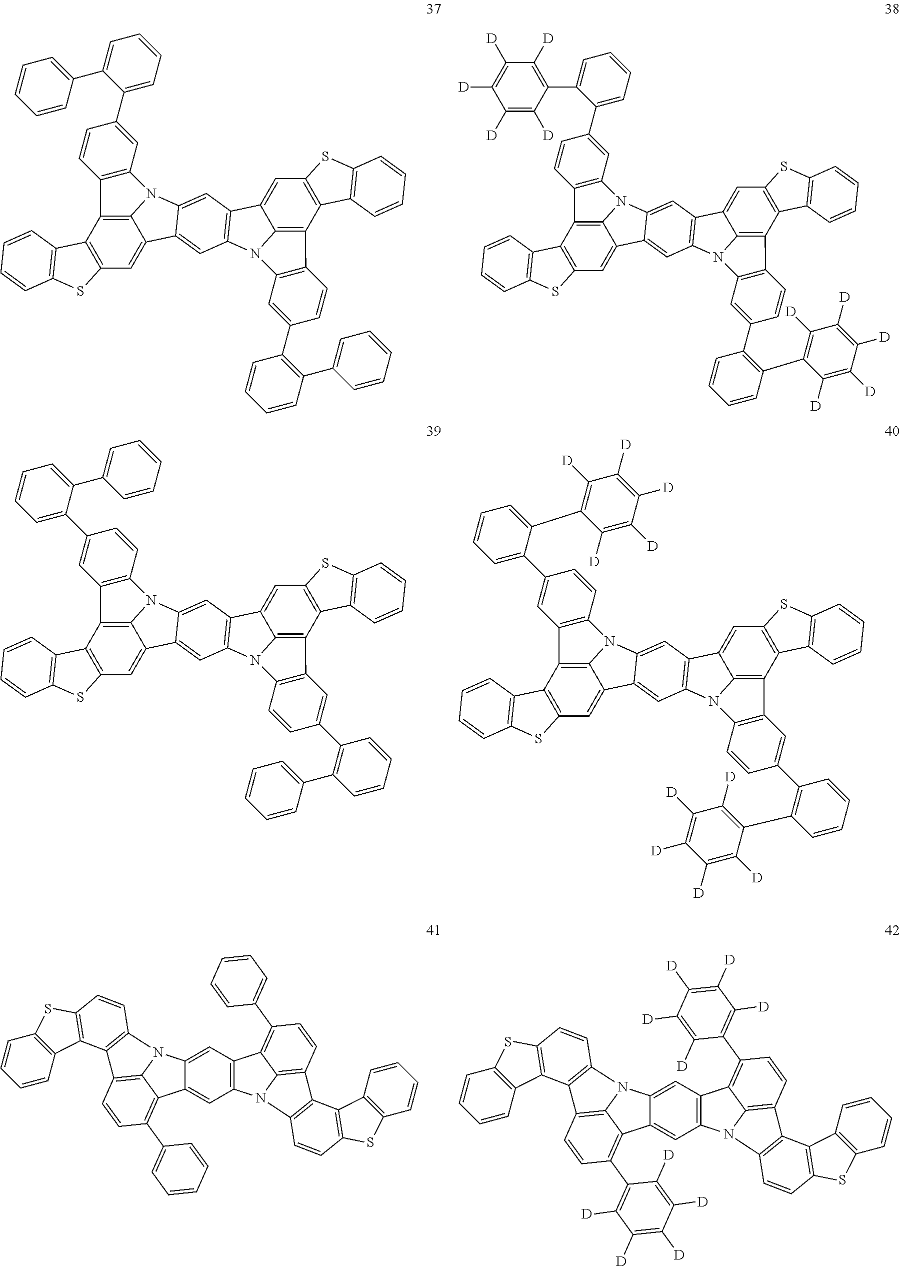 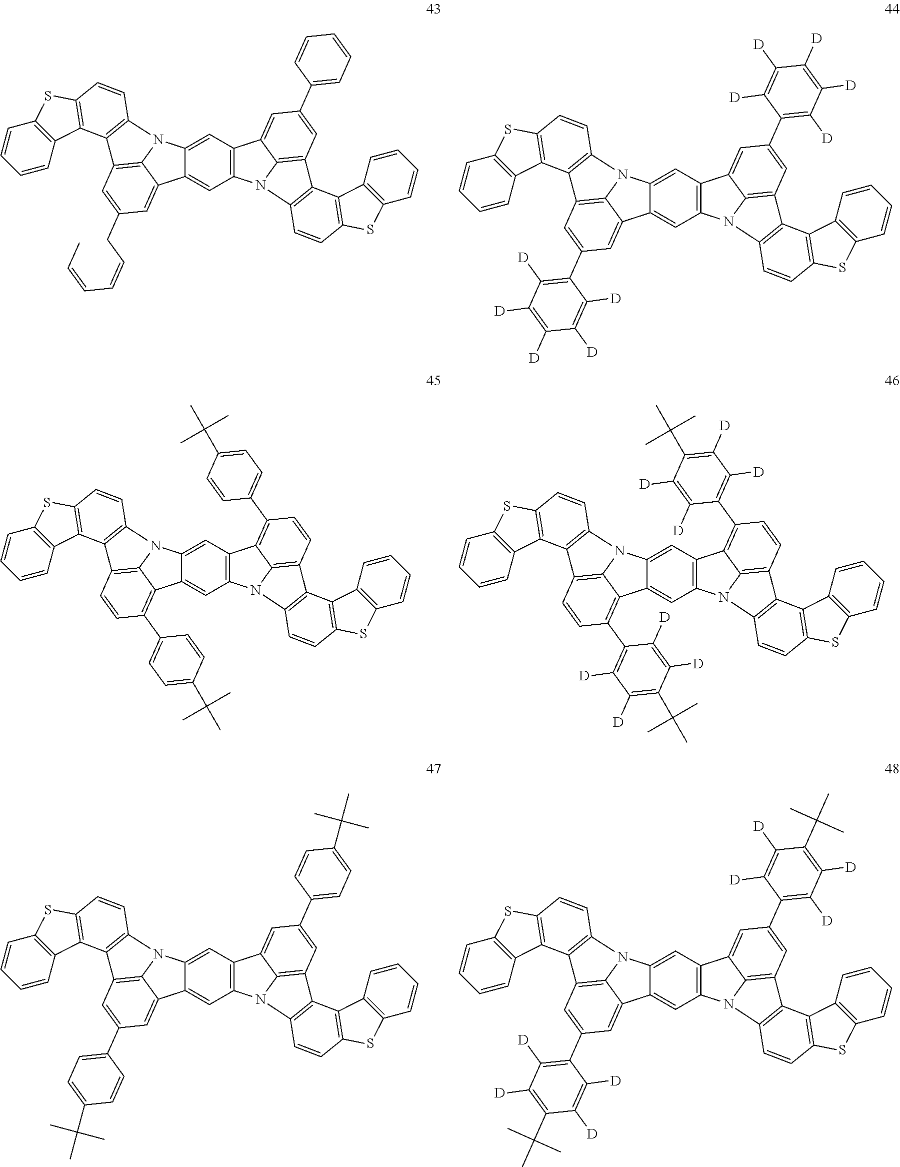 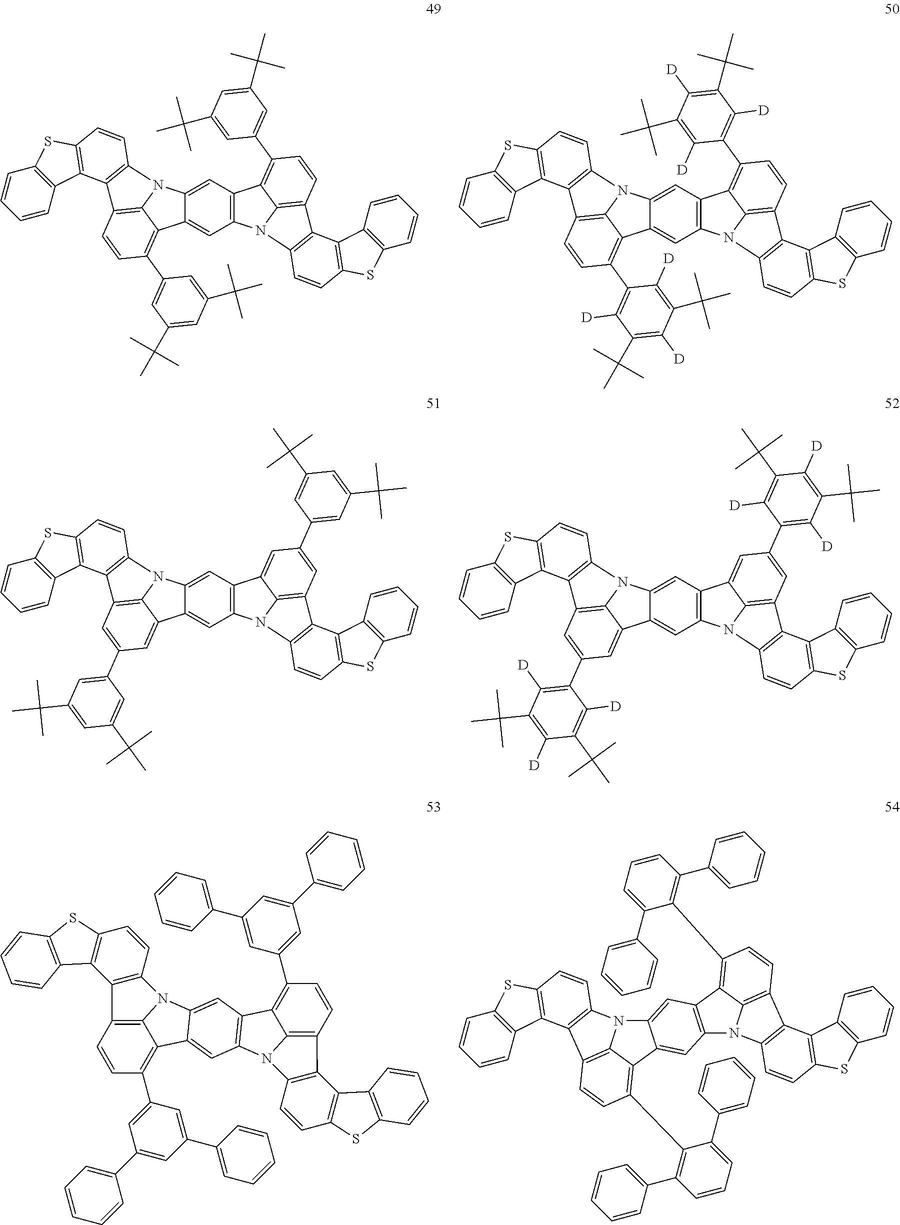 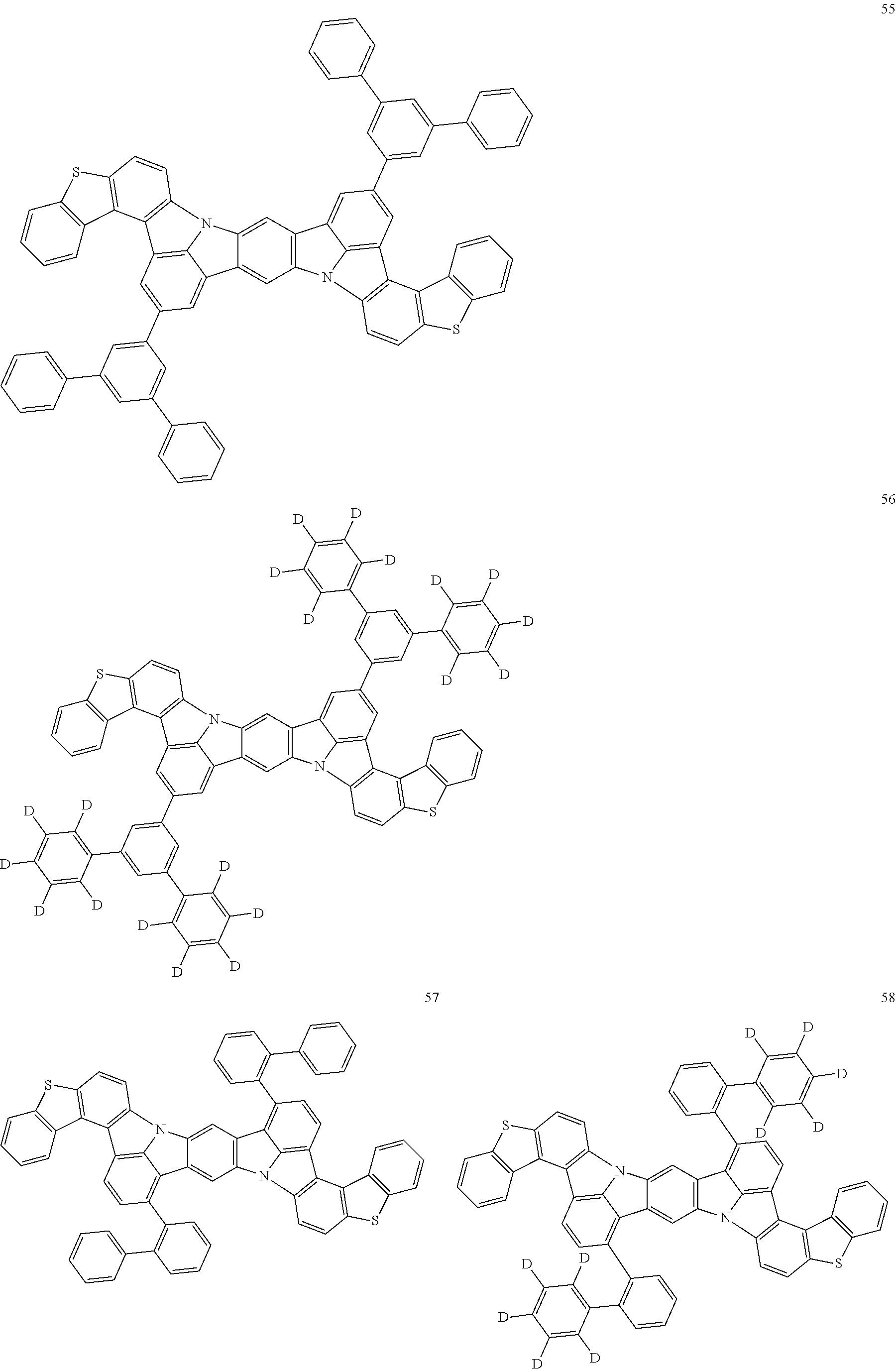 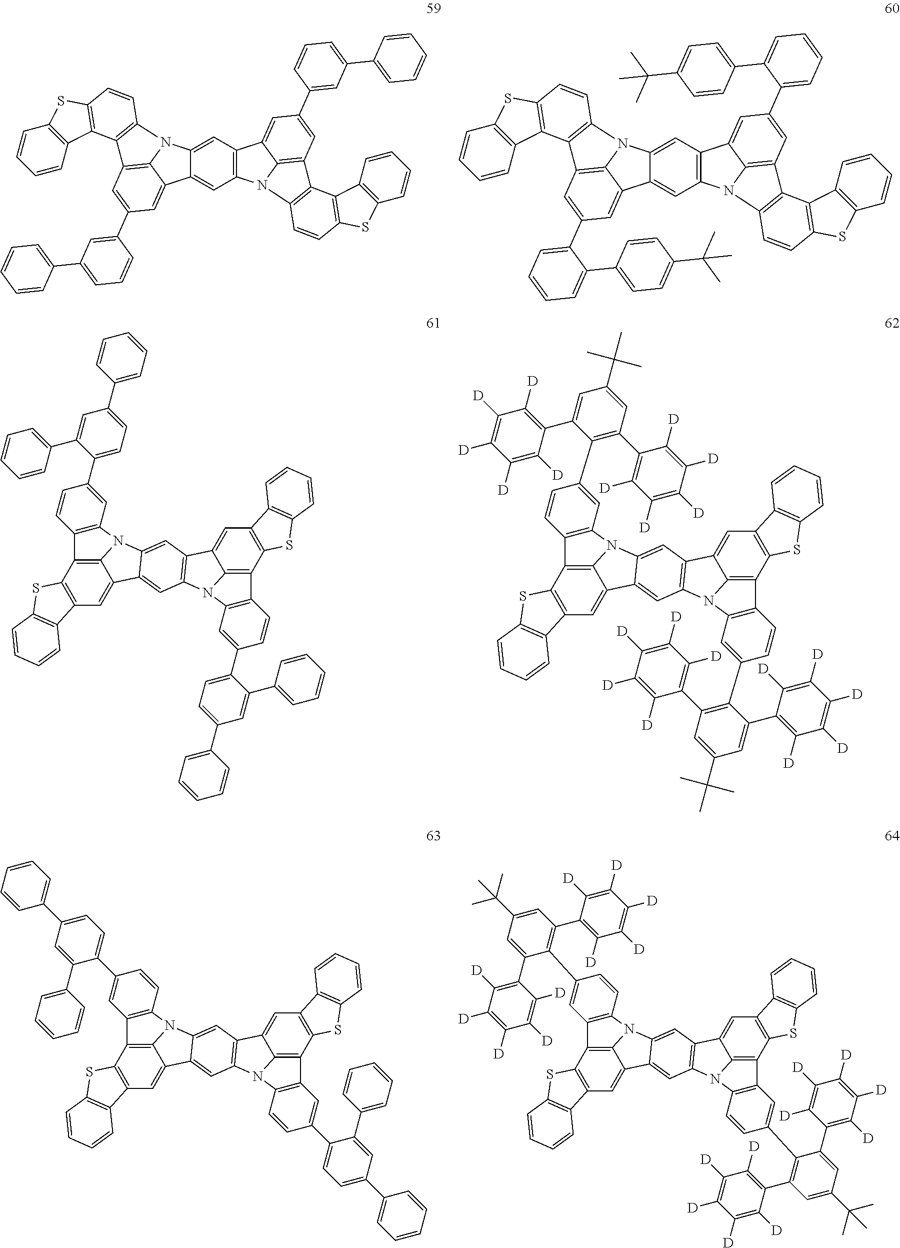 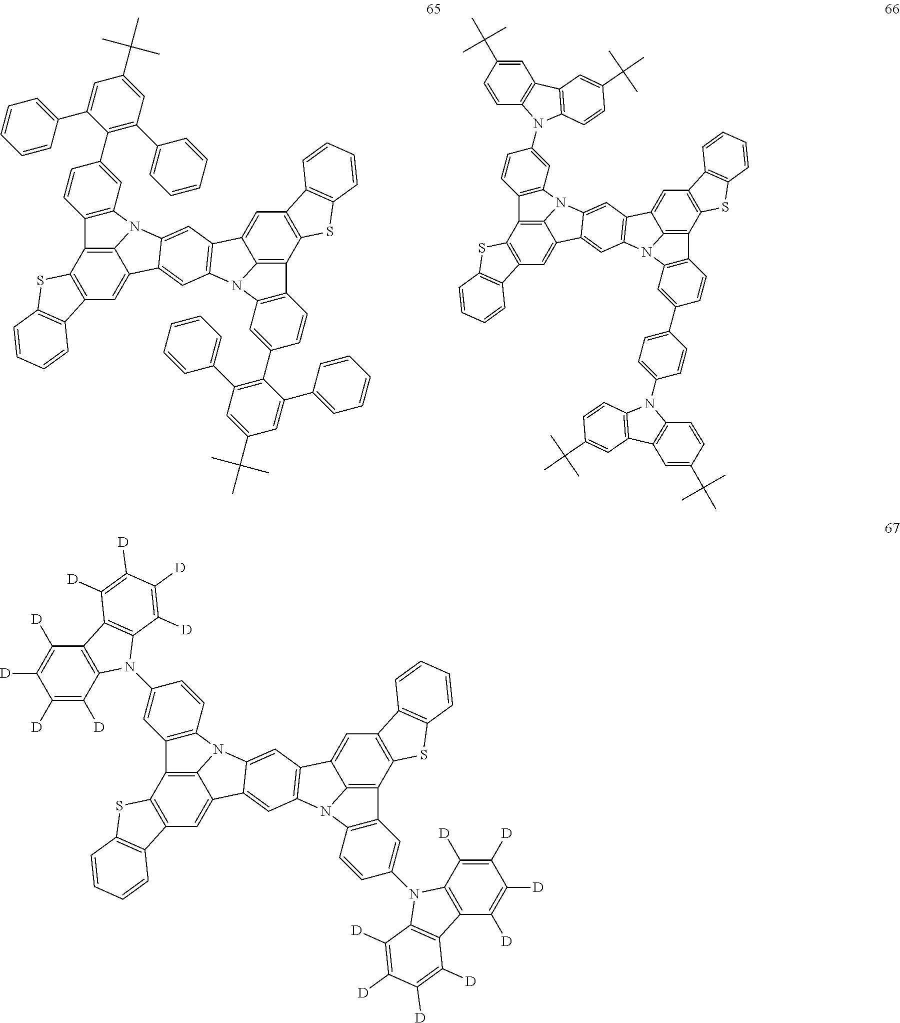 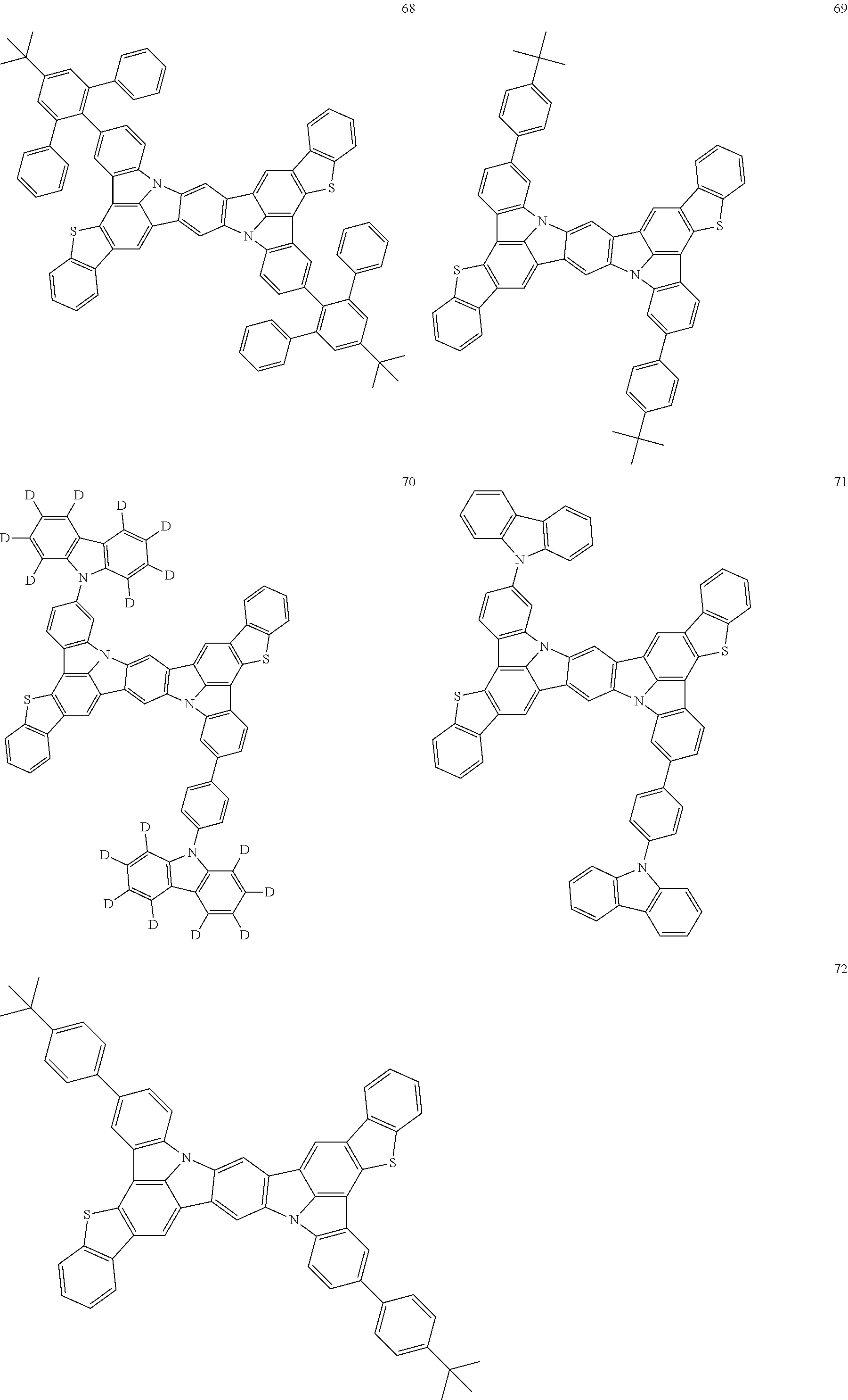 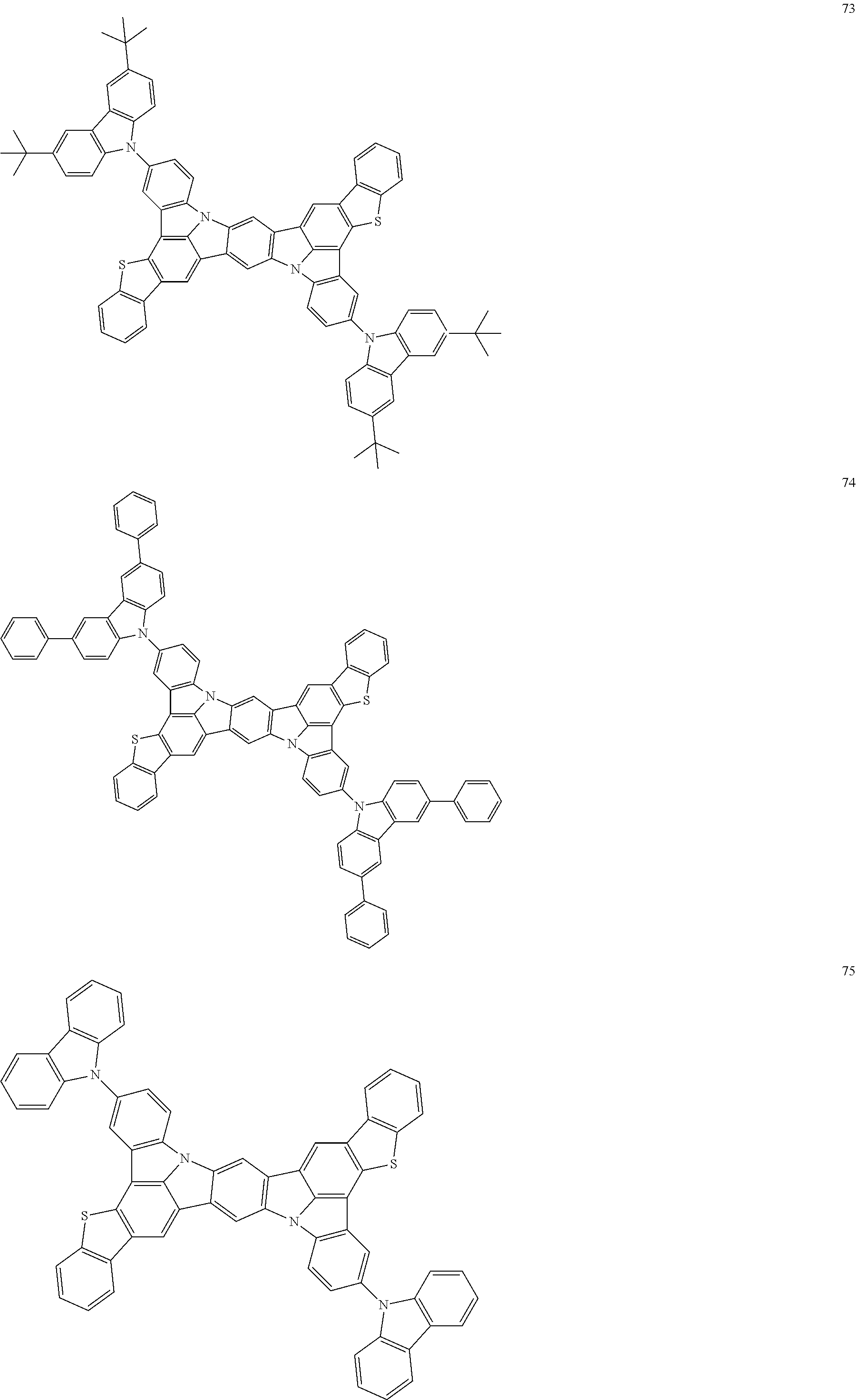 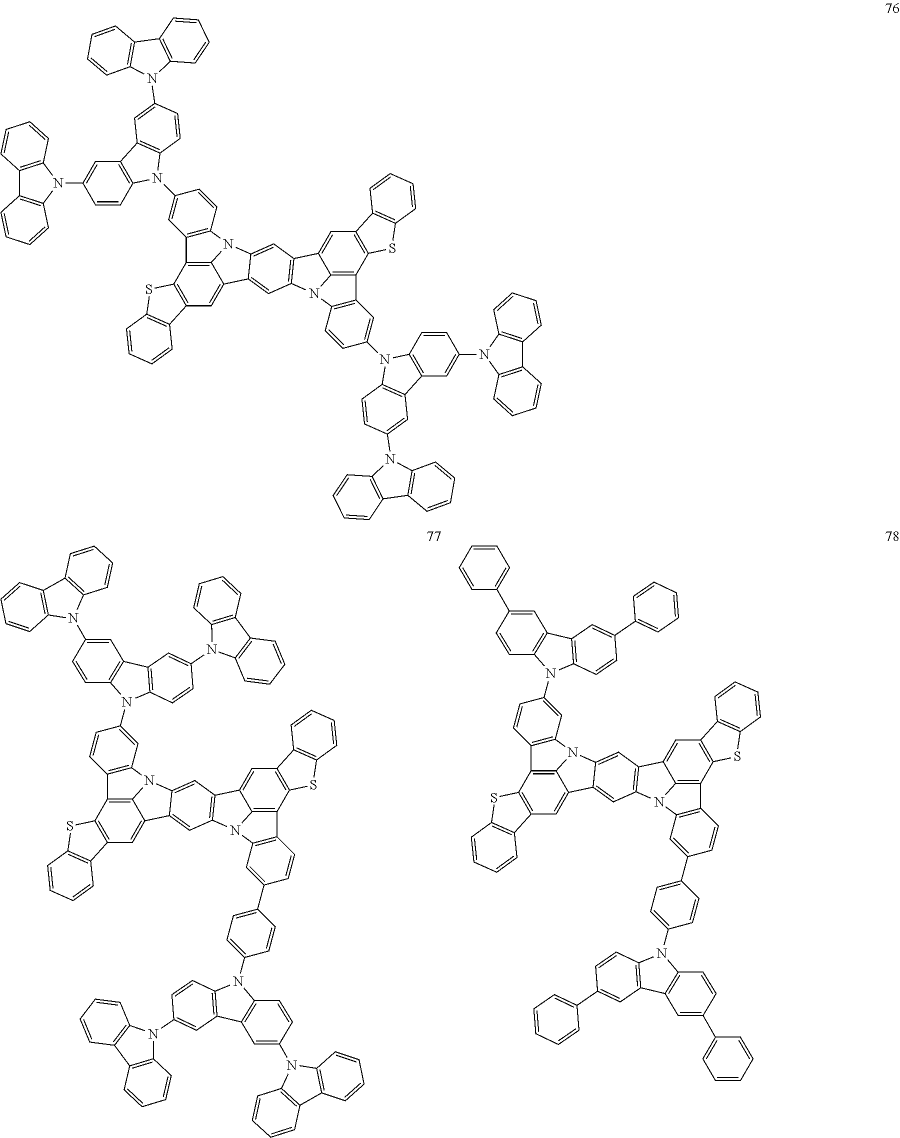 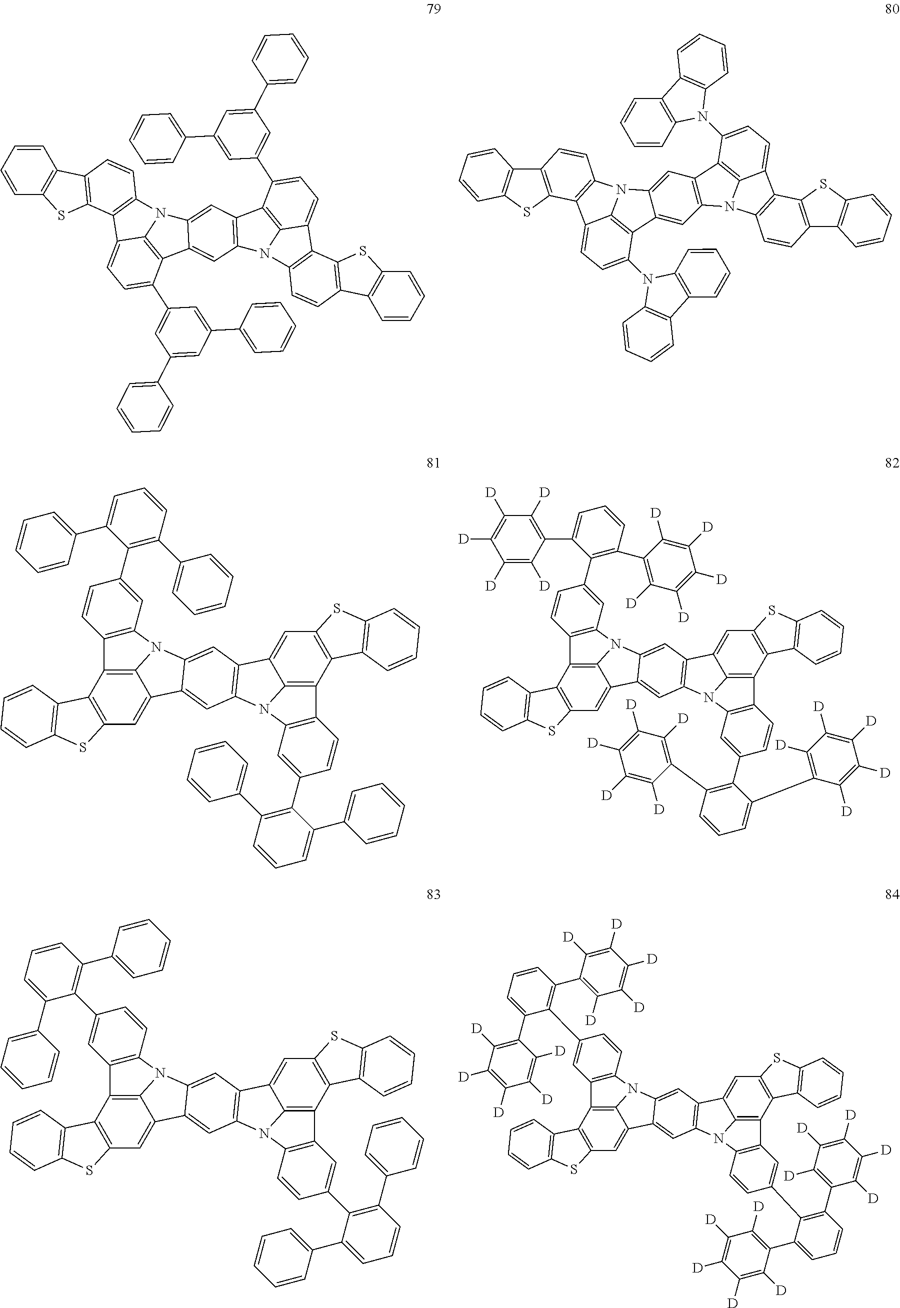 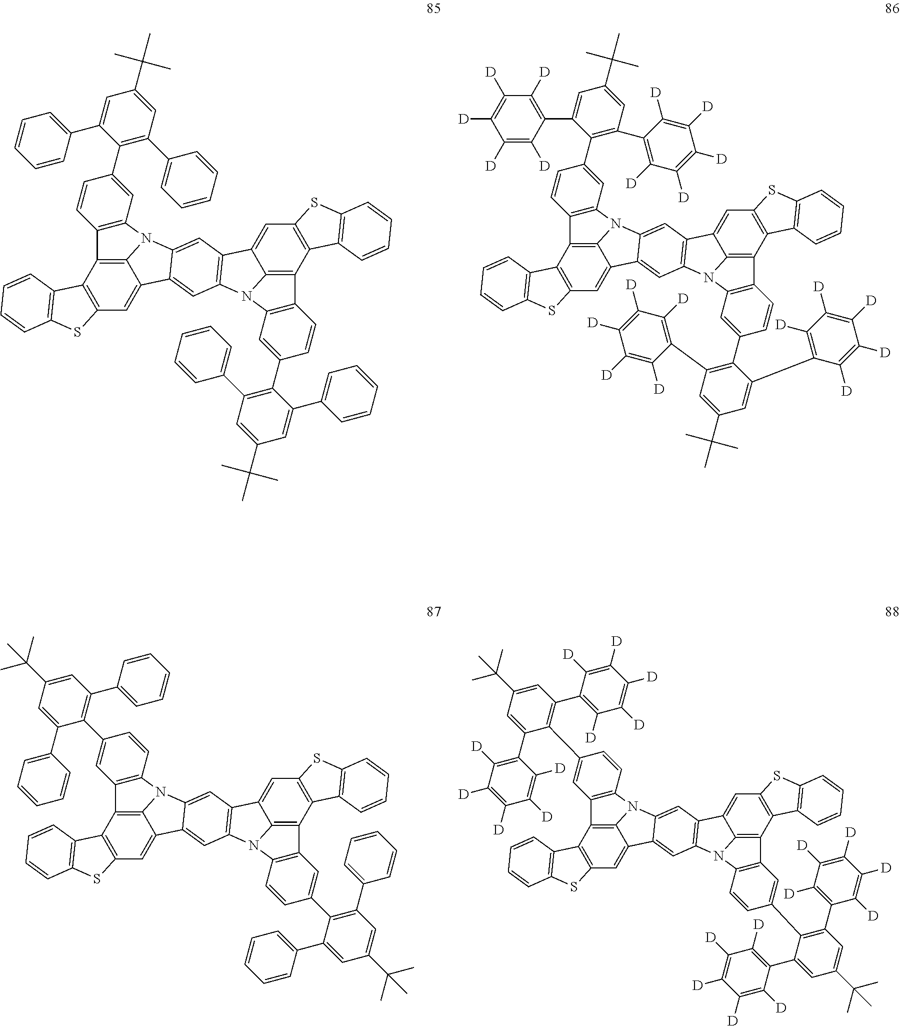 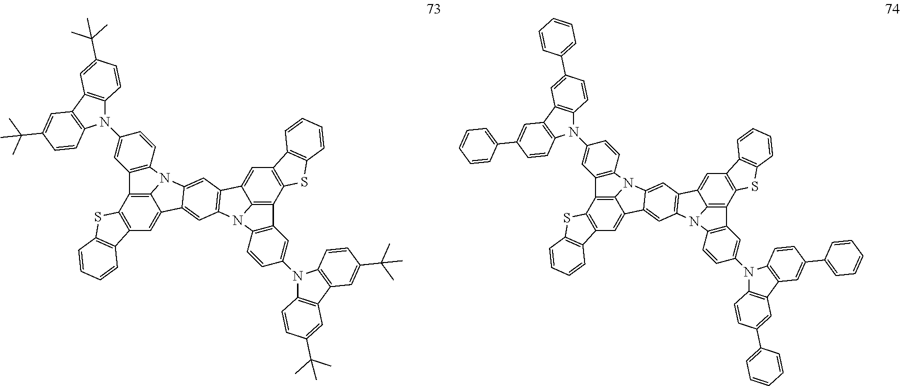 [SMILES: CC(C)(C)c1ccc2c(c1)c1cc(C(C)(C)C)ccc1n2-c1ccc2c(c1)c1c3sc4ccccc4c3cc3c4cc5c(cc4n2c31)c1cc2c3ccccc3sc2c2c3cc(-n4c6ccc(C(C)(C)C)cc6c6cc(C(C)(C)C)ccc64)ccc3n5c12 ; c1ccc(-c2ccc3c(c2)c2cc(-c4ccccc4)ccc2n3-c2ccc3c(c2)c2c4sc5ccccc5c4cc4c5cc6c(cc5n3c42)c2cc3c4ccccc4sc3c3c4cc(-n5c7ccc(-c8ccccc8)cc7c7cc(-c8ccccc8)ccc75)ccc4n6c23)cc1] 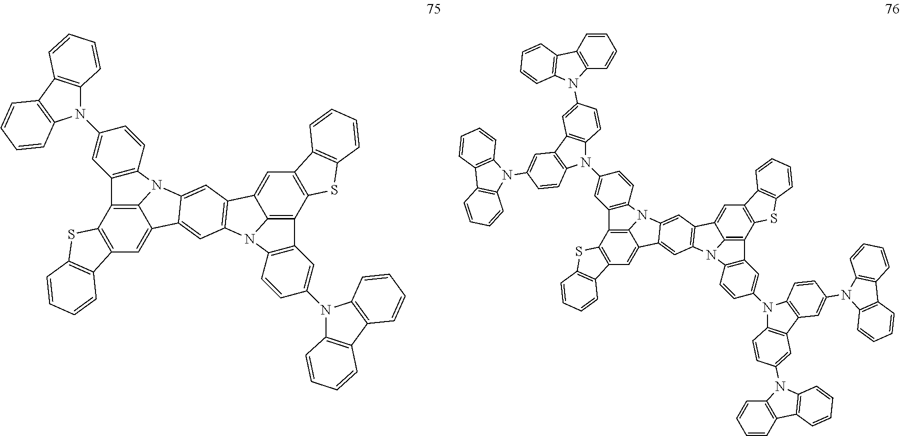 [SMILES: c1ccc2c(c1)sc1c2cc2c3cc4c(cc3n3c5ccc(-n6c7ccccc7c7ccccc76)cc5c1c23)c1cc2c3ccccc3sc2c2c3cc(-n5c6ccccc6c6ccccc65)ccc3n4c12 ; c1ccc2c(c1)sc1c2cc2c3cc4c(cc3n3c5ccc(-n6c7ccc(-n8c9ccccc9c9ccccc98)cc7c7cc(-n8c9ccccc9c9ccccc98)ccc76)cc5c1c23)c1cc2c3ccccc3sc2c2c3cc(-n5c6ccc(-n7c8ccccc8c8ccccc87)cc6c6cc(-n7c8ccccc8c8ccccc87)ccc65)ccc3n4c12] 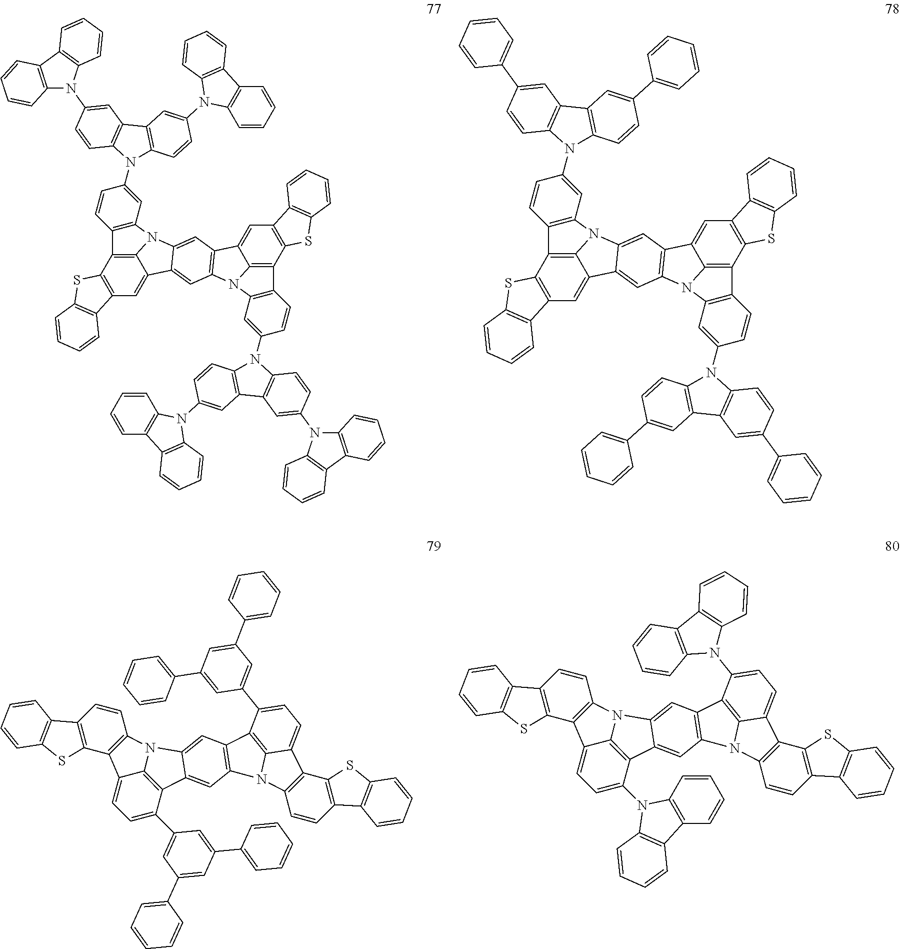 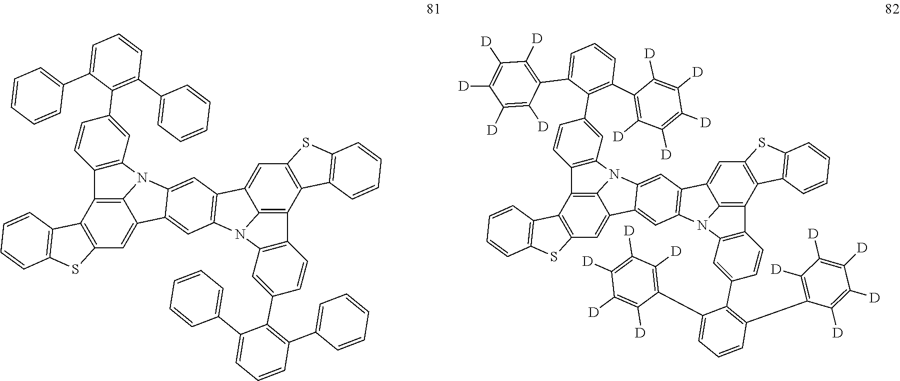 [SMILES: c1ccc(-c2cccc(-c3ccccc3)c2-c2ccc3c4c5c(cc6c7cc8c(cc7n(c3c2)c64)c2cc3sc4ccccc4c3c3c4ccc(-c6c(-c7ccccc7)cccc6-c6ccccc6)cc4n8c23)sc2ccccc25)cc1 ; [2H]c1c([2H])c([2H])c(-c2cccc(-c3c([2H])c([2H])c([2H])c([2H])c3[2H])c2-c2ccc3c4c5c(cc6c7cc8c(cc7n(c3c2)c64)c2cc3sc4ccccc4c3c3c4ccc(-c6c(-c7c([2H])c([2H])c([2H])c([2H])c7[2H])cccc6-c6c([2H])c([2H])c([2H])c([2H])c6[2H])cc4n8c23)sc2ccccc25)c([2H])c1[2H]] 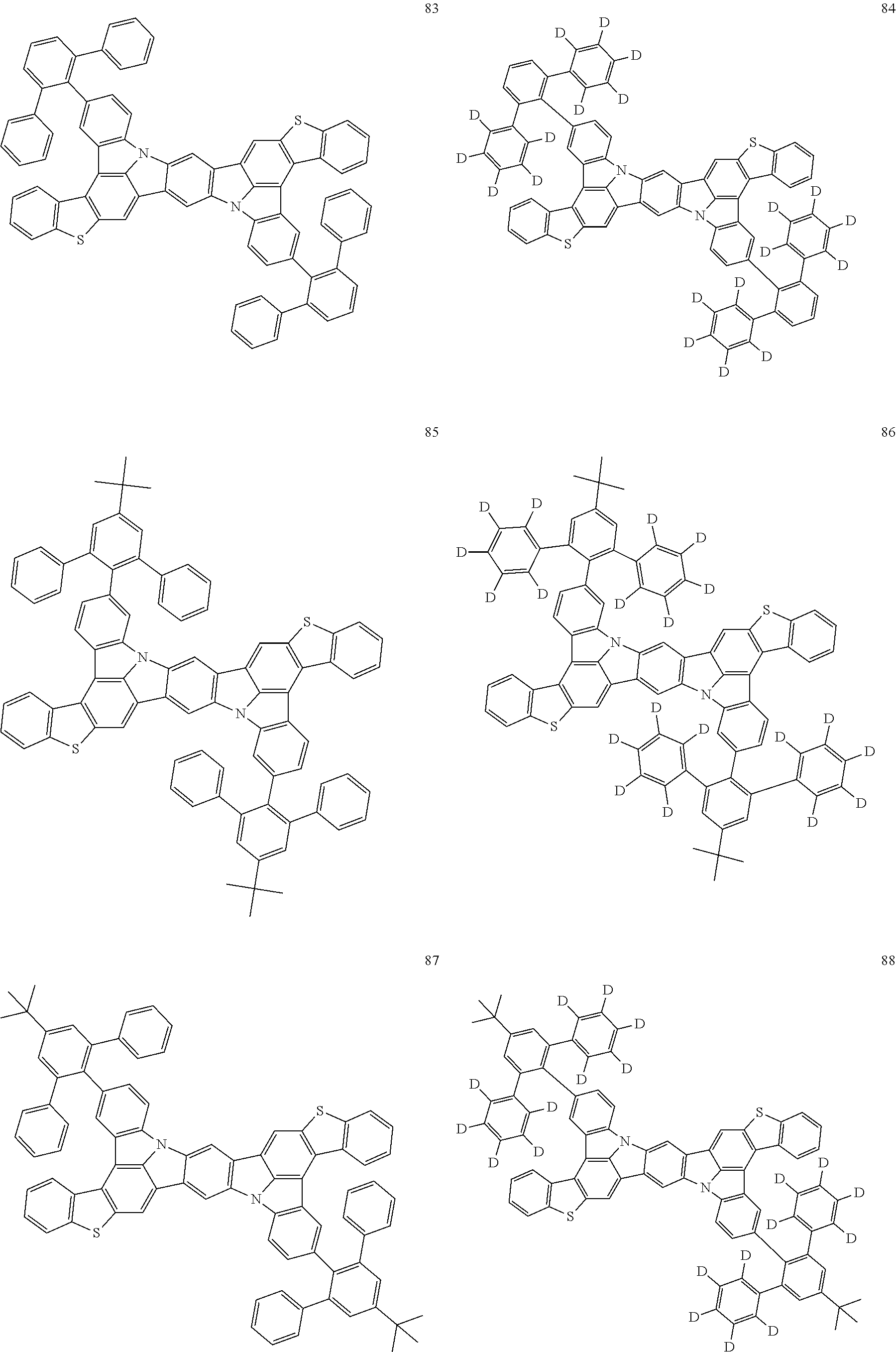 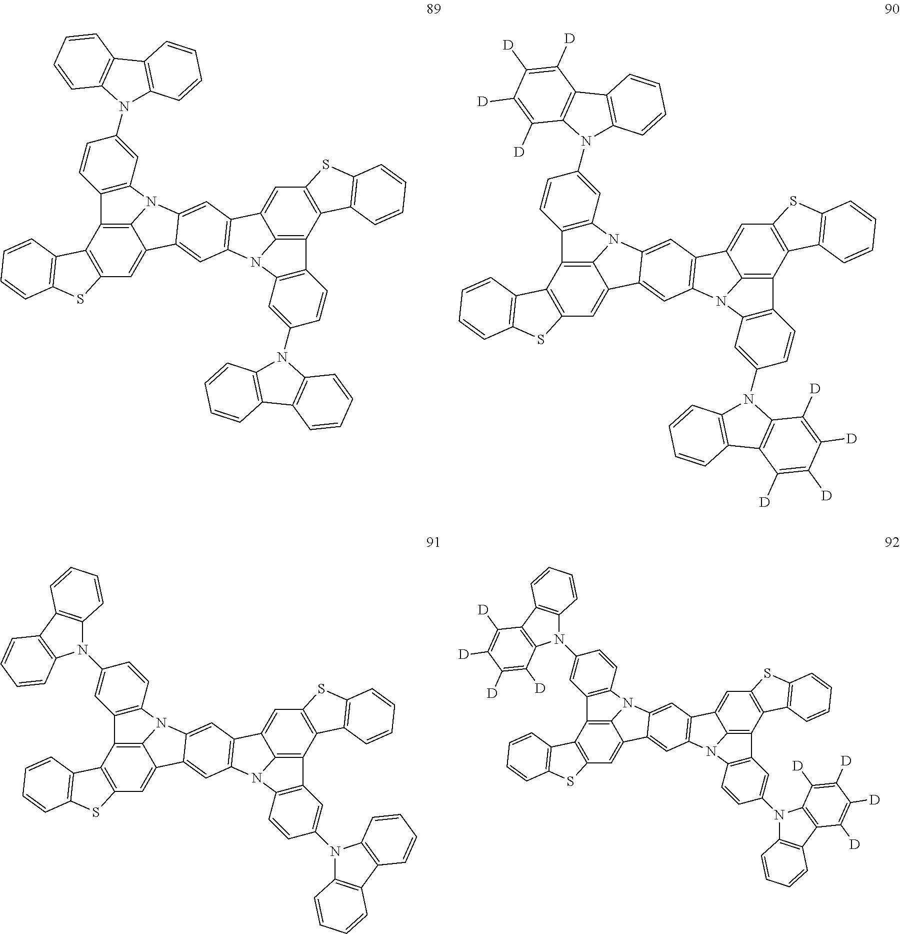 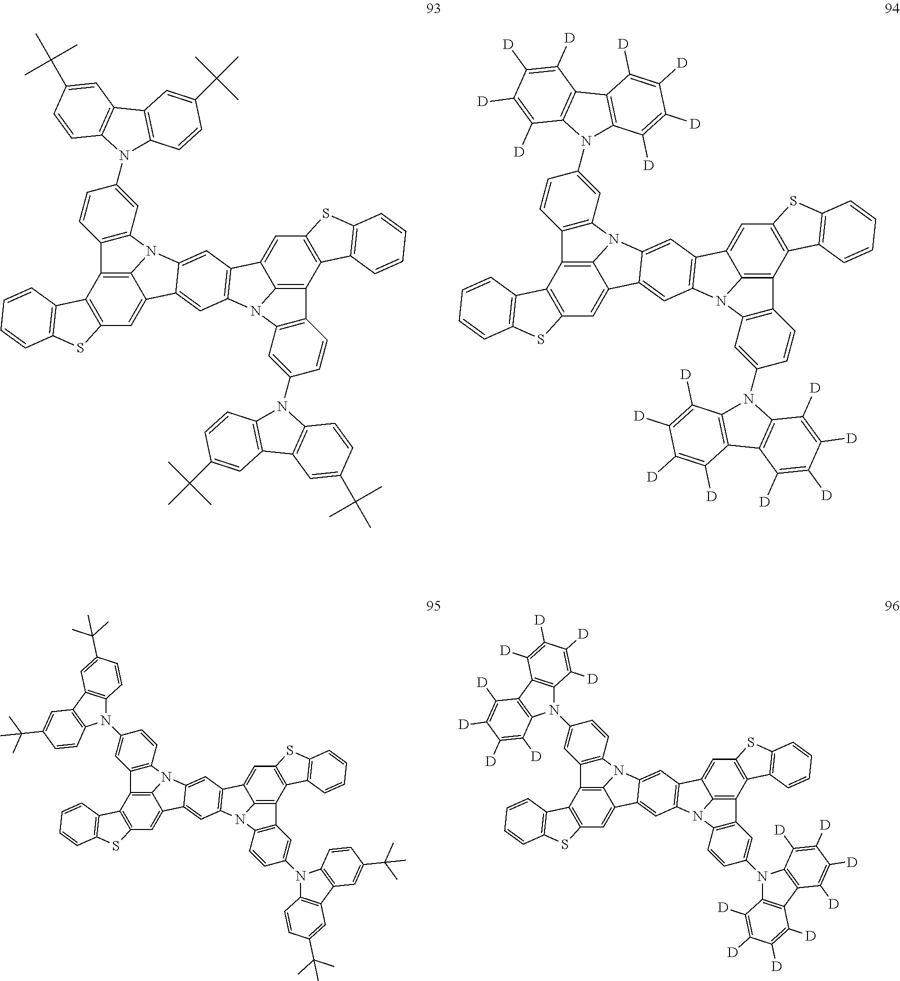 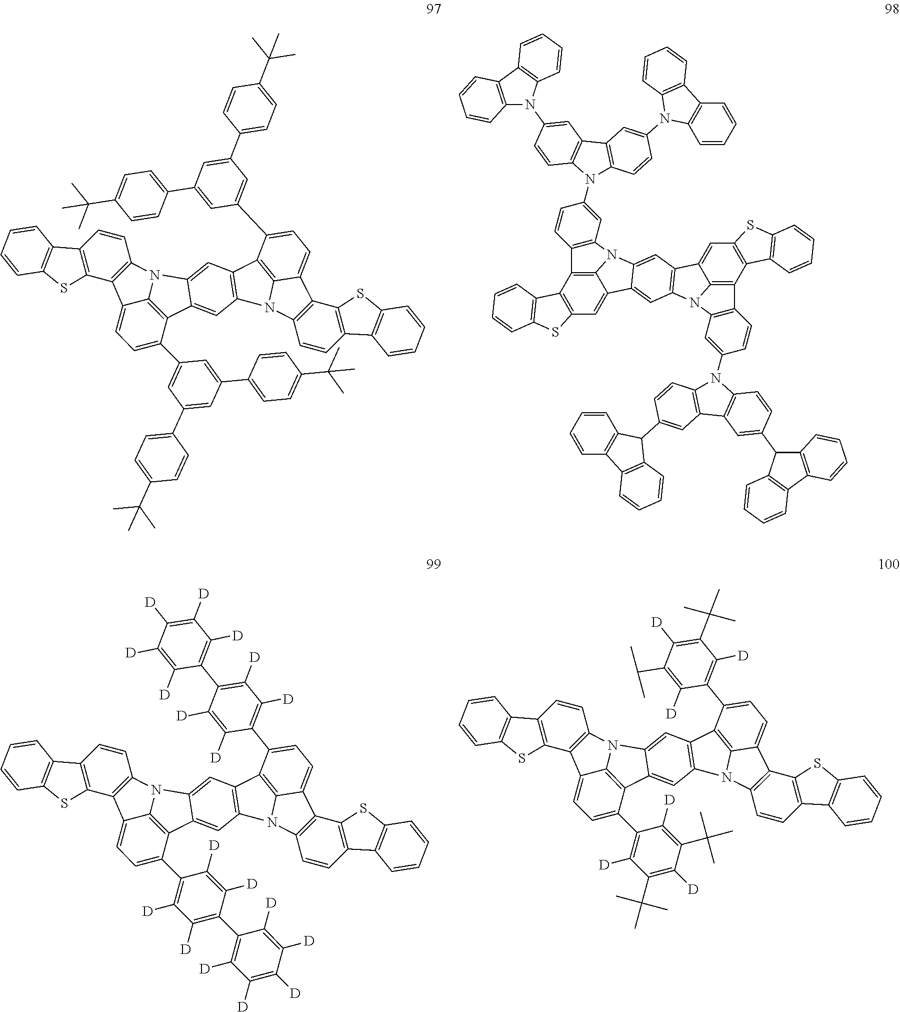 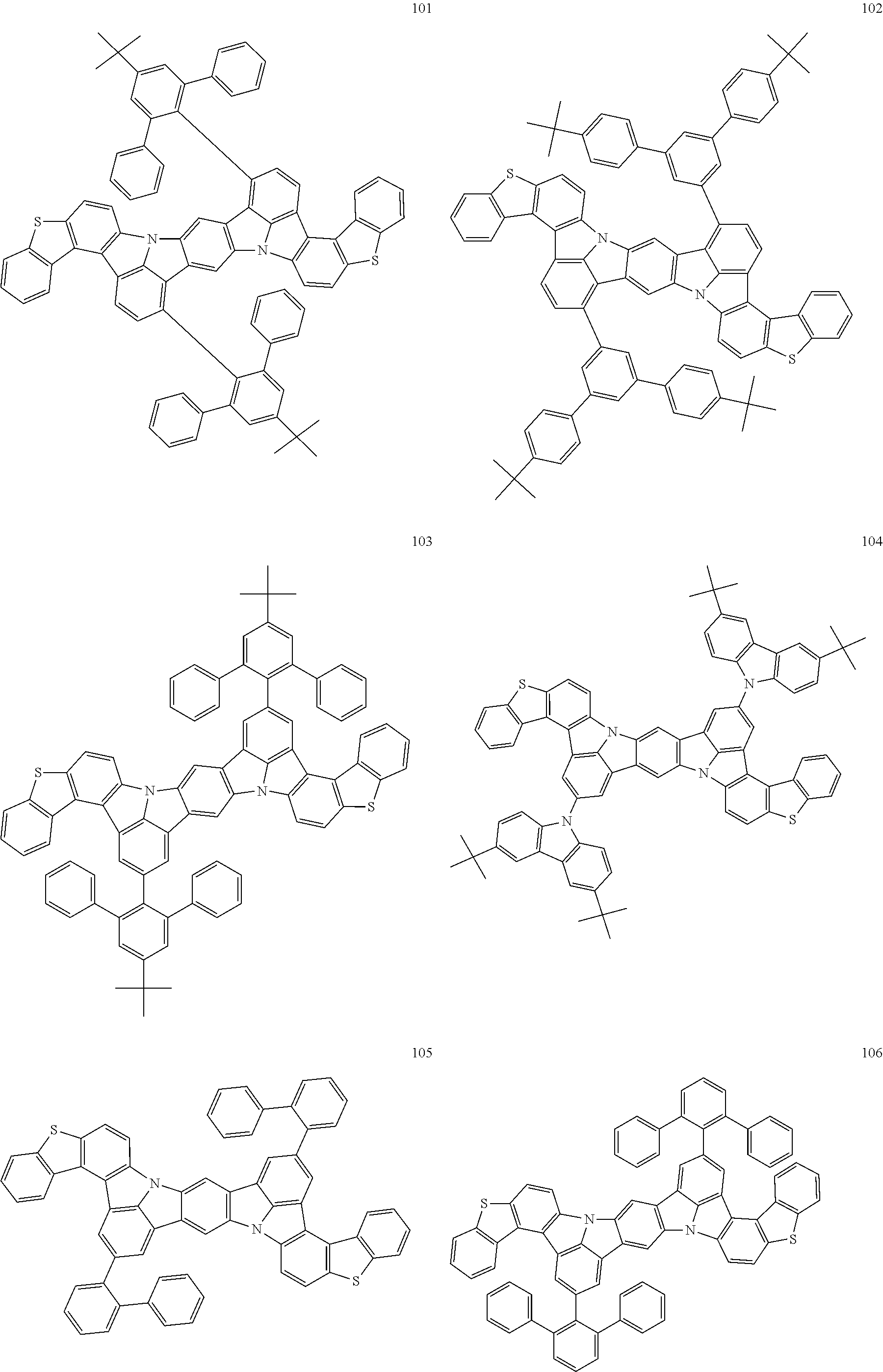 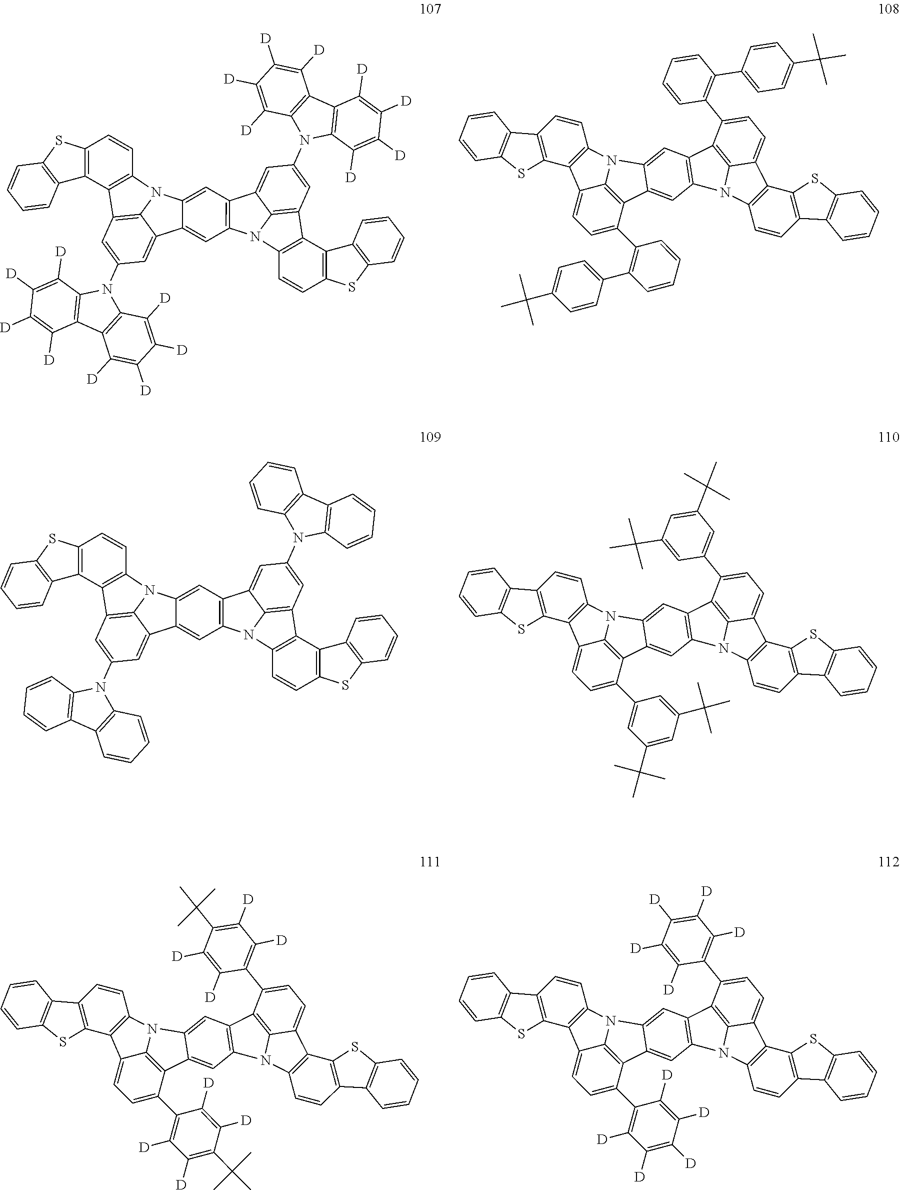 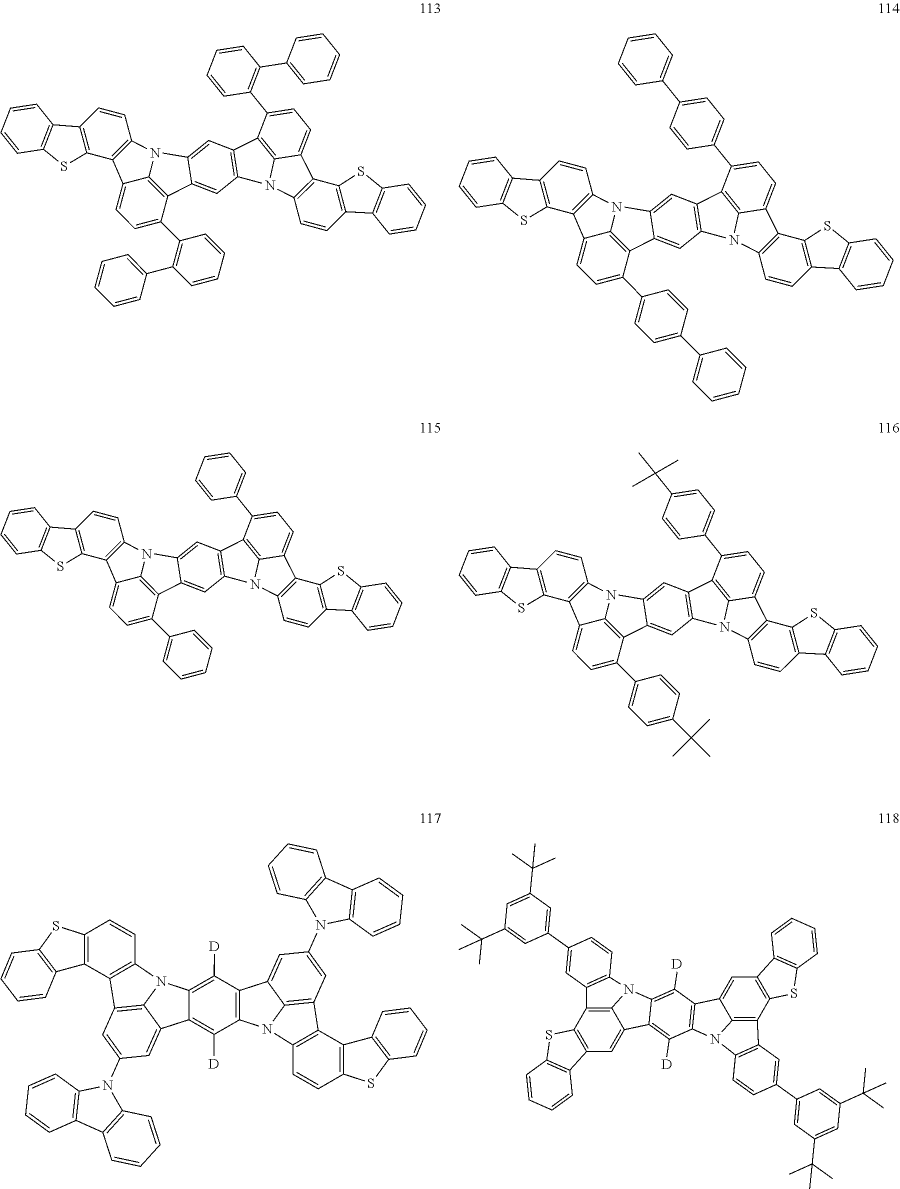 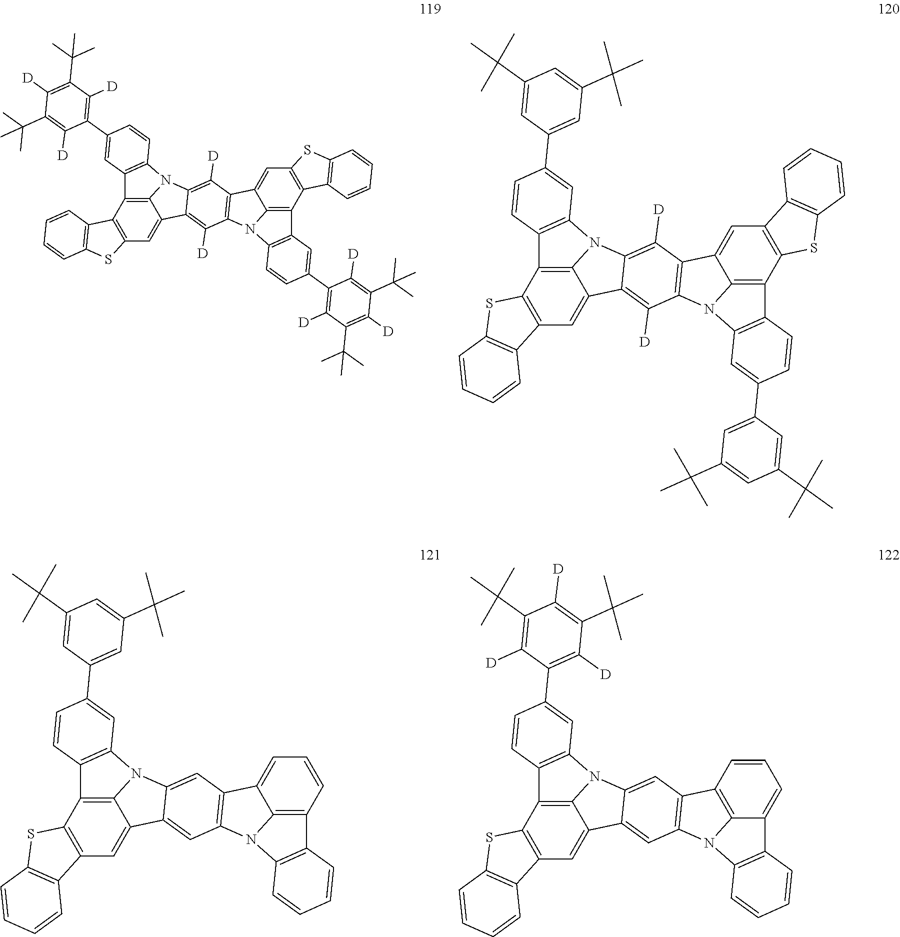 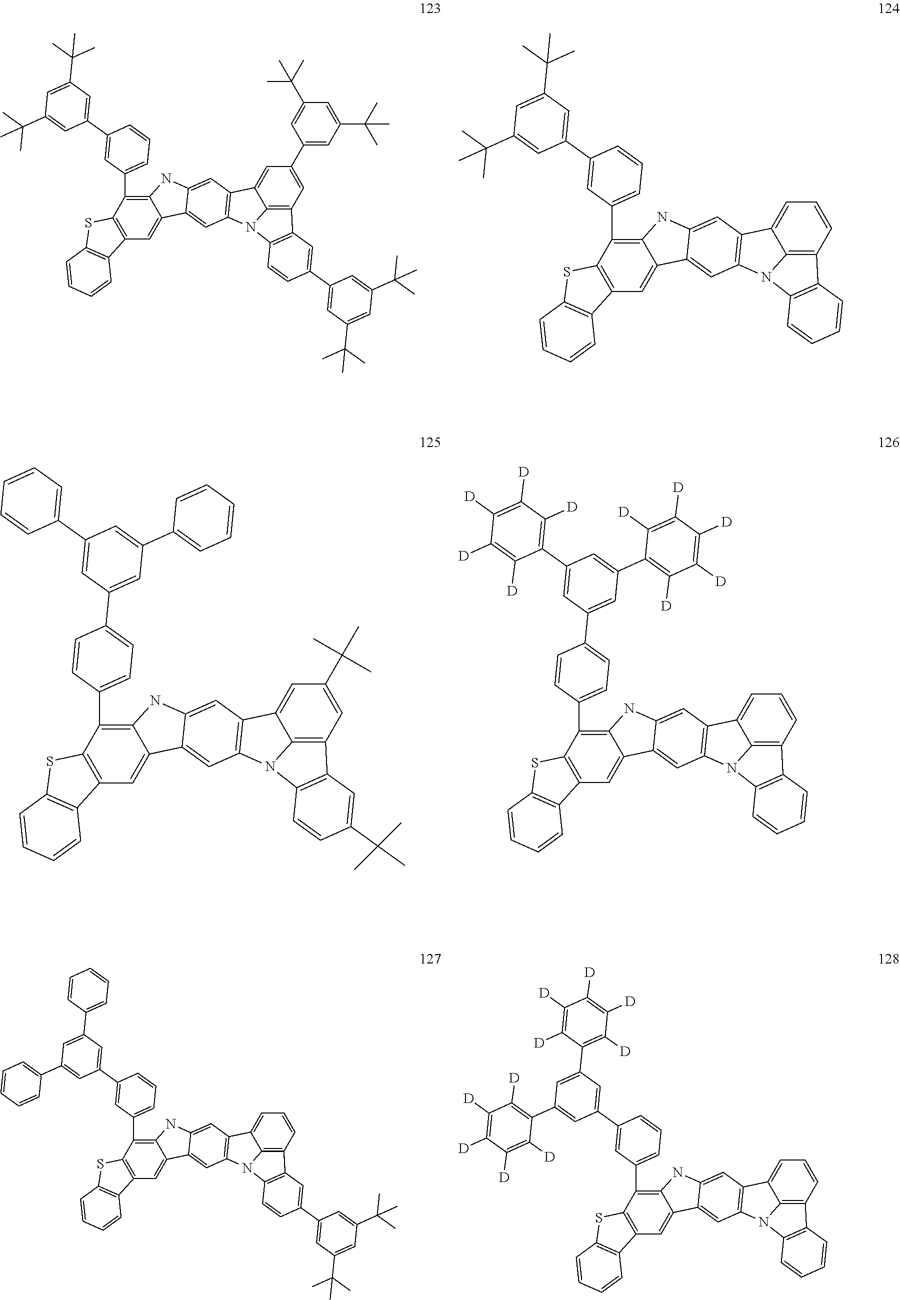 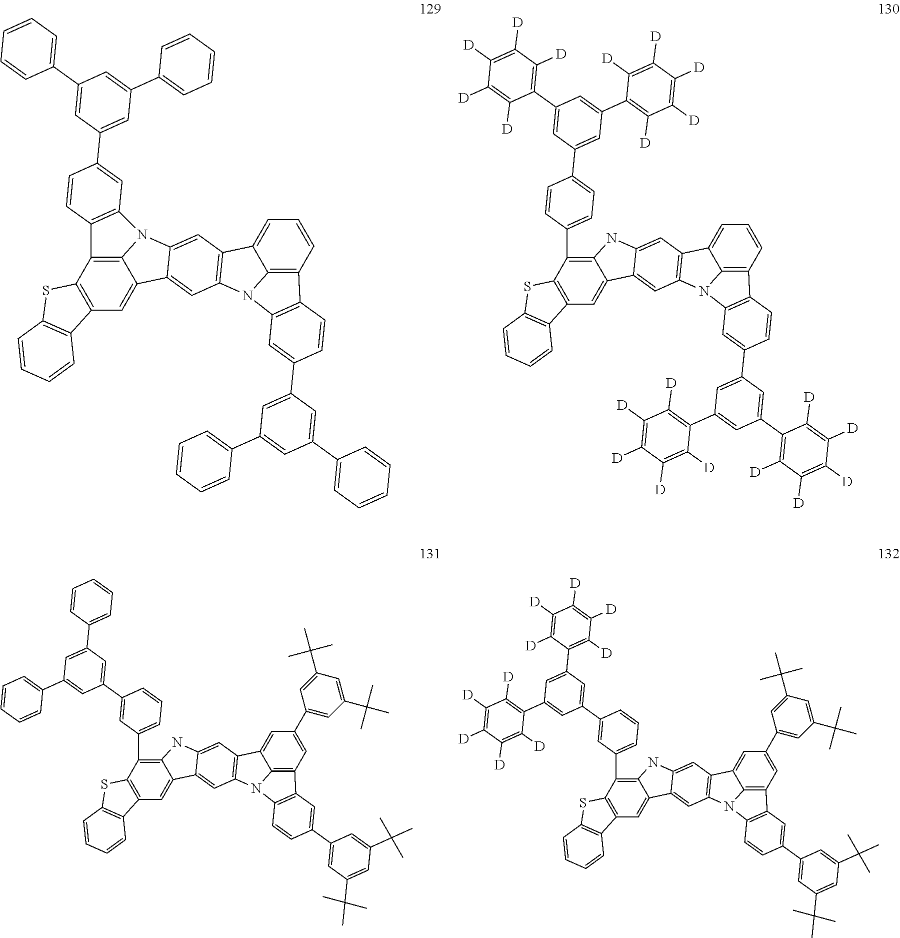 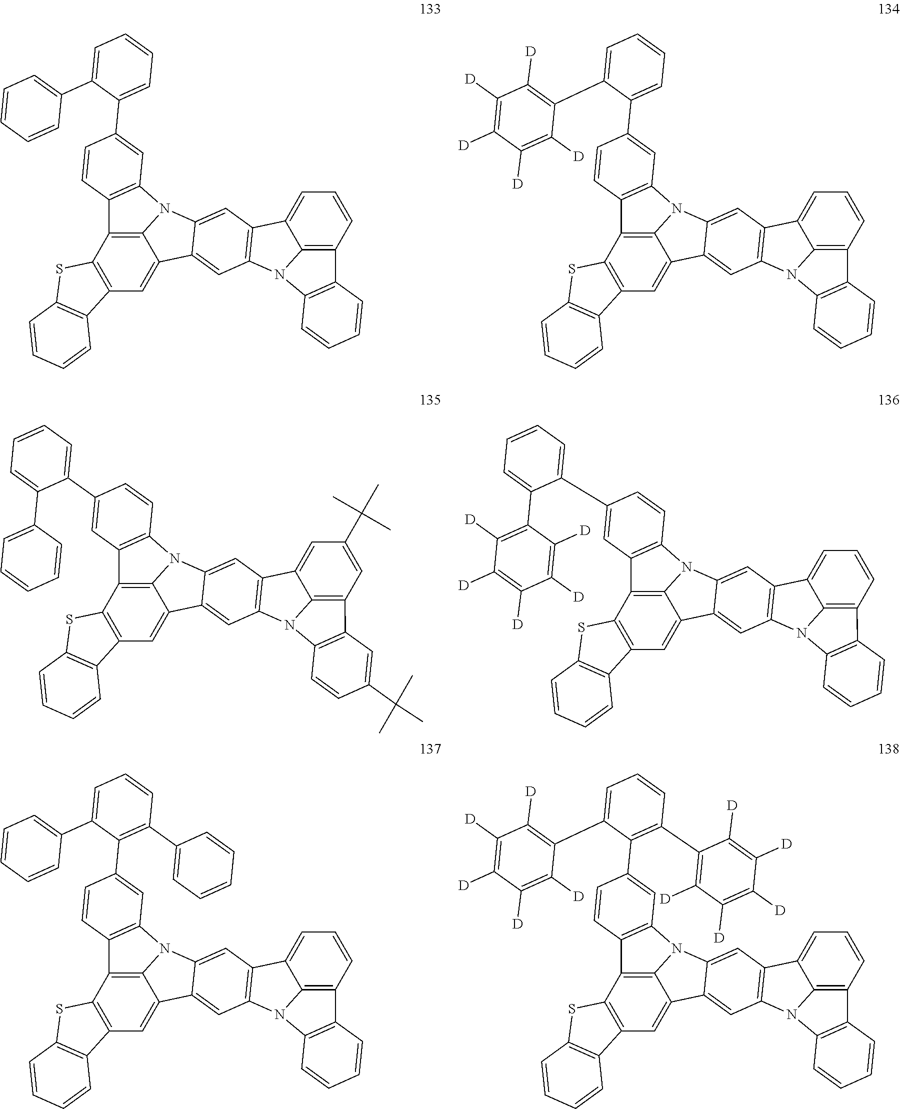 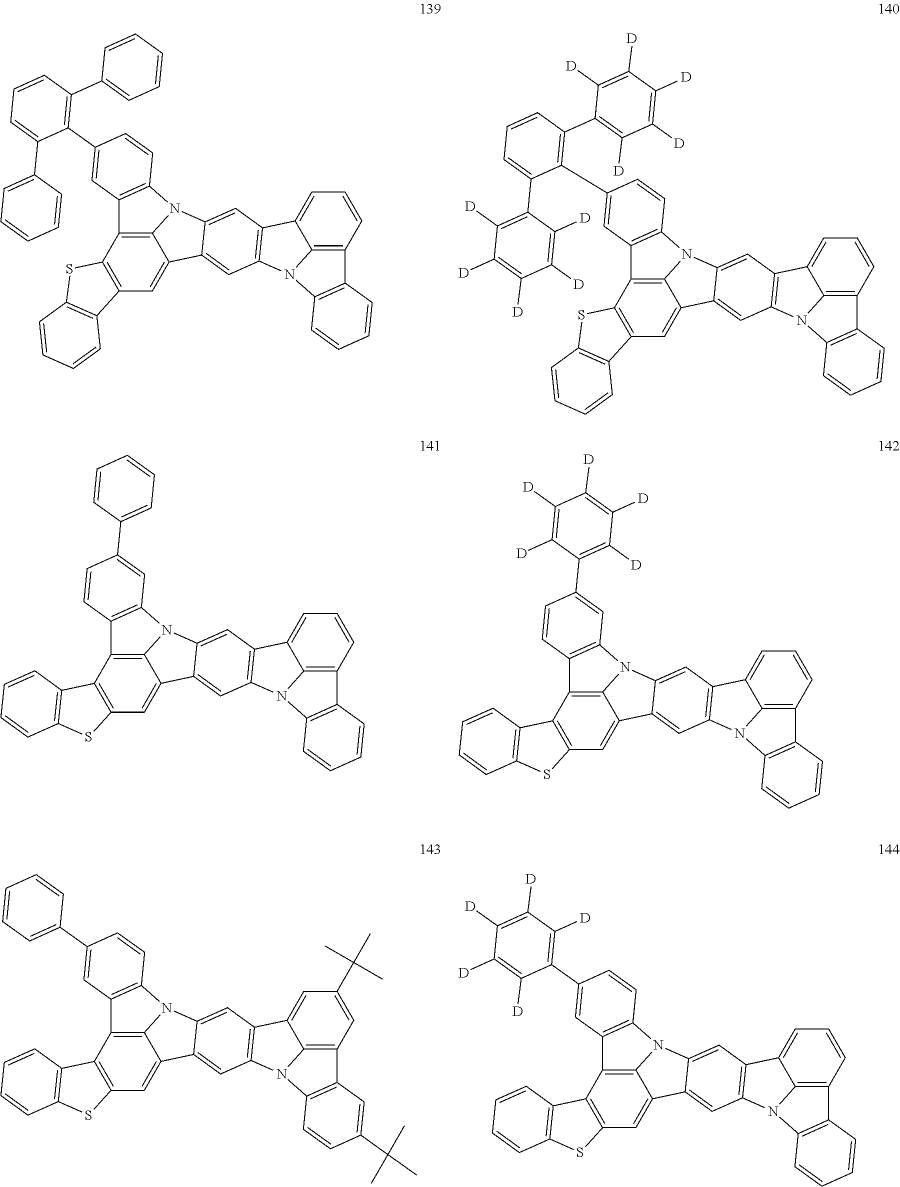 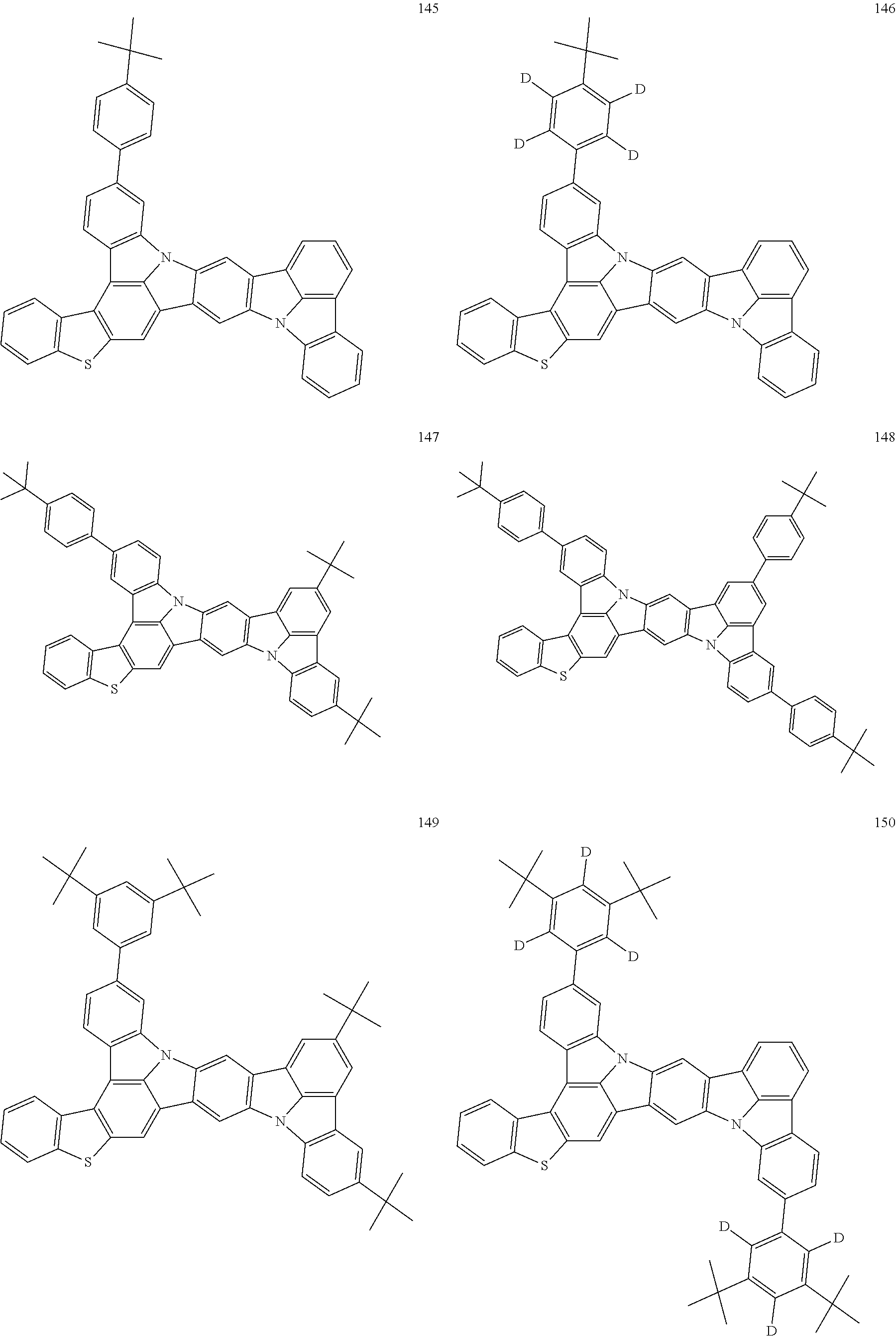 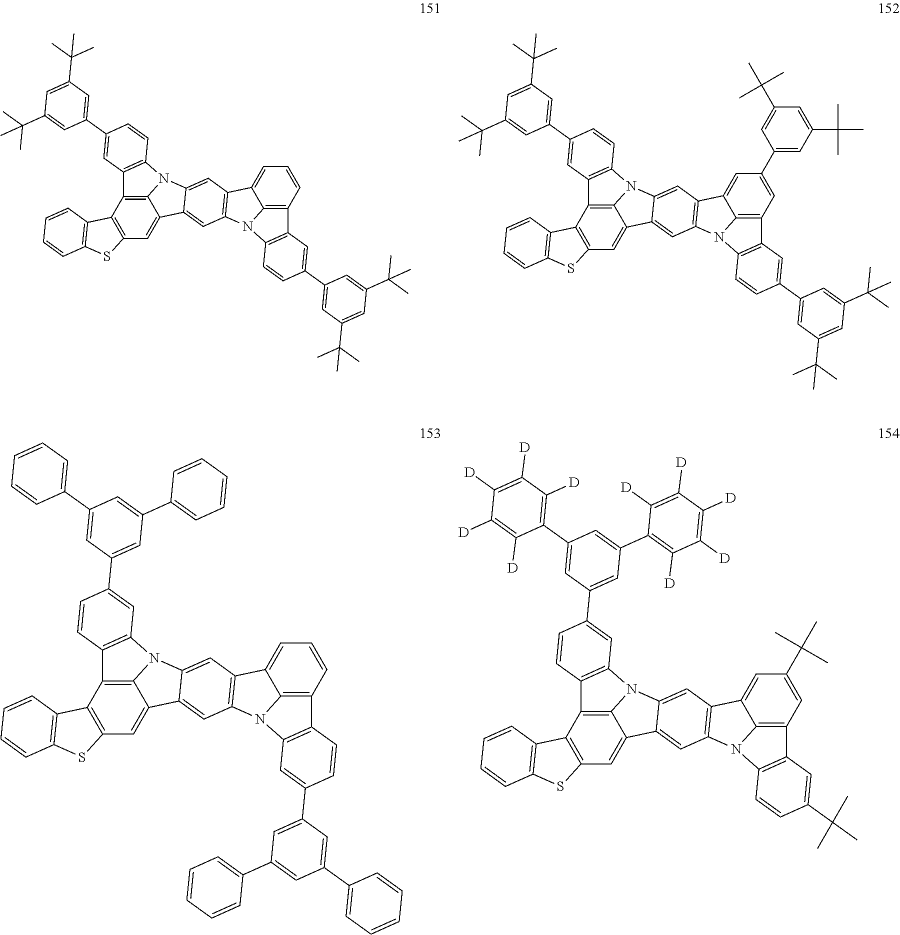 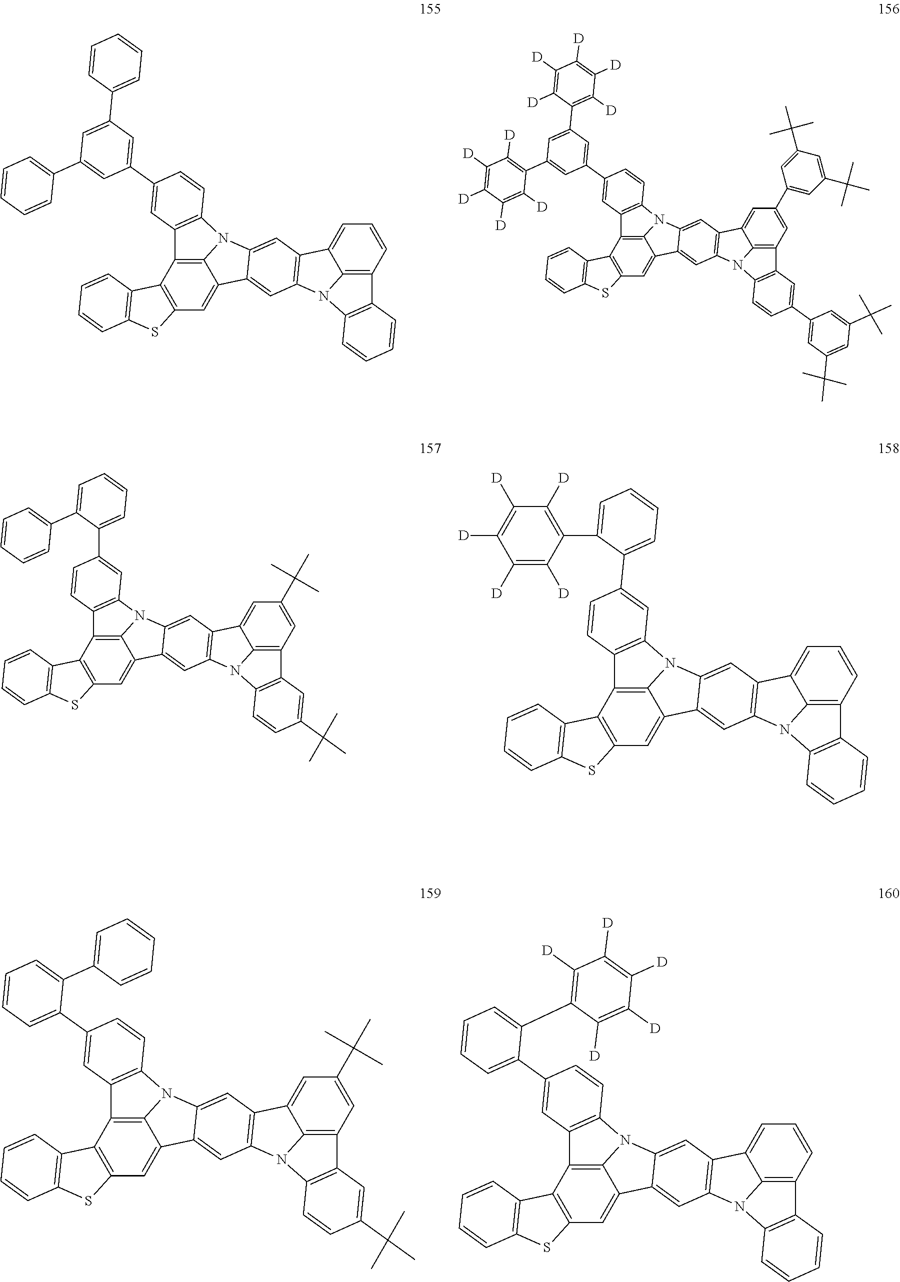 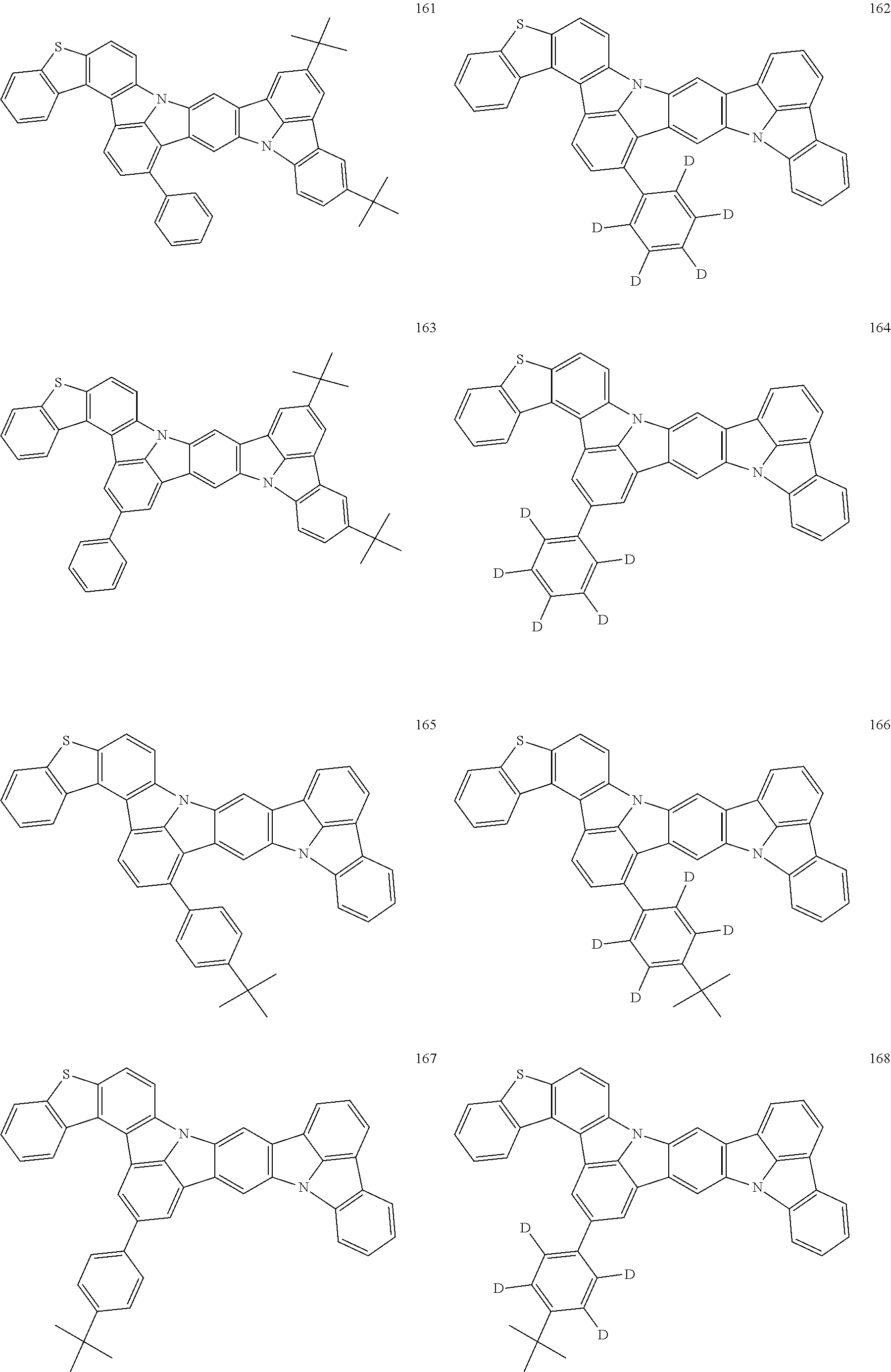 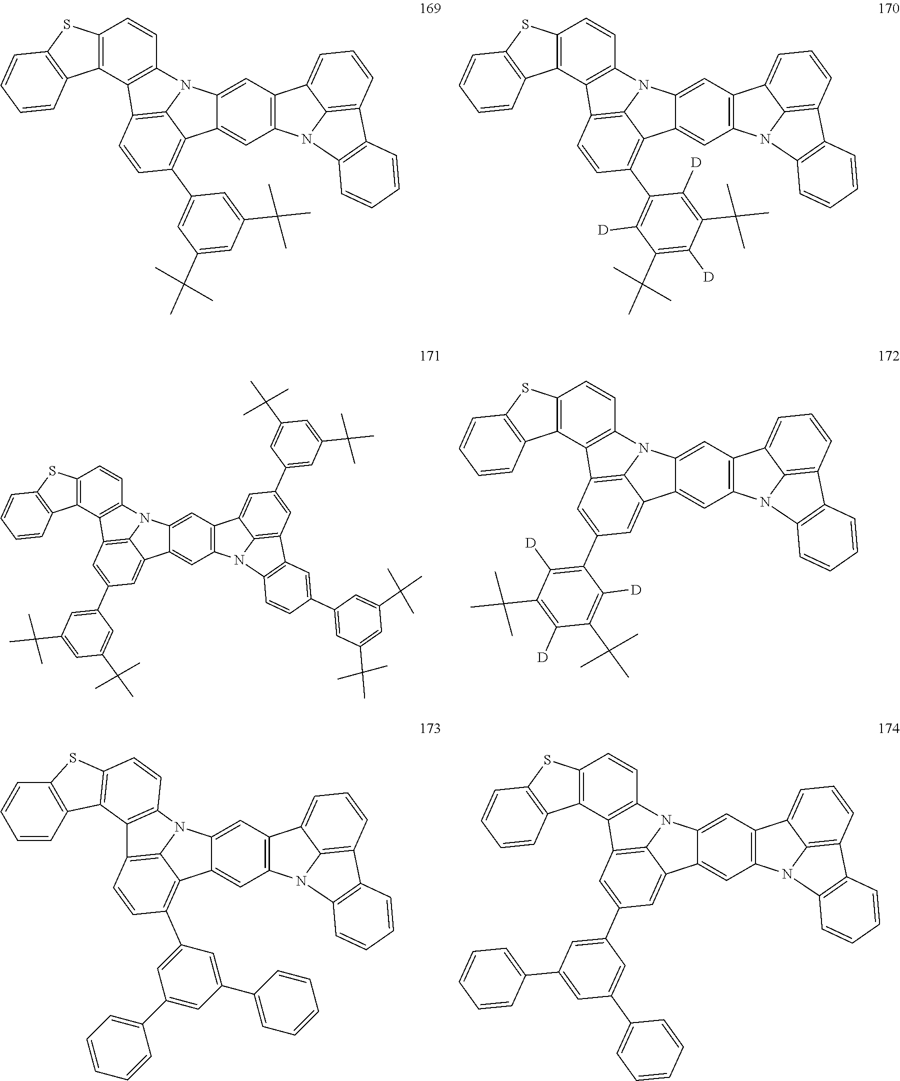 [chemical structure]
